(19) United States
(12) Patent Application Publication
(10) Pub. No.: US 2025/0223306 A1
(43) Pub. Date: Jul. 10, 2025

(54) TEAD INHIBITORS AND METHODS OF USE

(71) Applicant: Sporos BioDiscovery, Inc., Houston, TX (US)

(72) Inventors: Florian Muller, Houston, TX (US); Dora Toledo Warshaviak, Agoura Hills, CA (US); Andrew Morley, Cheshire (GB); Sharon Shacham, Sunny Isle, FL (US)

(21) Appl. No.: 18/849,268

(22) PCT Filed: Mar. 22, 2023
(86) PCT No.: PCT/US2023/015981

§ 371 Date: Sep. 20, 2024

Related U.S. Application Data
(60) Provisional application No. 63/322,600, filed on Mar. 22, 2022.

(51) Int. Cl. C07F 9/60; A61K 31/166; A61K 31/4402; A61K 31/4709; A61K 31/497; A61K 31/501; A61K 31/506; A61K 31/675; A61P 35/00; C07C 233/65; C07D 213/40; C07D 215/36; C07D 215/54; C07D 401/12; C07D 413/12

(52) U.S. Cl. CPC C07F 9/60; A61K 31/166; A61K 31/4402; A61K 31/4709; A61K 31/497; A61K 31/501; A61K 31/506; A61K 31/675; A61P 35/00; C07C 233/65; C07D 213/40; C07D 215/36; C07D 215/54; C07D 401/12; C07D 413/12

(57) ABSTRACT

The present disclosure provides, in part, compounds of formula (I), wherein the variables are as defined herein, pharmaceutical compositions comprising the compounds, and methods of using the compounds to treat physiological disorders, such as proliferative disorders, mediated by TEA domain transcription factors.

### CROSS REFERENCE TO RELATED APPLICATIONS

This application is the National Stage entry of International Application No. PCT/US2023/015981, filed Mar. 22, 2023, which claims priority to and the benefit of U.S. Provisional Application No. 63/322,600, filed Mar. 22, 2022, the contents of each of which are hereby incorporated by reference in their entirety.

### SUMMARY OF THE DISCLOSURE

Disclosed herein, in certain embodiments, are compounds of formula (I) or formula (I′), e.g., compounds of formula (Ia), formula (Ia′), formula (Ib), formula (Ic), formula (Ib-1), or formula (Ib-2), or pharmaceutically acceptable salts thereof. Additionally, disclosed herein, in certain embodiments, are compositions comprising a compound of formula (I) or formula (I′), e.g., a compound of formula (Ia), formula (Ia′), formula (Ib), formula (Ic), formula (Ib-1), or formula (Ib-2) or a pharmaceutically acceptable salt thereof, and a pharmaceutically acceptable excipient. Further disclosed herein, in certain embodiments, are methods of inhibiting abnormal transcriptional enhanced associate (TEA) domain transcription factor (TEAD) transcriptional complex activity and thus treating certain diseases or disorders such as cancer characterized by abnormal TEAD transcriptional complex activity (e.g., hyperactivation).

Disclosed herein, in certain embodiments, are compounds of formula (I):

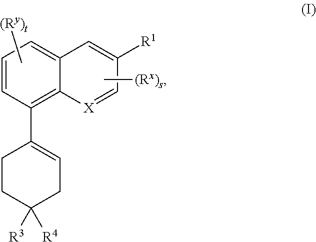

or pharmaceutically acceptable salts thereof, wherein the variables are as defined herein.

Also, disclosed herein, in certain embodiments, are compounds of formula (I′):

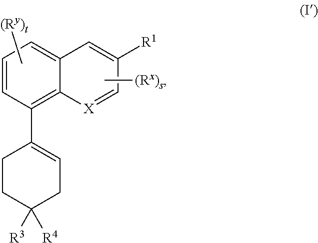

or pharmaceutically acceptable salts thereof, wherein the variables are as defined herein.

Also, disclosed herein, in certain embodiments, are compounds of formula (Ia):

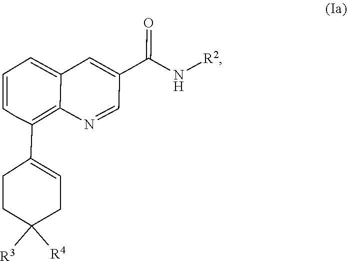

or pharmaceutically acceptable salts thereof, wherein the variables are as defined herein.

Also, disclosed herein, in certain embodiments, are compounds of formula (Ia′):

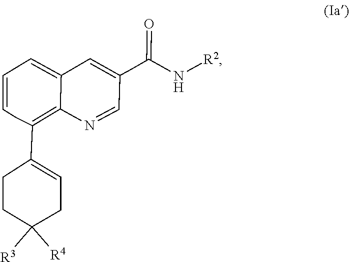

or pharmaceutically acceptable salts thereof, wherein the variables are as defined herein.

In some embodiments, the compounds of formula (I), formula (I′), formula (Ia), formula (Ia′), formula (Ib), formula (Ic), formula (Ib-1), or formula (Ib-2) are selected from Table 1 or a pharmaceutically acceptable salt thereof.

Also provided herein, in certain embodiments, are pharmaceutical compositions comprising a compound disclosed herein or a pharmaceutically acceptable salt thereof, and a pharmaceutically acceptable excipient.

Further disclosed herein, in certain embodiments, are methods of treating a disease or condition mediated by abnormal TEAD activity (e.g., disregulation) in a subject in need thereof, comprising administering to the subject a therapeutically effective amount of a compound or pharmaceutically acceptable salt described herein or a pharmaceutical composition disclosed herein. In some embodiments, the disease or condition is a cancer characterized by abnormal TEAD transcriptional complex activity (e.g., hyperactivation).

### BRIEF DESCRIPTION OF THE DRAWINGS

The disclosure can be more completely understood with reference to the following drawings.

FIG. 1 is a diagram showing the domain architecture of human TEAD1, TEAD2, TEAD3, and TEAD4. The percent values represent the identity of the N-terminal DNA binding domain (DNA-BD) and C-terminal YAP/TAZ binding domain (YAP/TAZ-BD) of TEAD2-4 compared to the respective binding domains of TEAD1. Post translation modifications including palmitoylation and phosphorylation as well as the p38 binding D domain of DNA-BD are also shown.

FIG. 2A is a diagram showing the upstream signaling and downstream transcriptional outputs of TEAD in cancer biology, which regulate critical functions in tumorigenesis, stem cell maintenance, cancer immunology, and metabolism, as well as formation of signaling feedback loops. Oncogenic signal transduction pathways include EGFR signaling, TGFβ signaling, WNt signaling, GPCR signaling, and cancer genes (indicated with *), such as KRAS, BRAF, LKB1, APC, and GNAQ/11.

FIG. 2B is a diagram showing the role of TEAD in multiple stages of tumorigenesis.

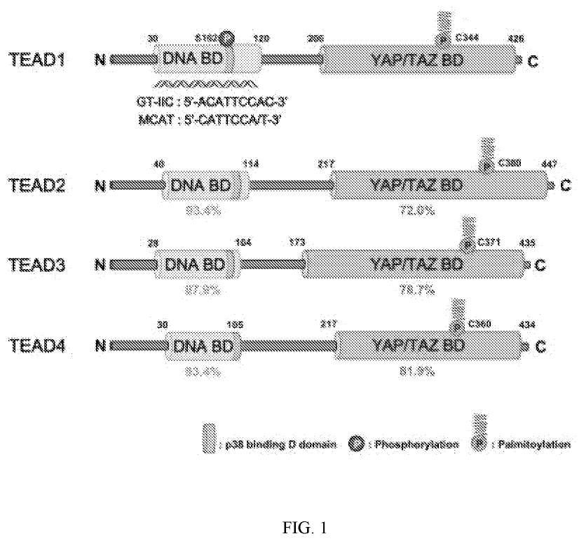

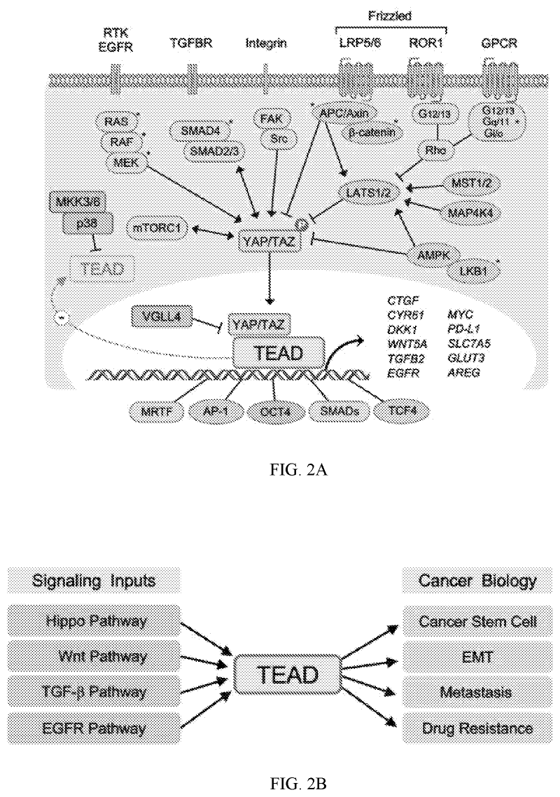

### DETAILED DESCRIPTION

Provided herein, in certain embodiments, are compounds (e.g., compounds of formula (I) or formula (I′), e.g., compounds of formula (Ia), formula (Ia′), formula (Ib), formula (Ic), formula (Ib-1), or formula (Ib-2), or pharmaceutically acceptable salts thereof. Further provided herein, in certain embodiments are compositions comprising a compound of formula (I) or formula (I′), e.g., a compound of formula (Ia), formula (Ia′), formula (Ib), formula (Ic), formula (Ib-1), or formula (Ib-2), or a pharmaceutically acceptable salt thereof, and a pharmaceutically acceptable excipient). Also provided herein, in certain embodiments, are methods of use of compounds and compositions disclosed herein. The contemplated compounds and compositions disclosed herein are inhibitors of transcriptional enhancer factor domain (TEAD) activity and thus are useful in methods of treating certain diseases or disorders such as cancer characterized by abnormal TEAD transcriptional complex activity (e.g., hyperactivation).

The present disclosure provides compounds (e.g., compounds of formula (I) or formula (I′), e.g., compounds of formula (Ia), formula (Ia′), formula (Ib), formula (Ic), formula (Ib-1), or formula (Ib-2)) that can selectively bind certain TEAD isoforms associated with therapeutic efficacy (e.g., TEAD1 and/or TEAD4). Such selective binding is advantageous because less selective and/or non-selective TEAD isoform binding may result in an increase in undesirable effects (e.g., toxicity and/or cancer cell proliferation).

### Certain Terminology

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligo- or polynucleotide chemistry and hybridization described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

### Chemical Definitions

Definitions of specific functional groups and chemical terms are described in more detail below.

In some embodiments, compounds described herein comprise one or more asymmetric centers, and thus exist in various isomeric forms, e.g., enantiomers and/or diastereomers. For example, in some embodiments, the compounds described herein are in the form of an individual enantiomer, diastereomer or geometric isomer, or are in the form of a mixture of stereoisomers, including racemic mixtures and mixtures enriched in one or more stereoisomer. In some embodiments, isomers are isolated from mixtures by methods known to those skilled in the art, including chiral high pressure liquid chromatography (HPLC) and the formation and crystallization of chiral salts; or preferred isomers are prepared by asymmetric syntheses. The disclosure additionally encompasses compounds described herein as individual isomers substantially free of other isomers, and alternatively, as mixtures of various isomers.

In some embodiments, compound described herein also comprise one or more isotopic substitutions. For example, in some embodiments, H is in any isotopic form, including ¹H, ²H (D or deuterium), and ³H (T or tritium); C is any isotopic form, including ¹²C, ¹³C, and ¹⁴C; O is in any isotopic form, including ¹⁶O and ¹⁸O; F is in any isotopic form, including ¹⁸F and ¹⁹F; and the like.

The articles “a” and “an” are used herein to refer to one or to more than one (i.e. at least one) of the grammatical objects of the article.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

As used herein, “alkylene,” refers to a divalent radical of an alkyl group. When a range or number of carbons is provided for a particular “alkylene” group, it is understood that the range or number refers to the range or number of carbons in the linear carbon divalent chain. In some embodiments, “alkylene” group is substituted or unsubstituted with one or more substituents as described herein.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”).

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In some embodiments, in heteroaryl groups that contain one or more nitrogen atoms, the point of attachment is a carbon or nitrogen atom, as valency permits.

In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively.

The term “cycloalkyl” refers to a monovalent saturated cyclic, bicyclic, or bridged cyclic (e.g., adamantyl) hydrocarbon group of 3-12, 3-10, 3-8, 4-8, or 4-6 carbons, referred to herein, e.g., as “C₃-C₁₀cycloalkyl,” derived from a cycloalkane. Exemplary cycloalkyl groups include, but are not limited to, cyclohexanes, cyclopentanes, cyclobutanes and cyclopropanes.

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, and sulfur (“3-10 membered heterocyclyl”). In some embodiments, in heterocyclyl groups that contain one or more nitrogen atoms, the point of attachment is a carbon or nitrogen atom, as valency permits. In some embodiments, a heterocyclyl group is either be monocyclic (“monocyclic heterocyclyl”) or a fused, bridged or spiro ring system such as a bicyclic system (“bicyclic heterocyclyl”), and is saturated or is partially unsaturated. In some embodiments, heterocyclyl bicyclic ring systems include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more phenyl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” are used interchangeably.

The term “carbocyclyl” or “carbocyclic” refers to a radical of a non-aromatic cyclic hydrocarbon group having from 3 to 10 ring carbon atoms (“3-10 membered carbocyclic ring”) and zero heteroatoms in the non-aromatic ring system. In some embodiments, “carbocyclyl” or “carbocyclic” ring is a “3-7 membered carbocyclic ring.”

In some embodiments, a heterocyclyl group is a 3-10 membered non-aromatic ring system having ring carbon atoms and 1-4 ring heteroatoms, wherein each heteroatom is independently selected from nitrogen, oxygen, and sulfur (“3-10 membered heterocyclyl”). In some embodiments, a heterocyclyl group is a 4-7 membered non-aromatic ring system having ring carbon atoms and 1-4 ring heteroatoms, wherein each heteroatom is independently selected from nitrogen, oxygen, and sulfur (“4-7 membered heterocyclyl”). In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

“Hetero” when used to describe a compound or a group present on a compound means that one or more carbon atoms in the compound or group have been replaced by a nitrogen, oxygen, or sulfur heteroatom. In some embodiments, hetero is applied to any of the hydrocarbyl groups described above such as alkyl, e.g., heteroalkyl; cycloalkyl, e.g., heterocyclyl; aryl, e.g., heteroaryl; and the like having from 1 to 5, and particularly from 1 to 3 heteroatoms.

The terms “halo” and “halogen” as used herein refer to an atom selected from fluorine (fluoro, —F), chlorine (chloro, —Cl), bromine (bromo, —Br), and iodine (iodo, —I). In certain embodiments, the halo group is either fluoro or chloro.

The term “haloalkyl” includes mono, poly, and perhaloalkyl groups substituted with one or more halogen atoms where the halogens are independently selected from fluorine, chlorine, bromine, and iodine.

The term “oxo” refers to ═O.

In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

Nitrogen atoms are substituted or unsubstituted as valency permits, and include primary, secondary, tertiary, and quaternary nitrogen atoms.

These and other exemplary substituents are described in more detail in the Detailed Description, Examples, and Claims. The disclosure is not intended to be limited in any manner by the above exemplary listing of substituents.

### Other Definitions

As used herein, “pharmaceutically acceptable excipient” refers to any substance in a pharmaceutical formulation other than the active pharmaceutical ingredient(s). Exemplary pharmaceutical excipients include those that aid the manufacturing process; protect, support or enhance stability; increase bioavailability; or increase patient acceptability. They may also assist in product identification or enhance the overall safety or function of the product during storage or use.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” “individual” and “subject” are used interchangeably herein. None of the terms require the supervision of medical personnel.

The terms “disease”, “disorder”, and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. In some embodiments, the term “therapeutically effective amount” encompasses an amount that improves overall therapy, reduces or avoids symptoms or causes of disease or condition, or enhances the therapeutic efficacy of another therapeutic agent.

### Compounds

Disclosed herein, in certain embodiments, are compounds of formula (I):

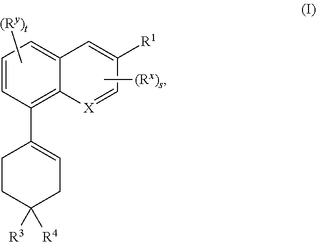

or a pharmaceutically acceptable salt thereof, wherein:
X is N or CH;
R¹ is is selected from the group consisting of —C(O)OR⁵, —C(O)—NR⁶R², —S(O)₂—N(R⁶)₂, —S(O)ₘ—(C₁₋₆alkyl), and —S(O)N(R⁶)₂;
R² is selected from the group consisting of —C₁-C₆ alkyl, —(C₁-C₆ alkyl substituted with one or two —OR⁵), —(C₁-C₆ alkylene)-CN, —(C₁-C₆ alkylene)-S(O)ₙ—(C₁-C₆ alkyl), —(C₁-C₄ alkylene optionally substituted with OR⁵)—C(O)OR⁵, —(C₁-C₄ alkylene optionally substituted with OR⁵)—C(O)N(R⁶)₂, —(C₀-C₄ alkylene)-phenyl, —(C₁-C₆ alkylene)-N(R⁶)₂, 5-6 membered heteroaryl having one, two, or three heteroatoms each independently selected from N, O, and S, —(C₁-C₄ alkylene optionally substituted with OR⁵)-(5-6 membered heteroaryl having one, two, or three heteroatoms each independently selected from N, O, and S), —(C₀-C₄ alkylene)-C₃-C₁₀cycloalkyl, and —(C₀-C₄ alkylene)-(3-10 membered heterocyclyl having one, two, three, or four heteroatoms each independently selected from N, O, and S), wherein any aforementioned phenyl, 5-6 membered heteroaryl, C₃-C₁₀cycloalkyl, and 3-10 membered heterocyclyl are optionally substituted;
m is 1 or 2;
n is 0, 1, or 2;
R³ is selected from the group consisting of hydrogen, halogen, —C₁-C₆ alkyl, —(C₁-C₆ haloalkyl), —O—(C₁-C₆ alkyl), and —O—(C₁-C₆ haloalkyl);
R⁴ is selected from the group consisting of hydrogen, halogen, and —C₁-C₆ alkyl; or
R³ and R⁴ are taken together to form a 3-7 membered carbocyclic ring with the carbon to which R³ and R⁴ are attached, wherein the carbocyclic ring is optionally substituted with one or more halogens;
each R⁵ is independently hydrogen or —C₁-C₆ alkyl;
each R⁶ is independently hydrogen or —C₁-C₆ alkyl;
each R^{x} is independently selected from the group consisting of —C₁-C₆ alkyl, halogen, —OR⁵, —CN, and —N(R⁶)₂;
each R^{y} is independently selected from the group consisting of —C₁-C₆ alkyl, halogen, —OR⁵, —CN, and —N(R⁶)₂;
s is 0, 1, or 2; and
t is 0, 1, 2, or 3.

Disclosed herein, in certain embodiments, are compounds of formula (I′):

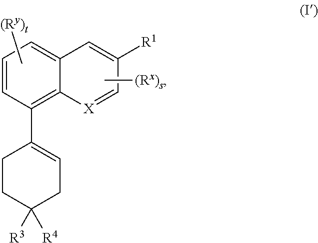

or a pharmaceutically acceptable salt thereof, wherein:
X is N or CH;
R¹ is selected from the group consisting of —C(O)OR⁵, —C(O)—NR⁶R², —S(O)₂—N(R⁶)₂, —S(O)ₘ—(C₁₋₆alkyl), and —S(O)N(R⁶)₂;
R² is selected from the group consisting of —C₁-C₆ alkyl, —(C₁-C₆ alkyl substituted with one or two —OR⁵), —(C₁-C₆ alkylene)-CN, —(C₁-C₆ alkylene)-S(O)ₙ—(C₁-C₆ alkyl), —(C₁-C₄ alkylene optionally substituted with OR⁵)—C(O)OR⁵, —(C₁-C₄ alkylene optionally substituted with CN or OR⁵)—C(O)N(R⁶)₂, —(C₀-C₄ alkylene)-phenyl, —(C₁-C₆ alkylene)-N(R⁶R⁷), —(C₁-C₆ alkylene)-OP(O)(OR⁵)₂. 5-6 membered heteroaryl having one, two, or three heteroatoms each independently selected from N, O, and S, —(C₁-C₄ alkylene optionally substituted with OR⁵)-(5-6 membered heteroaryl having one, two, or three heteroatoms each independently selected from N, O, and S), —(C₀-C₄ alkylene)-C₃-C₁₀cycloalkyl, and —(C₀-C₄ alkylene)-(3-10 membered heterocyclyl having one, two, three, or four heteroatoms each independently selected from N, O, and S), wherein any aforementioned phenyl, 5-6 membered heteroaryl, C₃-C₁₀cycloalkyl, and 3-10 membered heterocyclyl are optionally substituted with one or more R″;
m is 1 or 2;
n is 0, 1, or 2;
R³ is selected from the group consisting of hydrogen, halogen, —C₁-C₆ alkyl, —(C₁-C₆ haloalkyl), —O—(C₁-C₆ alkyl), and —O—(C₁-C₆ haloalkyl);
R⁴ is selected from the group consisting of hydrogen, halogen, and —C₁-C₆ alkyl; or
R³ and R⁴ are taken together to form a 3-7 membered carbocyclic ring with the carbon to which R³ and R⁴ are attached, wherein the carbocyclic ring is optionally substituted with one or more halogens;
each R⁵ is independently hydrogen or —C₁-C₆ alkyl;
each R⁶ is independently hydrogen or —C₁-C₆ alkyl;
R⁷ is selected from the group consisting of hydrogen, —C₁-C₆ alkyl, —C(O)—(C₁₋₆ alkyl), —C(O)N(R⁶)₂, —C(O)₂—(C₁₋₆ alkyl), —S(O)ₙ—(C₁-C₆ alkyl), and —S(O)ₙNR⁶—(C₁-C₆ alkyl);
each R″ is independently selected from the group consisting of —C₁-C₆ alkyl, halogen, —N(R⁶)₂, and oxo, wherein the —C₁-C₆ alkyl is optionally substituted with —OH;
each R^{x} is independently selected from the group consisting of —C₁-C₆ alkyl, halogen, —OR⁵, and —CN;
each R^{y} is independently selected from the group consisting of —C₁-C₆ alkyl, halogen, —OR⁵, and —CN;
s is 0, 1, or 2; and
t is 0, 1, 2, or 3.

In some embodiments, X is N. In some embodiments, X is CH.

In some embodiments, R¹ is —C(O)—NHR². In some embodiments, R¹ is —S(O)—R². In some embodiments, R¹ is —C(O)OH. In some embodiments, R¹ is —S(O)₂—R². In some embodiments, R¹ is —S(O)₂—NHR². In some embodiments, R¹ is —S(O)CH₃. In some embodiments, R¹ is —S(O)₂CH₃. In some embodiments, R¹ is —S(O)₂NHCH₃.

In some embodiments, R² is selected from the group consisting of —C₁-C₆ alkyl, —(C₁-C₆ alkyl substituted with one or two —OR⁵), —(C₁-C₆ alkylene)-CN, —(C₁-C₆ alkylene)-S(O)ₙ—(C₁-C₆ alkyl), —(C₁-C₄ alkylene optionally substituted with OR⁵)—C(O)OR⁵, —(C₁-C₄ alkylene optionally substituted with OR⁵)—C(O)N(R⁶)₂, —(C₁-C₆ alkylene)-N(R⁶)₂, and —(C₁-C₄ alkylene optionally substituted with OR⁵)-(5-6 membered heteroaryl having one, two, or three heteroatoms each independently selected from N, O, and S).

In some embodiments, R² is —(C₁-C₄ alkylene)-5-6 membered heteroaryl.

In some embodiments, R² is —CH(CH₃)-5-6 membered heteroaryl.

In some embodiments, R² is —CH₂-5-6 membered heteroaryl.

In some embodiments, the 5-6 membered heteroaryl is optionally substituted with C₁-C₆alkyl or N(R³)₂, wherein each R^{a} is independently hydrogen or C₁-C₆alkyl.

In some embodiments, the 5-6 membered heteroaryl is pyridyl. In some embodiments, the the 5-6 membered heteroaryl is

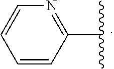 [SMILES: *c1ccccn1]

In some embodiments, the 5-6 membered heteroaryl is oxazolyl. In some embodiments, the 5-6 membered heteroaryl is

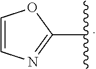 [SMILES: *c1ncco1]

In some embodiments, R² is C₁-C₆alkyl. In some embodiments, R² is —(C₁-C₆ alkyl)-OH. In some embodiments, R² is —(C₁-C₆ alkyl)-O—(C₁-C₆ alkyl). In some embodiments, R² is isopropyl. In some embodiments, R² is —(C₁-C₆ alkyl substituted with one or two —OR⁵). In some embodiments, R² is —(C₁-C₆ alkyl substituted with —OR⁵). In some embodiments, R² is —(C₁-C₆ alkyl substituted with —OH). In some embodiments, R² is —(C₁-C₆ alkyl substituted with —OH and —OCH₃). In some embodiments, R² is —(C₁-C₆ alkylene)-S(O)ₙ—(C₁-C₆ alkyl). In some embodiments, R² is —(C₁-C₆ alkylene)-CN. In some embodiments, R² is —(C₁-C₄ alkylene optionally substituted with OR⁵)—C(O)OR⁵. In some embodiments, R² is —(C₁-C₄ alkylene optionally substituted with OR⁵)—C(O)N(R⁶)₂. In some embodiments, R² is —(C₁-C₆ alkylene)-N(R⁶)₂.

In some embodiments, R² is selected from the group consisting of methyl,

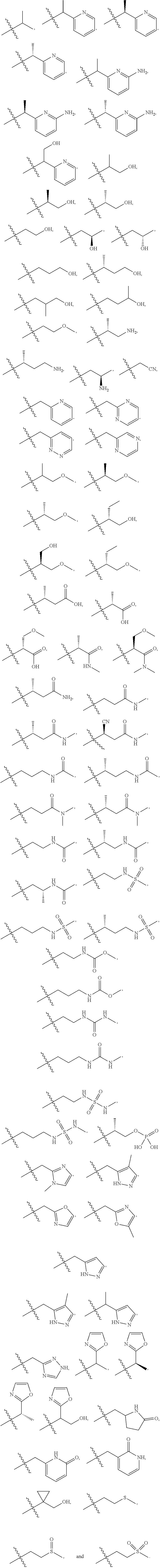

In some embodiments, R² is selected from the group consisting of methyl,

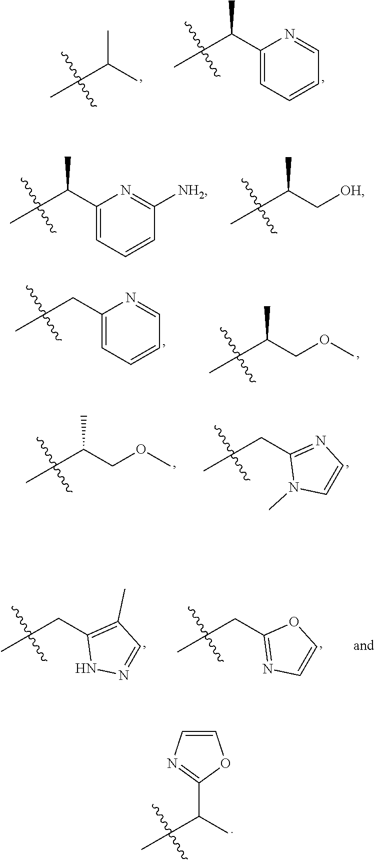

In some embodiments, R² is selected from the group consisting of methyl,

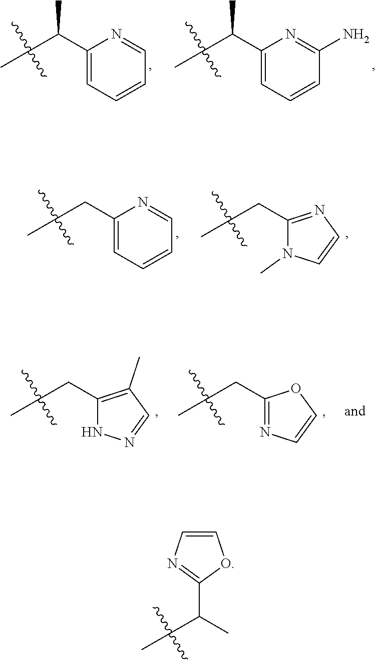

In some embodiments, m is 1. In some embodiments, m is 2.

In some embodiments, n is 0. In some embodiments, n is 1. In some embodiments, n is 2.

In some embodiments, s is 0. In some embodiments, s is 1. In some embodiments, s is 2.

In some embodiments, t is 0. In some embodiments, t is 1. In some embodiments, t is 2. In some embodiments, t is 3.

In some embodiments, R³ is hydrogen. In some embodiments, R³ is —C₁-C₆ alkyl. In some embodiments, R³ is methyl.

In some embodiments, R³ is —C₁-C₆ haloalkyl.

In some embodiments, R³ is trifluoromethyl.

In some embodiments, wherein R³ is halogen. In some embodiments, R³ is —F.

In some embodiments, R⁴ is hydrogen. In some embodiments, R⁴ is —C₁-C₆ alkyl. In some embodiments, R⁴ is methyl. In some embodiments, R⁴ is halogen. In some embodiments, R⁴ is —F.

In some embodiments, R³ is —C₁-C₆ haloalkyl and R⁴ is H. In some embodiments, R³ and R⁴ are both halogen. In some embodiments, R³ and R⁴ are both-C₁-C₆ alkyl. In some embodiments, R³ and R⁴ are both H.

In some embodiments, R³ and R⁴ are taken together to form a 3-7 membered carbocyclic ring with the carbon to which R³ and R⁴ are attached, wherein the carbocyclic ring is optionally substituted with one or more halogens. In some embodiments, the halogen is F.

In some embodiments, R³ and R⁴ are taken together to form cyclopropyl or cyclobutyl optionally substituted with one or more halogens. In some embodiments, R³ and R⁴ are taken together to form cyclopropyl or cyclobutyl optionally substituted with one or more fluoro. In some embodiments, R³ and R⁴ are taken together to form cyclopropyl. In some embodiments, R³ and R⁴ are taken together to form cyclobutyl optionally substituted with one or more fluoro.

In some embodiments, R⁵ is hydrogen. In some embodiments, R⁵ is —C₁-C₆ alkyl. In some embodiments, R⁵ is methyl.

In some embodiments, R⁶ is hydrogen. In some embodiments, R⁶ is —C₁-C₆ alkyl.

In some embodiments, s and t are both 0.

Disclosed herein, in certain embodiments, are compounds of formula (Ia):

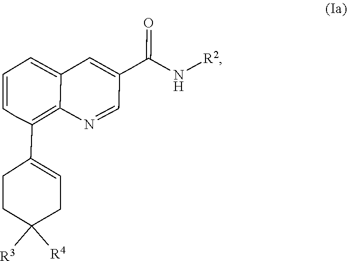

or a pharmaceutically acceptable salt thereof, wherein:
R² is selected from the group consisting of —C₁-C₆ alkyl, —(C₁-C₆ alkyl substituted with one or two —OR⁵), —(C₁-C₆ alkylene)-CN, —(C₁-C₆ alkylene)-S(O)ₙ—(C₁-C₆ alkyl), —(C₁-C₄ alkylene optionally substituted with CN or OR⁵)—C(O)OR⁵, —(C₁-C₄ alkylene optionally substituted with OR⁵)—C(O)N(R⁶)₂, —(C₁-C₆ alkylene)-N(R⁶R⁷), and —(C₁-C₄ alkylene optionally substituted with OR⁵)-(5-6 membered heteroaryl having one, two, or three heteroatoms each independently selected from N, O, and S), wherein the 5-6 membered heteroaryl is optionally substituted with one or more R^{w};
n is 0, 1, or 2;
R³ is selected from the group consisting of halogen, —C₁-C₆ alkyl, —C₁-C₆ haloalkyl, and —O—(C₁-C₆ alkyl);
R⁴ is selected from the group consisting of hydrogen, halogen, and —C₁-C₆ alkyl; or
R³ and R⁴ are taken together to form a 3-7 membered carbocyclic ring with the carbon to which R³ and R⁴ are attached, wherein the 3-7 membered carbocyclic ring is optionally substituted with one or more halogens;
each R⁵ is independently for each occurrence, hydrogen or —C₁-C₆ alkyl; and
each R⁶ is independently for each occurrence, hydrogen or —C₁-C₆ alkyl
R⁷ is selected from the group consisting of hydrogen, —C₁-C₆ alkyl, —C(O)—(C₁₋₆ alkyl), —C(O)NR⁶, —C(O)₂—(C₁₋₆ alkyl), and —S(O)₂NR⁶—(C₁-C₆ alkyl);
each R^{w} is independently selected from the group consisting of —C₁-C₆ alkyl, —N(R⁶)₂, and oxo, wherein the —C₁-C₆ alkyl is optionally substituted with —OH.

Disclosed herein, in certain embodiments, are compounds of formula (Ia′):

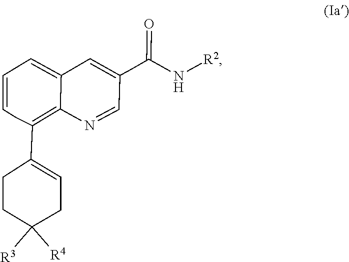

or a pharmaceutically acceptable salt thereof, wherein:
R² is selected from the group consisting of —C₁-C₆ alkyl, —(C₁-C₆ alkyl substituted with one or two —OR⁵), —(C₁-C₆ alkylene)-CN, —(C₁-C₆ alkylene)-S(O)ₙ—(C₁-C₆ alkyl), —(C₁-C₄ alkylene optionally substituted with CN or OR⁵)—C(O)OR⁵, —(C₁-C₄ alkylene optionally substituted with OR⁵)—C(O)N(R⁶)₂, —(C₁-C₆ alkylene)-N(R⁶R⁷), —(C₁-C₆ alkylene)-OP(O)(OR⁵)₂, and —(C₁-C₄ alkylene optionally substituted with OR⁵)-(5-6 membered heteroaryl having one, two, or three heteroatoms each independently selected from N, O, and S), wherein the 5-6 membered heteroaryl is optionally substituted with one or more R^{w};
n is 0, 1, or 2;
R³ is selected from the group consisting of halogen, —C₁-C₆ alkyl, —C₁-C₆ haloalkyl, and —O—(C₁-C₆ alkyl);
R⁴ is selected from the group consisting of hydrogen, halogen, and —C₁-C₆ alkyl; or
R³ and R⁴ are taken together to form a 3-7 membered carbocyclic ring with the carbon to which R³ and R⁴ are attached, wherein the 3-7 membered carbocyclic ring is optionally substituted with one or more halogens;
each R⁵ is independently for each occurrence, hydrogen or —C₁-C₆ alkyl; and
each R⁶ is independently for each occurrence, hydrogen or —C₁-C₆ alkyl
R⁷ is selected from the group consisting of hydrogen, —C₁-C₆ alkyl, —C(O)—(C₁₋₆ alkyl), —C(O)N(R⁶)₂, —C(O)₂—(C₁₋₆ alkyl), —S(O)ₙ—(C₁-C₆ alkyl), and —S(O)ₙNR⁶—(C₁-C₆ alkyl);
each R^{w} is independently selected from the group consisting of —C₁-C₆ alkyl, —N(R⁶)₂, and oxo, wherein the —C₁-C₆ alkyl is optionally substituted with —OH.

In some embodiments, the compound is a compound of formula (Ib):

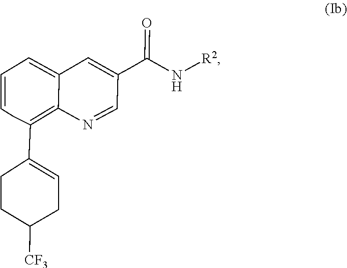

or a pharmaceutically acceptable salt thereof.

In some embodiments, the compound is a compound of formula (Ic):

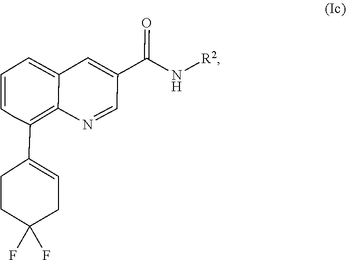

or a pharmaceutically acceptable salt thereof.

In some embodiments, the compound is a compound of formula (Ib-1):

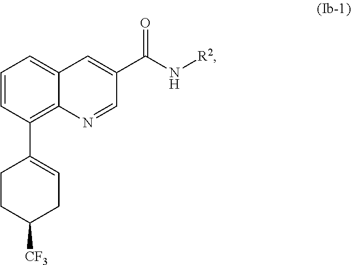

or a pharmaceutically acceptable salt thereof.

In some embodiments, the compound is a compound of formula (Ib-2):

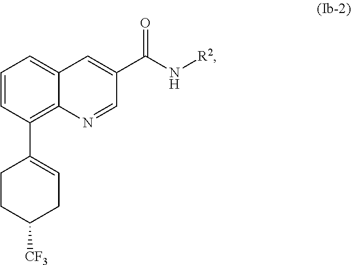

or a pharmaceutically acceptable salt thereof.

In some embodiments, R² is —(C₁-C₄ alkylene)-5-6 membered heteroaryl.

In some embodiments, R² is —CH(CH₃)-5-6 membered heteroaryl.

In some embodiments, R² is —CH₂-5-6 membered heteroaryl.

In some embodiments, the 5-6 membered heteroaryl is optionally substituted with C₁-C₆alkyl or N(R^{a})₂, wherein each R^{a} is independently hydrogen or C₁-C₆alkyl.

In some embodiments, the 5-6 membered heteroaryl is pyridyl. In some embodiments, the 5-6 membered heteroaryl is

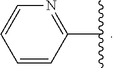 [SMILES: *c1ccccn1]

In some embodiments, the 5-6 membered heteroaryl is oxazolyl. In some embodiments, the 5-6 membered heteroaryl is

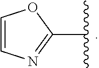 [SMILES: *c1ncco1]

In some embodiments, R² is C₁-C₆alkyl. In some embodiments, R² is —(C₁-C₆ alkyl)-OH. In some embodiments, R² is —(C₁-C₆ alkyl)-O—(C₁-C₆ alkyl). In some embodiments, R² is isopropyl. In some embodiments, R² is —(C₁-C₆ alkyl substituted with one or two —OR⁵). In some embodiments, R² is —(C₁-C₆ alkyl substituted with —OR⁵). In some embodiments, R² is —(C₁-C₆ alkyl substituted with —OH). In some embodiments, R² is —(C₁-C₆ alkyl substituted with —OH and —OCH₃). In some embodiments, R² is —(C₁-C₆ alkylene)-S(O)ₙ—(C₁-C₆ alkyl). In some embodiments, R² is —(C₁-C₆ alkylene)-CN. In some embodiments, R² is —(C₁-C₄ alkylene optionally substituted with OR⁵)—C(O)OR⁵. In some embodiments, R² is —(C₁-C₄ alkylene optionally substituted with OR⁵)—C(O)N(R⁶)₂. In some embodiments, R² is —(C₁-C₆ alkylene)-OP(O)(OR⁵)₂. In some embodiments, R² is —(C₁-C₆ alkylene)-N(R⁶)₂.

In some embodiments, R² is selected from the group consisting of —(C₁-C₆ alkyl substituted with one or two —OR⁵), —(C₁-C₆ alkylene)-S(O)ₙ—(C₁-C₆ alkyl), —(C₁-C₄ alkylene optionally substituted with OR⁵)—C(O)OR⁵, —(C₁-C₄ alkylene optionally substituted with OR⁵)—C(O)N(R⁶)₂, —(C₁-C₆ alkylene)-N(R⁶R⁷)₂, —(C₁-C₆ alkylene)-OP(O)(OR⁵)₂, —(C₁-C₄ alkylene optionally substituted with OR⁵)-(5-6 membered heteroaryl having one, two, or three heteroatoms each independently selected from N, O, and S), and —(C₀-C₄ alkylene)-(3-10 membered heterocyclyl having one, two, three, or four heteroatoms each independently selected from N, O, and S), wherein any aforementioned 5-6 membered heteroaryl and 3-10 membered heterocyclyl are optionally substituted with 1, 2, 3, or 4 substituents each independently selected from the group consisting of methyl, —NH₂, and oxo.

In some embodiments, R² is selected from the group consisting of —(C₁-C₆ alkyl substituted with one or two —OR⁵), —(C₁-C₆ alkylene)-N(R⁶R⁷)₂, and —(C₁-C₄ alkylene optionally substituted with OR⁵)-(5-6 membered heteroaryl having one, two, or three heteroatoms each independently selected from N, O, and S), wherein any aforementioned 5-6 membered heteroaryl and 3-10 membered heterocyclyl are optionally substituted with 1, 2, 3, or 4 substituents each independently selected from the group consisting of methyl, —NH₂, and oxo.

In some embodiments, R² is selected from the group consisting of methyl,

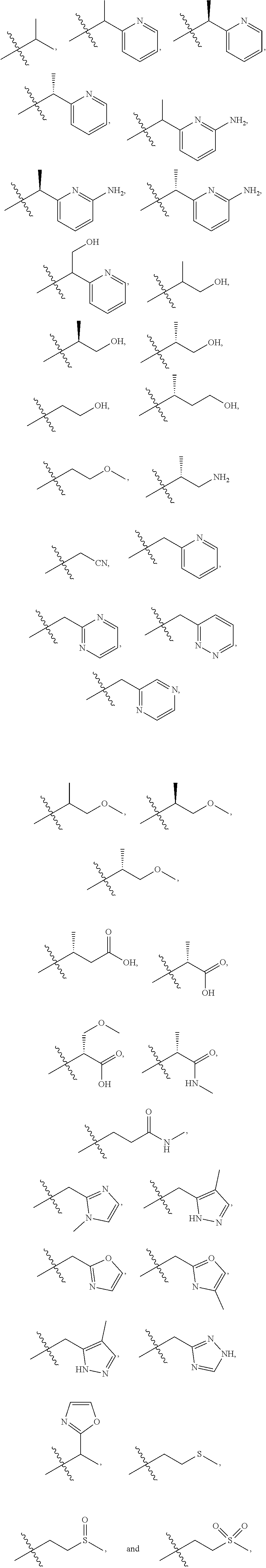

In some embodiments, R² is selected from the group consisting of methyl,

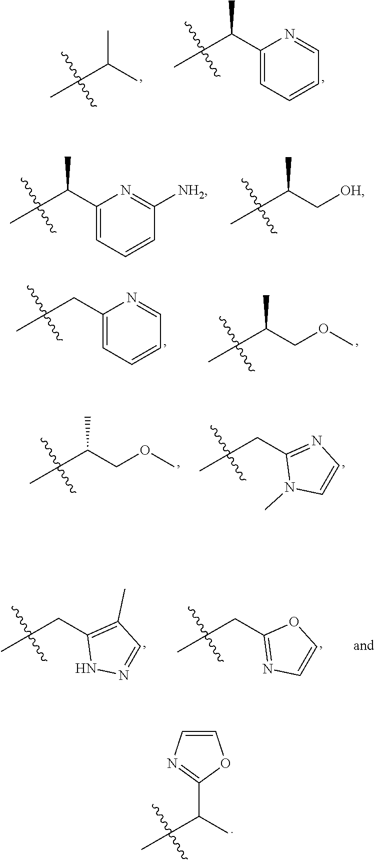

In some embodiments, R² is selected from the group consisting of methyl,

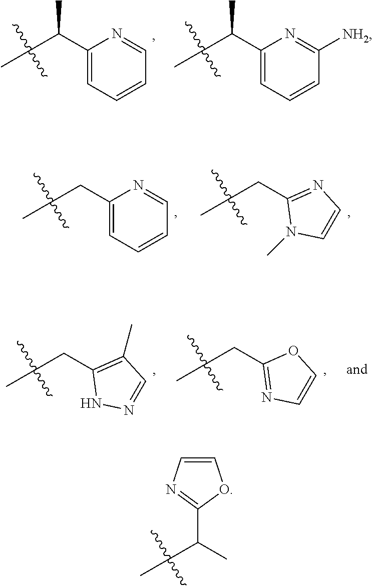

In some embodiments, R³ is —C₁-C₆ haloalkyl.

In some embodiments, R³ is trifluoromethyl.

In some embodiments, R³ is halogen. In some embodiments, R³ is —F.

In some embodiments, R⁴ is hydrogen. In some embodiments, R⁴ is halogen. In some embodiments, R⁴ is —F.

In some embodiments, R³ and R⁴ are taken together to form a 3-7 membered carbocyclic ring with the carbon to which R³ and R⁴ are attached, wherein the carbocyclic ring is optionally substituted with one or more halogens. In some embodiments, the halogen is F.

In some embodiments, the compound is selected from Table 1:

TABLE 1
List of compounds.

| Compound | |
|---|---|
| No. | Structure |
| 1 | 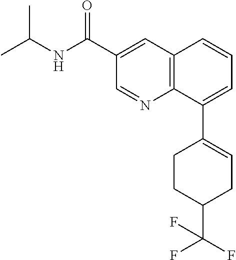 [SMILES: CC(C)NC(=O)c1cnc2c(C3=CCC(C(F)(F)F)CC3)cccc2c1] |
| 2 | 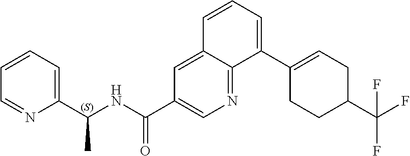 |
| 3 | 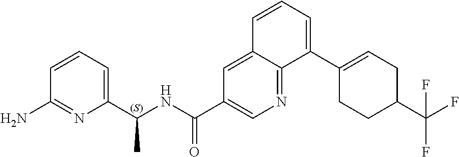 |
| 4 | 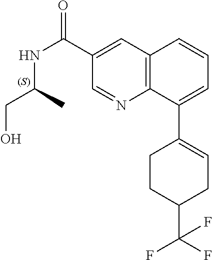 |
| 5 | 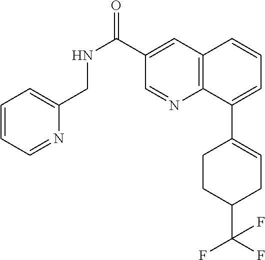 [SMILES: O=C(NCc1ccccn1)c1cnc2c(C3=CCC(C(F)(F)F)CC3)cccc2c1] |
| 6 | 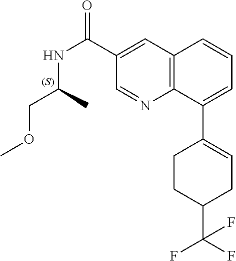 |
| 7 | 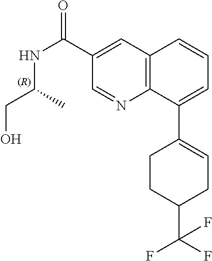 |
| 8 | 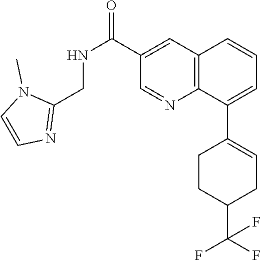 [SMILES: Cn1ccnc1CNC(=O)c1cnc2c(C3=CCC(C(F)(F)F)CC3)cccc2c1] |
| 9 | 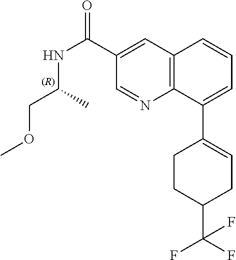 |
| 10 | 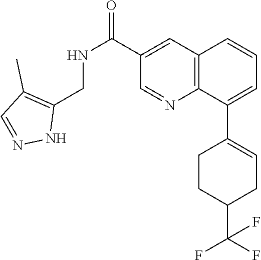 [SMILES: Cc1cn[nH]c1CNC(=O)c1cnc2c(C3=CCC(C(F)(F)F)CC3)cccc2c1] |
| 11 | 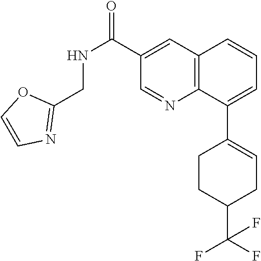 [SMILES: O=C(NCc1ncco1)c1cnc2c(C3=CCC(C(F)(F)F)CC3)cccc2c1] |
| 12 | 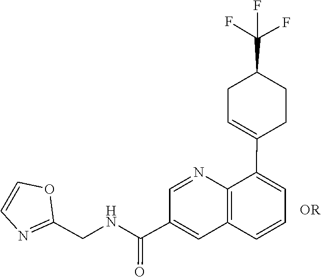 [SMILES: O=C(NCc1ncco1)c1cnc2c(C3=CC[C@@H](C(F)(F)F)CC3)cccc2c1] |
| | 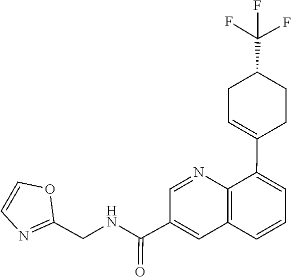 [SMILES: O=C(NCc1ncco1)c1cnc2c(C3=CC[C@H](C(F)(F)F)CC3)cccc2c1] |
| 13 | 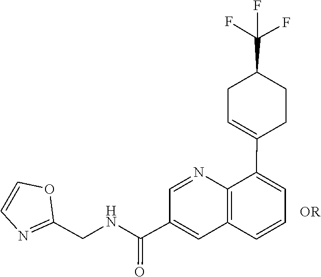 [SMILES: O=C(NCc1ncco1)c1cnc2c(C3=CC[C@@H](C(F)(F)F)CC3)cccc2c1] |
| | 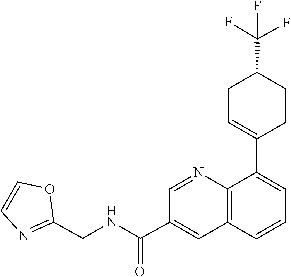 [SMILES: O=C(NCc1ncco1)c1cnc2c(C3=CC[C@H](C(F)(F)F)CC3)cccc2c1] |
| 14 | 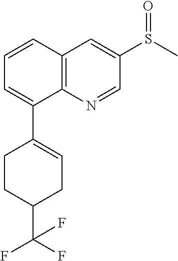 [SMILES: CS(=O)c1cnc2c(C3=CCC(C(F)(F)F)CC3)cccc2c1] |
| 15 | 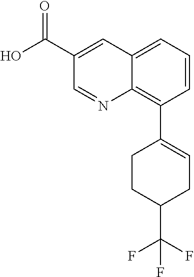 [SMILES: O=C(O)c1cnc2c(C3=CCC(C(F)(F)F)CC3)cccc2c1] |
| 16 | 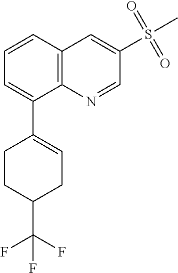 [SMILES: CS(=O)(=O)c1cnc2c(C3=CCC(C(F)(F)F)CC3)cccc2c1] |
| 17 | 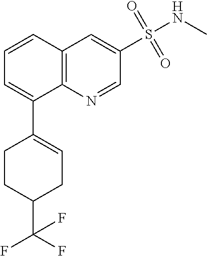 [SMILES: CNS(=O)(=O)c1cnc2c(C3=CCC(C(F)(F)F)CC3)cccc2c1] |
| 18 | 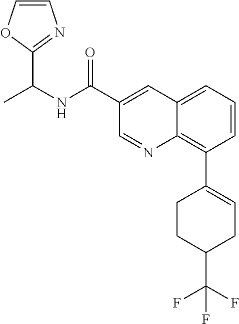 [SMILES: CC(NC(=O)c1cnc2c(C3=CCC(C(F)(F)F)CC3)cccc2c1)c1ncco1] |
| 19 | 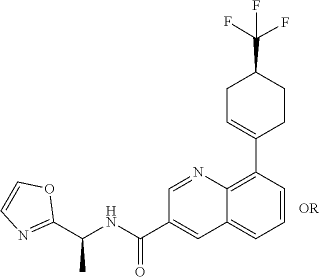 [SMILES: C[C@H](NC(=O)c1cnc2c(C3=CC[C@@H](C(F)(F)F)CC3)cccc2c1)c1ncco1] |
| | 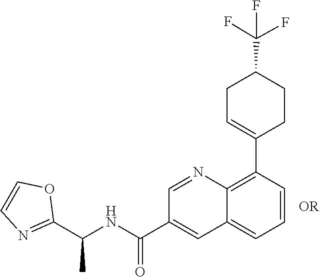 [SMILES: C[C@H](NC(=O)c1cnc2c(C3=CC[C@H](C(F)(F)F)CC3)cccc2c1)c1ncco1] |
| | 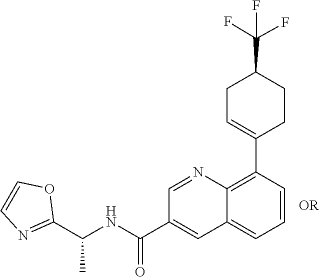 [SMILES: C[C@@H](NC(=O)c1cnc2c(C3=CC[C@@H](C(F)(F)F)CC3)cccc2c1)c1ncco1] |
| | 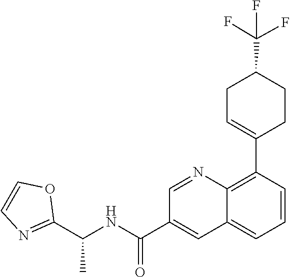 [SMILES: C[C@@H](NC(=O)c1cnc2c(C3=CC[C@H](C(F)(F)F)CC3)cccc2c1)c1ncco1] |
| 20 | 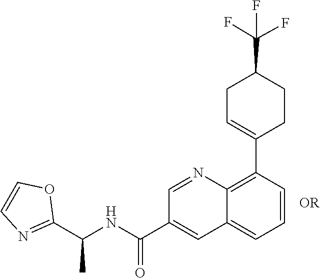 [SMILES: C[C@H](NC(=O)c1cnc2c(C3=CC[C@@H](C(F)(F)F)CC3)cccc2c1)c1ncco1] |
| | 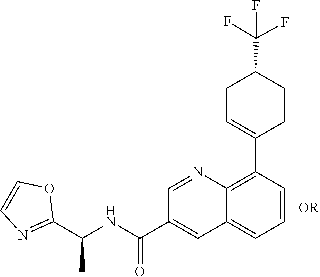 [SMILES: C[C@H](NC(=O)c1cnc2c(C3=CC[C@H](C(F)(F)F)CC3)cccc2c1)c1ncco1] |
| | 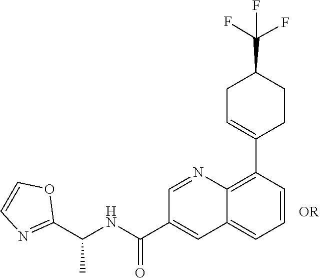 [SMILES: C[C@@H](NC(=O)c1cnc2c(C3=CC[C@@H](C(F)(F)F)CC3)cccc2c1)c1ncco1] |
| | 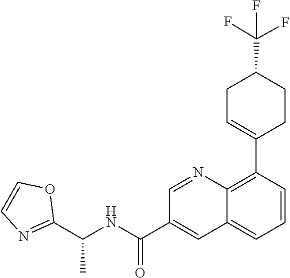 [SMILES: C[C@@H](NC(=O)c1cnc2c(C3=CC[C@H](C(F)(F)F)CC3)cccc2c1)c1ncco1] |
| 21 | 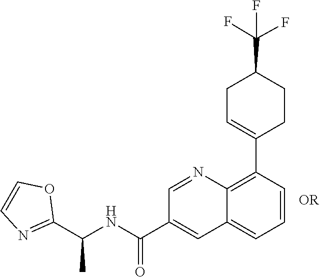 [SMILES: C[C@H](NC(=O)c1cnc2c(C3=CC[C@@H](C(F)(F)F)CC3)cccc2c1)c1ncco1] |
| | 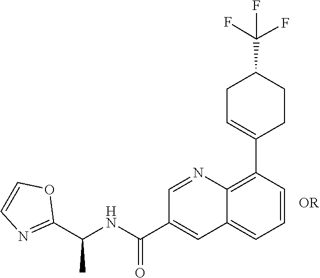 [SMILES: C[C@H](NC(=O)c1cnc2c(C3=CC[C@H](C(F)(F)F)CC3)cccc2c1)c1ncco1] |
| | 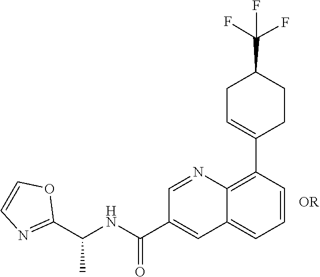 [SMILES: C[C@@H](NC(=O)c1cnc2c(C3=CC[C@@H](C(F)(F)F)CC3)cccc2c1)c1ncco1] |
| | 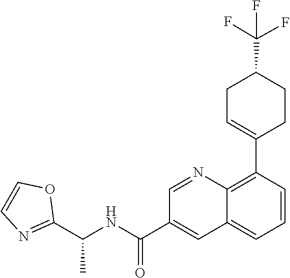 [SMILES: C[C@@H](NC(=O)c1cnc2c(C3=CC[C@H](C(F)(F)F)CC3)cccc2c1)c1ncco1] |
| 22 | 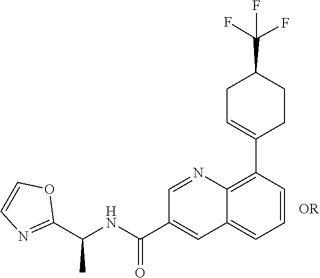 [SMILES: C[C@H](NC(=O)c1cnc2c(C3=CC[C@@H](C(F)(F)F)CC3)cccc2c1)c1ncco1] |
| | 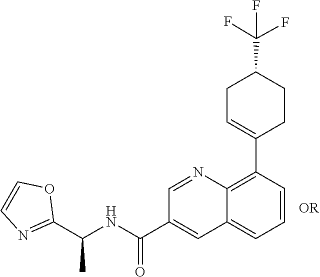 [SMILES: C[C@H](NC(=O)c1cnc2c(C3=CC[C@H](C(F)(F)F)CC3)cccc2c1)c1ncco1] |
| | 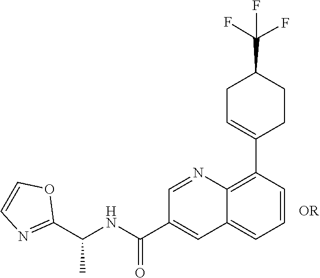 [SMILES: C[C@@H](NC(=O)c1cnc2c(C3=CC[C@@H](C(F)(F)F)CC3)cccc2c1)c1ncco1] |
| | 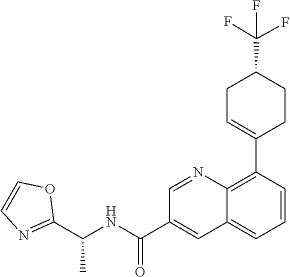 [SMILES: C[C@@H](NC(=O)c1cnc2c(C3=CC[C@H](C(F)(F)F)CC3)cccc2c1)c1ncco1] |
| 23 | 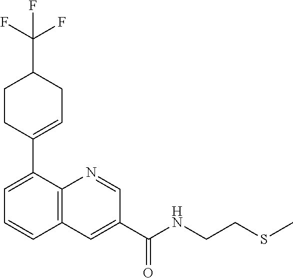 [SMILES: CSCCNC(=O)c1cnc2c(C3=CCC(C(F)(F)F)CC3)cccc2c1] |
| 24 | 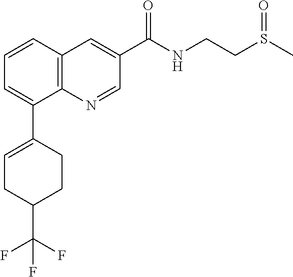 [SMILES: CS(=O)CCNC(=O)c1cnc2c(C3=CCC(C(F)(F)F)CC3)cccc2c1] |
| 25 | 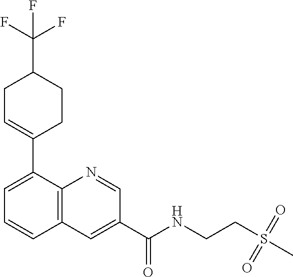 [SMILES: CS(=O)(=O)CCNC(=O)c1cnc2c(C3=CCC(C(F)(F)F)CC3)cccc2c1] |
| 26 | 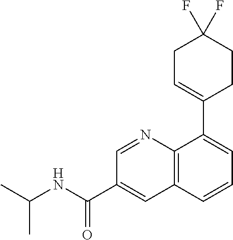 [SMILES: CC(C)NC(=O)c1cnc2c(C3=CCC(F)(F)CC3)cccc2c1] |
| 27 | 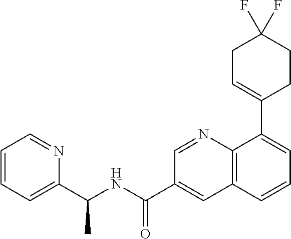 [SMILES: C[C@H](NC(=O)c1cnc2c(C3=CCC(F)(F)CC3)cccc2c1)c1ccccn1] |
| 28 | 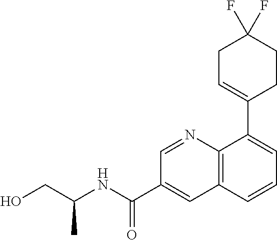 [SMILES: C[C@@H](CO)NC(=O)c1cnc2c(C3=CCC(F)(F)CC3)cccc2c1] |
| 29 | 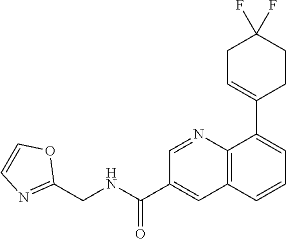 [SMILES: O=C(NCc1ncco1)c1cnc2c(C3=CCC(F)(F)CC3)cccc2c1] |
| 30 | 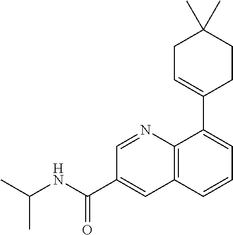 [SMILES: CC(C)NC(=O)c1cnc2c(C3=CCC(C)(C)CC3)cccc2c1] |
| 31 | 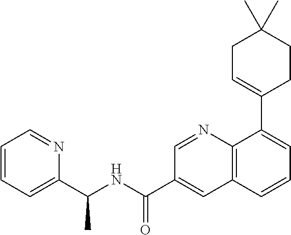 [SMILES: C[C@H](NC(=O)c1cnc2c(C3=CCC(C)(C)CC3)cccc2c1)c1ccccn1] |
| 32 | 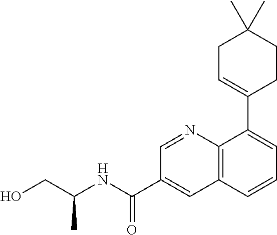 [SMILES: C[C@@H](CO)NC(=O)c1cnc2c(C3=CCC(C)(C)CC3)cccc2c1] |
| 33 | 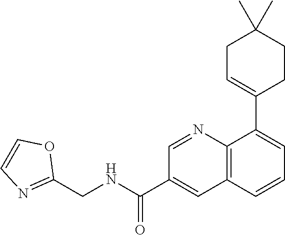 [SMILES: CC1(C)CC=C(c2cccc3cc(C(=O)NCc4ncco4)cnc23)CC1] |
| 34 | 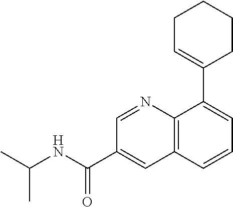 [SMILES: CC(C)NC(=O)c1cnc2c(C3=CCCCC3)cccc2c1] |
| 35 | 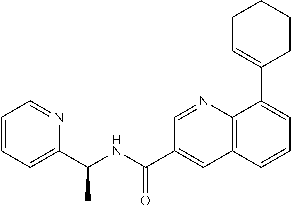 [SMILES: C[C@H](NC(=O)c1cnc2c(C3=CCCCC3)cccc2c1)c1ccccn1] |
| 36 | 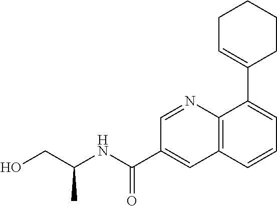 [SMILES: C[C@@H](CO)NC(=O)c1cnc2c(C3=CCCCC3)cccc2c1] |
| 37 | 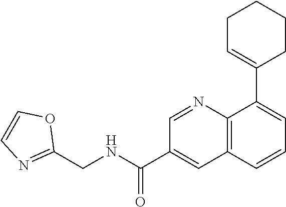 [SMILES: O=C(NCc1ncco1)c1cnc2c(C3=CCCCC3)cccc2c1] |
| 38 | 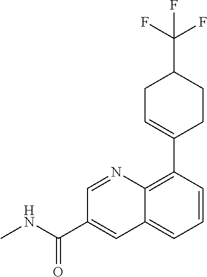 [SMILES: CNC(=O)c1cnc2c(C3=CCC(C(F)(F)F)CC3)cccc2c1] |
| 39 | 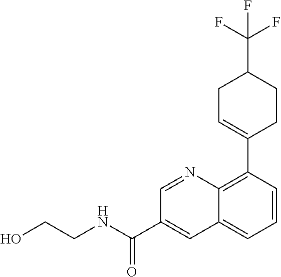 [SMILES: O=C(NCCO)c1cnc2c(C3=CCC(C(F)(F)F)CC3)cccc2c1] |
| 40 | 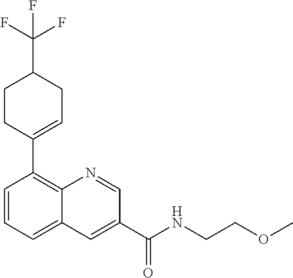 [SMILES: COCCNC(=O)c1cnc2c(C3=CCC(C(F)(F)F)CC3)cccc2c1] |
| 41 | 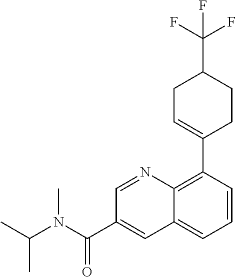 [SMILES: CC(C)N(C)C(=O)c1cnc2c(C3=CCC(C(F)(F)F)CC3)cccc2c1] |
| 42 | 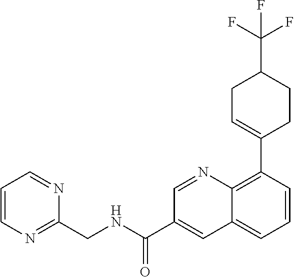 [SMILES: O=C(NCc1ncccn1)c1cnc2c(C3=CCC(C(F)(F)F)CC3)cccc2c1] |
| 43 | 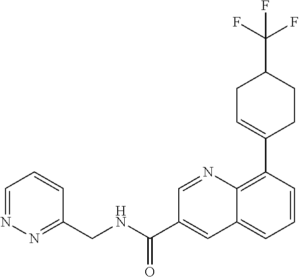 [SMILES: O=C(NCc1cccnn1)c1cnc2c(C3=CCC(C(F)(F)F)CC3)cccc2c1] |
| 44 | 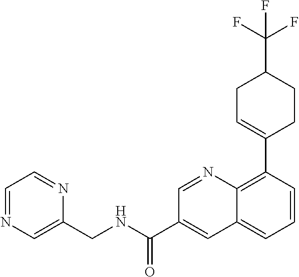 [SMILES: O=C(NCc1cnccn1)c1cnc2c(C3=CCC(C(F)(F)F)CC3)cccc2c1] |
| 45 | 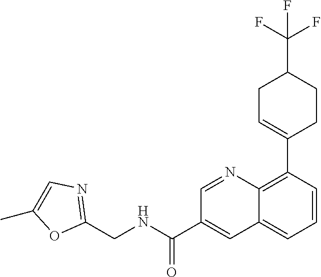 [SMILES: Cc1cnc(CNC(=O)c2cnc3c(C4=CCC(C(F)(F)F)CC4)cccc3c2)o1] |
| 46 | 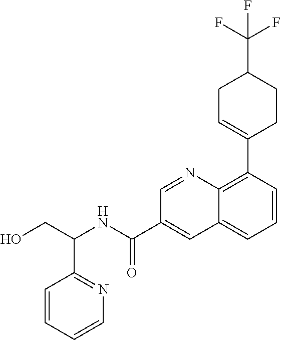 [SMILES: O=C(NC(CO)c1ccccn1)c1cnc2c(C3=CCC(C(F)(F)F)CC3)cccc2c1] |
| 47 | 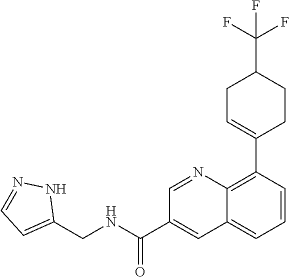 [SMILES: O=C(NCc1ccn[nH]1)c1cnc2c(C3=CCC(C(F)(F)F)CC3)cccc2c1] |
| 48 | 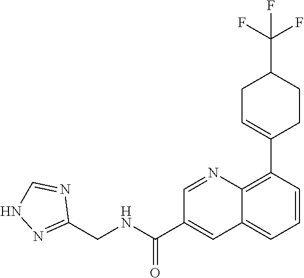 [SMILES: O=C(NCc1nc[nH]n1)c1cnc2c(C3=CCC(C(F)(F)F)CC3)cccc2c1] |
| 49 | 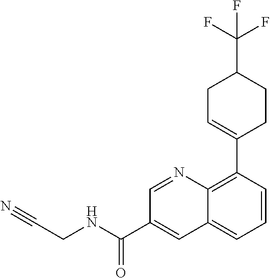 [SMILES: N#CCNC(=O)c1cnc2c(C3=CCC(C(F)(F)F)CC3)cccc2c1] |
| 50 | 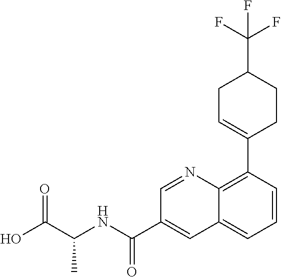 [SMILES: C[C@@H](NC(=O)c1cnc2c(C3=CCC(C(F)(F)F)CC3)cccc2c1)C(=O)O] |
| 51 | 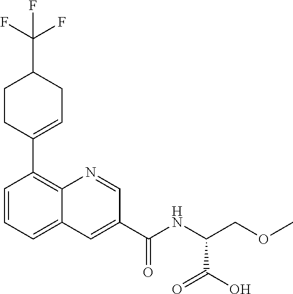 [SMILES: COC[C@@H](NC(=O)c1cnc2c(C3=CCC(C(F)(F)F)CC3)cccc2c1)C(=O)O] |
| 52 | 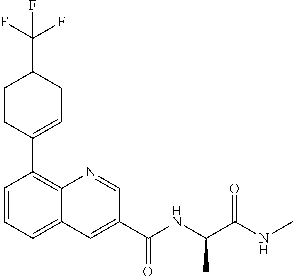 [SMILES: CNC(=O)[C@@H](C)NC(=O)c1cnc2c(C3=CCC(C(F)(F)F)CC3)cccc2c1] |
| 53 | 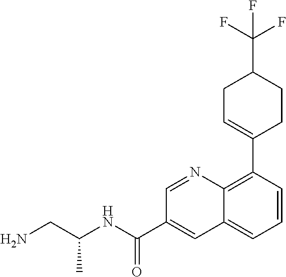 [SMILES: C[C@H](CN)NC(=O)c1cnc2c(C3=CCC(C(F)(F)F)CC3)cccc2c1] |
| 54 | 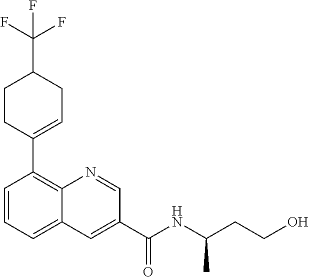 [SMILES: C[C@H](CCO)NC(=O)c1cnc2c(C3=CCC(C(F)(F)F)CC3)cccc2c1] |
| 55 | 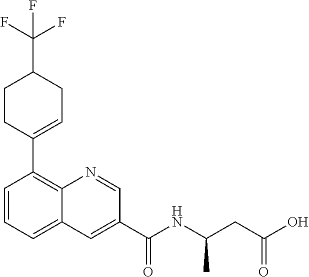 [SMILES: C[C@H](CC(=O)O)NC(=O)c1cnc2c(C3=CCC(C(F)(F)F)CC3)cccc2c1] |
| 56 | 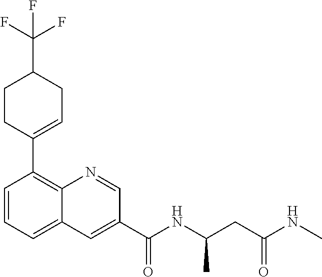 [SMILES: CNC(=O)C[C@@H](C)NC(=O)c1cnc2c(C3=CCC(C(F)(F)F)CC3)cccc2c1] |
| 57 | 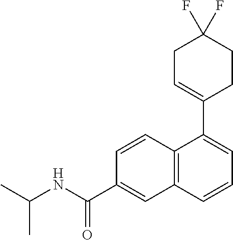 [SMILES: CC(C)NC(=O)c1ccc2c(C3=CCC(F)(F)CC3)cccc2c1] |
| 58 | 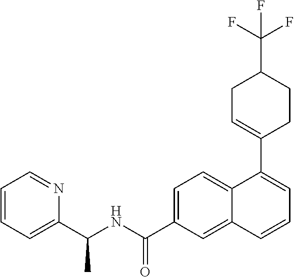 [SMILES: C[C@H](NC(=O)c1ccc2c(C3=CCC(C(F)(F)F)CC3)cccc2c1)c1ccccn1] |
| 59 | 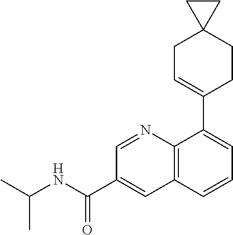 [SMILES: CC(C)NC(=O)c1cnc2c(C3=CCC4(CC3)CC4)cccc2c1] |
| 60 | 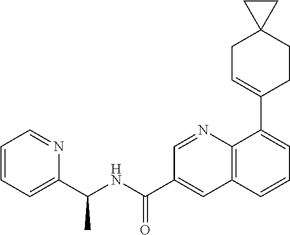 [SMILES: C[C@H](NC(=O)c1cnc2c(C3=CCC4(CC3)CC4)cccc2c1)c1ccccn1] |
| 61 | 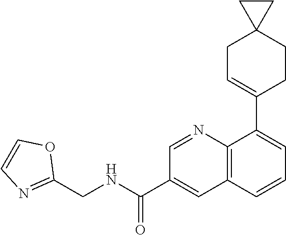 [SMILES: O=C(NCc1ncco1)c1cnc2c(C3=CCC4(CC3)CC4)cccc2c1] |
| 62 | 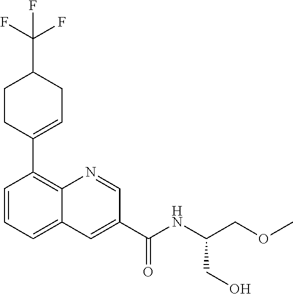 [SMILES: COC[C@H](CO)NC(=O)c1cnc2c(C3=CCC(C(F)(F)F)CC3)cccc2c1] |
| 63 | 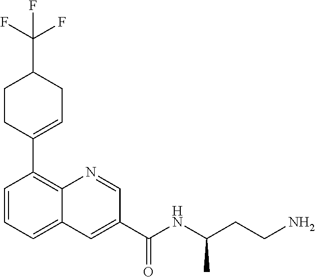 [SMILES: C[C@H](CCN)NC(=O)c1cnc2c(C3=CCC(C(F)(F)F)CC3)cccc2c1] |
| 64 | 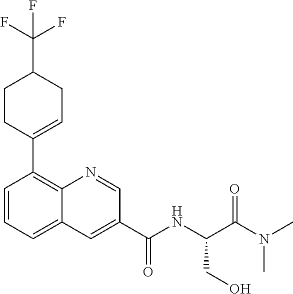 [SMILES: CN(C)C(=O)[C@H](CO)NC(=O)c1cnc2c(C3=CCC(C(F)(F)F)CC3)cccc2c1] |
| 65 | 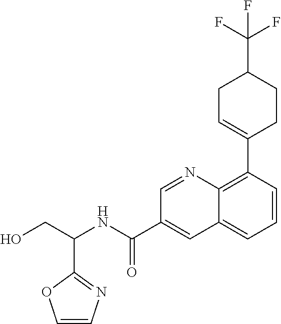 [SMILES: O=C(NC(CO)c1ncco1)c1cnc2c(C3=CCC(C(F)(F)F)CC3)cccc2c1] |
| 66 | 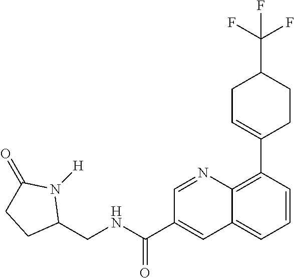 [SMILES: O=C1CCC(CNC(=O)c2cnc3c(C4=CCC(C(F)(F)F)CC4)cccc3c2)N1] |
| 67 | 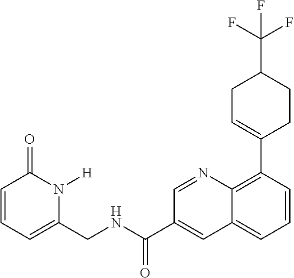 [SMILES: O=C(NCc1cccc(=O)[nH]1)c1cnc2c(C3=CCC(C(F)(F)F)CC3)cccc2c1] |
| 68 | 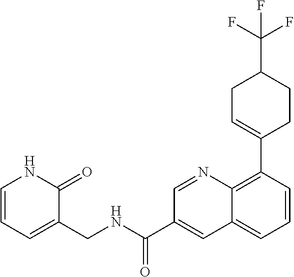 [SMILES: O=C(NCc1ccc[nH]c1=O)c1cnc2c(C3=CCC(C(F)(F)F)CC3)cccc2c1] |
| 69 | 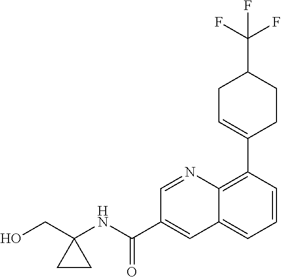 [SMILES: O=C(NC1(CO)CC1)c1cnc2c(C3=CCC(C(F)(F)F)CC3)cccc2c1] |
| 70 | 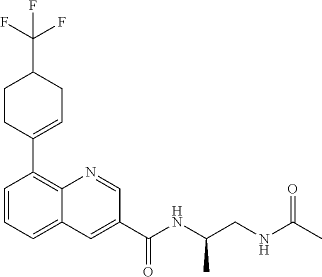 [SMILES: CC(=O)NC[C@@H](C)NC(=O)c1cnc2c(C3=CCC(C(F)(F)F)CC3)cccc2c1] |
| 71 | 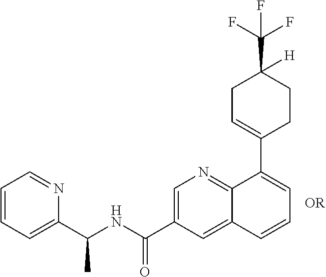 [SMILES: C[C@H](NC(=O)c1cnc2c(C3=CC[C@@H](C(F)(F)F)CC3)cccc2c1)c1ccccn1] |
| | 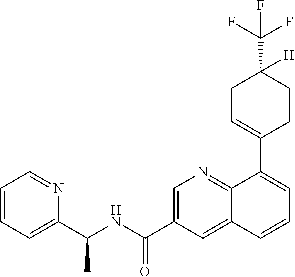 [SMILES: C[C@H](NC(=O)c1cnc2c(C3=CC[C@H](C(F)(F)F)CC3)cccc2c1)c1ccccn1] |
| 72 | 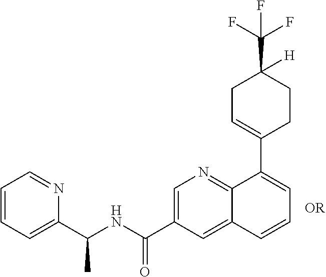 [SMILES: C[C@H](NC(=O)c1cnc2c(C3=CC[C@@H](C(F)(F)F)CC3)cccc2c1)c1ccccn1] |
| | 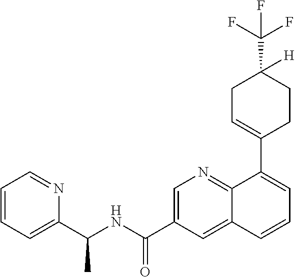 [SMILES: C[C@H](NC(=O)c1cnc2c(C3=CC[C@H](C(F)(F)F)CC3)cccc2c1)c1ccccn1] |
| 73 | 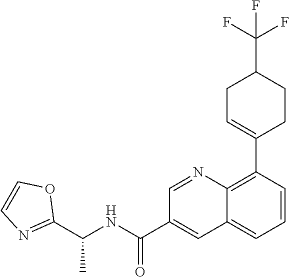 [SMILES: C[C@@H](NC(=O)c1cnc2c(C3=CCC(C(F)(F)F)CC3)cccc2c1)c1ncco1] |
| 74 | 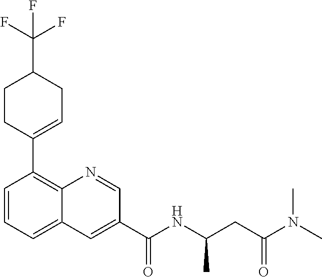 [SMILES: C[C@H](CC(=O)N(C)C)NC(=O)c1cnc2c(C3=CCC(C(F)(F)F)CC3)cccc2c1] |
| 75 | 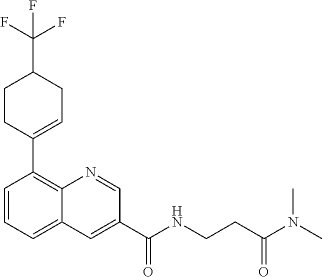 [SMILES: CN(C)C(=O)CCNC(=O)c1cnc2c(C3=CCC(C(F)(F)F)CC3)cccc2c1] |
| 76 | 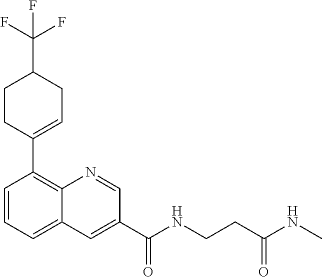 [SMILES: CNC(=O)CCNC(=O)c1cnc2c(C3=CCC(C(F)(F)F)CC3)cccc2c1] |
| 77 | 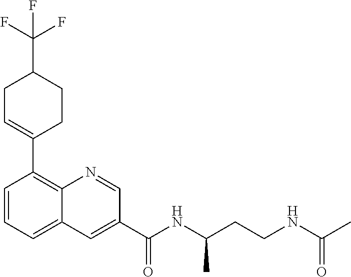 [SMILES: CC(=O)NCC[C@@H](C)NC(=O)c1cnc2c(C3=CCC(C(F)(F)F)CC3)cccc2c1] |
| 78 | 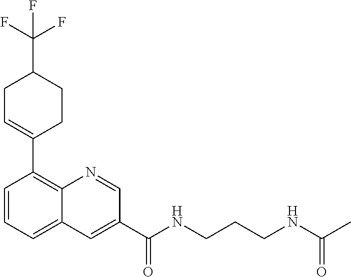 [SMILES: CC(=O)NCCCNC(=O)c1cnc2c(C3=CCC(C(F)(F)F)CC3)cccc2c1] |
| 79 | 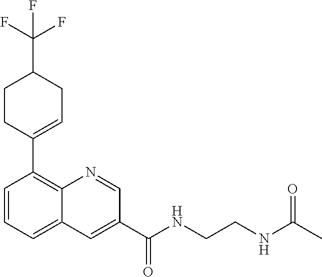 [SMILES: CC(=O)NCCNC(=O)c1cnc2c(C3=CCC(C(F)(F)F)CC3)cccc2c1] |
| 80 | 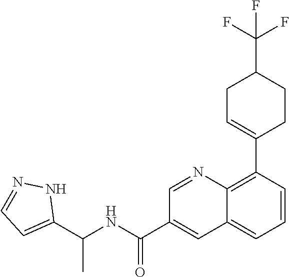 [SMILES: CC(NC(=O)c1cnc2c(C3=CCC(C(F)(F)F)CC3)cccc2c1)c1ccn[nH]1] |
| 81 | 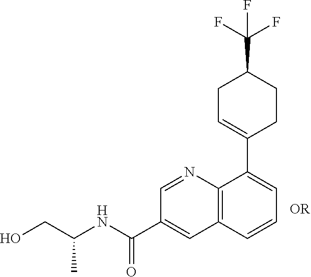 [SMILES: C[C@H](CO)NC(=O)c1cnc2c(C3=CC[C@@H](C(F)(F)F)CC3)cccc2c1] |
| | 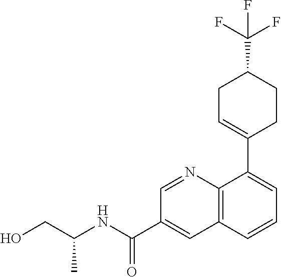 [SMILES: C[C@H](CO)NC(=O)c1cnc2c(C3=CC[C@H](C(F)(F)F)CC3)cccc2c1] |
| 82 | 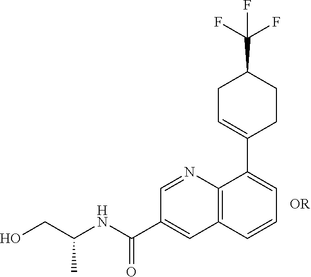 [SMILES: C[C@H](CO)NC(=O)c1cnc2c(C3=CC[C@@H](C(F)(F)F)CC3)cccc2c1] |
| | 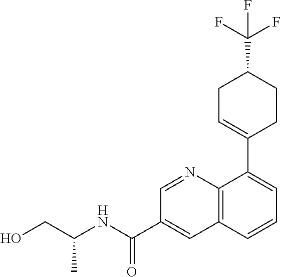 [SMILES: C[C@H](CO)NC(=O)c1cnc2c(C3=CC[C@H](C(F)(F)F)CC3)cccc2c1] |
| 83 | 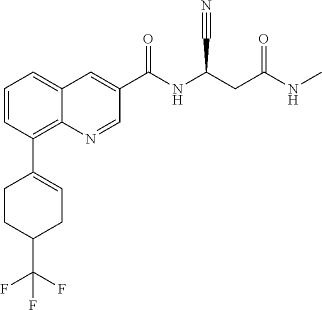 [SMILES: CNC(=O)C[C@H](C#N)NC(=O)c1cnc2c(C3=CCC(C(F)(F)F)CC3)cccc2c1] |
| 84 | 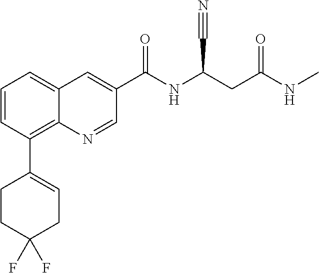 [SMILES: CNC(=O)C[C@H](C#N)NC(=O)c1cnc2c(C3=CCC(F)(F)CC3)cccc2c1] |
| 85 | 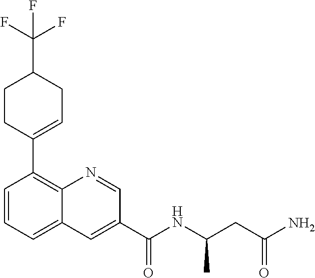 [SMILES: C[C@H](CC(N)=O)NC(=O)c1cnc2c(C3=CCC(C(F)(F)F)CC3)cccc2c1] |
| 86 | 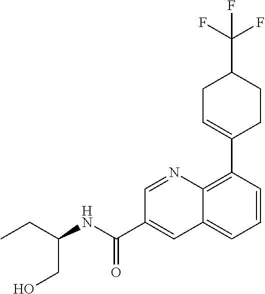 [SMILES: CC[C@H](CO)NC(=O)c1cnc2c(C3=CCC(C(F)(F)F)CC3)cccc2c1] |
| 87 | 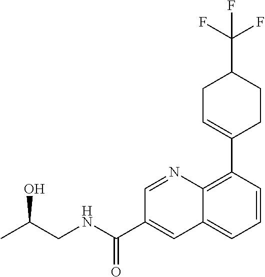 [SMILES: C[C@@H](O)CNC(=O)c1cnc2c(C3=CCC(C(F)(F)F)CC3)cccc2c1] |
| 88 | 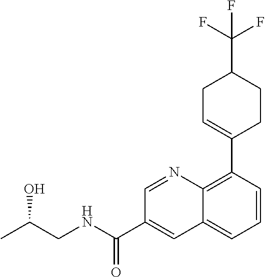 [SMILES: C[C@H](O)CNC(=O)c1cnc2c(C3=CCC(C(F)(F)F)CC3)cccc2c1] |
| 89 | 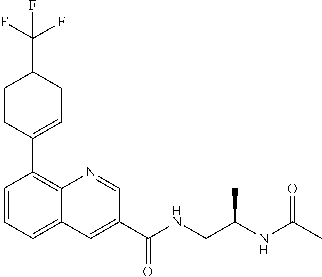 [SMILES: CC(=O)N[C@H](C)CNC(=O)c1cnc2c(C3=CCC(C(F)(F)F)CC3)cccc2c1] |
| 90 | 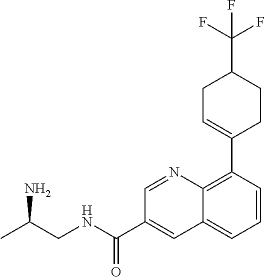 [SMILES: C[C@@H](N)CNC(=O)c1cnc2c(C3=CCC(C(F)(F)F)CC3)cccc2c1] |
| 91 | 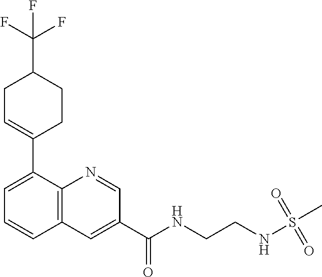 [SMILES: CS(=O)(=O)NCCNC(=O)c1cnc2c(C3=CCC(C(F)(F)F)CC3)cccc2c1] |
| 92 | 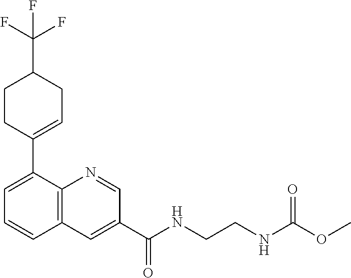 [SMILES: COC(=O)NCCNC(=O)c1cnc2c(C3=CCC(C(F)(F)F)CC3)cccc2c1] |
| 93 | 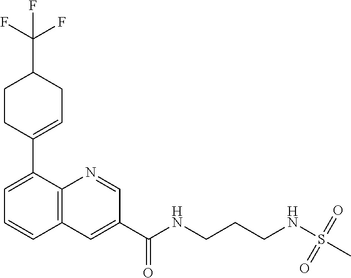 [SMILES: CS(=O)(=O)NCCCNC(=O)c1cnc2c(C3=CCC(C(F)(F)F)CC3)cccc2c1] |
| 94 | 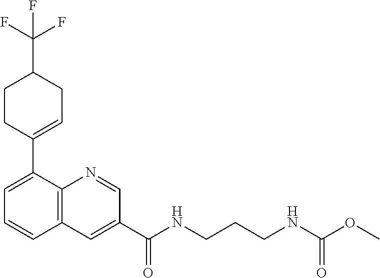 [SMILES: COC(=O)NCCCNC(=O)c1cnc2c(C3=CCC(C(F)(F)F)CC3)cccc2c1] |
| 95 | 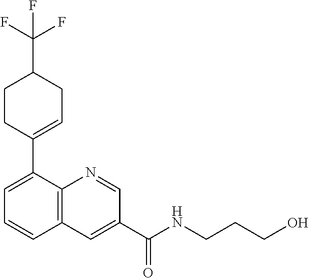 [SMILES: O=C(NCCCO)c1cnc2c(C3=CCC(C(F)(F)F)CC3)cccc2c1] |
| 96 | 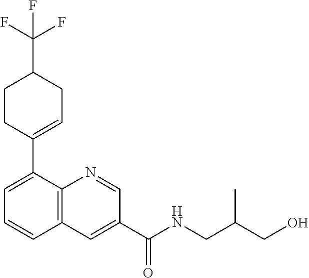 [SMILES: CC(CO)CNC(=O)c1cnc2c(C3=CCC(C(F)(F)F)CC3)cccc2c1] |
| 97 | 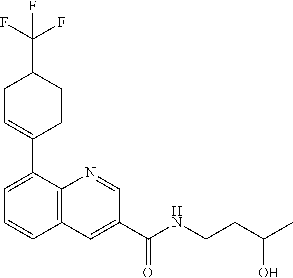 [SMILES: CC(O)CCNC(=O)c1cnc2c(C3=CCC(C(F)(F)F)CC3)cccc2c1] |
| 98 | 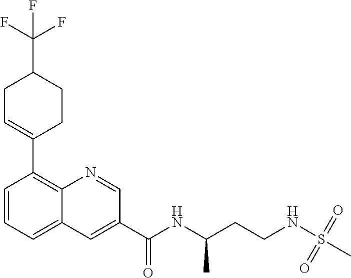 [SMILES: C[C@H](CCNS(C)(=O)=O)NC(=O)c1cnc2c(C3=CCC(C(F)(F)F)CC3)cccc2c1] |
| 99 | 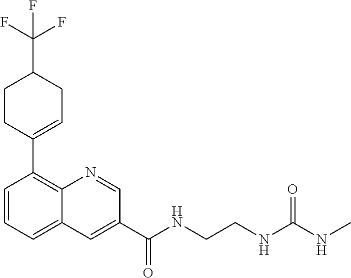 [SMILES: CNC(=O)NCCNC(=O)c1cnc2c(C3=CCC(C(F)(F)F)CC3)cccc2c1] |
| 100 | 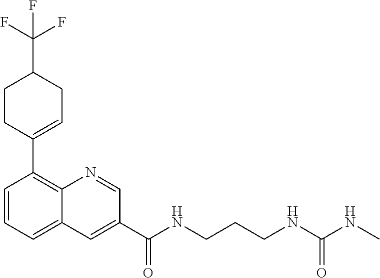 [SMILES: CNC(=O)NCCCNC(=O)c1cnc2c(C3=CCC(C(F)(F)F)CC3)cccc2c1] |
| 101 | 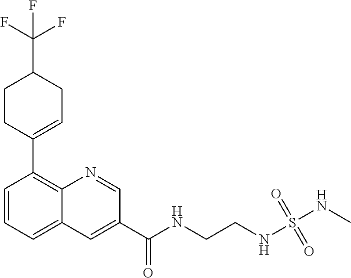 [SMILES: CNS(=O)(=O)NCCNC(=O)c1cnc2c(C3=CCC(C(F)(F)F)CC3)cccc2c1] |
| 102 | 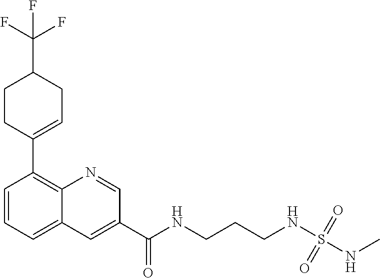 [SMILES: CNS(=O)(=O)NCCCNC(=O)c1cnc2c(C3=CCC(C(F)(F)F)CC3)cccc2c1] |
| 103 | 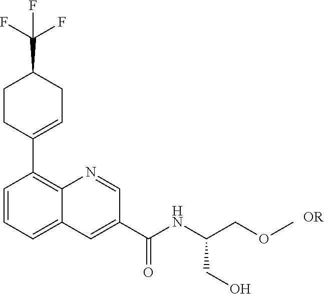 [SMILES: COC[C@H](CO)NC(=O)c1cnc2c(C3=CC[C@H](C(F)(F)F)CC3)cccc2c1] |
| | 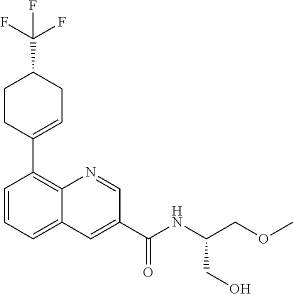 [SMILES: COC[C@H](CO)NC(=O)c1cnc2c(C3=CC[C@@H](C(F)(F)F)CC3)cccc2c1] |
| 104 | 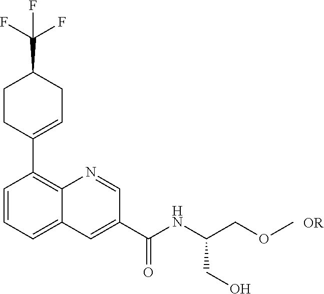 [SMILES: COC[C@H](CO)NC(=O)c1cnc2c(C3=CC[C@H](C(F)(F)F)CC3)cccc2c1] |
| | 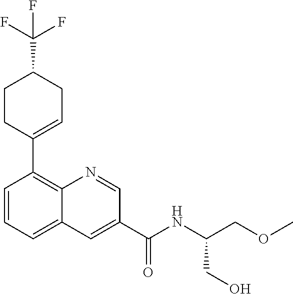 [SMILES: COC[C@H](CO)NC(=O)c1cnc2c(C3=CC[C@@H](C(F)(F)F)CC3)cccc2c1] |
| 105 | 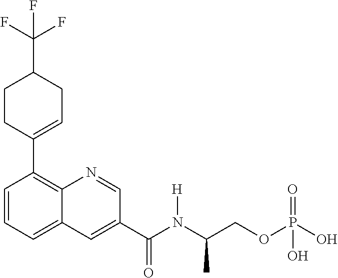 [SMILES: C[C@H](COP(=O)(O)O)NC(=O)c1cnc2c(C3=CCC(C(F)(F)F)CC3)cccc2c1] |
| 106 | 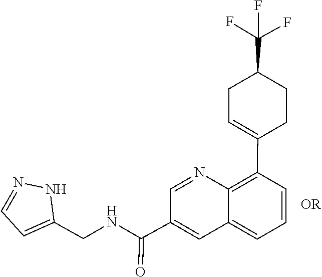 [SMILES: O=C(NCc1ccn[nH]1)c1cnc2c(C3=CC[C@@H](C(F)(F)F)CC3)cccc2c1] |
| | 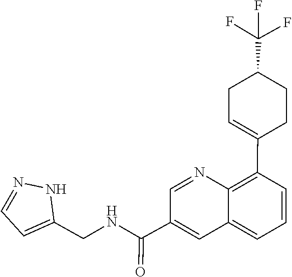 [SMILES: O=C(NCc1ccn[nH]1)c1cnc2c(C3=CC[C@H](C(F)(F)F)CC3)cccc2c1] |
| 107 | 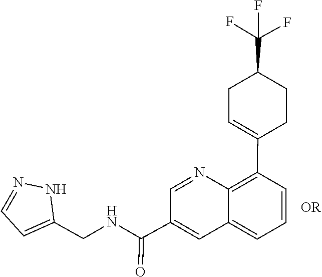 [SMILES: O=C(NCc1ccn[nH]1)c1cnc2c(C3=CC[C@@H](C(F)(F)F)CC3)cccc2c1] |
| | 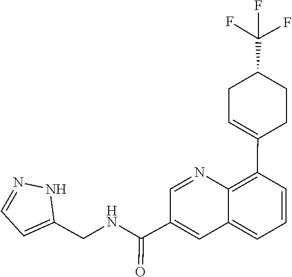 [SMILES: O=C(NCc1ccn[nH]1)c1cnc2c(C3=CC[C@H](C(F)(F)F)CC3)cccc2c1] |
| 108 | 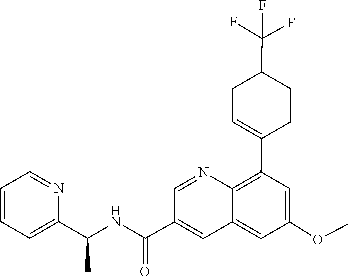 [SMILES: COc1cc(C2=CCC(C(F)(F)F)CC2)c2ncc(C(=O)N[C@@H](C)c3ccccn3)cc2c1] |
| 109 | 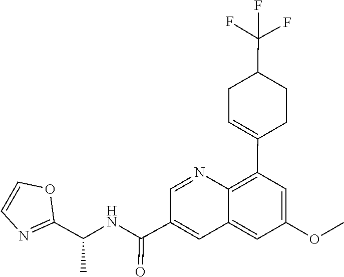 [SMILES: COc1cc(C2=CCC(C(F)(F)F)CC2)c2ncc(C(=O)N[C@H](C)c3ncco3)cc2c1] |
| 110 | 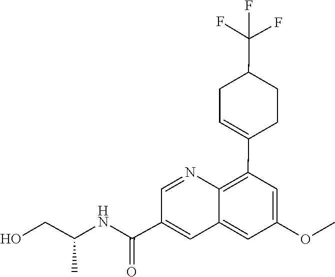 [SMILES: COc1cc(C2=CCC(C(F)(F)F)CC2)c2ncc(C(=O)N[C@H](C)CO)cc2c1] |
| 111 | 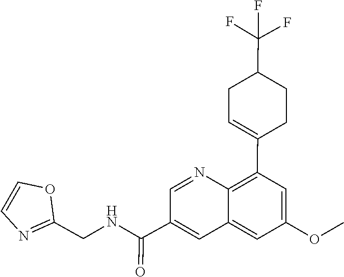 [SMILES: COc1cc(C2=CCC(C(F)(F)F)CC2)c2ncc(C(=O)NCc3ncco3)cc2c1] |
| 112 | 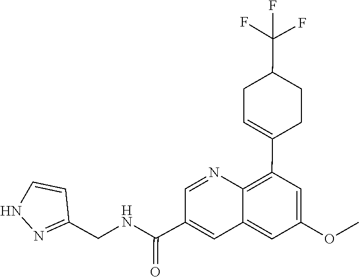 [SMILES: COc1cc(C2=CCC(C(F)(F)F)CC2)c2ncc(C(=O)NCc3cc[nH]n3)cc2c1] |
| 113 | 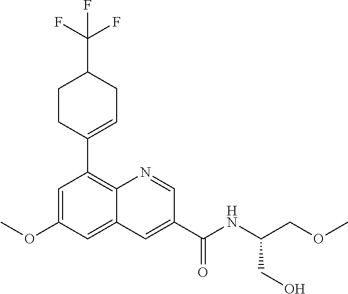 |
| 114 | 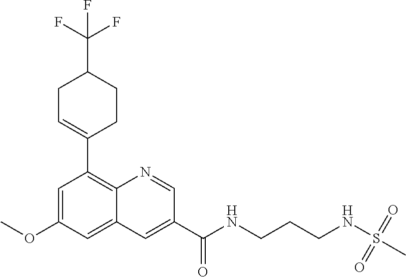 |
| 115 | 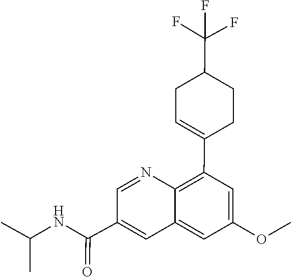 [SMILES: COc1cc(C2=CCC(C(F)(F)F)CC2)c2ncc(C(=O)NC(C)C)cc2c1] |
| 116 | 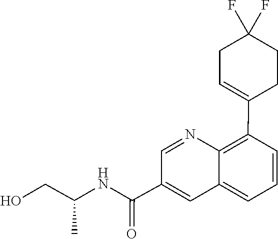 [SMILES: C[C@H](CO)NC(=O)c1cnc2c(C3=CCC(F)(F)CC3)cccc2c1] |
| 117 | 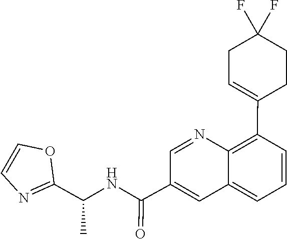 [SMILES: C[C@@H](NC(=O)c1cnc2c(C3=CCC(F)(F)CC3)cccc2c1)c1ncco1] |
| 118 | 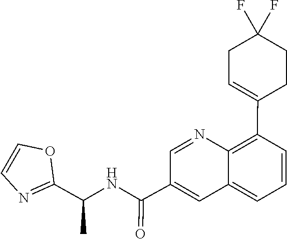 [SMILES: C[C@H](NC(=O)c1cnc2c(C3=CCC(F)(F)CC3)cccc2c1)c1ncco1] |
| 119 | 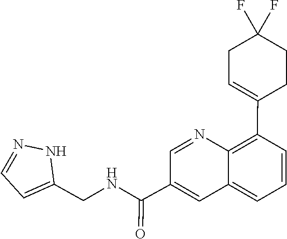 [SMILES: O=C(NCc1ccn[nH]1)c1cnc2c(C3=CCC(F)(F)CC3)cccc2c1] |
| 120 | 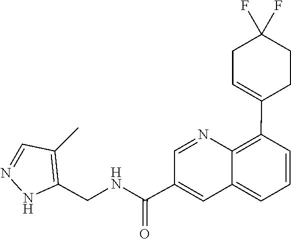 [SMILES: Cc1cn[nH]c1CNC(=O)c1cnc2c(C3=CCC(F)(F)CC3)cccc2c1] |
| 121 | 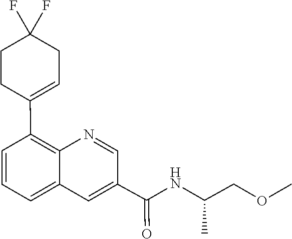 [SMILES: COC[C@H](C)NC(=O)c1cnc2c(C3=CCC(F)(F)CC3)cccc2c1] |
| 122 | 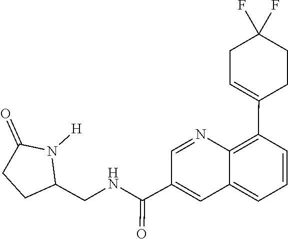 [SMILES: O=C1CCC(CNC(=O)c2cnc3c(C4=CCC(F)(F)CC4)cccc3c2)N1] |
| 123 | 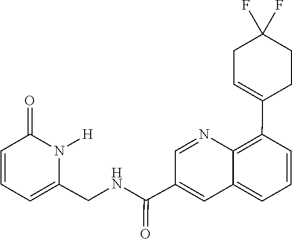 [SMILES: O=C(NCc1cccc(=O)[nH]1)c1cnc2c(C3=CCC(F)(F)CC3)cccc2c1] |
| 124 | 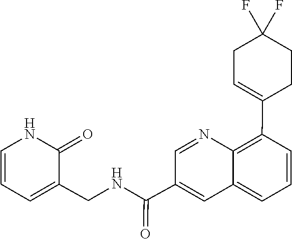 [SMILES: O=C(NCc1ccc[nH]c1=O)c1cnc2c(C3=CCC(F)(F)CC3)cccc2c1] |
| 125 | 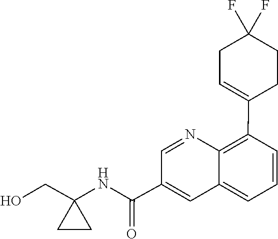 [SMILES: O=C(NC1(CO)CC1)c1cnc2c(C3=CCC(F)(F)CC3)cccc2c1] |
| 126 | 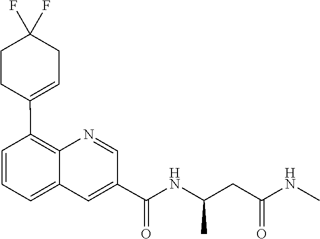 [SMILES: CNC(=O)C[C@@H](C)NC(=O)c1cnc2c(C3=CCC(F)(F)CC3)cccc2c1] |
| 127 | 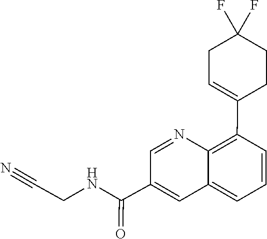 [SMILES: N#CCNC(=O)c1cnc2c(C3=CCC(F)(F)CC3)cccc2c1] |
| 128 | 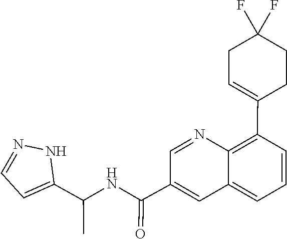 [SMILES: CC(NC(=O)c1cnc2c(C3=CCC(F)(F)CC3)cccc2c1)c1ccn[nH]1] |
| 129 | 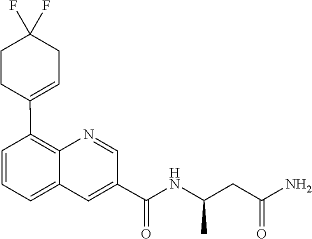 [SMILES: C[C@H](CC(N)=O)NC(=O)c1cnc2c(C3=CCC(F)(F)CC3)cccc2c1] |
| 130 | 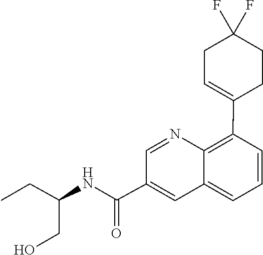 [SMILES: CC[C@H](CO)NC(=O)c1cnc2c(C3=CCC(F)(F)CC3)cccc2c1] |
| 131 | 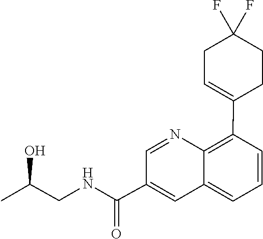 [SMILES: C[C@@H](O)CNC(=O)c1cnc2c(C3=CCC(F)(F)CC3)cccc2c1] |
| 132 | 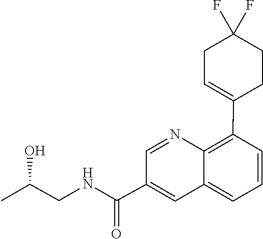 [SMILES: C[C@H](O)CNC(=O)c1cnc2c(C3=CCC(F)(F)CC3)cccc2c1] |
| 133 | 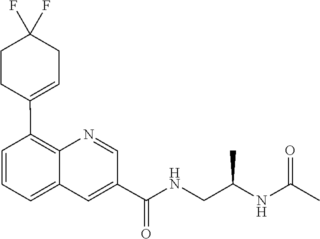 [SMILES: CC(=O)N[C@H](C)CNC(=O)c1cnc2c(C3=CCC(F)(F)CC3)cccc2c1] |
| 134 | 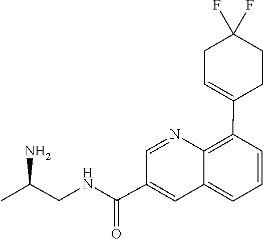 [SMILES: C[C@@H](N)CNC(=O)c1cnc2c(C3=CCC(F)(F)CC3)cccc2c1] |
| 135 | 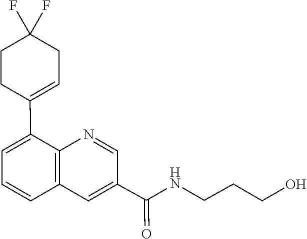 [SMILES: O=C(NCCCO)c1cnc2c(C3=CCC(F)(F)CC3)cccc2c1] |
| 136 | 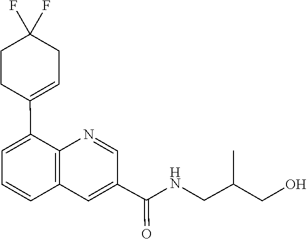 [SMILES: CC(CO)CNC(=O)c1cnc2c(C3=CCC(F)(F)CC3)cccc2c1] |
| 137 | 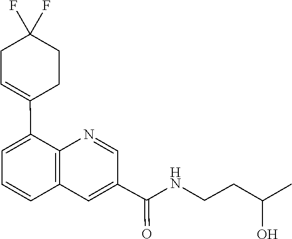 [SMILES: CC(O)CCNC(=O)c1cnc2c(C3=CCC(F)(F)CC3)cccc2c1] |
| 138 | 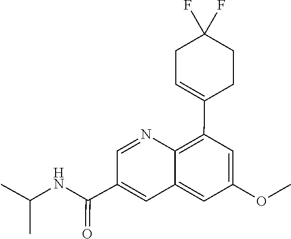 [SMILES: COc1cc(C2=CCC(F)(F)CC2)c2ncc(C(=O)NC(C)C)cc2c1] |
| 139 | 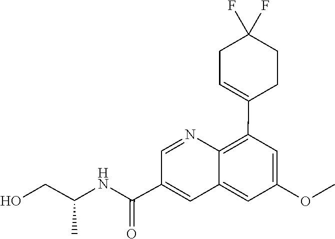 [SMILES: COc1cc(C2=CCC(F)(F)CC2)c2ncc(C(=O)N[C@H](C)CO)cc2c1] |
| 140 | 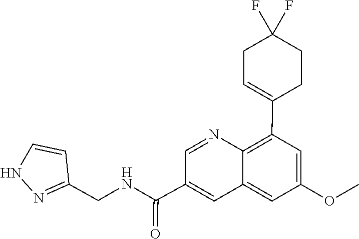 [SMILES: COc1cc(C2=CCC(F)(F)CC2)c2ncc(C(=O)NCc3cc[nH]n3)cc2c1] |
| 141 | 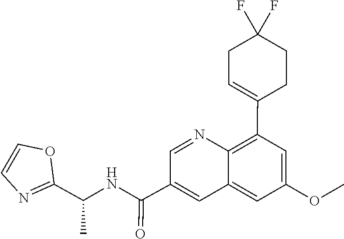 [SMILES: COc1cc(C2=CCC(F)(F)CC2)c2ncc(C(=O)N[C@H](C)c3ncco3)cc2c1] |
| 142 | 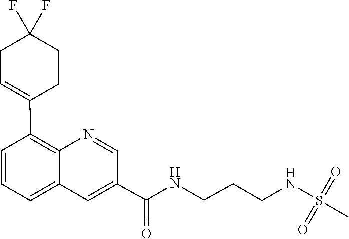 [SMILES: CS(=O)(=O)NCCCNC(=O)c1cnc2c(C3=CCC(F)(F)CC3)cccc2c1] |
| 143 | 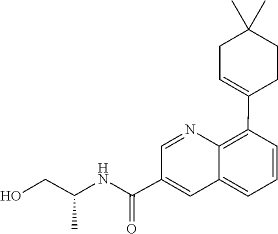 [SMILES: C[C@H](CO)NC(=O)c1cnc2c(C3=CCC(C)(C)CC3)cccc2c1] |
| 144 | 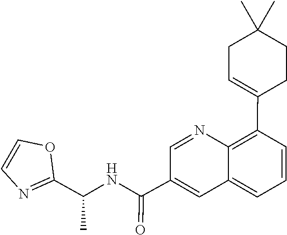 [SMILES: C[C@@H](NC(=O)c1cnc2c(C3=CCC(C)(C)CC3)cccc2c1)c1ncco1] |
| 145 | 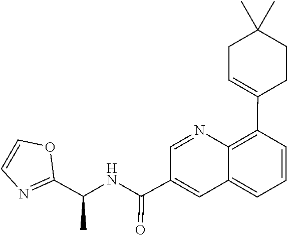 [SMILES: C[C@H](NC(=O)c1cnc2c(C3=CCC(C)(C)CC3)cccc2c1)c1ncco1] |
| 146 | 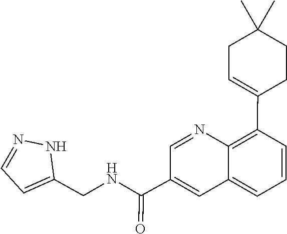 [SMILES: CC1(C)CC=C(c2cccc3cc(C(=O)NCc4ccn[nH]4)cnc23)CC1] |
| 147 | 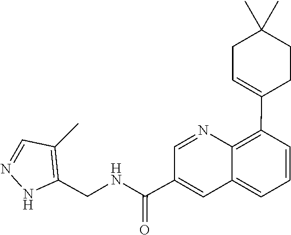 [SMILES: Cc1cn[nH]c1CNC(=O)c1cnc2c(C3=CCC(C)(C)CC3)cccc2c1] |
| 148 | 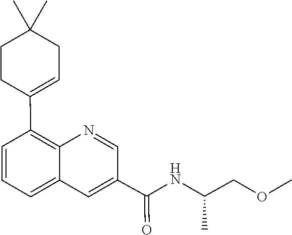 [SMILES: COC[C@H](C)NC(=O)c1cnc2c(C3=CCC(C)(C)CC3)cccc2c1] |
| 149 | 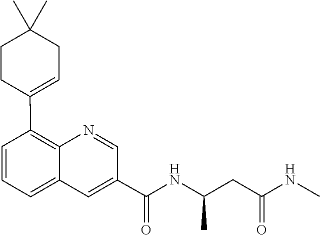 [SMILES: CNC(=O)C[C@@H](C)NC(=O)c1cnc2c(C3=CCC(C)(C)CC3)cccc2c1] |
| 150 | 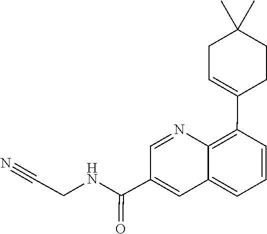 [SMILES: CC1(C)CC=C(c2cccc3cc(C(=O)NCC#N)cnc23)CC1] |
| 151 | 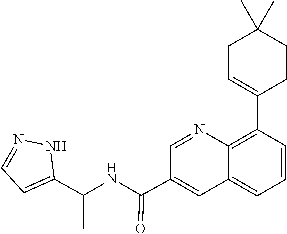 [SMILES: CC(NC(=O)c1cnc2c(C3=CCC(C)(C)CC3)cccc2c1)c1ccn[nH]1] |
| 152 | 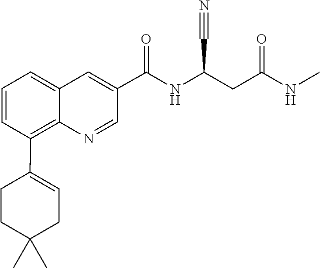 [SMILES: CNC(=O)C[C@H](C#N)NC(=O)c1cnc2c(C3=CCC(C)(C)CC3)cccc2c1] |
| 153 | 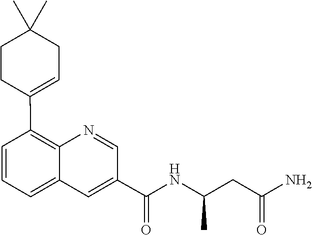 [SMILES: C[C@H](CC(N)=O)NC(=O)c1cnc2c(C3=CCC(C)(C)CC3)cccc2c1] |
| 154 | 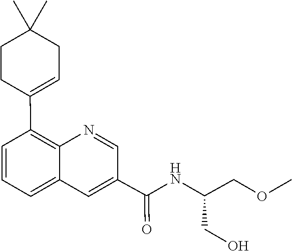 [SMILES: COC[C@H](CO)NC(=O)c1cnc2c(C3=CCC(C)(C)CC3)cccc2c1] |
| 155 | 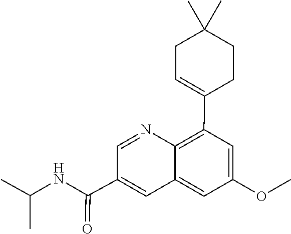 [SMILES: COc1cc(C2=CCC(C)(C)CC2)c2ncc(C(=O)NC(C)C)cc2c1] |
| 156 | 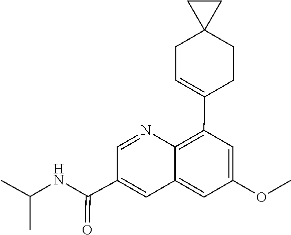 [SMILES: COc1cc(C2=CCC3(CC2)CC3)c2ncc(C(=O)NC(C)C)cc2c1] |
| 157 | 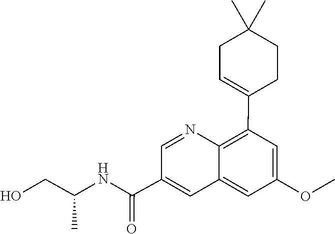 [SMILES: COc1cc(C2=CCC(C)(C)CC2)c2ncc(C(=O)N[C@H](C)CO)cc2c1] |
| 158 | 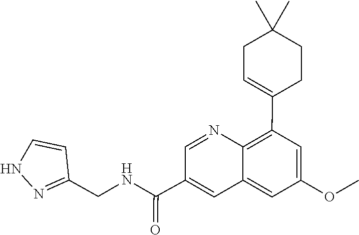 [SMILES: COc1cc(C2=CCC(C)(C)CC2)c2ncc(C(=O)NCc3cc[nH]n3)cc2c1] |
| 159 | 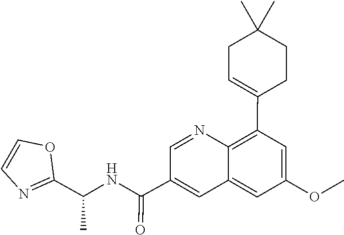 [SMILES: COc1cc(C2=CCC(C)(C)CC2)c2ncc(C(=O)N[C@H](C)c3ncco3)cc2c1] |
| 160 | 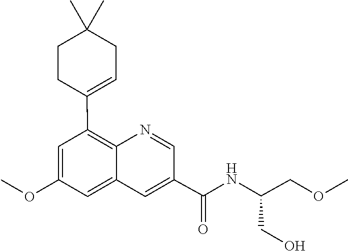 |
| 161 | 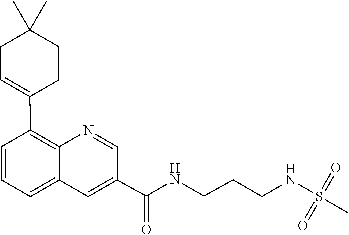 [SMILES: CC1(C)CC=C(c2cccc3cc(C(=O)NCCCNS(C)(=O)=O)cnc23)CC1] |
| 162 | 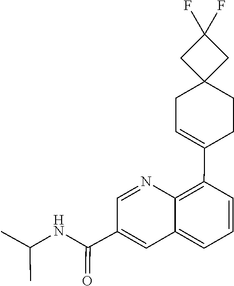 [SMILES: CC(C)NC(=O)c1cnc2c(C3=CCC4(CC3)CC(F)(F)C4)cccc2c1] |
| 163 | 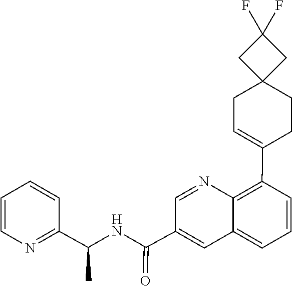 [SMILES: C[C@H](NC(=O)c1cnc2c(C3=CCC4(CC3)CC(F)(F)C4)cccc2c1)c1ccccn1] |
| 164 | 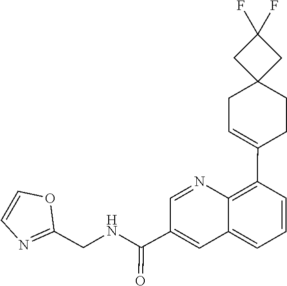 [SMILES: O=C(NCc1ncco1)c1cnc2c(C3=CCC4(CC3)CC(F)(F)C4)cccc2c1] |
| 165 | 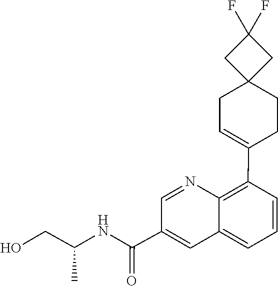 [SMILES: C[C@H](CO)NC(=O)c1cnc2c(C3=CCC4(CC3)CC(F)(F)C4)cccc2c1] |
| 166 | 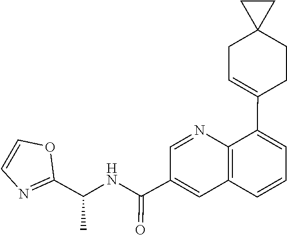 [SMILES: C[C@@H](NC(=O)c1cnc2c(C3=CCC4(CC3)CC4)cccc2c1)c1ncco1] |
| 167 | 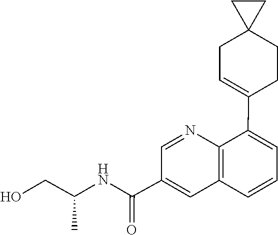 [SMILES: C[C@H](CO)NC(=O)c1cnc2c(C3=CCC4(CC3)CC4)cccc2c1] |
| 168 | 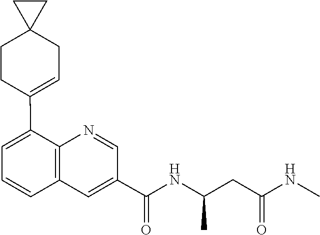 [SMILES: CNC(=O)C[C@@H](C)NC(=O)c1cnc2c(C3=CCC4(CC3)CC4)cccc2c1] |
| 169 | 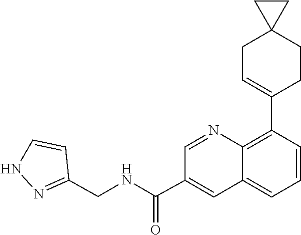 [SMILES: O=C(NCc1cc[nH]n1)c1cnc2c(C3=CCC4(CC3)CC4)cccc2c1] |
| 170 | 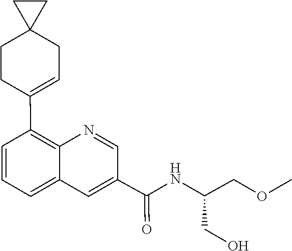 [SMILES: COC[C@H](CO)NC(=O)c1cnc2c(C3=CCC4(CC3)CC4)cccc2c1] |
| 171 | 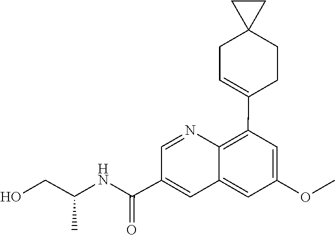 [SMILES: COc1cc(C2=CCC3(CC2)CC3)c2ncc(C(=O)N[C@H](C)CO)cc2c1] |
| 172 | 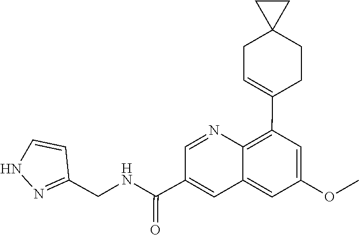 [SMILES: COc1cc(C2=CCC3(CC2)CC3)c2ncc(C(=O)NCc3cc[nH]n3)cc2c1] |
| 173 | 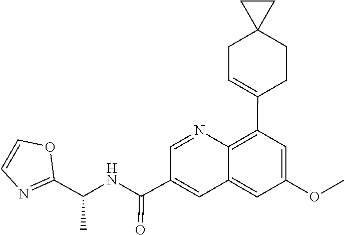 [SMILES: COc1cc(C2=CCC3(CC2)CC3)c2ncc(C(=O)N[C@H](C)c3ncco3)cc2c1] |
| 174 | 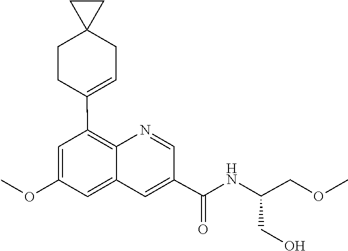 |
| 175 | 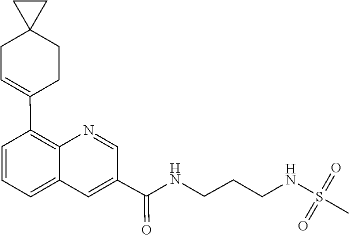 [SMILES: CS(=O)(=O)NCCCNC(=O)c1cnc2c(C3=CCC4(CC3)CC4)cccc2c1] |
| 176 | 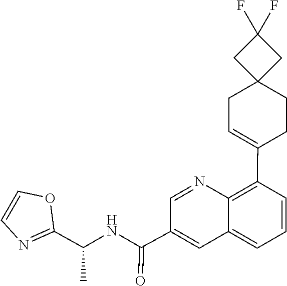 [SMILES: C[C@@H](NC(=O)c1cnc2c(C3=CCC4(CC3)CC(F)(F)C4)cccc2c1)c1ncco1] |
| 177 | 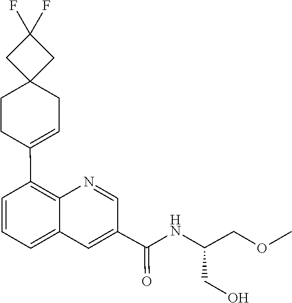 [SMILES: COC[C@H](CO)NC(=O)c1cnc2c(C3=CCC4(CC3)CC(F)(F)C4)cccc2c1] |

### Pharmaceutical Compositions and Routes of Administration

Disclosed herein, in certain embodiments, are pharmaceutical compositions comprising a compound disclosed herein (e.g., a compound of Formula (I), Formula (I′), Formula (Ia), Formula (Ia′), Formula (Ib), Formula (Ic), Formula (Ib-1), or Formula (Ib-2)) or a pharmaceutically acceptable salt or ester thereof, and a pharmaceutically acceptable excipient. In some embodiments, the excipient is selected from: inert solid diluents and fillers, diluents, including sterile aqueous solution and various organic solvents, permeation enhancers, solubilizers and adjuvants. In some embodiments, the pharmaceutical compositions are administered alone or in combination with other therapeutic agents. Such compositions are prepared in a manner well known in the pharmaceutical art.

In some embodiments, the pharmaceutical compositions are administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. In some embodiments, the forms in which the novel compositions of the present disclosure are incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. In some embodiments, ethanol, glycerol, propylene glycol, liquid polyethylene glycol, and the like (and suitable mixtures thereof), cyclodextrin derivatives, and vegetable oils are employed. In some embodiments, the proper fluidity is maintained, for example, by the use of a coating, such as lecithin, by the maintenance of the required particle size in the case of dispersion and by the use of surfactants. In some embodiments, the prevention of the action of microorganisms is brought about by various antibacterial and antifungal agents, for example, parabens, chlorobutanol, phenol, sorbic acid, thimerosal, and the like.

Sterile injectable solutions are prepared by incorporating a compound according to the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the disclosure. In some embodiments, administration is via capsule or enteric coated tablets, or the like. In some embodiments, in making the pharmaceutical compositions that include at least one compound described herein, the active ingredient is usually diluted by an excipient and/or enclosed within such a carrier that is in the form of a capsule, sachet, paper or other container. In some embodiments, when the excipient serves as a diluent, it is in the form of a solid, semi-solid, or liquid material (as above), which acts as a vehicle, carrier or medium for the active ingredient. Thus, in some embodiments, the compositions are e in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. In some embodiments, the formulations additionally include: lubricating agents such as talc, magnesium stearate, and mineral oil; wetting agents; emulsifying and suspending agents; preserving agents such as methyl and propylhydroxy-benzoates; sweetening agents; and flavoring agents.

In some embodiments, the compositions of the disclosure are formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Another formulation for use in the methods of the present disclosure employs transdermal delivery devices (“patches”). In some embodiments, such transdermal patches are used to provide continuous or discontinuous infusion of the compounds of the present disclosure in controlled amounts. The construction and use of transdermal patches for the delivery of pharmaceutical agents is well known in the art. In some embodiments, such patches are constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 2 g of a compound described herein, and for parenteral administration, preferably from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. In some embodiments, when referring to these preformulation compositions as homogeneous, it is meant that the active ingredient is dispersed evenly throughout the composition so that the composition is readily subdivided into equally effective unit dosage forms such as tablets, pills and capsules.

In some embodiments, the tablets or pills of the present disclosure are coated or otherwise compounded to provide a dosage form affording the advantage of prolonged action, or to protect from the acid conditions of the stomach. For example, in some embodiments, the tablet or pill comprises an inner dosage and an outer dosage component, the latter being in the form of an envelope over the former. In some embodiments, the two components are separated by an enteric layer that serves to resist disintegration in the stomach and permit the inner component to pass intact into the duodenum or to be delayed in release. In some embodiments, a variety of materials are used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. In some embodiments, the liquid or solid compositions contain suitable pharmaceutically acceptable excipients as described supra. In some embodiments, the compositions are administered by the oral or nasal respiratory route for local or systemic effect. In some embodiments, compositions in pharmaceutically acceptable solvents are nebulized by use of inert gases. In some embodiments, nebulized solutions are inhaled directly from the nebulizing device or the nebulizing device is attached to a facemask tent, or intermittent positive pressure breathing machine. In some embodiments, solution, suspension, or powder compositions are administered, orally or nasally, from devices that deliver the formulation in an appropriate manner.

### Methods of Treatment

Disclosed herein, in certain embodiments, are methods of treating a disease or disorder modulated by abnormal activity of a TEAD isoform (e.g., TEAD1 and/or TEAD4) (e.g., hyperactivation of TEAD transcriptional complex) in an individual in need thereof, comprising administering to the individual a compound of formula (I), formula (I′), formula (Ia), formula (Ia′), formula (Ib), formula (Ic), formula (Ib-1), or formula (Ib-2) or a pharmaceutically acceptable salt thereof (e.g., a therapeutically effective amount of a compound of formula (I), formula (I′), formula (Ia), formula (Ia′), formula (Ib), formula (Ic), formula (Ib-1), or formula (Ib-2) or a pharmaceutically acceptable salt thereof).

TEA domain transcription factors (TEADs) are downstream effectors of the Hippo signaling pathway. Currently, four isoforms of TEAD have been identified-TEAD1, TEAD2, TEAD3, and TEAD4. The TEAD isoforms share highly similar structures. The N-terminus of the four isoforms share a highly conserved 68-amino acid TEA/ATTS DNA-binding domain, which binds to the MCAT element (50-CATTCCA/T-30). The C-terminus contains the transactivation domain which recruits transcriptional coactivators YAP/TAZ.

The expression of TEAD proteins is up-regulated in many cancer types including gastric, colorectal, breast, and prostate cancers. TEAD hyperactivation plays a role in tumor progression, metastasis, cancer metabolism, immunity, and drug resistance and is correlated with poor survival in patients.

Selective binding of TEAD1 and/or TEAD4 is beneficial for optimizing anti-tumor efficacy while minimizing undesired effects. Inhibition of TEAD3 has been associated with off-target toxicity (e.g., kidney toxicity), while inhibition of TEAD2 may be pro-proliferative.

The TEAD isoform selectivity demonstrated by compounds disclosed herein is an advantage over TEAD inhibitors known in the art. For example, as demonstrated by Tang and Post using thermal shift assays, TEAD inhibitor VT3989 primarily interacts with TEAD1-3. The reported thermal shift data are provided in Table 2 below (Tang and Post, “The TEAD autopalmitoylation inhibitor VT3989 improves efficacy and increases durability of efficacy of osimertinib in preclinical EGFR mutant tumor models,” Proceedings of the American Association for Cancer Research Annual Meeting 2022; 2022 Apr. 8-13. Philadelphia (PA): AACR; Poster #5364). The data provided in Table 2 shows that VT3989 provides minimal selectivity over TEAD2 and 3, TEAD isoforms for which inhibition is undesirable. Furthermore, VT3989 shows lower levels of binding to TEAD4, a TEAD isoform for which inhibition is desirable. Each change in ΔTm (° C.) of 2.5° C. may be associated with an approximately 10-fold difference in binding affinity (Bhayani et al.; “Determination of dissociation constants of protein ligands by thermal shift assay,” Biochemical and Biophysical Research Communications, 2022, 560:1-6.)

TABLE 2
Thermal shift assay of TEAD isoforms 1-4 treated with TEAD
inhibitor VT3989 compared to inactive analog control.

| Isoform | VT3989 | Inactive Analog |
|---|---|---|
| TEAD1 ΔTm (° C.) | 11.6 | 0.2 |
| TEAD2 ΔTm (° C.) | 8.8 | 0.5 |
| TEAD3 ΔTm (° C.) | 8.5 | 0.2 |
| TEAD4 ΔTm (° C.) | 4.6 | 0.2 |

In some embodiments, the compounds disclosed herein selectively bind one or more TEAD isoforms. In certain embodiments, the compounds disclosed herein selectively bind TEAD1. In certain embodiments, the compounds disclosed herein selectively bind TEAD4. In certain embodiments, the compounds disclosed herein selectively bind TEAD1 and TEAD4. In some embodiments, the binding selectivity for TEAD1 is 10-fold, 100-fold, 1,000-fold, or 10,000-fold over TEAD2 and/or TEAD3. In some embodiments, the binding selectivity for TEAD4 is 10-fold, 100-fold, 1,000-fold, or 10,000-fold over TEAD2 and/or TEAD3. Evidence of the TEAD isoform selectivity demonstrated by compounds disclosed herein is provided in Example 174 below.

In some embodiments, the compounds disclosed herein are useful as a medical therapy for treating a disease, disorder or condition mediated by hyperactivation of YAP/TAZ-TEAD (e.g., TEAD1 and/or TEAD4) transcriptional co-activator complex. In some embodiments, the compounds disclosed herein are useful as a medical therapy for treating a disease, disorder or condition characterized by the hyperactivation of TEAD (e.g., TEAD1, TEAD2, TEAD3, and TEAD4 isoforms). In some embodiments, the disease, disorder or condition is characterized by the overexpression or genomic fusion or amplification of TEAD. TEAD1 and TEAD4 experience recurrent onco-fusions; for example, TEAD1-PARVA. These fusions drive higher expression of TEADs and may result in enhanced transcription of TEAD-target genes. Amplifications (genomic copy number gains) of TEAD4 occur in diverse cancers as part of the 12p13 locus in, e.g., ovarian and uterine carcinoma as well as testicular germ cell tumors. These genomic amplifications of TEAD4 are associated with strong increase in its mRNA expression and may drive high TEAD-YAP/TAZ transcriptional activity.

In certain embodiments, the TEAD isoform is TEAD1. In certain embodiments, the TEAD isoform is TEAD4. In some embodiments, the disease, disorder or condition is a cancer characterized by abnormal TEAD transcriptional complex activity (e.g., hyperactivation). Such cancers include but are not limited to breast cancer, lung cancer, gastric cancer, colorectal cancer, pancreatic cancer including pancreatic adenocarcinoma, mesothelioma including malignant mesothelioma, hepatocellular cancer, prostate cancer, head and neck cancer, renal cell carcinoma, and medulloblastomas. In some embodiments, the cancer is selected from pancreatic adenocarcinoma, hepatocellular cancer, breast cancer, and malignant mesothelioma. In certain embodiments, the cancer is malignant mesothelioma.

In some embodiments, the cancer is a breast cancer. In some embodiments, the cancer is a lung cancer. In some embodiments, the cancer is a gastric cancer. In some embodiments, the cancer is a colorectal cancer. In some embodiments, the cancer is a prostate cancer. In some embodiments, the cancer is a head and neck cancer. In some embodiments, the cancer is a renal cell carcinoma. In some embodiments, the cancer is a medulloblastoma. In some embodiments, the cancer is a metastatic cancer. In some embodiments, the cancer is medulloblastoma. In some embodiments, the cancer is a metastatic cancer. In some embodiments, the cancer is a metastatic breast cancer. In some embodiments, the cancer is a metastatic lung cancer. In some embodiments, the cancer is a metastatic gastric cancer. In some embodiments, the cancer is a metastatic colorectal cancer. In some embodiments, the cancer is a metastatic prostate cancer. In some embodiments, the cancer is a metastatic head and neck cancer. In some embodiments, the cancer is a metastatic renal cell carcinoma. In some embodiments, the cancer is a metastatic mesothelioma. In some embodiments, the cancer is a metastatic pancreatic cancer. In some embodiments, the cancer is a metastatic hepatocellular cancer.

In some embodiments, the disclosure provides for compounds of formula (I), formula (I′), formula (Ia), formula (Ia′), formula (Ib), formula (Ic), formula (Ib-1), or formula (Ib-2) for modulating TEAD activity. In some embodiments, the disclosure provides for pharmaceutically acceptable salts of compounds of formula (I), formula (I′), formula (Ia), formula (Ia′), formula (Ib), formula (Ic), formula (Ib-1), or formula (Ib-2) for modulating TEAD activity.

In some embodiments, the disclosure provides for compounds of formula (I), formula (I′), formula (Ia), formula (Ia′), formula (Ib), formula (Ic), formula (Ib-1), or formula (Ib-2) or a pharmaceutically acceptable salts thereof for use in medical therapy.

### EXAMPLES

The present disclosure will be further illustrated in the following Examples which are given for illustration purposes only and are not intended to limit the disclosure in any way.

### Abbreviations

Boc for tert-butyloxycarbonyl
DAST for diethylaminosulfur trifluoride
DBU for 1,8-diazabicyclo(5.4.0)undec-7-ene
DCC for dicyclohexylcarbodiimide
DCE for 1,1-dichloroethane
DCM for dichloromethane
DEA for diethanolamine
DEAD for diethyl azodicarboxylate
DIAD for diisopropyl azodicarboxylate
DIBAL for Diisobutylaluminium hydride
DIPEA for N,N-diisopropylethylamine, Hünig's base
DMA for N,N-dimethylacetamide
DMAP for 4-(dimethylamino)pyridine
DMF for N,N-dimethylformamide
DMSO for dimethylsulfoxide.
EDC for 1-Ethyl-3-(3-dimethylaminopropyl) carbodiimide
EtOAc for ethyl acetate
h for hours
HATU for N-[(dimethylamino)-1H-1,2,3-triazolo-[4,5-b]pyridin-1-ylmethylene]-N-methylmethanaminium hexafluorophosphate N-oxide
HBTU for (1H-benzotriazol-1-yloxy) (dimethylamino)-N,N-dimethylmethaniminium hexafluorophosphate
HOBT for N-hydroxybenzotriazole
HPLC for High Pressure Liquid Chromatography.
LAH for lithium aluminium hydride
IPA for isopropyl alcohol
LCMS for Liquid Chromatography-Mass Spectrometry
LDA for Lithium diisopropylamide
LiHMDS for Lithium bis(trimethylsilyl)amide
mCPBA for meta-chloroperoxybenzoic acid
MI for Molecular Ion
Min for minutes
MW for microwave
NBS for N-bromosuccinamide
NCS for N-chlorosuccinamide
NFOBS for N-fluoro-o-benzenedisulfonimide
NFSI for N-fluorobenzenesulfonimide
NHS for N-hydroxysuccinimide
NIS for N-iodosuccinamide
NMM for N-methylmorpholine
NMP for 1-methyl-2-pyrrolidinone
NMR for Nuclear Magnetic Resonance.
PdCl₂(PPh₃)₂ for Bis(triphenylphosphine)palladium chloride
Pd(dppf)₂Cl₂ for [1,1′-bis(diphenylphosphino)ferrocene]dichloropalladium (II)
Pd(dppf)₂Cl₂·DCM for [1,1′-bis(diphenylphosphino)ferrocene]dichloropalladium (II) complex with DCM
(Pd(dba)₂) for bis(dibenzylideneacetone)palladium
Rbf for round bottomed flask
RT for Retention Time.
SCX-2 for a silica-based sorbent with a chemically bonded propylsulfonic acid functional group
SFC for supercritical fluid chromatography
TBAF for tetra-n-butylammonium fluoride
TBDMS for tert-butyldimethylsilyl
TFAA for trifluoroacetic anhydride
TFA for trifluoroacetic acid
THF for tetrahydrofuran
TPP for tripotassium phosphate
Ts for toluenesulfonyl
XPhos-Pd-G1 for 2-Dicyclohexylphosphino-2′,4′,6′-triisopropyl-1,1′-biphenyl)[2-(2-aminoethyl)phenyl)]palladium (II) chloride
XPhos-Pd-G2 for Chloro(2-dicyclohexylphosphino-2′,4′,6′-triisopropyl-1,1′-biphenyl)[2-(2′-amino-1,1′-biphenyl)]palladium (II)

### Analytical Methods

Commercially available starting materials, reagents and dry solvents were used as supplied. Flash column chromatography or glass column chromatography was performed using Merck silica gel 230-400 mesh size. Flash chromatography was also performed on combi-flash RF Teledyne Isco machine. Preparative TLC was performed on Merck plates.

### Liquid Chromatography-Mass Spectrometry Methods

### Method-A

Method Name: -UC02_FAR1, Machine Details: -Water Acquity UPLC-H Class equipped with PDA and Acquity SQ detector, Column: Waters X-bridge C18, 50*2.1 mm, 2.5 micron, Column temperature: 35° C., Auto sampler temperature: 15° C., Mobile Phase A: 0.1% Formic acid in Milli Q water (PH=2.70), Mobile Phase B: 0.1% Formic acid in Milli Q water:Acetonitrile (10:90); Mobile phase gradient details: T=0 min (97% A, 3% B) flow: 0.8 mL/min; T=0.75 min (97% A, 3% B) flow: 0.8 mL/min; gradient to T=2.7 min (2% A, 98% B) flow: 0.8 mL/min; gradient to T=3 min (0% A, 100% B) flow: 1 mL/min; T=3.5 min (0% A, 100% B) flow: 1 mL/min; gradient to T=3.51 min (97% A, 3% B) flow: 0.8 mL/min; end of run at T=4 min (97% A, 3% B), Flow rate: 0.8 mL/min, Flow rate: −0.8 mL/min, Run Time: −4 min, UV Detection Method: -PDA, Wavelength: −200-500 nm; Mass parameter: Probe: -ESI, Mode of Ionisation: -positive and negative, Cone voltage: −30V and 10 V, capillary voltage: −3.0 KV, Extractor Voltage: −1 V, Rf Lens: −0.1 V, Temperature of source: −120° C., Temperature of Desolvation: −400° C. Cone Gas Flow: −100 L/hour, Desolvation Gas flow: −800 L/hour.

### Method-B

Method Name: -UC03_ABR2, Machine Details: -Waters Acquity Ultraperfomance LC connected with PDA and equipped with SQ detector, Column: Waters X-bridge C18, 50*4.6 mm, 3.5 micron, Column temperature: 35° C., Auto sampler temperature: 15° C., Mobile Phase A: 5 mM Ammonium Bicarbonate (pH=8.00) in Milli Q water, Mobile Phase B: Acetonitrile; Mobile phase gradient details: T=0 min (97% A, 3% B) Flow rate=1.0 ml/min, T=0.20 min (97% A, 3% B) Flow rate=1.0 ml/min; gradient to T=2.70 min (20% A, 80% B) Flow rate=1.0 ml/min; gradient to T=3.0 min (0% A, 100% B) Flow rate=1.2 ml/min; T=3.50 min (0% A, 100% B) Flow rate=1.2 ml/min; T=3.51 min (97% A, 3% B) Flow rate=1.0 ml/min; end of run at T=4.0 min (97% A, 3% B) Flow rate=1.0 ml/min, Run Time: −4 min, UV Detection Method: -PDA, Wavelength: −195 nm-500 nm; Mass parameter: Probe: -ESI, Mode of Ionisation: -Positive and Negative, Cone voltage: −30 and 10 V, capillary voltage: −3.0 KV, Extractor Voltage: −2 V, Rf Lens: −0.1 V, Temperature of source: −120° C., Temperature of Probe: −400° C., Cone Gas Flow: −100 L/Hr, Desolvation Gas flow: −800 L/Hr.

### High-Performance Liquid Chromatography Methods

### Method-A

Method Name: -HP04_BR1 Machine Details: -Water alliance e2695 with 2998 PDA detector, Column temperature: 25° C., Auto sampler temperature: 25° C., Mobile Phase A: 0.1% ammonium hydroxide solution in HPLC water Mobile Phase B: 100% ACETONITRILE; Mobile phase gradient details: T=0 min (10% A, 90% B) flow: 1 mL/min; T=7 min (90% A, 10% B) flow: 1 mL/min; gradient to T=9 min (100% A, 0% B) flow: 1 mL/min; gradient to T=14 min (100% A, 0% B) flow: 1 mL/min; T=14.01 min (10% A, 90% B) flow: 1 mL/min; gradient to T=17 min (10% A, 90% B) flow: 1 mL/min; end of run at T=17 min (10% A, 90% B), Flow rate: 1 mL/min, Run Time: −17 min, UV Detection Method: -PDA.

### Method-B

Method Name: -HP05_TFAR1.Machine Details: AGILENT TECHNOLOGY 1260 infinity series with PDA detector, Column temperature: 25° C., Auto sampler temperature: 25° C., Mobile Phase A: 0.05% Trifluoroacetic acid in HPLC water Mobile Phase B: 100% Acetonitrile; Mobile phase gradient details T=0 min (90% A, 10% B) flow: 1 mL/min; T=7 min (10% A, 90% B) flow: 1 mL/min; gradient to T=9 min (0% A, 100% B) flow: 1 mL/min; gradient to T=14 min (0% A, 100% B) flow: 1 mL/min; T=14.01 min (90% A, 10% B) flow: 1 mL/min; gradient to T=17 min (90% A, 10% B) flow: 1 mL/min; end of run at T=17 min (90% A, 10% B), Flow rate: 1 mL/min, Run Time: −17 min, UV Detection Method: -PDA.

### Method-C

Method Name: -HP06_TFAR1 Machine Details: -AGILENT TECHNOLOGY 1100series with PDA detector, Column temperature: 25° C., Auto sampler temperature: 25° C., Mobile Phase A: 0.05% Trifluoroacetic acid in HPLC water Mobile Phase B: 100% Acetonitrile; Mobile phase gradient details T=0 min (90% A, 10% B) flow: 1 mL/min; T=7 min (10% A, 90% B) flow: 1 mL/min; gradient to T=9 min (0% A, 100% B) flow: 1 mL/min; gradient to T=14 min (0% A, 100% B) flow: 1 mL/min; T=14.01 min (90% A, 10% B) flow: 1 mL/min; gradient to T=17 min (90% A, 10% B) flow: 1 mL/min; end of run at T=17 min (90% A, 10% B), Flow rate: 1 mL/min, Run Time: −17 min, UV Detection Method: -PDA.

### NMR

¹H Nuclear magnetic resonance (NMR) spectroscopy was carried out using a Bruker Avance-400 instrument operating at 400 MHz using the stated solvent at room temperature unless otherwise stated. Samples were prepared as solutions in a suitable deuterated solvent and referenced to the appropriate internal non-deuterated solvent peak or tetramethylsilane. Chemical shifts were recorded in ppm (δ) downfield of tetramethylsilane. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; dt, doublet of triplets; m, multiplet; br, broad.

### Purification Methods

### Preparative Purification by Reverse Phase HPLC

### Preparatory HPLC Method-A

Biotage-FC-01 with binary pump with UV/Visible wave-length detector, Column: YMC, 120 g, 50 μm, column temperature: room temp., mobile phase A: 0.1% formic acid in water, mobile phase B: Acetonitrile: mobile phase gradient details: t=0.01 min (100% A, 0% B); t=3 min (85% A, 15% B); gradient to t=25 min (55% A, 45% B); t=35 min (0% A, 100% B); gradient to end of run at t=45 min (100% A, 0% B); flow rate: 80 mL/min, analysis time 45 min.

### Synthesis

Several methods for the chemical synthesis of compounds of the present application are described herein. These and/or other well-known methods may be modified and/or adapted in various ways to facilitate the synthesis of additional compounds within the scope of the present application and claims. Such alternative Methods and modifications should be understood as being within the spirit and scope of this application and claims. Accordingly, Methods set forth in the following descriptions, Schemes and Examples are intended for illustrative purposes and are not to be construed as limiting the scope of the disclosure.

In one approach (Scheme 1), compounds of formula [IIb] may be prepared by the reaction of a substituted Het-aromatic carboxylic acid of formula [II] with amide coupling reagent like HATU in DMF or DCM as solvent, with an amine of general formula [III] in the presence of a tertiary amine base such as DIPEA. The reaction is suitably conducted at RT. After reaction work up, typically by liquid-liquid extraction, the reaction product is purified by flash column chromatography, reverse phase preparative HPLC or re-crystallisation (Method A).

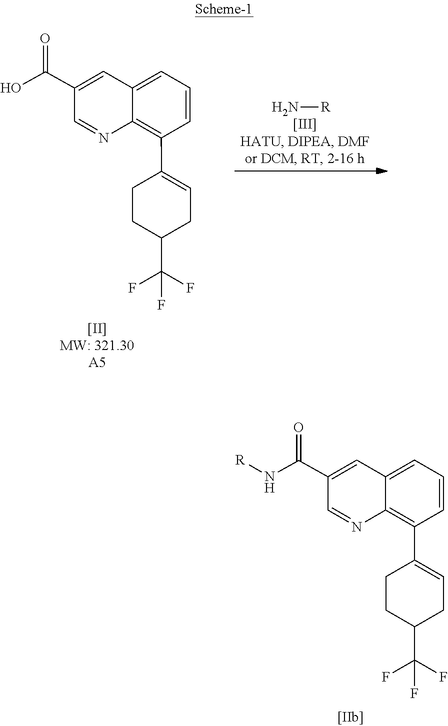

The following compounds were prepared according to the Methods described above using the indicated intermediates.

### Example 1—Synthesis of N-isopropyl-8-(4-(trifluoromethyl)cyclohex-1-en-1-yl)quinoline-3-carboxamide (Compound 1)

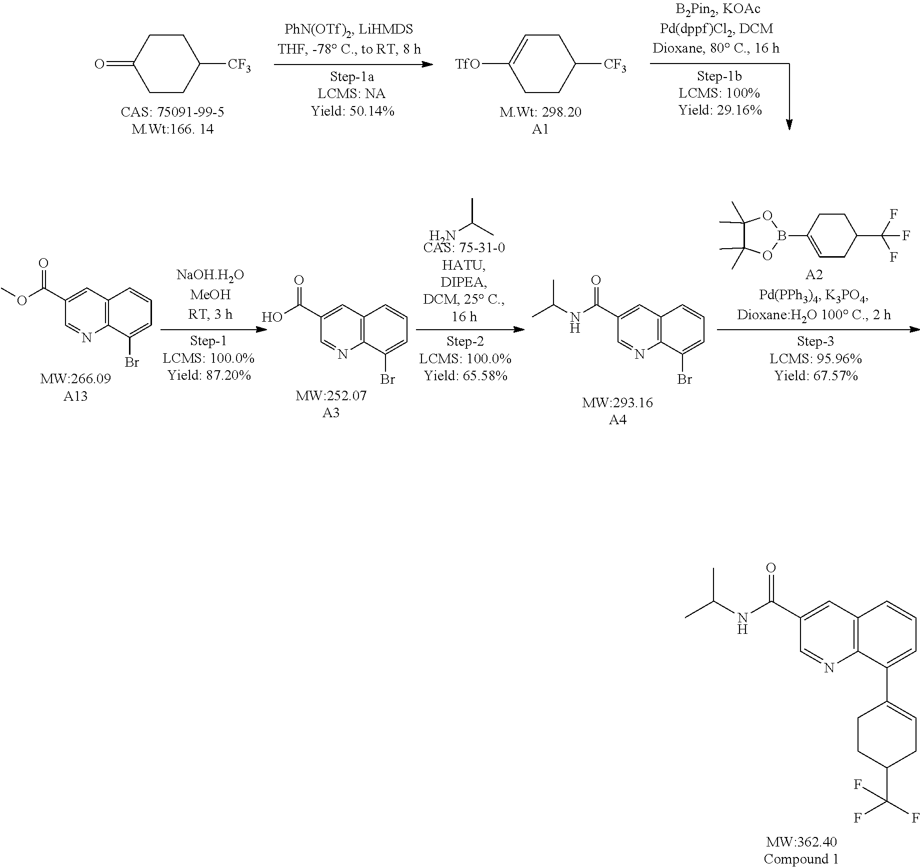

### Step-1a:

In 250 mL three neck RB flask, 4-(trifluoromethyl)cyclohexan-1-one CAS No: 75091-99-5, (0.5 g, 3.01 mmol, 1.0 eq.), in THF (31.5 mL) was added dropwise lithium bis(trimethylsilyl)amide (3.6 mL, 3.61 mmol, 1M solution in THF) over a period of 30 min with stirring at −78° C. then allowed stirring continue for another 1 h at −78° C., after 1 h solution of N-phenyltrifluoromethanesulfonimide (1.0 g, 3.01 mmol, 1.0 eq) in THF (4 mL) was added dropwise with stirring over a period of 30 min. Then the reaction mixture was stirred at −78° C. for another 2 h and slowly warmed to room temperature with stirring over a period of 6 h. The reaction was monitored on TLC (using EA:Hexane; 0.1:9.9 as mobile phase) which confirmed that the reaction got completed after 6 h of stirring at room temperature. The reaction mixture was extracted with EtOAc (3×10 mL). combined organic extracts were washed with water, dried over Na₂SO₄, and filtered. The filtrate was evaporated and the residue was purified by silica gel column chromatography (10% EtOAc in Hexane) yielded 4-(trifluoromethyl)Cyclohex-1-en-1-yl trifluoromethanesulfonate as a white solid (A1, 0.45 g, 1.509 mmol, Yield: 50.14%).

¹H NMR (CDCl₃, 400 MHz): δ ppm 5.80-5.81 (m, 1H), 2.51-2.47 (m, 2H), 2.43-2.30 (m, 2H), 2.20-2.15 (m, 1H), 1.84-1.73 (m, 1H), 0.83-0.90 (m, 1H). Note: aliphatic impurities with trace amount of PhN(OTf)₂.

### Step-1b:

In 250 mL three neck RB flask, To a solution of 4-(trifluoromethyl)-cyclohex-1-enyl trifluoromethanesulfonate CAS: 73183-34-3, (A1, 0.1 g, 0.335 mmol, 1.0 eq) in dioxane (3.8 mL) was added bis(pinacolato)diboron, (0.102 g, 0.402 mmol, 1.2 eq), potassium acetate (0.109 g, 1.11 mmol, 3.0 eq), PdCl₂ (dppf) (0.074 g, 0.100 mmol, 0.3 eq), under a nitrogen atmosphere. The reaction mixture was heated at 80° C. with stirring for 16 h, The reaction was monitored on TLC (using EA:Hexane; 1:9 as mobile phase) which confirmed that the reaction got completed after 16 h of stirring at 80° C. and then cooled to room temperature, and filtered through celite bed. The filtrate was evaporated in vacuum and the residue was purified by silica gel column chromatography (10% EtOAc/hexane) yielded 4,4,5,5-tetramethyl-2-(4-(trifluoromethyl)cyclohex-1-en-1-yl)-1,3,2-dioxaborolane (A2, 0.027 g, 0.097 mmol, Yield: 29.16%).

¹H NMR (DMSO-d6, 400 MHz): δ ppm 6.54-6.53 (m, 1H), 2.38-2.31 (m, 2H), 2.27-2.25 (m, 1H), 2.21-2.20 (m, 2H), 2.03-1.99 (m, 1H) 1.49-1.47 (m, 1H), 1.30 (s, 12H). Note: Minor aliphatic impurities observed

### Step-1:

A solution of Methyl 8-bromoquinoline-3-carboxylate, (A13, 5.0 g, 18.791 mmol, 1.0 eq.) and NaOH (1.5 g, 37.581 mmol, 2.0 eq.) in methanol and water (50 mL, 9:1) was stirred for 2 h at room temperature. The reaction mixture was acidified with 1N HCl (40 mL; pH=3) and allowed to stir for 30 minutes at 0° C. to afford pale yellowish solid. The reaction was monitored by TLC (using EA:Hex; 3:7 as mobile phase) which confirmed the completion after 2 h of stirring at room temperature. The resulting solid was filtered with high vacuum and yielded 8-bromoquinoline-3-carboxylic acid (A3, 4.13 g, 16.38 mmol, Yield: 87.20%).

¹H NMR (DMSO-d6, 400 MHz): δ ppm 7.64 (t, J=8.0 Hz, 1H), 8.25-8.31 (m, 2H), 9.05 (d, J=2.4 Hz, 1H), 9.40 (d, J=2.4 Hz, 1H)

LCMS (Method A): 1.736 min, 100.0%, MS: ES+252.00 [M]⁺

### Step-2:

In 250 mL three neck RB flask, 8-bromoquinoline-3-carboxylic acid (A3, 4.0 g, 15.873 mmol, 1.0 eq.), Isopropyl amine CAS No: 75-31-0 (1.19 g, 15.873 mmol, 1.0 eq.), and DIPEA (6.2 g, 47.62 mmol, 3.0 eq.) were stirred with HATU (6.59 g, 17.46 mmol, 1.1 eq.) in DMF (40 mL, 10 v) for 16 h at room temperature. The reaction was monitored on TLC (using EA:Hexane; 1:1 as mobile phase) which confirmed that the reaction got completed after 16 h of stirring at room temperature. The resulting reaction mixture was diluted with ice-cold water (200 mL) and allowed to stirred for 1 h. The yellowish solid obtained was filtered through buchner funnel to yield 8-Bromo-N-isopropylquinoline-3-carboxamide (A4, 3.05 g, 10.41 mmol, Yield: 65.58%).

¹H NMR (DMSO-d6, 400 MHz): δ ppm 9.34 (d, J=2.0 Hz, 1H), 8.87 (d, J=2.4 Hz, 1H), 8.67 (d, J=8.8 Hz, 1H), 8.26-8.24, (m, 1H), 8.16-8.13 (m, 1H), 7.62 (d, J=8.0 Hz, 1H), 4.22-4.13 (m, 1H), 1.23 (d, J=6.8 Hz, 6H),

LCMS (Method A): 1.852 min, 100.0%, MS: ES+293.01 [M+H]

### Step-3:

To a stirred solution of 8-bromo-N-isopropylquinoline-3-carboxamide (A4, 1.2 g, 4.093 mmol, 1.0 eq.), and 2-(4,4-difluorocyclohex-1-en-1-yl)-4,4,5,5-tetramethyl-1,3,2-dioxaborolane (A2, 1.24 g, 4.503 mmol, 1.1 eq.), in 1,4-dioxane and water (14.5 mL, 9:1), was added tripotassium phosphate (1.21 g, 12.28 mmol, 3.0 eq.) and purged with nitrogen for 30 minutes, followed by addition of Pd(dppf)Cl₂ (0.94 g, 0.819 mmol, 0.2 eq.), the resulting reaction mixture heated up to 100° C. for 16 h at room temperature. The reaction was monitored on TLC (using EA:Hexane; 3:7 as mobile phase) which confirmed that the reaction got completed after 16 h. The resulting reaction mixture was filtered through celite bed and the filtrate was concentrated under reduced pressure. The obtained crude residue (2.5 g) was purified by silica gel (60-120 mesh) as stationary phase (25% EtOAc in hexane) yielded N-isopropyl-8-(4-(trifluoromethyl)cyclohex-1-en-1-yl)quinoline-3-carboxamide (Compound 1, 1.0 g, 2.759 mmol, Yield: 67.57%).

¹H NMR (DMSO-d6, 400 MHz): δ ppm 9.24 (d, J=2.4 Hz, 1H), 8.78 (d, J=2.4 Hz, 1H), 8.58 (d, J=7.6 Hz, 1H), 8.01-7.98 (m, 1H), 7.63-7.62 (m, 2H), 5.87 (br s, 1H), 4.18-4.13 (m, 1H), 2.80-2.68 (m, 3H), 2.31-2.27 (m, 1H), 2.09-2.06 (m, 1H), 1.73-1.67 (m, 1H), 1.21 (d, J=8.0 Hz, 6H). Note: CF3-CH proton merged with DMSO solvent peak which is clearly observed in MeOD NMR.

¹H NMR (MeOD, 400 MHz): δ ppm 9.24 (d, J=2.4 Hz, 1H), 8.74 (d, J=2.4 Hz, 1H), 7.96 (dd, J=7.6, 2.0 Hz, 1H), 7.68-7.62 (m, 2H), 5.87 (br s, 1H), 4.33-4.26 (m, 1H), 2.79-2.64 (m, 3H), 2.55-2.49 (m, 1H), 2.41-2.36 (m, 1H), 2.18-2.14 (m, 1H), 1.92-1.83 (m, 1H), 1.33 (d, J=6.8 Hz, 6H). Note: —CONH Proton exchanged in MeOD.

LCMS (Method A): 2.239 min, 96.73%, MS: ES+363.10 [M+H]

HPLC (Method B): 8.327 min, 97.65%, @254 nm.

### Example 2—Synthesis of N—((S)-1-hydroxypropan-2-yl)-8-(4-(trifluoromethyl)cyclohex-1-en-1-yl)quinoline-3-carboxamide (Compound 2)

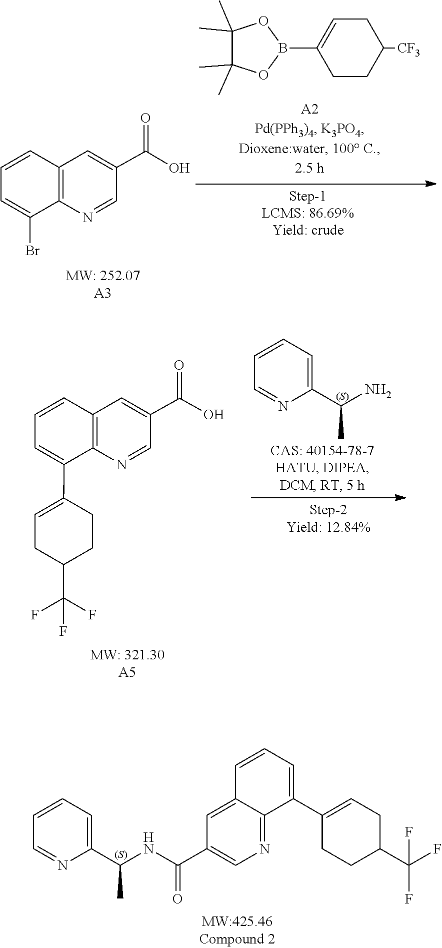

### Step-1:

To a stirred solution of 8-bromoquinoline-3-carboxylic acid (A3, 0.3 g, 1.190 mmol, 1.0 eq), 4,4,5,5-tetramethyl-2-(4-(trifluoromethyl)cyclohex-1-en-1-yl)-1,3,2-dioxaborolane (A2, 0.328 g, 1.190 mmol, 1.0 eq) and K₃PO₄ (1.2 g, 5.952 mmol, 5.0 eq) in 1,4 Dioxane:water (4 mL, 9:1) was added Pd(PPh₃)₄ (0.137 g, 0.119 mmol, 0.1 eq) under N₂. The resulting mixture was stirred at 100° C. for 2.5 h. The reaction was monitored on TLC (using EA:Hex; 7.0:3.0 as mobile phase) which confirmed that the reaction got completed after 2.5 h of stirring at 100° C. The resulting reaction mixture was diluted with water (20 mL) and extracted with EtOAc (3×50 mL). The combined organic layers were dried over Na₂SO₄ and concentrated under reduced pressure to afford 0.3 g (crude) 8-(4-(trifluoromethyl)cyclohex-1-en-1-yl)quinoline-3-carboxylic acid (A5, 0.3 g, 0.934 mmol, Yield: Crude). Note: The crude compound used as such for next step without further purification.

LCMS (Method A): 2.464 min, 86.69%, 254.0 nm, MS: ES+322.11 (M+1)

### Step-2:

A solution of 8-(4-(trifluoromethyl)cyclohex-1-en-1-yl)quinoline-3-carboxylic acid (A5, 0.3 g, 0.9345 mmol, 1.0 eq), HATU (1.0 g, 2.803 mmol, 3.0 eq) and DIPEA (0.180 g, 1.401 mmol, 1.5 eq) in DCM (3 mL) at 0° C. under N₂ atmosphere stirred for 20 min, then added CAS: 40154-78-7 (0.2 g, 1.027 mmol, 1.1 eq) under Nitrogen. The resulting mixture was stirred for 5 h at room temperature. The reaction was monitored on TLC (using EA:Hex; 7.0:3.0 as mobile phase) which confirmed that the reaction got completed after 5 h. The resulting reaction mixture was quenched with ice-cold water (10 mL) and extracted with EtOAc (3×25 mL). The combined organic layers were dried over Na₂SO₄ and concentrated under reduced pressure to afford 0.5 g crude. The obtained crude material was purified by flash column chromatography using silica gel (230-400 mess) as stationary phase (45% EtOAc in hexane) yielding N—((S)-1-(pyridin-2-yl)ethyl)-8-(4-(trifluoromethyl)cyclohex-1-en-1-yl)quinoline-3-carboxamide (Compound 2, 0.065 g, 0.152 mmol, Yield: 12.84% (yield over 2 steps))

¹H NMR (DMSO-d6, 400 MHz): δ ppm, 9.29 (br s, 1H), 9.19 (d, J=7.6 Hz, 1H), 8.89 (br s, 1H), 8.54 (d, J=4.4 Hz, 1H), 8.02 (t, J=4.4 Hz, 1H), 7.78 (t, J=7.2 Hz, 1H), 7.65-7.62 (m, 2H), 7.48 (d, J=8.0 Hz, 1H), 7.28 (t, J=5.6 Hz, 1H), 5.87 (s, 1H), 5.28-5.23 (m, 1H), 2.81-2.69 (m, 3H), 2.46-2.42 (m, 1H), 2.32-2.29 (m, 1H), 2.1-2.07 (m, 1H), 1.71-1.67 (m, 1H), 1.55 (d, J=6.8 Hz, 3H).

¹H NMR (MeOD, 400 MHz): δ ppm, 9.27 (d, J=2.4 Hz, 1H), 8.85 (d, J=2.4 Hz, 1H), 8.56 (d, J=1.6 Hz, 1H), 7.9 (dd, J=1.6 Hz, 1H), 7.86 (td, J=7.6 Hz, 1H), 7.70 (m, 2H), 7.54 (d, J=8.0 Hz, 1H), 7.35 (t, J=4.8 Hz, 1H), 5.87 (br s, 1H), 5.35 (q, J=7.1 Hz, 1H), 2.83-2.71 (m, 1H), 2.68-2.64 (m, 2H), 2.56-2.50 (m, 1H), 2.41-2.37 (m, 1H), 2.18-2.15 (m, 1H), 1.81-1.70 (m, 1H), 1.66 (d, J=2.0 Hz, 3H).

LCMS (Method A): 2.295 min, 100%, 254.0 nm, MS: ES+426.23 (M+1)

HPLC (Method B): 6.213 min, 99.75%, 254.0 nm

Chiral HPLC: 2.28 min, 100%, 245.0 nm

### Example 3—Synthesis of N—((S)-1-(6-aminopyridin-2-yl)ethyl)-8-(4-(trifluoromethyl)cyclohex-1-en-1-yl)quinoline-3-carboxamide formate (Compound 3)

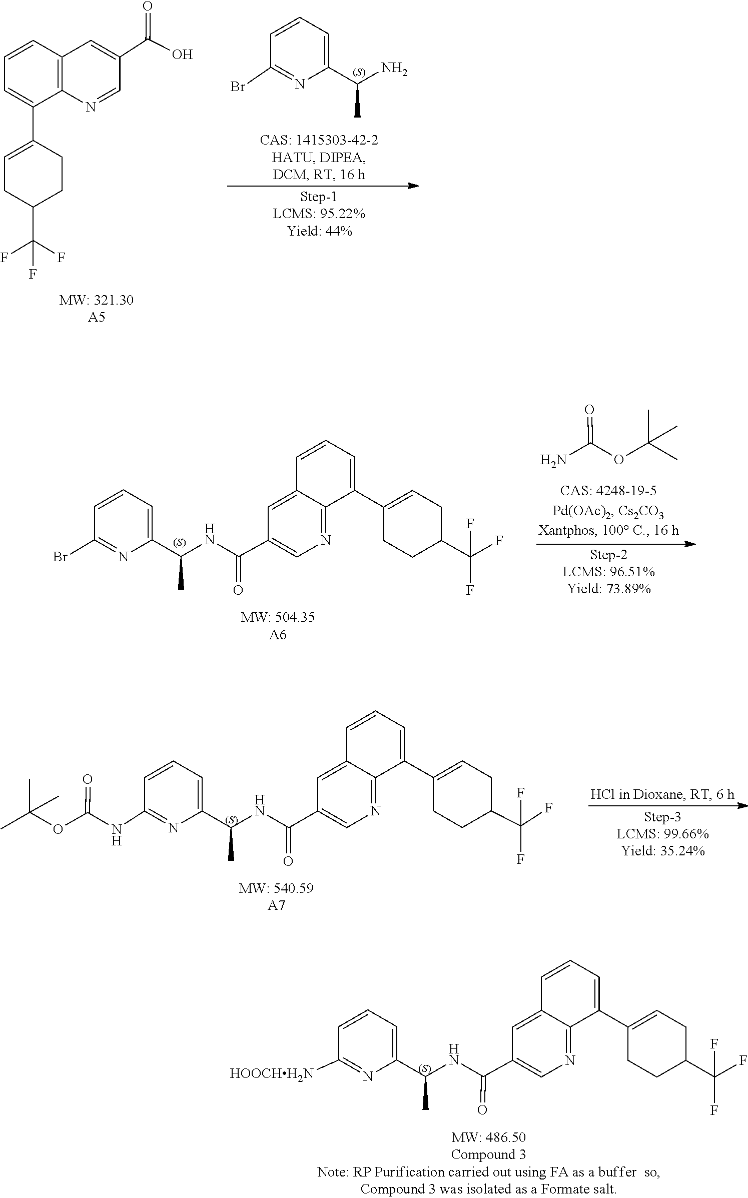

### Step-1:

A solution of 8-(4-(trifluoromethyl)cyclohex-1-en-1-yl)quinoline-3-carboxylic acid (A5, 0.3 g, 0.93 mmol, 1.0 eq), HATU (0.530 g, 1.39 mmol, 1.5 eq) and DIPEA (0.48 ml, 2.79 mmol, 3.0 eq) in DCM (5 mL) at 0° C. under nitrogen atmosphere stirred for 5 min, then CAS: 1415303-42-2 (0.265 g, 1.11 mmol, 1.2 eq), was added under nitrogen atmosphere. The resulting mixture was stirred for 16 h at room temperature. The reaction was monitored on TLC (using EA:Hex; 8:2 as mobile phase) which confirmed that the reaction got completed after 16 h. The resulting reaction mixture was quenched with water (10 mL) and extracted with DCM (3×25 mL). The combined organic layers were dried over Na₂SO₄ and concentrated under reduced pressure to afford 0.3 g crude. The obtained crude material was purified by flash column chromatography using silica gel (230-400 mesh) as stationary phase (50% Ethyl acetate in hexane as gradient) yielding N—((S)-1-(6-bromopyridin-2-yl)ethyl)-8-(4-(trifluoromethyl)cyclohex-1-en-1-yl)quinoline-3-carboxamide (A6, 0.21 g, 0.416 mmol, Yield: 44%).

¹H NMR (DMSO-d⁶, 400 MHz): δ 9.28-9.25 (m, 2H), 8.89 (d, J=2.4 Hz, 1H), 8.03-8.00 (m, 1H), 7.75 (t, J=7.6 Hz, 1H), 7.67-7.62 (m, 2H), 7.55-7.51 (m, 2H), 5.88 (br s, 1H), 5.22-5.15 (m 1H), 2.82-2.60 (m, 3H), 2.51-2.50 (m, 1H), 2.29-2.25 (m, 1H), 2.10-2.07 (m, 1H), 1.75-1.64 (m, 1H). 1.54 (d, J=7.2 Hz, 3H)

LCMS (Method A): 2.817 min, 95.22%, 254.0 nm, MS: ES+506.04 (M+2)

### Step-2:

A solution of N—((S)-1-(6-bromopyridin-2-yl)ethyl)-8-(4-(trifluoromethyl)cyclohex-1-en-1-yl)quinoline-3-carboxamide (A6, 0.19 g, 0.37 mmol, 1.0 eq), Pd(OAc)₂ (0.012 g, 0.037 mmol, 0.1 eq), xantphos (0.021 g, 0.037 mmol, 0.1 eq) and Cs₂CO₃ (0.360 g, 1.11 mmol, 3.0 eq) in Dioxane (10 mL) was prepared at RT. The resulting mixture was purge with nitrogen for 5 min, then added CAS: 4248-19-5 (0.051 g, 0.44 mmol, 1.2 eq) under Nitrogen atmosphere. The resulting mixture was stirred for 16 h at 100° C. The reaction was monitored by TLC (using EA:Hex; 70:30 as mobile phase) which confirmed that the reaction got completed after 16 h. The resulting reaction mixture was quenched with water (10 mL) and extracted with EtOAc (3×25 mL). The combined organic layers were dried over Na₂SO₄ and concentrated under reduced pressure to afford 0.19 g crude. The obtained crude material was purified by flash column chromatography using silica gel (230-400 mesh) as stationary phase desired product was eluted in 30% Ethyl acetate in hexane as gradient afforded tert-butyl (6-((1S)-1-(8-(4-(trifluoromethyl)cyclohex-1-en-1-yl)quinoline-3-carboxamido)ethyl) pyridin-2-yl) carbamate (A7, 0.15 g, 0.27 mmol, Yield: 73.89%).

¹H NMR (DMSO-d₆, 400 MHz): δ ppm, 9.75 (s, 1H), 9.33 (d, J=1.6 Hz, 1H), 9.07 (d, J=7.6 Hz, 1H), 8.89 (d, J=2.4 Hz, 1H), 8.05-8.03 (m, 1H), 7.74-7.64 (m, 4H), 7.12 (d, J=7.2 Hz, 1H), 5.87 (br s, 1H), 5.15-5.12 (m, 1H), 2.80-2.67 (m, 4H), 2.33-2.29 (m, 1H), 2.10-2.08 (m, 1H), 1.73-1.68 (m, 1H), 1.5 (d, J=7.2 Hz, 3H) 1.48 (s, 9H)

LCMS (Method A): 2.977 min, 96.51%, 254.0 nm, MS: ES+541.3 (M+1)

### Step-3:

To a solution of tert-butyl (6-((1S)-1-(8-(4-(trifluoromethyl)cyclohex-1-en-1-yl)quinoline-3-carboxamido)ethyl) pyridin-2-yl) carbamate (A7, 0.15 g, 0.277 mmol, 1.0 eq), in dioxane (10 mL) was added HCl in dioxane [4 M] (5 mL) at RT under nitrogen atmosphere. The resulting mixture was stirred for 16 h at room temperature. The reaction was monitored by TLC (using EA:Hex; 1:1 as mobile phase) which confirmed that the reaction got completed after 16 h. The resulting reaction mixture was concentrated under reduced pressure to afford 0.15 g crude as HCl salt. The crude material was purified by reverse phase prep HPLC purification (Method-A 0.1% formic acid in water acetonitrile) to obtained the desired N—((S)-1-(6-aminopyridin-2-yl)ethyl)-8-(4-(trifluoromethyl)cyclohex-1-en-1-yl)quinoline-3-carboxamide format (Compound 3, 0.043 g, 0.097 mmol, Yield: 35.24%).

¹H NMR (DMSO-d6, 400 MHz): δ ppm, 9.29 (br s, 1H), 9.13 (br s, 1H), 8.89 (br s, 1H), 8.04-8.01 (m, 1H), 7.66-7.63 (m, 2H), 7.42 (t, J=8.0 Hz, 1H), 6.60 (d, J=7.2 Hz, 1H), 6.41 (d, J=7.6 Hz, 1H), 5.87 (br s, 1H), 5.07-5.04 (br s, 1H), 2.80-2.67 (m, 3H), 2.51-2.50 (m, 1H), 2.29-2.25 (m, 1H), 2.10-2.07 (m, 1H), 1.72-1.64 (m, 1H), 1.51 (d, J=6.8 Hz, 3H) Note: —NH₂ protons aren't visible and compound in the form of format salt.

¹H NMR (MeOD, 400 MHz): δ ppm, 9.28 (d, J=2.4 Hz, 1H), 8.86 (d, J=2.4 Hz, 1H), 7.99 (dd, J=8.0, 1.6 Hz, 1H), 7.70-7.63 (m, 3H), 6.82 (d, J=7.2 Hz, 1H), 6.70 (d, J=8.4 Hz, 1H), 5.86 (br s, 1H), 5.21-5.15 (m, 1H 2.78-2.65 (m, 3H), 2.55-2.51 (m, 1H), 2.41-2.34 (m, 1H), 2.18-2.15 (m, 1H), 1.92-1.83 (m, 1H), 1.66 (d, J=7.2 Hz, 3H). Note: —NH and NH₂ protons aren't visible and compound in the form of format salt.

LCMS (Method B): 2.98 min, 99.60%, 254.0 nm, MS: ES+441.07 (M+1),

HPLC (Method A): 8.772 min, 99.60%, 254.0 nm

Chiral HPLC: 3.76 min, 100%, 240.0 nm

### Example 4—Synthesis of N—((S)-1-hydroxypropan-2-yl)-8-(4-(trifluoromethyl)cyclohex-1-en-1-yl)quinoline-3-carboxamide (Compound 4)

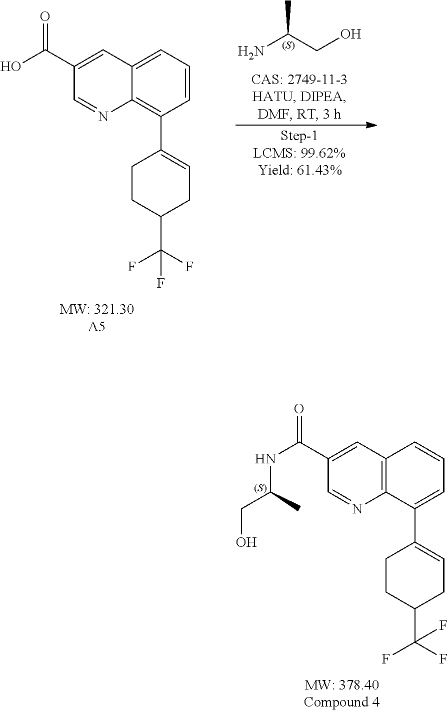

### Step-1:

To a solution of 8-(4-(trifluoromethyl)cyclohex-1-en-1-yl)quinoline-3-carboxylic acid (A5, 0.17 g, 0.529 mmol, 1.0 eq), HATU (0.30 g, 0.791 mmol, 1.5 eq) and DIPEA (0.20 g, 1.587 mmol, 3.0 eq) in DMF (1.7 mL) at 0° C. under nitrogen atmosphere stirred for 30 min, was added CAS: 2749 Nov. 3 (0.043 g, 0.582 mmol, 1.1 eq) under Nitrogen. The resulting mixture was stirred for 3 h at room temperature. The reaction was monitored by TLC (using 100% Ethyl acetate as mobile phase) which confirmed that the reaction got completed after 3 h. The resulting reaction mixture was quenched with ice-cold water (10 mL) and extracted with EtOAc (3×15 mL). The combined organic layers were dried over Na₂SO₄ and concentrated under reduced pressure to afford 0.22 g crude. The obtained crude material was purified by flash column chromatography using silica gel (230-400 mesh) as stationary phase (80% Ethyl acetate in hexane as gradient) yielding N—((S)-1-hydroxypropan-2-yl)-8-(4-(trifluoromethyl)cyclohex-1-en-1-yl)quinoline-3-carboxamide (Compound 4, 0.123 g, 0.325 mmol, Yield: 61.43%).

¹H NMR (DMSO-d6, 400 MHz): δ ppm, 9.25 (d, J=2.0 Hz, 1H), 8.80 (d, J=2.4 Hz, 1H), 8.47 (d, J=8.0 Hz, 1H), 8.01-7.99 (m, 1H), 7.64-7.63 (m, 2H), 5.87 (br s, 1H), 4.80 (t, J=6.0 Hz, 1H), 4.10-4.07 (m, 1H), 3.54-3.50 (m, 1H), 2.80-2.67 (m, 3H), 2.44-2.42 (m, 1H), 2.33-2.25 (m, 1H), 2.10-2.07 (m, 1H), 1.75-1.64 (m, 1H), 1.18 (d, J=6.8 Hz, 3H). Note: CF3-CH Proton merge with DMSO solvent peak which is clearly observed in MeOD NMR spectrum

¹H NMR (MeOD, 400 MHz): δ ppm, 9.25 (d, J=2.4 Hz, 1H), 8.78 (d, J=2.0 Hz, 1H), 7.96 (dd, J=7.6, 2.0 Hz, 1H), 7.69-7.62 (m, 2H), 5.87 (br s, 1H), 4.32-4.26 (m, 1H), 3.67 (d, J=5.6 Hz, 2H), 2.81-2.75 (m, 1H), 2.68-2.64 (m, 2H), 2.55-2.51 (m, 1H), 2.40-2.34 (m, 1H), 2.18-2.15 (m, 1H), 1.93-1.82 (m, 1H), 1.32 (d, J=6.8 Hz, 3H). Note: —OH and —NH protons might exchange in MeOD.

LCMS (Method A): 2.236 min, 99.62%, 254.0 nm, MS: ES+379.12 (M+1)

HPLC (Method A): 7.941 min, 99.26%, 254.0 nm

Chiral HPLC: 4.73 min, 50.10%, 240.0 nm; 5.10 min, 49.48%, 240.0 nm

### Example 5—Synthesis of N-(pyridin-2-ylmethyl)-8-(4-(trifluoromethyl)cyclohex-1-en-1-yl)quinoline-3-carboxamide (Compound 5)

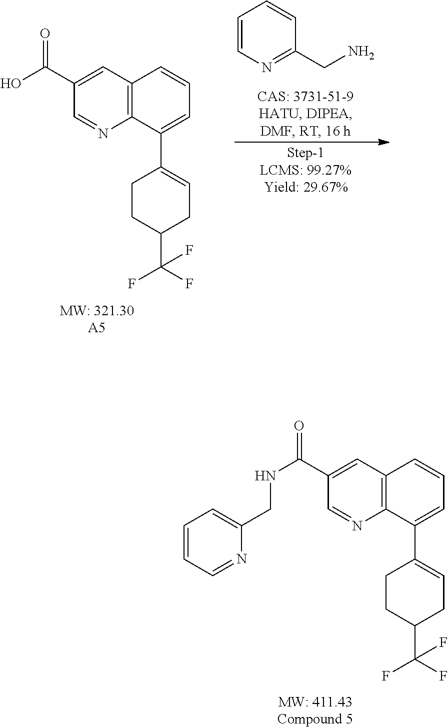 [SMILES: O=C(O)c1cnc2c(C3=CCC(C(F)(F)F)CC3)cccc2c1 ; NCc1ccccn1 ; O=C(NCc1ccccn1)c1cnc2c(C3=CCC(C(F)(F)F)CC3)cccc2c1]

### Step-1:

To a solution of 8-(4-(trifluoromethyl)cyclohex-1-en-1-yl)quinoline-3-carboxylic acid (A5, 0.15 g, 0.466 mmol, 1.0 eq), HATU (0.26 g, 0.700 mmol, 1.5 eq) and DIPEA (0.24 ml, 1.400 mmol, 3.0 eq) in DMF (1.5 mL) was stirred for 15 min at 0° C. under nitrogen atmosphere, was added CAS: 3731-51-9 (0.055 g, 0.513 mmol, 1.1 eq). The resulting mixture was stirred for 16 h at room temperature. The reaction was monitored by TLC (using 100% Ethyl acetate as mobile phase) which confirmed that the reaction got completed after 16 h. The resulting reaction mixture was quenched with water (10 mL) and extracted with EtOAc (3×15 mL). The combined organic layers were dried over Na₂SO₄ and concentrated under reduced pressure to afford 0.20 g crude. The obtained crude material was purified by flash column chromatography using silica (230-400 mesh) as stationary phase (70% Ethyl acetate in hexane as gradient) yielding N-(pyridin-2-ylmethyl)-8-(4-(trifluoromethyl)cyclohex-1-en-1-yl)quinoline-3-carboxamide (Compound 5, 0.057 g, 0.138 mmol, Yield: 29.67%).

¹H NMR (DMSO-d6, 400 MHz): δ ppm 9.46 (t, J=6 Hz, 1H) 9.31 (d, J=2.4 Hz, 1H), 8.89 (d, J=2.0 Hz, 1H) 8.53 (d, J=4.0 Hz, 1H), 8.04-8.00 (m, 1H), 7.79 (dt J=7.6, 1.6 Hz, 1H), 7.67-7.62 (m, 2H), 7.41 (d J=7.6 Hz, 1H), 7.29 (t, J=5.2 Hz, 1H), 5.88 (br s, 1H), 4.65 (d, J=6.0 Hz, 2H), 2.81-2.78 (m, 1H), 2.72-2.67 (m, 2H), 2.33-2.25 (m, 1H), 2.10-2.07 (m, 1H), 1.75-1.72 (m, 1H)

¹H NMR (MeOD, 400 MHz): 9.28 (d, J=2.0 Hz, 1H), 8.82 (d, J=2.4 Hz, 1H), 8.51 (d, J=5.2 Hz, 1H), 7.95 (dd J=8.0, 2.0 Hz 1H), 7.84 (td J=7.6, 1.6 Hz, 1H), 7.67-7.60 (m, 2H), 7.49 (d, J=8.0 Hz, 1H), 7.34-7.31 (m, 1H), 5.84 (br s, 1H), 4.76 (s, 2H), 2.80-2.73 (m, 1H), 2.67-2.62 (m, 2H), 2.54-2.48 (m, 1H), 2.38-2.30 (m, 1H), 2.15-2.12 (m, 1H), 1.87-1.82 (m, 1H)

LCMS (Method A): 2.145 min, 99.27%, 254.0 nm, MS: ES+412.17 (M+1)

HPLC (Method A): 8.707 min, 98.45%, 254.0 nm

### Example 6—Synthesis of N—((S)-1-methoxypropan-2-yl)-8-(4-(trifluoromethyl)cyclohex-1-en-1-yl)quinoline-3-carboxamide (Compound 6)

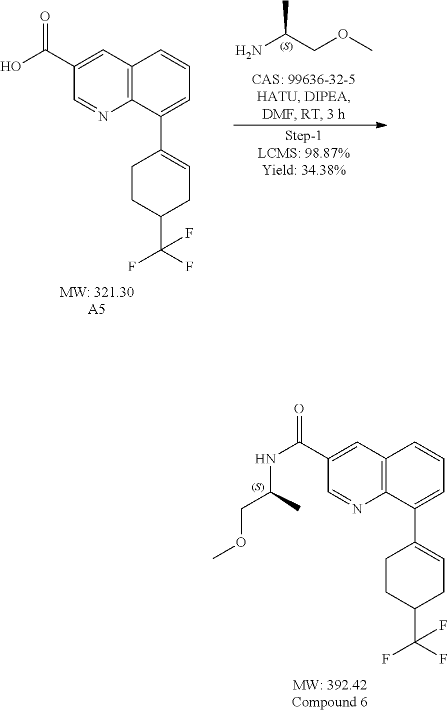

### Step-1:

A solution of 8-(4-(trifluoromethyl)cyclohex-1-en-1-yl)quinoline-3-carboxylic acid (A5, 0.15 g, 0.466 mmol, 1.0 eq), HATU (0.26 g, 0.700 mmol, 1.5 eq) and DIPEA (0.18 g, 1.400 mmol, 3.0 eq) in DMF (1.5 mL) was stirred for 30 min at 0° C. under nitrogen atmosphere, then added CAS: 99636-32-5 (0.045 g, 0.513 mmol, 1.1 eq). The resulting mixture was stirred for 3 h at room temperature. The reaction was monitored by TLC (using 100% Ethyl acetate as mobile phase) which confirmed that the reaction got completed after 3 h. The resulting reaction mixture was quenched with ice-cold water (10 mL) and extracted with EtOAc (3×15 mL). The combined organic layers were dried over Na₂SO₄ and concentrated under reduced pressure to afford 0.22 g crude. The obtained crude material was purified by flash column chromatography using silica gel (230-400 mesh) as stationary phase (70% Ethyl acetate in hexane as gradient) yielding N—((S)-1-methoxypropan-2-yl)-8-(4-(trifluoromethyl)cyclohex-1-en-1-yl)quinoline-3-carboxamide (Compound 6, 0.063 g, 0.160 mmol, Yield: 34.38%).

¹H NMR (DMSO-d6, 400 MHz): δ ppm, 9.24 (d, J=2.0 Hz, 1H), 8.80 (d, J=2.0 Hz, 1H), 8.60 (d, J=8.0 Hz, 1H), 8.02-7.99 (m, 1H), 7.64-7.61 (m, 2H), 5.87 (s, 1H), 4.29-4.23 (m, 1H), 3.47-3.43 (m, 1H), 3.34-3.31 (m, 1H), 3.30 (s, 3H), 2.80-2.77 (m, 1H), 2.72-2.68 (m, 2H), 2.50-2.46 (m, 1H), 2.32-2.24 (m, 1H), 2.10-2.07 (m, 1H), 1.73-1.67 (m, 1H), 1.19 (d, J=6.8 Hz, 3H).

¹H NMR (MeOD, 400 MHz): δ ppm, 9.24 (d, J=2.4 Hz, 1H), 8.77 (d, J=2.0 Hz, 1H), 7.97 (dd, J=7.6, 2.0 Hz, 1H), 7.70-7.63 (m, 2H), 5.87 (br s, 1H), 4.5-4.39 (m, 1H), 3.58-3.54 (m, 1H), 3.50-3.47 (m, 1H), 3.42 (s, 3H), 2.83-2.81 (m, 1H), 2.78-2.75 (m, 2H), 2.68-2.65 (m, 1H), 2.55-2.41 (m, 1H), 2.18-2.15 (m, 1H), 1.93-1.82 (m, 1H), 1.31 (d, J=6.8 Hz, 3H).

LCMS (Method A): 2.505 min, 98.87%, 254.0 nm, MS: ES+393.17 (M+1)

HPLC (Method A): 9.078 min, 98.54%, 254.0 nm

Chiral HPLC: 5.31 min, 50.73%, 240.0 nm; 5.63 min, 49.26%, 240.0 nm.

### Example 7—Synthesis of N—((R)-1-hydroxypropan-2-yl)-8-(4-(trifluoromethyl)cyclohex-1-en-1-yl)quinoline-3-carboxamide (Compound 7)

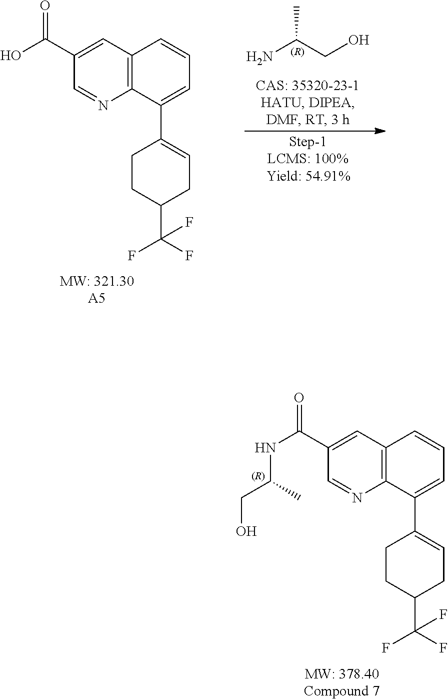

### Step-1:

To a solution of 8-(4-(trifluoromethyl)cyclohex-1-en-1-yl)quinoline-3-carboxylic acid (A5, 0.15 g, 0.467 mmol, 1.0 eq), HATU (0.35 g, 0.934 mmol, 2.0 eq) and DIPEA (0.18 g, 1.400 mmol, 3.0 eq) in DMF (1.5 mL) at 0° C. under nitrogen atmosphere stirred for 20 min, was added CAS: 35320-23-1 (0.042 g, 0.560 mmol, 1.2 eq) under Nitrogen. The resulting mixture was stirred for 3 h at room temperature. The reaction was monitored on TLC (using EtOAc 100% as mobile phase) which confirmed that the reaction got completed after 3 h. The resulting reaction mixture was quenched with ice-cold water (10 mL) and extracted with EtOAc (3×10 mL). The combined organic layers were dried over Na₂SO₄ and concentrated under reduced pressure to afford 0.3 g crude. The obtained crude material was purified by flash column chromatography using silica gel (230-400 mesh) as stationary phase (70% Ethyl acetate in hexane as gradient) yielding N—((R)-1-hydroxypropan-2-yl)-8-(4-(trifluoromethyl)cyclohex-1-en-1-yl)quinoline-3-carboxamide (Compound 7, 0.097 g, 0.256 mmol, Yield: 54.91%).

¹H NMR (DMSO-d6, 400 MHz): δ ppm, 9.25 (d, J=2.4 Hz, 1H), 8.80 (d, J=2.4 Hz, 1H), 8.47 (d, J=8.0 Hz, 1H), 8.01-7.98 (m, 1H), 7.64-7.61 (m, 2H), 5.87 (br s, 1H), 4.79 (t, J=6.0 Hz, 1H), 4.12-4.01 (m, 1H), 3.54-3.48 (m, 1H), 3.42-3.38 (m, 1H), 2.89-2.77 (m, 1H), 2.73-2.68 (m, 2H), 2.50-2.46 (m, 1H), 2.33-2.25 (m, 1H), 2.09-2.07 (m, 1H), 1.73-1.67 (m, 1H), 1.18 (d, J=6.8 Hz, 3H).

¹H NMR (MeOD, 400 MHz): δ ppm, 9.25 (d, J=2.0 Hz, 1H), 8.78 (d, J=2.0 Hz, 1H), 7.97 (d, J=7.6 Hz, 1H), 7.69-7.62 (m, 2H), 5.86 (br s, 1H), 4.30-4.25 (m, 1H), 3.67 (d, J=5.6 Hz, 1H), 2.88-2.65 (m, 3H), 2.55-2.51 (m, 1H), 2.40-2.37 (m, 1H), 2.17-2.15 (m, 1H), 1.89-1.85 (m, 1H), 1.31 (d, J=6.8 Hz, 3H).

LCMS (Method A): 2.237 min, 100%, 254.0 nm, MS: ES+379.1 (M+1)

HPLC (Method A): 7.930 min, 100%, 254.0 nm

Chiral HPLC: 6.17 min, 49.36%, 6.99 min, 50.32%, 240.0 nm

### Example 8—Synthesis of N-((1-methyl-1H-imidazol-2-yl)methyl)-8-(4-(trifluoromethyl)cyclohex-1-en-1-yl)quinoline-3-carboxamide (Compound 8)

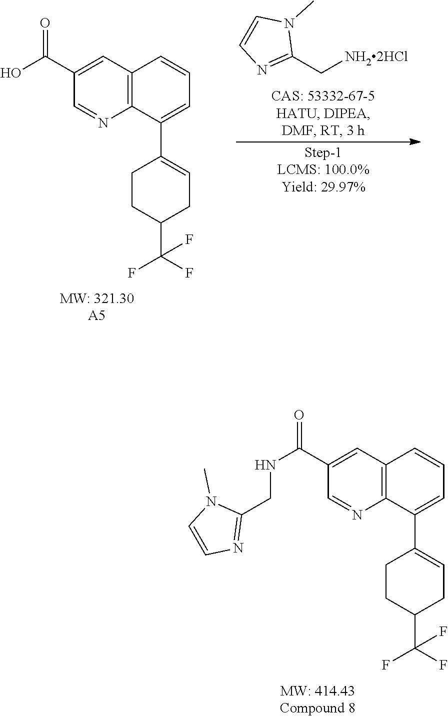

### Step-1:

To a solution of 8-(4-(trifluoromethyl)cyclohex-1-en-1-yl)quinoline-3-carboxylic acid (A5, 0.15 g, 0.466 mmol, 1.0 eq), HATU (0.26 g, 0.700 mmol, 1.5 eq) and DIPEA (0.18 g, 1.400 mmol, 3.0 eq) in DMF (1.5 mL) at 0° C. under nitrogen atmosphere stirred for 10 min, was added CAS: 53332-67-5 (0.094 g, 0.513 mmol, 1.1 eq) under Nitrogen. The resulting mixture was stirred for 3 h at room temperature. The reaction was monitored by TLC (using MeOH:DCM; 1.0:9.0 as mobile phase) which confirmed that the reaction got completed after 3 h. The resulting reaction mixture was quenched with ice-cold water (10 mL) and extracted with EtOAc (3×15 mL). The combined organic layers were dried over Na₂SO₄ and concentrated under reduced pressure to afford 0.223 g crude. The obtained crude material was purified by flash column chromatography using silica gel (230-400 mesh) as stationary phase (5% Methanol in DCM) yielding N-((1-methyl-1H-imidazol-2-yl)methyl)-8-(4-(trifluoromethyl)cyclohex-1-en-1-yl)quinoline-3-carboxamide (Compound 8, 0.058 g, 0.139 mmol, Yield: 29.97%)

¹H NMR (DMSO-d₆, 400 MHz): δ ppm, 9.30-9.27 (m, 2H), 8.85 (s, 1H), 8.00-7.97 (m, 1H), 7.64-7.62 (m, 2H), 7.11 (s, 1H), 6.82 (s, 1H), 5.86 (br s, 1H), 4.60 (d, J=5.2 Hz, 2H), 3.69 (s, 3H), 2.71-2.67 (m, 3H), 2.50-2.45 (m, 1H), 2.25-2.22 (m, 1H), 2.09-2.06 (m, 1H), 1.71-1.65 (m, 1H)

¹H NMR (MeOD, 400 MHz): δ ppm, 9.27 (d, J=2.4 Hz, 1H), 8.81 (d, J=2.0 Hz, 1H), 7.96 (dd, J=7.6, 2.0 Hz, 1H), 7.69-7.62 (m, 2H), 7.09 (d, J=1.2 Hz, 1H), 6.93 (d, J=1.6 Hz, 1H), 5.86 (br s, 1H), 4.75 (s, 2H), 3.81 (s, 3H), 2.81-2.64 (m, 3H), 2.55-2.50 (m, 1H), 2.40-2.33 (m, 1H), 2.17-2.15 (m, 1H), 1.92-1.84 (m, 1H).

LCMS (Method A): 1.892 min, 100.0%, 210.0 nm, MS: ES+415.1 (M+1)

HPLC (Method A): 8.081 min, 98.59%, 210.0 nm

### Example 9—Synthesis of N—((R)-1-methoxypropan-2-yl)-8-(4-(trifluoromethyl)cyclohex-1-en-1-yl)quinoline-3-carboxamide (Compound 9)

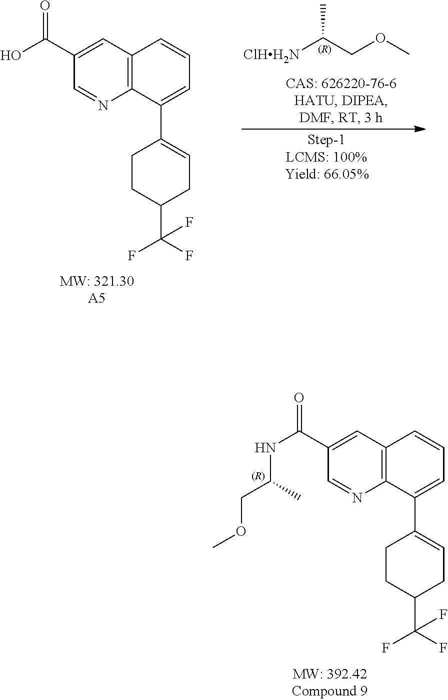

### Step-1:

To a solution of 8-(4-(trifluoromethyl)cyclohex-1-en-1-yl)quinoline-3-carboxylic acid (A5, 0.150 g, 0.466 mmol, 1.0 eq), HATU (0.26 g, 0.700 mmol, 1.5 eq) and DIPEA (0.18 g, 1.400 mmol, 3.0 eq) in DMF (1.5 mL) at 0° C. under nitrogen atmosphere stirred for 15 min, was added CAS: 626220-76-6 (0.064 g, 0.513 mmol, 1.1 eq) under Nitrogen. The resulting mixture was stirred for 3 h at room temperature. The reaction was monitored by TLC (using 100% EtOAc as mobile phase) which confirmed that the reaction got completed after 3 h. The resulting reaction mixture was quenched with ice-cold water (10 mL) and extracted with EtOAc (3×15 mL). The combined organic layers were dried over Na₂SO₄ and concentrated under reduced pressure to afford 0.180 g crude. The obtained crude material was purified by trituration with ACN yielding N—((R)-1-methoxypropan-2-yl)-8-(4-(trifluoromethyl)cyclohex-1-en-1-yl)quinoline-3-carboxamide (Compound 9, 0.121 g, 0.308 mmol, Yield: 66.05%).

¹H NMR (DMSO-d6, 400 MHz): δ ppm, 9.24 (d, J=2.4 Hz, 1H), 8.79 (d, J=2.4 Hz, 1H), 8.60 (d, J=8.0 Hz, 1H), 8.01-7.99 (m, 1H), 7.64-7.63 (m, 2H), 5.87 (br s, 1H), 4.29-4.23 (m, 1H), 3.47-3.43 (m, 1H), 3.35-3.31 (m, 1H) 3.29 (s, 3H), 2.80-2.77 (m, 1H), 2.72-2.68 (m, 2H), 2.47-2.46 (m, 1H), 2.32-2.24 (m, 1H), 2.09-2.07 (m, 1H), 1.73-1.67 (m, 1H), 1.19 (d, J=6.8 Hz, 3H).

¹H NMR (MeOD, 400 MHz): δ ppm, 9.23 (d, J=2.4 Hz, 1H), 8.75 (d, J=2.0 Hz, 1H), 7.96 (dd, J=7.6, 2.0 Hz, 1H), 7.68-7.62 (m, 2H), 5.86 (br s, 1H), 4.43-4.39 (m, 1H), 3.57-3.50 (m, 1H), 3.49-3.46 (m, 1H), 3.42 (s, 3H), 2.80-2.71 (m, 1H), 2.67-2.55 (m, 2H), 2.52-2.50 (m, 1H), 2.40-2.33 (m, 1H), 2.18-2.14 (m, 1H), 1.92-1.81 (m, 1H), 1.31 (d, J=6.8 Hz, 3H).

LCMS (Method A): 2.480 min, 100.0%, 254.0 nm, MS: ES+393.17 (M+1)

HPLC (Method A): 9.068 min, 98.51, 210.0 nm

Chiral HPLC: 4.64 min and 4.93 min, 49.45% and 50.54%, 240.0 nm

### Example 10—Synthesis of N-((4-methyl-1H-pyrazol-5-yl)methyl)-8-(4-(trifluoromethyl)cyclohex-1-en-1-yl)quinoline-3-carboxamide (Compound 10)

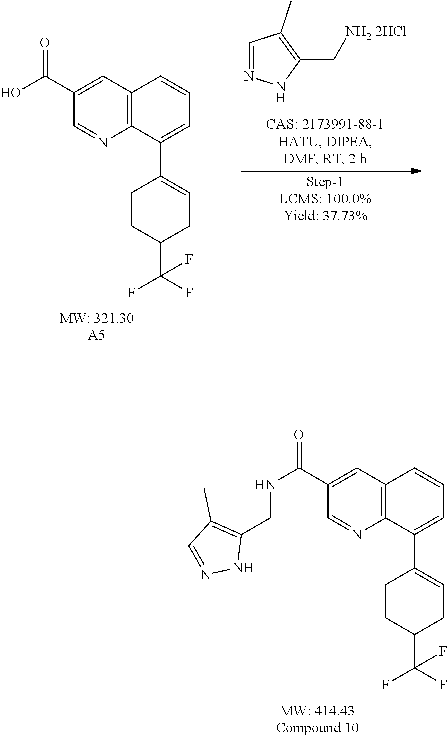

### Step-1:

To a solution of 8-(4-(trifluoromethyl)cyclohex-1-en-1-yl)quinoline-3-carboxylic acid (A5, 0.15 g, 0.466 mmol, 1.0 eq), HATU (0.26 g, 0.700 mmol, 1.5 eq) and DIPEA (0.18 g, 1.400 mmol, 3.0 eq) in DMF (1.5 mL) at 0° C. under nitrogen atmosphere stirred for 15 min, was added CAS: 2173991-88-1 (0.094 g, 0.513 mmol, 1.1 eq) under Nitrogen. The resulting mixture was stirred for 2 h at room temperature. The reaction was monitored by TLC (using MeOH:DCM; 1.0:9.0 as mobile phase) which confirmed that the reaction got completed after 2 h. The resulting reaction mixture was quenched with ice-cold water (10 mL) and extracted with EtOAc (3×15 mL). The combined organic layers were dried over Na₂SO₄ and concentrated under reduced pressure to afford 0.194 g crude. The obtained crude material was purified by flash column chromatography using silica (230-400 mesh) as stationary phase (70% Ethyl acetate in hexane as gradient) yielding N-((4-methyl-1H-pyrazol-5-yl)methyl)-8-(4-(trifluoromethyl)cyclohex-1-en-1-yl)quinoline-3-carboxamide (Compound 10, 0.073 g, 0.176 mmol, Yield: 37.73%).

¹H NMR (DMSO-d₆, 400 MHz): δ ppm, 12.54, 12.38 (s, 1H), 9.26 (d, J=2.4 Hz, 1H), 9.18-9.09 (m, 1H), 8.83 (d, J=2.4 Hz, 1H), 7.99-7.97 (m, 1H), 7.64-7.60 (m, 2H), 7.46 (br s, 1H), 5.86 (br s, 1H), 4.52 (d, J=4.8 Hz, 2H), 2.84-2.77 (m, 2H), 2.28-2.24 (m, 1H), 2.09-2.06 (m, 1H), 2.02 (s, 3H), 1.74-1.65 (m, 1H). Note: Aliphatic 2 proton merged with DMSO solvent peaks which is clearly visible in MeOD NMR.

¹H NMR (MeOD, 400 MHz): δ ppm, 9.26 (d, J=2.4 Hz, 1H), 8.77 (d, J=2.0 Hz, 1H), 7.95 (d, J=7.6 Hz, 1H), 7.68-7.62 (m, 2H), 7.42 (br s, 1H), 5.86 (br s, 1H), 4.68 (s, 2H), 2.77-2.74 (m, 1H), 2.64 (d, J=2.8 Hz, 2H), 2.55-2.50 (m, 1H), 2.40-2.33 (m, 1H), 2.19-2.10 (m, 4H), 1.92-1.81 (m, 1H).

LCMS (Method A): 2.331 min, 100.0%, 210.0 nm, MS: ES+415.1 (M+1)

HPLC (Method A): 8.273 min, 98.67%, 210.0 nm

### Example 11—Synthesis of N-(oxazol-2-ylmethyl)-8-(4-(trifluoromethyl)cyclohex-1-en-1-yl)quinoline-3-carboxamide (Compound 11)

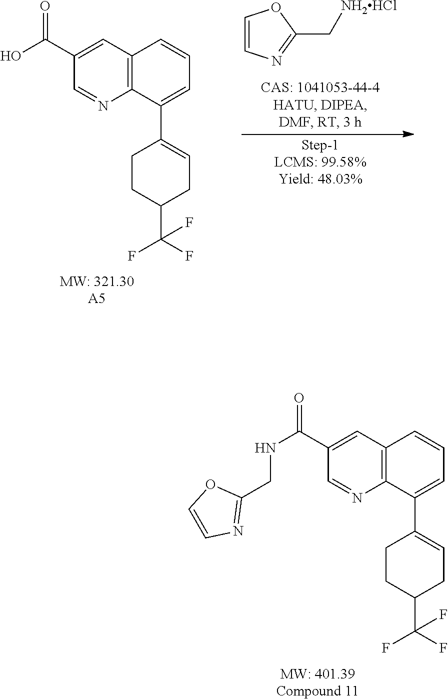

### Step-1:

To a solution 8-(4-(trifluoromethyl)cyclohex-1-en-1-yl)quinoline-3-carboxylic acid (A5, 0.15 g, 0.466 mmol, 1.0 eq), HATU (0.35 g, 0.933 mmol, 2.0 eq) and DIPEA (0.24 g, 1.867 mmol, 4.0 eq) in DMF (1.5 mL) at 0° C. under nitrogen atmosphere stirred for 20 min, was added CAS: 1041053-44-4 (0.075 g, 0.560 mmol, 1.2 eq) under Nitrogen. The resulting mixture was stirred for 3 h at room temperature. The reaction was monitored by TLC (using EtOAc:Hexane; 8:2 as mobile phase) which confirmed that the reaction got completed after 3 h. The resulting reaction mixture was quenched with ice-cold water (10 mL) and extracted with EtOAc (3×15 mL). The combined organic layers were dried over Na₂SO₄ and concentrated under reduced pressure to afford 0.2 g crude. The obtained crude material was purified by flash column chromatography using silica gel (230-400 mesh) as stationary phase (75% Ethyl acetate in hexane as gradient) yielding N-(oxazol-2-ylmethyl)-8-(4-(trifluoromethyl)cyclohex-1-en-1-yl)quinoline-3-carboxamide (Compound 11, 0.090 g, 0.224 mmol, Yield: 48.03%).

¹H NMR (DMSO-d6, 400 MHz): δ ppm, 9.52 (t, J=5.6 Hz, 1H), 9.27 (d, J=2.4 Hz, 1H), 8.86 (d, J=2.0, Hz, 1H), 8.09 (d, J=0.8 Hz, 1H), 8.02 (dd, J=6.8, 2.8 Hz, 1H), 7.67-7.62 (m, 2H), 7.19 (d, J=0.8 Hz, 1H), 5.87 (br s, 1H), 4.67 (d, J=5.6 Hz, 2H), 2.85-2.78 (m, 1H), 2.71-2.67 (m, 2H), 2.33-2.25 (m, 1H), 2.10-2.07 (m, 1H), 1.74-1.67 (m, 1H). Note: CF3-CH proton merged with DMSO solvent peak which is clearly visible in MeOD NMR.

¹H NMR (MeOD, 400 MHz): δ ppm, 9.29 (d, J=2.4 Hz, 1H), 8.82 (d, J=2.4 Hz, 1H), 7.98 (dd, J=8.0, 2.0 Hz, 1H), 7.93 (d, J=0.4 Hz, 1H), 7.71-7.64 (m, 2H), 7.18 (s, 1H), 5.87 (br s, 1H), 4.80 (s, 2H), 2.83-2.76 (m, 1H), 2.68-2.64 (m, 2H), 2.55-2.51 (m, 1H), 2.41-2.37 (m, 1H), 2.18-2.15 (m, 1H), 1.92-1.86 (m, 1H) Note: —CONH proton exchanged in MeOD NMR.

LCMS (Method A): 2.341 min, 99.58%, 220.0 nm, MS: ES+402.07 (M+1)

HPLC (Method A): 8.385 min, 98.13%, 210.0 nm

### Example 12—Chiral Separation of N-(oxazol-2-ylmethyl)-8-(4-(trifluoromethyl)cyclohex-1-en-1-yl)quinoline-3-carboxamide (Compounds 12 and 13)

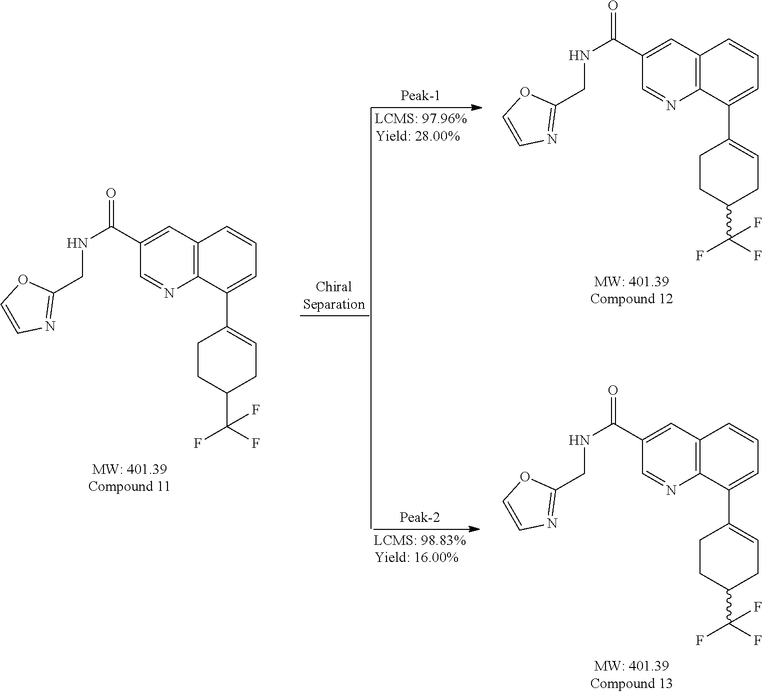 [SMILES: O=C(NCc1ncco1)c1cnc2c(C3=CCC(C(F)(F)F)CC3)cccc2c1 ; O=C(NCc1ncco1)c1cnc2c(C3=CCC(C(F)(F)F)CC3)cccc2c1 ; O=C(NCc1ncco1)c1cnc2c(C3=CCC(C(F)(F)F)CC3)cccc2c1]

### Procedure:

Compound 11 (0.025 g) racemic was subjected to chiral SFC purification on (CHIRALPAK IG 250×10 mm 5 μm) column where 2 peaks were separated: Peak 1 (0.007 g Yield=28.00%) as Compound 12 and Peak 2 (0.004 g, Yield=16.00%) as Compound 13.

COLUMN ID: CHIRALPAK IG, 250×10 mm, 5 μm

MOBILE PHASE A: Liq.CO2

MOBILE PHASE B: 0.1% Methanolic Ammonia in IPA-ACN (70-30)

FLOW RATE (ML/MIN): 14

INSTRUMENT ID: SFC INVESTIGATOR

METHOD: TIME: FLOW: % A: % B (0:14:60:40), (7:14:60:40)

### Compound 12:

¹H NMR (DMSO-d₆, 400 MHz): δ ppm, 9.53 (t, J=5.2 Hz, 1H), 9.28 (d, J=1.6 Hz, 1H), 8.86 (d, J=1.6 Hz, 1H), 8.09 (s, 1H), 8.02 (d, J=5.6 Hz, 1H), 7.66 (br s, 2H), 7.19 (s, 1H), 5.88 (s, 1H), 4.67 (d, J=5.2 Hz, 2H), 2.78-2.67 (m, 3H), 2.33-2.29 (m, 1H), 2.10-2.07 (m, 1H), 1.72-1.68 (m, 1H)

LCMS (Method A): 2.368 min, 97.96%, 254.0 nm, MS: ES+402.2 (M+1)

HPLC (Method A): 8.461 min, 98.53%, 254.0 nm

### Compound 13:

¹H NMR (DMSO-d₆, 400 MHz): δ ppm, 9.53 (t, J=5.2 Hz, 1H), 9.28 (d, J=1.6 Hz, 1H), 8.86 (d, J=1.6 Hz, 1H), 8.09 (s, 1H), 8.02 (d, J=5.6 Hz, 1H), 7.66-7.63 (m, 2H), 7.20 (s, 1H), 5.88 (s, 1H), 4.67 (d, J=5.6 Hz, 2H), 2.81-2.68 (m, 3H), 2.33-2.25 (m, 1H), 2.10-2.07 (m, 1H), 1.75-1.67 (m, 1H)

LCMS (Method A): 2.367 min, 98.83%, 254 nm, MS: ES+402.1 (M+1)

HPLC (Method A): 8.481 min, 99.19%, 254.0 nm

### Example 13—Synthesis of 3-(methylsulfinyl)-8-(4-(trifluoromethyl)cyclohex-1-en-1-yl)quinoline (Compound 14)

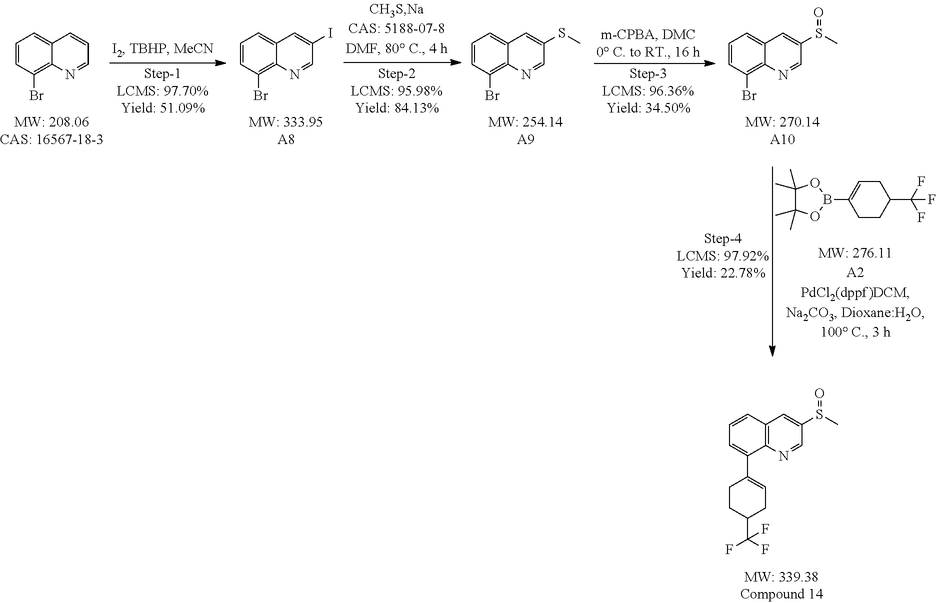 [SMILES: Brc1cccc2cccnc12 ; Brc1cccc2cc(I)cnc12 ; CSc1cnc2c(Br)cccc2c1 ; CS(=O)c1cnc2c(Br)cccc2c1 ; CC1(C)OB(C2=CCC(C(F)(F)F)CC2)OC1(C)C ; CS(=O)c1cnc2c(C3=CCC(C(F)(F)F)CC3)cccc2c1]

### Step-1:

To a stirred solution of 8-bromoquinoline CAS: 16567-18-3 (5.0 g, 23.96 mmol, 1.0 eq) in Acetonitrile (50 mL) was added Iodine (12.16 g, 47.93 mmol, 2.0 eq) and TBHP (70% aqueous sol.) followed by addition of CAS: 75-91-2 (30.93 mL, 240.3 mmol, 10.0 eq). The resulting mixture was stirred at 80° C. for 16 h. The reaction was monitored on TLC (using EtOAc:Hexane; 1.0:9.0 as mobile phase) which confirmed that the reaction got completed after 16 h of stirring at 80° C. The resulting reaction mixture was diluted with water (50 mL) and extracted with EtOAc (3×50 mL). The combined organic layers were dried over Na₂SO₄ and concentrated under reduced pressure to afford 5.5 g crude. The obtained crude material was purified by flash column chromatography using silica (230-400 mesh) as stationary phase (1% EtOAc in Hexane) yielding 8-bromo-3-iodoquinoline (A8, 4.1 g, 12.27 mmol, Yield: 51.09%).

¹H NMR (DMSO-d₆, 400 MHz): 9.16 (d, J=2.0 Hz, 1H), 8.97 (d, J=2.0 Hz, 1H), 8.18 (d, J=7.4 Hz, 1H), 7.98 (d, J=7.6 Hz, 1H), 7.56 (t, J=7.6 Hz, 8 Hz, 1H)

LCMS (Method A): 2.516 min, 97.70%, 254.0 nm, MS: ES-336.0 (M+2)

### Step-2:

To a stirred solution of 8-bromo-3-iodoquinoline (A8, 4.0 g, 11.98 mmol, 1.0 eq) in Toluene (40 mL) was added CAS: 5188-07-8 (1.17 g, 16.77 mmol, 1.4 eq) under N₂. The resulting mixture was stirred at 80° C. for 3 h. The reaction was monitored on TLC (using EA:n-hexane; 1.0:9.0 as mobile phase) which confirmed that the reaction got completed after 3 h of stirring at 80° C. The resulting reaction mixture was quenched with cold water (40 mL) to precipetated out the crude which was filtered off and washed with 50 mL of water and dried under reduced pressure to afford 2.9 g crude. The obtained crude material was triturated by n-hexane yielding 8-bromo-3-(methylthio) quinoline (A9, 2.5 g, 9.84 mmol, Yield: 84.13%).

¹H NMR (DMSO-d6, 400 MHz): δ ppm, 8.90 (d, J=2.4 Hz, 1H), 8.24 (d, J=2.0 Hz, 1H), 8.04 (dd, J=7.6, 1.2 Hz, 1H), 7.95 (dd, J=8.2 Hz, 0.8 Hz, 1H), 7.52 (t, J=8.0 Hz, 1H), 2.66 (s, 3H).

LCMS (Method A): 2.295 min, 95.98%, 254.0 nm, MS: ES+256.10 (M+2)

### Step-3:

To a stirred solution of 8-bromo-3-(methylthio) quinoline (A9, 1.5 g, 5.90 mmol, 1.0 eq) in DCM (15 mL) was added m-CPBA (1.52 g, 8.85 mmol, 1.5 eq) at 0° C. under N₂. The resulting mixture was stirred at room temperature for 2 h. The reaction was monitored on TLC (using EtOAc:Hex; 7.0:3.0 as mobile phase) which confirmed that the reaction got completed after 2 h of stirring at room temperature. The resulting reaction mixture was quenched with cold water (10 mL) and extracted with DCM (3×20 mL). The combined organic layers were dried over Na₂SO₄ and concentrated under reduced pressure to afford 1.5 g crude. The obtained crude material was purified by flash column chromatography using silica (230-400 mesh) as stationary phase (2% MeOH in DCM) yielding 8-bromo-3-(methylsulfinyl)quinoline (A10, 0.55 g, 2.04 mmol, Yield: 34.50%).

¹H NMR (DMSO-d6, 400 MHz): δ ppm, 9.23 (d, J=2.0 Hz, 1H), 8.80 (d, J=2.4 Hz, 1H), 8.29 (dd, J=7.6, 2.0 Hz, 1H), 8.23 (dd, J=8.0 Hz, 1.2 Hz, 1H), 7.65 (t, J=8.0 Hz, 1H), 2.97 (s, 3H).

LCMS (Method A): 1.553 min, 96.36%, 254.0 nm, MS: ES+272.00 (M+2)

### Step-4:

To a stirred solution of 8-bromo-3-(methylsulfinyl)quinoline (A10, 0.25 g, 0.925 mmol, 1.0 eq), 4,4,5,5-tetramethyl-2-(4-(trifluoromethyl)cyclohex-1-en-1-yl)-1,3,2-dioxaborolane (A2, 0.38 g, 1.388 mmol, 1.5 eq) and Na₂CO₃ (0.29 g, 2.776 mmol, 3.0 eq) in Dioxane (2.0 mL) and water (0.5 mL) was added Pd(dppf)Cl₂·DCM (0.075 g, 0.092 mmol, 0.1 eq) under N₂. The resulting mixture was stirred at 100° C. for 3 h. The reaction was monitored by TLC (using EA:Hex; 7.0:3.0 as mobile phase) which confirmed that the reaction got completed after 3 h of stirring at 100° C. The resulting reaction mixture was diluted with water (10 mL) and extracted with EtOAc (3×20 mL). The combined organic layers were dried over Na₂SO₄ and concentrated under reduced pressure to afford 0.4 g crude. The obtained crude material was purified by flash column chromatography using silica gel (230-400 mesh) as stationary phase (75% Ethyl acetate in n-hexane) yielding 3-(methylsulfinyl)-8-(4-(trifluoromethyl)cyclohex-1-en-1-yl)quinoline (Compound 14, 0.07 g, 0.206 mmol, Yield: 22.78%).

¹H NMR (DMSO-d₆, 400 MHz): δ ppm, 9.11 (d, J=2.0 Hz, 1H), 8.70 (d, J=2.0 Hz, 1H), 8.10-8.07 (m, 1H), 7.70-7.67 (m, 2H), 5.86 (br s, 1H), 2.94 (s, 3H), 2.77-2.67 (m, 3H), 2.33-2.28 (m, 1H), 2.10-2.07 (m, 1H), 1.72-1.67 (m, 1H). Note: CF3-CH merged with DMSO solvent peak which is clearly observed in MeOD NMR.

¹H NMR (MeOD, 400 MHz): δ ppm, 9.10 (d, J=1.6 Hz, 1H), 8.69 (d, J=2.4 Hz, 1H), 8.01 (dd, J=7.6, 2.4 Hz, 1H), 7.74-7.68 (m, 2H), 5.87 (br s, 1H), 3.01 (s, 3H), 2.83-2.78 (m, 1H), 2.72-2.61 (m, 2H), 2.55-2.51 (m, 1H), 2.40-2.33 (m, 1H), 2.18-2.15 (m, 1H), 1.91-1.82 (m, 1H).

Note: Minor aliphatic impurity observed.

LCMS (Method A): 2.357 min, 97.92%, 254.0 nm, MS: ES+340.06 (M+1)

HPLC (Method A): 8.493 min, 98.25%, 254.0 nm

### Example 14—Synthesis of 8-(4-(trifluoromethyl)cyclohex-1-en-1-yl)quinoline-3-carboxylic Acid (Compound 15)

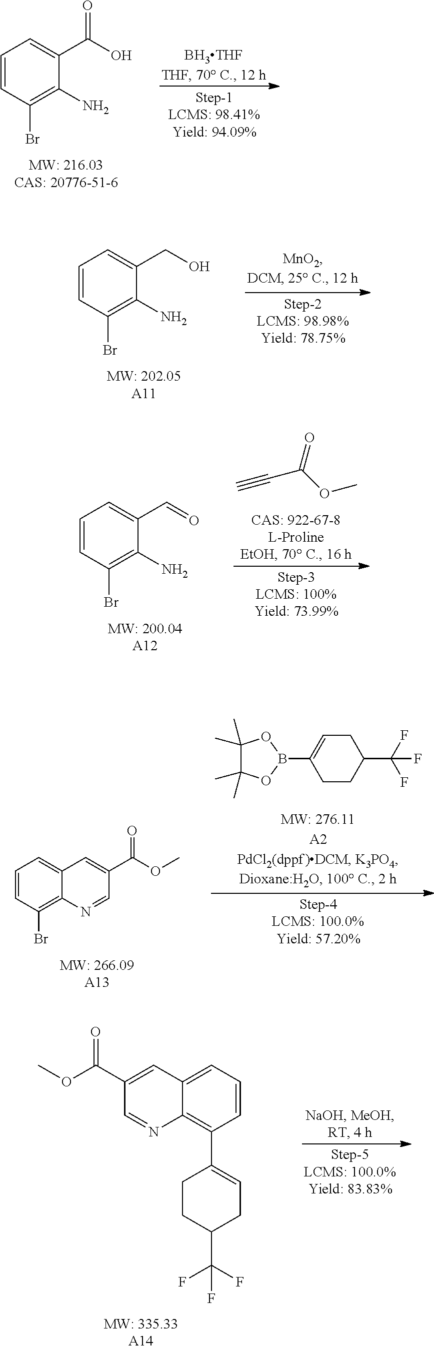

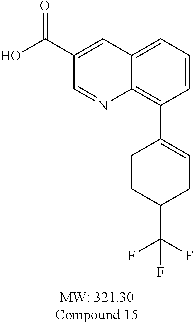 [SMILES: O=C(O)c1cnc2c(C3=CCC(C(F)(F)F)CC3)cccc2c1]

### Step-1:

To a solution of 2-amino-3-bromobenzoic acid (CAS: 20776-51-6) (5.0 g, 23.145 mmol, 1.0 eq) in THF (20 mL) was added BH₃.THF (1M in THF, 81.0 mL, 81.007 mmol, 3.5 eq) at 0° C. The mixture was stirred at 70° C. for 12 h. The reaction was monitored on TLC (using EtOAc as mobile phase) which confirmed that the reaction got completed after 12 h of stirring at 70° C. Then the reaction mixture was quenched with MeOH, filtered and concentrated under reduced pressure to obtained a crude. The residue was stirred with water (100 mL) and filtered. The residue was dissolved in DCM and dried over Na₂SO₄ and evaporated under reduced pressure to give (2-Amino-3-bromo-phenyl) methanol (A11, 4.4 g, 21.78 mmol, 94.09% yield) as a pale brown solid.

¹H NMR (CDCl₃, 400 MHz): δ ppm, 7.41 (dd, J=8.4, 1.6 Hz, 1H), 7.02 (d, J=7.6 Hz, 1H), 6.60 (t, J=8.0 Hz, 1H), 4.68 (s, 2H), 3.65 (br s, 3H). Note: Minor aliphatic impurities observed

LCMS (Method A): 1.667 min, 98.41%, 254.0 nm, MS: ES+202.0 (M), 204.0 (M+2)

### Step-2:

To a solution of (2-Amino-3-bromo-phenyl) methanol (A11, 4.4 g, 21.78 mmol, 1 eq) in DCM (40 mL) was added MnO₂ (18.93 g, 232.40 mmol, 10 eq). The mixture was stirred at room temperature for 12 h. The reaction was monitored on TLC (using EA:Hex; 6.0:4.0 as mobile phase) which confirmed that the reaction got completed after 12 h of stirring at room temperature. The reaction mixture was filtered and concentrated under reduced pressure to give 2-amino-3-bromobenzaldehyde (A12, 3.5 g, 17.50 mmol, Yield: 78.75%).

¹H NMR (CDCl₃, 400 MHz): δ ppm, 9.85 (s, 1H), 7.64 (dd, J=7.6, 1.6 Hz, 1H), 7.51 (dd, J=7.6, 1.6 Hz, 1H), 6.70 (t, J=7.6 Hz, 1H) Note: —NH₂ protons not observed.

LCMS (Method A): 1.966 min, 98.98%, 254.0 nm, MS: ES+200.0 (M), 201.99 (M+2)

### Step-3:

To a solution of (A12, 380 mg, 1.8996 mmol, 1 eq) in EtOH (4 mL) was added L-proline (109 mg, 0.9498 mmol, 0.5 eq) and CAS 922-67-80 (0.22 mL, 2.4695 mmol, 1.3 eq). The mixture was stirred at 80° C. for 16 h. The reaction was monitored on TLC (using EtOAc:Hex as mobile phase) which confirmed that the reaction got completed after 16 h of stirring at 80° C. The reaction mixture was concentrated under reduced pressure to give a residue. The residue was purified by column chromatography using silica gel (60-120 mesh) as stationary phase (100% Ethyl acetate in hexane as gradient) to give Methyl 8-bromoquinoline-3-carboxylate (A13, 374 mg, 1.41 mmol, 73.99% yield).

¹H NMR (CDCl₃, 400 MHz): δ ppm, 9.59 (d, J=2.0 Hz, 1H), 8.89 (d, J=2.0 Hz, 1H), 8.20 (dd, J=7.6, 1.6 Hz, 1H), 7.95 (dd, J=8.4, 1.2 Hz, 1H), 7.52 (t, J=8.0 Hz, 1H), 4.06 (s, 3H).

LCMS (Method B): 2.61 min, 100%, 254.0 nm, MS: ES+268.02 (M+2)

### Step-4:

To a stirred solution of 4,4,5,5-tetramethyl-2-(4-(trifluoromethyl)cyclohex-1-en-1-yl)-1,3,2-dioxaborolane (A2, 1.14 g, 4.133 mmol, 1.1 eq), methyl 8-bromoquinoline-3-carboxylate (A13, 1.0 g, 3.758 mmol, 1.0 eq) and K₃PO₄ (1.59 g, 7.516 mmol, 2.0 eq) in Dioxane (7 mL) and water (3 mL) was added Pd(dppf)Cl₂·DCM (0.306 g, 0.375 mmol, 0.1 eq) under N₂. The resulting mixture was stirred at 100° C. for 2 h. The reaction was monitored on TLC (using EtOAc:Hexane; 2.0:8.0 as mobile phase) which confirmed that the reaction got completed after 2 h of stirring at 100° C. The resulting reaction mixture was diluted with water (10 mL) and extracted with EtOAc (3×20 mL). The combined organic layer were dried over Na₂SO₄ and concentrated under reduced pressure to afford 1.2 g crude. The obtained crude material was purified by flash column chromatography using silica gel (230-400 mesh) as stationary phase (55% EtOAc in Hexane) yielding methyl 8-(4-(trifluoromethyl)cyclohex-1-en-1-yl)quinoline-3-carboxylate (A14, 0.72 g, 2.147 mmol, Yield: 57.20%).

¹H NMR (DMSO-d₆, 400 MHz): δ ppm, 9.29 (d, J=2.0 Hz, 1H), 9.00 (d, J=2.0 Hz, 1H), 8.13 (dd, J=7.6, 1.6 Hz, 1H), 7.71-7.64 (m, 2H), 5.86 (br s, 1H), 3.95 (s, 3H), 2.80-2.77 (m, 1H), 2.69-2.66 (m, 3H), 2.31-2.27 (m, 1H), 2.09-2.06 (m, 1H), 1.72-1.66 (m, 1H).

LCMS (Method A): 2.903 min, 100.0%, 210.0 nm, MS: ES+336.06 (M+1)

### Step-5:

A solution of methyl 8-(4-(trifluoromethyl)cyclohex-1-en-1-yl)quinoline-3-carboxylate (A14, 1.0 g, 2.982 mmol, 1.0 eq) and NaOH (0.59 g, 14.912 mmol, 5.0 eq) in MeOH:H₂O (10 mL, 7:3) was prepared at 0° C. under nitrogen atmosphere. The resulting mixture was stirred for 4 h at room temperature. The reaction was monitored by TLC (using EtOAc:Hexane; 6.0:4.0 as mobile phase) which confirmed that the reaction got completed after 4 h. The resulting reaction mixture was concentrated under reduced pressure and obtained residue was poured onto ice-water then filtered off to afford the desired product 8-(4-(trifluoromethyl)cyclohex-1-en-1-yl)quinoline-3-carboxylic acid (Compound 15, 0.8 g, 2.489 mmol, Yield: 83.83%).

¹H NMR (DMSO-d6, 400 MHz): δ ppm, 13.54 (br s, 1H), 9.29 (d, J=2.4 Hz, 1H), 8.98 (d, J=2.4, Hz, 1H), 8.12 (dd, J=7.6, 2.0 Hz, 1H), 7.71-7.64 (m, 2H), 5.87 (br s, 1H), 2.84-2.67 (m, 3H), 2.47-2.42 (m, 1H), 2.33-2.24 (m, 1H), 2.10-2.07 (m, 1H), 1.75-1.65 (m, 1H).

¹H NMR (MeOD, 400 MHz): δ ppm, 9.49 (d, J=2.0 Hz, 1H), 9.42 (d, J=2.0 Hz, 1H), 8.29 (dd, J=8.4, 1.2 Hz, 1H), 8.00 (dd, J=7.2, 1.2 Hz, 1H), 7.91 (t, J=8.0 Hz, 1H), 6.02 (br s, 1H), 2.78-2.71 (m, 2H), 2.68-2.57 (m, 2H), 2.47-2.40 (m, 1H), 2.25-2.21 (m, 1H), 1.98-1.88 (m, 1H).

Note: —COOH proton exchanged with MeOD.

LCMS (Method A): 2.475 min, 100.0%, 254.0 nm, MS: ES+322.06 (M+1)

HPLC (Method A): 4.280 min, 99.77%, 254.0 nm

### Example 15—Synthesis of 3-(methylsulfonyl)-8-(4-(trifluoromethyl)cyclohex-1-en-1-yl)quinoline (Compound 16)

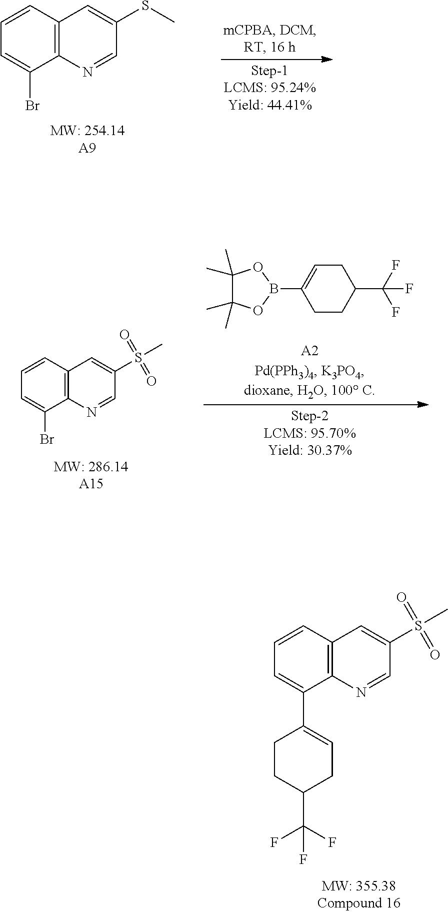 [SMILES: CSc1cnc2c(Br)cccc2c1 ; CC1(C)OB(C2=CCC(C(F)(F)F)CC2)OC1(C)C ; CS(=O)(=O)c1cnc2c(Br)cccc2c1 ; CS(=O)(=O)c1cnc2c(C3=CCC(C(F)(F)F)CC3)cccc2c1]

### Step-1:

To a stirred solution of 5-bromo-3-(methylthio) quinoline (A9, 1.0 g, 3.934 mmol, 1.0 eq), in DCM (25 mL) was added m-chloroperbenzoic acid (2.10 g, 11.80 mmol, 3.0 eq) under N₂. The resulting mixture was stirred at room temperature for 16 h. The reaction was monitored by TLC (using EA:Hex; 1.0:1.0 as mobile phase) which confirmed that the reaction got completed after 16 h of stirring at room temperature. The resulting reaction mixture was diluted with saturated aqueous sodium bicarbonate solution (200 mL) and extracted with DCM (2×100 mL). The combined organic layers were dried over Na₂SO₄ and concentrated under reduced pressure to afford 0.65 g crude. The obtained crude material was purified by flash column chromatography using silica gel (230-400 mesh) as stationary phase (55% Ethyl acetate in hexane as gradient) yielding 8-bromo-3-(methylsulfonyl)quinoline (A15, 0.50 g, 1.747 mmol, Yield: 44.41%).

¹H NMR (DMSO-d₆, 400 MHz): δ ppm, 9.41 (d, J=2.4 Hz, 1H), 9.13 (d, J=2.4 Hz, 1H), 8.39 (dd, J=7.6, 1.2 Hz, 1H), 8.33 (dd, J=8.0, 1.2 Hz, 1H), 7.72 (t, J=8.0 Hz, 1H), 3.44 (s, 3H).

LCMS (Method A): 1.807 min, 95.24%, 254.0 nm, MS: ES+286.01 (M), 288.01 (M+2)

### Step-2:

To a stirred solution of 8-bromo-3-(methylsulfonyl)quinoline (A15, 0.3 g, 1.048 mmol, 1.0 eq), 4,4,5,5-tetramethyl-2-(4-(trifluoromethyl)cyclohex-1-en-1-yl)-1,3,2-dioxaborolane (A2, 0.289 g, 1.048 mmol, 1.0 eq) and K₃PO₄ (0.666 g, 3.144 mmol, 3.0 eq) in dioxane (15 mL) was added Pd(PPh₃)₄ (0.242 g, 0.209 mmol, 0.2 eq) under N₂. The resulting mixture was stirred at 100° C. for 2 h. The reaction was monitored by TLC (using EA:Hexane; 1.0:1.0 as mobile phase) which confirmed that the reaction got completed after 2 h of stirring at 100° C. The resulting reaction mixture was diluted with water (100 mL) and extracted with EtOAc (150 mL). The combined organic layers were dried over Na₂SO₄ and concentrated under reduced pressure to afford 0.35 g crude. The obtained crude material was purified by flash column chromatography using silica gel (230-400 mesh) as stationary phase (15% Ethyl acetate in hexane as gradient) yielding 3-(methylsulfonyl)-8-(4-(trifluoromethyl)cyclohex-1-en-1-yl)quinoline (Compound 16, 0.113 g, 0.317 mmol, Yield: 30.37%).

¹H NMR (DMSO-d₆, 400 MHz): δ ppm, 9.29 (d, J=2.4 Hz, 1H), 9.02 (d, J=2.4 Hz, 1H), 8.20 (dd, J=7.6, 2.0 Hz, 1H), 7.79-7.73 (m, 2H), 5.88 (br s, 1H), 3.40 (s, 3H), 2.79-2.76 (m, 1H), 2.76-2.70 (m, 1H), 2.29-2.26 (m, 1H), 2.11-2.08 (m, 1H), 1.75-1.65 (m, 1H). Note: 2H merged with DMSO solvent which is clearly visible in MeOD NMR.

¹H NMR (MeOD, 400 MHz): δ ppm, 9.28 (d, J=2.4 Hz, 1H), 8.96 (d, J=2.4 Hz, 1H), 8.08 (dd, J=8.0, 1.6 Hz, 1H), 7.80 (dd, J=7.2, 1.6 Hz, 1H), 7.76-7.72 (m, 1H), 5.89 (br s, 1H), 3.30 (s, 3H), 2.83-2.78 (m, 1H), 2.72-2.68 (m, 1H), 2.68-2.61 (m, 1H), 2.57-2.52 (m, 1H), 2.41-2.34 (m, 1H), 2.20-2.15 (m, 1H), 1.90-1.80 (m, 1H).

LCMS (Method A): 2.560 min, 95.70%, 254.0 nm, MS: ES+356.02 (M+1)

HPLC (Method A): 9.069 min, 95.08%, 254.0 nm

### Example 16—Synthesis of N-methyl-8-(4-(trifluoromethyl)cyclohex-1-en-1-yl)quinoline-3-sulfonamide (Compound 17)

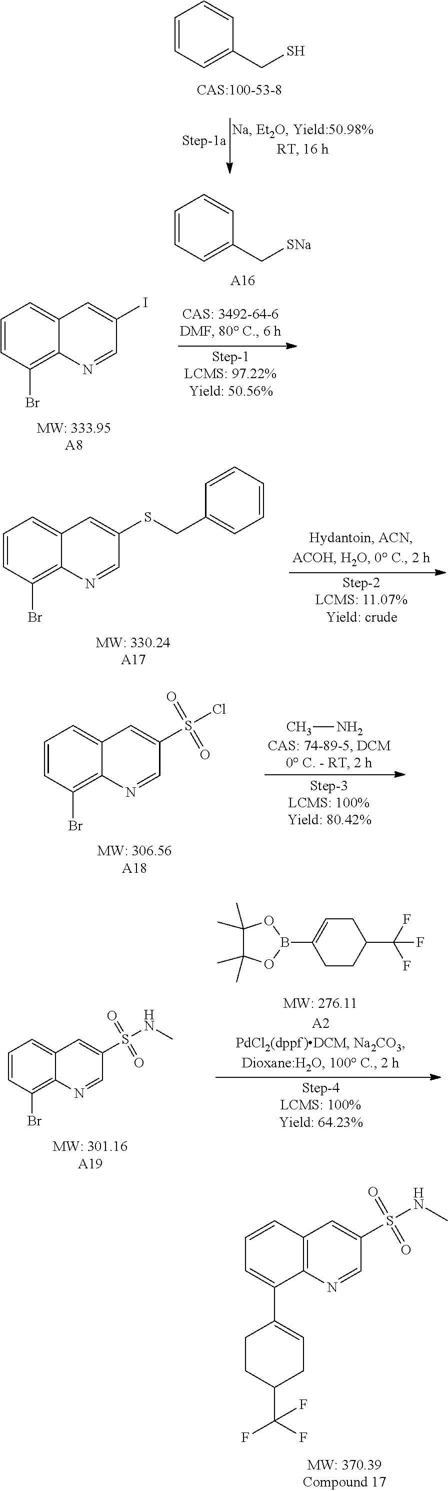

### Step-1a:

To a stirred solution of phenylmethanethiol CAS: 100-53-8 (10.0 g, 80.645 mmol, 1.0 eq) in Diethyl Ether (100 mL) was added Na Metal (0.927 g, 40.257 mmol, 0.5 eq). The resulting mixture was stirred at RT for 16 h. The reaction was monitored by TLC (using EtOAc:Hexane; 3.0:7.0 as mobile phase) which confirmed that the reaction got completed after 16 h. The resulting reaction mixture was filtered using Buchner funnel, solid material was washed with diethyl ether and dried under vacuum yielding sodium phenylmethanethiolate as white solid (A16, 6.0 g, 41.04 mmol, Yield: 50.98%).

¹H NMR (DMSO-d⁶, 400 MHz): δ ppm, 7.25 (d, J=9.2 Hz, 2H), 7.16-7.11 (m, 2H), 7.01-6.98 (m, 1H), 3.50 (br s, 2H).

### Step-1:

To a solution of 8-bromo-3-iodoquinoline (A8, 3.0 g, 8.983 mmol, 1.0 eq), in DMF (25 mL) was added sodium phenylmethanethiolate (A16, 1.83 g, 12.576 mmol, 1.4 eq) under Nitrogen. The resulting mixture was stirred for 6 h at 80° C. The reaction was monitored on TLC (using EA:Hexane; 1.0:9.0 as mobile phase) which confirmed that the reaction got completed after 6 h. The resulting reaction mixture was quenched with ice-cold water (50 mL) and extracted with EtOAc (3×50 mL). The combined organic layer were dried over Na₂SO₄ and concentrated under reduced pressure to afford 2.0 g crude. The obtained crude material was purified by flash column chromatography using silica (230-400 mesh) as stationary phase (30% Ethyl acetate in hexane as gradient) yielding 3-(benzylthio)-8-bromoquinoline (A17, 1.5 g, 4.54 mmol, Yield: 50.56%).

¹H NMR (DMSO-d6, 400 MHz): δ ppm, 8.91 (d, J=2.4 Hz, 1H), 8.39 (d, J=2.4 Hz, 1H), 8.07 (dd, J=7.6, 1.2 Hz, 1H), 7.92 (dd, J=8.0, 1.2 Hz, 1H), 7.51 (t, J=7.6 Hz, 1H), 7.41 (d, J=7.2 Hz, 2H), 7.32-7.28 (m, 2H), 7.25-7.21 (m, 1H), 4.45 (s, 2H).

LCMS (Method-A): 2.780 min, 97.22%, 254.0 nm, MS: ES+332.01 (M+2)

### Step-2:

To a stirred solution of 3-(benzylthio)-8-bromoquinoline (A17, 1.5 g, 4.542 mmol, 1.0 eq) in Acetonitrile, Acetic acid and water was added CAS: 118-52-5 (1.91 g, 9.084 mmol, 2 eq) at 0° C. and the resulting mixture was stirred at 0° C. for 2 h. The reaction was monitored by TLC (using EtOAc:Hexane; 3.0:7.0 as mobile phase) which confirmed that the reaction got completed after 2 h of stirring at 0° C. The resulting reaction mixture was concentrated to dryness then diluted with DCM and cooled to 0° C., was added 5% aq. NaHCO₃ and stirred reaction mixture for 15 minutes at 0° C., Organic layer washed with brine solution (50 ml). The combined organic layers were dried over Na₂SO₄ and concentrated under reduced pressure to afford 1.5 g crude 8-bromoquinoline-3-sulfonyl chloride (A18, 1.5 g, 4.893 mmol, Yield: Crude). Note: The crude obtained used as such for next step without further purification.

LCMS (Method A): 2.455 min, 11.07%, 254.0 nm, MS: ES+, 308.0 (M+2)

### Step-3:

To a solution of 8-bromoquinoline-3-sulfonyl chloride (A18, 1.5 g, 4.893 mmol, 1.0 eq) in DCM (20 mL) was added CAS: 74-89-5 (4.89 mL, 9.786 mmol, 2.0 eq) at 0° C. The resulting mixture was stirred for 2 h at room temperature. The reaction was monitored on TLC (using EtOAc:Hexane; 3.0:7.0 as mobile phase) which confirmed that the reaction got completed after 2 h. The resulting reaction mixture was quenched with ice-cold water (50 mL) and extracted with DCM (3×50 mL). Combined organic layers were dried over Na₂SO₄ and concentrated under reduced pressure to afford 1.4 g crude. The obtained crude material was purified by flash column chromatography using silica gel (230-400 mesh) as stationary phase (40% Ethyl acetate in hexane as gradient) yielding 8-bromo-N-methylquinoline-3-sulfonamide (A19, 1.1 g, 3.652 mmol, Yield: 80.42% (yield over 2 steps)).

¹H NMR (DMSO-d⁶, 400 MHz): δ ppm, 9.26 (d, J=2.4 Hz, 1H), 8.96 (d, J=2.0 Hz, 1H), 8.35-8.30 (m, 2H), 7.88 (q, J=4.8 Hz, 1H), 7.69 (t, J=8.0 Hz, 1H), 2.49 (s, 3H).

LCMS (Method A): 1.859 min, 100%, 254.0 nm, MS: ES+302.96 (M+2)

### Step-4:

To a stirred solution of 8-bromo-N-methylquinoline-3-sulfonamide (A19, 0.1 g, 0.332 mmol, 1.0 eq), 4,4,5,5-tetramethyl-2-(4-(trifluoromethyl)cyclohex-1-en-1-yl)-1,3,2-dioxaborolane (A2, 0.11 g, 0.398 mmol, 1.2 eq) and Na₂CO₃ (0.088 g, 0.83 mmol, 2.5 eq) in dioxane (2 mL) and water (0.4 mL) was added Pd(dppf)Cl₂·DCM (0.013 g, 0.016 mmol, 0.05 eq) under N₂. The resulting mixture was stirred at 100° C. for 2 h. The reaction was monitored on TLC (using EA:Hexane; 1.0:1.0 as mobile phase) which confirmed that the reaction got completed after 2 h of stirring at 100° C. The resulting reaction mixture was diluted with water (10 mL) and extracted with EtOAc (3×20 mL). The combined organic layers were dried over Na₂SO₄ and concentrated under reduced pressure to afford 0.120 g crude. The obtained crude material was purified by flash column chromatography using silica (230-400 mesh) as stationary phase (30% EtOAc in Hexane) N-methyl-8-(4-(trifluoromethyl)cyclohex-1-en-1-yl)quinoline-3-sulfonamide (Compound 17, 0.079 g, 0.213 mmol, Yield: 64.23%).

¹H NMR (DMSO-d⁶, 400 MHz): δ ppm, 9.13 (d, J=2.4 Hz, 1H), 8.85 (d, J=2.4 Hz, 1H), 8.17 (dd, J=6.8, 2.8 Hz, 1H), 7.74-7.70 (m, 3H), 5.87 (br s, 1H), 2.80-2.66 (m, 4H), 2.49 (s, 3H), 2.32-2.25 (m, 1H), 2.10-2.07 (m, 1H), 1.74-1.67 (m, 1H).

¹H NMR (MeOD, 400 MHz): δ ppm, 9.17 (d, J=2.4 Hz, 1H), 8.80 (d, J=2.4 Hz, 1H), 8.04 (dd, J=8.0, 1.6 Hz, 1H), 7.77-7.69 (m, 2H), 5.88 (br s, 1H), 2.83-2.78 (m, 1H), 2.71-2.66 (m, 2H), 2.62 (s, 3H), 2.57-2.51 (m, 1H), 2.40-2.34 (m, 1H), 2.19-2.16 (m, 1H), 1.91-1.89 (m, 1H).

Note: —SO₂NH proton exchanged in MeOD.

LCMS (Method A): 2.599 min, 100%, 254.0 nm, MS: ES+371.12 (M+1)

HPLC (Method A): 9.102 min, 100%, 254.0 nm

### Example 17—Synthesis of N-(1-(oxazol-2-yl)ethyl)-8-(4-(trifluoromethyl)cyclohex-1-en-1-yl)quinoline-3-carboxamide (Compound 18)

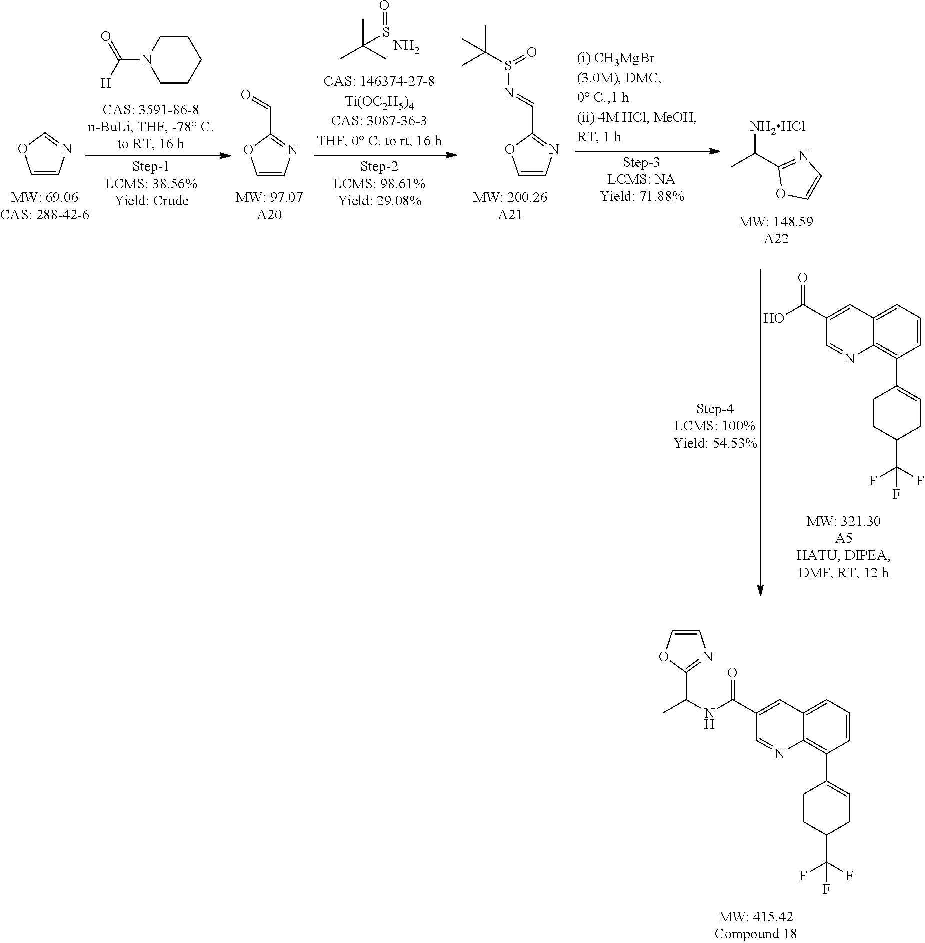

### Step-1:

To a stirred solution of oxazole CAS: 288-42-6 (0.2 g, 2.896 mmol, 1.0 eq), in THF (4 mL) was added n-BuLi (1.81 mL, 2.896 mmol, 1.5 M in Hexane, 1.0 eq) at −78° C. and continue stirred for 1 h at −78° C., then CAS: 3591-86-8 (0.327 g, 2.896 mmol, 1.0 eq) in THF (1 mL) was added under N₂. The resulting mixture was stirred at RT for 16 h. The reaction was monitored by TLC (using EA:Hexane; 1:1 as mobile phase) which confirmed that the reaction got completed after 16 h of stirring at RT. The resulting reaction mixture was quenched with 1N HCl (10 mL) and extracted with DCM (3×10 mL). The combined organic layers were dried over Na₂SO₄ and concentrated under reduced pressure to afford crude oxazole-2-carbaldehyde (A20, 0.25 g, 2.575 mmol, Yield: Crude). The obtained crude material was directly used for next step without further purification.

¹H NMR (DMSO-d6, 400 MHz): δ ppm, 9.72 (s, 1H), 7.95 (s, 1H), 7.66 (s, 1H).

LCMS (Method A): 0.196 min, 38.56%, 210.0 nm, MS: ES+, 97.9 (M+1)

### Step-2:

To a solution of oxazole-2-carbaldehyde (A20, 0.25 g, 2.57 mmol, 1.0 eq), in THF (3 mL) CAS: 146374-27-8 (0.374 g, 3.09 mmol, 1.2 eq) and CAS: 3087-36-3 (1.17 g, 5.15 mmol, 2.0 eq) were added. The resulting mixture was stirred for 16 h at room temperature. The reaction was monitored on TLC (using EtOAc:Hexane; 1:1 as mobile phase) which confirmed that the reaction got completed after 16 h. The resulting reaction mixture was quenched with brine (50 mL) and extracted with EtOAc (3×50 mL). The combined organic layers were dried over Na₂SO₄ and concentrated under reduced pressure to afford 0.3 g crude. The obtained crude material was purified by flash column chromatography using silica gel (230-400 mesh) as stationary phase (40% Ethyl acetate in hexane as gradient) yielding (E)-2-methyl-N-(oxazol-2-ylmethylene) propane-2-sulfinamide (A21, 0.15 g, 2.496 mmol, Yield: 25.87% (Yield over 2 steps)).

¹H NMR (DMSO-d6, 400 MHz): δ ppm, 8.45 (s, 1H), 8.29 (s, 1H), 7.63 (s, 1H), 1.19 (s, 9H).

LCMS (Method A): 1.551 min, 98.61%, 254.0 nm, MS: ES+201.10 (M+1)

### Step-3:

To a stirred solution of (E)-2-Methyl-N-(oxazol-2-ylmethylene) propane-2-sulfinamide (A21, 0.150 g, 0.749 mmol, 1.0 eq), in DCM (3 mL) was added CH₃MgBr (0.27 mL, 0.823 mmol, 1.1 eq) under N₂ at 0° C. The resulting mixture was stirred at 0° C. for 1 h. The reaction was monitored on TLC (using EtOAc:Hexane; 1.0:1.0 as mobile phase) which confirmed that the reaction got completed after 16 h of stirring at 0° C. The resulting reaction mixture was quenched with NH₄Cl solution (5 mL) and extracted with DCM (3×10 mL). Then crude material was dissolved in MeOH (5 mL) was added 4 M HCl in Dioxane (0.347 mL, 1.388 mmol, 2.0 eq) and stirred reaction mixture for 1 h. The reaction was monitored on TLC (using EtOAc:Hexane; 4.0:1.0 as mobile phase) which confirmed that the reaction got completed after 1 h. Reaction mixture concentrated under reduced pressure to afford 0.15 g crude. The obtained crude material was purified by trituration yielding 1-(oxazol-2-yl)ethan-1-amine (A22, 0.08 g, 0.713 mmol, Yield: 71.88%)

¹H NMR (DMSO-d₆, 400 MHz): δ ppm, 8.81 (s, 2H), 8.24 (d, J=0.8 Hz, 1H), 7.32 (d, J=0.8, 1H), 4.68-4.65 (m, 1H), 1.56 (d, J=6.8 Hz, 3H). (MeOH traces observed).

### Step-4:

To a solution of 8-(4-(trifluoromethyl)cyclohex-1-en-1-yl)quinoline-3-carboxylic acid (A5, 0.2 g, 0.622 mmol, 1.0 eq), HATU (0.35 g, 0.933 mmol, 1.5 eq) and DIPEA (0.32 mL, 1.867 mmol, 3.0 eq) in DMF (5 mL) at 0° C. under nitrogen atmosphere stirred for 20 min, was added 1-(oxazol-2-yl)ethan-1-amine (A22, 0.139 g, 0.933 mmol, 1.5 eq) under Nitrogen. The resulting mixture was stirred for 12 h at room temperature. The reaction was monitored on TLC (using EtOAc:Hexane; 7:3 as mobile phase) which confirmed that the reaction got completed after 12 h. The resulting reaction mixture was quenched with ice-cold water (10 mL) and extracted with EtOAc (3×15 mL). The combined organic layers were dried over Na₂SO₄ and concentrated under reduced pressure to afford 0.3 g crude. The obtained crude material was purified by flash column chromatography using silica (230-400 mess) as stationary phase (60% Ethyl acetate in hexane as gradient) yielding N-(1-(oxazol-2-yl)ethyl)-8-(4-(trifluoromethyl)cyclohex-1-en-1-yl)quinoline-3-carboxamide (Compound 18, 0.141 g, 0.333 mmol, Yield: 54.53%).

¹H NMR (DMSO-d6, 400 MHz): δ ppm, 9.35 (d, J=7.6 Hz, 1H), 9.27 (d, J=2.0 Hz, 1H), 8.85 (d, J=2.0 Hz, 1H), 8.08 (s, 1H), 8.02-8.00 (m, 1H), 7.66-7.62 (m, 2H), 7.19 (s, 1H), 5.87 (br s, 1H), 5.40-5.36 (m, 1H), 2.80-2.67 (m, 3H), 2.32-2.28 (m, 1H), 2.09-2.07 (m, 1H), 1.71-1.65 (m, 1H), 1.61 (d, J=7.6 Hz, 3H). Note: CF₃—CH Proton merged with DMSO Solvent peak which is clearly visible in MeOD NMR.

¹H NMR (MeOD, 400 MHz): δ ppm, 9.27 (d, J=2.4 Hz, 1H), 8.83 (d, J=2.4 Hz, 1H), 7.97 (dd, J=8.0, 2.0 Hz, 1H), 7.92 (d, J=0.4 Hz, 1H), 7.7-7.63 (m, 2H), 7.18 (d, J=0.4 Hz, 1H), 5.87 (br s, 1H), 5.52-5.46 (m, 1H), 2.75-2.68 (m, 1H), 2.68-2.65 (m, 2H), 2.56-2.51 (m, 1H), 2.40-2.37 (m, 1H), 2.18-2.14 (m, 1H), 1.92-1.83 (m, 1H), 1.73 (d, J=6.8 Hz, 3H). Note: —CONH proton exchanged with MeOD.

LCMS (Method A): 2.466 min, 100%, 254.0 nm, MS: ES+416.18 (M+1) HPLC (Method A): 8.717 min, 99.37%, 254.0 nm

### Example 18—Chiral separation of N-(1-(oxazol-2-yl)ethyl)-8-(4-(trifluoromethyl)cyclohex-1-en-1-yl)quinoline-3-carboxamide (Compounds 19-22)

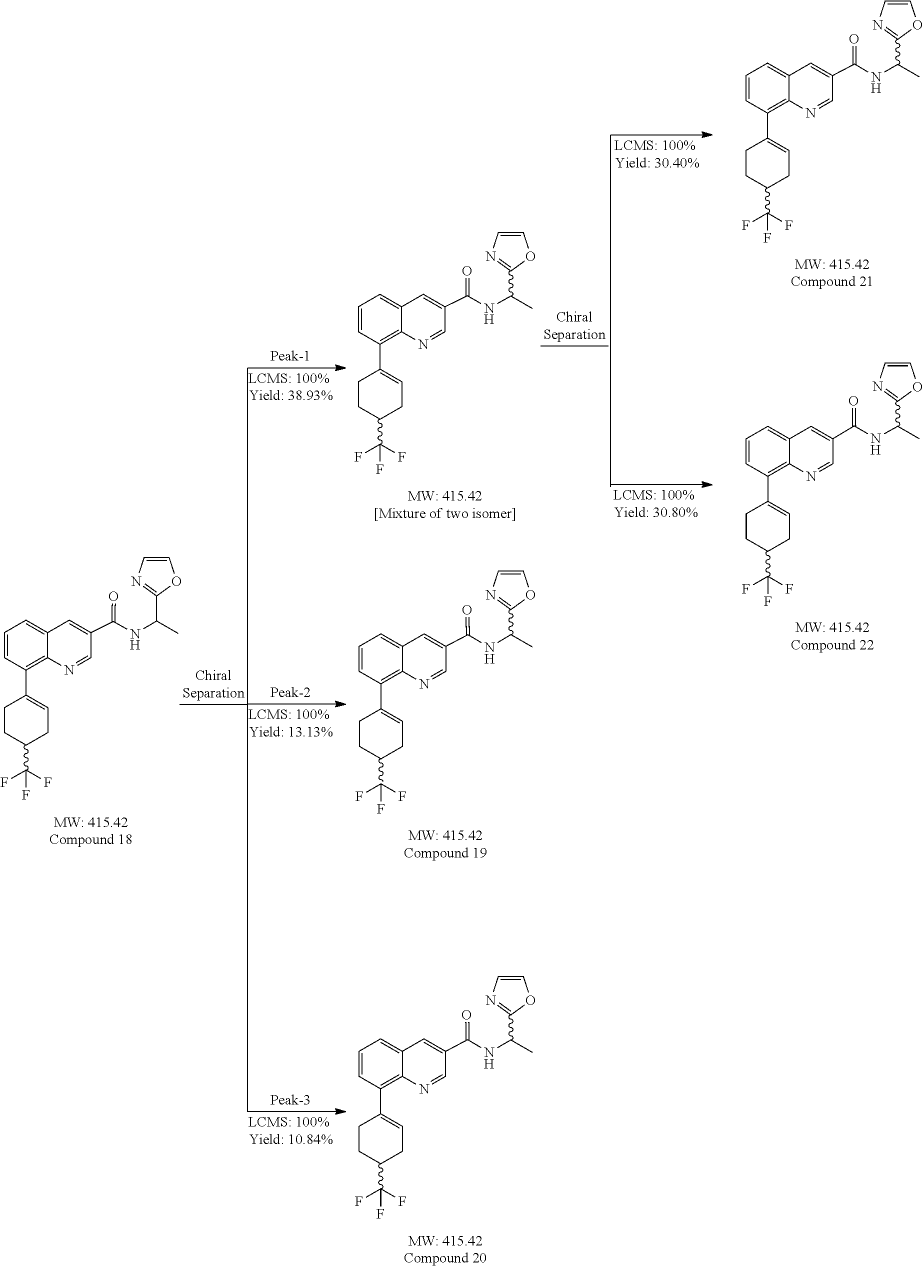 [SMILES: CC(NC(=O)c1cnc2c(C3=CCC(C(F)(F)F)CC3)cccc2c1)c1ncco1 ; CC(NC(=O)c1cnc2c(C3=CCC(C(F)(F)F)CC3)cccc2c1)c1ncco1 ; CC(NC(=O)c1cnc2c(C3=CCC(C(F)(F)F)CC3)cccc2c1)c1ncco1 ; CC(NC(=O)c1cnc2c(C3=CCC(C(F)(F)F)CC3)cccc2c1)c1ncco1 ; CC(NC(=O)c1cnc2c(C3=CCC(C(F)(F)F)CC3)cccc2c1)c1ncco1 ; CC(NC(=O)c1cnc2c(C3=CCC(C(F)(F)F)CC3)cccc2c1)c1ncco1]

### Procedure:

Compound 18 (0.131 g) racemic was subjected to chiral SFC purification on (CHIRALPAK IG 250×50 mm 5 μm) column where 3 peaks were separated: Peak 1 (0.051 g, Yield=38.93%), Peak 2 (Compound 19, 0.0172 g Yield=13.13%), and Peak 3 (Compound 20 0.0142 g Yield=10.84%)

COLUMN ID: CHIRALPAK IG 250×50 mm 5 μm

MOBILE PHASE A: LIQ. CO₂

MOBILE PHASE B: 0.1% M·NH₃ IN MEOH-ACN (50-50)

FLOW RATE (ML/MIN): 170

INSTRUMENT ID: WATERS SFC 350 WITH 2489 UV Detector

METHOD: TIME: FLOW: % A: % B (0.01:170:55:45), (17:170:55:45)

### Compound 19:

¹H NMR (DMSO-d6, 400 MHz): δ ppm, 9.35 (d, J=7.6 Hz, 1H), 9.27 (d, J=2.4 Hz, 1H), 8.86 (d, J=2.4 Hz, 1H), 8.08 (d, J=0.8 Hz, 1H), 8.03-8.00 (m, 1H), 7.67-7.63 (m, 2H), 7.19 (d, J=0.4 Hz, 1H), 5.88 (br s, 1H), 5.40-5.36 (m, 1H), 2.72-2.67 (m, 4H), 2.33-2.29 (m, 1H), 2.10-2.07 (m, 1H), 1.71-1.67 (m, 1H), 1.61 (d, J=6.8 Hz, 3H) (Note: -one CF₃—CH proton merge with DMSO solvent peak and clearly visible in MeOD NMR)

¹H NMR (MeOD, 400 MHz): δ ppm, 9.28 (d, J=2.4 Hz, 1H), 8.83 (d, J=2.0 Hz, 1H), 7.98 (dd, J=7.6, 1.6 Hz, 1H), 7.92 (d, J=0.8 Hz, 1H), 7.71-7.64 (m, 2H), 7.18 (s, 1H), 5.87 (br s, 1H), 5.52-5.47 (m, 1H), 2.79-2.66 (m, 4H), 2.56-2.37 (m, 1H), 2.18-2.15 (m, 1H), 1.93-1.82 (m, 1H), 1.73 (d, J=7.2 Hz, 3H). (Note: —NH proton might exchange with deuterium from MeOD)

LCMS (Method A): 2.465 min, 100%, 254.0 nm, MS: ES+416.2 (M+1)

HPLC (Method A): 8.775 min, 100%, 254.0 nm

### Compound 20:

¹H NMR (DMSO-d6, 400 MHz): δ ppm, 9.35 (d, J=7.6 Hz, 1H), 9.27 (d, J=2.4 Hz, 1H), 8.86 (d, J=2.4 Hz, 1H), 8.08 (d, J=0.8 Hz, 1H), 8.03-8.00 (m, 1H), 7.67-7.63 (m, 2H), 7.19 (d, J=0.4 Hz, 1H), 5.88 (br s, 1H), 5.40-5.36 (m, 1H), 2.72-2.67 (m, 4H), 2.33-2.29 (m, 1H), 2.10-2.07 (m, 1H), 1.71-1.67 (m, 1H), 1.61 (d, J=6.8 Hz, 3H) (Note: -one CF₃—CH proton merge with DMSO solvent peak and clearly visible in MeOD NMR)

¹H NMR (MeOD, 400 MHz): δ ppm, 9.28 (d, J=2.4 Hz, 1H), 8.83 (d, J=2.0 Hz, 1H), 7.98 (dd, J=7.6, 1.6 Hz, 1H), 7.92 (d, J=0.8 Hz, 1H), 7.71-7.64 (m, 2H), 7.18 (s, 1H), 5.87 (br s, 1H), 5.52-5.47 (m, 1H), 2.79-2.66 (m, 4H), 2.56-2.37 (m, 1H), 2.18-2.15 (m, 1H), 1.93-1.82 (m, 1H), 1.73 (d, J=7.2 Hz, 3H) (Note: —NH proton might exchange with deuterium from MeOD)

LCMS (Method A): 2.466 min, 100%, 254.0 nm, MS: ES+416.2 (M+1)

HPLC (Method A): 8.772 min, 99.71%, 254.0 nm

Chiral Separation of Compound 21 & Compound 22:

Procedure: Peak 1 (0.051 g) was further subjected for chiral SFC purification on (CHIRALPAK IG 250×50 mm 5 um) column in order to obtain Peak 1 Isomer 1 (Compound 21, 0.0131 g, Yield=26.20%), and Peak 1 Isomer 2 (Compound 22, 0.0129 g, Yield=25.80%)

COLUMN ID: CHIRALPAK IG 250×50 mm 5 um

MOBILE PHASE A: LIQ. CO2

MOBILE PHASE B: 0.1% M·NH3 in IPA-MEOH (70-30)

FLOW RATE (ML/MIN): 150

INSTRUMENT ID: WATERS SFC 350 WITH 2489 UV Detector

METHOD: TIME: FLOW: % A: % B (0.01:150:70:30), (18:150:70:30)

### Compound 21:

¹H NMR (DMSO-d6, 400 MHz): δ ppm, 9.35 (d, J=7.6 Hz, 1H), 9.27 (d, J=2.4 Hz, 1H), 8.86 (d, J=2.4 Hz, 1H), 8.08 (d, J=0.8 Hz, 1H), 8.03-8.00 (m, 1H), 7.67-7.63 (m, 2H), 7.19 (d, J=0.4 Hz, 1H), 5.88 (br s, 1H), 5.40-5.36 (m, 1H), 2.72-2.67 (m, 4H), 2.33-2.29 (m, 1H), 2.10-2.07 (m, 1H), 1.71-1.67 (m, 1H), 1.61 (d, J=6.8 Hz, 3H) (Note: -one CF₃—CH proton merge with DMSO solvent peak and clearly visible in MeOD NMR)

¹H NMR (MeOD, 400 MHz): δ ppm, 9.28 (d, J=2.4 Hz, 1H), 8.83 (d, J=2.0 Hz, 1H), 7.98 (dd, J=7.6, 1.6 Hz, 1H), 7.92 (d, J=0.8 Hz, 1H), 7.71-7.64 (m, 2H), 7.18 (s, 1H), 5.87 (bs, 1H), 5.52-5.47 (m, 1H), 2.79-2.66 (m, 4H), 2.56-2.37 (m, 1H), 2.18-2.15 (m, 1H), 1.93-1.82 (m, 1H), 1.73 (d, J=7.2 Hz, 3H). (Note: —NH proton might exchange with deuterium from MeOD)

LCMS (Method A): 2.427 min, 100%, 254.0 nm, MS: ES+416.2 (M+1)

HPLC (Method A): 8.859 min, 100%, 254.0 nm

### Compound 22:

¹H NMR (DMSO-d6, 400 MHz): δ ppm, 9.35 (d, J=7.6 Hz, 1H), 9.27 (d, J=2.4 Hz, 1H), 8.86 (d, J=2.4 Hz, 1H), 8.08 (d, J=0.8 Hz, 1H), 8.03-8.00 (m, 1H), 7.67-7.63 (m, 2H), 7.19 (d, J=0.4 Hz, 1H), 5.88 (br s, 1H), 5.40-5.36 (m, 1H), 2.72-2.67 (m, 4H), 2.33-2.29 (m, 1H), 2.10-2.07 (m, 1H), 1.71-1.67 (m, 1H), 1.61 (d, J=6.8 Hz, 3H) (Note: -one CF₃—CH proton merge with DMSO solvent peak and clearly visible in MeOD NMR)

¹H NMR (MeOD, 400 MHz): δ ppm, 9.28 (d, J=2.4 Hz, 1H), 8.83 (d, J=2.0 Hz, 1H), 7.98 (dd, J=7.6, 1.6 Hz, 1H), 7.92 (d, J=0.8 Hz, 1H), 7.71-7.64 (m, 2H), 7.18 (s, 1H), 5.87 (br s, 1H), 5.52-5.47 (m, 1H), 2.79-2.66 (m, 4H), 2.56-2.37 (m, 1H), 2.18-2.15 (m, 1H), 1.93-1.82 (m, 1H), 1.73 (d, J=7.2 Hz, 3H). (Note: —NH proton might exchange with deuterium from MeOD)

LCMS (Method A): 2.420 min, 100%, 254.0 nm, MS: ES+416.1 (M+1)

HPLC (Method A): 8.851 min, 100%, 254.0 nm

### Example 19—Synthesis of N-(2-(methylthio)ethyl)-8-(4-(trifluoromethyl)cyclohex-1-en-1-yl)quinoline-3-carboxamide (Compound 23)

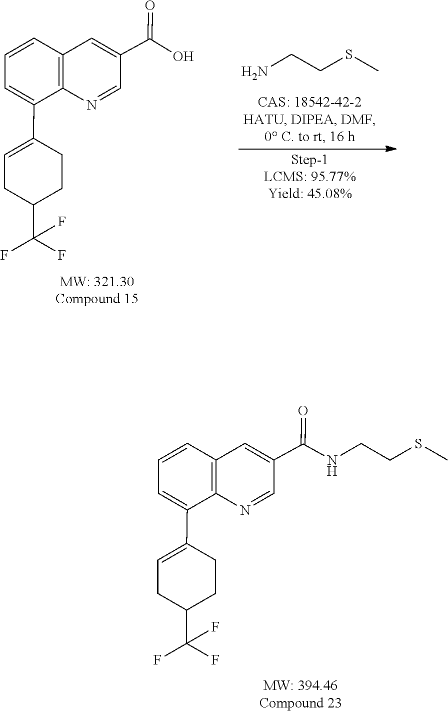

### Step-1:

A stirred solution of 8-(4-(trifluoromethyl)cyclohex-1-en-1-yl)quinoline-3-carboxylic acid (Compound 15, 0.1 g, 0.31 mmol, 1.0 eq), in DMF (1 mL) was prepared in 10 mL glass vial at room temperature. To this reaction solution, DIPEA (0.16 mL, 0.93 mmol, 3.0 eq) and HATU (0.17 g, 0.46 mmol, 1.5 eq) was added at 0° C. under nitrogen atmosphere. After 30 min of stirring at 0° C., (CAS: 18542-42-2) 2-(methylthio) ethan-1-amine (0.028 g, 0.31 mmol, 1.0 eq) was added at same temperature. Then the resulting reaction mixture was stirred from 0° C. to RT for 16 h. The reaction was monitored by TLC (using EtOAc:Hexane; 4.0:6.0 as mobile phase) which confirmed that the reaction got completed after 16 h of stirring at RT. The resulting reaction mixture was diluted with water (5 mL) and extracted with EtOAc (3×5 mL). The combined organic layers were dried over Na₂SO₄, filtered and concentrated under reduced pressure to afford 0.11 g crude. The obtained crude material was purified by flash column chromatography using silica (230-400 mesh size) as a stationary phase (elution gradient 20% EtOAc in Hexane) yielding as a N-(2-(methylthio)ethyl)-8-(4-(trifluoromethyl)cyclohex-1-en-1-yl)quinoline-3-carboxamide (Compound 23, 0.055 g, 0.13 mmol, Yield: 45.08%)

¹H NMR (DMSO-d₆, 400 MHz): δ ppm, 9.25 (d, J=2.4 Hz, 1H), 8.96 (t, J=5.6 Hz, 1H), 8.79 (d, J=2.0 Hz, 1H), 8.02-7.99 (m, 1H), 7.65-7.61 (m, 2H), 5.86 (br s, 1H), 3.53 (q, J=6.4 Hz, 2H), 2.80-2.77 (m, 1H), 2.72-2.67 (m, 4H), 2.32-2.20 (m, 1H), 2.13-2.06 (m, 4H), 1.74-1.67 (m, 1H). (Note: -one CF₃—CH proton merge with DMSO solvent peak and clearly visible in MeOD NMR)

¹H NMR (MeOD, 400 MHz): δ ppm, 9.24 (d, J=2.4 Hz, 1H), 8.75 (d, J=2.0 Hz, 1H), 7.96 (d, J=8.0 Hz, 1H), 7.69-7.62 (m, 2H), 5.86 (br s, 1H), 3.68 (t, J=6.8 Hz, 2H), 2.80 (t, J=7.2 Hz, 3H), 2.68-2.60 (m, 2H), 2.55-2.50 (m, 1H), 2.40-2.33 (m, 1H), 2.19-2.15 (m, 4H), 1.92-1.81 (m, 1H) (Note: —NH proton might exchange with deuterium from MeOD)

LCMS (Method A): 2.614 min, 95.77%, 254 nm, MS: ES+395.12 (M+1)

HPLC (Method B): 9.433 min, 95.17%, 254 nm

### Example 20—Synthesis of N-(2-(methylsulfinyl)ethyl)-8-(4-(trifluoromethyl)cyclohex-1-en-1-yl)quinoline-3-carboxamide (Compound 24)

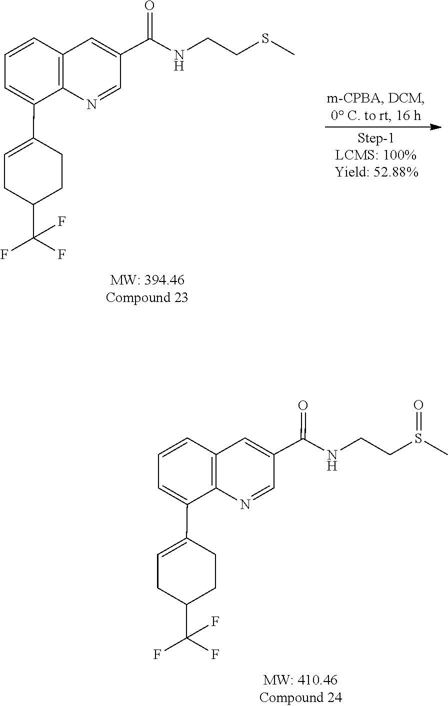 [SMILES: CSCCNC(=O)c1cnc2c(C3=CCC(C(F)(F)F)CC3)cccc2c1 ; CS(=O)CCNC(=O)c1cnc2c(C3=CCC(C(F)(F)F)CC3)cccc2c1]

### Step-1:

A stirred solution of N-(2-(methylthio)ethyl)-8-(4-(trifluoromethyl)cyclohex-1-en-1-yl)quinoline-3-carboxamide (Compound 23, 0.1 g, 0.25 mmol, 1.0 eq), in DCM (1 mL) was prepared in 10 mL glass vial at room temperature. To this resulting mixture m-CPBA (60% assay) (0.026 g, 0.25 mmol, 1.0 eq) in DCM (0.5 mL) was added dropwise at 0° C. under nitrogen atmosphere. Then the resulting reaction mixture was stirred from 0° C. to RT for 16 h. The reaction was monitored by TLC (using MeOH:DCM; 0.5:9.5 as mobile phase) which confirmed that the reaction got completed after 16 h of stirring at RT. The resulting reaction mixture was diluted with water (5 mL), basified with solution of sat NaHCO₃ (5 mL) and extracted with DCM (3×5 mL). The combined organic layers were dried over Na₂SO₄, filtered and concentrated under reduced pressure to afford 0.11 g crude. The obtained crude material was purified by flash column chromatography using silica (230-400 mesh) as a stationary phase (elution gradient 3% MeOH in DCM) yielding as a N-(2-(methylsulfinyl)ethyl)-8-(4-(trifluoromethyl)cyclohex-1-en-1-yl)quinoline-3-carboxamide (Compound 24, 0.055 g, 0.13 mmol, Yield: 52.88%)

1H NMR (DMSO-d6, 400 MHz): δ ppm, 9.24 (d, J=2.0 Hz, 1H), 9.13 (t, J=5.2 Hz, 1H), 8.80 (d, J=2.0 Hz, 1H), 8.02-8.00 (m, 1H), 7.65-7.62 (m, 2H), 5.87 (br s, 1H), 3.77-3.64 (m, 2H), 3.15-3.08 (m, 1H), 2.97-2.91 (m, 1H), 2.79-2.71 (m, 2H), 2.62 (s, 3H), 2.32-2.25 (m, 1H), 2.10-2.07 (m, 1H), 1.74-1.67 (m, 1H). (Note: -one CF₃—CH proton merge with DMSO solvent peak and clearly visible in MeOD NMR).

¹H NMR (MeOD, 400 MHz): δ ppm, 9.25 (d, J=2.0 Hz, 1H), 8.76 (d, J=2.0 Hz, 1H), 7.96 (t, J=6.4 Hz, 1H), 7.69-7.63 (m, 2H), 5.86 (br s, 1H), 3.95-3.89 (m, 2H), 3.28-3.23 (m, 1H), 3.13-3.07 (s, 1H), 2.76 (s, 4H), 2.68-2.65 (m, 2H), 2.55-2.51 (m, 1H), 2.40-2.37 (m, 1H), 2.18-2.15 (m, 1H), 1.89-1.85 (m, 1H). (Note: —NH proton might exchange with deuterium from MeOD)

LCMS (Method A): 2.121 min, 100%, 254 nm, MS: ES+411.1 (M+1)

HPLC (Method B): 7.334 min, 99.45%, 254 nm

### Example 21—Synthesis of N-(2-(methyl sulfonyl)ethyl)-8-(4-(trifluoromethyl)cyclohex-1-en-1-yl)quinoline-3-carboxamide (Compound 25)

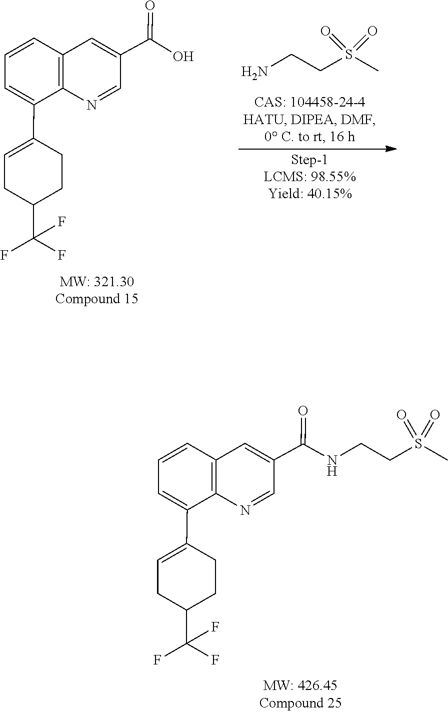

### Step-1:

A stirred solution of 8-(4-(trifluoromethyl)cyclohex-1-en-1-yl)quinoline-3-carboxylic acid (Compound 15, 0.1 g, 0.31 mmol, 1.0 eq), in DMF (1 mL) was prepared in 10 mL glass vial at room temperature. To this reaction solution, DIPEA (0.27 mL, 1.55 mmol, 5.0 eq) and HATU (0.17 g, 0.46 mmol, 1.5 eq) was added at 0° C. under nitrogen atmosphere. After 30 min of stirring at 0° C., 2-(methyl sulfonyl)ethan-1-amine (CAS: 104458-24-4) (0.049 g, 0.31 mmol, 1.0 eq) was added at same temperature. Then the resulting reaction mixture was stirred from 0° C. to RT for 16 h. The reaction was monitored by TLC (using Neat EtOAc as a mobile phase) which confirmed that the reaction got completed after 16 h of stirring at RT. The resulting reaction mixture was diluted with water (5 mL) and extracted with EtOAc (3×15 mL). The combined organic layers were washed with cold water (3×30 mL), dried over Na₂SO₄, filtered and concentrated under reduced pressure to afford 0.12 g crude. The obtained crude material was purified by trituration with diethyl ether yielding as a N-(2-(methyl sulfonyl)ethyl)-8-(4-(trifluoromethyl)cyclohex-1-en-1-yl)quinoline-3-carboxamide (Compound 25, 0.053 g, 0.12 mmol, Yield: 40.15%).

¹H NMR (DMSO-d₆, 400 MHz): δ ppm, 9.24 (d, J=2.0 Hz, 1H), 9.09 (t, J=5.2 Hz, 1H), 8.79 (d, J=1.6 Hz, 1H), 8.02-8.00 (m, 1H), 7.65-7.62 (m, 2H), 5.87 (br s, 1H), 3.77 (q, J=6.4 Hz, 2H), 3.44 (t, J=6.8 Hz, 2H), 3.07 (s, 3H), 2.80-2.77 (m, 1H), 2.71-2.67 (m, 2H), 2.46-2.43 (m, 1H), 2.32-2.28 (m, 1H), 2.10-2.07 (m, 1H), 1.71-1.67 (m, 1H) (Note: -one CF₃—CH proton merge with DMSO solvent peak and clearly visible in MeOD NMR)

¹H NMR (MeOD, 400 MHz): δ ppm, 9.24 (d, J=2.4 Hz, 1H), 8.76 (d, J=2.0 Hz, 1H), 7.97 (dd, J=7.6, 3.6 Hz, 1H), 7.69-7.63 (m, 2H), 5.86 (s, 1H), 3.95 (t, J=6.4 Hz, 2H), 3.51 (t, J=6.8 Hz, 2H), 3.09 (s, 3H), 2.78-2.74 (m, 1H), 2.68-2.64 (m, 2H), 2.55-2.51 (m, 1H), 2.40-2.37 (m, 1H), 2.18-2.15 (m, 1H), 1.89-1.85 (m, 1H). (Note: —NH proton might exchange with deuterium from MeOD)

LCMS (Method A): 2.290 min, 98.55%, 254 nm, MS: ES+427.2 (M+1)

HPLC (Method B): 8.152 min, 98.63%, 254 nm

### Example 22—Synthesis of 8-(4,4-difluorocyclohex-1-en-1-yl)-N-isopropylquinoline-3-carboxamide (Compound 26)

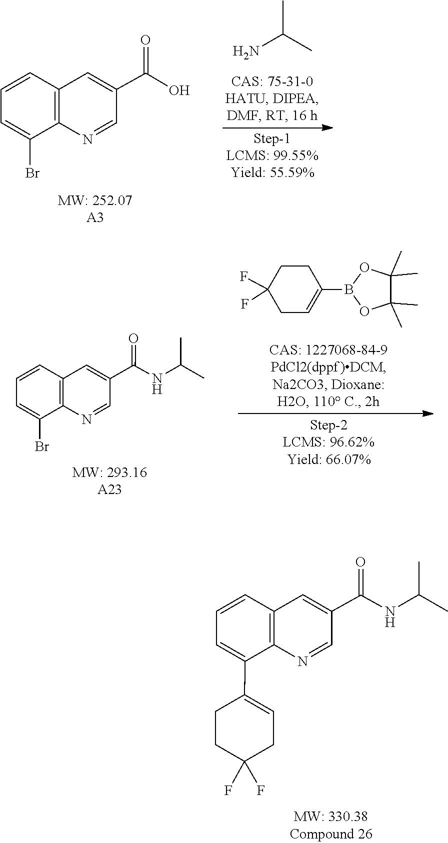

### Step-1:

A stirred solution of 8-bromoquinoline-3-carboxylic acid (A3, 0.50 g, 1.98 mmol, 1.0 eq) in DMF (5.0 mL) was prepared in 30 mL of glass vial at room temperature. To this reaction solution, DIPEA (0.76 g, 5.95 mmol, 3.0 eq) and HATU (1.12 g, 2.97 mmol, 1.5 eq) were added at 0° C. under nitrogen atmosphere. After 10 min of stirring isopropyl amine (CAS: 75-31-0) (0.11 g, 1.98 mmol, 1.0 eq) was added at same temperature. Then the resulting reaction mixture was stirred at RT for 16 h. The reaction was monitored by TLC (using EtOAc:Hexane; 1.0:1.0 as mobile phase) which confirmed that the reaction was completed after 16 h of stirring at RT. The resulting reaction mixture was extracted by EtOAc (50 mL) and H₂O (50 mL), Organic layer was dried on Na₂SO₄, filtered and concentrated under high vacuum to obtained crude product. The crude product was purified by combi-flash using 230-400 mesh size silica and product was eluting at 12% EtOAc:Hexane to yielding 8-bromo-N-isopropylquinoline-3-carboxamide (A23, 0.323 g, 1.101 mmol, Yield: 55.59%)

¹H NMR (DMSO-d₆, 400 MHz): δ ppm, 9.33 (d, J=2.0 Hz, 1H), 8.86 (d, J=2.0 Hz, 1H), 8.67 (d, J=4.8 Hz, 1H), 8.24 (d, J=6.8 Hz, 1H), 8.14 (d, J=8.0 Hz, 1H), 7.61 (t, J=8.0 Hz, 1H), 4.21-4.12 (m, 1H), 1.23 (d, J=6.4 Hz, 6H).

LCMS (Method A): 1.866 min, 99.55%, 254 nm, MS: ES+294 (M+1)

### Step-2:

A stirred solution of A23 (0.1 g, 0.34 mmol, 1 eq) in Dioxane:H₂O (3:1) was prepared in 10 mL glass vial at room temperature. To this reaction solution, CAS: 1227068-84-9 (0.08 g, 0.33 mmol, 1 eq), Na₂CO₃ (0.10 g, 1.02 mmol, 3 eq) was added at same temperature. Purge by N₂. After that Pd(dppf)Cl₂ (0.02 g, 0.03 mmol, 0.1 eq) was added and stirred at 110° C. for 2 h. Then reaction was monitored by TLC (50% EtOAc:Hexane as a mobile phase) which confirmed that the reaction got completed after 2 h of stirring at 110° C. The resulting reaction mixture was dilute with water (20 mL) and extracted with EtOAc (20 mL). The Organic layer was dried on Na₂SO₄, filtered and concentrated under high vacuum to obtained crude product. The crude product was purified by combi-flash column chromatography using 230-400 mesh size silica and product was eluted at 10% EtOAc:Hexane to yielding as 8-(4,4-difluorocyclohex-1-en-1-yl)-N-isopropylquinoline-3-carboxamide (Compound 26, 0.074 g, 0.223 mmol, Yield: 66.07%).

¹H NMR (DMSO-d₆, 400 MHz): δ ppm, 9.25 (d, J=1.6 Hz, 1H), 8.79 (d, J=1.6 Hz, 1H), 8.56 (d, J=7.2 Hz, 1H), 8.01 (t, J=5.6 Hz, 1H), 7.63 (t, J=7.2 Hz, 2H), 5.76 (br s, 1H), 4.18-4.13 (m, 1H), 2.90 (br s, 2H), 2.79 (t, J=14.8 Hz, 2H), 2.25-2.18 (m, 2H), 1.21 (d, J=6.4 Hz, 6H).

LCMS (Method A): 2.313 min, 96.62%, 254.0 nm, MS: ES+331.2 (M+1)

HPLC (Method B): 8.486 min, 99.62% at 254.0 nm.

### Example 23—Synthesis of(S)-8-(4,4-difluorocyclohex-1-en-1-yl)-N-(1-(pyridin-2-yl)ethyl)quinoline-3-carboxamide (Compound 27)

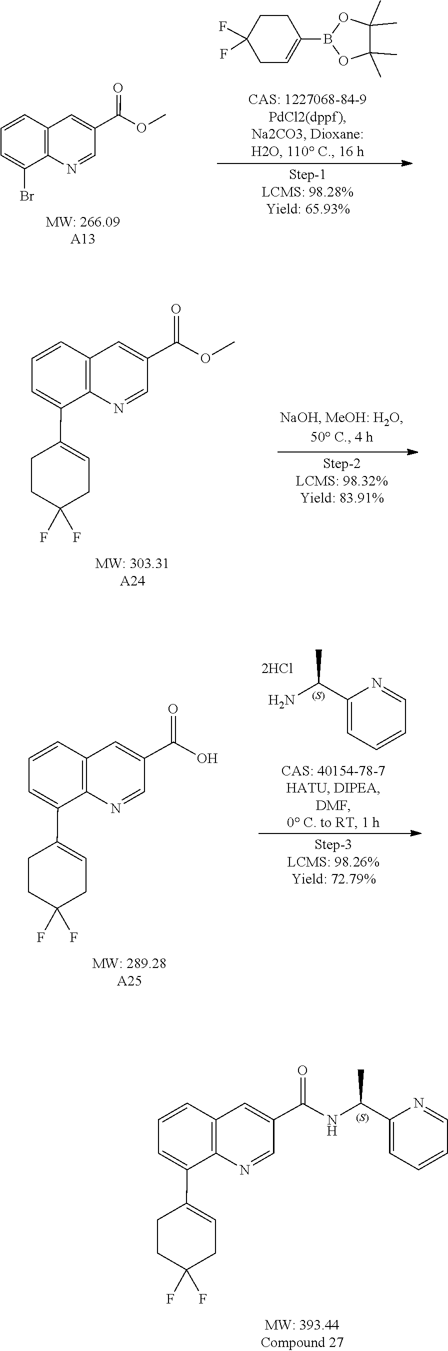

### Step-1:

A stirred solution of methyl 8-bromoquinoline-3-carboxylate (A13, 0.4 g, 1.50 mmol, 1.0 eq), 2-(4,4-difluorocyclohex-1-en-1-yl)-4,4,5,5-tetramethyl-1,3,2-dioxaborolane (CAS: 1227068-84-9) (0.36 g, 1.50 mmol, 1.0 eq) and Na₂CO₃ (0.47 g, 4.50 mmol, 3.0 eq) in Dioxane:water (9:1) was prepared in 30 mL of glass vial at room temperature under N₂ atmosphere. The reaction solution was degassed by N₂ for 15 minutes at room temperature. To this reaction solution, PdCl₂(dppf) (0.10 g, 0.15 mmol, 0.1 eq) was added at same temperature. The glass vial was sealed with cap and heated to 110° C. for 16 h. The reaction was monitored by TLC (using EtOAc:Hexane; 3.0:7.0 as mobile phase) which confirmed that the reaction got completed after 16 h of stirring at 110° C. The resulting reaction mixture was cooled to room temperature, diluted with EtOAc (10 mL), filtered it through celite bed, washed it with EtOAc (30 mL) and filtrate was concentrated under reduced pressure to afford 0.7 g crude. The obtained crude material was purified by flash column chromatography using silica (230-400 mesh) as a stationary phase (10% EtOAc in hexane) yielding as a methyl 8-(4,4-difluorocyclohex-1-en-1-yl)quinoline-3-carboxylate (A24, 0.3 g, 0.98 mmol, Yield: 65.93%)

¹H NMR (DMSO-d₆, 400 MHz): δ ppm, 9.31 (d, J=2.0 Hz, 1H), 9.02 (d, J=2.0 Hz, 1H), 8.16 (dd, J=8.0 Hz, 1.2 Hz, 1H), 7.73 (dd, J=6.8 Hz, 1.2 Hz, 1H), 7.68 (t, J=7.6 Hz, 1H), 5.76 (br s, 1H) 3.96 (s, 3H), 2.89 (br s, 2H), 2.83-2.75 (m, 2H), 2.27-2.17 (m, 2H).

LCMS (Method A): 2.613 min, 98.28%, 254 nm, MS: ES+304.1 (M+1)

### Step-2:

A stirred solution of methyl 8-(4,4-difluorocyclohex-1-en-1-yl)quinoline-3-carboxylate (A24, 0.3 g, 0.98 mmol, 1.0 eq) in MeOH:water (7:3) was prepared in 30 mL of glass vial at room temperature. To this reaction solution, NaOH (0.10 g, 2.630 mmol, 2.0 eq) was added at same temperature. The glass vial was sealed with cap and heated to 50° C. for 4 h. The reaction was monitored by TLC (using EtOAc:Hexane; 3.0:7.0 as mobile phase) which confirmed that the reaction got completed after 4 h of stirring at 110° C. The resulting reaction mixture was cooled to room temperature, diluted with MeOH (5 mL) and concentrated under reduced pressure. To this reaction mixture was added aq. solution of citric acid until pH become acidic to get precipitate. The obtain precipitates were filtered through Buckner funnel, washed with water (10 mL) and dried over reduce vacuum to yield 8-(4,4-difluorocyclohex-1-en-1-yl)quinoline-3-carboxylic acid (A25, 0.24 g, 0.82 mmol, Yield: 83.91%).

¹H NMR (DMSO-d₆, 400 MHz): δ ppm, 9.30 (d, J=2.0 Hz, 1H), 8.97 (d, J=2.0 Hz, 1H), 8.13 (dd, J=8.0 Hz, 1.2 Hz, 1H), 7.72-7.64 (m, 2H), 5.76 (br s, 1H), 2.89 (br s, 2H), 2.79 (t, J=14.4 Hz, 2H), 2.27-2.17 (m, 2H). Note: —COOH proton might exchange with DMSO moisture.

LCMS (Method A): 2.164 min, 98.32%, 254 nm, MS: ES+290.1 (M+1)

### Step-3:

A stirred solution of 8-(4,4-difluorocyclohex-1-en-1-yl)quinoline-3-carboxylic acid (A25, 0.1 g, 0.34 mmol, 1.0 eq) in DMF (1.0 mL) was prepared in 10 mL of glass vial at room temperature. To this reaction solution, DIPEA (0.30 mL, 1.72 mmol, 5.0 eq) and HATU (0.19 g, 0.51 mmol, 1.5 eq) were added at 0° C. under nitrogen atmosphere. After 30 min of stirring at 0° C., (1S)-1-(Pyridin-2-yl)ethan-1-amine hydrochloride (CAS: 40154-78-7) (0.067 g, 0.34 mmol, 1.0 eq) was added at same temperature. Then the resulting reaction mixture was stirred from 0° C. to RT for 1 h. The reaction was monitored by TLC (using neat EtOAc as a mobile phase) which confirmed that the reaction got completed after 1 h of stirring from 0° C. to RT. The resulting reaction mixture was diluted with cold water (5 mL) and extracted with EtOAc (3×5 mL). The combined organic layers were dried over Na₂SO₄, filtered and concentrated under reduced pressure to afford 0.12 g crude. The obtained crude material was purified by flash column chromatography using silica (230-400 mesh) as a stationary phase (elution gradient 30% EtOAc in Hexane) yielding as a(S)-8-(4,4-difluorocyclohex-1-en-1-yl)-N-(1-(pyridin-2-yl)ethyl)quinoline-3-carboxamide (Compound 27, 0.099 g, 0.25 mmol, Yield: 72.79%)

¹H NMR (DMSO-d₆, 400 MHz): δ ppm, 9.31 (d, J=2.4 Hz, 1H), 9.20 (d, J=8.0 Hz, 1H), 8.90 (d, J=2.4 Hz, 1H), 8.54 (d, J=4.0 Hz, 1H), 8.04 (dd, J=7.2, 2.0 Hz, 1H), 7.78 (dt, J=8.0 Hz, 2.0 Hz, 1H), 7.69-7.63 (m, 2H), 7.48 (d, J=8.0 Hz, 1H), 7.29-7.26 (m, 1H), 5.77 (br s, 1H), 5.30-5.22 (m, 1H), 2.90-2.89 (m, 2H), 2.79 (t, J=15.6 Hz, 2H), 2.26-2.19 (m, 2H), 1.55 (d, J=6.8 Hz, 3H).

¹H NMR (MeOD, 400 MHz): δ ppm, 9.29 (d, J=2.4 Hz, 1H), 8.85 (d, J=2.4 Hz, 1H), 8.55 (d, J=4.4 Hz, 1H), 8.00 (d, J=8.0 Hz, 1H), 7.87-7.83 (m, 1H), 7.70-7.63 (m, 2H), 7.54 (d, J=8.0 Hz, 1H), 7.36-7.32 (m, 1H), 5.75 (br s, 1H), 5.35 (q, J=7.2 Hz, 1H), 2.89-2.86 (m, 2H), 2.83-2.74 (m, 2H), 2.32-2.25 (m, 2H), 1.66 (d, J=7.2 Hz, 3H). (Note: —NH proton might exchange with deuterium from MeOD)

LCMS (Method A): 2.027 min, 95.17%, 210 nm, MS: ES+394.2 (M+1)

HPLC (Method B): 8.283 min, 98.25%, 254 nm

Chiral HPLC (Method C): 2.59 min, 98.10%, 240 nm

### Example 24—Synthesis of(S)-8-(4,4-difluorocyclohex-1-en-1-yl)-N-(1-hydroxypropan-2-yl)quinoline-3-carboxamide (Compound 28)

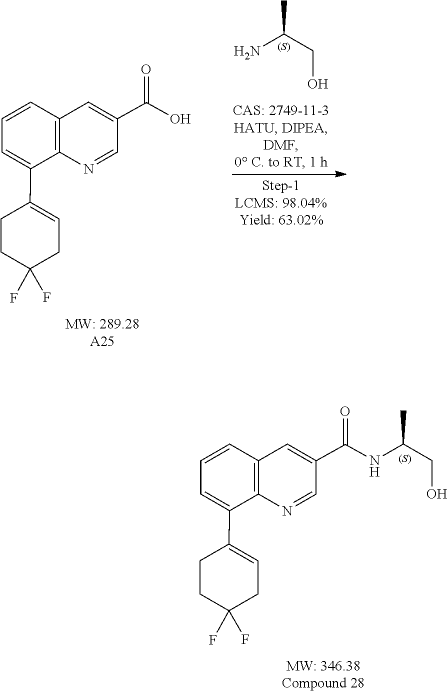

### Step-1:

A stirred solution of 8-(4,4-difluorocyclohex-1-en-1-yl)quinoline-3-carboxylic acid (A25, 0.1 g, 0.34 mmol, 1.0 eq) in DMF (1.0 mL) was prepared in 10 mL of glass vial at room temperature. To this reaction solution, DIPEA (0.18 mL, 1.03 mmol, 3.0 eq) and HATU (0.19 g, 0.51 mmol, 1.5 eq) were added at 0° C. under nitrogen atmosphere. After 30 min of stirring at 0° C., (S)-2-aminopropan-1-ol (CAS: 2749-11-3) (0.025 g, 0.34 mmol, 1.0 eq) was added at same temperature. Then the resulting reaction mixture was stirred from 0° C. to RT for 1 h. The reaction was monitored by TLC (using neat EtOAc as a mobile phase) which confirmed that the reaction got completed after 1 h of stirring from 0° C. to RT. The resulting reaction mixture was diluted with cold water (5 mL) and extracted with EtOAc (3×5 mL). The combined organic layers were dried over Na₂SO₄, filtered and concentrated under reduced pressure to afford 0.18 g crude. The obtained crude material was purified by flash column chromatography using silica (230-400 mesh) as a stationary phase (70% EtOAc in Hexane) yielding as a(S)-8-(4,4-difluorocyclohex-1-en-1-yl)-N-(1-hydroxypropan-2-yl)quinoline-3-carboxamide (Compound 28, 0.075 g, 0.21 mmol, Yield: 63.02%).

¹H NMR (DMSO-d₆, 400 MHz): δ ppm, 9.26 (d, J=2.4 Hz, 1H), 8.81 (d, J=2.0 Hz, 1H), 8.47 (d, J=7.6 Hz, 1H), 8.02 (dd, J=7.6, 2.4 Hz, 1H), 7.67-7.62 (m, 2H), 5.76 (br s, 1H), 4.79 (t, J=5.6 Hz, 1H), 4.10-4.06 (m, 1H), 3.53-3.48 (m, 1H), 3.42-3.37 (m, 1H), 2.89 (br s, 2H), 2.79 (t, J=14.8 Hz, 2H), 2.25-2.18 (m, 2H), 1.18 (d, J=6.4 Hz, 3H).

¹H NMR (MeOD, 400 MHz): δ ppm, 9.26 (d, J=2.4 Hz, 1H), 8.77 (d, J=2.0 Hz, 1H), 7.98 (dd, J=8.0, 1.6 Hz, 1H), 7.69-7.59 (m, 2H), 5.75 (br s, 1H), 4.32-4.24 (m, 1H), 3.68 (dd, J=17.6 Hz, 11.6 Hz, 2H), 3.67 (s, 1H), 3.01 (br s, 2H), 2.89-2.74 (m, 2H), 2.34-2.24 (m, 2H), 1.39-1.28 (m, 3H). (Note: —NH and —OH proton might exchange with deuterium from MeOD)

LCMS (Method A): 1.970 min, 98.04%, 254 nm, MS: ES+347.2 (M+1)

HPLC (Method B): 7.064 min, 97.63%, 254 nm

Chiral HPLC (Method C): 2.79 min, 99.18%, 248 nm

### Example 25—Synthesis of 8-(4,4-difluorocyclohex-1-en-1-yl)-N-(oxazol-2-ylmethyl)quinoline-3-carboxamide (Compound 29)

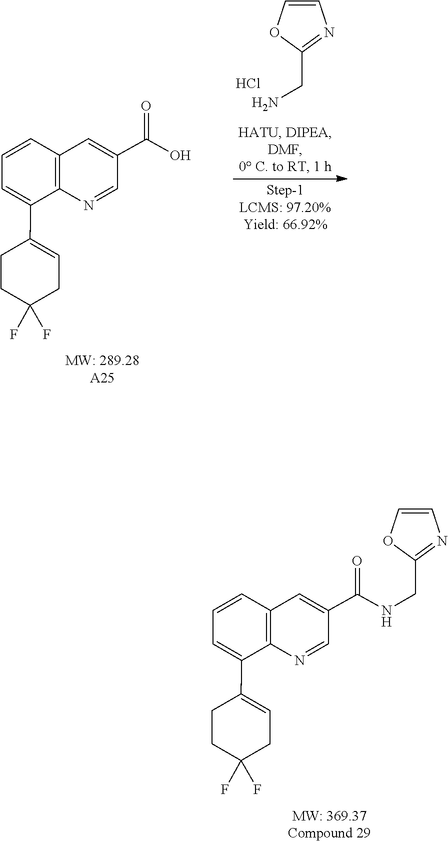 [SMILES: Cl ; NCc1ncco1 ; O=C(O)c1cnc2c(C3=CCC(F)(F)CC3)cccc2c1 ; O=C(NCc1ncco1)c1cnc2c(C3=CCC(F)(F)CC3)cccc2c1]

### Step-1:

A stirred solution of 8-(4,4-difluorocyclohex-1-en-1-yl)quinoline-3-carboxylic acid (A25, 0.1 g, 0.34 mmol, 1.0 eq) in DMF (1.0 mL) was prepared in 10 mL of glass vial at room temperature. To this reaction solution, DIPEA (0.30 mL, 1.72 mmol, 5.0 eq) and HATU (0.19 g, 0.51 mmol, 1.5 eq) were added at 0° C. under nitrogen atmosphere. After 30 min of stirring at 0° C., oxazol-2-ylmethanamine hydrochloride (0.046 g, 0.34 mmol, 1.0 eq) was added at same temperature. Then the resulting reaction mixture was stirred from 0° C. to RT for 1 h. The reaction was monitored by TLC (using EtOAc; 10.0 as mobile phase) which confirmed that the reaction got completed after 1 h of stirring from 0° C. to RT. The resulting reaction mixture was diluted with cold water (5 mL) and extracted with EtOAc (3×5 mL). The combined organic layers were dried over Na₂SO₄, filtered and concentrated under reduced pressure to afford 0.13 g crude. The obtained crude material was purified by flash column chromatography using silica (230-400 mesh) as a stationary phase (50% EtOAc in Hexane) yielding as an 8-(4,4-difluorocyclohex-1-en-1-yl)-N-(oxazol-2-ylmethyl)quinoline-3-carboxamide (Compound 29, 0.085 g, 0.23 mmol, Yield: 66.92%).

¹H NMR (DMSO-d₆, 400 MHz): δ ppm, 9.53 (t, J=5.2 Hz, 1H), 9.28 (d, J=2.0 Hz, 1H), 8.87 (d, J=2.0 Hz, 1H), 8.09 (s, 1H), 8.04 (dd, J=7.6 Hz, 1.6 Hz, 1H), 7.69-7.63 (m, 2H), 7.19 (s, 1H), 5.77 (br s, 1H), 4.67 (d, J=5.6 Hz, 2H), 2.90 (br s, 2H), 2.79 (t, J=14.4 Hz, 2H), 2.27-2.17 (m, 2H).

LCMS (Method A): 2.111 min, 97.20%, 254 nm, MS: ES+370.1 (M+1)

HPLC (Method B): 7.544 min, 98.69%, 254 nm

### Example 26—Synthesis of 8-(4,4-dimethylcyclohex-1-en-1-yl)-N-isopropylquinoline-3-carboxamide (Compound 30)

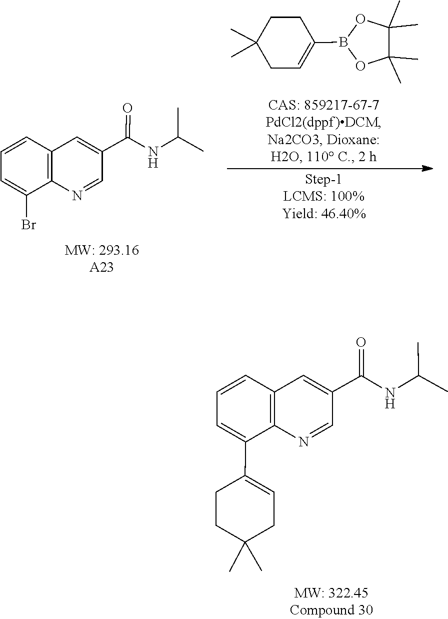

### Step-1:

A stirred solution of A23 (0.1 g, 0.34 mmol, 1 eq) in Dioxane:H₂O (3:1) were prepared in 10 mL glass vial at room temperature. To this reaction solution, CAS: 859217-67-7 (0.08 g, 0.33 mmol, 1 eq), Na₂CO₃ (0.10 g, 1.02 mmol, 3 eq) was added at same temperature. Purge by N₂. After that Pd(dppf)Cl₂ (0.02 g, 0.03 mmol, 0.1 eq) was added and stirred at 110° C. for 2 h. Then reaction was monitored by TLC (50% EtOAc:Hexane as mobile phase) which confirmed that the reaction got completed after 2 h of stirring at 110° C. The resulting reaction mixture was dilute with H₂O (20 mL) and extracted with EtOAc (20 mL). The organic layer was dried on Na₂SO₄, filtered and concentrate under high vacuum. The crude product was purification by combi-flash column chromatography using 230-400 mesh size silica. The product was eluted at 7% EtOAc:Hexane to yielding 8-(4,4-dimethylcyclohex-1-en-1-yl)-N-isopropylquinoline-3-carboxamide (Compound 30, 0.051 g, 0.158 mmol, Yield: 46.40%).

¹H NMR (DMSO-d₆, 400 MHz): δ ppm, 9.25 (d, J=2.0 Hz, 1H), 8.76 (d, J=2.0 Hz, 1H), 8.56 (d, J=7.6 Hz, 1H), 7.98-7.96 (m, 1H), 7.63-7.59 (m, 2H), 5.73 (br s, 1H), 4.20-4.11 (m, 1H), 2.61 (br s, 2H), 2.01 (br s, 2H), 1.52 (t, J=6.4 Hz, 2H), 1.21 (d, J=6.4 Hz, 6H), 1.05 (s, 6H).

¹H NMR (MeOD, 400 MHz): δ ppm, 9.22 (d, J=2 Hz, 1H), 8.71 (d, J=2.0 Hz, 1H), 7.93-7.90 (m, 1H), 7.62-7.59 (m, 2H), 5.78 (br s, 1H), 4.32-4.26 (m, 1H), 2.61 (s, 2H), 2.07-2.03 (m, 2H), 1.64 (t, J=6.4 Hz, 2H), 1.48 (d, J=6.8 Hz, 6H), 0.94 (s, 6H). (Note: —NH proton might exchange with deuterium from MeOD)

LCMS (Method A): 2.797 min, 100%, 254 nm, MS: ES+323.2 (M+1).

HPLC (Method B): 10.612 min, 100% at 254 nm.

### Example 27—Synthesis of(S)-8-(4,4-dimethylcyclohex-1-en-1-yl)-N-(1-(pyridin-2-yl)ethyl)quinoline-3-carboxamide (Compound 31)

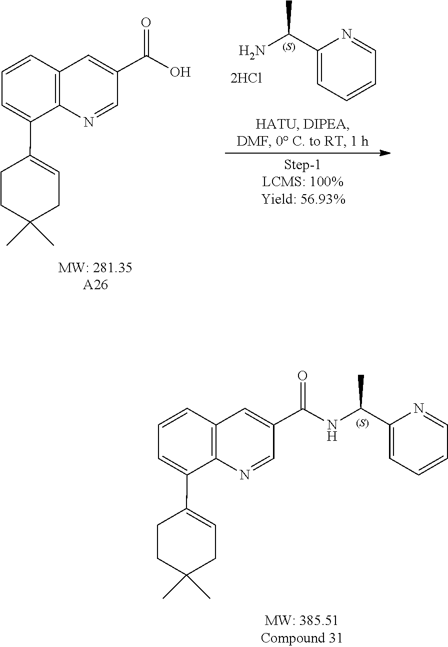

### Step-1:

A stirred solution of commercially available 8-(4,4-dimethylcyclohex-1-en-1-yl)quinoline-3-carboxylic acid (A26, 0.1 g, 0.35 mmol, 1.0 eq) in DMF (1.0 mL) was prepared in 10 mL of glass vial at room temperature. To this reaction solution, DIPEA (0.30 mL, 1.03 mmol, 5.0 eq) and HATU (0.20 g, 0.53 mmol, 1.5 eq) were added at 0° C. under nitrogen atmosphere. After 30 min of stirring at 0° C., (S)-1-(Pyridin-2-yl) Ethan amine hydrochloride (CAS: 40154-78-7) (0.069 g, 0.35 mmol, 1.0 eq) was added at same temperature. Then the resulting reaction mixture was stirred from 0° C. to RT for 1 h. The reaction was monitored by TLC (using neat EtOAc as a mobile phase) which confirmed that the reaction got completed after 1 h of stirring from 0° C. to RT. The resulting reaction mixture was diluted with cold water (5 mL) and extracted with EtOAc (3×15 mL). The combined organic layers were dried over Na₂SO₄, filtered and concentrated under reduced pressure to afford 0.14 g crude. The obtained crude material was purified by flash column chromatography using silica (230-400 mesh) as a stationary phase (40% EtOAc in Hexane) yielding as a(S)-8-(4,4-dimethylcyclohex-1-en-1-yl)-N-(1-(pyridin-2-yl)ethyl)quinoline-3-carboxamide (Compound 31, 0.078 g, 0.20 mmol, Yield: 56.93%)

¹H NMR (DMSO-d₆, 400 MHz): δ ppm, 9.31 (d, J=2.0 Hz, 1H), 9.18 (d, J=7.6 Hz, 1H), 8.86 (d, J=2.0 Hz, 1H), 8.54 (d, J=4.0 Hz, 1H), 8.00-7.98 (m, 1H), 7.77 (dt, J=7.6 Hz, 1.6 Hz, 1H), 7.64-7.61 (m, 2H), 7.47 (d, J=8.0 Hz, 1H), 7.29-7.26 (m, 1H), 5.74 (br s, 1H), 5.27-5.24 (m, 1H), 2.62 (br s, 2H), 2.01 (br s, 2H), 1.56-1.51 (m, 5H), 1.05 (s, 6H).

LCMS (Method A): 2.560 min, 100%, 254 nm, MS: ES+386.2 (M+1)

HPLC (Method B): 10.376 min, 100%, 254 nm

Chiral HPLC (Method C): 9.687 min, 100%, 240 nm

### Example 28—Synthesis of(S)-8-(4,4-dimethylcyclohex-1-en-1-yl)-N-(1-hydroxypropan-2-yl)quinoline-3-carboxamide (Compound 32)

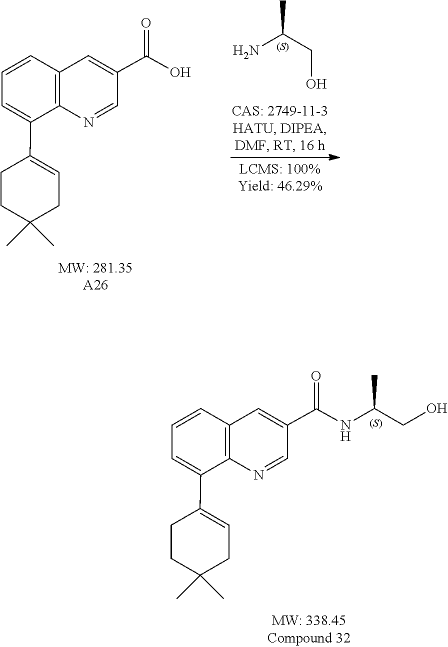

### Step-1:

A stirred solution of A26 (0.090 g, 0.310 mmol, 1.0 eq) in DMF (1.0 mL) was prepared in 10 mL of glass vial at room temperature. To this reaction solution, DIPEA (0.124 g, 0.950 mmol, 3.0 eq) and HATU (0.18 g, 0.470 mmol, 1.5 eq) were added at same temperature under nitrogen atmosphere. After 10 min of stirring, (S)-2-aminopropan-1-ol (CAS: 2749-11-3) (0.024 g, 0.310 mmol, 1.0 eq) was added at same temperature. Then the resulting reaction mixture was stirred at RT for 16 h. The reaction was monitored by TLC (using Neat ethyl acetate as mobile phase) which confirmed that the reaction got completed after 16 h of stirring at RT. The resulting reaction mixture was poured in cold water (40 mL) and extracted with ethyl acetate (50×2) organic dried over Na₂SO₄ and concentrated under reduce pressure to obtain crude product which was purified by prep TLC to yielding as a(S)-8-(4,4-dimethylcyclohex-1-en-1-yl)-N-(1-hydroxypropan-2-yl)quinoline-3-carboxamide (Compound 32, 0.050 g, 0.147 mmol, Yield: 46.29%).

¹H NMR (DMSO-d₆, 400 MHz): δ ppm, 9.26 (s, 1H), 8.78 (s, 1H), 8.45 (d, J=8.0 Hz, 1H), 7.97 (t, J=4.8 Hz, 1H), 7.63-7.60 (m, 2H), 5.74 (br s, 1H), 4.79 (t, J=5.6 Hz, 1H), 4.11-4.04 (m, 1H), 3.53-3.47 (m, 1H), 3.41-3.35 (m, 1H), 2.61 (br s, 2H), 2.01 (br s, 2H), 1.52 (t, J=6.4 Hz, 2H), 1.17 (d, J=6.8 Hz, 3H), 1.05 (s, 6H).

LCMS (Method A): 2.411 min, 100%, 254.0 nm, MS: ES+339.11 (M+1)

HPLC (Method B): 8.906 min, 100%, 254.0 nm

Chiral HPLC (Method B): 4.01 min, 100%, 260.0 nm

### Example 29—Synthesis of 8-(4,4-dimethylcyclohex-1-en-1-yl)-N-(oxazol-2-ylmethyl)quinoline-3-carboxamide (Compound 33)

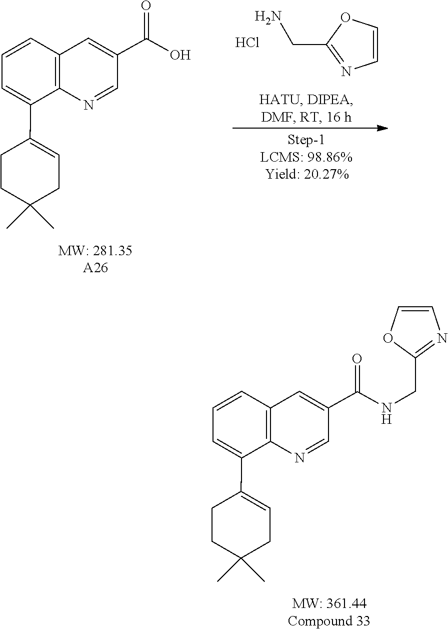 [SMILES: CC1(C)CC=C(c2cccc3cc(C(=O)O)cnc23)CC1 ; Cl ; NCc1ncco1 ; CC1(C)CC=C(c2cccc3cc(C(=O)NCc4ncco4)cnc23)CC1]

### Step-1:

A stirred solution of A26 (0.1 g, 0.35 mmol, 1 eq) in DMF (1 mL) were prepared in 10 mL glass vial at room temperature. To this reaction solution was added DIPEA (0.19 g, 1.053 mmol, 3 eq) and HATU (0.20 g, 0.53 mmol, 1.5 eq) at 0° C. and stirred for 10 min. Then oxazol-2-ylmethanamine (0.03 g, 0.30 mmol, 1 eq) was added and resulting reaction mixture was stirred at RT for 16 h. The reaction was monitored by TLC (EtOAc:Hexane 7.0:3.0 as mobile phase) which confirmed that the reaction got completed after 16 h of stirring at RT. The resulting reaction mixture was extracted by EtOAc (20 mL) and H₂O (20 mL). The organic layer was dried on Na₂SO₄, filtered and concentrate under high vacuum to obtained crude product. The crude product was purification by combi-flash column chromatography using 230-400 mesh size silica and product was eluted at 50% EtOAc:Hexane to yielding 8-(4-4-dimethylcyclohex-1-en-1-yl)-N-(oxazol-2-ylmethyl)quinoline-3-carboxamide (Compound 33, 0.026 g, 0.074 mmol, Yield: 20.27%).

¹H NMR (DMSO-d₆, 400 MHz): δ ppm, 9.51 (br s, 1H), 9.27 (s, 1H), 8.84 (s, 1H), 8.08 (s, 1H), 7.98 (t, J=4.4 Hz 1H), 7.62 (d, J=4.4 Hz, 2H), 7.19 (s, 1H), 5.74 (br s, 1H), 4.66 (d, J=5.2 Hz, 2H), 2.67-2.33 (m, 2H), 2.01 (br s, 2H), 1.52 (t, J=5.6 Hz, 2H), 1.04 (s, 6H).

¹H NMR (MeOD, 400 MHz): δ ppm 9.28 (s, 1H), 8.79 (d, J=2.0 Hz, 1H), 7.94 (t, J=4.4 Hz, 2H), 7.66-7.61 (m, 2H), 7.18 (s, 1H), 5.78 (br s, 1H), 4.66 (s, 2H), 2.62 (s, 2H), 2.04 (t, J=10.0 Hz, 2H), 1.64 (t, J=6.4 Hz, 2H), 1.10 (s, 6H). (Note: —NH proton might exchange with deuterium from MeOD)

LCMS (Method A): 2.569 min, 98.86% at 254 nm, MS: ES+362 (M+1)

HPLC (Method B): 9.413 min, 100% at 254 nm.

### Example 30—Synthesis of 8-(cyclohex-1-en-1-yl)-N-isopropylquinoline-3-carboxamide (Compound 34)

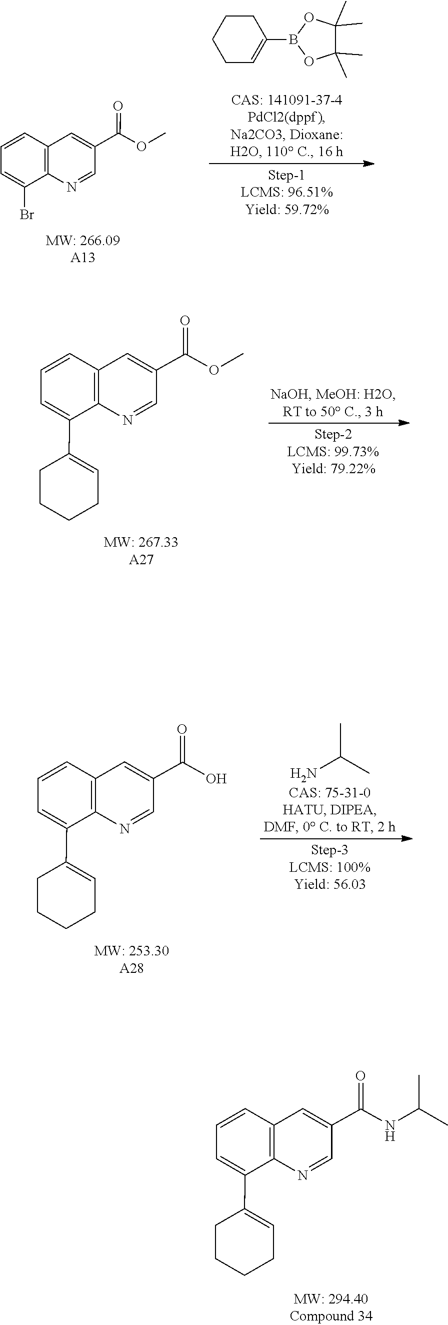

### Step-1:

A stirred solution of A13 (1.0 g, 3.750 mmol, 1.0 eq), and CAS: 141091-37-4 (0.78 g, 3.750 mmol, 1.0 eq) in Dioxane:water (4:1) were prepared in 35 mL glass vial at room temperature. To this reaction solution, Na₂CO₃ (1.19 g, 11.20 mmol, 3.0 eq) was added at same temperature. Then the resulting reaction mixture was stirred at RT and nitrogen purging for 15 min. After purging nitrogen gas added PdCl₂ (dppf) at RT then the reaction was stirred at 110° C. for 16 h. The reaction was monitored by TLC (20% ethyl acetate in hexane as mobile phase) which confirmed that the reaction got completed after 16 h of stirring at 110° C. Then resulting reaction mixture was cooled at RT and poured in cold water (200 mL). Then again extracted with ethyl acetate (2×200 mL) organic dried over Na₂SO₄, filtered and concentrated under reduce pressure to obtain crude material which was purified by column chromatography using hexane and ethyl acetate. Product was eluted at 7% ethyl acetate in hexane to afford methyl 8-(cyclohex-1-en-1-yl)quinoline-3-carboxylate (A27, 0.6 g, 2.244 mmol, Yield: 59.72%).

¹H NMR (DMSO-d₆, 400 MHz): δ ppm, 9.30 (d, J=2.0 Hz, 1H), 8.99 (d, J=2.0 Hz, 1H), 8.10 (dd, J=8.0 Hz, J=2.0 Hz, 1H), 7.69-7.63 (m, 2H), 5.83 (br s, 1H), 3.95 (s, 3H), 2.57 (br s, 2H), 2.22 (br s, 2H), 1.78-1.70 (m, 4H).

LCMS (Method A): 2.821 min, 96.51%, 254.0 nm, MS: ES+268.10 (M+1)

### Step-2:

A stirred solution of A27 (0.6 g, 2.240 mmol, 1.0 eq) in MeOH:H₂O (4.2:1.8 mL) was prepared in 35 mL glass vial at room temperature. To this reaction solution, NaOH (0.179 g, 4.480 mmol, 2.0 eq), were added at same temperature. Then, the resulting reaction mixture was stirred at 50° C. for 3 h. The reaction was monitored by TLC (using neat ethyl acetate as mobile phase) which confirmed that the reaction got completed after 3 h of stirring at RT. The resulting reaction mixture was directly concentrated under reduce pressure then added water (15 mL) after acidify with citric acid to obtain white solid. The precipitate was filtered through buckler funnel and washed with water (20 mL) and dried over high vacuum to yielding 8-(cyclohex-1-en-1-yl)quinoline-3-carboxylic acid (A28, 0.45 g, 1.776 mmol, Yield: 79.22%).

¹H NMR (DMSO-d₆, 400 MHz): δ ppm, 13.54 (br s, 1H), 9.29 (d, J=2.0 Hz, 1H), 8.93 (d, J=2.0 Hz, 1H), 8.07 (dd, J=7.6 Hz, J=2.0 Hz, 1H), 7.66-7.13 (m, 2H), 5.83 (br s, 1H), 2.58 (br s, 2H), 2.22 (br s, 2H), 1.77-1.70 (m, 4H).

LCMS (Method A): 2.220 min, 99.73%, 254.0 nm, MS: ES+254.10 (M+1)

### Step-3:

A stirred solution of A28 (0.1 g, 0.390 mmol, 1.0 eq) in DMF (1.0 mL) was prepared in 10 mL of glass vial at room temperature. To this reaction solution, DIPEA (0.153 g, 1.180 mmol, 3.0 eq) and HATU (0.225 g, 0.590 mmol, 1.5 eq) were added at same temperature under nitrogen atmosphere. After 10 min of stirring, propan-2-amine (CAS: 75-31-0) (0.023 g, 0.390 mmol, 1.0 eq) was added at 0° C. Then the resulting reaction mixture was stirred at RT for 16 h. The reaction was monitored by TLC (using 50% ethyl acetate in hexane as mobile phase) which confirmed that the reaction got completed after 16 h of stirring at RT. The resulting reaction mixture was poured in cold water (40 mL) and extracted with ethyl acetate (2×40 mL) organic dried over Na₂SO₄ and concentrated under reduce pressure to obtain crude product which was purified by normal column chromatography using hexane and ethyl acetate product was eluted at 30% ethyl acetate in hexane to yielding as a 8-(cyclohex-1-en-1-yl)-N-isopropylquinoline-3-carboxamide (Compound 34, 0.065 g, 0.220 mmol, Yield: 56.03%).

¹H NMR (DMSO-d₆, 400 MHz): δ ppm, 9.23 (d, J=2.0 Hz, 1H), 8.76 (d, J=2.0 Hz, 1H), 8.56 (d, J=7.6 Hz, 1H), 7.96 (t, J=4.8 Hz, 1H), 7.63-7.60 (m, 2H), 5.83 (br s, 1H), 4.18-4.11 (m, 1H), 2.67 (br s, 2H), 2.22 (br s, 2H), 1.77-1.70 (m, 4H), 1.21 (d, J=6.8 Hz, 6H).

LCMS (Method A): 2.397 min, 100%, 254.0 nm, MS: ES+295.16 (M+1)

HPLC (Method B): 9.316 min, 99.74%, 254.0 nm

### Example 31—Synthesis of(S)-8-(cyclohex-1-en-1-yl)-N-(1-(pyridin-2-yl)ethyl)quinoline-3-carboxamide (Compound 35)

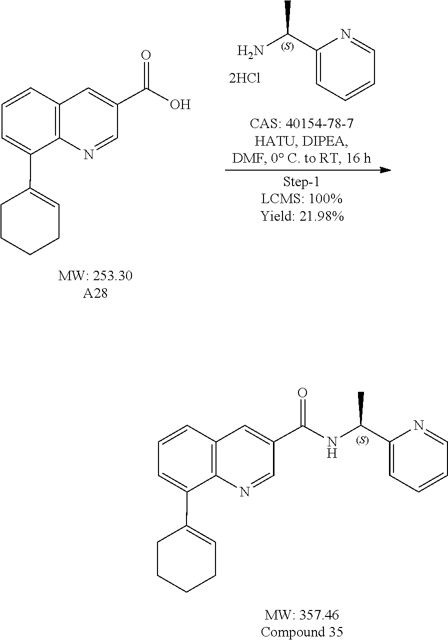

### Step-1:

A stirred solution of A28 (0.1 g, 0.390 mmol, 1.0 eq) in DMF (1.0 mL) was prepared in 10 mL of glass vial at room temperature. To this reaction solution, DIPEA (0.255 g, 1.900 mmol, 5.0 eq) and HATU (0.225 g, 0.590 mmol, 1.5 eq) were added at same temperature under nitrogen atmosphere. After 10 min of stirring, CAS: 40154-78-7 (0.077 g, 0.390 mmol, 1.0 eq) was added at 0° C. Then the resulting reaction mixture was stirred at RT for 16 h. The reaction was monitored by TLC (using neat ethyl acetate as mobile phase) which confirmed that the reaction got completed after 16 h of stirring at RT. The resulting reaction mixture was poured in cold water (40 mL) and extracted with ethyl acetate (2×40 mL) organic dried over Na₂SO₄ and concentrated under reduce pressure to obtain crude product which was purified by normal column chromatography using hexane and ethyl acetate product was eluted at 35% ethyl acetate in hexane further purified by prep TLC to yielding as a(S)-8-(cyclohex-1-en-1-yl)-N-(1-(pyridin-2-yl)ethyl)quinoline-3-carboxamide (Compound 35, 0.031 g, 0.086 mmol, Yield: 21.98%).

¹H NMR (DMSO-d₆, 400 MHz): δ ppm, 9.28 (d, J=2.0 Hz, 1H), 9.19 (d, J=7.6 Hz, 1H), 8.87 (d, J=2.0 Hz, 1H), 8.54 (d, J=4.4 Hz, 1H) 7.98 (t, J=4.8 Hz, 1H), 7.78 (dt, J=7.6 Hz, 1.6 Hz, 1H), 7.62 (d, J=4.8 Hz, 2H), 7.47 (d, J=8.0 Hz, 1H), 7.29-7.26 (m, 1H), 5.84 (br s, 1H), 5.29-5.22 (m, 1H), 2.59 (br s, 2H), 2.33 (br s, 2H), 1.77-1.70 (m, 4H), 1.55 (d, J=7.2 Hz, 3H).

LCMS (Method A): 2.131 min, 100%, 210 nm, MS: ES+358.12 (M+1)

HPLC (Method B): 9.162 min, 99.49%, 254.0 nm

Chiral HPLC (Method C): 2.88 min, 98.97%, 241.0 nm

### Example 32—Synthesis of(S)-8-(cyclohex-1-en-1-yl)-N-(1-hydroxypropan-2-yl)quinoline-3-carboxamide (Compound 36)

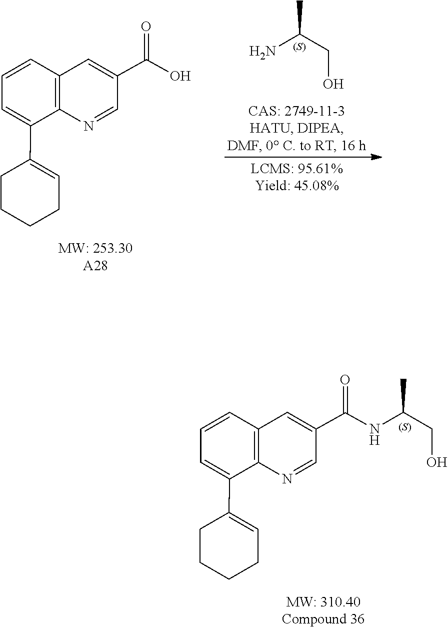

### Step-1:

### Procedure:

A stirred solution of A28 (0.1 g, 0.390 mmol, 1.0 eq) in DMF (1.0 mL) was prepared in 10 mL of glass vial at room temperature. To this reaction solution, DIPEA (0.153 g, 1.110 mmol, 3.0 eq) and HATU (0.225 g, 0.590 mmol, 1.5 eq) were added at same temperature under nitrogen atmosphere. After 10 min of stirring, (S)-2-aminopropan-1-ol (CAS: 2749-11-3) (0.030 g, 0.390 mmol, 1.0 eq) was added at 0° C. Then the resulting reaction mixture was stirred at RT for 16 h. The reaction was monitored by TLC (using neat ethyl acetate as mobile phase) which confirmed that the reaction got completed after 16 h of stirring at RT. The resulting reaction mixture was poured in cold water (35 mL) and extracted with ethyl acetate (2×50 mL) organic dried over Na₂SO₄ and concentrated under reduce pressure to obtain crude product which was purified by normal column chromatography using hexane and ethyl acetate product was eluted at 50% ethyl acetate in hexane to yielding as a(S)-8-(cyclohex-1-en-1-yl)-N-(1-hydroxypropan-2-yl)quinoline-3-carboxamide (Compound 36, 0.055 g, 0.177 mmol, Yield: 45.08%).

¹H NMR (DMSO-d₆, 400 MHz): δ ppm, 9.24 (d, J=2.0 Hz, 1H), 8.78 (d, J=2.0 Hz, 1H), 8.46 (d, J=8.0 Hz, 1H), 7.96 (t, J=4.8 Hz, 1H), 7.63-7.60 (m, 2H), 5.83 (br s, 1H), 4.79 (t, J=6.0 Hz, 1H), 4.11-4.04 (m, 1H), 3.53-3.48 (m, 1H), 3.41-3.37 (m, 1H), 2.59 (br s, 2H), 2.22 (br s, 2H), 1.77-1.70 (m, 4H), 1.17 (d, J=6.4 Hz, 3H).

LCMS (Method A): 2.006 min, 95.61%, 254 nm, MS: ES+311.11 (M+1)

HPLC (Method B): 7.558 min, 97.76%, 254.0 nm

Chiral HPLC (Method C): 2.85 min, 100%, 241.0 nm

### Example 33—Synthesis of 8-(cyclohex-1-en-1-yl)-N-(oxazol-2-ylmethyl)quinoline-3-carboxamide (Compound 37)

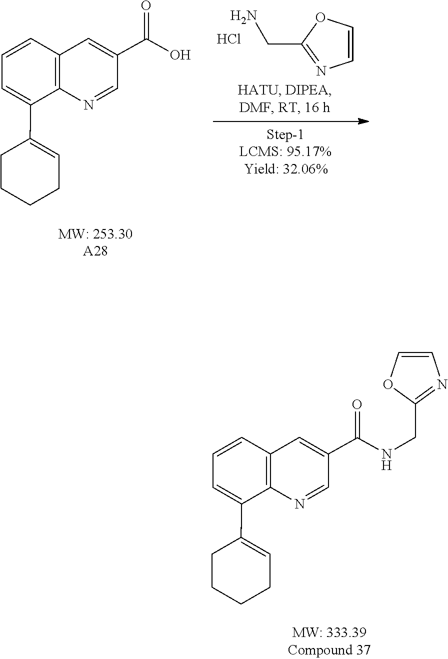 [SMILES: O=C(O)c1cnc2c(C3=CCCCC3)cccc2c1 ; Cl ; NCc1ncco1 ; O=C(NCc1ncco1)c1cnc2c(C3=CCCCC3)cccc2c1]

### Step-1:

A stirred solution of A28 (0.1 g, 0.39 mmol, 1 eq) in DMF (1 mL) were prepared in 10 mL glass vial at room temperature. To this reaction solution was added DIPEA (0.15 g, 1.8 mmol, 3 eq), HATU (0.22 g, 0.58 mmol, 1.5 eq) at 0° C. and stirred for 10 min. Then oxazol-2-ylmethanamine (0.03 g, 0.39 mmol, 1 eq) was added and resulting reaction mixture was stirred at RT for 16 h. The reaction was monitored by TLC (MeOH:DCM, 1.0:9.0 as mobile phase) which confirmed that the reaction got completed after 16 h of stirring at RT. The resulting reaction mixture was extracted by EtOAc (20 mL) and H₂O (20 mL). The organic layer was dried on Na₂SO₄, filtered and concentrated under high vacuum to obtained crude product. The crude product was purification by combi-flash column chromatography using 230-400 mesh size silica and product was eluted at 12% EtOAc:Hexane yielding as an 8-(cyclohex-1-en-1-yl)-N-(oxazol-2-ylmethyl)quinoline-3-carboxamide. (Compound 37, 0.042 g, 0.125 mmol, Yield: 32.06%).

¹H NMR (DMSO-d₆, 400 MHz): δ ppm, 9.52 (t, J=5.2 Hz, 1H), 9.27 (d, J=2.0 Hz, 1H), 8.84 (d, J=2.0 Hz, 1H), 8.09 (s, 1H), 7.99-7.97 (m, 1H), 7.62 (d, J=4.0 Hz, 2H), 7.19 (s, 1H), 5.84 (br s, 1H), 4.67 (d, J=5.2 Hz, 2H), 2.58 (br s, 2H), 2.22 (br s, 2H), 1.77-1.70 (m, 4H).

¹H NMR (MeOD, 400 MHz): δ ppm, 9.28 (d, J=2.4 Hz, 1H), 8.81 (d, J=2.4 Hz, 1H), 7.95 (t, J=6.0 Hz, 2H), 7.67-7.62 (m, 2H), 7.18 (s, 1H), 5.86 (br s, 1H), 4.79 (s, 2H), 2.56 (d, J=1.6 Hz, 2H), 2.31-2.29 (m, 2H), 1.89-1.86 (m, 2H), 1.83-1.80 (m, 2H). (Note: —NH proton might exchange with deuterium from MeOD)

LCMS (Method A): 2.209 min, 95.17%, 254 nm, MS: ES+334.1 (M+1)

HPLC (Method B): 8.202 min, 96.41%, 254 nm

### Example 34—Synthesis of N-methyl-8-(4-(trifluoromethyl)cyclohex-1-en-1-yl)quinoline-3-carboxamide (Compound 38)

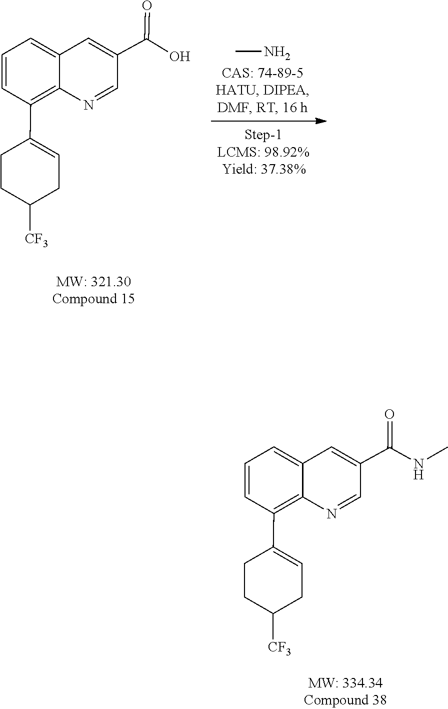

### Step-1:

To a solution 8-(4-(trifluoromethyl)cyclohex-1-en-1-yl)quinoline-3-carboxylic acid (Compound 15, 0.09 g, 0.280 mmol, 1.0 eq), HATU (0.159 g, 0.420 mmol, 1.5 eq) and DIPEA (0.14 ml, 0.840 mmol, 3.0 eq) in DMF (1 mL) at 0° C. under nitrogen atmosphere stirred for 10 min, was added CAS: 74-89-5 (0.021 g, 0.420 mmol, 1.5 eq) under Nitrogen. The resulting mixture was stirred for 16 h at room temperature. The reaction was monitored on TLC (using EtOAc:Hex; 8:2 as mobile phase) which confirmed that the reaction got completed after 16 h. The resulting reaction mixture was quenched with ice-cold water (10 mL) and extracted with EtOAc (3×20 mL). The combined organic layers were dried over Na₂SO₄ and concentrated under reduced pressure to afford 0.15 g crude. The obtained crude material was purified by flash column chromatography using silica (230-400 mesh) as stationary phase (50% Ethyl acetate in hexane) yielding N-methyl-8-(4-(trifluoromethyl)cyclohex-1-en-1-yl)quinoline-3-carboxamide (Compound 38, 0.035 g, 0.104 mmol, Yield: 37.38%).

¹H NMR (DMSO-d₆, 400 MHz): δ ppm, 9.24 (d, J=2.4 Hz, 1H), 8.80-8.74 (m, 2H), 8.01-7.99 (dd, J=6.0 Hz, =4.0 Hz, 1H), 7.64-7.61 (m, 2H), 5.86 (br s, 1H), 2.87 (d, J=4.8 Hz, 3H), 2.87-2.59 (m, 1H), 2.80-2.50 (m, 2H), 2.33-2.24 (m, 2H), 2.33-2.07 (m, 1H), 1.74-1.64 (m, 1H) (Note: -one CF₃—CH proton merge with DMSO solvent peak and clearly visible in MeOD NMR)

¹H NMR (MeOD, 400 MHz): δ ppm, 9.24 (d, J=2.0 Hz, 1H), 8.74 (t, J=2.4 Hz, 1H), 7.96 (dd, J=7.6, 2.0 Hz, 1H), 7.69-7.63 (m, 2H), 5.86 (br s, 1H), 3.02 (s, 3H), 2.76-2.64 (m, 1H), 2.55-2.33 (m, 2H), 2.51 (s, 1H), 2.18-2.03 (m, 1H), 1.92-1.82 (br s, 1H), 1.92-1.82 (m, 1H) (Note: —NH proton might exchange with deuterium from MeOD)

LCMS (Method A): 2.327 min, 98.92%, 254.0 nm, MS: ES+429.3 (M+1)

HPLC (Method A): 8.561 min, 97.93%, 210.0 nm

CHIRAL HPLC (Method A): Peak-1 3.73 min, 49.03%, 240.0 nm; Peak-2 4.02 min, 50.02%, 240.0 nm

### Example 35—Synthesis of N-(2-hydroxyethyl)-8-(4-(trifluoromethyl)cyclohex-1-en-1-yl)quinoline-3-carboxamide (Compound 39)

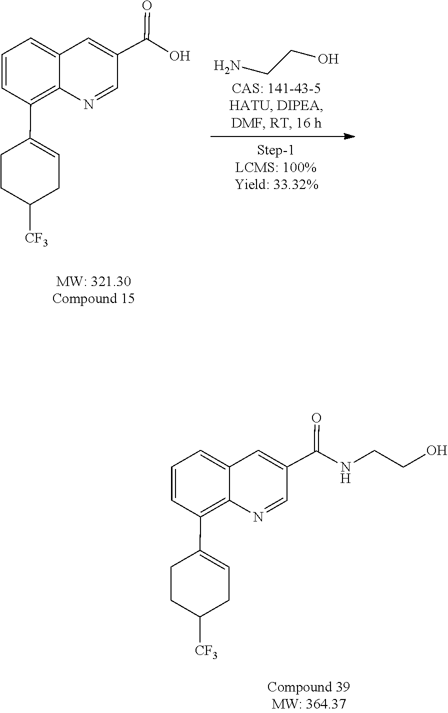

### Step-1:

To a solution 8-(4-(trifluoromethyl)cyclohex-1-en-1-yl)quinoline-3-carboxylic acid (Compound 15, 0.09 g, 0.280 mmol, 1.0 eq), HATU (0.159 g, 0.420 mmol, 1.5 eq) and DIPEA (0.14 ml, 0.840 mmol, 3.0 eq) in DMF (1 mL) at 0° C. under nitrogen atmosphere stirred for 10 min, was added CAS: 141-43-5 (0.025 g, 0.420 mmol, 1.5 eq) under Nitrogen. The resulting mixture was stirred for 16 h at room temperature. The reaction was monitored on TLC (using EtOAc:Hex; 8:2 as mobile phase) which confirmed that the reaction got completed after 16 h. The resulting reaction mixture was quenched with ice-cold water (10 mL) and extracted with EtOAc (3×20 mL). The combined organic layers were dried over Na₂SO₄ and concentrated under reduced pressure to afford 0.15 g crude. The obtained crude material was purified by flash column chromatography using silica (230-400 mesh) as stationary phase (80% Ethyl acetate in hexane as elution gradient) yielding N-(2-hydroxyethyl)-8-(4-(trifluoromethyl)cyclohex-1-en-1-yl)quinoline-3-carboxamide (Compound 39, 0.034 g, 0.093 mmol, Yield: 33.32%).

¹H NMR (DMSO-d₆, 400 MHz): δ ppm, 9.25 (d, J=2.0 Hz, 1H), 8.81-8.80 (m, 2H), 8.00 (J=5.6, 4.7 Hz, 1H), 7.64-7.61 (m, 2H), 5.87 (br s, 1H), 4.80 (t, J=5.6 Hz, 1H), 3.59-3.54 (q, J=6.0 Hz, 2H), 3.42-3.34 (q, J=6.0 Hz, 2H), 2.80-2.77 (m, 1H), 2.72-2.67 (m, 2H), 2.32-2.24 (m, 1H), 2.10-2.07 (m, 1H), 1.74-1.64 (m, 1H) (Note: -one CF₃—CH proton merge with DMSO solvent peak and clearly visible in MeOD NMR)

¹H NMR (MeOD, 400 MHz): δ ppm, 9.26 (d, J=2.0 Hz, 1H), 8.78 (d, J=2.4 Hz, 1H), 7.97 (dd, J=7.6, 2 Hz, 1H), 7.69-7.63 (m, 2H), 5.86 (s, 1H), 3.78 (t, J=5.6 Hz, 2H), 3.59 (t, J=5.6 Hz, 2H), 2.78-2.65 (m, 3H), 2.55-2.51 (m, 1H), 2.40-2.37 (m, 1H), 2.18-2.15 (m, 1H), 1.91-1.85 (m, 1H). (Note: —NH and —OH proton might exchange with deuterium from MeOD)

LCMS (Method A): 2.154 min, 100%, 254.0 nm, MS: ES+365 (M+1)

HPLC (Method A): 7.651 min, 99.49%, 210.0 nm

CHIRAL HPLC (Method A): Peak-1 4.25 min, 49.65%, 240.0 nm; 4.69 min, 49.98%, 240.0 nm

### Example 36—Synthesis of N-(2-methoxyethyl)-8-(4-(trifluoromethyl)cyclohex-1-en-1-yl)quinoline-3-carboxamide (Compound 40)

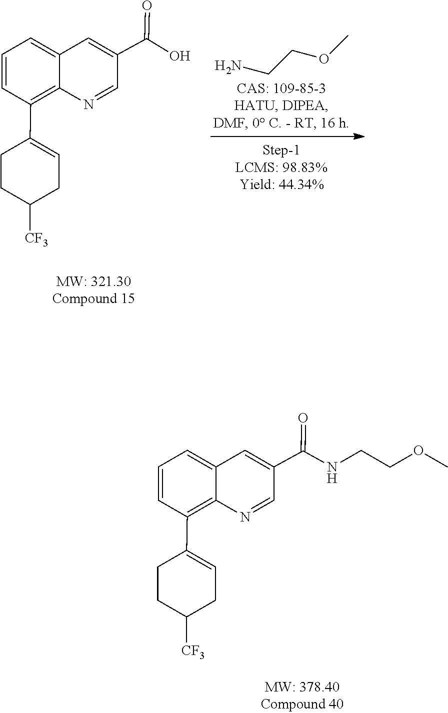

### Step-1

To a solution of 8-(4-(trifluoromethyl)cyclohex-1-en-1-yl)quinoline-3-carboxylic acid (Compound 15, 0.09 g, 0.280 mmol, 1.0 eq), HATU (0.160 g, 0.420 mmol, 1.5 eq) and DIPEA (0.15 mL, 0.840 mmol, 3.0 eq) in DMF (2 mL) at 0° C. under nitrogen atmosphere stirred for 20 min, was added CAS: 109-85-3 (0.0315 g, 0.420 mmol, 1.5 eq) under Nitrogen. The resulting mixture was stirred for 16 h at room temperature. The reaction was monitored on TLC (using EtOAc:Hexane; 7.0:3.0 as mobile phase) which confirmed that the reaction got completed after 16 h. The resulting reaction mixture was quenched with ice-cold water (10 mL) and extracted with EtOAc (3×15 mL). The combined organic layers were dried over Na₂SO₄ and concentrated under reduced pressure to afford 0.08 g crude. The obtained crude material was purified by flash column chromatography using silica (230-400 mesh size) as stationary phase (elution gradient 50% Ethyl acetate in hexane) yielding N-(2-methoxyethyl)-8-(4-(trifluoromethyl)cyclohex-1-en-1-yl)quinoline-3-carboxamide (Compound 40, 0.047 g, 0.124 mmol, Yield: 44.34%)

¹H NMR (DMSO-d₆, 400 MHz): δ ppm, 9.25 (d, J=2.0 Hz, 1H), 8.90 (br s, 1H), 8.81 (d, J=2.0 Hz, 1H), 8.02-7.99 (m, 1H), 7.65-7.62 (m, 2H), 5.87 (br s, 1H), 3.51 (s, 4H), 3.30 (s, 3H), 2.81-2.69 (m, 3H), 2.29-2.25 (m, 1H), 2.09-2.07 (m, 1H), 1.74-1.64 (m, 1H) (Note: -one CF₃—CH proton merge with DMSO solvent peak and clearly visible in MeOD NMR)

¹H NMR (MeOD, 400 MHz): δ ppm, 9.25 (d, J=2.0 Hz, 1H), 8.77 (d, J=2.4 Hz, 1H), 7.97 (dd, J=1.6, 7.6 Hz, 1H), 7.69-7.63 (m, 2H), 5.87 (br s, 1H), 3.67 (s, 4H), 3.42 (s, 3H), 2.83-2.65 (m, 3H), 2.55-2.51 (m, 1H), 2.41-2.34 (m, 1H), 2.18-2.15 (m, 1H), 1.93-1.82 (m, 1H) (Note: —NH proton might exchange with deuterium from MeOD)

LCMS (Method A): 2.393 min, 98.83%, 254.0 nm, MS: ES+379.1 (M+1)

HPLC (Method A): 8.672 min, 97.52%, 254.0 nm

Chiral HPLC: Peak-1: 5.14 min, 49.67%, 240 nm; Peak-2: 5.57 min, 50.32%, 240 nm

### Example 37—Synthesis of N-isopropyl-N-methyl-8-(4-(trifluoromethyl)cyclohex-1-en-1-yl)quinoline-3-carboxamide (Compound 41)

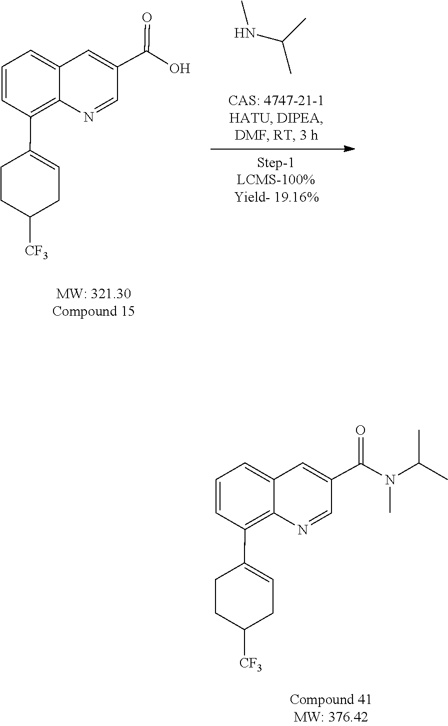

### Step-1:

To a stirred solution of 8-(4-(trifluoromethyl)cyclohex-1-en-1-yl)quinoline-3-carboxylic acid (Compound 15, 0.09 g, 1.2 mmol, 1.0 eq), CAS: 4747-21-1 (0.024 g, 0.336 mmol, 1.2 eq) and DIPEA (0.10 g, 0.84 mmol, 3.0 eq) in DMF (2 mL) under inert condition N₂ (g) was added HATU (0.159 g, 0.42 mmol, 1.5 eq). The reaction was monitored by TLC (using ethyl acetate in hexane (3:7) as mobile phase) which confirmed that the reaction got completed after 3 h of stirring at room temperature. The resulting reaction mixture was diluted with water (10 mL) and extracted with EtOAc (3×20 mL). The combined organic layers extracted with EtOAc (2×50 mL), dried over Na₂SO₄ and concentrated under reduced pressure to afford 0.07 g crude. The crude was purified by reverse phase HPLC yielding N-Isopropyl-N-methyl-8-(4-(trifluoromethyl)cyclohex-1-en-1-yl)quinoline-3-carboxamide (Compound 41, 0.023 g, 0.061 mmol, Yield 19.16%)

¹H VT-NMR (DMSO-d₆, 400 MHz): δ ppm, 8.86 (br s, 1H), 8.36 (br s, 1H), 7.95 (br s, 1H), 7.61 (br s, 1H), 5.89 (br s, 2H), 4.28 (br s, 1H), 2.89-2.70 (m, 6H), 2.34-2.31 (m, 1H), 2.12-2.10 (m, 1H), 1.75-1.73 (m, 1H), 1.26-1.19 (br s, 6H). (Note: -one CF₃—CH proton merge with DMSO solvent peak and clearly visible in MeOD NMR)

¹H-NMR (MeOD, 400 MHz): δ ppm, 8.87 (d, J=12.4 Hz, 1H), 8.40 (d, J=17.2 Hz, 1H), 7.94 (dd, J=6.8, 7.2 Hz, 1H), 7.67-7.62 (m, 2H), 5.86 (br s, 1H), 4.04-3.95 (m, 1H), 3.36-2.95 (s, 3H), 2.79-2.62 (m, 3H) 2.55-2.50 (m, 1H), 2.40-2.36 (m, 1H), 2.18-2.15 (m, 1H), 1.92-1.86 (m, 1H), 1.32 (d, J=6.8 Hz, 3H) 1.27 (d, J=6.8 Hz, 3H)

¹H NMR (DMSO-d₆, 400 MHz): δ ppm, 8.87 (d, J=12.4 Hz, 1H), 8.46 (br s, 1H), 8.40 (br s, 1H), 7.98-7.96 (m, 1H), 7.64-7.61 (m, 3H), 5.85 (s, 2H), 4.78 (br s, 1H), 3.86 (br s, 1H), 2.90-2.60 (m, 9H), 2.33-2.23 (m, 1H), 2.08-2.05 (m, 1H), 1.74-1.63 (m, 1H), 1.23-1.14 (m, 6H) (Note: Due to rotamers peaks observed broad)

LCMS (Method-A): 2.512 min, (100%), 254 nm, m/z=377.17 (M+H)⁺

HPLC (Method-A): 9.81 min, (100%), 210 nm

### Example 38—Synthesis of N-(pyrimidin-2-ylmethyl)-8-(4-(trifluoromethyl)cyclohex-1-en-1-yl)quinoline-3-carboxamide (Compound 42)

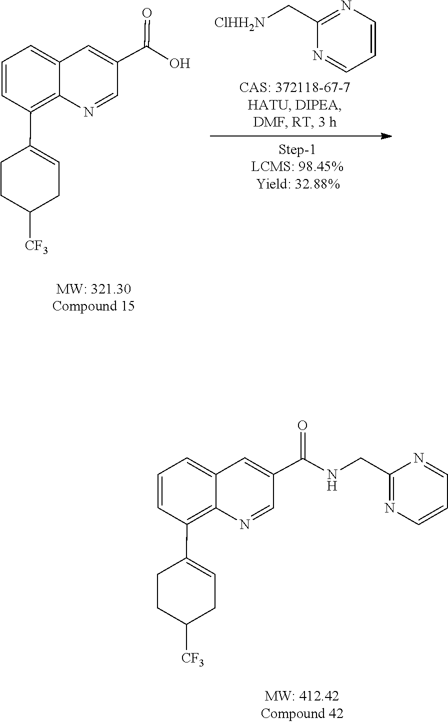

### Step-1:

To a stirred solution of 8-(4-(trifluoromethyl)cyclohex-1-en-1-yl)quinoline-3-carboxylic acid (Compound 15, 0.01 g, 0.31 mmol, 1.0 eq), CAS: 372118-67-7 (0.045 g, 0.31 mmol, 1.0 eq) and DIPEA (0.12 g, 0.93 mmol, 3.0 eq) in DMF (2 mL) under inert condition N₂(g) was added HATU (0.178 g, 0.46 mmol, 1.5 eq). The reaction was monitored by TLC (using MeOH in DCM (0.5:9.5) as mobile phase) which confirmed that the reaction got completed after 3 h of stirring at room temperature. The resulting reaction mixture was diluted with water (10 mL) and extracted with EtOAc (3×20 mL). The combined organic layers extracted with EtOAc (2×50 mL). dried over Na₂SO₄ and concentrated under reduced pressure to afford 0.06 g crude. The crude was purified by manual column chromatography using silica (230-400 mesh) as stationary phase (gradient elution 50% methanol in DCM) yielding Compound 42 (0.039 g, 0.094 mmol, Yield 32.88%)

¹H-NMR (DMSO-d₆, 400 MHz): δ ppm 9.45 (t, J=6.0 Hz, 1H), 9.31 (d, J=2.4 Hz, 1H), 8.88 (d, J=2.4 Hz, 1H), 8.79 (d, J=4.8 Hz, 2H), 8.04-8.01 (m, 1H), 7.66-7.63 (m, 2H), 7.43 (t, J=4.8 Hz, 1H), 5.88 (br s, 1H), 4.74 (d, J=6.0 Hz, 2H), 2.82-2.80 (m, 1H), 2.79-2.67 (m, 2H), 2.40-2.25 (m, 2H), 2.10-2.07 (m, 1H), 1.73-1.66 (m, 1H) (Note: -one CF₃—CH proton merge with DMSO solvent peak and clearly visible in MeOD NMR)

¹H-NMR (MeOD, 400 MHz): δ ppm, 9.32 (d, J=2.0 Hz, 1H), 8.87 (d, J=2.0 Hz, 1H), 8.80 (d, J=5.2 Hz, 2H), 8.00 (dd, J=8.0, 7.6 Hz, 1H), 7.71-7.64 (m, 2H), 7.43 (t, J=4.8 Hz, 1H), 5.88 (br s, 1H), 2.79-2.76 (m, 1H), 2.69-2.66 (m, 2H), 2.56-2.51 (m, 1H), 2.39-2.33 (m, 1H), 2.18-2.16 (m, 1H) 1.93-1.83 (m, 1H), (Note: —NH proton might exchange with deuterium from MeOD; 2H merged with MeOD moisture peak)

LCMS (Method-A): 2.28 min, (98.45%), 254 nm; MS: ES+413 (M+1)

HPLC (Method-A): 8.20 min, (97.7%), 210 nm

### Example 39—Synthesis of N-(pyridazin-3-ylmethyl)-8-(4-(trifluoromethyl)cyclohex-1-en-1-yl)quinoline-3-carboxamide (Compound 43)

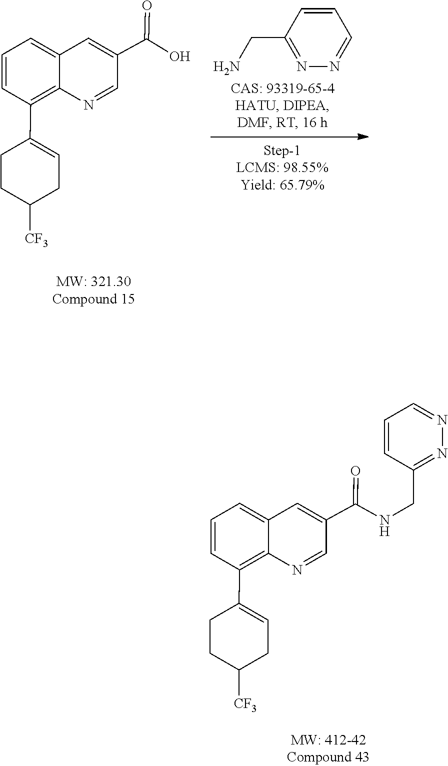 [SMILES: O=C(O)c1cnc2c(C3=CCC(C(F)(F)F)CC3)cccc2c1 ; NCc1cccnn1 ; O=C(NCc1cccnn1)c1cnc2c(C3=CCC(C(F)(F)F)CC3)cccc2c1]

### Step-1:

To a solution 8-(4-(trifluoromethyl)cyclohex-1-en-1-yl)quinoline-3-carboxylic acid (Compound 15, 0.09 g, 0.280 mmol, 1.0 eq), HATU (0.159 g, 0.420 mmol, 1.5 eq) and DIPEA (0.14 ml, 0.840 mmol, 3.0 eq) in DMF (1 mL) at 0° C. under nitrogen atmosphere stirred for 10 min, was added CAS: 93319-65-4 (0.036 g, 0.336 mmol, 1.2 eq) under Nitrogen. The resulting mixture was stirred for 16 h at room temperature. The reaction was monitored on TLC (using MDC:MeOH; 9:1 as mobile phase) which confirmed that the reaction got completed after 16 h. The resulting reaction mixture was quenched with ice-cold water (10 mL) and extracted with EtOAc (3×20 mL). The combined organic layers were dried over Na₂SO₄ and concentrated under reduced pressure to afford 0.15 g crude. The obtained crude material was purified by flash column chromatography using silica (230-400 mesh size) as stationary phase (5% MeOH in DCM) yielding N-(2-hydroxyethyl)-8-(4-(trifluoromethyl)cyclohex-1-en-1-yl)quinoline-3-carboxamide (Compound 43, 0.076 g, 0.184 mmol, Yield: 65.79%).

¹H NMR (DMSO-d₆, 400 MHz): δ ppm, 9.60 (t, J=5.2 Hz, 1H), 9.31 (d, J=2.0 Hz, 1H), 9.16 (d, J=2.4 Hz, 1H), 8.88 (d, J=2.0 Hz, 1H), 8.03-8.01 (m, 1H), 7.73-7.63 (m, 4H), 5.87 (br s, 1H), 4.85 (d, J=5.6 Hz, 2H), 2.85-2.71 (m, 2H), 2.28-2.25 (m, 1H), 2.09-2.07 (m, 1H), 1.74-1.67 (m, 1H). (Note: -one CF₃—CH proton merge with DMSO solvent peak and clearly visible in MeOD NMR)

¹H NMR (MeOD, 400 MHz): δ ppm, 9.31 (d, J=2.4 Hz, 1H), 9.14 (d, J=4.0 Hz, 1H), 8.85 (d, J=2.4 Hz, 1H), 7.98 (dd, J=1.2 Hz, 1H), 7.85 (d, J=7.6 Hz, 1H), 7.77-7.74 (m, 1H), 7.69-7.64 (m, 2H), 5.87 (br s, 1H), 4.95 (s, 2H), 2.79-2.76 (m, 1H), 2.69-2.65 (m, 2H), 2.55-2.51 (m, 1H), 2.41-2.33 (m, 1H), 2.18-2.15 (m, 1H), 1.88-1.82 (m, 1H). (Note: —NH proton might exchange with deuterium from MeOD)

LCMS (Method A): 2.243 min, 98.55%, 254.0 nm, MS: ES+413 (M+1)

HPLC (Method A): 7.95 min, 98.09%, 254.0 nm

CHIRAL HPLC (Method A): Peak-1 4.25 min, 49.65%, 240.0 nm;

Peak-2 4.69 min, 49.98%, 240.0 nm

### Example 40—Synthesis of N-(pyrazin-2-ylmethyl)-8-(4-(trifluoromethyl)cyclohex-1-en-1-yl)quinoline-3-carboxamide (Compound 44)

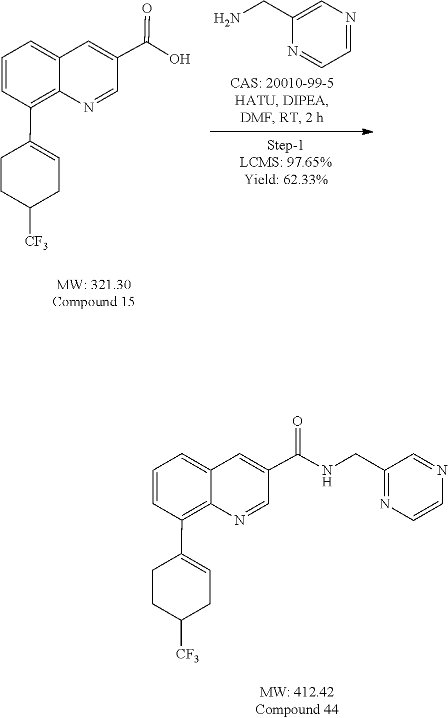 [SMILES: O=C(O)c1cnc2c(C3=CCC(C(F)(F)F)CC3)cccc2c1 ; NCc1cnccn1 ; O=C(NCc1cnccn1)c1cnc2c(C3=CCC(C(F)(F)F)CC3)cccc2c1]

### Step-1

To a solution of 8-(4-(trifluoromethyl)cyclohex-1-en-1-yl)quinoline-3-carboxylic acid (Compound 15, 0.09 g, 0.280 mmol, 1.0 eq), HATU (0.160 g, 0.420 mmol, 1.5 eq) and DIPEA (0.15 mL, 0.840 mmol, 3.0 eq) in DMF (2 mL) at 0° C. under nitrogen atmosphere stirred for 20 min, was added CAS: 20010-99-5 (0.046 g, 0.420 mmol, 1.5 eq) under Nitrogen. The resulting mixture was stirred for 2 h at room temperature. The reaction was monitored on TLC (using 100% EtOAc as mobile phase) which confirmed that the reaction got completed after 2 h. The resulting reaction mixture was quenched with ice-cold water (10 mL) and extracted with EtOAc (3×15 mL). The combined organic layers were dried over Na₂SO₄ and concentrated under reduced pressure to afford 0.072 g crude. The obtained crude material was purified by flash column chromatography using silica (230-400 mesh size) as stationary phase (eluent gradient 95% ethyl acetate in hexane) yielding N-(pyrazin-2-ylmethyl)-8-(4-(trifluoromethyl)cyclohex-1-en-1-yl)quinoline-3-carboxamide (Compound 44, 0.072 g, 0.174 mmol, Yield: 62.33%).

¹H NMR (DMSO-d₆, 400 MHz): δ ppm, 9.54 (t, J=5.6 Hz, 1H), 9.30 (d, J=2.0 Hz, 1H), 8.88 (d, J=1.6 Hz, 1H), 8.73 (s, 1H), 8.62 (s, 1H), 8.57 (d, J=2.0 Hz, 1H), 8.03-8.01 (m, 1H), 7.66-7.63 (m, 2H), 5.87 (br s, 1H), 4.71 (d, J=5.6 Hz, 2H), 2.81-2.68 (m, 3H), 2.32-2.25 (m, 1H), 2.10-2.07 (m, 1H), 1.75-1.67 (m, 1H). (Note: -one CF₃—CH proton merge with DMSO solvent peak and clearly visible in MeOD NMR)

¹H NMR (MeOD, 400 MHz): δ ppm, 9.30 (d, J=2.0 Hz, 1H), 8.83 (d, J=2.0 Hz, 1H), 8.75 (s, 1H), 8.62 (s, 1H), 8.55 (d, J=2.0 Hz, 1H), 7.98 (dd, J=1.6, 8.0 Hz, 1H), 7.70-7.64 (m, 2H), 5.87 (br s, 1H), 4.87 (s, 2H), 2.79-2.79-2.77 (m, 1H), 2.69-2.66 (m, 2H), 2.56-2.51 (m, 1H), 2.41-2.34 (m, 1H), 2.18-2.15 (m, 1H), 1.93-1.85 (m, 1H). (Note: —NH proton might exchange with deuterium from MeOD)

LCMS (Method-A): 2.305 min, 98.22%, 254.0 nm, MS: ES+413.2 (M+1)

HPLC (Method-A): 8.360 min, 97.65%, 254.0 nm

### Example 41—Synthesis of N-((5-methyloxazol-2-yl)methyl)-8-(4-(trifluoromethyl)cyclohex-1-en-1-yl)quinoline-3-carboxamide (Compound 45)

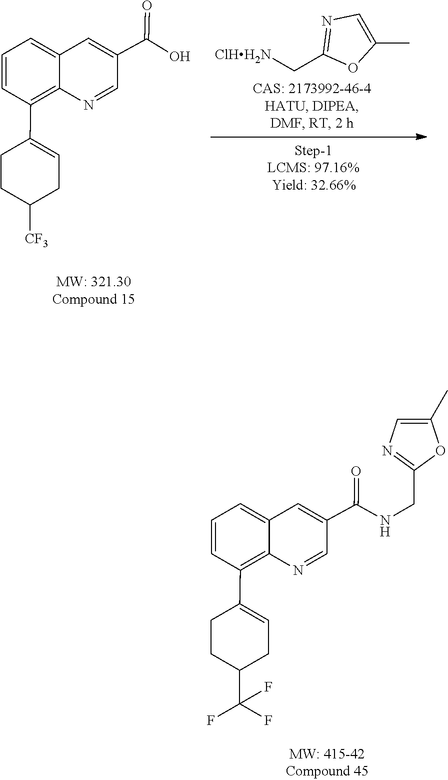

### Step-1

To a solution of 8-(4-(trifluoromethyl)cyclohex-1-en-1-yl)quinoline-3-carboxylic acid (Compound 15, 0.09 g, 0.280 mmol, 1.0 eq), HATU (0.160 g, 0.420 mmol, 1.5 eq) and DIPEA (0.15 mL, 0.840 mmol, 3.0 eq) in DMF (2 mL) at 0° C. under nitrogen atmosphere stirred for 20 min, was added CAS: 2173992-46-4 (0.0457 g, 0.420 mmol, 1.5 eq) under Nitrogen. The resulting mixture was stirred for 2 h at room temperature. The reaction was monitored on TLC (using 5% MeOH:DCM as mobile phase) which confirmed that the reaction got completed after 2 h. The resulting reaction mixture was quenched with ice-cold water (10 mL) and extracted with EtOAc (3×15 mL). The combined organic layers were dried over Na₂SO₄ and concentrated under reduced pressure to afford 0.08 g crude. The obtained crude material was purified by flash column chromatography using silica (230-400 mesh) as stationary phase (5% MeOH in DCM) yielding N-((5-methyloxazol-2-yl)methyl)-8-(4-(trifluoromethyl)cyclohex-1-en-1-yl)quinoline-3-carboxamide (Compound 45, 0.038 g, 0.0914 mmol, Yield: 32.66%)

¹H NMR (DMSO-d₆, 400 MHz): δ ppm, 9.49 (br s, 1H), 9.27 (s, 1H), 8.86 (s, 1H), 8.02 (d, J=3.6 Hz, 1H), 7.66 (s, 2H), 6.79 (s, 1H), 5.87 (br s, 1H), 4.60 (d, J=4.8 Hz, 2H), 2.81-2.68 (m, 3H), 2.32-2.28 (m, 4H), 2.10-2.07 (m, 1H), 1.71-1.67 (m, 1H) (Note: CF₃—CH proton merge with DMSO solvent peak which is clearly observed in MeOD NMR)

¹H NMR (MeOD, 400 MHz): δ ppm, 9.29 (s, 1H), 8.83 (s, 1H), 7.98 (d, J=7.2 Hz, 1H), 7.70-7.64 (m, 2H), 6.78 (s, 1H), 5.87 (br s, 1H), 4.73 (s, 2H), 2.79-2.66 (m, 3H), 2.56-2.51 (m, 1H), 2.41-2.34 (m, 4H), 2.18-2.15 (m, 1H), 1.91-1.85 (m, 1H). (Note: —NH proton might exchange with deuterium from MeOD)

LCMS (Method-A): 2.433 min, 97.16%, 254.0 nm, MS: ES+416.1 (M+1)

HPLC (Method-A): 8.81 min, 97.37%, 254.0 nm

### Example 42—Synthesis of N-(2-hydroxy-1-(pyridin-2-yl)ethyl)-8-(4-(trifluoromethyl)cyclohex-1-en-1-yl)quinoline-3-carboxamide (Compound 46)

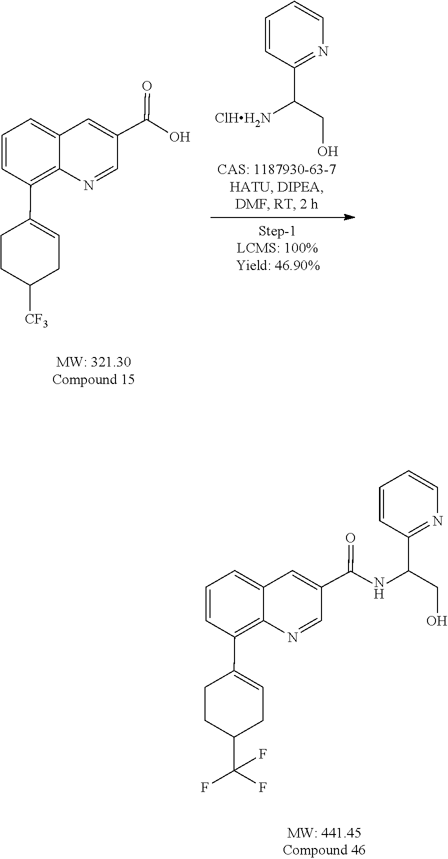

### Step-1

To a solution of 8-(4-(trifluoromethyl)cyclohex-1-en-1-yl)quinoline-3-carboxylic acid (Compound 15, 0.09 g, 0.280 mmol, 1.0 eq), HATU (0.160 g, 0.420 mmol, 1.5 eq) and DIPEA (0.15 mL, 0.840 mmol, 3.0 eq) in DMF (2 mL) at 0° C. under nitrogen atmosphere stirred for 20 min, was added CAS: 1187930-63-7 (0.065 g, 0.308 mmol, 1.1 eq) under Nitrogen. The resulting mixture was stirred for 2 h at room temperature. The reaction was monitored on TLC (using MeOH:DCM; 0.5:9.5 as mobile phase) which confirmed that the reaction got completed after 2 h. The resulting reaction mixture was quenched with ice-cold water (10 mL) and extracted with EtOAc (3×15 mL). The combined organic layers were dried over Na₂SO₄ and concentrated under reduced pressure to afford 0.08 g crude. The obtained crude material was purified by flash column chromatography using silica (230-400 mesh size) as stationary phase (elution gradient 95% Ethyl acetate in hexane) yielding N-(2-hydroxy-1-(pyridin-2-yl)ethyl)-8-(4-(trifluoromethyl)cyclohex-1-en-1-yl)quinoline-3-carboxamide (Compound 46, 0.058 g, 0.131 mmol, Yield: 46.90%)

¹H NMR (DMSO-d₆, 400 MHz): δ ppm, 9.30 (s, 1H), 9.09 (d, J=7.6 Hz, 1H), 8.92 (s, 1H), 8.55 (d, J=4.0 Hz, 1H), 8.03 (t, J=4.8 Hz, 1H), 7.78 (t, J=6.8 Hz, 1H), 7.66 (d, J=4.4 Hz, 2H), 7.49 (d, J=8.0 Hz, 1H), 7.29 (t, J=5.6 Hz, 1H), 5.88 (br s, 1H), 3.90-3.82 (m, 2H), 2.81-2.68 (m, 4H), 2.33-2.26 (m, 1H), 2.10-2.08 (m, 1H), 1.72-1.68 (m, 1H). (Note: CF₃—CH proton merge with DMSO solvent peak which is clearly observed in MeOD NMR)

¹H NMR (MeOD, 400 MHz): δ ppm, 9.30 (d, J=2.0 Hz, 1H), 8.89 (d, J=2.4 Hz, 1H), 8.59 (d, J=4.4 Hz, 1H), 8.00 (dd, J=2.0, 8.0 Hz, 1H), 7.86 (dt, J=1.6, 8.0 Hz, 1H), 7.70-7.65 (m, 2H), 7.57 (d, J=8.0 Hz, 1H), 7.38-7.35 (m, 1H), 5.88 (br s, 1H), 5.39 (t, J=6.4 Hz, 1H), 4.09-3.99 (m, 2H), 2.83-2.66 (m, 3H), 2.56-2.52 (m, 1H), 2.41-2.34 (m, 1H), 2.19-2.16 (m, 1H), 1.93-1.86 (m, 1H). (Note: —NH and —OH proton might exchange with deuterium from MeOD)

LCMS (Method-A): 2.171 min, 100%, 210.0 nm, MS: ES+442.2 (M+1)

HPLC (Method-A): 8.19 min, 99.16%, 254.0 nm

### Example 43—Synthesis of N-((1H-pyrazol-5-yl)methyl)-8-(4-(trifluoromethyl)cyclohex-1-en-1-yl)quinoline-3-carboxamide (Compound 47)

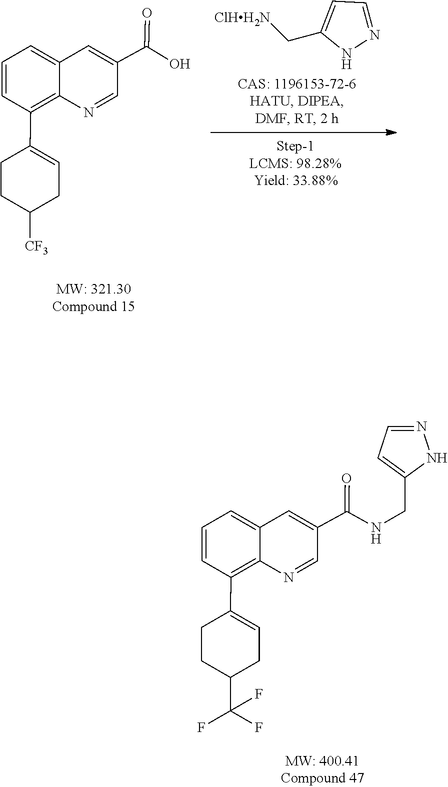

### Step-1

To a solution of 8-(4-(trifluoromethyl)cyclohex-1-en-1-yl)quinoline-3-carboxylic acid (Compound 15, 0.09 g, 0.280 mmol, 1.0 eq), HATU (0.160 g, 0.420 mmol, 1.5 eq) and DIPEA (0.15 mL, 0.840 mmol, 3.0 eq) in DMF (2 mL) at 0° C. under nitrogen atmosphere stirred for 20 min, was added CAS: 1196153-72-6 (0.041 g, 0.308 mmol, 1.1 eq) under Nitrogen. The resulting mixture was stirred for 2 h at room temperature. The reaction was monitored on TLC (using MeOH:DCM; 0.5:9.5 as mobile phase) which confirmed that the reaction got completed after 2 h. The resulting reaction mixture was quenched with ice-cold water (10 mL) and extracted with EtOAc (3×15 mL). The combined organic layers were dried over Na₂SO₄ and concentrated under reduced pressure to afford 0.08 g crude. The obtained crude material was purified by flash column chromatography using silica (230-400 mesh size) as stationary phase (elution gradient 5% MeOH in DCM) yielding N-((1H-pyrazol-5-yl)methyl)-8-(4-(trifluoromethyl)cyclohex-1-en-1-yl)quinoline-3-carboxamide (Compound 47, 0.038 g, 0.0949 mmol, Yield: 33.88%)

¹H NMR (DMSO-d₆, 400 MHz) VT NMR: δ ppm, 12.47 (s, 1H), 9.29 (s, 1H), 8.99 (s, 1H), 8.81 (s, 1H), 7.97 (d, J=6.8 Hz, 1H), 7.64-7.61 (m, 3H), 6.24 (s, 1H), 5.89 (br s, 1H), 4.58 (d, J=4.8 Hz, 2H), 2.82-2.70 (m, 3H), 2.35-2.28 (m, 1H), 2.13-2.10 (m, 1H), 1.80-1.71 (m, 1H) (Note: -one CF₃—CH proton merge with DMSO solvent peak and clearly visible in MeOD NMR)

¹H NMR (MeOD, 400 MHz): δ ppm, 9.28 (d, J=2.4 Hz, 1H), 8.80 (d, J=2.0 Hz, 1H), 7.97 (dd, J=1.6, 8.0 Hz, 1H), 7.69-7.63 (m, 3H), 6.38 (s, 1H), 5.87 (br s, 1H), 4.71 (s, 2H), 2.78-2.65 (m, 3H), 2.56-2.51 (m, 1H), 2.41-2.33 (m, 1H), 2.18-2.15 (m, 1H), 1.93-1.82 (m, 1H). (Note: —NH proton might exchange with deuterium from MeOD)

LCMS (Method-A): 2.257 min, 98.28%, 254.0 nm, MS: ES+401.2 (M+1)

HPLC (Method-A): 8.02 min, 95.17%, 254.0 nm

### Example 44—Synthesis of N-((1H-1,2,4-triazol-3-yl)methyl)-8-(4-(trifluoromethyl)cyclohex-1-en-1-yl)quinoline-3-carboxamide (Compound 48)

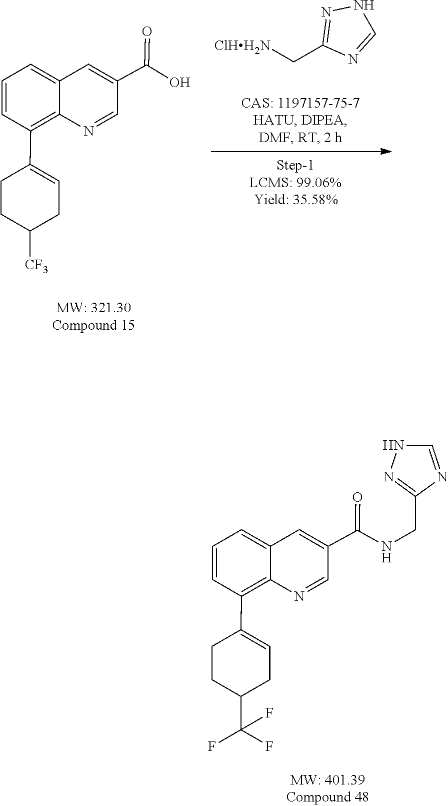

### Step-1

To a solution of 8-(4-(trifluoromethyl)cyclohex-1-en-1-yl)quinoline-3-carboxylic acid (Compound 15, 0.09 g, 0.280 mmol, 1.0 eq), HATU (0.160 g, 0.420 mmol, 1.5 eq) and DIPEA (0.15 mL, 0.840 mmol, 3.0 eq) in DMF (2 mL) at 0° C. under nitrogen atmosphere stirred for 20 min, was added CAS: 1197157-75-7 (0.041 g, 0.308 mmol, 1.1 eq) under Nitrogen. The resulting mixture was stirred for 2 h at room temperature. The reaction was monitored on TLC (using MeOH:DCM; 0.5:9.5 as mobile phase) which confirmed that the reaction got completed after 2 h. The resulting reaction mixture was quenched with ice-cold water (10 mL) and extracted with EtOAc (3×15 mL). The combined organic layers were dried over Na₂SO₄ and concentrated under reduced pressure to afford 0.07 g crude. The obtained crude material was purified by flash column chromatography using silica (230-400 mesh size) as stationary phase (elution gradient 5% MeOH in DCM) yielding N-((1H-1,2,4-triazol-3-yl)methyl)-8-(4-(trifluoromethyl)cyclohex-1-en-1-yl)quinoline-3-carboxamide (Compound 48, 0.040 g, 0.0996 mmol, Yield: 35.58%)

¹H NMR (DMSO-d₆, 400 MHz) VT NMR: δ ppm, 13.70 (s, 1H), 9.29 (d, J=1.6 Hz, 1H), 9.13 (bs, 1H), 8.83 (d, J=1.6 Hz, 1H), 8.44 (br s, 1H), 7.98 (d, J=6.4 Hz, 1H), 7.67-7.61 (m, 2H), 5.91 (br s, 1H), 4.66 (s, 2H), 3.54-3.46 (m, 1H), 2.83-2.68 (m, 4H), 2.35-2.28 (m, 1H), 2.13-2.11 (m, 1H), 1.80-1.71 (m, 1H) (Note: -one CF₃—CH proton merge with DMSO solvent peak and clearly visible in MeOD NMR)

¹H NMR (MeOD, 400 MHz): δ ppm, 9.30 (s, 1H), 8.84 (s, 1H), 8.48 (br s, 1H), 7.98 (d, J=7.6 Hz, 1H), 7.70-7.63 (m, 2H), 5.87 (br s, 1H), 4.81 (s, 2H), 2.79-2.65 (m, 3H), 2.56-2.51 (m, 1H), 2.41-2.33 (m, 1H), 2.18-2.16 (m, 1H), 1.93-1.83 (m, 1H). (Note: —NH proton might exchange with deuterium from MeOD)

LCMS (Method-A): 2.069 min, 99.06%, 254.0 nm, MS: ES+402.2 (M+1)

HPLC (Method-A): 6.22 min, 98.60%, 254.0 nm

### Example 45—Synthesis of N-(cyanomethyl)-8-(4-(trifluoromethyl)cyclohex-1-en-1-yl)quinoline-3-carboxamide (Compound 49)

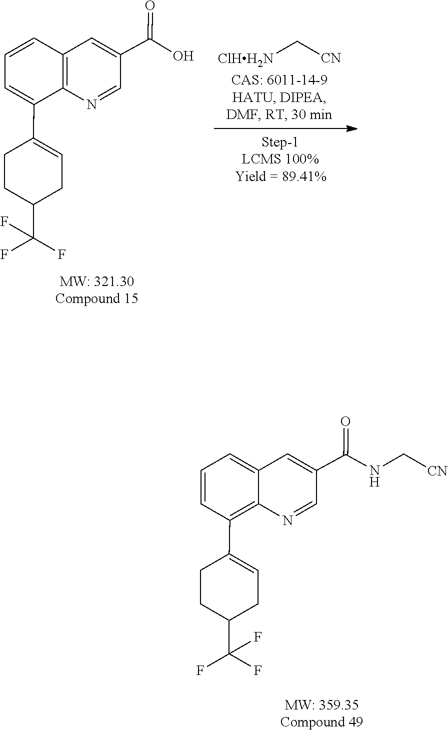

### Step-1:

To a solution of 8-(4-(triflouromethyl)cyclohex-1-en-1-yl)quinoline-3-carboxylic acid (Compound 15, 0.1 g, 0.3112 mmol, 1.0 eq), HATU (0.177 g, 0.466 mmol, 1.5 eq) and DIPEA (0.161 g, 1.244 mmol, 4.0 eq) in DMF (1 mL) at 0° C. under nitrogen atmosphere stirred for 20 min, was added CAS: 6011-14-9 (0.03168 g, 0.342 mmol, 1.1 eq) under Nitrogen. The resulting mixture was stirred for 30 min at room temperature. The reaction was monitored on TLC (using EtOAc:Hexane; 1:1 as mobile phase) which confirmed that the reaction got completed after 30 min. The resulting reaction mixture was quenched with ice-cold water (50 mL) and extracted with EtOAc (3×15 mL). The combined organic layers were dried over Na₂SO₄ and concentrated under reduced pressure to afford 0.15 g crude. The obtained crude material was purified by flash column chromatography using silica (230-400 mesh size) as stationary phase (30% Ethyl acetate in hexane) yielding N-(Cyanomethyl)-8-(4-(triflouromethyl)cyclohex-1-en-1-yl)quinoline-3-carboxamide (Compound 49, 0.1 g, 0.278 mmol, Yield: 89.41%)

¹H NMR (DMSO d⁶, 400 MHz): δ ppm, 9.58 (t, J=5.2 Hz, 1H), 9.25 (d, J=2.0 Hz, 1H), 8.87 (d, J=2.0 Hz, 1H), 8.04 (dd, J=7.2 Hz, 2.8 Hz, 7.2 Hz, 1H), 7.69-7.64 (m, 2H), 5.88 (s, 1H), 4.43 (d, J=5.6 Hz, 2H), 2.81-2.567 (m, 3H), 2.33-2.25 (m, 1H), 2.09 (d, J=11.6, 1H), 1.75-1.64 (m, 1H) (Note: -one CF₃—CH proton merge with DMSO solvent peak and clearly visible in MeOD NMR)

¹H NMR (MeOD, 400 MHz): δ ppm, 9.27 (d, J=2.0 Hz, 1H), 8.81 (d, J=2.4 Hz, 1H), 7.99 (dd, J=8.0 Hz, 1H), 7.72-7.65 (m, 2H), 5.87 (s, 1H), 4.45 (s, 2H), 2.79-2.66 (m, 3H), 2.56-2.51 (m, 1H), 2.41-2.34 (m, 1H), 2.17 (d, J=12.8 Hz, 1H), 1.93-1.85 (m, 1H). (Note: —NH proton might exchange with deuterium from MeOD)

LCMS (Method A): 2.438 min, 100%, 254.0 nm, MS: ES+360.07 (M+1)

HPLC (Method A): 8.764 min, 100%, 254.0 nm

Chiral HPLC: Peak 1=3.87 min, 49.62%, 240.0 nm; Peak 2=4.46 min, 50.38%, 240.0 nm

### Example 46—Synthesis of (8-(4-(trifluoromethyl)cyclohex-1-en-1-yl)quinoline-3-carbonyl)-D-alanine (Compound 50)

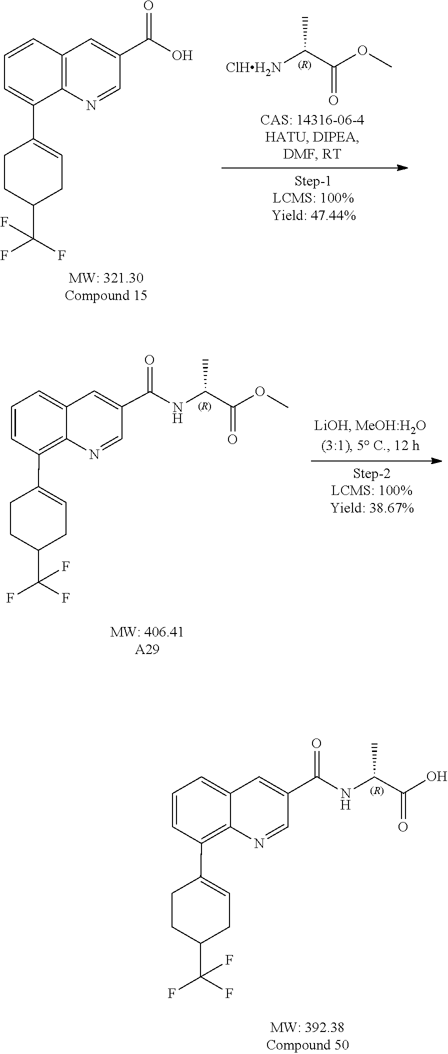

### Step-1:

To a stirred solution of 8-(4-(trifluoromethyl)cyclohex-1-en-1-yl)quinoline-3-carboxylic acid (Compound 15, 0.5 g, 1.5 mmol, 1.0 eq), CAS: 14316-06-4 (0.21 g, 1.54 mmol, 1.0 eq) and DIPEA (0.8 ml, 4.6 mmol, 3.0 eq) in DMF (5 mL) under inert condition N₂(g) was added HATU (0.88 g, 2.3 mmol, 1.5 eq). The reaction was monitored by TLC (using neat ethyl acetate as mobile phase) which confirmed that the reaction got completed after 4 h of stirring at room temperature. The resulting reaction mixture was diluted with water (10 mL) and extracted with EtOAc (3×20 mL). The combined organic layers extracted with EtOAc (2×50 mL), dried over Na₂SO₄ and concentrated under reduced pressure to afford 0.45 g crude. The crude was purified by manual column chromatography using silica (230-400 mesh) as stationary phase (gradient elution 20% Ethyl acetate in hexane) yielding methyl (3R)-3-(8-(4-(trifluoromethyl)cyclohex-1-en-1-yl)quinoline-3-carboxamido) butanoate (A29, 0.3 g, 0.738 mmol, Yield 47.44%).

¹H NMR (DMSO-d₆, 400 MHz): δ ppm 9.26 (d, J=2.0 Hz, 1H), 9.17 (d, J=6.8 Hz, 1H), 8.86 (d, J=2.0 Hz, 1H), 8.02 (dd, J=6.0, 3.6 Hz, 1H), 7.66-7.63 (m, 2H), 5.87 (br s, 1H), 4.59-4.52 (m, 1H), 3.67 (s, 3H), 2.80-2.67 (m, 3H), 2.50-2.46 (m, 1H), 2.32-2.24 (m, 1H), 2.10-2.07 (m, 1H), 1.74-1.64 (m, 1H), 1.45 (d, J=7.6 Hz, 3H)

LCMS: 2.51 min, 100%, 215 nm

### Step-2:

To a stirred solution of A29 (0.15 g, 0.36 mmol, 1.0 eq), LiOH (0.018 g, 0.44 mmol, 1.2 eq) in MeOH:H₂O (8:2) was added at 0° C., then the reaction is allowed to run at room temperature. The reaction was monitored by TLC (using neat ethyl acetate as mobile phase) which confirmed that the reaction got completed after 2 h of stirring at room temperature. The resulting reaction mixture was concentrated then acidified using IN HCl, then filtered using Buchner filtration to yield (8-(4-(trifluoromethyl)cyclohex-1-en-1-yl)quinoline-3-carbonyl)-D-alanine (Compound 50, 0.056 g, 0.143 mmol, Yield 38.67%)

¹H NMR (DMSO, 400 MHz): δ ppm 9.32 (d, J=2.0 Hz, 1H), 9.21 (d, J=6.8 Hz, 1H), 9.04 (s, 1H), 8.08-8.07 (m, 1H), 7.72-7.68 (m, 2H), 5.89 (br s, 1H), 4.53-4.46 (m, 1H), 2.77-2.69 (br s, 2H), 2.65-2.51 (m, 1H), 2.47-2.42 (m, 1H), 2.33-2.26 (m, 1H), 2.10-2.07 (m, 1H), 1.77-1.68 (m, 1H), 1.46 (d, J=7.2 Hz, 3H) (Note: -one CF₃—CH proton merge with DMSO solvent peak and clearly visible in MeOD NMR)

¹H NMR (MeOD, 400 MHz): δ ppm 9.68 (d, J=1.6 Hz, 1H), 9.49 (d, J=1.6 Hz, 1H), 8.38 (d, J=8.0 Hz, 1H), 8.13 (dd, J=6.8 Hz, 1H), 8.05 (t, J=8.0 Hz, 1H), 6.10 (br s, 1H), 4.75-4.70 (m, 1H), 3.42-2.25 (s, 6H), 2.03-1.91 (m, 1H), 1.63 (d, J=7.6 Hz, 3H). (Note: —NH and —OH proton might exchange with deuterium from MeOD)

LCMS: 2.28 min, 100%, 210 nm

HPLC: 4.49 min, 99.17%, 254 nm

CHIRAL HPLC: Peak-1 3.58 min, 48.54%, 240 nm; Peak-2: 3.90 min, 49.73%, 240 nm

### Example 47—Synthesis of O-methyl-N-(8-(4-(trifluoromethyl)cyclohex-1-en-1-yl)quinoline-3-carbonyl)-D-serine (Compound 51)

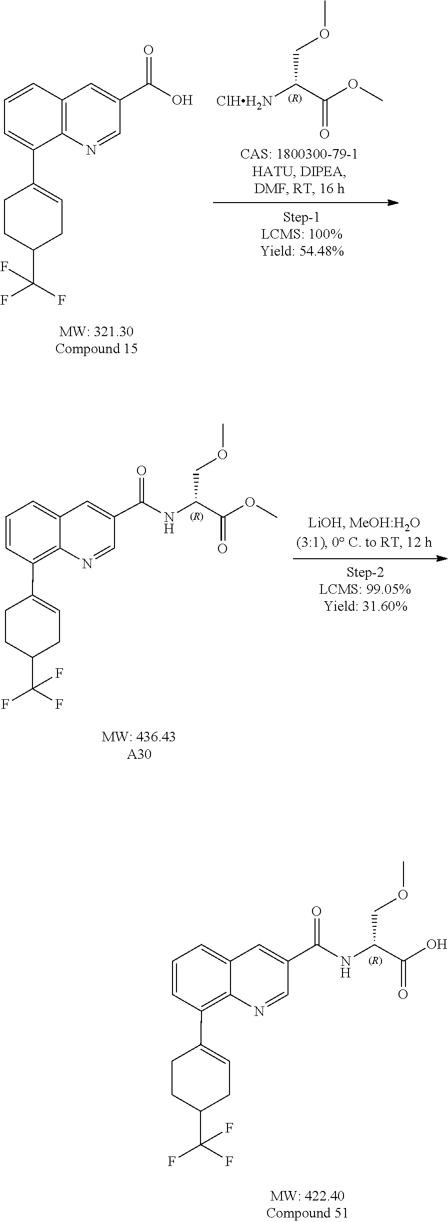

### Step-1:

In 250 mL three neck RB flask, as a white amorphous 8-(4-(trifluoromethyl)cyclohex-1-en-1-yl)quinoline-3-carboxylic acid (Compound 15, 0.250 g, 0.775 mmol)., methyl O-Methyl-D-serinate hydrochloride CAS 1800300-79-1 (0.315 g, 0.934 mmol, 1.2 eq.), and DIPEA (1.12 ml, 2.35 mmol, 3.0 eq.) were stirred with HATU (0.89 g, 1.16 mmol, 1.5 eq.) in DMF (2 mL, 10 v) for 16 h at room temperature. The reaction was monitored on TLC (using EtOAc:Hexane; 3:7 as mobile phase) which confirmed that the reaction got completed after 16 h of stirring at room temperature. The resulting reaction mixture was diluted with ice-cold water (10 mL) and was extracted with DCM. The combined layer was evaporated under vacuum and crude residue was purified on silica column chromatography (60-120 mesh; 30% of EtOAc and hexane) and yielded methyl O-Methyl-N-(8-(4-(trifluoromethyl)cyclohex-1-en-1-yl)quinoline-3-carbonyl)-D-serinate (A30, 0.185 g, 0.424 mmol, Yield: 54.48%).

¹H NMR (DMSO-d₆, 400 MHz): δ ppm 9.25 (d, J=2.0 Hz, 1H), 9.21 (d, J=7.2 Hz, 1H), 8.88 (d, J=4.0 Hz, 1H), 8.03 (dd, J=6.4, 4.8 Hz, 1H), 7.67-7.63 (m, 2H), 5.88 (br s, 1H), 4.82-4.77 (m, 1H), 3.87-3.50 (m, 5H), 3.26 (s, 3H), 2.81-2.68 (m, 4H), 2.33-2.25 (m, 1H), 2.30-2.20 (m, 1H), 2.10-2.1 (m, 1H), 1.74-1.64 (m, 1H).

LCMS (Method A): 2.513 min, 100.0%, MS: ES+437.18 [M+H]

### Step-2:

A suspension of methyl O-Methyl-N-(8-(4-(trifluoromethyl)cyclohex-1-en-1-yl)quinoline-3-carbonyl)-D-serinate (A30, 0.170 g, 0.3895 mmol 1.0 eq) in THF/water (3 mL/1 mL) was cooled to 0° C., then treated with LiOH (0.033 g, 0.824 mmol 2.0 eq), and the reaction solution stirred at this temperature for 12 hours. The solution was acidified with 1N HCl. The resulting reaction mixture was diluted with water (10 mL) and extracted with EtOAc (3×20 mL). The combined organic layers extracted with EtOAc (2×50 mL), washed dried to give as gray solid O-methyl-N-(8-(4-(trifluoromethyl)cyclohex-1-en-1-yl)quinoline-3-carbonyl)-D-serine (Compound 51, 0.052 g, 0.123 mmol 31.06% yield).

¹H NMR (400 MHz, DMSO-d₆) δ ppm: 9.27 (d, J=2.0 Hz, 1H), 9.11 (d, J=7.6 Hz, 1H,) 8.93 (s, 1H), 8.03 (dd, J=6.4, 4.8 Hz, 1H), 7.67-7.64 (m, 2H), 5.88 (br s, 1H), 4.72 (q, J=10.0 Hz, 1H), 3.83-3.68 (m, 1H), 3.32 (s, 3H), 2.81-2.67 (m, 3H), 2.58-2.51 (m, 1H), 2.33-2.26 (m, 1H), 2.10-2.07 (m, 1H), 1.76-1.66 (m, 1H) (Note: -one CF₃—CH proton merge with DMSO solvent peak and clearly visible in MeOD NMR)

¹H NMR (400 MHz, MeOD) δ (ppm): 9.48 (s, 1H), 9.42 (d, J=2.0 Hz, 1H), 8.29 (d, J=4.0 Hz, 1H), 8.02 (d, J=4.0 Hz, 1H), 7.96 (t, J=8.0 Hz, 1H), 6.04 (br s, 1H), 4.00-3.97 (m, 1H), 3.89-3.82 (m, 1H), 3.45 (s, 3H) 3.74 (br s, 2H), 2.65-2.56 (m, 2H), 2.48-2.41 (m, 1H), 2.61-2.24 (m, 1H), 2.01-1.90 (m, 1H) (Note: —NH and —OH proton might exchange with deuterium from MeOD)

LCMS (Method A): 2.31 min, 95.47%, 254 nm

HPLC (Method A): 4.34 min, 96.33%, 254 nm

CHIRAL HPLC: Peak-1 6.26 min, 36.05%, 240 nm: Peak-2 6.71 min, 43.16%, 240 nm

### Example 48—Synthesis of N—((R)-1-(methylamino)-1-oxopropan-2-yl)-8-(4 (trifluoromethyl)cyclohex-1-en-1-yl)quinoline-3-carboxamide (Compound 52)

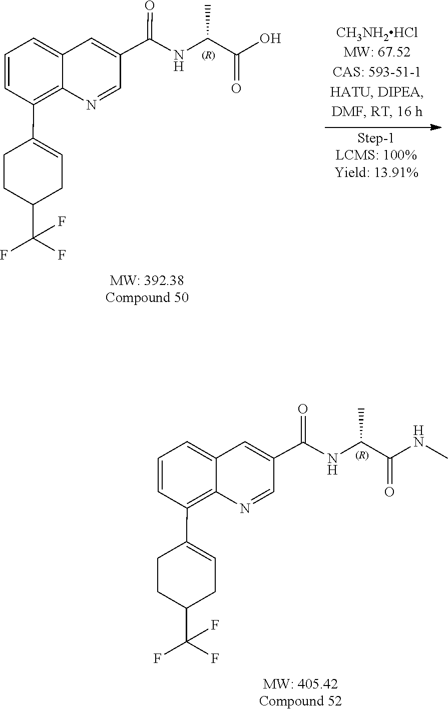

### Step-1:

A stirred solution of 8-(4-(trifluoromethyl)cyclohex-1-en-1-yl)quinoline-3-carboxylic acid (Compound 50, 0.16 g, 0.40 mmol, 1.0 eq), CAS: 61302-99-6 (0.027 g, 0.40 mmol, 1.0 eq) and DIPEA (0.2 ml, 1.2 mmol, 3.0 eq) in DMF (2 mL) was prepared under inert condition N₂(g) then HATU (0.232 g, 0.612 mmol, 1.5 eq) was added. The reaction was monitored by TLC (using neat ethyl acetate as mobile phase) which confirmed that the reaction got completed after 16 h of stirring at room temperature. The resulting reaction mixture was diluted with water (10 mL) and extracted with EtOAc (3×20 mL). The combined organic layers extracted with EtOAc (2×50 mL), dried over Na₂SO₄ and concentrated under reduced pressure to afford 0.45 g crude. The crude was purified by manual column chromatography using silica (230-400 mesh) as stationary phase (gradient elution 20% Ethyl acetate in hexane) then purified by prep HPLC purification (Method-A) yielding N—((R)-1-(methylamino)-1-oxopropan-2-yl)-8-(4-(trifluoromethyl)cyclohex-1-en-1-yl)quinoline-3-carboxamide (Compound 52, 0.023 g, 0.057 mmol, Yield 13.91%).

¹H NMR (DMSO-d₆, 400 MHz): δ ppm 9.28 (d, J=2.0 Hz, 1H), 8.91-8.88 (m, 2H), 8.02-7.80 (m, 2H), 7.95 (m, J=4.4 Hz, 2H), 7.65-7.64 (m, 1H), 5.88 (br s, 1H), 4.51-4.48 (m, 1H), 2.79 (br s, 1H), 2.72-2.68 (m, 2H), 2.62-2.57 (m, 4H), 2.33-2.29 (m, 1H), 2.10-2.07 (m, 1H), 1.72-1.68 (m, 1H), 1.37 (d, J=7.2 Hz, 3H) (Note: -one CF₃—CH proton merge with DMSO solvent peak and clearly visible in MeOD NMR)

¹H NMR (MeOD, 400 MHz): δ ppm 9.28 (d, J=2.0 Hz, 1H), 8.85 (d, J=2.0 Hz, 1H), 7.98 (dd, J=7.8, 1.6 Hz, 1H), 7.70-7.64 (m, 2H), 5.87 (br s, 1H), 4.61 (q, J=7.2 Hz, 1H), 2.80 (s, 3H), 2.79-2.75 (m, 2H), 2.68-2.65 (m, 1H), 2.56-2.51 (m, 1H), 2.41-2.38 (m, 1H), 2.18-2.15 (m, 1H), 1.53 (d, J=7.2 Hz, 3H) (Note: —NH proton might exchange with deuterium from MeOD)

LCMS (Method A): 2.20 min, 100%, 254.0 nm, m/z=406.12 (M+H)+

HPLC (Method A): 7.75 min, 99.61%, 210 nm

CHIRAL HPLC: Peak-1: 3.55 min, 42.28%, 245 nm: Peak-2: 4.07 min, 43.73%, 245 nm

### Example 49—Synthesis of N—((R)-1-aminopropan-2-yl)-8-(4-(trifluoromethyl)cyclohex-1-en-1-yl)quinoline-3-carboxamide (Compound 53)

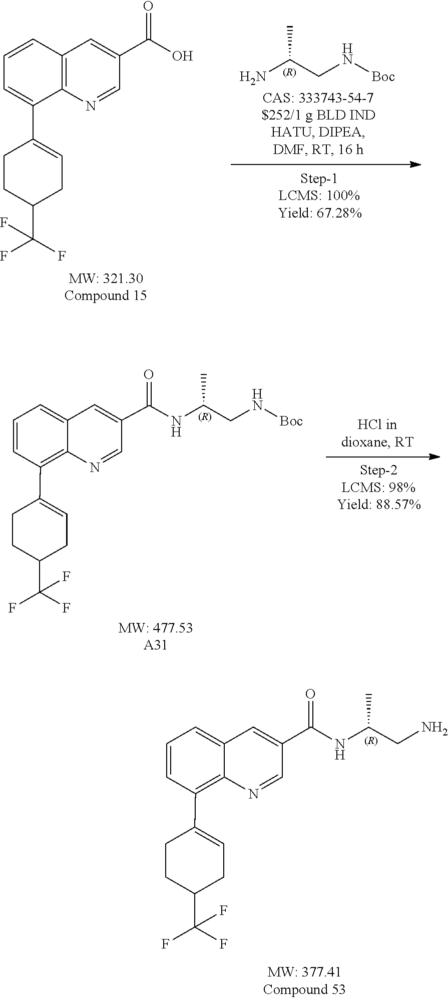

### Step-1:

To a stirred solution of 8-(4-(trifluoromethyl)cyclohex-1-en-1-yl)quinoline-3-carboxylic acid (Compound 15, 0.2 g, 0.62 mmol, 1.0 eq), CAS: 333743-54-7 (0.10 g, 0.62 mmol, 1.0 eq) and DIPEA (0.3 ml, 1.8 mmol, 3.0 eq) in DMF (2 mL) under inert condition N₂(g) was added HATU (0.35 g, 0.93 mmol, 1.5 eq). The reaction was monitored by TLC (using neat ethyl acetate as mobile phase) which confirmed that the reaction got completed after 4 h of stirring at room temperature. The resulting reaction mixture was diluted with water (10 mL) and extracted with EtOAc (3×20 mL). The combined organic layers extracted with EtOAc (2×50 mL), dried over Na₂SO₄ and concentrated under reduced pressure to afford 0.3 g crude. The crude was purified by manual column chromatography using silica (230-400 mesh) as stationary phase (gradient elution 20% Ethyl acetate in hexane) yielding tert-butyl ((2R)-2-(8-(4-(trifluoromethyl)cyclohex-1-en-1-yl)quinoline-3-carboxamido)propyl)carbamate (A31, 0.22 g, 0.461 mmol, Yield 67.28%).

¹H NMR (DMSO-d₆, 400 MHz): δ ppm 9.23 (d, J=1.6 Hz, 1H), 8.77 (d, J=1.6 Hz, 1H), 8.48 (d, J=8.4 Hz, 1H), 7.98 (t, J=4.8 Hz, 1H), 7.64-7.61 (m, 2H), 7.01 (t, J=5.6 Hz, 1H), 5.87 (br s, 1H), 4.15-4.08 (m, 1H), 3.43-3.07 (m, 2H), 2.79-2.68 (m, 4H), 2.31-2.25 (m, 1H), 2.01-2.07 (m, 1H), 1.74-1.63 (m, 1H), 1.35 (s, 9H), 1.14 (d, J=6.8 Hz, 3H)

LCMS (Method-A): RT=2.72 min, 100%, 254 nm, m/z=478.24 (M+H)+

### Step-2:

To a stirred solution of A31 (0.1 g, 0.20 mmol, 1.0 eq), in DCM (1 ml) was added 4M HCl in Dioxane at 0° C., then the reaction is allowed to run at room temperature. The reaction was monitored by TLC (using neat ethyl acetate as mobile phase) which confirmed that the reaction got completed after 16 h of stirring at room temperature. The resulting reaction mixture was concentrated then triturated using pentane and diethyl ether to yield N—((R)-1-aminopropan-2-yl)-8-(4-(trifluoromethyl)cyclohex-1-en-1-yl)quinoline-3-carboxamide (Compound 53, 0.070 g, 0.18 mmol, Yield 88.57%)

¹H NMR (DMSO-d₆, 400 MHz): δ ppm 9.33 (s, 1H), 8.96 (s, 1H), 8.90 (d, J=8.0 Hz, 1H), 8.09 (br s, 2H), 8.04-8.02 (m, 1H), 7.68-7.66 (m, 2H), 5.88 (br s, 1H), 4.35 (t, J=6.8 Hz, 1H), 3.01 (br s, 2H), 2.76-2.67 (m, 3H), 2.32-2.25 (m, 1H), 2.10-2.07 (m, 1H), 1.72-1.68 (m, 1H), 1.23 (d, J=6.4 Hz, 3H). (Note: -one CF₃—CH proton merge with DMSO solvent peak and clearly visible in MeOD NMR)

¹H NMR (MeOD, 400 MHz): δ ppm 9.60 (s, 1H), 9.49 (s, 1H), 8.34 (d, J=8.0 Hz, 1H), 8.08 (d, J=6.4 Hz, 1H), 8.01 (t, J=7.6 Hz, 1H), 6.07 (br s, 1H), 4.56-4.52 (m, 1H), 3.33-3.16 (m, 2H), 2.75-2.61 (m, 4H), 2.49-2.42 (m, 1H), 2.28-2.25 (m, 1H), 2.0-1.90 (m, 1H), 1.46 (d, J=6.8 Hz, 3H) (Note: —NH proton might exchange with deuterium from MeOD)

LCMS (Method-A): 1.86 min, 100%, 210.0 nm, m/z=378.1

HPLC (Method-A): 8.34 min, 100%, 210 nm

CHIRAL HPLC: Peak-1: 5.27 min, 47.54%, 240 nm; Peak-2: 5.75 min, 52.45%, 240 nm

### Example 50—Synthesis of N—((R)-4-hydroxybutan-2-yl)-8-(4-(trifluoromethyl)cyclohex-1-en-1-yl)quinoline-3-carboxamide (Compound 54)

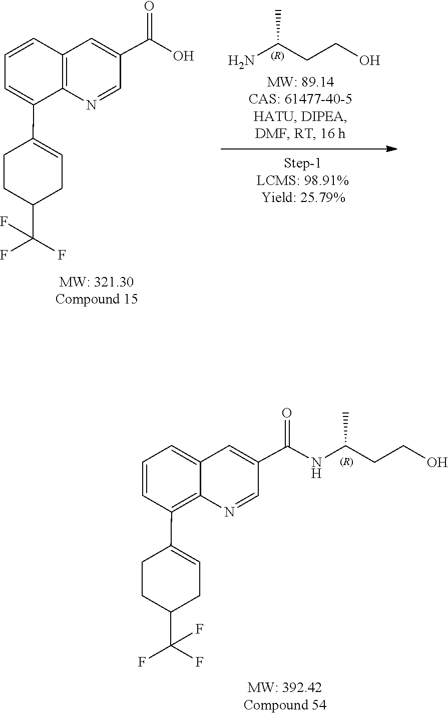

### Step-1:

To a stirred solution of 8-(4-(trifluoromethyl)cyclohex-1-en-1-yl)quinoline-3-carboxylic acid (Compound 15, 0.2 g, 0.62 mmol, 1.0 eq), CAS: 61477-40-5 (0.055 g, 062 mmol, 1.0 eq) and DIPEA (0.3 ml, 1.8 mmol, 3.0 eq) in DMF (2 mL) under inert condition N₂(g) was added HATU (0.355 g, 0.934 mmol, 1.5 eq). The reaction was monitored by TLC (using neat ethyl acetate as mobile phase) which confirmed that the reaction got completed after 4 h of stirring at room temperature. The resulting reaction mixture was diluted with water (10 mL) and extracted with EtOAc (3×20 mL). The combined organic layers extracted with EtOAc (2×50 mL), dried over Na₂SO₄ and concentrated under reduced pressure to afford 0.45 g crude. The crude was purified by manual column chromatography using silica (230-400 mesh size) as stationary phase (gradient elution 20% Ethyl acetate in hexane) yielding N—((R)-4-hydroxybutan-2-yl)-8-(4-(trifluoromethyl)cyclohex-1-en-1-yl)quinoline-3-carboxamide (Compound 54, 0.063 g, 0.161 mmol, Yield 25.79%).

¹H NMR (DMSO-d₆, 400 MHz): δ ppm 9.23 (d, J=1.6 Hz, 1H), 8.78 (d, J=2.0 Hz, 1H), 8.55 (d, J=8.4 Hz, 1H), 8.00 (t, J=4.8 Hz, 1H), 7.64 (d, J=4.4 Hz, 2H), 5.87 (br s, 1H), 4.47 (t, J=4.8 Hz, 1H), 4.21-4.14 (m, 1H), 3.49-3.46 (m, 2H), 2.80-2.07 (m, 5H), 1.80-1.42 (m, 3H), 1.20 (d, J=6.4 Hz, 3H) (Note: -one CF₃—CH proton merge with DMSO solvent peak and clearly visible in MeOD NMR)

¹H NMR (MeOD, 400 MHz): δ ppm 9.23 (d, J=2.0 Hz, 1H), 8.75 (d, J=2.0 Hz, 1H), 7.97 (d, J=6.4 Hz, 1H), 7.69-7.63 (m, 2H), 6.87 (br s, 1H), 4.39-4.34 (m, 1H), 3.75-3.66 (m, 2H), 2.88-2.65 (m, 3H), 2.55-2.51 (m, 1H), 2.40-2.34 (m, 1H), 2.18-2.15 (m, 1H), 1.91-1.82 (m, 3H), 1.35 (d, J=6.8 Hz, 3H). (Note: —NH proton might exchange with deuterium from MeOD)

LCMS (Method-A): 2.27 min, 98.91%, 254.0 nm, m/z=393.17 (m+H)⁺

HPLC (Method-A): 8.25 min, 100%, 210 nm

CHIRAL HPLC: Peak-1: 4.22 min, 50.06%, 240 nm; Peak-2: 4.53 min, 49.93%, 240 nm

### Example 51—Synthesis of (3R)-3-(8-(4-(trifluoromethyl)cyclohex-1-en-1-yl)quinoline-3-carboxamido)butanoic Acid (Compound 55)

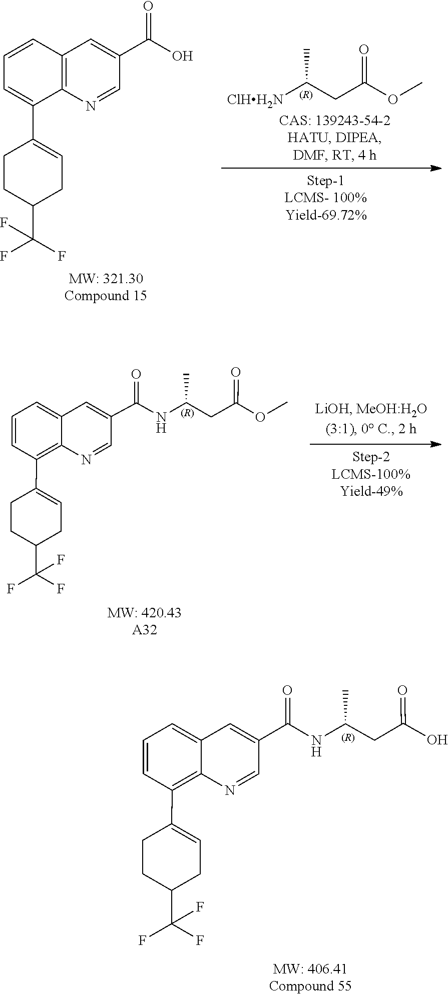

### Step-1

To a stirred solution of 8-(4-(trifluoromethyl)cyclohex-1-en-1-yl)quinoline-3-carboxylic acid (Compound 15, 0.45 g, 1.4 mmol, 1.0 eq), CAS: 139243-54-2 (0.23 g, 1.54 mmol, 1.1 eq) and DIPEA (0.542 g, 4.2 mmol, 3.0 eq) in DMF (2 mL) under inert condition N₂(g) was added HATU (0.798 g, 2.1 mmol, 1.5 eq). The reaction was monitored by TLC (using neat ethyl acetate as mobile phase) which confirmed that the reaction got completed after 4 h of stirring at room temperature. The resulting reaction mixture was diluted with water (10 mL) and extracted with EtOAc (3×20 mL). The combined organic layers extracted with EtOAc (2×50 mL), dried over Na₂SO₄ and concentrated under reduced pressure to afford 0.45 g crude. The crude was purified by manual column chromatography using silica (230-400 mesh) as stationary phase (gradient elution 20% Ethyl acetate in hexane) yielding methyl (3R)-3-(8-(4-(trifluoromethyl)cyclohex-1-en-1-yl)quinoline-3-carboxamido) butanoate (A32, 0.41 g, 0.975 mmol, Yield 69.72%)

¹H NMR (MeOD, 400 MHz): δ ppm 9.22 (d, J=2.4 Hz, 1H), 8.73 (d, J=2.0 Hz, 1H), 7.96 (dd, J=7.6, 1.6 1H), 7.69-7.63 (m, 2H), 5.86 (br s, 1H), 4.62-4.57 (m, 1H), 3.70 (s, 3H), 2.77-2.72 (m, 2H), 2.68-2.63 (m, 3H), 2.55-2.51 (m, 1H), 2.41-2.37 (m, 1H), 2.18-2.15 (m, 1H), 1.90-1.85 (m, 1H), 1.37 (d, J=6.8 Hz, 3H). (Note: —NH proton might exchange with deuterium from MeOD; EA solvent Traces observed)

LCMS (Method A): 2.523 min, 100%, 254 nm, (M+H)⁺=421.12

CHIRAL HPLC: Peak-1 3.31 min, 49.60%, 240 nm Peak-2 3.52 min, 50.39%, 240 nm

### Step-2

To a stirred solution of (A32, 0.38 g, 0.90 mmol, 1.0 eq), LiOH·H₂O (0.094 g, 1.80 mmol, 2.0 eq) in MeOH:H₂O (8:2) was added at 0° C., then the reaction is allowed to run at room temperature. The reaction was monitored by TLC (using neat ethyl acetate as mobile phase) which confirmed that the reaction got completed after 2 h of stirring at room temperature. The resulting reaction mixture was concentrated then acidified using 1N HCl, then filtered using Buchner filtration to yield (3R)-3-(8-(4-(trifluoromethyl)cyclohex-1-en-1-yl)quinoline-3-carboxamido) butanoic acid (Compound 55, 0.18 g, 0.44 mmol, Yield 49%)

¹H NMR (DMSO-d₆, 400 MHz): δ ppm 9.24 (d, J=2.0 Hz, 1H), 8.84 (s, 1H), 8.75 (d, J=7.6 Hz, 1H), 8.04-8.02 (m, 1H), 7.68-7.66 (m, 3H), 5.87 (br s, 1H), 4.43-4.35 (m, 1H), 2.79-2.61 (m, 4H), 2.47-2.43 (m, 1H), 2.33-2.29 (m, 1H), 2.10-2.07 (m, 1H), 1.73-1.68 (m, 1H), 1.24 (d, J=8.0 Hz, 3H) (Note: -one CF₃—CH proton merge with DMSO solvent peak and clearly visible in MeOD NMR)

¹H NMR (MeOD, 400 MHz): δ ppm 9.52 (d, J=1.6 Hz, 1H), 9.41 (d, J=1.6 Hz, 1H), 8.32 (d, J=7.6 Hz, 1H), 8.08 (d, J=6.0 Hz, 1H), 8.01 (t, J=8.0 Hz, 1H), 6.07 (br s, 1H), 4.64-4.59 (m, 1H), 2.80-2.74 (m, 4H), 2.66-2.63 (m, 2H), 2.49-2.46 (m, 1H), 2.28-2.25 (m, 1H), 1.97-1.93 (m, 1H), 1.41 (d, J=6.8 Hz, 3H) (Note: —NH and —OH proton might exchange with deuterium from MeOD)

LCMS (Method A): 2.28 min, 100%, 242.0 nm, (M+H)⁺=407.12

HPLC (Method A): 4.51 min, 99.47%, 254 nm

CHIRAL HPLC: Peak-1 4.97 min, 47.33%, 240 nm; Peak-2 5.52 min, 44.05%, 240 nm

### Example 52—Synthesis of N—((R)-4-(methylamino)-4-oxobutan-2-yl)-8-(4-(trifluoromethyl)cyclohex-1-en-1-yl)quinoline-3-carboxamide (Compound 56)

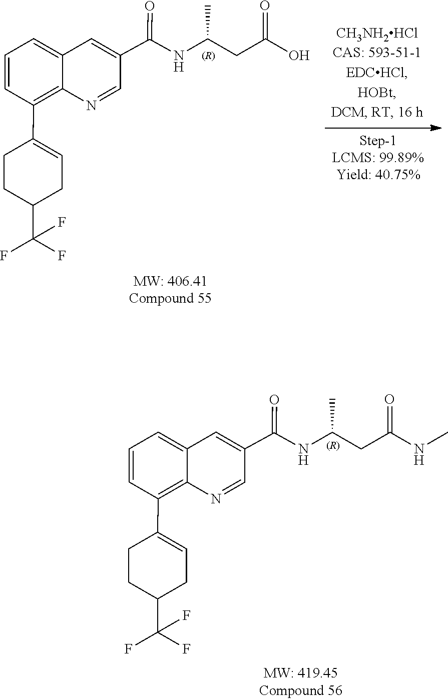

### Step-1

To a stirred solution of (3R)-3-(8-(4-(trifluoromethyl)cyclohex-1-en-1-yl)quinoline-3-carboxamido)butanoic acid (Compound 55, 0.107 g, 0.263 mmol, 1.0 eq), CAS: 593-51-1 (0.0195 g, 0.289 mmol, 1.1 eq) and DIPEA (0.102 g, 0.789 mmol, 3.2 eq) in DCM (2 mL) under inert condition N₂ (g) were added EDCl·HCl (0.0757 g, 0.395 mmol, 1.5 eq) and HOBt (0.0534 g, 0.39 mmol, 1.5 eq). The reaction was monitored by TLC (using neat ethyl acetate as mobile phase) which confirmed that the reaction got completed after 16 h of stirring at room temperature. The resulting reaction mixture was diluted with water (10 mL) and extracted with EtOAc (3×20 mL). The combined organic layers extracted with EtOAc (2×50 mL), dried over Na₂SO₄ and concentrated under reduced pressure to afford 0.07 g crude. The crude was purified by manual column chromatography using silica (230-400 mesh) as stationary phase (gradient elution in neat ethyl acetate) yielding N—((R)-4-(methylamino)-4-oxobutan-2-yl)-8-(4-(trifluoromethyl)cyclohex-1-en-1-yl)quinoline-3-carboxamide (Compound 56, 0.045 g, 0.107 mmol, Yield 40.75%)

¹H NMR (MeOD, 400 MHz): δ ppm 9.22 (d, J=2.0 Hz, 1H), 8.74 (d, J=2.0 Hz, 1H), 7.97 (dd, J=7.4, 2.0 Hz, 1H), 7.69-7.63 (m, 2H), 5.87 (br s, 1H), 4.59-4.54 (m, 1H), 2.89-2.75 (m, 4H), 2.68-2.65 (m, 1H), 2.62-2.61 (m, 1H), 2.59-2.46 (m, 3H), 2.41-2.34 (m, 1H), 2.41-2.34 (m, 1H), 1.93-1.83 (m, 1H), 1.38 (d, (d, J=6.8 Hz, 3H)

¹H NMR (CD₃CN, 400 MHz): δ ppm 9.20 (d, J=2.0 Hz, 1H), 8.63 (d, J=2.0 Hz, 1H), 7.94 (dd, J=7.6, 1.6 Hz, 1H), 7.71 (d, J=7.6 Hz, 1H), 7.66-7.60 (m, 2H), 6.51 (br s, 1H), 5.89 (br s, 1H), 4.51-4.45 (m, 1H), 2.83-2.48 (m, 2H), 2.43 (d, J=6.4 Hz, 3H), 2.40-2.32 (m, 4H), 1.83-1.77 (m, 2H), 1.96-1.78 (m, 1H), 1.29 (d, J=6.8 Hz, 3H)

LCMS (Method A): 2.22 min, 99.89%, 254 nm, (M+H)⁺=420.18

HPLC (Method A): 7.69 min, 99.53%, 210 nm

CHIRAL HPLC: Peak-1 11.21 min, 52.16%, 225 nm; Peak-2 11.81 min, 47.84%, 225 nm

### Example 53—Synthesis of 5-(4,4-difluorocyclohex-1-en-1-yl)-N-isopropyl-2-naphthamide (Compound 57)

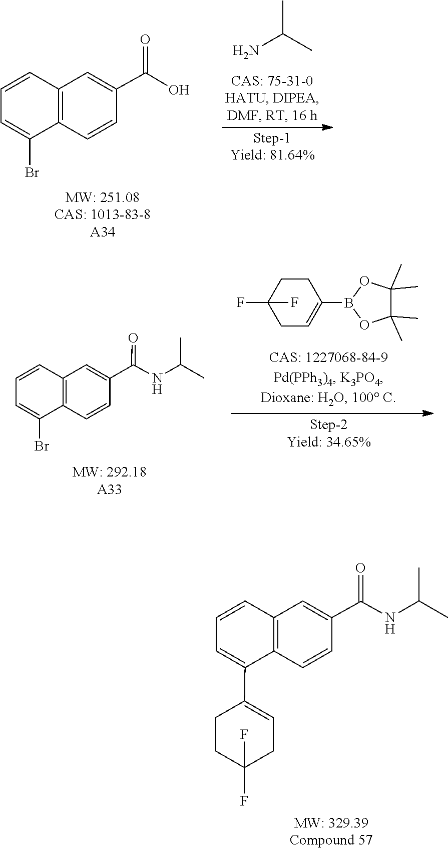

### Step-1

To a solution of 5-bromo-2-naphthoic acid CAS: 1013-83-8 (1.0 g, 3.982 mmol, 1.0 eq), HATU (2.27 g, 5.974 mmol, 1.5 eq) and DIPEA (1.36 mL, 7.965 mmol, 2.0 eq) in DCM (10 mL) at 0° C. under nitrogen atmosphere stirred for 20 min, was added propan-2-amine CAS: 75-31-0 (0.258 g, 4.389 mmol, 1.1 eq) under Nitrogen. The resulting mixture was stirred for 16 h at room temperature. The reaction was monitored on TLC (using EtOAc:Hexane; 3:7 as mobile phase) which confirmed that the reaction got completed after 16 h. The resulting reaction mixture was quenched with ice-cold water (20 mL) and extracted with DCM (3×25 mL). The combined organic layers were dried over Na₂SO₄ and concentrated under reduced pressure to afford 1.3 g crude. The obtained crude material was purified by flash column chromatography using silica (230-400 mesh) as stationary phase (0-20% Ethyl acetate in hexane) yielding 5-bromo-N-isopropyl-2-naphthamide (A33) (0.95 g, 3.251 mmol, Yield: 81.64%).

¹H NMR (DMSO-d⁶, 400 MHz): δ ppm, 8.51-8.48 (m, 2H), 8.17 (d, J=8.8 Hz, 1H), 8.09 (dd, J=8.8, 1.6 Hz, 2H), 7.98 (d, J=7.6 Hz, 1H), 7.52 (t, J=8.0 Hz, 1H), 4.19-4.14 (m, 1H), 1.27-1.17 (m, 6H). Note: Some aliphatic impurities present that related to SM.

LCMS (Method A): 2.286 min, 97.67%, 254.0 nm, MS: ES+292.1 (M), 294.1 (M+2)

### Step-2

A stirred solution of 5-bromo-N-isopropyl-2-naphthamide (A33) (0.3 g, 1.229 mmol, 1.0 eq.), 2-(4,4-difluorocyclohex-1-en-1-yl)-4,4,5,5-tetramethyl-1,3,2-dioxaborolane, CAS: 1227068-84-9 (Purchased from commercial source combi block) (0.32 g, 1.106 mmol, 0.9 eq), and Tripotassium phosphate (0.78 g, 3.687 mmol, 3.0 eq) in 1,4-dioxane (3 mL) was purged with nitrogen for 30 minutes then Pd(PPh₃)₄ (0.143 g, 0.123 mmol, 1.3 eq.) was added and the resulting reaction mixture was heated up to 100° C. for 16 h. The reaction was monitored by TLC (using EA:Hex; 3:7 as mobile phase) which confirmed that the reaction got completed after 16 h of stirring at 100° C. The resulting reaction mixture was filtered through celite bed and the filtrate was concentrated under reduced pressure to obtained the crude residue (0.4 g) which was purified by silica gel (60-120 mesh) as stationary phase (25% EtOAc in Hex) yielded 5-(4,4-difluorocyclohex-1-en-1-yl)-N-isopropyl-2-naphthamide (Compound 57) (0.14 g, 0.425 mmol, Yield: 34.65%).

¹H NMR (DMSO-d6, 400 MHz): δ ppm 8.45 (s, 1H), 8.39 (d, J=7.6 Hz, 1H), 7.97-7.91 (m, 3H), 7.55 (t, J=8.0 Hz, 1H), 7.40 (d, J=6.8 Hz, 1H), 5.64 (br s, 1H), 4.18-4.13 (m, 1H), 2.82 (t, J=14.8 Hz, 2H), 2.65-2.55 (m, 2H), 2.34-2.24 (m, 2H), 1.21 (d, J=6.4 Hz, 6H).

LCMS (Method A): 2.398 min, 98.32%, MS: ES+330.16 [M+H]

HPLC (Method C): 8.357 min, 96.83%, 210 nm

### Example 54—Synthesis of N—((S)-1-(pyridin-2-yl)ethyl)-5-(4-(trifluoromethyl)cyclohex-1-en-1-yl)-2-naphthamide (Compound 58)

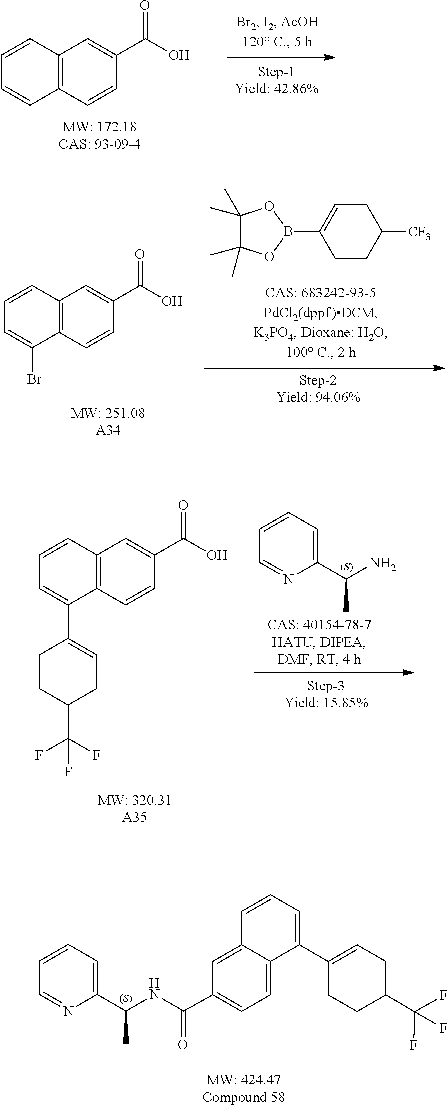

### Step-1

A solution of 2-naphthoic acid CAS: 93-09-4 (24.0 g, 139.4 mmol, 1.0 eq), Br₂ (22.2 g, 139.4 mmol, 1.0 eq) and I₂ (0.706 g, 2.788 mmol, 0.02 eq) in AcOH (240 mL) was stirred at 120° C. for 5 h. The reaction was monitored on TLC (using EA:Hex; 2.0:8.0 as mobile phase) which confirmed the completion of reaction after 5 h of stirring at 120° C. The resulting reaction mixture was cool down to RT. The solid obtained was filtered off and washed with AcOH (50 mL) and water (200 mL) to afford 20.0 g crude product. Then the crude material was triturated by using MeOH (2×50 mL) to afford 5-bromo-2-naphthoic acid (A34) (15.0 g, 59.74 mmol, Yield: 42.86%)

¹H NMR (DMSO-d₆, 400 MHz): δ ppm, 13.31 (br s, 1H), 8.68 (d, J=1.6 Hz, 1H), 8.21 (t, J=8.4 Hz, 2H), 8.14 (dd, J=8.8, 1.6 Hz, 1H), 8.03 (dd, J=7.6, 1.2 Hz, 1H), 7.54 (t, J=7.6 Hz, 1H).

### Step-2

To a stirred solution of 5-bromo-2-naphthoic acid (A34) (0.25 g, 0.996 mmol, 1.0 eq), 4,4,5,5-tetramethyl-2-(4-(trifluoromethyl)cyclohex-1-en-1-yl)-1,3,2-dioxaborolane (CAS: 683242-93-5) (0.41 g, 1.195 mmol, 1.2 eq) and K₃PO₄ (1.05 g, 4.980 mmol, 5.0 eq) in Dioxane (2 mL) and water (1 mL) was added Pd(dppf)Cl₂: DCM (0.081 g, 0.099 mmol, 0.1 eq) under N₂. The resulting mixture was stirred at 100° C. for 2 h. The reaction was monitored by TLC (using EA:Hex; 8.0:2.0 as mobile phase) which confirmed that the reaction got completed after 2 h of stirring at 100° C. The resulting reaction mixture was diluted with water (10 mL) and extracted with EtOAc (3×20 mL). The combined organic layers were dried over Na₂SO₄ and concentrated under reduced pressure to afford 0.320 g crude. The obtained crude material was purified by flash column chromatography using silica gel (230-400 mesh) as stationary phase (69% EA in Hexane) to afford 5-(4-(trifluoromethyl)cyclohex-1-en-1-yl)-2-naphthoic acid (A35) (0.30 g, 0.936 mmol, Yield: 94.06%).

¹H NMR (DMSO-d6, 400 MHz): δ ppm, 13.11 (br s, 1H), 8.60 (d, J=1.6 Hz, 1H), 8.06 (t, J=11.2 Hz, 2H), 7.96 (dd, J=8.8, 2.0 Hz, 1H), 7.57 (t, J=8.0 Hz, 1H), 7.42 (dd, J=7.2, 1.2 Hz, 1H), 5.73 (br s, 1H), 3.94 (br s, 1H), 2.80-2.77 (m, 1H), 2.56-2.54 (m, 1H), 2.49-2.27 (m, 2H), 2.10-2.07 (m, 1H), 1.77-1.73 (m, 1H). Note: Minor aliphatic impurity peaks were observed.

LCMS (Method A): 2.732 min, 92.64%, 280.0 nm, MS: ES-319.4 (M−1)

### Step-3

To a solution of 5-(4-(trifluoromethyl)cyclohex-1-en-1-yl)-2-naphthoic acid (A35) (0.15 g, 0.468 mmol, 1.0 eq), HATU (0.26 g, 0.703 mmol, 1.5 eq) and DIPEA (0.17 g, 1.370 mmol, 3.0 eq) in DMF (2 mL) at 0° C. under nitrogen atmosphere stirred for 15 min, was added CAS: 40154-78-7 (0.068 g, 0.562 mmol, 1.2 eq) under N₂ atmosphere. The resulting mixture was stirred for 4 h at room temperature. The reaction was monitored by TLC (using EA:Hexane; 5.0:5.0 as mobile phase) which confirmed that the reaction got completed after 4 h. The resulting reaction mixture was quenched with ice-cold water (10 mL) and extracted with EtOAc (3×15 mL). The combined organic layers were dried over Na₂SO₄ and concentrated under reduced pressure to afford 0.250 g crude. The obtained crude material was purified by flash column chromatography using silica (230-400 mess) as stationary phase (35% Ethyl acetate in hexane) to afford N—((S)-1-(pyridin-2-yl)ethyl)-5-(4-(trifluoromethyl)cyclohex-1-en-1-yl)-2 naphthamide (Compound 58) (0.063 g, 0.148 mmol, Yield: 15.85%)

¹H NMR (DMSO, 400 MHz): δ ppm, 9.01 (d, J=8.0 Hz, 1H), 8.56-8.53 (m, 2H), 8.04 (d, J=9.2 Hz, 1H), 7.96-7.94 (m, 2H), 7.76 (td, J=8.0, 1.6 Hz, 1H), 7.56 (t, J=8.0 Hz, 1H), 7.46-7.38 (m, 2H), 7.28-7.25 (m, 1H), 5.74 (br s, 1H), 5.28-5.24 (m, 1H), 2.79 (br s, 1H), 2.52-2.51 (m, 1H), 2.45-2.33 (m, 3H), 2.11-2.08 (m, 1H), 1.83-1.73 (m, 1H), 1.55 (d, J=7.2 Hz, 3H).

¹H NMR (MeOD, 400 MHz): δ ppm, 8.56-8.54 (m, 1H), 8.47 (d, J=1.6 Hz, 1H), 8.06 (d, J=8.8 Hz, 1H), 7.94-7.92 (m, 2H), 7.85 (td, J=7.6, 2.0 Hz, 1H), 7.56-7.52 (m, 2H), 7.40 (dd, J=6.8, 1.2 Hz, 1H), 7.35-7.31 (m, 1H), 5.79 (br s, 1H), 5.35 (q, J=7.2 Hz, 1H), 2.67-2.38 (m, 5H), 2.22-2.18 (m, 1H), 1.89-1.85 (m, 1H) 1.65 (d, J=7.2 Hz, 3H). Note: Amidic-NH not observed.

LCMS (Method A): 2.572 min, 97.37%, 210.0 nm, MS: ES+425.23 (M+1)

HPLC (Method B): 6.907 min, 95.34%, 210.0 nm

Chiral HPLC (Method A): 2.51 min, 95.16%, 230.0 nm

### Example 55—Synthesis of N-isopropyl-8-(spiro[2.5]oct-5-en-6-yl)quinoline-3-carboxamide (Compound 59)

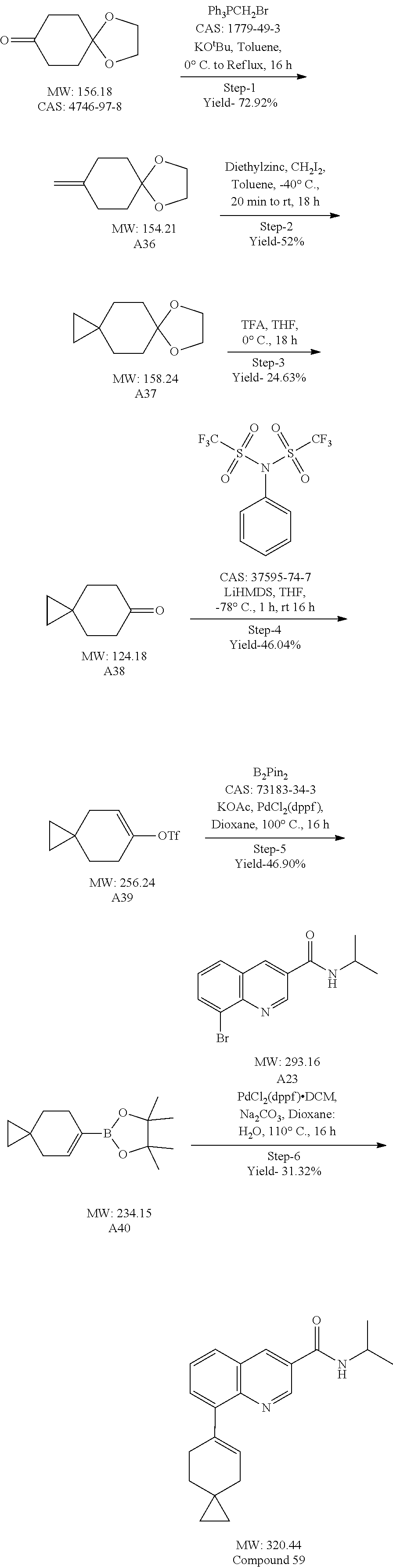

### Step-1

To a stirred solution of 1,4-dioxaspiro[4.5]decan-8-one (CAS: 4746-97-8) (25 g, 160.07 mmol, 1.0 eq), CAS: 1779-49-3 (57.1 g, 160.07 mmol, 1.0 eq) in toluene (200 mL) was added KO^{t}Bu (21.5 g, 192.08 mmol, 1.2 eq) to the reaction mixture under N₂ atmosphere. Then the resulting mixture was stirred at room temperature 110° C. for 16 h. The reaction was monitored by TLC (using EA:Hexane 1:9 as mobile phase) which confirmed that the reaction got completed after 16 h of stirring at room temperature. The resulting reaction mixture was quenched using NH₄Cl extracted with ethyl acetate (2×30 mL). The combined organic layers dried over Na₂SO₄ and concentrated under reduced pressure to afford 25 g crude. The crude was purified by manual column chromatography using silica (60-120 mesh) as stationary phase (gradient elution 4% EA in hexane) to afford 8-methylene-1,4-dioxaspiro[4.5]decane (A36) (18 g, 116.88 mmol, Yield 72.92%)

¹H NMR (CDCl₃, 400 MHz): δ ppm 4.69 (s, 2H), 3.98 (s, 4H), 2.30 (t, J=6.4 Hz, 4H), 1.72 (t, J=6.8 Hz, 4H).

### Step-2

To a stirred solution of A36 (5.2 g, 34 mmol, 1.0 eq), Diethylzinc (87.9 mL, 87.9 mmol, 2.56 eq) in toluene (52 mL) was added to the reaction mixture under N₂ atmosphere at −40° C. Then the resulting mixture was stirred at −40° C. for 20 min. The CH₂I₂ (46.06 g, 172 mmol, 5.2 eq), was added at −40° C. and reaction mixture was allowed to run overnight. The reaction was monitored by TLC (EA:Hexane 1:9 as mobile phase) which confirmed that the reaction got completed after 18 h of stirring at room temperature. The resulting reaction mixture was quenched using NH₄Cl extracted with diethyl ether (4×30 mL). The combined organic layers were washed with sodium thiosulfate (3×40 ml) and dried over Na₂SO₄ then concentrated under reduced pressure to afford 5.3 g crude. The crude was purified by manual column chromatography using silica (230-400 mesh) as stationary phase (gradient elution 3% Ethyl acetate in hexane) yielding 7,10-dioxadispiro[2.2.4.2]dodecane (A37) (3 g, 17.85 mmol, Yield 52%)

¹H NMR (CDCl₃, 400 MHz): δ ppm 3.98 (s, 4H), 1.71 (t, J=6.0 Hz, 4H), 1.45-1.42 (m, 4H), 0.30 (s, 4H).

### Step 3

To a stirred solution of A37 (1.1 g, 6.54 mmol, 1.0 eq) in THF:H₂O (8.33 mL, 3:2) was added TFA (1.67 mL, 21.58 mmol, 3.3 eq) to the reaction mixture under N₂ atmosphere at 0° C. The resulting mixture was stirred at 0° C. for 18 h. The reaction was monitored by TLC (using Diethyl ether:pentane 1:9 as mobile phase) which confirmed that the reaction got completed after 18 h of stirring at room temperature. After completion the reaction mixture was quenched using aq. Na₂CO₃ extracted with diethyl ether (4×30 mL). The combined organic layers were washed with Na₂SO₃ (3×40 ml) and dried over Na₂SO₄, concentrated under reduced pressure to afford 5.3 g crude. The crude was purified by manual column chromatography using silica (230-400 mesh) as stationary phase (2% Ethyl acetate in hexane) yielding spiro[2.5]octan-6-one (A38) (0.2 g, 1.61 mmol, Yield 24.63%)

¹H NMR (CDCl₃, 400 MHz): δ ppm 2.54-2.42 (m, 4H), 1.70 (t, J=6.8 Hz, 4H), 0.51 (s, 4H).

### Step 4

To a stirred solution of A38 (0.2 g, 1.61 mmol, 1.0 eq) in THF (4 mL) was added LiHMDS (1.7 mL, 1.77 mmol, 1.1 eq, 1.0 M in THF) dropwise under N₂ atmosphere at −78° C. Then the resulting mixture was stirred at −78° C. for 1 h. After 1 h. N-Phenyl-bis(trifluoromethanesulfonimide) CAS: 37595-74-7 (0.68 g, 1.93 mmol, 1.2 eq) was added to the reaction mixture and the reaction was allowed to run overnight. The reaction was monitored by TLC (ethyl acetate:hexane 1:9 as mobile phase) which confirmed that the reaction got completed after 18 h of stirring at room temperature. The resulting reaction mixture was quenched using H₂O and extracted with ethyl acetate (4×30 mL). The combined organic layers were dried over Na₂SO₄ and concentrated under reduced pressure to afford 0.25 g crude. The crude was purified by manual column chromatography using silica (230-400 mesh) as stationary phase (gradient elution 5% Ethyl acetate in hexane) to afford spiro[2.5]oct-5-en-6-yl trifluoromethanesulfonate (A39) (0.19 g, 0.74 mmol, Yield 46.04%)

¹H NMR (CDCl₃, 400 MHz): δ ppm 8.81-5.79 (m, 1H), 2.45-2.40 (m, 2H), 2.07-2.04 (m, 2H), 1.57 (t, J=6.4 Hz, 2H), 0.41 (s, 4H).

### Step 5

To a stirred solution of A39 (1.4 g, 5.46 mmol, 1.0 eq) in Dioxane (14 MI) was added B₂Pin₂ (CAS: 73183-34-3) (1.52 g, 6.0 mmol, 1.1 eq) followed by addition of KOAc (1.62 g, 16.54 mmol, 3.03 eq), PdCl₂ (dppf) (0.39 g, 0.54 mmol, 0.1 eq) and reaction mixture was purged with N₂ for 10 min then stirred overnight at 100° C. TLC (ethyl acetate:hexane 1:9 as mobile phase) confirmed the completion of reaction. The resulting reaction mixture was filtered using celite and extracted with ethyl acetate (4×30 Ml). The combined organic layers were dried over Na₂SO₄ and concentrated under reduced pressure to afford 0.41 g crude. The crude was purified by manual column chromatography using silica (230-400 mesh) as stationary phase (10% Ethyl acetate in hexane) yielding 4,4,5,5-tetramethyl-2-(spiro[2.5]oct-5-en-6-yl)-1,3,2-dioxaborolane (A40) (0.6 g, 2.56 mmol, Yield 46.90%)

¹H NMR (CDCl₃, 400 MHz): δ ppm 6.61-6.60 (m, 1H), 2.25-2.21 (m, 2H), 2.01-1.99 (m, 2H), 1.39-1.36 (m, 2H), 1.29 (s, 1H), 0.32-0.28 (m, 4H).

### Step 6

To a stirred solution of A40 (0.07 g, 0.29 mmol, 1.1 eq) and A23 (0.08 g, 0.27 mmol, 1.0 eq) in Dioxane:H₂O (8:2) was added K₃PO₄ (0.173 g, 0.81 mmol, 3.0 eq), PdCl₂ (dppf)·DCM (0.11 g, 0.31 mmol, 0.5 eq) and the reaction mixture was allowed to run overnight at 110° C. The reaction was monitored by TLC (ethyl acetate:hexane 5:5 as mobile phase) which confirmed that the reaction got completed after 16 h of stirring at 110° C. The resulting reaction mixture was filtered using celite and the filtrate was extracted with ethyl acetate (4×30 mL). The combined organic layers were dried over Na₂SO₄ and concentrated under reduced pressure to afford 0.078 g crude. The crude was purified by manual column chromatography using silica (230-400 mesh) as stationary phase (gradient elution 35% Ethyl acetate in hexane) yielding N-isopropyl-8-(spiro[2.5]oct-5-en-6-yl)quinoline-3-carboxamide (Compound 59) (0.03 g, 0.09 mmol, Yield 31.32%) 1H NMR (MeOD, 400 MHz): δ ppm 9.23 (d, J=2.4 Hz, 1H), 8.72 (d, J=2.4 Hz, 1H), 7.93 (dd, J=8.0, 1.6 Hz, 1H), 7.68-7.61 (m, 2H), 5.90-5.88 (m, 1H), 4.33-4.26 (m, 1H), 2.69-2.65 (m, 2H), 2.20-2.18 (m, 2H), 1.68-1.65 (m, 2H), 1.33-1.30 (m, 6H), 0.44 (s, 4H). Note: —CONH proton exchanged in MeOD NMR.

LCMS (Method A)-2.57 min, 100%, 254.0 nm MS; ES+321.2 (M+H)

HPLC (Method A)-9.81 min, 95.41%, 210 nm

### Example 56—Synthesis of(S)—N-(1-(pyridin-2-yl)ethyl)-8-(spiro[2.5]oct-5-en-6-yl)quinoline-3-carboxamide (Compound 60)

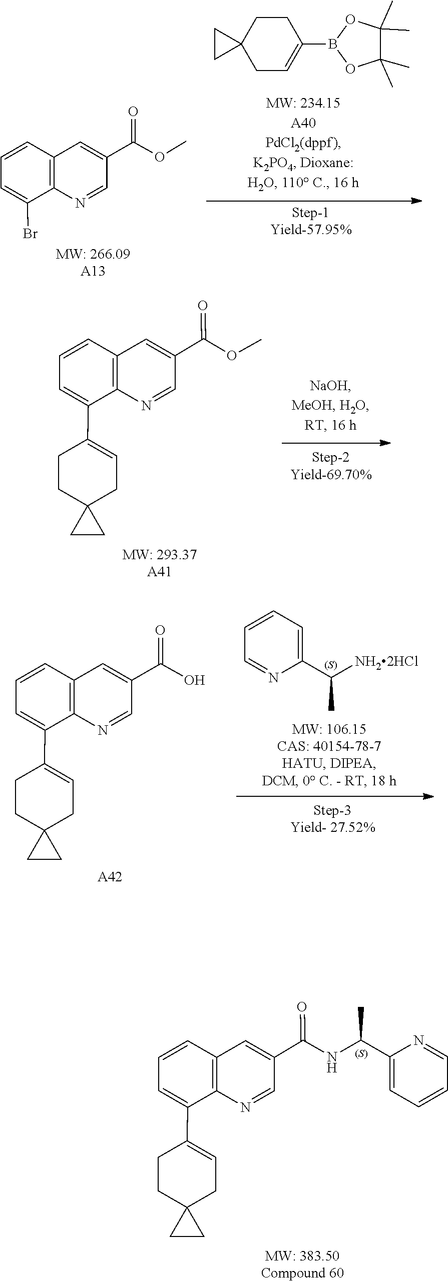

### Step-1

To a stirred solution of methyl 8-bromoquinoline-3-carboxylate (A13) (1.8 g, 6.76 mmol, 1.0 eq), A40 (1.74 g, 7.44 mmol, 1.1 eq) in Dioxane:H₂O (8:2) was added K₃PO₄ (4.28 g, 20.2 mmol, 3.0 eq) and purged with N₂ for 15 min. Then PdCl₂(dppf) (0.24 g, 0.338 mmol, 0.05 eq), was added to the reaction mixture then stirred at 110° C. for 16 h. TLC (EA:Hexane 1:1 as mobile phase) indicated completion of reaction. The resulting reaction mixture was filtered using celite then filtrate was extracted with ethyl acetate (2×30 mL). The combined organic layers were dried over Na₂SO₄ and concentrated under reduced pressure to afford 1.75 g crude. The crude was purified by manual column chromatography using silica (60-120 mesh) as stationary phase (gradient elution 30% Ethyl acetate in hexane) to afford methyl 8-(spiro[2.5]oct-5-en-6-yl)quinoline-3-carboxylate (A41) (1.15 g, 3.91 mmol, Yield 57.95%)

¹H NMR (MeOD, 400 MHz): δ ppm 9.36 (d, J=2.0 Hz, 1H), 8.96 (d, J=2.0 Hz, 1H), 7.98 (dd, J=8.0 Hz, 1.6 Hz, 1H), 7.72 (dd, J=6.8 Hz, 1.2 Hz, 1H), 7.65 (t, J=8.0 Hz, 1H), 5.90-5.88 (m, 1H), 4.03 (s, 3H), 2.68-2.67 (m, 2H), 2.20-2.18 (m, 2H), 1.67 (t, J=6.0 Hz, 2H), 0.45 (s, 4H).

LCMS (Method A): 2.982 min, 99.11%, 254.0 nm, MS: ES+294.2 (M+1)

### Step 2

To a stirred solution of A41 (1.1 g, 3.74 mmol, 1.0 eq) in MeOH:H₂O (8:1) (11 ml) was added NaOH (0.74 g, 18.74 mmol, 5.0 eq) at RT. Then the resulting mixture was stirred at room temperature for 16 h. The reaction was monitored by TLC (using 100% ethyl acetate as mobile phase) which confirmed that the reaction got completed after 16 h of stirring at room temperature. The resulting reaction mixture was concentrated under vacuum then residue was acidified (PH=3) with dil. HCl. The solid obtained was filtered and dried under vacuum to afford 8-(spiro[2.5]oct-5-en-6-yl)quinoline-3-carboxylic acid (A42) (0.73 g, 2.61 mmol, Yield 69.70%)

¹H NMR (DMSO, 400 MHz): δ ppm 9.36 (d, J=2.0 Hz, 1H), 8.94 (d, J=2.0 Hz, 1H), 7.97 (dd, J=8.0 Hz, 1.2 Hz, 1H), 7.70 (dd, J=6.8 Hz, 1.2 Hz, 1H), 7.63 (t, J=7.6 Hz, 1H), 5.90-5.88 (m, 1H), 2.68-2.65 (m, 2H), 2.20-2.18 (m, 2H), 1.66 (t, J=6.0 Hz, 2H), 0.44 (s, 4H). Note: —COOH proton might be exchanged in MeOD NMR.

LCMS (Method A): 2.510 min, 99.38%, 254.0 nm, MS: ES+280.11 (M+1)

### Step 3

To a stirred solution of A42 (0.09 g, 0.322 mmol, 1.0 eq) in DCM (1 mL) was added HATU (0.18 g, 0.48 mmol, 1.5 eq) followed by addition of DIPEA (0.12 g, 0.96 mmol, 3.0 eq) under N₂ atmosphere. Then after 10 min CAS: 40154-78-7 (S)-1-(pyridin-2-yl)ethanamine dihydrochloride (0.037 g, 0.35 mmol, 1.1 eq) was added and the resulting mixture was stirred at RT for 18 h. The reaction was monitored by TLC (100% EA mobile phase) which indicated completion of reaction. The resulting reaction mixture was poured into water (10 mL) and extracted with ethyl acetate (2×30 mL). The combined organic layers were dried over Na₂SO₄ and concentrated under reduced pressure to afford the crude product. The crude was purified by manual column chromatography using silica (100-200 mesh) as stationary phase (gradient elution 100% Ethyl acetate) to afford (S)—N-(1-(pyridin-2-yl)ethyl)-8-(spiro[2.5]oct-5-en-6-yl)quinoline-3-carboxamide (Compound 60) (0.034 g, 0.088 mmol, Yield 27.52%)

¹H NMR (MeOD, 400 MHz): δ ppm 9.28 (d, J=2.4 Hz, 1H), 8.83 (d, J=2.4 Hz, 1H), 8.55 (d, J=4.0 Hz, 1H), 7.95 (d, J=8.0 Hz, 2.0 Hz, 1H), 7.85 (dt, J=7.6 Hz, 1.6 Hz, 1H), 7.69-7.64 (m, 2H), 7.54 (d, J=7.6 Hz, 1H), 7.35-7.32 (m, 1H), 5.90-5.88 (m, 1H), 5.35 (q, J=7.2 Hz, 1H), 2.69-2.65 (m, 2H), 2.20-2.18 (m, 2H), 1.68-1.65 (m, 5H), 0.44 (s, 4H). Note: —CONH proton exchanged in MeOD NMR.

LCMS (Method A): 2.273 min, 100%, 254.0 nm, MS: ES+384.2 (M+1)

HPLC (Method A): 9.67 min, 99.33%, 254.0 nm.

Chiral HPLC (Method A): 7.82 min, 94.27%, 300 nm.

### Example 57—Synthesis of N-(oxazol-2-ylmethyl)-8-(spiro[2.5]oct-5-en-6-yl)quinoline-3-carboxamide (Compound 61)

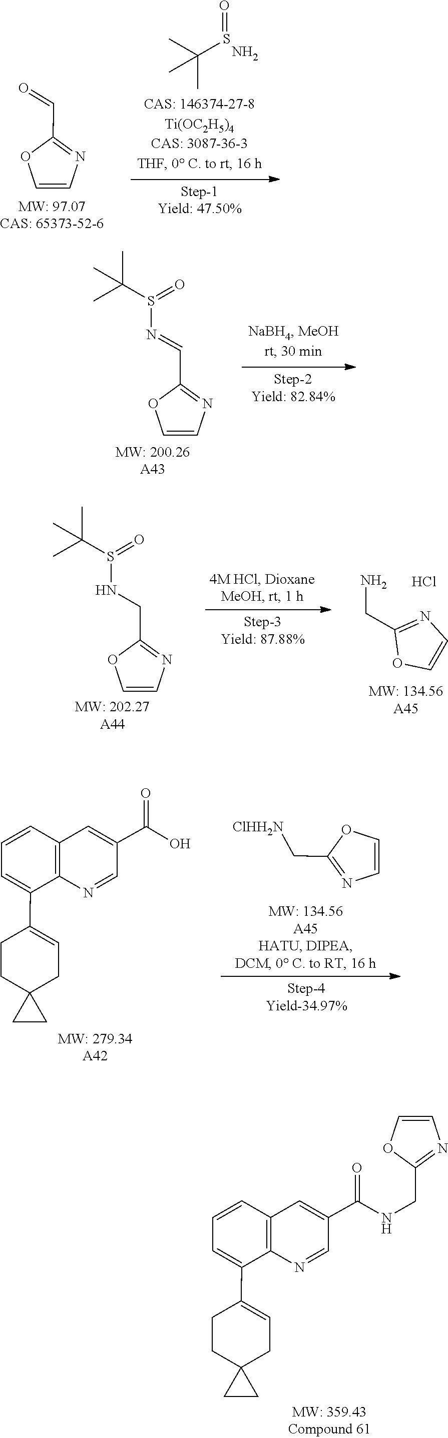

### Step-1

To a stirred solution of oxazole-2-carbaldehyde (5.0 g, 51.5 mmol, 1.0 eq) in DMF (10 mL) was added 2-Methylpropane-2-sulfinamide CAS: 146374-27-8 (7.4 g, 61.8 mmol, 1.2 eq), and Piperidine-1-carbaldehyde CAS: 3087-36-3 (25.5 ml, 10.3 mmol, 2.0 eq) at 0° C. under Nitrogen atmosphere. Then reaction mixture was stirred at RT for 16 h. The TLC (50% EtOAc in Hexane as a mobile phase) indicated the completion of reaction. The resulting reaction mixture was poured onto water (100 mL) and extracted with EtOAc (3×200 mL). The combined organic layer was dried over Na₂SO₄, filtered and concentrated under reduced pressure to obtained the crude. The resulting crude product was purified by column chromatography using silica gel 230-400 mesh size and desired product eluted in 40% EtOAc in hexane to afford (E)-2-methyl-N-(oxazol-2-ylmethylene) propane-2-sulfinamide (A43) (4.9 g, 24.46 mmol, Yield: 47.50%).

¹H NMR (DMSO, 400 MHz): δ ppm 8.45 (s, 1H), 8.28 (s, 1H), 7.62 (s, 1H), 1.27 (s, 9H).

LCMS (Method A): LCMS does not support the desired product mass.

### Step-2

To a stirred solution of (E)-2-methyl-N-(oxazol-2-ylmethylene) propane-2-sulfinamide (A43) (4.9 g, 29.9 mmol, 1.0 eq) in MeOH (20 mL) was added NaBH₄ (1.7 g, 44.9 mmol, 2.0 eq) at 0° C. The reaction mixture was stirred at RT for 30 min and monitored on TLC (5% DCM:MeOH as mobile phase). The resulting reaction mixture was poured into water (400 mL) and extracted with EtOAc (3×200 mL). The combined organic layer was dried over Na₂SO₄, filtered and concentrated under reduced pressure to obtained the crude. The resulting crude product was purified by column chromatography (desired product eluted in 2% MeOH in DCM) to afford 2-methyl-N-(oxazol-2-ylmethyl) propane-2-sulfinamide (A44) (4.1 g, 20.26 mmol, Yield: 82.84%).

LCMS (Method A): 1.303 min, 100.0%, 210.0 nm MS: ES+203.0 (M+1).

¹H NMR (DMSO, 400 MHz): δ ppm 7.64 (s, 1H), 7.09 (s, 1H), 4.49-4.37 (m, 2H), 3.94 (t, J=8.8 Hz 1H), 1.24 (s, 9H).

### Step-3

To a stirred solution of 2-methyl-N-(oxazol-2-ylmethyl) propane-2-sulfinamide (A44) (9.0 g, 44.4 mmol, 1.0 eq) in MeOH (90 mL) was added 4M HCl in Dioxane (11.68 g, 31.1 mmol, 7.0 v) at 0° C. and reaction mixture was stirred at RT for 1 h. The reaction was monitored on TLC (using; 5% DCM:MeOH as mobile phase). After completion, the resulting reaction mixture was directly concentrated under reduced pressure to obtain the crude material which was triturated using pentane to afford oxazol-2-ylmethanamine hydrochloride (A45) (7.0 g, 52.02 mmol, Yield: 87.88%).

LCMS (Method A): LCMS does not support the desired product mass

¹H NMR (DMSO, 400 MHz): δ ppm 8.94 (s, 3H), 8.23 (s, 1H), 7.30 (s, 1H), 4.22 (s, 2H).

### Step-4

To a stirred solution of A42 (0.2 g, 0.71 mmol, 1.0 eq) in DCM (2 mL) was added HATU (0.40 g, 1.07 mmol, 1.5 eq) and DIPEA (0.27 g, 2.1 mmol, 3.0 eq) at 0° C. then after 10 min (A45) (0.12 g, 0.93 mmol, 1.3 eq) was added to the reaction mixture under N₂ atmosphere. The resulting reaction mixture was stirred at room temperature for 16 h. The TLC (100% EA as mobile phase) indicates completion of reaction. The resulting reaction mixture was poured onto ice water (10 mL) and extracted with ethyl acetate (2×20 mL). The combined organic layers were dried over Na₂SO₄ and concentrated under reduced pressure to afford 0.21 g crude. The crude was purified by manual column chromatography using silica (100-200 mesh) as stationary phase (gradient elution 100% Ethyl acetate) yielding N-(oxazol-2-ylmethyl)-8-(spiro[2.5]oct-5-en-6-yl)quinoline-3-carboxamide (Compound 61) (0.09 g, 0.25 mmol, Yield 34.97%)

¹H NMR (MeOD, 400 MHz): δ ppm 9.29 (d, J=2.4 Hz, 1H), 8.80 (d, J=2 Hz, 1H), 7.96-7.93 (m, 2H), 7.70-7.62 (m, 2H), 7.18 (s, 1H), 5.89 (br s, 1H), 4.80 (s, 2H), 2.68-2.67 (m, 2H), 2.19 (d, J=3.2 Hz, 2H), 1.67 (t, J=6.0 Hz, 2H), 0.44 (s, 4H). Note: —CONH proton exchanged in MeOD NMR.

¹H NMR (DMSO, 400 MHz): δ ppm 9.51 (t, J=5.6 Hz, 1H), 9.28 (d, J=2.0 Hz, 1H), 8.84 (d, J=2.0 Hz, 1H), 8.09 (s, 1H), 8.00-7.98 (m, 1H), 7.67-7.61 (m, 2H), 7.19 (s, 1H), 5.89 (br s, 1H), 4.67 (d, J=5.6 Hz, 2H), 2.69 (br s, 2H), 2.13 (br s, 2H), 1.55 (t, J=6.0 Hz, 2H), 0.40 (s, 4H).

LCMS (Method A)-2.38 min, 100%, 254.0 nm, MS: ES+360.3 (M+1)

HPLC (Method A)-8.76 min, 97.68%, 210 nm

### Example 58—N—((S)-1-hydroxy-3-methoxypropan-2-yl)-8-(4-(trifluoromethyl)cyclohex-1-en-1-yl)quinoline-3-carboxamide (Compound 62)

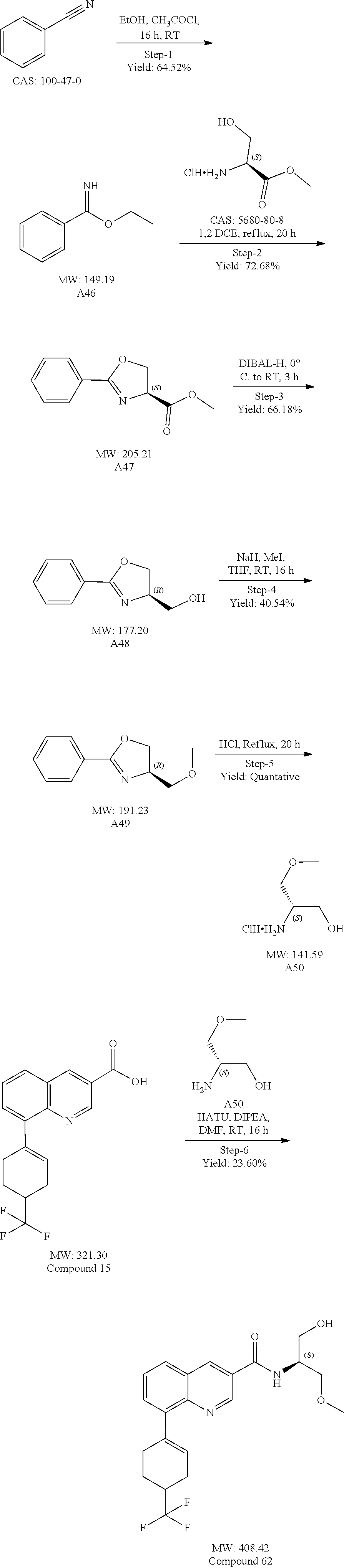

### Step-1

A solution of benzonitrile CAS 100-47-0 (15 g, 145.63 mmol, 1.0 eq) and acetyl chloride (83 ml, 1165.04 mmol, 8.0 eq), in EtOH (75 mL) was stirred at RT for 16 h. The reaction was monitored on TLC (using EtOAc:Hex; 3:7 as mobile phase) which confirmed that the reaction got completed after 16 h. The resulting reaction mixture was quenched with Sat. NaHCO₃ (30 mL) and extracted with EtOAc (3×50 mL). The combined organic layers were dried over Na₂SO₄ and concentrated under reduced pressure to afford 20.0 g crude. The obtained crude material was purified by manual column chromatography using silica (100-200 mesh) as stationary phase (gradient elution 10% EA in Hex) to afford ethyl benzimidate (A46) (14 g, 93.95 mmol, Yield: 64.52%).

¹H NMR (DMSO-d6, 400 MHz): δ ppm, 8.89 (s, 1H), 7.83-7.81 (m, 2H), 7.51-7.42 (m, 3H), 4.24 (q, J=7.2 Hz, 2H), 1.31 (t, J=7.2 Hz, 3H)

LCMS (Method A): 1.081 min, 91%, 254.0 nm, MS: ES+149 (M+1)

### Step-2

A solution of ethyl benzimidate (A46) (14 g, 93.95 mmol, 1.0 eq) and methyl L-serinate hydrochloride CAS 5680-80-8 (16 g, 103.35 mmol, 1.1 eq) in 1,2 DCE (140 mL) was stirred at 80° C. for 20 h. TLC (EA:Hex; 1:1 as mobile phase) indicated completion of reaction then resulting reaction mixture was filtered through celite bed and filtrate was concentrate under reduce pressure to obtained the residue. The obtained crude residue was purified by manual column chromatography using silica (100-200 mesh) as stationary phase (gradient elution 30% EA in Hex) to afford(S)-2-phenyl-4,5-dihydrooxazole-4-carboxylate (A47) (14 g, 68.292 mmol, Yield: 72%).

¹H NMR (DMSO-d6, 400 MHz): δ ppm, 7.9-7.86 (m, 2H), 7.61-7.57 (m, 1H), 7.52-7.48 (m, 2H), 4.99-4.95 (dd, J=10 Hz, 8 Hz, 1H), 4.64-4.56 (m, 2H), 3.71 (s, 3H),

LCMS (Method A): 1.799 min, 99.47%, 254.0 nm, MS: ES+206 (M+1)

### Step-3

To a stirred solution of(S)-2-phenyl-4,5-dihydrooxazole-4-carboxylate (A47) (14 g, 68.292 mmol, 1.0 eq) in THF (1.8 mL) was added DIBAL-H (204 ml, 204.87 mmol, 3.0 eq) at 0° C., The resulting reaction mixture was stirred for 3 h at room temperature. The reaction was monitored on TLC (EA:Hex; 7:2 as mobile phase) which confirmed that the reaction got completed after 3 h. The resulting reaction mixture was quenched with Sat. NH₄Cl (50 mL) and extracted with EtOAc (3×100 mL). The combined organic layers were dried over Na₂SO₄ and concentrated under reduced pressure to afford 15 g crude. The obtained crude material was purified by Manual column using silica (100-200 mesh) as stationary phase (70% EA in Hex) to afford (R)-(2-phenyl-4,5-dihydrooxazol-4-yl) methanol (A48) (8.0 g, 45.197 mmol, Yield: 66%).

¹H NMR (DMSO-d6, 400 MHz): δ ppm, 7.86 (d, J=7.2 Hz, 2H), 7.56-7.45 (m, 3H), 4.9 (t, J=5.6 Hz, 1H), 4.32-4.24 (s, 3H), 3.62-3.44 (m, 2H).

LCMS (Method A): 1.006 min, 94%, 254.0 nm, MS: ES+178 (M+1)

### Step-4

To a stirred solution of (R)-(2-phenyl-4,5-dihydrooxazol-4-yl) methanol (A48) (8.0 g, 45.197 mmol, 1.0 eq) in THF (1 ml) was added NaH (3.6 g, 90.39 mmol, 2.0 eq) and Mel (16.04 g, 112.9 mmol, 2.5 eq) at 0° C. then reaction mixture was stirred at RT for 16 h. The reaction was monitored by TLC (EA:HEX; 1:1 as mobile phase) which indicated the completion of reaction. The resulting reaction mixture was poured onto ice cold water (50 mL) and extracted with EtOAc (3×100 mL) dried over Na₂SO₄ and concentrated under reduced pressure to afford the crude. The obtained crude material was purified by manual column using silica (100-200 mesh) as stationary phase (gradient elution 50% EA in Hex) yielding (R)-4-(methoxymethyl)-2-phenyl-4,5-dihydrooxazole (A49) (3.5 g, 18.324 mmol, Yield: 40%).

¹H NMR (DMSO-d⁶, 400 MHz): δ ppm, 7.87-7.85 (m, 2H), 7.57-7.46 (m, 3H), 4.49-4.39 (m, 2H), 4.21 (t, J=7.2 Hz, 1H), 3.52 (dd, J=9.6, 4 Hz, 1H), 3.42 (dd, J=9.6, 4 Hz, 1H), 3.29 (s, 3H).

LCMS (Method A): 1.345 min, 88%, 254.0 nm, MS: ES+192 (M+1)

### Step-5

A solution of (R)-4-(methoxymethyl)-2-phenyl-4,5-dihydrooxazole (A49) (14.0 g, 73.210 mmol, 1.0 eq) in 4.0 M aq. HCl (420 mL) was stirred at RT for 5 min then resulting reaction mixture was heated at 110° C. for 20 h. TLC (5% MeOH in DCM as mobile phase) indicated the completion of reaction. The resulting reaction mixture was cooled to RT then filtered through Buchner funnel. Obtained filtrate was extracted with ether (4×100 mL) to remove the impurities then combined aqueous layer was concentrated under reduced pressure to afford 12.0 g (S)-2-amino-3-methoxypropan-1-ol hydrochloride (A50) (12.0 g, 114.12 mmol, Yield: Quantative).

¹H NMR (DMSO-d⁶, 400 MHz): δ ppm 8.31 (br s, 3H), 3.57-3.47 (m, 5H), 3.27 (s, 3H), 3.21-3.20 (m, 1H),

LCMS (Method A): 0.197 min, 70.29%, 210.0 nm, MS: ES+106.0 (M+1)

### Step-6

To a solution of 8-(4-(trifluoromethyl)cyclohex-1-en-1-yl)quinoline-3-carboxylic acid (Compound 15) (0.5 g, 1.557 mmol, 1.0 eq.), HATU (0.887 g, 2.334 mmol, 1.5 eq.) and DIPEA (0.8 mL, 4.669 mmol, 3.0 eq.) in DMF (5 mL) at 0° C. under nitrogen atmosphere stirred for 20 min, was added (S)-2-amino-3-methoxypropan-1-ol (A50) (0.327 g, 3.115 mmol, 2.0 eq.) under nitrogen atmosphere. The resulting mixture was stirred for 16 h at room temperature. TLC indicated the completion of reaction; the resulting reaction mixture was quenched with ice-cold water (10 mL) and extracted with EtOAc (3×30 mL). The combined organic layers were dried over Na₂SO₄ and concentrated under reduced pressure to afford crude. The obtained crude material was purified by flash column chromatography using silica (230-400 Mesh) as stationary phase (70% Ethyl acetate in hexane) to afford N—((S)-1-hydroxy-3-methoxypropan-2-yl)-8-(4-(trifluoromethyl)cyclohex-1-en-1-yl)quinoline-3-carboxamide (Compound 62) (0.15 g, 0.367 mmol, Yield: 23.60%).

¹H NMR (DMSO-d₆, 400 MHz): δ ppm, 9.25 (m, 1H), 8.28 (m, 1H), 8.53 (d, J=8.0 Hz, 1H), 8.01-7.99 (m, 1H), 7.64-7.63 (m, 2H), 5.87 (br s, 1H), 4.83 (t, J=5.6 Hz, 1H), 4.21-4.18 (m, 1H), 3.55-3.50 (m, 1H), 3.31 (s, 3H), 2.72-2.68 (m, 3H), 2.32-2.07 (m, 3H), 1.71-1.67 (m, 1H). Note: CF3 CH proton merged in DMSO Solvent peak.

¹H NMR (MeOD, 400 MHz): δ ppm, 9.25 (m, 1H), 8.79 (m, 1H), 7.97 (dd, J=7.6 Hz, J=8.0 Hz, 1H), 7.69-7.63 (m, 2H), 5.87 (br s, 1H), 4.39 (t, J=5.6 Hz, 1H), 3.80-3.76 (m, 2H), 3.67-3.64 (m, 2H), 3.63 (s, 3H), 2.78-2.15 (m, 6H), 1.91-1.85 (m, 1H). Note: —CONH and —OH proton exchanged in MeOD NMR.

LCMS (Method A): 2.215 min, 100%, 254.0 nm, MS: ES+409.42 (M+1)

HPLC (Method A): 7.84 min, 100%, 254.0 nm

### Example 59—Synthesis of N—(R)-4aminobutan-2-yl)-8-(4-(trifluoromethyl)cyclohex-1-en-1-yl)quinoline-3-carboxamide (Compound 63)

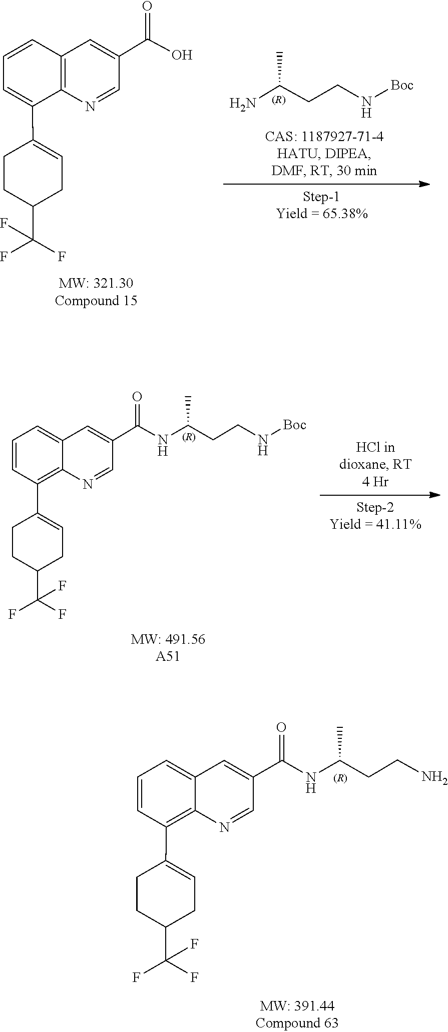

### Step-1:

To a solution of 8-(4-(trifluoromethyl)cyclohex-1-en-1-yl)quinoline-3-carboxylic acid (Compound 15) (0.2 g, 0.62 mmol, 1.0 eq), HATU (0.35 g, 0.93 mmol, 1.5 eq) and DIPEA (0.24 g, 1.86 mmol, 3.0 eq) in DMF (2 mL) at 0° C. under nitrogen atmosphere stirred for 15 min, was added CAS: 1187927-71-4 (0.12 g, 0.68 mmol, 1.1 eq) under Nitrogen. The resulting mixture was stirred for 30 min at room temperature. The reaction was monitored on TLC (using EtOAc:Hexane; 7:3 as mobile phase) which confirmed that the reaction got completed after 30 min. The resulting reaction mixture was quenched with ice-cold water (50 mL) and extracted with EtOAc (3×15 mL). The combined organic layers were dried over Na₂SO₄ and concentrated under reduced pressure to afford crude. The obtained crude material was purified by flash column chromatography using silica (230-400 mesh) as stationary phase (40% Ethyl acetate in hexane) yielding tert-butyl ((3R)-3-(8-(4-(trifluoromethyl)cyclohex-1-en-1-yl)quinoline-3-carboxamido)butyl) carbamate (A51) (0.2 g, 0.40 mmol, Yield: 65.38%).

¹H NMR (DMSO d⁶, 400 MHz): δ ppm, 9.24 (d, J=2.0 Hz, 1H), 8.78 (d, J=2.0 Hz, 1H), 8.56 (d, J=8.4 Hz, 1H), 8.00 (t, J=4.8 Hz, 1H), 7.64-7.61 (m, 2H), 6.82-6.79 (m, 1H), 5.86 (br s, 1H), 4.13-4.06 (m, 1H), 3.04-2.95 (m, 2H), 2.80-2.67 (m, 3H), 2.31-2.24 (m, 1H), 2.09-2.06 (m, 1H), 1.74-1.61 (m, 3H), 1.36 (s, 9H), 1.19 (d, J=6.4 Hz, 4H).

LCMS (Method A): 2.781 min, 100%, 254.0 nm, MS: ES+492.2 (M+1)

### Step-2

To a solution of tert-butyl ((3R)-3-(8-(4-(trifluoromethyl)cyclohex-1-en-1-yl)quinoline-3-carboxamido)butyl) carbamate (A51) (0.19 g, 0.387 mmol, 1.0 eq) in DCM (1.9 ml) was added dropwise 4M HCl in Dioxane (1 ml) at 0° C. under nitrogen atmosphere and stirred for 4 h at room temperature. The reaction was monitored on TLC (using MeOH:DCM; 0.5:9.5 as mobile phase) which confirmed that the reaction got completed after 4 h. The resulting reaction mixture was evaporated to obtain the crude. The obtained crude material was purified by flash column chromatography using silica (230-400 mesh) as stationary phase (15% MeOH:DCM) yielding N—(R)-4 aminobutan-2-yl)-8-(4-(trifluoromethyl)cyclohex-1-en-1-yl)quinoline-3-carboxamide (Compound 63) (0.062 g, 0.15 mmol, Yield: 41.11%).

¹H NMR (MeOD, 400 MHz): δ ppm, 9.45 (m, 2H), 9.05 (d, J=7.6 Hz, 1H), 8.28 (d, J=8.0 Hz, 1H), 8.03 (d, J=6.8 Hz, 1H), 7.98-7.94 (m, 2H), 6.04 (br s, 1H), 4.37-4.34 (m, 1H), 3.15-3.06 (m, 2H), 2.75-2.64 (m, 3H), 2.63-2.60 (m, 1H), 2.48-2.45 (m, 1H), 2.26-2.23 (m, 1H), 2.04-1.92 (m, 3H), 1.44 (d, J=6.8 Hz, 3H).

¹H NMR (DMSO d₆, 400 MHz): δ ppm, 9.28 (d, J=1.6 Hz, 1H), 8.88 (s, 1H), 8.79 (d, J=8.0 Hz, 1H), 8.03 (t, J=5.6 Hz, 1H), 7.89 (s, 3H), 7.67-7.66 (m, 2H), 5.875 (br s, 1H), 4.19-4.16 (m, 1H), 2.87-2.84 (m, 2H), 2.79-2.68 (m, 3H), 2.33-2.29 (m, 1H), 2.10-2.07 (m, 1H), 1.86-1.82 (m, 2H), 1.72-1.68 (m, 1H), 1.25 (d, J=6.4 Hz, 3H).

LCMS (Method A): 1.886 min, 100%, 254.0 nm, MS: ES+392.2 (M+1)

HPLC (Method A): RT=5.51 min, 100%, 270 nm.

Chiral HPLC: Peak 1 RT=8.56 min, 39.00%, 241 nm; Peak 2=RT: 9.92 min, 60.09%, 241 nm

### Example 60—Synthesis of N—((S)-1-(dimethylamino)-3-hydroxy-1-oxopropan-2-yl)-8-(4-(trifluoromethyl)cyclohex-1-en-1-yl)quinoline-3-carboxamide (Compound 64)

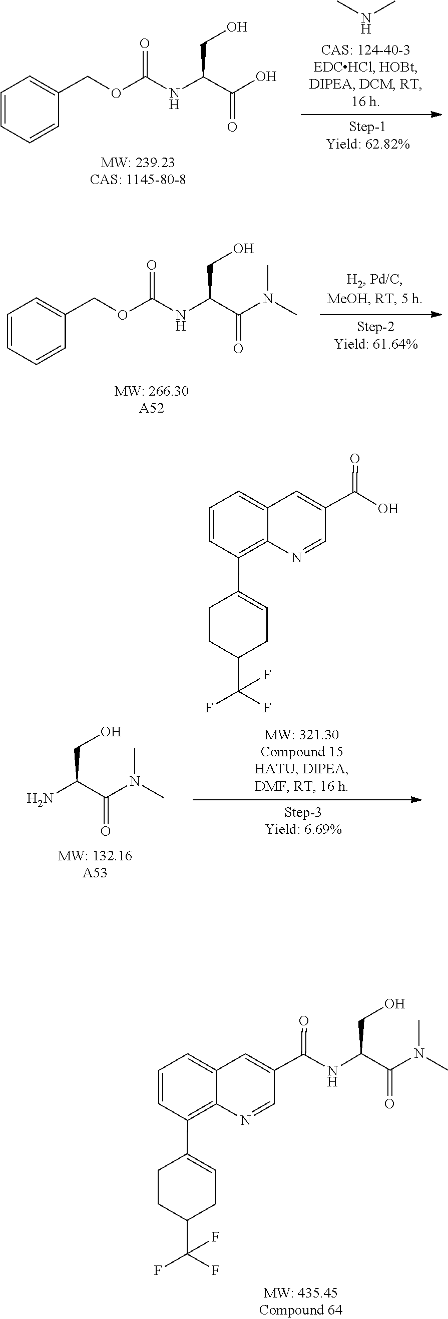

### Step-1

To a stirred solution of ((benzyloxy) carbonyl)-L-serine (CAS: 1145-80-8) (2.00 g, 8.36 mmol, 1.0 eq) in DCM (20 ml), was added 1.0 M dimethylamine CAS: 124-40-3 (0.843 ml, 12.54 mmol, 1.5 eq), followed by addition of DIPEA (4.36 ml, 25.08 mmol, 3.0 eq) and HATU (4.765 g, 12.54 mmol, 1.5 eq). The resulting mixture was stirred at room temperature for 16 h. TLC indicate the completion of reaction; the resulting reaction mixture was concentrated under reduced pressure to afford crude. The obtained crude material was purified by flash column chromatography using silica gel (230-400 mesh) as stationary phase (desired product eluted in 3% MeOH in DCM) to afford benzyl(S)-(1-(dimethylamino)-3-hydroxy-1-oxopropan-2-yl) carbamate (A52) (0.358 g, 1.34 mmol, Yield 16%)

¹H NMR (DMSO-d₆, 400 MHz): δ ppm, 7.38-7.29 (m, 6H), 5.01 (s, 2H), 4.87 (t, J=6.0 Hz, 1H), 4.54 (m, 1H), 3.57-3.53 (m, 1H), 3.46-3.42 (m, 1H), 3.05 (s, 3H), 2.82 (s, 3H).

LCMS (Method A): 1.495 min, 95.91%, 220.0 nm, MS: ES+267.15

### Step-2

To a solution of benzyl(S)-(1-(dimethylamino)-3-hydroxy-1-oxopropan-2-yl) carbamate (A52) (0.15 g, 0.56 mmol, 1.0 eq) in MeOH (5 mL) at room temperature under nitrogen atmosphere, was added Pd/C (0.075 g, 50% by wt) and stirred at room temperature under H₂ atmosphere for 5 h. TLC indicated the completion of reaction. The resulting reaction mixture was filtered through celite bed and concentrated under reduced pressure to afford the desired product A53 (S)-2-amino-3-hydroxy-N,N-dimethylpropanamide (0.078 g, 0.59 mmol, Yield: 7.44%).

¹H NMR (DMSO-d₆, 400 MHz): δ ppm, 4.77-4.68 (bs, 1H), 3.67 (t, J=7.6 Hz, 1H), 3.42-3.36 (m, 1H), 3.26-3.2 (m, 1H), 3.03 (s, 3H), 2.85 (s, 3H), 1.81-1.76 (bs, 2H),

LCMS (Method B): 0.70 min, 100%, 220.0 nm, MS: ES+133.1

### Step-3

To a stirred solution of 8-bromo-6-methoxyquinoline-3-carboxylic acid (Compound 15) (0.606 g, 1.89 mmol, 1.0 eq) in DMF (10 ml), was added(S)-2-amino-3-hydroxy-N,N-dimethylpropanamide (A53) (0.150 mg, 1.89 mmol, 1.0 eq), DIPEA (0.736 g, 5.66 mmol, 3.0 eq) and HATU (1.07 g, 2.83 mmol, 1.5 eq). The resulting mixture was stirred at room temperature for 16 h. TLC indicated the completion of reaction, the resulting reaction mixture was diluted with water (100 ml) and extracted with EtOAc (2×125 ml). Combined organic phases were dried over Na₂SO₄ and concentrated under reduced pressure to afford crude. The obtained crude material was purified by prep HPLC (Method-A) to afford N—((S)-1-(dimethylamino)-3-hydroxy-1-oxopropan-2-yl)-8-(4-(trifluoromethyl)cyclohex-1-en-1-yl)quinoline-3-carboxamide (Compound 64) (0.055 g, 0.694 mmol, 6.69%).

¹H NMR (DMSO-d₆, 400 MHz): δ ppm, 9.25 (d, J=2.0 Hz, 1H), 8.88 (m, 2H), 8.01-7.98 (m, 1H), 7.64 (m, 2H), 5.87 (br s, 1H), 5.08-5.03 (m, 1H), 4.98 (t, J=6.0 Hz, 1H), 3.79-3.73 (m, 1H), 3.67-3.62 (m, 1H), 3.15 (s, 3H), 2.97-2.87 (m, 4H), 2.87-2.80 (1H), 2.33-2.29 (m, 1H), 2.10-2.07 (m, 1H), 1.74-1.67 (m, 1H), (Note: 2 proton merged with DMSO solvent peak)

¹H NMR (MeOD, 400 MHz): δ ppm, 9.27 (d, J=2.0 Hz, 1H), 8.84 (d, J=2.0 Hz, 2H), 7.98 (dd, J=6.0, 1.6, Hz 1H), 7.70-7.63 (m, 2H), 5.87 (bs, 1H), 5.26 (t, J=4.0 Hz, 1H), 3.97-3.86 (m, 2H), 3.29 (s, 3H), 3.03 (s, 3H), 2.78-2.65 (m, 2H), 2.55-2.51 (1H), 2.40-2.22 (m, 1H), 1.92-1.89 (m, 1H), 1.88-1.86 (m, 1H).

HPLC (Method A): 7.419 min, 100%, 254.0 nm.

LCMS (Method A): 2.164 min, 100%, 254.0 nm, MS: ES+436.2

Chiral HPLC: Peak-1: 3.96, 49.9%; Peak-2: 4.90, 50% 250 nm

### Example 61—Synthesis of N-(2-hydroxy-1-(oxazol-2-yl)ethyl)-8-(4-(trifluoromethyl)cyclohex-1-en-1-yl)quinoline-3-carboxamide (Compound 65)

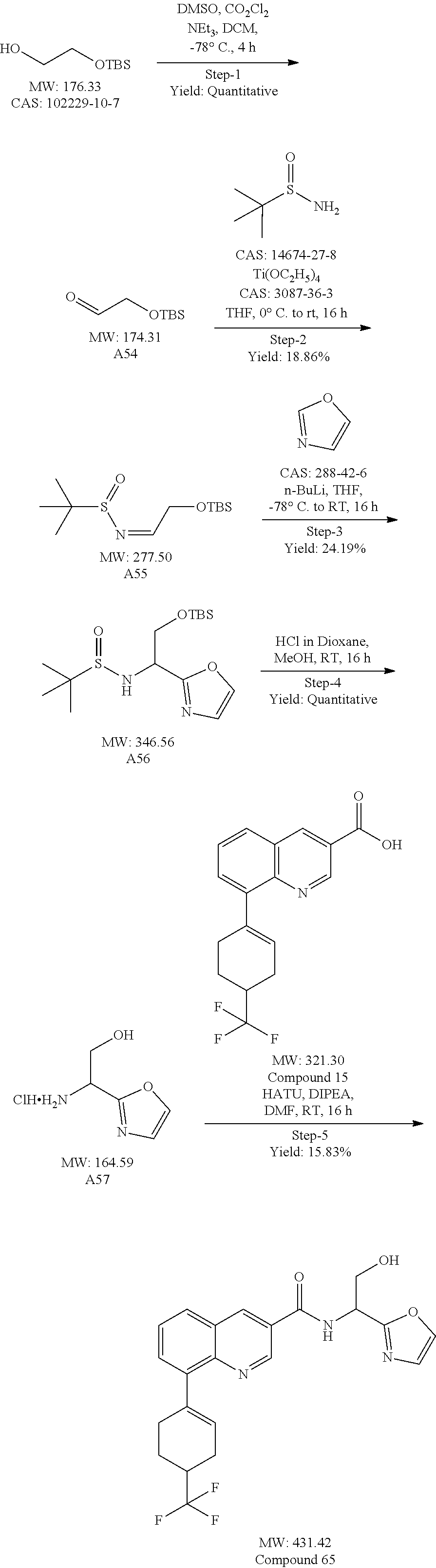

### Step-1

A stirred solution of DMSO (2.1 g, 27.21 mmol, 2.4 eq.) in DCM (40 mL) was prepared in 100 mL 3 neck RBF at −78° C. To this solution was added oxalyl chloride (1.43 g, 11.34 mmol, 1.0 eq.) at same temperature and stirred for 30 min, followed by addition of 2-((tert-butyldimethylsilyl)oxy) ethan-1-ol (CAS: 102229-10-7) (2.0 g, 11.34 mmol, 1.0 eq.) and again stirred at same temperature for another 30 min. After 30 min Et₃N (5.7 g, 56.71 mmol, 5.0 eq.) was added at −78° C. and resulting reaction mixture was stirred at room temperature for 3 h. TLC indicated completion of reaction, the resulting reaction mixture was diluted with water (100 mL) and extracted with EtOAc (3×50 mL). The combined organic layer was dried over anhy. Na₂SO₄, filtered and concentrated under reduced pressure to obtained crude 2-((tert-butyldimethylsilyl)oxy) acetaldehyde (A54) (2.2 g, 0.126 mmol, Yield: Quantitative). (The crude obtained was directly used as such for the next step without any further purification due to volatile nature of the product.)

### Step-2

A stirred solution of 2-((tert-butyldimethylsilyl)oxy) acetaldehyde (A54) (2.0 g, 11.47 mmol, 1.0 eq.), 2-methylpropane-2-sulfinamide (CAS: 146374-27-8) (1.6 g, 13.76 mmol, 1.2 eq.) in THF (50 mL) was prepared in 100 mL 3 neck RBF at 0° C. To this solution was added Ti(OC₂H₅)₄ (CAS: 3087-36-3) (5.2 g, 22.94 mmol, 2.0 eq.) at 0° C. and reaction mixture allowed to stirred at room temperature for 16 h, TLC indicated the completion of reaction, the resulting reaction mixture was diluted with water (100 mL) and extracted with EtOAc (200 mL). Then filtered through Buchner funnel and two layers were separated. The organic layer was dried over anhy. Na₂SO₄, filtered and concentrated under reduced pressure to obtain crude product. The obtained crude was purified by flash column chromatography using silica gel (100-200 mesh) (50% EtOAc in Hexanes as a mobile phase) to afford N-(2-((tert-butyldimethylsilyl)oxy)ethylidene)-2-methylpropane-2-sulfinamide (A55) (0.6 g, 2.162 mmol, Yield: 18.86%).

¹H NMR (DMSO-d₆, 400 MHz): δ ppm, 7.90 (s, 1H), 4.60 (d, J=2.8 Hz, 2H), 1.12 (s, 9H), 0.89 (s, 9H), 0.07 (s, 6H).

LCMS (Method A): 2.853 min, 99.01%, 254.0 nm, MS: ES+278.15 (M+1)

### Step-3

A stirred solution of oxazole (CAS: 288-42-6) (0.024 g, 0.360 mmol, 1.0 eq.) in THF (2 mL) was prepared in 30 mL glass vial at −78° C. To this solution was added n-BuLi (2.5 M in Hexane) (0.14 mL, 0.360 mmol, 1.0 eq.) at −78° C. and stirred for 1 h. Followed by addition of N-(2-((tert-butyldimethylsilyl)oxy)ethylidene)-2-methylpropane-2-sulfinamide (A55) (0.1 g, 0.360 mmol, 1.0 eq.) and reaction mixture was stirred at room temperature for 16 h. TLC indicated the completion of reaction, the resulting reaction mixture was quenched with aq. NH₄Cl (1 mL) and diluted with water (10 mL) and further extracted with EtOAc (3×10 mL). The combined organic layer was dried over anhy. Na₂SO₄, filtered and concentrated under reduced pressure to obtained crude product. The crude obtained product was purified by Flash Column Chromatography using silica gel (100-200 mesh) (60% EtOAc in Hexanes as a mobile phase) to afford N-(2-((tert-butyldimethylsilyl)oxy)-1-(oxazol-2-yl)ethyl)-2-methylpropane-2-sulfinamide (A56) (0.03 g, 0.086 mmol, Yield: 24.19%).

¹H NMR (DMSO-d₆, 400 MHz): δ ppm, 8.34 (s, 1H), 7.96 (s, 1H), 5.38 (d, J=8.0 Hz, 1H), 4.23-4.21 (m, 1H), 3.87-3.83 (m, 1H), 3.77-3.72 (m, 1H), 1.08 (s, 9H), 0.85 (s, 9H), 0.01 (s, 6H).

LCMS (Method A): 2.550 min, 97.60%, 220.0 nm, MS: ES+347.11 (M+1)

### Step-4

A stirred solution of N-(2-((tert-butyldimethylsilyl)oxy)-1-(oxazol-2-yl)ethyl)-2-methylpropane-2-sulfinamide (A56) (0.03 g, 0.086 mmol, 1.0 eq.) in MeOH (0.3 mL) was prepared in 5 mL single neck RBF at room temperature. To this solution was added 4M HCl in Dioxane (0.3 mL) at room temperature and resulting reaction mixture was stirred for 16 h. TLC indicated the completion of reaction, the resulting reaction mixture was directly concentrated under reduced pressure to afford 2-amino-2-(oxazol-2-yl)ethan-1-ol hydrochloride (A57) (0.02 g, Yield: Quantitative). (The crude obtained was directly used as such for the next step without any further purification).

LCMS (Method B): 0.70 min, 100.00%, 210.0 nm, MS: ES+129.1 (M+1)

### Step-5

A stirred solution of 8-(4-(trifluoromethyl)cyclohex-1-en-1-yl)quinoline-3-carboxylic acid (Compound 15) (0.175 g, 0.547 mmol, 1.0 eq.) in DMF (2 mL) was prepared in 10 mL of glass vial at room temperature. To this solution was added HATU (0.311 g, 0.820 mmol, 1.5 eq.) and DIPEA (0.212 g, 1.641 mmol, 3.0 eq.) at 0° C. and stirred reaction mixture for 10-15 min, followed by addition of A57 (0.090 g, 0.547 mmol, 1.0 eq.) at room temperature and stirred for 16 h. TLC indicated the completion of reaction, the resulting reaction mixture was diluted with water (30 mL) and EtOAc (2×30 mL). The combined organic layer was dried over anhy. Na₂SO₄, filtered and concentrated under reduced pressure to obtain the crude. The crude obtained product was purified by Flash column chromatography using silica gel (230:400 mesh) (5% MeOH in DCM as a mobile phase) to afford N-(2-hydroxy-1-(oxazol-2-yl)ethyl)-8-(4-(trifluoromethyl)cyclohex-1-en-1-yl)quinoline-3-carboxamide (Compound 65) (0.019 g, 0.044 mmol, Yield: 15.83%).

¹H NMR (DMSO-d₆, 400 MHz): δ ppm, 9.28 (d, J=1.6 Hz, 1H), 8.96 (d, J=8.4 Hz, 1H), 8.87 (d, J=2.0 Hz, 1H), 8.35 (s, 1H), 8.05 (s, 1H), 8.00 (t, J=5.6 Hz, 1H), 7.65-7.62 (m, 2H), 5.87 (br s, 1H), 5.19-5.14 (m, 1H), 5.01 (t, J=5.6 Hz, 1H), 3.84-3.73 (m, 2H), 2.81-2.67 (m, 3H), 2.29-2.25 (m, 1H), 2.09 (m, 1H), 1.74-1.64 (m, 1H). (Note: CF₃CH-proton merged with DMSO solvent peak)

¹H NMR (MeOD, 400 MHz): δ ppm, 9.28 (d, J=2.4 Hz, 1H), 8.83 (d, J=2.0 Hz, 1H), 8.22 (s, 1H), 7.98-7.96 (m, 2H), 7.70-7.63 (m, 2H), 5.86 (br s, 1H), 5.36 (t, J=6.0 Hz, 1H), 4.02-3.99 (m, 2H), 2.78-2.75 (m, 1H), 2.68-2.65 (m, 2H), 2.55-2.51 (m, 1H), 2.40-2.37 (m, 1H), 2.18-2.14 (m, 1H), 1.89-1.85 (m, 1H). Note: —NH and —OH proton exchanged with MeOD.

LCMS (Method A): 2.196 min, 97.78%, 254.0 nm, MS: ES+432.23 (M+1)

HPLC (Method A): 7.61 min, 96.07%, 254.0 nm

### Example 62—Synthesis of N-((5-oxopyrrolidin-2-yl)methyl)-8-(4-(trifluoromethyl)cyclohex-1-en-1-yl)quinoline-3-carboxamide (Compound 66)

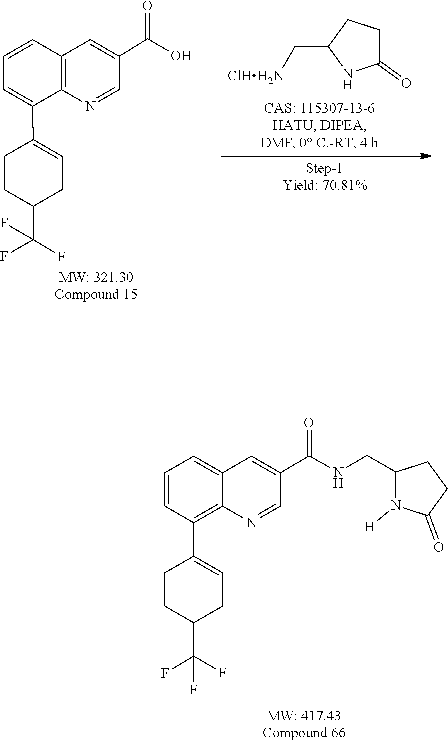

### Step-1

A stirred solution of 8-(4-(trifluoromethyl)cyclohex-1-en-1-yl)quinoline-3-carboxylic acid (Compound 15) (0.05 g, 0.15 mmol, 1.0 eq.) in DMF (0.5 mL) was prepared in 10 mL of glass vial and cooled to 0° C. To this reaction solution, HATU (0.088 g, 0.23 mmol, 1.5 eq.) was added at 0° C. under nitrogen atmosphere. After 30 min of stirring, DIPEA (0.06 g, 0.46 mmol, 3.0 eq.) and 5-(aminomethyl) pyrrolidin-2-one hydrochloride (CAS: 115307-13-6) (0.02 g, 0.15 mmol, 1.0 eq.) were added. The resulting reaction mixture was stirred at RT for 4 h. TLC indicated the completion of reaction, the resulting reaction mixture was poured on to cold water (20 mL) and precipitated out which were filtered and washed with water (50 mL) and dried under reduced pressure to obtain crude which was purified by normal phase column chromatography (desired product eluted at 7% MeOH in DCM) to afford N-((5-oxopyrrolidin-2-yl)methyl)-8-(4-(trifluoromethyl)cyclohex-1-en-1-yl)quinoline-3-carboxamide (Compound 66) (0.046 g, 0.11 mmol, Yield: 70.81%).

¹H NMR (DMSO-d₆, 400 MHz): δ ppm, 9.26 (d, J=2.0 Hz, 1H), 8.87 (t, J=5.6 Hz, 1H), 8.82 (d, J=2.4 Hz, 1H), 8.02-7.99 (m, 1H), 7.84 (s, 1H), 7.65-7.64 (m, 2H), 5.87 (br s, 1H), 3.78-3.75 (m, 1H), 3.42-3.38 (m, 2H), 2.80-2.67 (m, 2H), 2.42-2.39 (m, 2H), 2.33-2.21 (m, 2H), 2.19-2.07 (m, 2H), 1.82-1.79 (m, 1H), 1.72-1.67 (m, 1H). Note: CF₃—CH proton merged with DMSO solvent peak which is clearly observed in MeOD.

¹H NMR (MeOD, 400 MHz): δ ppm, 9.26 (d, J=2.0 Hz, 1H), 8.79 (d, J=2.0 Hz, 1H), 7.97 (dd, J=7.6 Hz, 1.6 Hz, 1H), 7.70-7.64 (m, 2H), 5.87 (br s, 1H), 4.03-4.00 (m, 1H), 3.65-3.58 (m, 2H), 3.55-3.50 (m, 1H), 2.78-2.75 (m, 1H), 2.68-2.65 (m, 2H), 2.55-2.51 (m, 1H), 2.46-2.34 (m, 3H), 2.18-2.15 (m, 1H), 2.00-1.85 (m, 2H). Note: —CONH proton exchange in MeOD Solvent.

LCMS (Method A): 2.156 min, 99.55%, 254.0 nm, MS: ES+418.2 (M+1)

HPLC (Method A): 7.39 min, 99.49%, 254.0 nm

Chiral HPLC: Peak-1: 3.57 min, 21.70%, 240.0 nm; Peak-2: 3.81 min, 27.86%, 240.0 nm; Peak-3: 5.05 min, 25.38%, 240.0 nm; Peak-4: 6.30 min, 25.0414%, 240.0 nm

### Example 63—Synthesis of N-((6-oxo-1,6-dihydropyridin-2-yl)methyl)-8-(4-(trifluoromethyl)cyclohex-1-en-1-yl)quinoline-3-carboxamide (Compound 67)

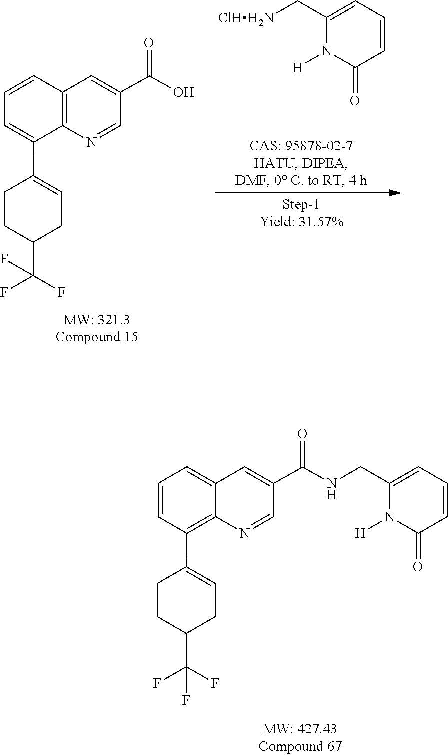

### Step-1

To a stirred solution of 8-(4-(trifluoromethyl)cyclohex-1-en-1-yl)quinoline-3-carboxylic acid (Compound 15) (0.05 g, 0.155 mmol, 1.0 eq.) in DMF (1.0 mL) was added HATU (0.088 g, 0.233 mmol, 1.5 eq.) at 0° C. and stirred for 10 minutes. Followed by addition of DIPEA (0.07 mL, 0.466 mmol, 3.0 eq.) and 6-(aminomethyl)pyridin-2 (1H)-one hydrochloride (CAS: 95878-02-7) (0.027 g, 0.171 mmol, 1.1 eq.) at 0° C. Then the resulting reaction mixture was stirred at RT for 4 h. TLC indicated the completion of reaction; the resulting reaction mixture was poured into cold water (5 mL) to obtain the precipitate. The obtained precipitate was filtered through buchner funnel and washed with water (20 mL) then dried under reduced pressure and crude was purified by column chromatography (desired product eluted in 0-2% MeOH in DCM) to afford N-((6-oxo-1,6-dihydropyridin-2-yl)methyl)-8-(4-(trifluoromethyl)cyclohex-1-en-1-yl)quinoline-3-carboxamide (Compound 67) (0.021 g, 0.049 mmol, Yield: 31.57%).

¹H NMR (DMSO-d₆, 400 MHz): δ ppm, 11.62 (br s, 1H), 9.32 (t, J=6.0 Hz, 1H), 9.29 (d, J=2.0 Hz, 1H), 8.86 (d, J=2.0 Hz, 1H), 8.04-8.01 (m, 1H), 7.66-7.63 (m, 2H), 7.39 (t, J=7.6 Hz, 1H), 6.23-6.14 (m, 2H), 5.87 (br s, 1H), 4.39 (d, J=5.2 Hz, 2H), 2.81-2.67 (m, 4H), 2.33-2.25 (m, 1H), 2.10-2.07 (m, 1H), 1.75-1.67 (m, 1H).

¹H NMR (MeOD, 400 MHz): δ ppm, 9.29 (d, J=2.0 Hz, 1H), 8.83 (d, J=2.4 Hz, 1H), 7.98 (dd, J=7.6, 1.2 Hz, 1H), 7.71-7.64 (m, 2H), 7.58 (t, J=8.8 Hz, 1H), 6.48-6.41 (m, 2H), 5.87 (br s, 1H), 4.56 (s, 2H), 2.85-2.75 (m, 1H), 2.69-2.65 (m, 2H), 2.55-2.51 (m, 1H), 2.40-2.33 (m, 1H), 2.18-2.15 (m, 1H), 1.92-1.85 (m, 1H). Note: —CONH proton exchanged in MeOD NMR.

LCMS (Method A): 2.189 min, 100.00%, 254 nm, MS: ES+428.23 (M+1)

HPLC (Method A): 7.37 min, 99.08%, 254 nm

Chiral HPLC: Peak-1 6.07 min, 49.36%, 242 nm; Peak-2 6.53 min, 49.55%, 242 nm

### Example 64—Synthesis of N-((2-oxo-1,2-dihydropyridin-3-yl)methyl)-8-(4-(trifluoromethyl)cyclohex-1-en-1-yl)quinoline-3-carboxamide (Compound 68)

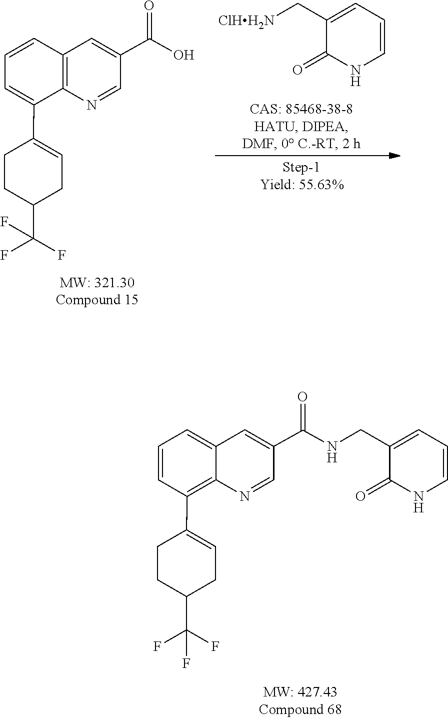

### Step-1

A stirred solution of 8-(4-(trifluoromethyl)cyclohex-1-en-1-yl)quinoline-3-carboxylic acid (Compound 15) (0.05 g, 0.15 mmol, 1.0 eq.) in DMF (0.5 mL) was prepared in 10 mL of glass vial and cooled to 0° C. To this reaction solution, HATU (0.09 g, 0.23 mmol, 1.5 eq.) was added at 0° C. under nitrogen atmosphere. After 30 min of stirring, DIPEA (0.06 g, 0.46 mmol, 3.0 eq.) and 3-(aminomethyl) 173yridine-2 (1H)-one hydrochloride (CAS: 85468-38-8) (0.02 g, 0.15 mmol, 1.0 eq.) were added. The resulting reaction mixture was stirred at RT for 3 h. TLC indicated the completion of reaction, the resulting reaction mixture was poured onto cold water (20 mL) and precipitated out which were filtered and washed with water (50 mL) and dried under reduced pressure to obtain the crude and purified by reverse phase column chromatography, desired product eluted at 50% water in acetonitrile to afford N-((2-oxo-1,2-dihydropyridin-3-yl)methyl)-8-(4-(trifluoromethyl)cyclohex-1-en-1-yl)quinoline-3-carboxamide (Compound 68) (0.037 g, 0.09 mmol, Yield: 55.63%).

¹H NMR (DMSO-d₆, 400 MHz): δ ppm, 11.69 (s, 1H), 9.29 (d, J=1.6 Hz, 1H), 9.16 (t, J=5.6 Hz, 1H), 8.87 (d, J=2.0 Hz, 1H), 8.02-8.00 (m, 1H), 7.65-7.64 (m, 2H), 7.35 (dd, J=6.4, 24.0 Hz, 2H), 6.18 (t, J=6.4, 1H), 5.87 (br s, 1H), 4.31 (d, J=5.6 Hz, 2H), 2.81-2.67 (m, 3H), 2.33-2.25 (m, 1H), 2.10-2.08 (m, 1H), 1.75-1.66 (m, 1H). Note: CF₃CH-proton merged with DMSO solvent peak.

¹H NMR (MeOD, 400 MHz): δ ppm, 9.29 (s, 1H), 8.81 (s, 1H), 7.97 (d, J=7.6 Hz, 1H), 7.69-7.63 (m, 3H), 7.41 (d, J=6.4 Hz, 1H), 6.43 (t, J=6.8 Hz, 1H), 5.87 (br s, 1H), 4.52 (s, 2H), 2.78-2.75 (m, 1H), 2.68-2.65 (m, 2H), 2.55-2.51 (m, 1H), 2.40-2.34 (m, 1H), 2.18-2.15 (m, 1H), 1.93-1.85 (m, 1H). Note: —CONH proton exchanged in MeOD NMR.

LCMS (Method A): 2.266 min, 95.77%, 254.0 nm, MS: ES+428.2 (M+1)

HPLC (Method A): 7.56 min, 100%, 254.0 nm

Chiral HPLC: Peak-1: 7.75 min, 49.18%, 240.0 nm; Peak-2: 8.57 min, 50.81%, 240.0 nm

### Example 65—Synthesis of N-(1-(hydroxymethyl)cyclopropyl)-8-(4-(trifluoromethyl)cyclohex-1-en-1-yl)quinoline-3-carboxamide (Compound 69)

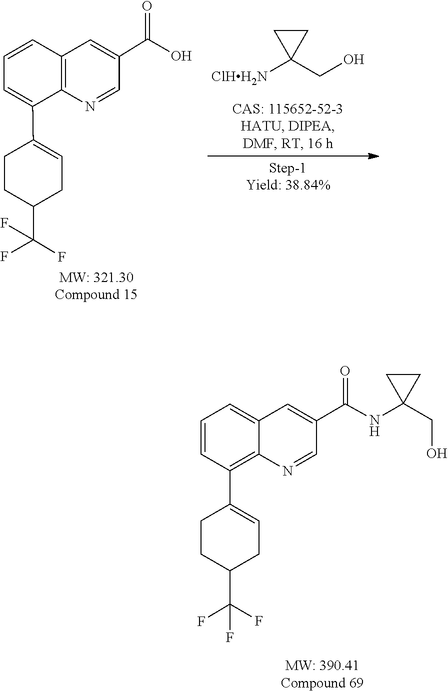

### Step-1

A stirred solution of 8-(4-(trifluoromethyl)cyclohex-1-en-1-yl)quinoline-3-carboxylic acid (Compound 15) (0.1 g, 0.31 mmol, 1.0 eq.) in DMF (1.0 mL) was prepared in 10 mL of glass vial and cooled to 0° C. To this reaction solution, was added DIPEA (0.12 g, 0.93 mmol, 3.0 eq.) stirred for 5 min then added HATU (0.17 g, 0.46 mmol, 1.5 eq.) at 0° C. under nitrogen atmosphere. After 30 min of stirring CAS: 115652-52-3 1-Amino-cyclopropanemethanol hydrochloride (0.04 g, 0.37 mmol, 1.2 eq.) was added and resulting reaction mixture was stirred at RT for 3 h. TLC indicate the completion of reaction, the resulting reaction mixture was poured onto cold water (20 mL) and extracted with ethyl acetate. Organic layer was dried over N₂SO₄ and concentrate under reduced pressure to obtain crude which was purified by normal phase flash column chromatography on silica gel (230-400 mesh) (desired product eluted at 40% Ethyl acetate in Hexane) to afford, N-(1-(hydroxymethyl)cyclopropyl)-8-(4-(trifluoromethyl)cyclohex-1-en-1-yl)quinoline-3-carboxamide (Compound 69) (0.047 g, 0.12 mmol, Yield: 38.84%)

¹H NMR (MeOD 400 MHz): δ ppm, 9.24 (d, J=2.4 Hz, 1H), 8.76 (d, J=2.0 Hz, 1H), 9.96 (dd, J=7.6, 1.6 Hz, 1H), 7.69-7.62 (m, 2H), 5.86 (br s, 1H), 3.74 (s, 2H), 2.77-2.74 (m, 1H), 2.67-2.64 (m, 2H), 2.55-2.50 (m, 1H), 2.33-2.15 (m, 1H), 2.17-2.15 (m, 1H), 1.89-1.85 (m, 1H), 0.95 (s, 4H). Note: —CONH and —OH proton exchanged in MeOD NMR.

¹H NMR (DMSO-d₆ 400 MHz): δ ppm, 9.23 (d, J=2.0 Hz, 1H), 9.01 (s, 1H), 8.80 (d, J=1.6 Hz, 1H) 7.98-7.96 (m, 1H), 7.63-7.60 (m, 2H), 5.87 (br s, 1H), 4.82 (t, J=5.6 Hz, 1H), 3.57 (d, J=5.6 Hz, 2H), 2.80-2.67 (m, 3H), 2.32-2.25 (m, 2H), 2.09-2.06 (m, 1H), 1.74-1.65 (m, 1H) 0.82-0.74 (m, 4H).

LCMS (Method A): 2.244 min, 100%, 254.0 nm, MS: ES+391.17 (M+1)

HPLC (Method A): 7.99 min, 99.74%, 254.0 nm

Chiral HPLC: Peak-1: 3.06 min, 49.18%, 240.0 nm; Peak-2: 3.50 min, 49.9%, 240.0 nm

### Example 66—Synthesis of N—((R)-1-acetamidopropan-2-yl)-8-(4-(trifluoromethyl)cyclohex-1-en-1-yl)quinoline-3-carboxamide (Compound 70)

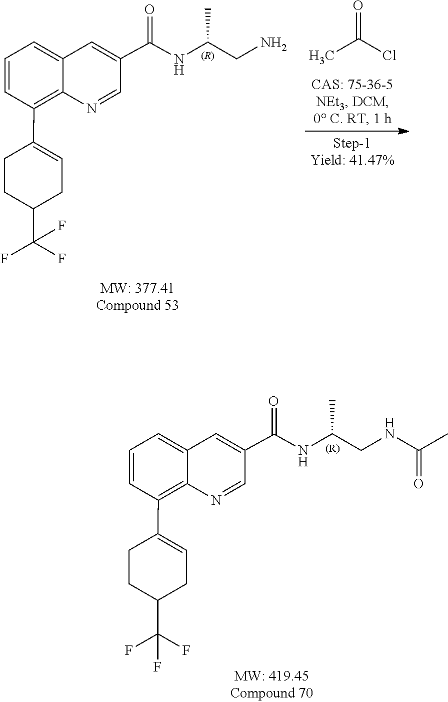

### Step-1

To a stirred solution of N—((R)-1-aminopropan-2-yl)-8-(4-(trifluoromethyl)cyclohex-1-en-1-yl)quinoline-3-carboxamide (Compound 53) (0.115 g, 0.30 mmol, 1.0 eq.) in DCM (2 mL) was added acetyl chloride (0.040 g, 0.36 mmol, 1.2 eq.) under N₂ atmosphere. Followed by addition of TEA (0.08 mL, 0.61 mmol, 2.0 eq.) and reaction mixture was stirred at RT for 1 h. TLC indicated the completion of reaction, the resulting reaction mixture was diluted with water (10 mL) and extracted with DCM (3×20 mL). The combined organic layers were dried over Na₂SO₄ and concentrated under reduced pressure to afford crude. The crude was purified by manual column chromatography using silica gel (230-400 mesh) as stationary phase (gradient elution 20% Ethyl acetate in hexane) to afford N—((R)-1-acetamidopropan-2-yl)-8-(4-(trifluoromethyl)cyclohex-1-en-1-yl)quinoline-3-carboxamide (Compound 70) (0.053 g, 0.128 mmol, Yield 41.47%).

¹H NMR (DMSO-d₆, 400 MHz): δ ppm 9.24 (d, J=2.0 Hz, 1H), 8.78 (d, J=2.0 Hz, 1H), 8.56 (d, J=8.0 Hz, 1H), 8.02-7.99 (m, 2H), 7.64-7.63 (m, 2H), 5.87 (br s, 1H), 4.15-4.11 (m, 1H), 3.30-3.17 (m, 2H), 2.79-2.60 (m, 3H), 2.32-2.25 (m, 1H), 2.10-2.07 (m, 1H), 1.82 (s, 3H), 1.73-1.67 (m, 1H), 1.15 (d, J=6.8 Hz, 3H). Note: CF₃CH proton merged with DMSO solvent peak.

¹H NMR (MeOD, 400 MHz): δ ppm 9.23 (d, J=2.0 Hz, 1H), 8.74 (d, J=2.0 Hz, 1H), 7.98 (dd, J=5.6, 2.0 Hz, 1H), 7.69-7.63 (m, 2H), 5.86 (br s, 1H), 4.35-4.30 (m, 1H), 3.47-3.32 (m, 2H), 2.78-2.68 (m, 3H), 2.65-2.55 (m, 1H), 2.51-2.40 (m, 1H), 2.17-2.09 (m, 1H), 1.97 (s, 3H), 1.39-1.29 (m, 1H), 0.93 (d, J=6.8 Hz, 3H). Note: —CONH protons exchanged in MeOD NMR.

LCMS (Method A): 2.22 min, 100%, 220 nm, MS: ES+420.13 (M+1)

HPLC (Method A): 7.75 min, 99.80%, 210 nm

Chiral HPLC: 6.74 min, 7.06 min, 48.93%, 51.06%, 240 nm

### Example 67—Chiral Separation of N—((S)-1-(pyridin-2-yl)ethyl)-8-(4-(trifluoromethyl)cyclohex-1-en-1-yl)quinoline-3-carboxamide (Compound 71 and Compound 72)

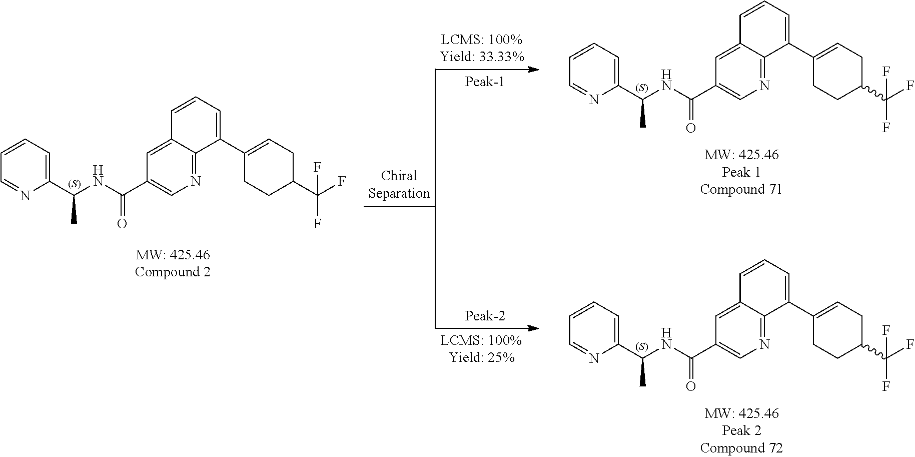

COLUMN ID: CHIRALCEL ODH (250×4.6 mm 5 um)

MOBILE PHASE A: Liq. CO₂

MOBILE PHASE B: M·NH3-MEOH-ACN (50-50)

FLOW RATE (ML/MIN): 3

INSTRUMENT ID: SFC INVESTIGATOR

METHOD: TIME: FLOW: % A: % B (0:14:60:40), (7:14:60:40)

Input Quantity: 0.060 g.

Output Quantity: Compound 71=0.015 g (% Yield=33.33%) and Compound 72=0.013 g (% Yield=25%)

### Compound 71:

¹H NMR (MeOD, 400 MHz): δ ppm, 9.27 (d, J=2.4 Hz, 1H), 8.86 (d, J=2 Hz, 1H), 8.56 (d, J=4.8 Hz, 1H), 7.99 (dd, J=7.6, 1.6 Hz, 1H), 7.86 (td, J=7.6, 1.6 Hz, 1H), 7.70-7.64 (m, 2H), 7.55 (d, J=8 Hz, 1H), 7.36-7.33 (m, 1H), 5.87 (s, 1H), 5.36-5.32 (m, 1H), 2.78-2.65 (m, 3H), 2.55-2.51 (m, 1H), 2.40-2.37 (m, 1H), 2.18-2.15 (m, 1H), 1.92-1.85 (m, 1H), 1.66 (d, J=6.8 Hz, 3H). Note: —CONH proton exchanged in MeOD NMR.

¹H NMR (DMSO-d₆, 400 MHz): δ ppm, 9.29 (d, J=2.4 Hz, 1H), 9.21 (d, J=7.6 Hz, 1H), 8.89 (d, J=2.0 Hz, 1H), 8.54 (d, J=4.4 Hz, 1H), 8.02 (dd, J=5.2, 4.0 Hz, 1H), 7.80-7.75 (m, 1H), 7.65-7.62 (m, 2H), 7.48 (d, J=8.0 Hz, 1H), 7.29 (dd, J=7.2, 5.2 Hz, 1H), 5.87 (br s, 1H), 5.29-5.22 (m, 1H), 2.81-2.67 (m, 3H), 2.33-2.29 (m, 1H), 2.10-2.07 (m, 1H), 1.74-1.65 (m, 1H), 1.55 (d, J=7.2 Hz, 3H).

LCMS (Method A): 2.321 min, 100%, 254.0 nm, MS: ES+426.3 (M+1)

HPLC (Method A): 9.18 min, 100%, 254.0 nm

Chiral HPLC: 2.29 min, 100%, 246 nm

### Compound 72:

¹H NMR (MeOD, 400 MHz): δ ppm, 9.27 (d, J=2.4 Hz, 1H), 8.86 (d, J=2 Hz, 1H), 8.56 (d, J=4.8 Hz, 1H), 7.99 (dd, J=7.6, 1.6 Hz, 1H), 7.86 (td, J=7.6, 1.6 Hz, 1H), 7.70-7.64 (m, 2H), 7.55 (d, J=8 Hz, 1H), 7.36-7.33 (m, 1H), 5.87 (s, 1H), 5.36-5.32 (m, 1H), 2.78-2.65 (m, 3H), 2.55-2.51 (m, 1H), 2.40-2.37 (m, 1H), 2.18-2.15 (m, 1H), 1.92-1.85 (m, 1H), 1.66 (d, J=6.8 Hz, 3H). Note: —CONH proton exchanged in MeOD NMR.

¹H NMR (DMSO-d₆, 400 MHz): δ ppm, 9.29 (d, J=2.4 Hz, 1H), 9.21 (d, J=7.6 Hz, 1H), 8.89 (d, J=2.0 Hz, 1H), 8.54 (d, J=4.4 Hz, 1H), 8.02 (dd, J=5.2, 4.0 Hz, 1H), 7.80-7.75 (m, 1H), 7.65-7.62 (m, 2H), 7.48 (d, J=8.0 Hz, 1H), 7.29 (dd, J=7.2, 5.2 Hz, 1H), 5.87 (br s, 1H), 5.29-5.22 (m, 1H), 2.81-2.67 (m, 3H), 2.33-2.29 (m, 1H), 2.10-2.07 (m, 1H), 1.74-1.65 (m, 1H), 1.55 (d, J=7.2 Hz, 3H).

LCMS (Method A): 2.321 min, 100%, 254.0 nm, MS: ES+426.3 (M+1)

HPLC (Method A): 9.20 min, 100%, 254.0 nm

Chiral HPLC (Method A): 2.32 min, 100%, 246 nm

### Example 68—Synthesis of N—((R)-1-(oxazol-2-yl)ethyl)-8-(4-(trifluoromethyl)cyclohex-1-en-1-yl)quinoline-3-carboxamide (Compound 73)

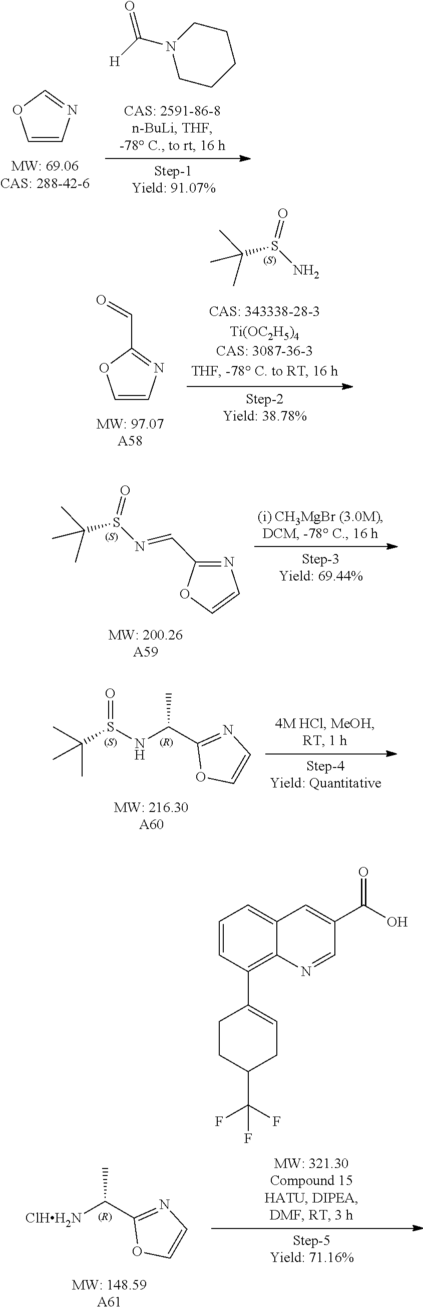

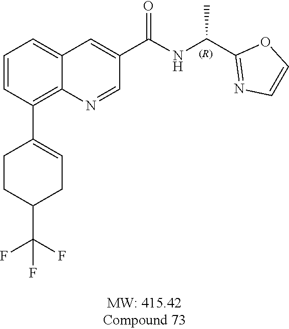

### Step-1

To a stirred solution of oxazole CAS: 288-42-6 (10.0 g, 144.80 mmol, 1.0 eq.) in dry THF (50 mL) was added n-BuLi (90.50 mL, 144.80 mmol, 1.0 eq.) at −78° C. and stirred for 1 h. Followed by addition of CAS: 2591-86-8 (16.36 g, 144.80 mmol, 1.0 eq.) under N₂ atmosphere. The resulting reaction mixture was stirred at RT for 16 h. TLC indicated the completion of reaction; the resulting reaction mixture was quenched with 1N HCl (200 mL) and extracted with DCM (3×300 mL). The combined organic layers were dried over Na₂SO₄ and concentrated under reduced pressure to afford crude oxazole-2-carbaldehyde (A58) (12.8 g, 131.86 mmol, Yield: 91.07%). The obtained crude material was directly used for next reaction (Note: organic layer was concentrated at lower temperature 25° C. on rotavapor due to volatile nature of the compound, also did not removed solvent completely)

¹H NMR (DMSO-d₆, 400 MHz): δ ppm, 9.73 (s, 1H), 8.50 (s, 1H), 7.66 (s, 1H).

### Step-2

To a solution of oxazole-2-carbaldehyde (A58) (5.0 g, 51.509 mmol, 1.0 eq.) in THF (50 mL), CAS: 343338-28-3 (7.49 g, 61.811 mmol, 1.2 eq.) and Ti(OC₂H₄)₄ CAS: 3087-36-3 (22.988 g, 103.06 mmol, 2.0 eq.) were added and allowed reaction mixture to stirred at RT for 16 h. TLC indicate the completion of reaction, the resulting reaction mixture was quenched with saturated brine (100 mL) and extracted with EtOAc (3×300 mL). The combined organic layers were dried over Na₂SO₄ and concentrated under reduced pressure to afford crude. The obtained crude material was purified by flash column chromatography using silica (100-200 mesh) as stationary phase (desired product was eluted in 40% Ethyl acetate in hexane) to afford (S,E)-2-methyl-N-(oxazol-2-ylmethylene) propane-2-sulfinamide (A59) (4.0 g, 19.974 mmol, Yield: 38.78%).

¹H NMR (DMSO-d₆, 400 MHz): δ ppm, 8.46 (s, 1H), 8.29 (s, 1H), 7.64 (s, 1H), 1.19 (s, 9H).

LCMS (Method A): 1.547 min, 100%, 285.0 nm, MS: ES+201.0 (M+1)

### Step-3

To a stirred solution of (E)-2-methyl-N-(oxazol-2-ylmethylene) propane-2-sulfinamide (A59) (4.0 g, 19.974 mmol, 1.0 eq.), in DCM (40 mL) was added CH₃MgBr (3M in Et₂O) (7.989 mL, 23.969 mmol, 1.2 eq.) under N₂ at −78° C. The resulting mixture was stirred at −78° C. for 1.5 h, then allowed to warm up to room temperature for 1.5 h. The second portion of CH₃MgBr (3M in Et₂O) (7.989 mL, 23.969 mmol, 1.2 eq.) was added and again stirred reaction mixture at −78° C. for 1 h. After 1 h allowed to warm up to room temperature for 3 h. The third portion of CH₃MgBr (3M in Et₂O) (3.99 mL, 11.984 mmol, 0.6 eq.) was added at −78° C., then allowed reaction mixture to warm up to room temperature for 16 h. TLC indicated the completion of reaction, the resulting reaction mixture was poured into aq. NH₄Cl (100 mL) slowly and extracted with EtOAc (3×100 mL). The combined organic layers were dried over Na₂SO₄ and concentrated under reduced pressure to afford crude. The obtained crude material was purified by trituration with pentane to afford (S)-2-methyl-N—((R)-1-(oxazol-2-yl)ethyl) propane-2-sulfinamide (A60) (3.0 g, 13.868 mmol, Yield: 69.44%).

¹H NMR (DMSO-d₆, 400 MHz): 8.07 (s, 1H), 7.16 (s, 1H), 5.93 (d, J=7.6 Hz, 1H), 4.56-4.48 (m, 1H), 1.50 (d, J=6.8 Hz, 3H), 1.09 (s, 9H).

LCMS (Method A): 1.404 min, 100%, 220.0 nm, MS: ES+217.1 (M+1).

Chiral HPLC: 4.14 min, 100%, 216.0 nm.

### Step-4

To a solution of(S)-2-methyl-N—((R)-1-(oxazol-2-yl)ethyl) propane-2-sulfinamide (A60) (3.0 g, 13.870 mmol, 1.0 eq.) in MeOH (30 mL) was added 4M HCl in Dioxane (7.28 mL, 29.126 mmol, 2.1 eq.) and stirred for 1 h at RT. TLC indicated the completion of reaction then concentrated under reduced pressure to afford (R)-1-(oxazol-2-yl)ethan-1-amine hydrochloride (A61) (2.77 g, 18.642 mmol, Yield: Quantitative)

¹H NMR (DMSO-d₆, 400 MHz): δ ppm, 8.78 (br s, 2H), 8.25 (s, 1H), 7.33 (s, 1H), 4.70-4.64 (m, 1H), 1.56 (d, J=6.8 Hz, 3H).

LCMS (Method A): 0.196 min, 94.57%, 210.0 nm, MS: ES+112.9 (M+1)

### Step-5

To a solution of 8-(4-(trifluoromethyl)cyclohex-1-en-1-yl)quinoline-3-carboxylic acid Compound 15 (5.0 g, 15.562 mmol, 1.0 eq.), HATU (8.876 g, 23.343 mmol, 1.5 eq.) and DIPEA (8.045 MI, 46.685 mmol, 3.0 eq.) in DMF (50 Ml) at 0° C. under nitrogen atmosphere stirred for 20 min, was added A61 (2.77 g, 18.674 mmol, 1.2 eq.) and reaction mixture was stirred for 3 h at room temperature. TLC indicated the completion of reaction, then water (100 mL) was added and extracted with EtOAc (3×100 mL). The combined organic layers were dried over Na₂SO₄ and concentrated under reduced pressure to afford crude. The obtained crude material was purified by flash column chromatography using silica gel (230-400 mesh) as stationary phase (desired product eluted in 60% Ethyl acetate in hexane) to afford N—((R)-1-(oxazol-2-yl)ethyl)-8-(4-(trifluoromethyl)cyclohex-1-en-1-yl)quinoline-3-carboxamide (Compound 73) (4.6 g, 11.073 mmol, Yield: 71.16%).

¹H NMR (DMSO-d₆, 400 MHz): δ ppm, 9.36 (d, J=7.6 Hz, 1H), 9.27 (d, J=2.0 Hz, 1H), 8.86 (d, J=2.0 Hz, 1H), 8.08 (s, 1H), 8.03-8.00 (m, 1H), 7.66-7.63 (m, 2H), 7.19 (s, 1H), 5.88 (br s, 1H), 5.40-5.36 (m, 1H), 2.80-2.67 (m, 4H), 2.33-2.29 (m, 1H), 2.10-2.07 (m, 1H), 1.73-1.65 (m, 1H), 1.61 (d, J=6.8 Hz, 3H).

¹H NMR (MeOD, 400 MHz): δ ppm, 9.27 (d, J=2.0 Hz, 1H), 8.83 (d, J=2.0 Hz, 1H), 7.99-7.92 (m, 2H), 7.70-7.63 (m, 2H), 7.18 (s, 1H), 5.88 (br s, 1H), 5.52-5.46 (m, 1H), 2.78-2.75 (m, 1H), 2.68-2.65 (m, 2H), 2.55-2.51 (m, 1H), 2.40-2.37 (m, 1H), 2.18-2.15 (m, 1H), 1.91-1.85 (m, 1H), 1.73 (d, J=6.8 Hz, 3H). Note: —CONH proton exchanged in MeOD NMR.

LCMS (Method A): 2.460 min, 98.73%, 254.0 nm, MS: ES+416.2 (M+1)

HPLC (Method A): 8.77 min, 99.01%, 254.0 nm.

Chiral HPLC: Peak-1 (5.02 min, 47.83%, 245.0 nm); Peak-2 (6.08 min, 49.16%, 245.0 nm)

### Example 69—Synthesis of N—((R)-4-(dimethylamino)-4-oxobutan-2-yl)-8-(4-(trifluoromethyl)cyclohex-1-en-1-yl)quinoline-3-carboxamide (Compound 74)

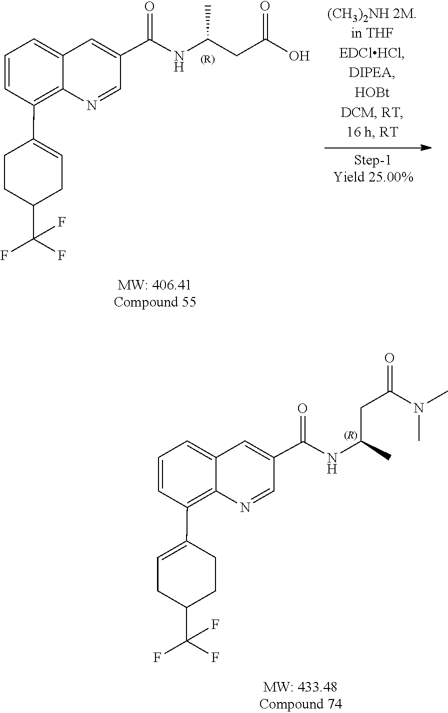

### Step-1

To a solution of (3R)-3-(8-(4-(trifluoromethyl)cyclohex-1-en-1-yl)quinoline-3-carboxamido)butanoic acid (Compound 55) (0.15 g, 0.36 mmol, 1.0 eq.), EDCl·HCl (0.106 g, 0.553 mmol, 1.5 eq.), HOBt (0.075 g, 0.553 mmol, 1.5 eq.) and DIPEA (0.143 g, 1.107 mmol, 3.0 eq.) in DCM (20 v) at 0° C. under nitrogen atmosphere stirred for 15 min, was added Dimethyl amine (2 M in THF) CAS: 124-40-3 (0.067 g, 1.476 mmol, 4.0 eq.) under Nitrogen. The resulting mixture was stirred for 16 h at room temperature. TLC indicated the completion of reaction, the resulting reaction mixture was quenched with ice-cold water (50 mL) and extracted with DCM (3×15 mL). The combined organic layers were dried over Na₂SO₄ and concentrated under reduced pressure to afford crude. The obtained crude material was purified by trituration with n-pentane to afford N—((R)-4-(dimethylamino)-4-oxobutan-2-yl)-8-(4-(trifluoromethyl)cyclohex-1-en-1-yl)quinoline-3-carboxamide (Compound 74) (0.04 g, 0.0922 mmol, Yield: 25%).

¹H NMR (MeOD, 400 MHz) δ ppm 9.22 (d, J=2.4 Hz, 1H), 8.74 (d, J=2.4 Hz, 1H), 7.95 (dd, J=7.6, 2.0 Hz, 1H), 7.68-7.65 (m, 2H), 5.87 (br s, 1H), 4.62-4.57 (m, 1H), 3.17 (s, 3H), 2.96 (s, 3H), 2.88-2.83 (m, 1H), 2.77-2.75 (m, 1H), 2.69-2.64 (m, 3H), 2.55-2.51 (m, 1H), 2.40-2.38 (m, 1H), 2.18-2.15 (m, 1H), 1.92-1.85 (m, 1H), 1.41 (d, J=5.4 Hz, 3H). Note: —CONH proton exchanged in MeOD NMR.

¹H NMR (DMSO-d6, 400 MHz) δ ppm 9.23 (d, J=2.4 Hz, 1H), 8.77 (d, J=2.4 Hz, 1H), 8.64 (d, J=7.6 Hz, 1H), 8.02-7.99 (m, 1H), 7.65-7.63 (m, 2H), 5.88 (br s, 1H), 4.42-4.39 (m, 1H), 3.02 (s, 3H), 2.83 (s, 3H), 2.76-2.71 (m, 2H), 2.69-2.61 (m, 2H), 2.57-2.53 (m, 1H), 2.51-2.43 (m, 1H), 2.35-2.32 (m, 1H), 2.07 (m, 1H), 1.75-1.67 (m, 1H), 1.25 (d, J=6.4 Hz, 3H).

LCMS (Method A): 2.290 min, 100%, 210.0 nm, MS: ES+434.3 (M+1)

HPLC (Method A): 8.26 min, 98.29%, 210.0 nm.

Chiral HPLC (Method A): Peak 1: 8.12 min, 45.66%, 240 nm; Peak 2: 8.49 min, 53.04%, 240 nm.

### Example 70—Synthesis of N-(3-(dimethylamino)-3-oxopropyl)-8-(4-(trifluoro methyl)cyclohex-1-en-1-yl)quinoline-3-carboxamide (Compound 75)

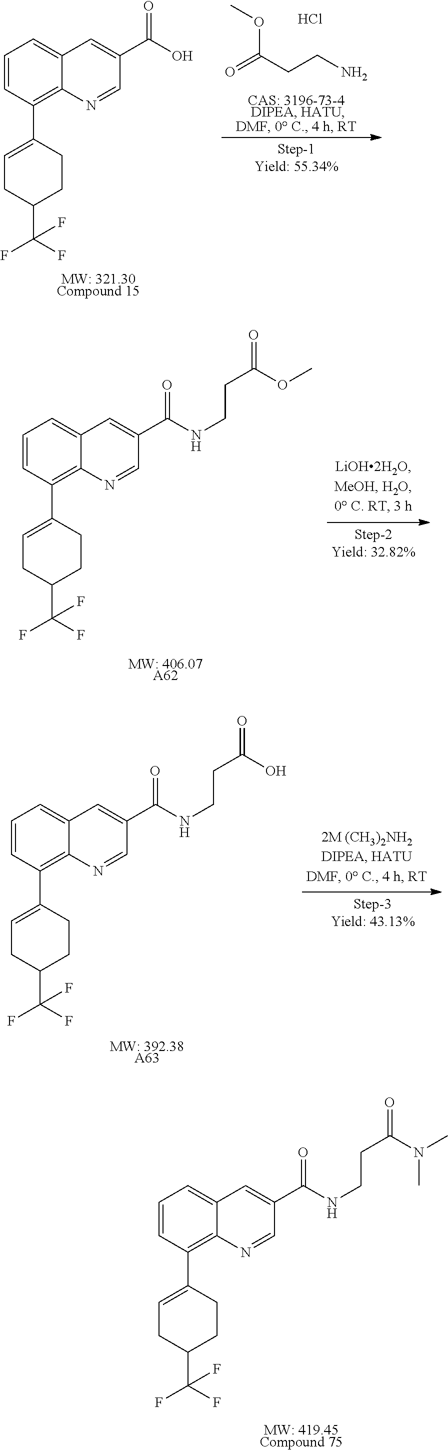

### Step-1

To a stirred solution of 8-(4-(trifluoromethyl)cyclohex-1-en-1-yl)quinoline-3-carboxylic acid (Compound 15) (0.5 g, 1.55 mmol, 1.0 eq.) in DMF (5.0 mL) was added HATU (0.88 g, 2.33 mmol, 1.5 eq.) at 0° C. Followed by addition of DIPEA (0.8 mL, 4.67 mmol, 3.0 eq.) and stirred at 0° C. After 10 min of stirring, CAS: 3196-73-4 B-Alanine methyl ester hydrochloride (0.239 g, 1.71 mmol, 1.1 eq.) was added at 0° C. The resulting reaction mixture was stirred at RT for 4 h. TLC indicated the completion of reaction; the resulting reaction mixture was diluted with ice-cold water (10 mL) and was extracted with EtOAc (20 mL). The combined organic layer was evaporated under vacuum and crude residue was purified with silica column chromatography (60-120 mesh; 50% of EtOAc and hexane) and yielded methyl 3-(8-(4-(trifluoromethyl)cyclohex-1-en-1-yl)quinoline-3-carboxamido) propanoate (A62) (0.34 g, 0.862 mmol, Yield: 55.34%).

¹H NMR (DMSO-d₆, 400 MHz): δ ppm 9.23 (d, J=2.0 Hz, 1H), 8.92 (t, J=6.8 Hz, 1H), 8.78 (d, J=1.6 Hz, 1H), 8.01-7.99 (m, 1H), 7.64-7.61 (m, 2H), 5.86 (br s, 1H), 3.62 (s, 3H), 3.57 (q, J=5.6 Hz, 2H), 2.80-2.64 (m, 5H), 2.45-2.40 (m, 1H), 2.31-2.24 (m, 1H), 2.09-2.06 (m, 1H), 1.74-1.63 (m, 1H).

LCMS (Method A): 2.418 min, 99.31%, MS: ES+407.22 [M+H].

### Step-2

To a solution of methyl 3-(8-(4-(trifluoromethyl)cyclohex-1-en-1-yl)quinoline-3-carboxamido) propanoate (A62) (0.340 g, 0.832 mmol) in MeOH:H₂O (3:1 mL) was added LiOH·H₂O (0.068 g, 1.67 mmol, 2 eq.) at 0° C. The reaction mixture was stirred for 3 h at room temperature, TLC indicate the completion of reaction, the resulting reaction mixture was acidified with 1N HCl and extracted with Ethyl Acetate (15 mL×3). The combined organic layer was concentrated under reduced pressure to afford 3-(8-(4-(trifluoromethyl)cyclohex-1-en-1-yl)quinoline-3-carboxamido) propanoic acid as a white amorphous solid (A63) (0.32 g, 0.765 mmol, Yield: 32.82%).

¹H NMR (DMSO-d₆, 400 MHz): δ ppm 9.24 (d, J=2.4 Hz, 1H), 8.92 (t, J=5.2 Hz, 1H), 8.81 (d, J=2.0 Hz, 1H), 8.02-7.99 (m, 1H), 7.65-7.62 (m, 2H), 5.87 (br s, 1H), 3.53 (q, J=6.8 Hz, 2H), 2.80-2.67 (m, 3H), 2.59-2.56 (m, 2H), 2.50-2.46 (m, 1H), 2.32-2.25 (m, 1H), 2.10-2.07 (m, 1H), 1.74-1.65. (m, 1H).

LCMS (Method A): 2.191 min, 100%, MS: ES+393.17 [M+H]⁺

### Step-3

To a stirred solution of 3-(8-(4-(trifluoromethyl)cyclohex-1-en-1-yl)quinoline-3-carboxamido) propanoic acid (A63) (0.15 g, 0.38 mmol, 1.0 eq.) in DMF (1.5 Ml) was added HATU (0.21 g, 0.57 mmol, 1.5 eq.) at 0° C. Then after 10 min at same temperature DIPEA (0.2 MI, 1.14 mmol, 3.0 eq.) was added and stirred for 10 min. After 10 min of stirring, dimethyl amine (2M in THF) CAS: 124-40-3 (0.018 g, 0.42 mmol, 1.1 eq.) was added at 0° C. The resulting reaction mixture was stirred at RT for 4 h. TLC indicated the completion of reaction; the resulting reaction mixture was diluted with ice-cold water (10 Ml) and was extracted with EtOAc (10 Ml×2). The combined layer was evaporated under vacuum and crude residue was purified by column chromatography using silica gel (60-120 mesh; 50% of EtOAc and hexane) to afford N-(3-(dimethylamino)-3-oxopropyl)-8-(4-(trifluoromethyl)cyclohex-1-en-1-yl)quinoline-3-carboxamide (Compound 75) (0.064 g, 0.155 mmol, Yield: 43.13%).

¹H NMR (DMSO-d₆, 400 MHz): δ ppm 9.24 (d, J=2.0 Hz, 1H), 8.84 (t, J=5.2 Hz, 1H), 8.78 (d, J=2.4 Hz, 1H), 8.01-7.98 (m, 1H), 7.64-7.61 (m, 2H), 5.86 (br s, 1H), 3.52 (q, J=5.6 Hz, 2H), 2.97 (s, 3H), 2.88-2.77 (m, 5H), 2.71-2.62 (m, 3H), 2.32-2.24 (m, 1H), 2.09-2.07 (m, 1H), 1.74-1.63 (m, 1H). Note: CF₃CH proton merged with DMSO solvent peak.

¹H NMR (MeOD, 400 MHz): δ ppm 9.24 (d, J=2.0 Hz, 1H), 8.74 (d, J=2.4 Hz, 1H), 7.96 (dd, J=1.6 Hz, 7.6 Hz, 1H), 7.68-7.63 (m, 2H), 5.86 (br s, 1H), 3.73 (t, J=6.8 Hz, 2H), 3.11 (s, 3H), 2.98 (s, 3H), 2.80 (t, J=6.8 Hz, 3H), 2.75-2.64 (m, 2H), 2.55-2.50 (m, 1H), 2.40-2.37 (m, 1H), 2.17-2.15 (m, 1H), 1.92-1.88 (m, 1H). Note: —CONH proton exchanged in MeOD NMR.

LCMS (Method A): 2.22 min, 99.25%, MS: ES+420.3 [M+H]

HPLC (Method A): 7.98 min. 99.11%, 254 nm

CHIRAL HPLC (Method A): 6.46+7.39 min. 49.63+49.80%, 242 nm

### Example 71—Synthesis of N-(3-(methylamino)-3-oxopropyl)-8-(4-(trifluoro methyl)cyclohex-1-en-1-yl)quinoline-3-carboxamide (Compound 76)

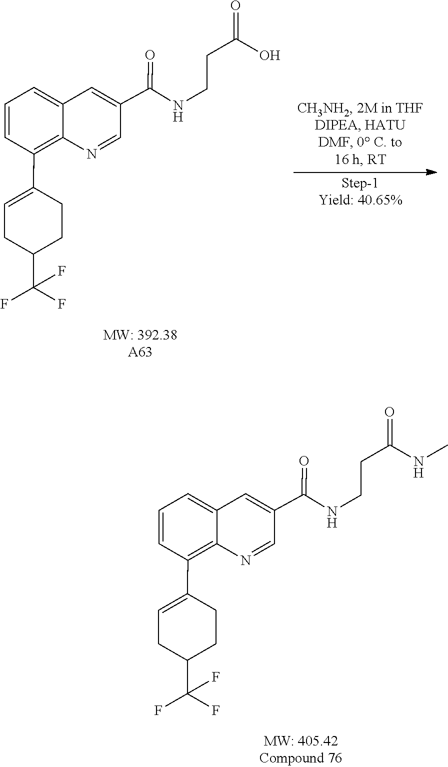 [SMILES: O=C(O)CCNC(=O)c1cnc2c(C3=CCC(C(F)(F)F)CC3)cccc2c1 ; CNC(=O)CCNC(=O)c1cnc2c(C3=CCC(C(F)(F)F)CC3)cccc2c1]

### Step-1

To a stirred solution of 3-(8-(4-(trifluoromethyl)cyclohex-1-en-1-yl)quinoline-3-carboxamido) propanoic acid A63 (0.15 g, 0.38 mmol, 1.0 eq.) in DMF (1.5 mL) was added HATU (0.21 g, 0.57 mmol, 1.5 eq.) at 0° C. Followed by addition of DIPEA (0.2 mL, 1.14 mmol, 3.0 eq.) and CAS: 74-89-5 methyl amine 2M in THF (0.28 mL, 0.57 mmol, 1.5 eq.). The resulting reaction mixture was stirred at RT for 16 h. TLC indicated the completion of reaction, the resulting reaction mixture was diluted with ice-cold water (10 mL) and extracted with EtOAc (25 mL). The combined layer was evaporated under vacuum and crude residue was purified with silica column chromatography (60-120 mesh; 50% of EtOAc and hexane) to afford N-(3-(methylamino)-3-oxopropyl)-8-(4-(trifluoromethyl)cyclohex-1-en-1-yl)quinoline-3-carboxamide (Compound 76) (0.065 g, 0.155 mmol, Yield: 40.65%).

¹H NMR (DMSO-d₆, 400 MHz): δ ppm 9.23 (d, J=2.0 Hz, 1H), 8.88 (t, J=5.6 Hz, 1H), 8.78 (d, J=2.0 Hz, 1H), 8.01-7.98 (m, 1H), 7.88-7.87 (m, 1H), 7.64-7.61 (m, 2H), 5.86 (br s, 1H), 3.55 (q, J=7.2 Hz, 2H), 2.85-2.75 (m, 1H), 2.74-2.65 (m, 1H), 2.58 (d, J=4.8 Hz, 3H), 2.42-2.39 (m, 3H), 2.18-2.07 (m, 1H), 1.74-1.63 (m, 1H). Note: 3H proton merged in DMSO solvent peak.

¹H NMR (MeOD, 400 MHz): δ ppm 9.23 (d, J=2.0 Hz, 1H), 8.74 (d, J=2.4 Hz, 1H), 7.98-7.95 (m, 1H), 7.69-7.63 (m, 2H), 5.86 (br s, 1H), 3.72 (t, J=6.8 Hz, 2H), 2.75 (s, 3H), 2.68-2.64 (m, 2H), 2.57 (t, J=6.8 Hz, 2H), 2.51-2.49 (m, 1H), 2.40-2.34 (m, 1H), 2.18-2.15 (m, 1H), 1.89-1.85 (m, 1H). Note: —CONH proton exchanged in MeOD NMR.

LCMS (Method A): 2.12 min, 98.87%, MS: ES+406.37 [M+H]

HPLC (Method A): 7.49 min. 99.45%, 254 nm

### Example 72—Synthesis of N—((R)-4-acetamidobutan-2-yl)-8-(4-(trifluoromethyl)cyclohex-1-en-1-yl)quinoline-3-carboxamide (Compound 77)

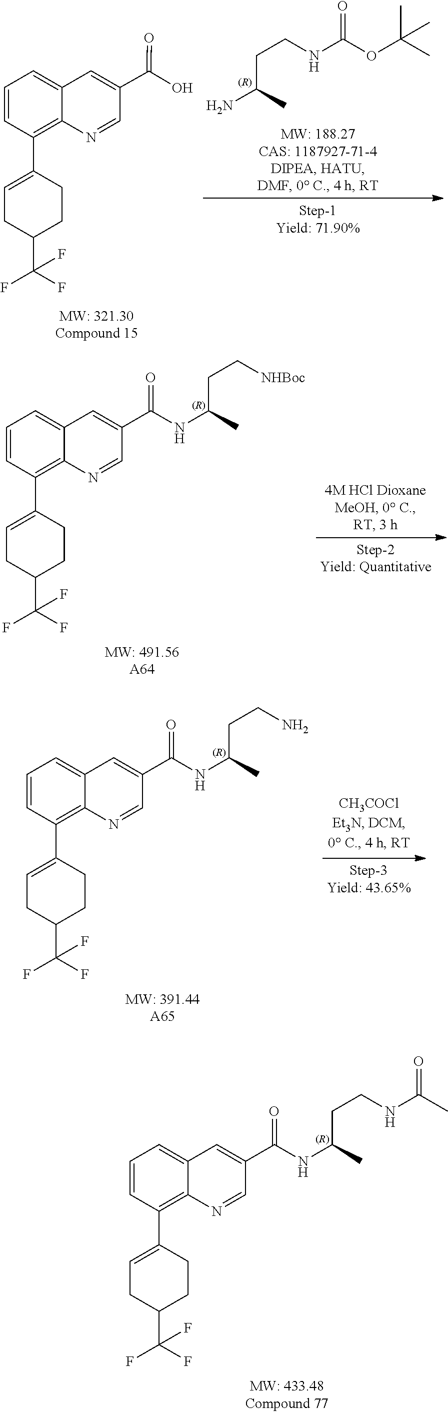

### Step-1

To a solution of 8-(4-(trifluoromethyl)cyclohex-1-en-1-yl)quinoline-3-carboxylic acid (Compound 15) (0.2 g, 0.622 mmol, 1.0 eq.) in DMF (3 mL) was added HATU (0.355 g, 0.933 mmol, 1.5 eq.) and DIPEA (0.32 mL, 1.867 mmol, 3.0 eq.) at 0° C. under nitrogen atmosphere then stirred for 5 min, followed by addition of CAS: 1187927-71-4 (R)-1-Boc-amino-butyl-3-amine (0.129 g, 0.685 mmol, 1.1 eq.) under Nitrogen. The resulting mixture was stirred for 4 h at room temperature. TLC indicated the completion of reaction, the resulting reaction mixture was quenched with ice-cold water (10 mL) and extracted with DCM (3×20 mL). The combined organic layers were dried over Na₂SO₄ and concentrated under reduced pressure to afford crude. The obtained crude material was purified by flash column chromatography using silica (230-400 Mesh) as stationary phase (50% EtOAc in Hexane) to afford tert-butyl ((3R)-3-(8-(4-(trifluoromethyl)cyclohex-1-en-1-yl)quinoline-3-carboxamido)butyl) carbamate (A64) (0.22 g, 0.448 mmol, Yield: 71.90%).

¹H NMR (DMSO-d₆, 400 MHz): 9.24 (d, J=2.0 Hz, 1H), 8.78 (d, J=2.0 Hz, 1H), 8.55 (d, J=8.0 Hz, 1H), 8.00 (t, J=4.8 Hz, 1H), 7.65-7.63 (m, 2H), 6.80 (br s, 1H), 5.87 (br s, 1H), 4.11-4.08 (m, 1H), 3.02-2.97 (m, 2H), 2.73-2.68 (m, 3H), 2.33-2.25 (m, 2H), 2.09-2.07 (m, 1H), 1.73-1.65 (m, 3H), 1.36 (s, 9H), 1.20 (d, J=6.8 Hz, 3H).

LCMS (Method A): 2.725 min, 96.87%, 254.0 nm, MS: ES+492.4 (M+1).

### Step-2:

To a solution of tert-butyl ((3R)-3-(8-(4-(trifluoromethyl)cyclohex-1-en-1-yl)quinoline-3-carboxamido)butyl) carbamate (A64) (0.22 g, 0.448 mmol, 1.0 eq.) in MeOH (2 mL) was added 4 M HCl in Dioxane (0.22 mL, 0.895 mmol, 2.0 eq.) and stirred at RT for 3 h. TLC indicated the completion of reaction, the reaction mixture was concentrated under reduced pressure to afford the crude. The obtained crude material was purified by trituration with diethyl ether (5 mL) to afford N—((R)-4-aminobutan-2-yl)-8-(4-(trifluoromethyl)cyclohex-1-en-1-yl)quinoline-3-carboxamide (A65) (0.2 g, 0.511 mmol, Quantitative).

¹H NMR (DMSO-d₆, 400 MHz): δ ppm, 9.28 (s, 1H), 8.87 (s, 1H), 8.78 (d, J=8.0 Hz, 1H), 8.03 (t, J=5.2 Hz, 3H), 7.87 (br s, 2H), 7.67-7.64 (m, 2H), 5.88 (br s, 1H), 4.19-4.16 (m, 1H), 3.72-3.46 (m, 5H), 2.89-2.86 (m, 2H), 2.76-2.68 (m, 2H), 2.33-2.25 (m, 1H), 2.10-2.07 (m, 1H), 1.86-1.82 (m, 2H), 1.75-1.69 (m, 1H), 0.85 (d, J=6.8 Hz, 3H). Note: Compound was isolated as HCl salt.

LCMS (Method A): 1.853 min, 97.76%, 254.0 nm, MS: ES+392 (M+1)

### Step-3

To a solution N—((R)-4-aminobutan-2-yl)-8-(4-(trifluoromethyl)cyclohex-1-en-1-yl)quinoline-3-carboxamide (A65) (0.18 g, 0.460 mmol, 1.0 eq.) and TEA (0.2 mL, 1.379 mmol, 3.0 eq.) in DCM (2 mL) at 0° C. under nitrogen atmosphere stirred for 10 min, was added CH₃COCl (0.036 mL, 0.460 mmol, 1.0 eq.) and the reaction mixture was stirred for another 4 h at room temperature. TLC indicated the completion of reaction; the resulting reaction mixture was quenched with ice cold water (10 mL) and extracted with DCM (10 mL). The combined organic layers were dried over Na₂SO₄ and concentrated under reduced pressure to afford crude. The obtained crude material was purified by flash column chromatography using silica (230-400 Mesh) as stationary phase (50% EtOAc in Hexane) to afford N—((R)-4-acetamidobutan-2-yl)-8-(4-(trifluoromethyl)cyclohex-1-en-1-yl)quinoline-3-carboxamide (Compound 77) (0.087 g, 0.201 mmol, Yield: 43.65%).

¹H NMR (DMSO-d₆ 400 MHz): δ ppm, 9.24 (s, 1H), 8.78 (s, 1H), 8.58 (d, J=7.6 Hz, 1H), 8.01 (s, 1H), 7.85 (s, 1H), 7.63 (d, J=3.6 Hz, 2H), 5.87 (br s, 1H), 4.12-4.09 (m, 1H), 3.11-3.09 (m, 2H), 2.77-2.68 (m, 3H), 2.32-2.29 (m, 2H), 2.10-2.07 (m, 1H), 1.84 (s, 3H), 1.78-1.64 (m, 3H), 1.20 (d, J=6.4 Hz, 3H).

LCMS (Method A): 2.198 min, 98.97%, 242.0 nm, MS: ES+434.3 (M+1)

HPLC (Method A): 7.87 min, 97.93%, 254.0 nm

CHIRAL HPLC: 4.93 min. 98%, 242 nm

### Example 73—Synthesis of N-(3-acetamidopropyl)-8-(4-(trifluoromethyl)cyclohex-1-en-1-yl)quinoline-3-carboxamide (Compound 78)

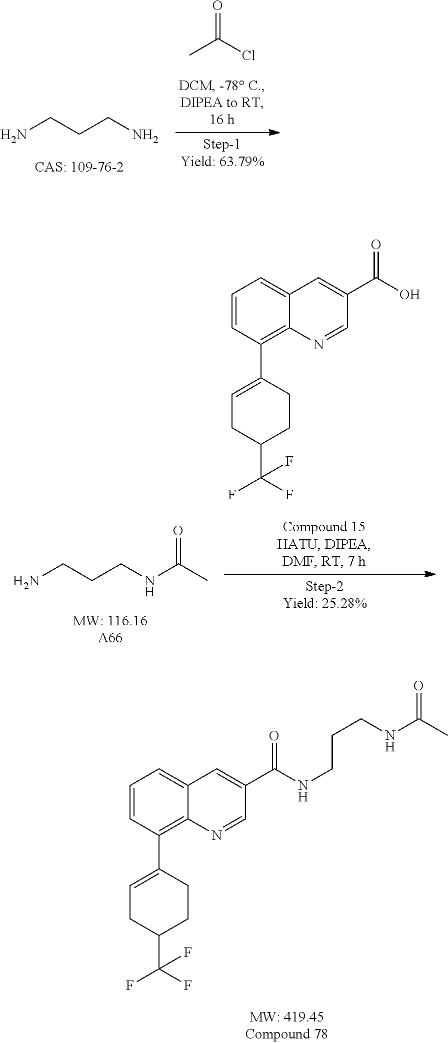

### Step-1

A solution of propane-1,3-diamine (CAS: 109-76-2) (0.1 g, 1.348 mmol, 1.0 eq.), CH₃COCl (0.105 g, 1.348 mmol, 1.0 eq.) and DIPEA (0.704 ml, 4.047 mmol, 3.0 eq.) in DCM (1 mL) at −78° C. under nitrogen atmosphere was stirred for 10 min then allowed to warm up to room temperature and stirring continued for 16 h at RT. TLC indicated the completion of reaction; the resulting reaction mixture was quenched with 5% HCl (2 mL) and basified with Sat. NaHCO₃ (3×10 mL) then extracted with DCM (10 mL) and the combined organic layers were dried over Na₂SO₄ and concentrated under reduced pressure to afford crude. The obtained crude material was purified by trituration with n-Pentane to afford N-(3-aminopropyl) acetamide (A66) (0.1 g, 0.862 mmol, Yield: 63.79%).

¹H NMR (DMSO-d₆, 400 MHz): δ ppm, 7.97-7.88 (m, 1H), 3.07-2.98 (m, 2H), 2.70 (t, J=6.8 Hz, 4H), 1.78 (s, 3H), 1.62-1.54 (m, 2H).

LCMS (Method B): 1.25 min, 78%, 210.0 nm, MS: ES+117 (M+1)

### Step-2

To a stirred solution of 8-(4-(trifluoromethyl)cyclohex-1-en-1-yl)quinoline-3-carboxylic acid (Compound 15) (0.1 g, 0.311 mmol, 1.0 eq.), HATU (0.17 g, 0.467 mmol, 1.5 eq.) and DIPEA (0.16 mL, 0.934 mmol, 3.0 eq.) in DMF (1 mL) at 0° C. under nitrogen atmosphere stirred for 10 min, was added A66 (0.039 g, 0.342 mmol, 1.1 eq.) and allowed to stirred for 7 h at room temperature. TLC indicated the completion of reaction, the resulting reaction mixture was quenched with ice-cold water (10 mL) and extracted with DCM (3×20 mL). The combined organic layers were dried over Na₂SO₄ and concentrated under reduced pressure to afford crude. The obtained crude material was purified by flash column chromatography using silica (230-400 Mesh) as stationary phase (5% MDC in MeOH) to afford N-(3-acetamidopropyl)-8-(4-(trifluoromethyl)cyclohex-1-en-1-yl)quinoline-3-carboxamide (Compound 78) (0.033 g, 0.078 mmol, Yield: 25.28%).

¹H NMR (MeOD, 400 MHz): δ ppm, 9.25 (d, J=2.0 Hz, 1H), 8.76 (d, J=2.0 Hz, 1H), 7.97 (dd, J=2.0 Hz J=7.6 Hz, 1H), 7.69-7.63 (m, 2H), 5.86 (br s, 1H), 3.51 (t, J=6.8 Hz, 2H), 2.78-2.75 (m, 1H), 2.68-2.65 (m, 2H), 2.55-2.51 (m, 1H), 2.40-2.33 (m, 1H), 2.17-2.14 (m, 1H), 1.98 (s, 3H), 1.92-1.82 (m, 3H). Note: —CONH proton exchanged in MeOD NMR.

¹H NMR (DMSO-d₆, 400 MHz): 9.24 (d, J=2.0 Hz, 1H), 8.80-8.78 (m, 1H), 8.02-7.99 (m, 1H), 7.90 (br s, 1H), 7.64-7.61 (m, 2H), 5.86 (br s, 1H), 3.12 (q, J=6.8 Hz, 2H), 2.80-2.67 (m, 3H), 2.33-2.32 (m, 1H), 2.10-2.07 (m, 1H), 1.81 (s, 3H), 1.76-1.64 (m, 3H). Note: CF₃CH proton merged in DMSO solvent peak.

LCMS (Method A): 2.150 min, 97.87%, 254.0 nm, MS: ES+420.38 (M+1)

HPLC (Method A): 7.62 min, 97.0%, 254.0 nm

### Example 74—Synthesis of N-(2-acetamidoethyl)-8-(4-(trifluoromethyl)cyclohex-1-en-1-yl)quinoline-3-carboxamide (Compound 79)

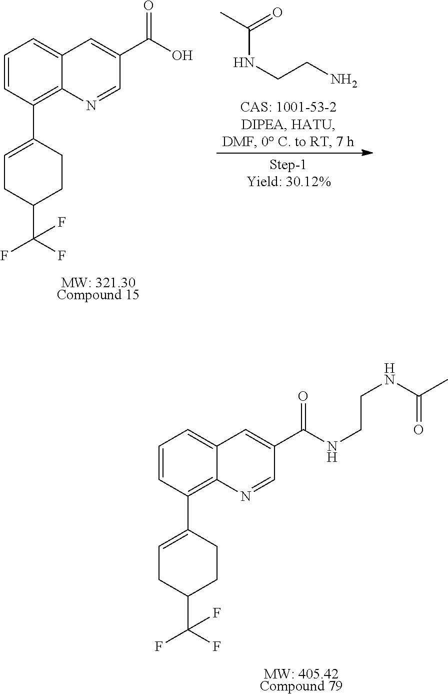

### Step-1

To a solution of 8-(4-(trifluoromethyl)cyclohex-1-en-1-yl)quinoline-3-carboxylic acid (Compound 15) (0.1 g, 0.311 mmol, 1.0 eq.) in DMF (1 mL) was added HATU (0.17 g, 0.467 mmol, 1.5 eq.) and DIPEA (0.16 mL, 0.934 mmol, 3.0 eq.) at 0° C. under nitrogen atmosphere and stirred for 10 min, was added CAS: 1001-53-2 N-Acetylethylenediamine (0.034 g, 0.342 mmol, 1.1 eq.) under Nitrogen. The resulting mixture was stirred for 7 h at room temperature. TLC indicated the completion of reaction, the resulting reaction mixture was quenched with ice-cold water (10 mL) and extracted with DCM (3×20 mL). The combined organic layers were dried over Na₂SO₄ and concentrated under reduced pressure to afford crude which was purified by flash column chromatography using silica (230-400 mesh) as stationary phase (5% MDC in MeOH) to afford N-(2-acetamidoethyl)-8-(4-(trifluoromethyl)cyclohex-1-en-1-yl)quinoline-3-carboxamide (Compound 79) (0.038 g, 0.093 mmol, Yield: 30.12%).

¹H NMR (MeOD, 400 MHz): δ ppm, 9.24 (d, J=2.0 Hz, 1H), 8.74 (d, J=2.4 Hz, 1H), 7.97 (dd, J=8.0 Hz, 7.6 Hz, 1H), 7.69-7.63 (m, 2H), 5.86 (br s, 1H), 3.59-3.56 (m, 2H), 3.48-3.45 (m, 2H), 2.78-2.75 (m, 1H), 2.68-2.65 (m, 2H), 2.55-2.51 (m, 1H), 2.40-2.37 (m, 1H), 2.17-2.15 (m, 1H), 1.98 (s, 3H), 1.89-1.85 (m, 1H). Note: —CONH proton exchanged in MeOD NMR.

¹H NMR (DMSO-d₆, 400 MHz): δ ppm, 9.24 (d, J=2.0 Hz, 1H), 8.86 (t, J=5.2 Hz, 1H), 8.79 (d, J=2.0 Hz, 1H), 8.04-7.98 (m, 2H), 7.64-7.61 (m, 2H), 5.87 (br s, 1H), 3.39-3.34 (m, 2H), 3.28-3.23 (m, 2H), 2.80-2.67 (m, 3H), 2.50-2.46 (m, 1H), 2.33-2.24 (m, 1H), 2.10-2.07 (m, 1H), 1.82 (s, 3H), 1.74-1.65 (m, 1H).

LCMS (Method A): 2.122 min, 100%, 242.0 nm, MS: ES+406.22 (M+1)

HPLC (Method A): 7.45 min, 99.69%, 254.0 nm

### Example 75—Synthesis of N-(1-(1H-pyrazol-5-yl)ethyl)-8-(4-(trifluoromethyl)cyclohex-1-en-1-yl)quinoline-3-carboxamide (Compound 80)

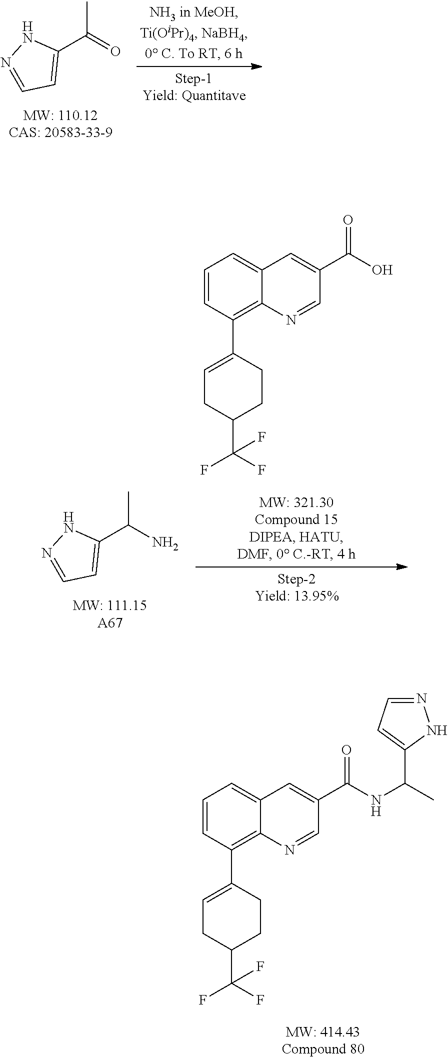

### Step-1

To a stirred solution of 1-(1H-pyrazol-5-yl)ethan-1-one (CAS: 20583-33-9) (0.2 g, 1.81 mmol, 1.0 eq.) in 7M NH₃ in MeOH (2.0 mL) was added Ti(Oⁱpr) 4 (1.0 g, 3.63 mmol, 2.0 eq.) at 0° C. and stirred for 4 h. Followed by addition of sodium borohydride (0.13 g, 3.63 mmol, 2.0 eq.) and stirred for 6 h at RT. TLC indicated the completion of reaction, the resulting reaction mixture was concentrated under vacuum to obtain crude product 1-(1H-pyrazol-5-yl)ethan-1-amine (A67) (0.22 g, 1.97 mmol, Yield: Quantitative).

LCMS (Method B): 1.22 min, 10.93%, 254 nm, MS: ES+112.2 (M+1)

### Step-2

To a stirred solution of 8-(4-(trifluoromethyl)cyclohex-1-en-1-yl)quinoline-3-carboxylic acid (Compound 15) (0.1 g, 0.31 mmol, 1.0 eq.) in DMF (1.0 mL) was added HATU (0.17 g, 0.46 mmol, 1.5 eq.) at 0° C. Followed by addition of DIPEA (0.16 mL, 0.934 mmol, 3.0 eq.) and stirred for another 10 min. After 10 min of stirring, 1-(1H-pyrazol-5-yl)ethan-1-amine (A67) (0.038 g, 0.342 mmol, 1.1 eq.) was added and reaction mixture was stirred at RT for 4 h. TLC indicated the completion of reaction; the resulting reaction mixture was poured on to cold water (5 mL) to obtain precipitate. The precipitate was filtered through buckler funnel and washed with water (20 mL) and dried over high vacuum then purified by column chromatography (desired product eluted in 80% EtOAC in Hexane) to afford N-(1-(1H-pyrazol-5-yl)ethyl)-8-(4-(trifluoromethyl)cyclohex-1-en-1-yl)quinoline-3-carboxamide (Compound 80) (0.018 g, 0.043 mmol, Yield: 13.95%).

¹H NMR (DMSO-d₆, 400 MHz): δ ppm, 12.57 (br s, 1H), 9.27 (d, J=2.0, 1H), 9.04 (br s, 1H), 8.84 (d, J=2.0 Hz, 1H), 8.01-7.98 (m, 1H), 7.64-7.63 (m, 3H), 6.25 (s, 1H), 5.87 (s, 1H), 5.39-5.31 (m, 1H), 2.81-2.67 (m, 2H), 2.50-2.46 (m, 2H), 2.33-2.29 (m, 1H), 2.10-2.07 (m, 1H), 1.73-1.67 (m, 1H), 1.54 (d, J=6.8 Hz, 3H).

LCMS (Method A): 2.293 min, 97.06%, 254 nm, MS: ES+415.2 (M+1)

HPLC (Method A): 8.25 min, 95.14%, 254 nm

### Example 76—Chiral separation of N—((R)-1-hydroxypropan-2-yl)-8-(4-(trifluoromethyl)cyclohex-1-en-1-yl)quinoline-3-carboxamide (Compound 81 and Compound 82)

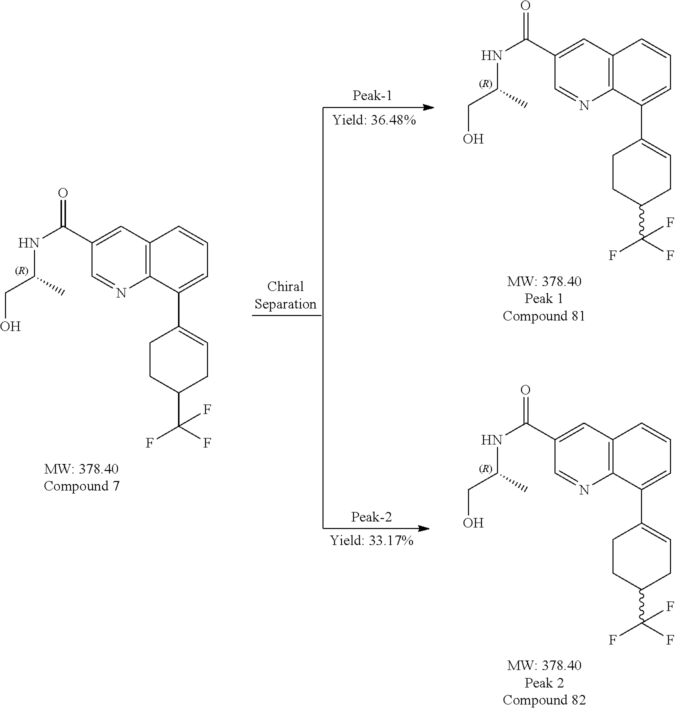

COLUMN ID: CHIRALPAK IG 250×50 mm 5 um

MOBILE PHASE A: LIQ. CO2

MOBILE PHASE B: MEOH

FLOW RATE (ML/MIN): 150

INSTRUMENT ID: WATERS SFC 350 WITH 2489 UV Detector

METHOD: TIME: FLOW: % A: % B (0.01:150:70:30), (24:150:70:30)

Input Quantity: 0.1206 g.

Output Quantity: Compound 81=0.044 g (% Yield=36.48%) and Compound 82=0.040 g (% Yield=33.17%)

### Compound 81:

¹H NMR (DMSO-d6, 400 MHz): δ ppm, δ 9.25 (d, J=2.0 Hz, 1H), 8.81 (d, J=2.0 Hz, 1H), 8.47 (d, J=7.6 Hz, 1H), 8.01-7.99 (m, 1H), 7.64-7.63 (m, 2H), 5.88 (br s, 1H), 4.79 (t, J=5.6 Hz, 1H), 4.10-4.07 (m, 1H), 3.53-3.49 (m, 1H), 3.42-3.37 (m, 1H), 2.73-2.68 (m, 3H), 2.33-2.29 (m, 1H), 2.10-2.07 (m, 1H), 1.72-1.67 (m, 1H), 1.18 (d, J=6.8 Hz, 3H).

¹H NMR (MeOD, 400 MHz): δ ppm, δ 9.25 (d, J=2.4 Hz, 1H), 8.78 (d, J=2.4 Hz, 1H), 7.97 (dd, J=7.6, 2.0 Hz, 1H), 7.69-7.63 (m, 2H), 5.87 (br s, 1H), 4.31-4.26 (m, 1H), 3.71-3.68 (m, 2H), 2.78-2.65 (m, 3H), 2.56-2.51 (m, 1H), 2.41-2.34 (m, 1H), 2.18-2.15 (m, 1H), 1.93-1.83 (m, 1H), 1.32 (d, J=6.8 Hz, 3H).

Note: —CONH and —OH proton exchanged in MeOD NMR.

LCMS (Method A): 2.212 min, 99.64%, 254.0 nm, MS: ES+379.2 (M+1) HPLC (Method A): 7.88 min, 99.59%, 254.0 nm

Chiral HPLC (Method A): 5.88 min, 99.76%, 240 nm

### Compound 82:

¹H NMR (DMSO-d6, 400 MHz): δ ppm, δ 9.25 (d, J=2.0 Hz, 1H), 8.81 (d, J=2.4 Hz, 1H), 8.47 (d, J=7.6 Hz, 1H), 8.01-7.99 (m, 1H), 7.64-7.63 (m, 2H), 5.87 (br s, 1H), 4.79 (t, J=5.6 Hz, 1H), 4.10-4.07 (m, 1H), 3.52-3.48 (m, 1H), 3.42-3.38 (m, 1H), 2.81-2.68 (m, 3H), 2.33-2.29 (m, 2H), 2.10-2.07 (m, 1H), 1.72-1.67 (m, 1H), 1.18 (d, J=6.8 Hz, 3H).

¹H NMR (MeOD, 400 MHz): δ ppm, δ 9.25 (d, J=2.0 Hz, 1H), 8.78 (d, J=2.4 Hz, 1H), 7.97 (dd, J=7.6, 2.0 Hz, 1H), 7.69-7.63 (m, 2H), 5.87 (br s, 1H), 4.31-4.26 (m, 1H), 3.71-3.68 (m, 2H), 2.78-2.65 (m, 3H), 2.56-2.51 (m, 1H), 2.41-2.34 (m, 1H), 2.18-2.15 (m, 1H), 1.93-1.82 (m, 1H), 1.32 (d, J=6.8 Hz, 3H).

Note: —CONH and —OH proton exchanged in MeOD NMR.

LCMS (Method A): 2.214 min, 100%, 254.0 nm, MS: ES+379.2 (M+1)

HPLC (Method A): 7.87 min, 98.49%, 254.0 nm

Chiral HPLC (Method A): 6.79 min, 98.70%, 240 nm

### Example 77—Synthesis of N—((R)-1-cyano-3-(methylamino)-3-oxopropyl)-8-(4-(trifluoromethyl)cyclohex-1-en-1-yl)quinoline-3-carboxamide (Compound 83)

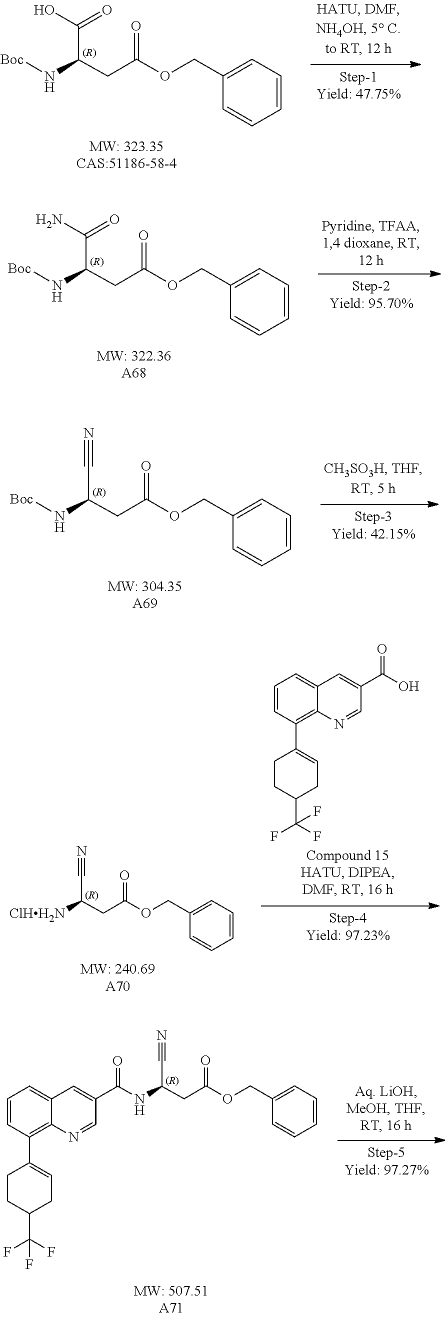

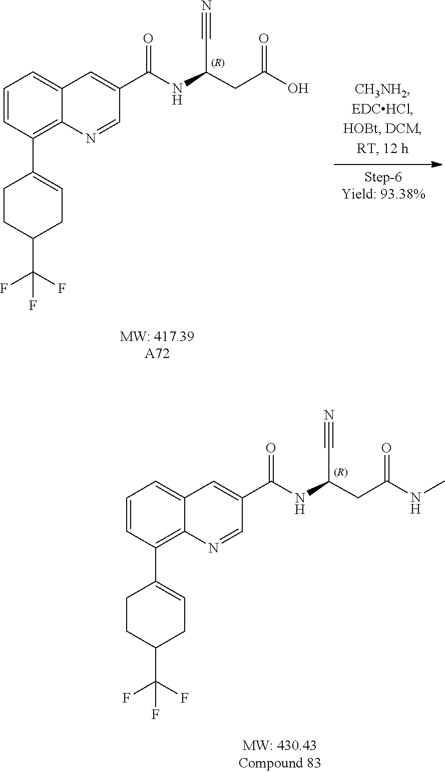

### Step 1

To a stirred solution of CAS: 51186-58-4 (0.5 g, 1.54 mmol, 1.0 eq.) in DMF (5.0 mL) was added HATU (0.88 g, 2.32 mmol, 1.5 eq.) at 0° C. and stirred for 1 h. followed by addition of aq. NH₄OH (0.37 mL 28%). The resulting reaction mixture was stirred at RT for 12 h. TLC indicated the completion of reaction, the resulting reaction mixture was diluted with water (10 mL) and extracted with EtOAc (3×20 mL). The combined organic layer was dried over anhydrous Na₂SO₄, filtered and concentrated under reduced pressure to obtain crude product. The crude product was purified by manual column chromatography (desired product eluted in 35% Ethyl acetate in hexane) to afford benzyl(R)-4-amino-3-((tert-butoxy carbonyl)amino)-4-oxobutanoate (A68) (0.238, 0.739 mmol, Yield 47.75%).

¹H NMR (MeOD, 400 MHz): δ ppm 7.37-7.30 (m, 5H), 7.27 (s, 1H), 7.10 (s, 1H), 7.05 (d, J=8.4 Hz, 1H), 5.12-5.05 (m, 2H), 4.31-4.26 (m, 1H), 2.78-2.73 (dd, J=5.6 Hz, 16.0 Hz, 1H), 2.61-2.47 (dd, J=5.2, 16.0 Hz, 1H), 1.33 (d, J=24.0 Hz, 9H).

LCMS (Method A): 1.821 min, 100.0% 210 nm, MS: ES+323.2 (M+1)

### Step-2

To a stirred solution of benzyl(R)-4-amino-3-((tert-butoxycarbonyl)amino)-4-oxobutanoate (A68) (4.0 g, 12.4 mmol, 1.0 eq.) in 1,4 Dioxane (40.0 mL) was added pyridine (4.0 mL, 13.6 mmol, 1.1 eq.) at RT and stirred for 10 min. After 10 min, TFAA (2.8 mL, 55.9 mmol, 4.5 eq.) was added dropwise and resulting reaction mixture was stirred at RT for 12 h. TLC indicated the completion of reaction, the resulting reaction mixture was diluted with NaHCO₃ (100 mL) and extracted with EtOAc (3×30 mL). The combined organic layer was dried over anhy Na₂SO₄, filtered and concentrated under reduced pressure to obtained crude material. The crude material was purified by manual column chromatography silica gel (100-200 mesh) (desired product eluted in 30% Ethyl acetate in hexane) to afford benzyl(R)-3-((tert-butoxycarbonyl)amino)-3-cyanopropanoate (A69) (3.6 g, 11.82 mol, Yield 95.70%).

¹H NMR (MeOD, 400 MHz): δ ppm 7.84 (d, J=7.6 Hz, 1H), 7.38-7.30 (m, 5H), 5.12 (s, 2H), 4.70 (d, J=6.8 Hz, 1H), 3.02-2.87 (m, 2H), 1.30 (s, 9H).

LCMS (Method A): 2.32 min, 90.42%, 220 nm, MS: ES+305.2 (M+1)

### Step-3

To a stirred solution of benzyl(R)-3-((tert-butoxy carbonyl)amino)-3-cyanopropanoate (A69) (3.6 g, 11.82 mmol, 1.0 eq.) in THF (30 mL) was added CH₃SO₃H (5.22 mL, 59.14 mmol, 5.0 eq.) at RT and resulting reaction mixture was stirred at room temperature for 5 h. TLC indicated the completion of reaction, the resulting reaction mixture was diluted with aq. NaHCO₃ (100 mL) and extracted with EtOAc (3×30 mL). The combined organic layer was dried over anhy. Na₂SO₄, filtered and concentrated under reduced pressure to afford crude product. The crude product was purified by manual column chromatography silica gel (100-200 mesh) (desired product eluted in 30% Ethyl acetate in hexane) to afford benzyl(R)-3-amino-3-cyanopropanoate hydrochloride (A70) (1.2 g, 4.98 mmol, Yield 42.15%).

¹H NMR (DMSO, 400 MHz): δ ppm 7.39-7.31 (m, 5H), 5.14 (s, 2H), 4.01 (t, J=6.8 Hz, 1H), 2.88-2.74 (m, 2H); LCMS (Method A): Not supported the desired Mass.

### Step-4

A stirred solution of benzyl(R)-3-amino-3-cyanopropanoate hydrochloride (A70) (0.5 g, 2.08 mmol, 1.0 eq.) in DMF (5.0 mL) was prepared. To this solution was added DIPEA (1.15 mL, 6.24 mmol, 3.0 eq.) and HATU (1.18 g, 3.12 mmol, 1.5 eq.) at RT under nitrogen atmosphere and stirred for 10 min. After 10 min, addition of 8-(4-(trifluoromethyl)cyclohex-1-en-1-yl)quinoline-3-carboxylic acid (Compound 15) (0.801 g, 2.28 mmol, 1.1 eq.) at same temperature and again resulting reaction mixture stirred at RT for 16 h. TLC indicated the completion of reaction, the resulting reaction mixture was diluted with water (30 mL) and extracted with EtOAc (3×20 mL). The combined organic layer was dried over anhy. Na₂SO₄, filtered, and concentrated under reduced pressure to afford crude product. The crude product was purified by manual column chromatography silica gel (100-200 mesh) (desired product eluted in 35% Ethyl acetate in hexane) to afford benzyl (3R)-3-cyano-3-(8-(4-(trifluoromethyl)cyclohex-1-en-1-yl)quinoline-3-carboxamido) propanoate (A71) (0.475, 0.936 mmol, Yield: 97.23%).

¹H NMR (DMSO, 400 MHz): δ ppm 9.60 (d, J=7.2 Hz, 1H), 9.22 (d, J=1.6 Hz, 1H), 8.81 (d, J=1.6 Hz, 1H), 8.04-8.02 (dd, J=2.4 Hz, & 6.8 Hz, 1H), 7.66 (t, J=7.2 Hz, 2H), 7.37 (d, J=6.8 Hz, 2H), 7.33-7.27 (m, 3H), 5.88 (br s, 1H), 5.32-5.27 (m, 1H), 5.18 (s, 2H), 3.34-3.15 (m, 2H), 2.81-2.78 (m, 1H), 2.70-2.65 (m, 2H), 2.46 (br s, 1H), 2.32-2.29 (m, 1H), 2.08-2.06 (m, 1H), 1.75-1.64 (m, 1H).

LCMS (Method A): 2.823 min, 100.0%, 254 nm, MS: ES+508.4 (M+1).

### Step-5

To a stirred solution of benzyl (3R)-3-cyano-3-(8-(4-(trifluoromethyl)cyclohex-1-en-1-yl)quinoline-3-carboxamido) propanoate (A71) (0.475 g, 0.931 mmol, 1.0 eq.) in MeOH:H₂O (8:2) was added LiOH·H₂O (0.064 g, 2.80 mmol, 3.0 eq.) and reaction mixture was stirred at RT for 16 h. TLC indicated the completion of reaction, the resulting reaction mixture was concentrated and residue was acidified by using 1N HCl up to pH acidic and extracted with EtOAc (3×20 mL). The combined organic layer was dried over anhy. Na₂SO₄, filtered, and concentrated under reduced pressure to afford (3R)-3-cyano-3-(8-(4-(trifluoromethyl)cyclohex-1-en-1-yl)quinoline-3-carboxamido) propanoic acid (A72) (0.380 g, 0.911 mmol, Yield 97.27%). Note: The crude product was directly used next step further without any purification.

LCMS (Method A): 2.359 min, 28.84%, 254.0 nm, ES+418.3 (M+1).

### Step-6

To a stirred solution of (3R)-3-cyano-3-(8-(4-(trifluoromethyl)cyclohex-1-en-1-yl)quinoline-3-carboxamido) propanoic acid (A72) (0.380 g, 0.911 mmol, 1.0 eq.) in DCM were added EDC·HCl (0.26 g, 1.36 mmol, 1.5 eq.) and HOBt (0.246 g, 1.82 mmol, 2.0 eq.) at RT and stirred for 15 min. Followed by addition of methylamine (0.9 MI, 1.82 mmol, 2.0 eq., 2M in THF) at RT and resulting reaction mixture stirred for 12 h. TLC indicated the completion of reaction, the resulting reaction mixture was diluted with water (10 Ml) and extracted with EtOAc (3×20 Ml). The combined organic layer was dried over anhy. Na₂SO₄ and concentrated under reduced pressure to afford crude product. The crude product was purified by manual column chromatography (desired product eluted in 35% Ethyl acetate in hexane) to afford N—((R)-1-cyano-3-(methylamino)-3-oxopropyl)-8-(4-(trifluoromethyl)cyclohex-1-en-1-yl)quinoline-3-carboxamide (Compound 83) (0.024 g, 0.056 mmol, Yield 47.75%)

¹H NMR (DMSO, 400 MHz): δ ppm 9.59 (d, J=7.2 Hz, 1H), 9.23 (d, J=2.4 Hz, 1H), 8.84 (d, J=2.4 Hz, 1H), 8.15 (d, J=4.8 Hz, 1H), 8.05-8.04 (dd, J=2.4, 6.8 Hz, 1H), 7.69-7.64 (m, 2H), 5.88 (br s, 1H), 5.26-5.20 (m, 1H), 2.92-2.90 (m, 1H), 2.85-2.80 (m, 2H), 2.69-2.67 (m, 2H), 2.62 (d, J=4.8 Hz, 3H), 2.33-2.25 (m, 2H), 2.08-2.04 (m, 1H), 1.73-1.67 (m, 1H).

HPLC (Method A): 7.98 min, 96.11%, 254 nm.

LCMS (Method A): 2.247 min, 100.0%, 254 nm, ES+431.3 (M+1).

Chiral HPLC: 5.37 min, 50%, 245 nm, 6.51 min, 49.8%, 245 nm

### Example 78—Synthesis of (R)—N-(1-cyano-3-(methylamino)-3-oxopropyl)-8-(4,4-difluorocyclohex-1-en-1-yl)quinoline-3-carboxamide (Compound 84)

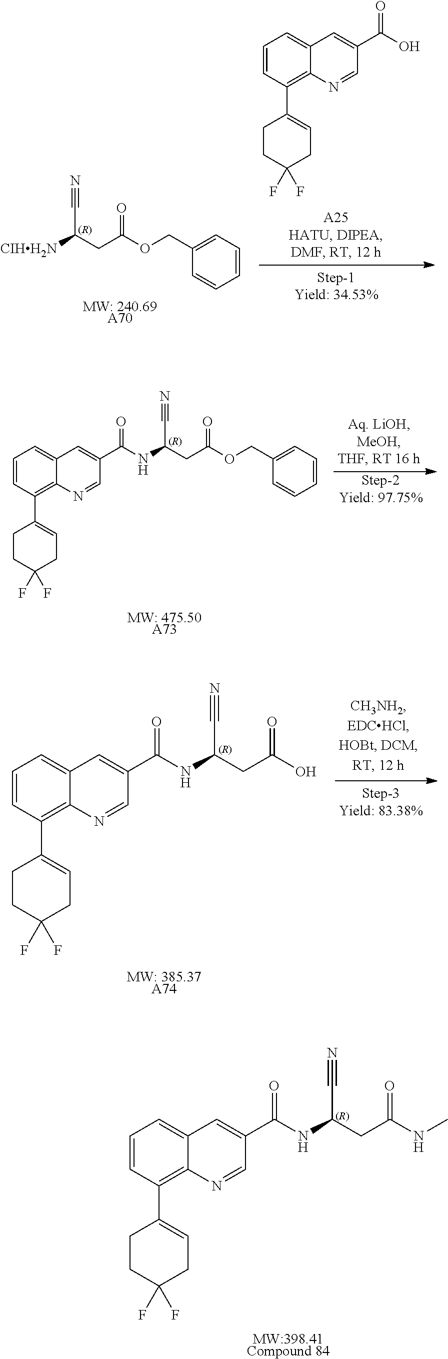

### Step-1

A stirred solution of A25 (0.70 g, 2.91 mmol, 1.2 eq), HATU (1.39 g, 3.60 mmol, 1.5 eq) and DIPEA (1.34 ml, 7.34 mmol, 3.0 eq) in DMF (5.0 mL) was prepared at RT under nitrogen atmosphere and stirred for 10 min. After 10 min, benzyl(R)-3-amino-3-cyanopropanoate hydrochloride (A70) (0.5 g, 2.43 mmol, 1.0 eq) was added and resulting reaction mixture was stirred at RT for 12 h. TLC indicated the completion of reaction, the resulting reaction mixture was diluted with Aq. NaHCO₃ (50 mL) and extracted with EtOAc (3×50 mL). The combined organic layer was dried over Na₂SO₄, filtered and concentrated under reduced pressure to afford 0.51 g crude product. The crude product was purified by manual column chromatography (desired product eluted in 35% Ethyl acetate in hexane) to afford benzyl(R)-3-cyano-3-(8-(4,4-difluorocyclohex-1-en-1-yl)quinoline-3-carboxamido) propanoate (A73) (0.4 g, 0.842 mmol, Yield 34.53%)

¹H NMR (DMSO, 400 MHz): δ ppm 9.60 (d, J=7.2 Hz, 1H), 9.23 (d, J=2.4 Hz, 1H), 8.82 (d, J=2.0 Hz, 1H), 8.05-8.04 (dd, J=1.6 Hz & J=7.6 Hz, 1H), 7.72-7.63 (m, 2H), 7.37 (d, J=6.8 Hz, 2H), 7.33-7.28 (m, 3H), 5.77 (s, 1H), 5.29 (m, 1H), 5.17 (s, 2H), 3.34-3.15 (m, 2H), 2.90 (br's, 2H), 2.79 (t, J=14.4 Hz, 2H), 2.28-2.18 (m, 2H).

LCMS (Method A): 2.616 min, 95.58%, 254 nm, MS: ES+476.34 (M+1).

### Step-2

To a stirred solution of a benzyl(R)-3-cyano-3-(8-(4,4-difluorocyclohex-1-en-1-yl)quinoline-3-carboxamido) propanoate (A73) (0.4 g, 0.842 mmol, 1.0 eq) in MeOH:H₂O:THF (7:2:1.10.0 mL) was added LiOH·H₂O (0.06 g, 2.52 mmol, 3.0 eq) and resulting reaction mixture was stirred for 16 h. TLC indicated the completion of reaction; the resulting reaction mixture was concentrated to afford crude material which was acidified by using IN HCl (50.0 mL) and extracted with EtOAc (3×20 mL). The combined organic layer was dried over anhy. Na₂SO₄, Filtered and concentrated under reduce pressure to afford (R)-3-cyano-3-(8-(4,4-difluorocyclohex-1-en-1-yl)quinoline-3-carboxamido) propanoic acid (A74) (0.38 g, 0.987 mmol, Yield 97.75%).

LCMS (Method A): 2.098 min, 71.57%, 254.0 nm, MS: ES+385.37 (M+1).

### Step-3

To a stirred solution of (R)-3-cyano-3-(8-(4,4-difluorocyclohex-1-en-1-yl)quinoline-3-carbox amido) propanoic acid (A74) (0.38 g, 0.911 mmol, 1.0 eq) in DCM (5.0 mL) was added EDC·HCl (0.26 g, 1.36 mmol, 1.5 eq) and HOBt (0.246 g, 1.82 mmol, 2.0 eq) at RT under nitrogen atmosphere. Then resulting reaction mixture stirred for 15 min. After 15 min, methylamine was added (0.9 mL, 1.82 mmol, 2.0 eq, 2M in THF) and again stirred for 12 h. TLC indicated the completion of reaction, the resulting reaction mixture was diluted with water (10 mL) and extracted with EtOAc (3×20 mL). The combined organic layer was dried over anhy. Na₂SO₄, filtered and concentrated under reduced pressure to afford 0.4 g crude product. The crude product was purified by manual column chromatography (desired product eluted in 35% Ethyl acetate in hexane) to afford (R)—N-(1-cyano-3-(methylamino)-3-oxopropyl)-8-(4,4-difluorocyclohex-1-en-1-yl)quinoline-3-carboxamide (Compound 84) (0.238 g, 0.739 mmol, Yield 47.75% 1H NMR (MeOD, 400 MHz): δ ppm 9.56 (d, J=7.2 Hz, 1H), 9.26 (d, J=2.0 Hz, 1H), 8.86 (d, J=2.0 Hz, 1H), 8.5 (d, J=4.0 Hz, 1H), 8.05 (t, J=6.4 Hz & 1.2 Hz, 1H), 7.71-7.65 (m, 2H), 5.77 (br's, 1H), 5.26-5.21 (m, 1H), 3.39 (s, 2H), 2.96-2.76 (m, 6H), 2.76 (s, 3H), 2.26-2.19 (m, 2H).

LCMS (Method A): 2.045 min, 100.0%, 244 nm, MS: ES+399.27 (M+1)

HPLC (Method A): 7.05 min, 96.39. %, 254 nm

### Example 79—Synthesis of N—((R)-4-amino-4-oxobutan-2-yl)-8-(4-(trifluoromethyl)cyclohex-1-en-1-yl)quinoline-3-carboxamide (Compound 85)

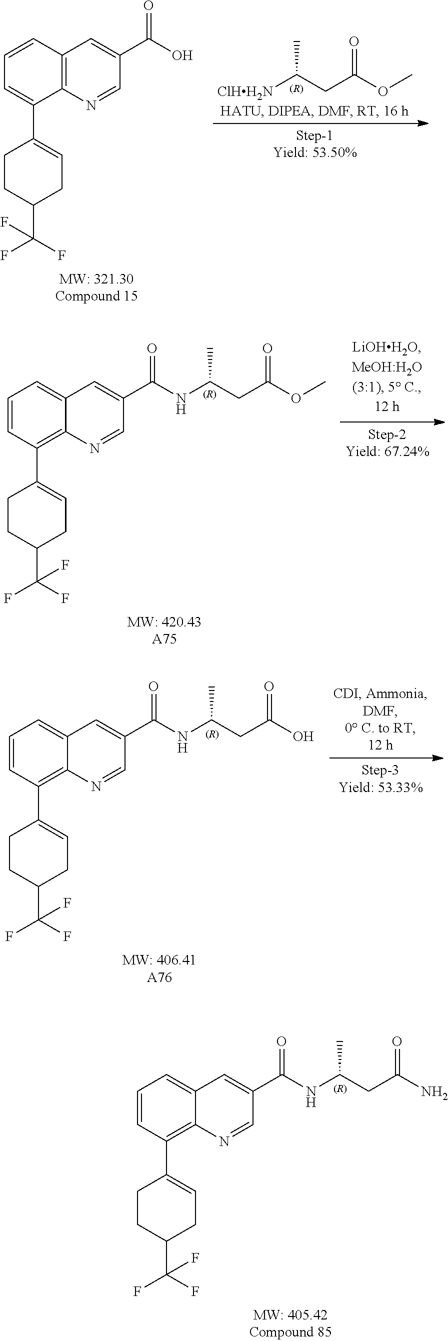

### Step-1

To a stirred solution of 8-(4-(trifluoromethyl)cyclohex-1-en-1-yl)quinoline-3-carboxylic acid (Compound 15) (0.3 g, 0.934 mmol, 1.0 eq.) in DMF (2.0 mL) were added DIPEA (0.542 g, 2.80 mmol, 3.0 eq.), and HATU (0.532 g, 1.40 mmol, 1.5 eq.) at RT under Nitrogen atmosphere and stirred for 15 min. After 15 min (R)-Methyl 3-aminobutanoate hydrochloride CAS: 139243-54-2 (0.157 g, 1.03 mmol, 1.1 eq.) was added and reaction mixture was stirred RT for 16 h. TLC indicated the completion of reaction, the resulting reaction mixture was diluted with water (10 mL) and extracted with EtOAc (3×20 mL). The combined organic layer was dried over anhy. Na₂SO₄, Filtered and concentrated under reduced pressure to afford crude product. The crude product was purified by manual column chromatography silica gel (100-200) (desired product eluted in 20% Ethyl acetate in hexane) to afford methyl (3R)-3-(8-(4-(trifluoromethyl)cyclohex-1-en-1-yl)quinoline-3-carboxamido) butanoate (A75) (0.210 g, 0.499 mmol, Yield: 53.45%).

¹H NMR (DMSO-d₆, 400 MHz): δ ppm 9.21 (d, J=2.0 Hz, 1H), 8.75 (d, J=2.0 Hz, 1H), 8.70 (d, J=7.6 Hz, 1H), 8.00 (dd, J=4.0 Hz & 9.6 Hz, 1H), 7.64-7.61 (m, 2H), 5.87 (br s, 1H), 4.45-4.38 (m, 1H), 3.60 (s, 3H), 2.79 (d, J=12.4 Hz, 1H), 2.72-2.66 (m, 3H), 2.57 (d, J=6.8 Hz, 2H), 2.32-2.24 (m, 1H), 2.08 (d, J=8.0 Hz, 1H), 1.71-1.67 (m, 1H), 1.24 (d, J=6.8 Hz, 3H).

LCMS (Method A): 2.487 min, 96.19%, 254 nm, MS: ES+421.2 (M+1)

### Step-2

To a stirred solution of methyl (3R)-3-(8-(4-(trifluoromethyl)cyclohex-1-en-1-yl)quinoline-3-carboxamido) butanoate (A75) (0.21 g, 0.499 mmol, 1.0 eq.) in MeOH:H₂O (3:1 mL), was added LiOH·H₂O (0.017 g, 0.749 mmol, 1.5 eq.) and resulting reaction mixture was stirred at room temperature. TLC indicated the completion of reaction; the resulting reaction mixture was concentrated followed by acidified using IN HCl solution up to pH 2-3 to obtain solid precipitate. Then precipitate was filtered on Buchner funnel and dried under reduce pressure to afford (3R)-3-(8-(4-(trifluoromethyl)cyclohex-1-en-1-yl)quinoline-3-carboxamido)butanoic acid (A76) (0.1 g, 0.246 mmol, Yield: 49.26%).

¹H NMR (MeOD, 400 MHz): δ ppm 9.29 (d, J=2.4 Hz, 1H), 9.05 (d, J=1.6 Hz, 1H), 8.10 (dd, J=1.6 Hz & 8.0 Hz, 1H), 7.85-7.77 (m, 2H), 5.95 (br s, 1H), 4.62-4.57 (m, 1H), 2.78-2.70 (m, 2H), 2.69-2.55 (m, 3H), 2.44-2.41 (m, 1H), 2.22-2.19 (m, 1H), 2.19-1.88 (m, 1H), 1.40 (d, J=4.0 Hz, 3H). Note: —CONH and —COOH proton exchanged in MeOD NMR.

HPLC (Method A): 4.431 min, 98.88%, 254 nm.

LCMS (Method A): 2.268 min, 98.02%, 242.0 nm, MS: ES+407.3 (M+1).

### Step-3

To a stirred solution of (3R)-3-(8-(4-(trifluoromethyl)cyclohex-1-en-1-yl)quinoline-3-carboxamido)butanoic acid (A76) (0.06 g, 0.147 mmol, 1.0 eq.) in DMF (2.0 mL) was added CDI (0.047 g, 0.295 mmol, 2.0 eq.) at 0° C. under nitrogen atmosphere. The resulting reaction mixture stirred at RT for 1 h. After 1 h, addition of Aq. Ammonia (1.0 mL, 28% NH₄OH) at 0° C. and again stirred at RT for 12 h. TLC indicated the completion of reaction, the resulting reaction mixture was diluted with water (10 mL) and extracted with EtOAc (3×20 mL). The combined organic layer was dried over anhy. Na₂SO₄, filtered and concentrated under reduced pressure to afford crude product. The crude product was purified by manual column chromatography (desired product eluted in 20% Ethyl acetate in hexane) to afford N—((R)-4-amino-4-oxobutan-2-yl)-8-(4-(trifluoromethyl)cyclohex-1-en-1-yl)quinoline-3-carboxamide (Compound 85) (0.032 g, 0.078 mmol, Yield: 53.33%).

¹H NMR (MeOD, 400 MHz): δ ppm 9.22 (d, J=2.4 Hz, 1H), 8.74 (d, J=2.0 Hz, 1H), 7.96 (dd, J=2.0 Hz & 7.6 Hz, 1H), 7.68-7.64 (m, 2H), 5.86 (br s, 1H), 4.58-4.57 (m, 1H), 2.82-2.78 (m, 1H), 2.68-2.61 (m, 3H), 2.53-2.48 (m, 2H), 2.44-2.41 (m, 1H), 2.22-2.19 (m, 1H), 2.19-1.88 (m, 1H), 1.38 (d, J=6.8 Hz, 3H). Note: —CONH and —CONH₂ proton exchanged in MeOD NMR.

HPLC (Method A): 7.436 min, 98.69%, 254 nm.

LCMS (Method A): 2.117 min, 100%, 210 nm, MS: ES+406.2 (M+1).

### Example 80—Synthesis of N—((R)-1-hydroxybutan-2-yl)-8-(4-(trifluoromethyl)cyclohex-1-en-1-yl)quinoline-3-carboxamide (Compound 86)

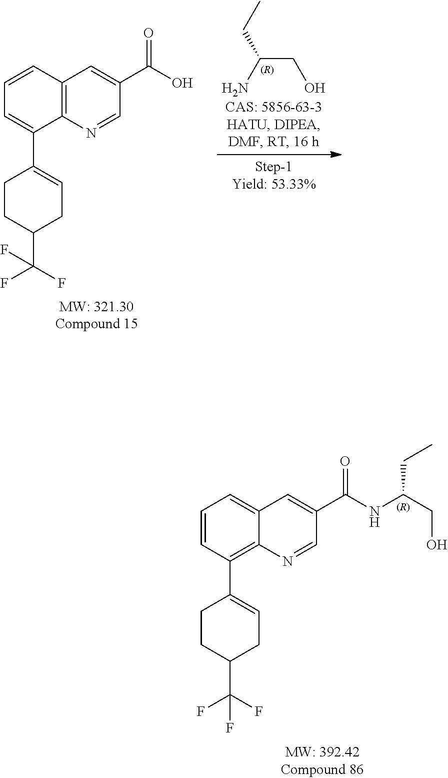

### Step-1

To a stirred solution of 8-(4-(trifluoromethyl)cyclohex-1-en-1-yl)quinoline-3-carboxylic acid Compound 15 (0.1 g, 0.31 mmol, 1.0 eq.) in DMF (2.0 mL) was added DIPEA (0.120 g, 0.93 mmol, 3.0 eq.) and HATU (0.177 g, 0.46 mmol, 1.5 eq.) at RT under nitrogen atmosphere and stirred for 15 min. followed by addition of CAS: 5856-63-6 (R)-(−)-2-Amino-1-butanol (0.030 g, 0.34 mmol, 1.1 eq.) and reaction mixture was stirred at RT for 16 h. TLC indicated the completion of reaction, the resulting reaction mixture was diluted with water (10 mL) and extracted with EtOAc (3×20 mL). The combined organic layer was dried over Na₂SO₄ and concentrated under reduced pressure to afford the crude product. The crude was further purified by manual column chromatography on silica gel (100-200) (desired product eluted in 100% Ethyl acetate) to afford N—((R)-1-hydroxybutan-2-yl)-8-(4-(trifluoromethyl)cyclohex-1-en-1-yl)quinoline-3-carboxamide (Compound 86) (0.064 g, 0.16 mmol, Yield: 53.33%)

¹H NMR (DMSO-d₆, 400 MHz): δ ppm 9.25 (d, J=1.6 Hz, 1H), 8.81 (d, J=1.6 Hz, 1H), 8.39 (d, J=8.4 Hz, 1H), 8.00 (t, J=4.8 Hz, 1H), 7.63 (d, J=4.4 Hz, 1H), 5.87 (br s, 1H), 4.74 (t, J=5.2 Hz, 1H), 3.93 (d, J=4.8 Hz, 1H), 3.52-3.46 (m, 2H), 2.77-2.67 (m, 3H), 2.32-2.28 (m, 1H), 2.08 (d, J=12.0 Hz, 1H), 1.73-1.67 (m, 2H), 1.50-1.48 (m, 1H), 0.91 (t, J=7.2 Hz, 3H). Note: CF₃—CH proton merged with DMSO solvent peak.

¹H NMR (MeOD, 400 MHz): δ ppm 9.26 (d, J=1.6 Hz, 1H), 8.79 (d, J=2.0 Hz, 1H), 7.97 (d, J=6.0 Hz, 1H), 7.69-7.63 (m, 2H), 5.87 (br s, 1H), 4.13-4.10 (m, 1H), 3.69 (d, J=4.4 Hz, 2H), 3.01-2.88 (m, 1H), 2.84-2.65 (m, 4H), 2.55-2.37 (m, 3H), 2.18-2.15 (m, 2H), 1.93-1.82 (m, 2H), 1.79-1.65 (m, 1H), 0.91 (t, J=7.2 Hz, 3H). Note: —CONH and —OH proton exchanged in MeOD NMR.

LCMS (Method-A): 2.30 min, 99.54%, 220 nm, MS: ES+393.3 (M+1)

HPLC (Method-A): 8.22 min, 95.85%, 210 nm

### Example 81—Synthesis of N—((R)-2-hydroxypropyl)-8-(4-(trifluoromethyl)cyclohex-1-en-1-yl)quinoline-3-carboxamide (Compound 87)

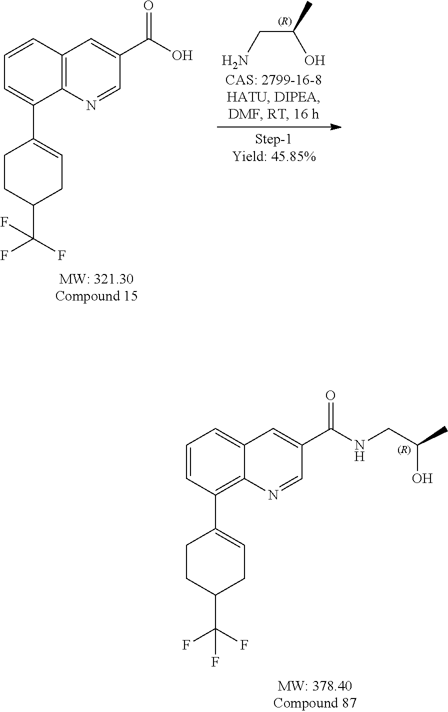

### Step-1

To a stirred solution of 8-(4-(trifluoromethyl)cyclohex-1-en-1-yl)quinoline-3-carboxylic acid Compound 15 (0.1 g, 0.311 mmol, 1.0 eq.) in DMF (1.0 mL) was added HATU (0.236 g, 0.623 mmol, 2.0 eq.), DIPEA (0.120 g, 0.934 mmol, 3.0 eq.) at 0° C. and stirred reaction mixture for 15 min. After 15 min, addition of CAS: 2799-16-8 (R)-(−)-1-Amino-2-propanol (0.025 g, 0.342 mmol, 1.1 eq.) and stirred reaction mixture at RT for 16 h. TLC indicated the completion of reaction, the resulting reaction mixture was diluted with water (10 mL) and extracted with EtOAc (3×20 mL). The combined organic layer was dried over anhy. Na₂SO₄, filtered, and concentrated under reduced pressure to afford crude product. The crude product was purified by manual column chromatography using silica gel (100-200) (desired product eluted in 40% Ethyl acetate in hexane) to afford N—((R)-2-hydroxypropyl)-8-(4-(trifluoromethyl)cyclohex-1-en-1-yl)quinoline-3-carboxamide (Compound 87) (0.054 g, 0.142 mmol, Yield: 45.85%).

¹H NMR (DMSO-d₆, 400 MHz): δ ppm, 9.25 (d, J=1.6 Hz, 1H), 8.82-8.75 (m, 2H), 8.00 (t, J=5.2 Hz, 1H), 7.64-7.61 (m, 2H), 5.86 (br s, 1H), 4.82 (d, J=4.4 Hz, 1H), 3.86-3.81 (m, 1H), 3.31-3.22 (m, 2H), 2.80-2.77 (m, 1H), 2.72-2.69 (m, 2H), 2.32-2.24 (m, 1H), 2.10-2.07 (m, 1H), 1.74-1.67 (m, 1H), 1.11 (d, J=6.0 Hz, 3H). Note: CF₃—CH proton merged with DMSO solvent peak.

¹H NMR (MeOD, 400 MHz): δ ppm, 9.26 (d, J=2.0 Hz, 1H), 8.78 (d, J=2.0 Hz, 1H), 7.97 (dd, J=7.6 Hz & 7.6 Hz, 1H), 7.68-7.62 (m, 2H), 5.86 (s, 1H), 4.08-4.00 (m, 1H), 3.57-3.49 (m, 1H), 3.43-3.37 (m, 1H), 2.78-2.75 (m, 1H), 2.68-2.64 (m, 2H), 2.55-2.50 (m, 1H), 2.40-2.33 (m, 1H), 2.17-2.15 (m, 1H), 1.92-1.82 (m, 1H), 1.26 (d, J=6.4 Hz, 3H). Note: —CONH and —OH proton exchanged in MeOD NMR.

LCMS (Method-A): 2.205 min, 99.21%, 220 nm, MS: ES+379.3 (M+1)

HPLC (Method-A): 7.83 min, 97.48%, 254 nm

Chiral HPLC: 2.98 min, 49.5%, 240 nm; 3.44 min, 49.5%, 240 nm

### Example 82—Synthesis of N—((S)-2-hydroxypropyl)-8-(4-(trifluoromethyl)cyclohex-1-en-1-yl)quinoline-3-carboxamide (Compound 88)

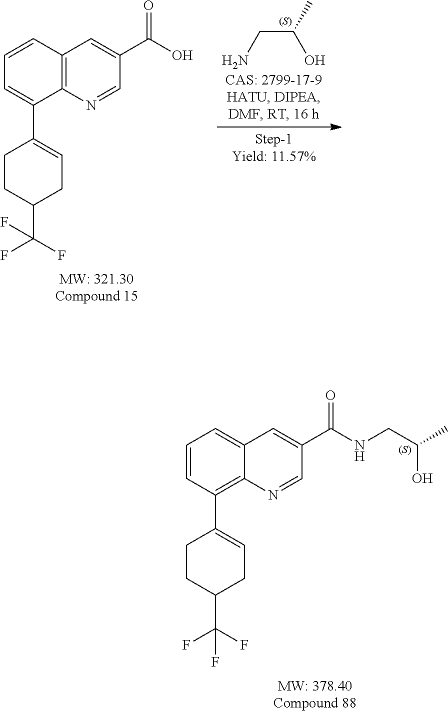

### Step-1

To a stirred solution of 8-(4-(trifluoromethyl)cyclohex-1-en-1-yl)quinoline-3-carboxylic acid (Compound 15) (0.1 g, 0.311 mmol, 1.0 eq.) in DMF (1 mL) were added HATU (0.177 g, 0.468 mmol, 1.5 eq.) and DIPEA (0.15 mL, 0.936 mmol, 3.0 eq.) at RT under nitrogen atmosphere, followed by cooling at 0° C. then(S)-1-aminopropan-2-ol CAS No: 2799-17-9 (0.026 g, 0.342 mmol, 1.1 eq.) was added into the reaction at 0° C. and stirred at RT for 16 h. TLC indicated the completion of reaction, the resulting reaction mixture was diluted with cold water (30 mL) and extracted with EtOAc (30 mL×3) combined organic layer were dried over Na₂SO₄ and evaporated under vacuum to get crude product. The crude was purified by column chromatography using silica gel (230-400 mesh) (eluting with 45-50% Ethyl Acetate in Hexane) to afford N—((S)-2-hydroxypropyl)-8-(4-(trifluoromethyl)cyclohex-1-en-1-yl)quinoline-3-carboxamide (Compound 88) (0.014 g, 0.037 mmol, Yield: 11.57%).

¹H NMR (DMSO-d₆, 400 MHz): δ ppm 9.26 (d, J=2.0 Hz, 1H), 8.82 (d, J=2.0 Hz, 1H), 8.77 (t, J=5.2 Hz, 1H), 8.00 (q, J=4 Hz, 1H), 7.64-7.61 (m, 2H), 5.87 (br s, 1H), 4.82 (d, J=4.4 Hz, 1H), 3.86-3.81 (m, 1H), 3.29-3.23 (m, 2H), 2.80-2.67 (br s, 2H), 2.30-2.25 (br s, 1H), 2.08 (d, J=8.8 Hz, 1H), 1.74-1.64 (m, 2H), 1.11 (d, J=6.4 Hz, 2H). Note: CF3-CH proton merged with DMSO solvent peak.

¹H NMR (MeOD, 400 MHz): δ ppm 9.26 (d, J=2.0 Hz, 1H), 8.79 (d, J=2.0 Hz, 1H), 7.97 (dd, J=1.6 Hz, J=7.6 Hz, 1H), 7.69-7.63 (m, 2H), 5.87 (br s, 1H), 4.04 (q, J=6.4 Hz, 1H), 3.54-3.49 (m, 1H), 3.41 (t, J=6.4 Hz, 2H), 2.78-2.75 (m, 1H), 2.66 (d, J=13.6 Hz, 2H), 2.54 (d, J=17.6 Hz 1H), 2.38 (d, J=13.6 Hz, 1H) 2.16 (d, J=10 Hz, 1H), 1.90-1.85 (m, 1H), 1.30 (s, 1H), 1.26 (d, J=6.4 Hz, 3H). Note: —CONH and —OH proton exchanged in MeOD NMR

HPLC (Method-A): 7.84 min, 96.09%, 254 nm

LCMS (Method-A): 2.198 min, 97.68%, 254.0 nm, MS: ES+379.27 (M+1) Chiral HPLC: 6.3 min, 47.6%, 254 nm; 6.5 min, 47.5%, 254 nm

### Example 83—Synthesis of N-((2R)-1-(((8-(4-(trifluoromethyl)cyclohex-1-en-1-yl) quinolin-3-yl)methyl)amino) propan-2-yl) acetamide and N—((R)-2-aminopropyl)-8-(4-(trifluoromethyl)cyclohex-1-en-1-yl)quinoline-3-carboxamide (Compound 89 and Compound 90)

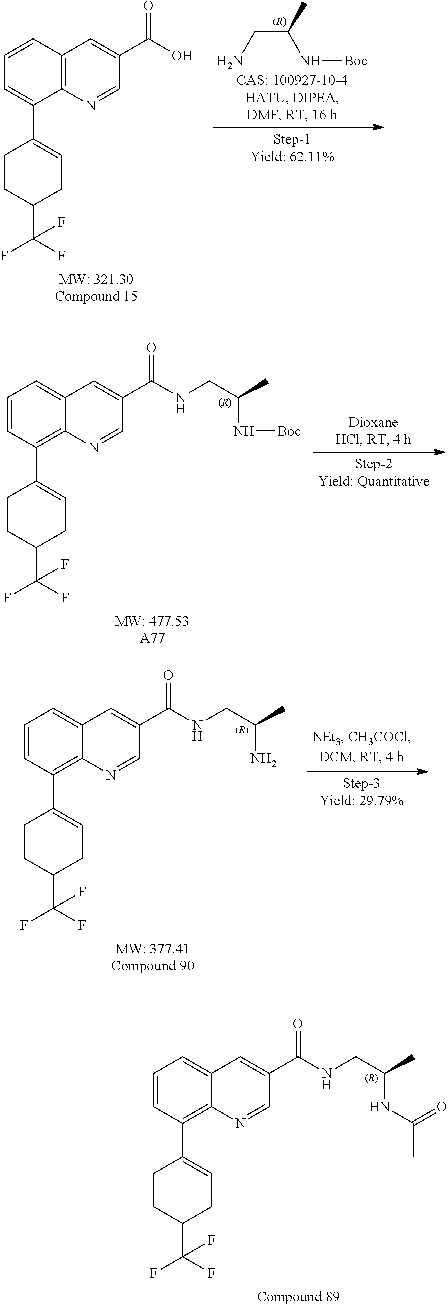

### Step-1

To a stirred solution of 8-(4-(trifluoromethyl)cyclohex-1-en-1-yl)quinoline-3-carboxylic acid (Compound 15) (0.1 g, 0.311 mmol, 1.0 eq.) in DMF (2 mL) were added HATU (0.177 g, 0.467 mmol, 1.5 eq.) and DIPEA (0.16 mL, 0.934 mmol, 3.0 eq.) under nitrogen atmosphere, followed by addition of CAS: 100927-10-4 (R)-tert-Butyl (1-aminopropan-2-yl) carbamate (0.065 g, 0.373 mmol, 1.2 eq.) and then reaction mixture was stirred at RT for 16 h TLC indicated the completion of reaction, the resulting reaction mixture was diluted with cold water (30 mL) and extracted with DCM (30 mL×3), combined organic layer were dried over Na₂SO₃ and evaporate under vacuum to get crude product. The crude was purified by column chromatography using silica gel (230-400 mesh) (eluting with 70% Ethyl Acetate in Hexane) to afford tert-butyl ((2R)-1-(8-(4-(trifluoromethyl)cyclohex-1-en-1-yl)quinoline-3-carbox amido) propan-2-yl) carbamate (A77) (0.12 g, 0.251 mmol, Yield: 62.11%).

¹H NMR (DMSO-d₆, 400 MHz): 9.24 (s, 1H), 8.78 (br s, 2H), 7.99 (t, J=4.4 Hz, 1H), 7.65-7.61 (m, 2H), 6.81 (d, J=8.4 Hz, 1H), 5.87 (br s, 1H), 3.79-3.76 (m, 1H), 3.41-3.28 (m, 2H), 2.89-2.68 (m, 4H), 2.33-2.25 (m, 1H), 2.10-2.07 (m, 1H), 1.73-1.67 (m, 1H), 1.35 (s, 9H), 1.07 (d, J=6.8 Hz, 3H).

LCMS (Method-A): 2.683 min, 99.16%, 242.0 nm, MS: ES+478.4 (M+1).

### Step-2

To a solution tert-butyl ((2R)-1-(8-(4-(trifluoromethyl)cyclohex-1-en-1-yl)quinoline-3-carboxamido) propan-2-yl) carbamate (A77) (0.12 g, 0.251 mmol, 1.0 eq.) in MeOH (4 mL) was added 4M HCl in Dioxane (0.13 mL, 0.502 mmol, 2.0 eq.) and stirred the reaction mixture at RT for 4 h. TLC indicated the completion of reaction, the reaction mixture was concentrated under reduced pressure to afford crude. The obtained crude material was purified by trituration with pentane to afford N—((R)-2-aminopropyl)-8-(4-(trifluoromethyl)cyclohex-1-en-1-yl)quinoline-3-carboxamide (Compound 90) (0.11 g, 0.291 mmol, quantitative).

¹H NMR (DMSO-d₆, 400 MHz): δ ppm, 9.34 (d, J=1.6 Hz, 1H), 9.19 (t, J=5.6 Hz, 1H), 8.96 (s, 1H), 8.1 (brs, 3H), 8.05-8.02 (m, 1H), 7.68-7.65 (m, 2H), 5.88 (br s, 1H), 3.59-3.45 (m, 3H), 2.80-2.68 (m, 3H), 2.41-2.25 (m, 1H), 2.10-2.07 (m, 1H), 1.76-1.67 (m, 1H), 1.26 (d, J=6.4 Hz, 3H).

¹H NMR (MeOD, 400 MHz): δ ppm, 9.57 (s, 1H), 9.49 (s, 1H), 8.34 (d, J=8.0 Hz, 1H), 8.07 (d, J=6.4 Hz, 1H), 8.00 (d, J=8.0 Hz, 1H), 6.07 (br s, 1H), 3.75-3.72 (m, 2H), 3.68-3.65 (m, 1H), 2.75-2.62 (m, 4H), 2.49-2.46 (m, 1H), 2.27-2.24 (m, 1H), 1.98-1.93 (m, 1H), 1.43 (d, J=6.4 Hz, 3H). Note: —CONH and —NH₂ proton Exchanged in MeOD NMR.

LCMS (Method A): 1.882 min, 98.14%, 254.0 nm, MS: ES+378.3 (M+1)

HPLC (Method A): 9.61 min, 95.28%, 254.0 nm

### Step-3

To the solution of N—((R)-2-aminopropyl)-8-(4-(trifluoromethyl)cyclohex-1-en-1-yl)quinoline-3-carboxamide (Compound 90) (0.05 g, 0.132 mmol, 1.0 eq.) in DCM (2 mL) was added TEA (0.55 mL, 0.397 mmol, 3.0 eq.) at 0° C. under nitrogen atmosphere and stirred for 10 min, then CH₃COCl (0.009 mL, 0.132 mmol, 1.0 eq.) was added and the reaction mixture continued stirred for 4 h at room temperature. TLC indicated the completion of reaction; the resulting reaction mixture was quenched with ice cold water (5 mL) and extracted with DCM (10 mL) The combined organic layers were dried over Na₂SO₄ and concentrated under reduced pressure to afford crude. The obtained crude material was purified by column chromatography using silica gel (230-400 mesh) (gradient elution 5% MeOH in DCM) to afford N-((2R)-1-((8-(4-(trifluoromethyl)cyclohex-1-en-1-yl) quinolin-3-yl)methyl)amino)propan-2-yl) acetamide (Compound 89) (0.016 g, 0.038 mmol, Yield: 29.79%).

¹H NMR (DMSO-d₆, 400 MHz): δ ppm, 9.23 (d, J=1.6 Hz, 1H), 8.80-8.78 (m, 2H), 8.00 (t, J=5.2 Hz, 1H), 7.82 (d, J=8.0 Hz, 1H), 7.65-7.64 (m, 2H), 5.87 (br s, 1H), 4.05-4.01 (m, 1H), 2.81-2.68 (m, 4H), 2.35-2.29 (m, 1H), 2.10-2.07 (m, 1H), 1.81 (s, 3H), 1.75-1.66 (m, 1H), 1.08 (d, J=6.4 Hz, 3H). Note: CF₃—CH proton merged with DMSO solvent peak.

¹H NMR (MeOD, 400 MHz): δ ppm, 9.24 (d, J=2.0 Hz, 1H), 8.73 (d, J=2.0 Hz, 1H), 7.99-7.97 (m, 1H), 7.69-7.63 (m, 2H), 5.87 (br s, 1H), 4.26-4.23 (m, 1H), 3.59-3.45 (m, 3H), 2.78-2.65 (m, 3H), 2.56-2.51 (m, 1H), 2.41-2.37 (m, 1H), 2.18-2.16 (m, 1H), 1.96 (s, 3H), 1.24 (d, J=6.8 Hz, 3H).

LCMS (Method A): 2.192 min, 99.24%, 242.0 nm, MS: ES+420.4 (M+1)

HPLC (Method A): 7.72 min, 97.80%, 254.0 nm

### Example 84—Synthesis of N-(2-(methylsulfonamido)ethyl)-8-(4-(trifluoromethyl)cyclohex-1-en-1-yl)quinoline-3-carboxamide (Compound 91)

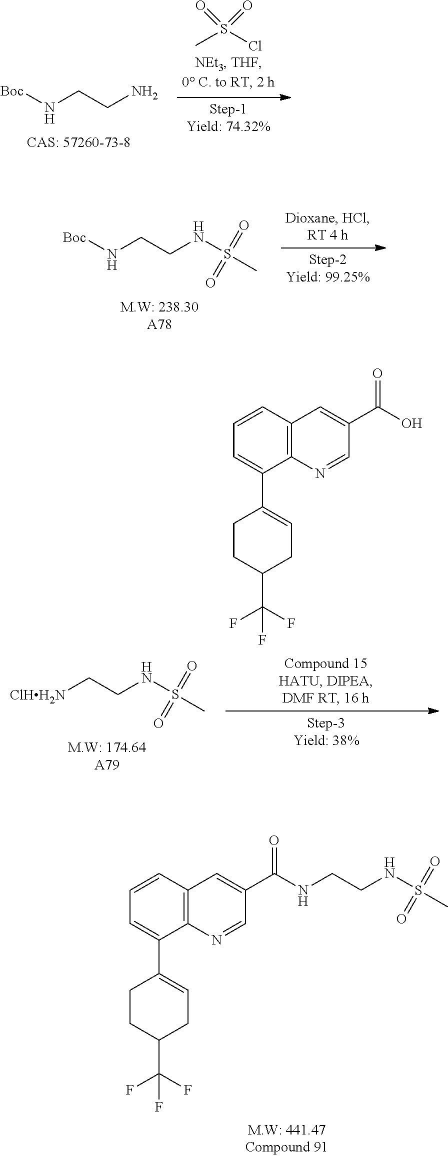

### Step-1

A stirred solution of tert-butyl (2-aminoethyl) carbamate CAS: 57260-73-8 (1.0 g, 6.24 mmol, 1.0 eq) in THF (10 mL) was prepared in 30 mL glass vial at room temperature. To this reaction solution, NEt₃ (0.5 g, 4.99 mmol, 0.8 eq) was added at room temperature. Followed by addition of methyl sulphonyl chloride (1.082 g, 7.48 mmol, 1.2 eq) at 0° C. The reaction mixture was stirred at RT for 2 h. TLC indicated the completion of reaction, the resulting reaction mixture was diluted with water (30 mL) and extracted with EtOAc (30 mL) three times, organic layer was dried over Na₂SO₃ and evaporated under vacuum to afford crude tert-butyl (2-(methylsulfonamido)ethyl) carbamate (A78) (1.1 g, 4.61 mmol, Yield: 74.32%).

¹H NMR (DMSO-d₆, 400 MHz): δ ppm 7.04 (bs, 1H), 6.88 (bs, 1H), 3.01 (t, J=6.0 Hz, 2H), 2.95 (t, J=5.6 Hz, 2H), 2.88 (s, 3H), 1.37 (s, 9H).

LCMS (Method-A): not supported

### Step-2

A stirred solution of tert-butyl (2-(methylsulfonamido)ethyl) carbamate (A78) (1.1 g, 4.61 mmol, 1.0 eq) in DCM (10.0 mL) was prepared in 30 mL glass vial at room temperature. To this reaction solution, 4M HCl in dioxane (6.89 mL, 13.84 mmol, 3.0 eq) was added at 0° C. and stirred the reaction mixture at RT for 2 h. TLC indicated the completion of reaction, the resulting reaction mixture was distilled under vacuum to afford crude N-(2-aminoethyl) methanesulfonamide hydrochloride (A79) (0.8 g, 4.58 mmol, Yield: 85.99%).

¹H NMR (DMSO-d₆, 400 MHz): δ ppm 8.16 (bs, 3H) 7.39 (t, J=6 Hz, 1H), 3.21 (q, J=6.4 Hz, J=12.8 Hz, 2H), 2.96 (s, 3H) 2.90-2.88 (m, 2H), 1.37 (bs, 1H),

LCMS (Method-A): not supported

### Step-3

To a stirred solution of N-(2-aminoethyl) methane sulfonamide hydrochloride (A79), Compound 15 (0.1 g, 0.312 mmol, 1.0 eq.) in DMF in 10 mL glass vial was added HATU (0.216, 0.57 mmol, 1.5 eq), DIPEA (0.12 g, 0.936 mmol, 3.0 eq.) then reaction mixture was cooled up to 0° C. The N-(2-aminoethyl) methane sulfonamide hydrochloride (A79) (0.065 g, 0.374 mmol, 1.2 equiv) was added and the resulting reaction mixture was stirred at RT for 16 h. TLC indicated the completion of reaction, the resulting reaction mixture was diluted with cold water (30 mL) and the fallout precipitate was filtered and dried under vacuum. Crude obtained was purified by trituration with pentane to afford N-(2-(methylsulfonamido)ethyl)-8-(4-(trifluoromethyl)cyclohex-1-en-1-yl)quinoline-3-carboxamide (Compound 91) (0.053 g, 0.120 mmol, Yield: 38%)

¹H NMR (DMSO-d₆, 400 MHz): δ ppm 9.26 (d, J=2 Hz, 1H), 8.90 (t, J=5.6 Hz, 1H), 8.81 (d, J=1.6 Hz, 1H), 8.02-8.01 (m, 1H), 7.65-7.62 (m, 2H), 7.22 (t, J=5.6 Hz, 1H), 5.87 (bs, 1H), 3.45-3.43 (t, J=6.0 Hz, 2H) 3.23-3.10 (m, 2H), 2.93 (s, 3H), 2.80-2.77 (m, 1H), 2.72-2.67 (m, 2H), 2.39-2.25 (m, 1H), 2.10-2.07 (m, 1H), 1.74-1.64 (m, 1H)

¹H NMR (MeOD, 400 MHz): δ ppm 9.26 (d, J=2 Hz, 1H), 8.79 (d, J=2.0 Hz, 1H), 7.97 (dd, J=1.6 Hz, J=7.6 1H), 7.69-7.63 (m, 2H), 5.87 (bs, 1H), 3.61 (t, J=6.0 Hz, 2H), 2.99 (bs, 3H), 2.81-2.75 (m, 1H), 2.68-2.65 (m, 2H), 2.55-2.51 (m, 1H), 2.40-2.34 (m, 2H), 2.18-2.15 (m, 1H), 1.93-1.85 (m, 1H).

LCMS (Method A): 2.260 min, 97.85%, MS: ES+442.88 [M+H]

HPLC (Method B): 6.88 min, 95.71%, 254 nm

### Example 85—Synthesis of methyl (2-(8-(4-(trifluoromethyl)cyclohex-1-en-1-yl)quinoline-3-carboxamido)ethyl) carbamate (Compound 92)

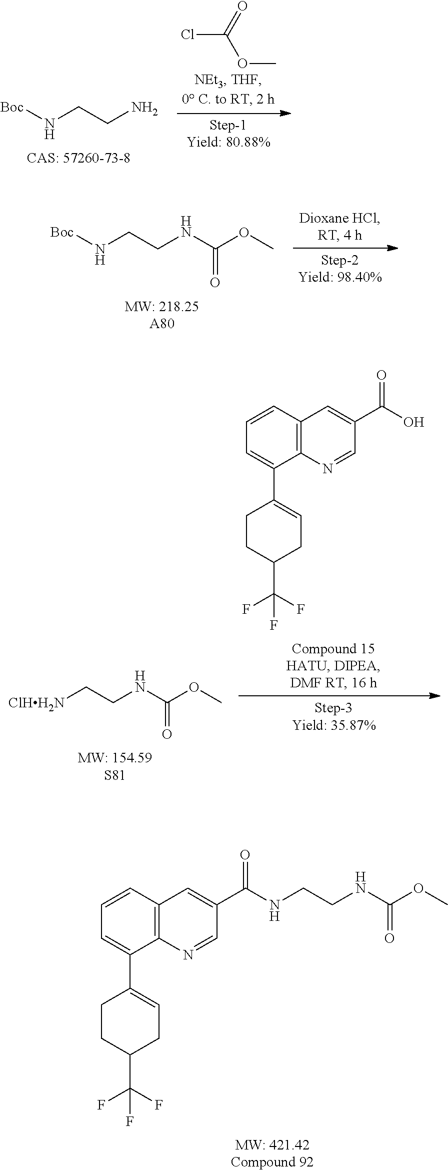

### Step-1

A stirred solution of tert-butyl (2-aminoethyl) carbamate CAS: 57260-73-8 (0.1 g, 0.624 mmol, 1.0 eq.) in THF (10 mL) was prepared in 30 mL glass vial at room temperature. To this reaction solution, NEt₃ (0.05 g, 0.499 mmol, 0.8 eq.) was added followed by addition of methyl chloroformate (0.088 g, 0.936 mmol, 1.5 eq.) at 0° C. Then the reaction mixture was stirred at RT for 2 h. TLC indicated the completion of reaction, the resulting reaction mixture was diluted with water (30 mL), extracted with EtOAc (30 mL) three times, organic layer was dried over Na₂SO₄ and evaporated under vacuum to afford to pure tert-butyl methyl ethane-1,2-diyldicarbamate (A80) (0.11 g, 0.504 mmol, Yield: 80.88%).

¹H NMR (DMSO-d₆, 400 MHz): δ ppm 7.11 (br s, 1H), 6.82 (br s, 1H), 3.50 (s, 3H), 2.96 (m, 4H), 1.37 (s, 9H); LCMS (Method A): not supported

### Step-2

A stirred solution of tert-butyl methyl ethane-1,2-diyldicarbamate (A80) (0.11 g, 0.504 mmol, 1.0 eq.) in DCM (10 mL) were prepared in 10 mL glass vial at room temperature. To this reaction solution 4M HCl in dioxane (0.88 mL, 1.50 mmol, 3.0 eq.) was added at 0° C. and stirred the reaction mixture at RT for 2 h. TLC indicated the completion of reaction, the resulting reaction mixture was distilled under vacuum to afford pure methyl (2-aminoethyl) carbamate hydrochloride (A81) (0.096 g, 0.620 mmol, Yield: 98.40%).

¹H NMR (DMSO-d₆, 400 MHz): δ ppm 7.97 (br s, 3H) 7.33 (br s, 1H), 3.54 (s, 3H), 3.38 (s, 3H) 3.23 (q, J=6 Hz, 2H), 2.84 (q, J=6 Hz, 2H) Note: Proton count is more due to HCl Salt.

LCMS (Method A): not supported

### Step-3

To the stirred solution of 8-(4-(trifluoromethyl)cyclohex-1-en-1-yl)quinoline-3-carboxylic acid (Compound 15) (0.1 g, 0.311 mmol, 1.0 eq.) in DMF was added DIPEA (0.15 mL, 0.924 mmol, 3.0 eq.) followed by addition of methyl (2-aminoethyl) carbamate hydrochloride (A81) (0.057 g, 0.373 mmol, 1.2 equiv) and resulting reaction mixture was stirred at RT for 16 h. TLC indicated the completion of reaction, the resulting reaction mixture was diluted with cold water (30 mL) and the fallout precipitate filtered and dried under vacuum then crude was purified by trituration with pentane to afford methyl (2-(8-(4-(trifluoromethyl)cyclohex-1-en-1-yl)quinoline-3-carboxamido)ethyl) carbamate (Compound 92) (0.047 g, 0.111 mmol, Yield: 35.87%)

¹H NMR (DMSO-d₆, 400 MHz): δ ppm 9.24 (d, J=2.0 Hz, 1H), 8.86 (t, J=5.2 Hz, 1H), 8.79 (d, J=2 Hz, 1H), 8.01-7.99 (m, 1H), 7.64-7.61 (m, 2H), 7.31 (t, J=5.6 Hz, 1H), 5.87 (br s, 1H), 3.53 (s, 3H) 3.22-3.18 (m, 2H), 2.80-2.61 (m, 3H), 2.21 (br s, 1H), 2.09-2.06 (m, 1H), 1.74-1.64 (m, 1H), 1.25-1.23 (m, 1H). Note: CF₃—CH proton merged with DMSO solvent peak. 1H NMR (MeOD, 400 MHz): δ ppm 9.24 (d, J=2.4 Hz, 1H), 8.75 (t, J=2.4 Hz, 1H), 7.97 (dd, J=1.6 Hz, J=7.6 1H), 7.69-7.63 (m, 2H), 5.86 (br s, 1H), 3.65 (s, 3H), 3.57 (t, J=6 Hz, 2H), 3.41 (t, J=6.4 Hz, 3H), 2.78-2.75 (m, 1H), 2.68-2.65 (m, 2H), 2.55-2.51 (m, 1H), 2.4-2.34 (m, 1H), 2.16 (d, J=10.8 Hz, 1H), 1.93-1.83 (m, 1H). Note: —CONH proton Exchanged in MeOD NMR.

LCMS (Method A): 2.315 min, 98.12%, 241.0 nm MS: ES+422.53 [M+H]

HPLC (Method A): 8.12 min, 95.61%, 254.0 nm

### Example 86—Synthesis of N-(3-(methylsulfonamido)propyl)-8-(4-(trifluoromethyl)cyclohex-1-en-1-yl)quinoline-3-carboxamide (Compound 93)

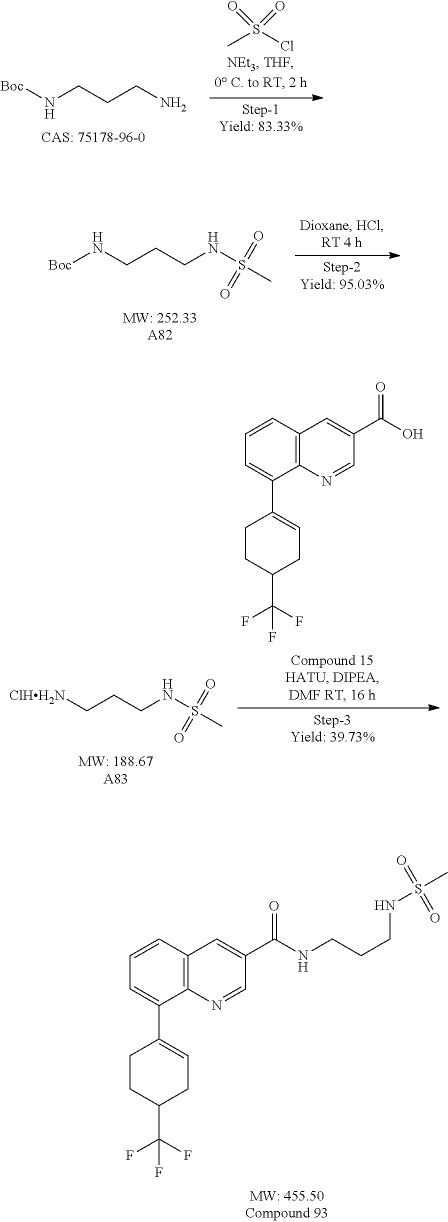

### Step-1

A stirred solution of tert-butyl (3-aminopropyl)carbamate CAS: 75178-96-0 (0.5 g, 2.869 mmol, 1.0 eq.) in THF (10 mL) was prepared at room temperature. To this reaction solution, NEt₃ (0.231 g, 2.295 mmol, 0.8 eq.) was added followed by addition of methyl sulfonyl chloride (0.39 g, 3.443 mmol, 1.2 eq.) at 0° C. and reaction mixture was stirred at RT for 2 h. TLC indicated the completion of reaction, the resulting reaction mixture was diluted with water (30 mL) and extracted with EtOAc (30 mL) three times, organic layer was dried over Na₂SO₄ and evaporated under vacuum to afford tert-butyl (3-(methylsulfonamido)propyl)carbamate (A82) (0.8 g, 3.174 mmol, Quantitative).

¹H NMR (DMSO-d₆, 400 MHz): δ ppm 6.93 (br s, 1H), 6.82 (br s, 1H), 4.18 (br s, 3H), 2.95-2.90 (m, 4H), 2.87 (s, 3H), 2.37 (s, 1H), 1.59-1.54 (m, 3H), 1.37 (s, 9H). Note: Exchangeable proton count is more.

LCMS (Method A): not supported

### Step-2

A stirred solution of tert-butyl (3-(methylsulfonamido)propyl)carbamate (A82) (0.8 g, 3.173 mmol, 1.0 eq.) in DCM (8 mL) were prepared in 30 mL glass vial at room temperature. To this reaction solution 4M HCl in dioxane (2.38 mL, 9.51 mmol, 3.0 eq.) was added at 0° C. and stirred the reaction mixture at RT for 2 h. TLC indicated the completion of reaction, the resulting reaction mixture was distilled under vacuum to afford N-(3-aminopropyl) methane sulfonamide hydrochloride (A83) (0.7 g, 3.71 mmol, Yield: 95.03%).

¹H NMR (DMSO-d₆, 400 MHz): δ ppm 8.032 (br s, 3H) 7.17 (t, J=6 Hz, 1H), 3.03 (q, J=6.4 Hz, 2H), 2.90 (s, 3H) 2.85-2.77 (m, 2H) 1.79-1.74 (m, 2H).

LCMS (Method A): not supported

### Step-3

To a stirred solution of 8-(4-(trifluoromethyl)cyclohex-1-en-1-yl)quinoline-3-carboxylic acid (Compound 15) (0.1 g, 0.311 mmol, 1.0 eq.) in DMF was added DIPEA (0.15 mL, 0.933 mmol, 3.0 eq.) and HATU (0.17 g, 0.466 mmol, 1.5 eq.) at RT. Followed by addition of N-(3-aminopropyl) methanesulfonamide hydrochloride (A83) (0.070 g, 0.373 mmol, 1.2 eq.) and the resulting reaction mixture was stirred at RT for 16 h. TLC indicated the completion of reaction, the resulting reaction mixture was diluted with cold water (30 mL) and the fallout precipitate was filtered then dried under vacuum. Crude obtained was purified by trituration with pentane to afford N-(3-(methylsulfonamido)propyl)-8-(4-(trifluoromethyl)cyclohex-1-en-1-yl)quinoline-3-carboxamide (Compound 93) (0.091 g, 0.199 mmol, Yield: 39.75%)

¹H NMR (DMSO-d₆, 400 MHz): δ ppm 9.23 (d, J=2.4 Hz, 1H), 8.82-8.79 (m, 2H), 8.022-7.99 (m, 1H), 7.64-7.61 (m, 2H), 7.04 (t, J=5.6 Hz, 1H), 5.86 (br s, 1H), 3.05 (q, J=6.8 Hz, 2H), 2.91 (s, 3H), 2.80-2.77 (d, 1H), 2.72-2.67 (br s, 2H), 2.32-2.24 (m, 1H), 2.10-2.07 (m, 1H), 1.81-1.73 (m, 2H), 1.71-1.64 (m, 1H). Note: —CH₂— and —CF₃CH proton merged in DMSO solvent peaks.

¹H NMR (MeOD, 400 MHz): δ ppm 9.25 (d, J=2.4 Hz, 1H), 8.78 (d, J=2.0 Hz, 1H), 7.97 (dd, J=4 Hz, J=8 Hz, 1H), 7.69-7.63 (m, 2H), 5.86 (br s, 1H), 3.57 (t, J=6.8 Hz, 2H), 3.21 (t, J=6.8 Hz, 2H), 2.96 (s, 3H), 2.80 (d, 1H), 2.80-2.75 (m, 1H), 2.66-2.63 (m, 1H), 2.55-2.51 (m, 1H), 2.18-2.15 (m, 1H), 1.96-1.89 (m, 2H). Note: —CONH and —NHSO₂Me protons are exchanged.

LCMS (Method A): 2.297 min, 100%, 210 nm MS: ES+456.48 [M+H]

HPLC (Method A): 8.03 min, 96.21%, 254 nm

### Example 87—Synthesis of methyl (3-(8-(4-(trifluoromethyl)cyclohex-1-en-1-yl)quinoline-3-carboxamido)propyl)carbamate (Compound 94)

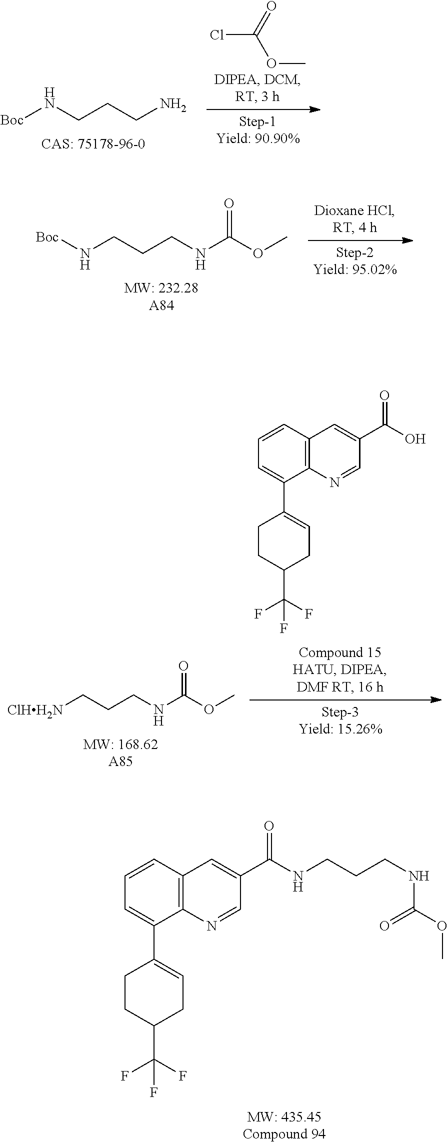

### Step-1

A stirred solution of tert-butyl (3-aminopropyl)carbamate CAS: 75178-96-0 (0.5 g, 2.869 mmol, 1.0 eq.) in DCM (10 mL) was prepared at room temperature. To this reaction mixture, NEt₃ (0.230 g, 2.28 mmol, 0.8 eq.) was added followed by addition of methyl chloroformate (0.4 g, 4.30 mmol, 1.5 eq.) and stirred the reaction mixture at RT for 2 h. TLC indicated the completion of reaction, the resulting reaction mixture was diluted with water (30 mL) and extracted with EtOAc (3×30 mL), organic layer was dried over Na₂SO₄ and evaporated under vacuum to afford tert-butyl methyl propane-1,3-diyldicarbamate (A84) (0.6 g, 2.58 mmol, Yield: 90.90%).

¹H NMR (DMSO-d₆, 400 MHz): δ ppm 7.055 (br s, 1H), 6.76 (br s, 1H), 3.59 (s, 2H), 2.96-2.88 (m, 4H), 1.53-1.42 (m, 2H), 1.37 (s, 9H).

LCMS (Method A): not supported

### Step-2

A stirred solution of tert-butyl methyl propane-1,3-diyldicarbamate (A84) (0.6 g, 2.58 mmol, 1.0 eq.) in DCM (8 mL) was prepared in 30 mL glass vial at room temperature. To this reaction solution, 4M HCl in dioxane (2.38 mL, 7.79 mmol, 3.0 eq.) was added at 0° C. Then stirred the reaction mixture at RT for 4 h. TLC indicated the completion of reaction, then solvent was removed under reduced pressure to afford methyl (3-aminopropyl)carbamate hydrochloride (A85) (0.7 g, 4.151 mmol, Yield: 95.03%).

¹H NMR (DMSO-d₆, 400 MHz): δ ppm 7.95 (br s, 2H) 7.28 (t, J=6.0 Hz, 1H), 3.52 (s, 3H), 3.06-3.03 (m, 2H), 2.78-2.73 (m, 2H), 1.79-1.68 (m, 2H).

LCMS (Method A): not supported

### Step-3

To a stirred solution of 8-(4-(trifluoromethyl)cyclohex-1-en-1-yl)quinoline-3-carboxylic acid (Compound 15) (0.1 g, 0.321 mmol, 1.0 eq.) in DMF (1.0 mL) was added DIPEA (0.15 mL, 0.936 mmol, 3.0 eq.) and HATU (0.177 g, 0.468 mmol, 1.5 eq.) at 0° C. followed by addition of methyl (3-aminopropyl)carbamate hydrochloride (A85) (0.062 g, 0.374 mmol, 1.2 equiv). The resulting reaction mixture was stirred at RT for 16 h. TLC indicated the completion of reaction, the resulting reaction mixture was diluted with cold water (30 mL) and extracted with EtOAc (3×30 mL) organic layer was dried over Na₂SO₄ and evaporated under vacuum and obtained crude was purified by trituration with pentane to afford methyl (2-(8-(4-(trifluoromethyl)cyclohex-1-en-1-yl)quinoline-3-carboxamido)ethyl) carbamate (Compound 94) (0.020 g, 0.045 mmol, Yield: 15.26%)

¹H NMR (DMSO-d₆, 400 MHz): δ ppm 9.24 (d, J=2.4 Hz, 1H), 8.78 (d, J=2 Hz, 2H), 8.00 (q, J=4 Hz, J=5.6 Hz, 1H), 7.63 (t, J=2 Hz, 2H), 7.17 (br s, 1H), 5.86 (br s, 1H), 3.52 (s, 3H) 3.08 (q, J=6.4, J=12.8, 3H), 2.80-2.79 (m, 1H), 2.72-2.67 (m, 2H), 2.33-2.24 (m, 2H), 2.10-2.07 (m, 1H), 1.74-1.65 (m, 3H). Note: —CF₃CH proton merged in DMSO solvent peaks.

¹H NMR (MeOD, 400 MHz): δ ppm 9.25 (d, J=2 Hz, 1H), 8.76 (d, J=2 Hz, 2H), 7.97 (dd, J=2 Hz, 7.6 Hz 1H), 7.69-7.63 (m, 2H), 5.87 (br s, 1H), 3.65 (s, 3H), 3.51 (t, J=6.8, 2H), 3.25 (t, J=6.8 Hz, 2H), 2.78-2.75 (m, 1H), 2.68-2.65 (m, 1H), 2.55-2.51 (m, 1H), 2.40-2.34 (m, 1H), 2.18-2.15 (m, 1H), 1.93-1.82 (m, 3H). Note: —CONH protons are exchanged in MeOD NMR.

LCMS (Method A): 2.362 min, 97.92%, 254.0 nm MS: ES+436.53 [M+H]

HPLC (Method A): 8.31 min, 95.34%, 254.0 nm

### Example 88—Synthesis of N-(3-hydroxypropyl)-8-(4-(trifluoromethyl)cyclohex-1-en-1-yl)quinoline-3-carboxamide (Compound 95)

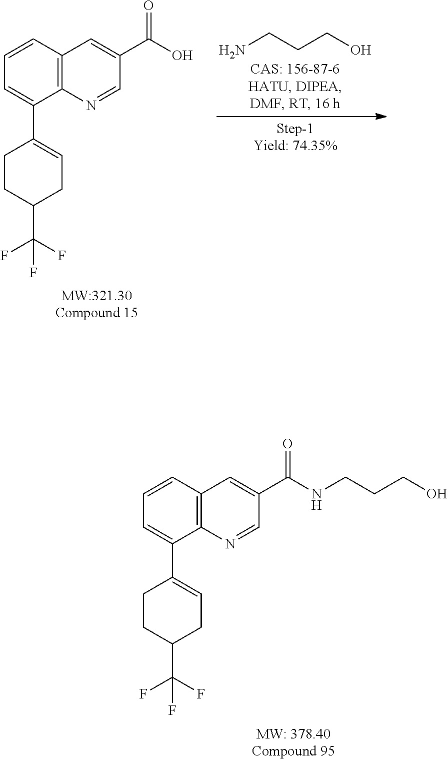 [SMILES: O=C(O)c1cnc2c(C3=CCC(C(F)(F)F)CC3)cccc2c1 ; NCCCO ; O=C(NCCCO)c1cnc2c(C3=CCC(C(F)(F)F)CC3)cccc2c1]

### Step-1

To a stirred solution of 8-(4-(trifluoromethyl)cyclohex-1-en-1-yl)quinoline-3-carboxylic acid (Compound 15) (0.1 g, 0.311 mmol, 1.0 eq.) in DMF (1 mL) was added DIPEA (0.15 mL, 0.936 mmol, 3.0 eq.) and HATU (0.177 g, 0.467 mmol, 1.5 eq.) at room temperature. Followed by addition of CAS: 156-87-6 3-Amino-1-propanol (0.026 g, 0.352 mmol, 1.1 equiv). The resulting reaction mixture was stirred at RT for 16 h. TLC indicated the completion of reaction, the resulting reaction mixture was diluted with cold water (30 mL) and extracted with EtOAc (3×30 mL), organic layer was dried over Na₂SO₃ and evaporated under vacuum. crude was purified by column chromatography using silica gel (230-400 mesh) (eluting with 40% Ethyl Acetate in Hexane) to afford N-(3-hydroxypropyl)-8-(4-(trifluoromethyl)cyclohex-1-en-1-yl)quinoline-3-carboxamide (Compound 95) (0.087 g, 0.229 mmol, Yield: 74.35%)

¹H NMR (DMSO-d₆, 400 MHz): δ ppm 9.24 (d, J=2 Hz, 1H), 8.79 (d, J=1.6 Hz, 2H), 8.01-7.99 (m, 1H), 7.64-7.62 (m, 2H), 5.87 (br s, 1H), 4.52 (t, J=5.2 Hz, 1H), 3.50 (q, J=6 Hz, 2H), 3.41-3.38 (m, 2H), 2.80-2.67 (m, 3H), 2.47 (br s, 1H), 2.30 (d, J=14.4 Hz, 1H), 2.08 (d, J=12 Hz, 1H), 1.76-1.67 (m, 3H).

¹H NMR (MeOD, 400 MHz): δ ppm 9.24 (d, J=2 Hz, 1H), 8.75 (d, J=2 Hz, 1H), 7.97 (dd, J=2 Hz, J=8 Hz, 1H), 7.69-7.63 (m, 2H), 5.86 (br s, 1H), 3.71 (t, J=5.2 Hz, 2H), 3.57 (t, J=7.2 Hz, 2H), 2.81-2.75 (m, 3H), 2.65 (d, J=12.8 Hz, 1H), 2.40-2.33 (m, 1H), 2.17 (t, J=14.4 Hz, 1H), 1.94-1.88 (m, 3H).

Note: —CONH and —OH protons are exchanged in MeOD NMR.

LCMS (Method A): 2.182 min, 99.26%, 254.0 nm, MS: ES+379.32 [M+H]

HPLC (Method A): 7.79 min, 98.27%, 254.0 nm

### Example 89—Synthesis of N-(3-hydroxy-2-methylpropyl)-8-(4-(trifluoromethyl)cyclohex-1-en-1-yl)quinoline-3-carboxamide (Compound 96)

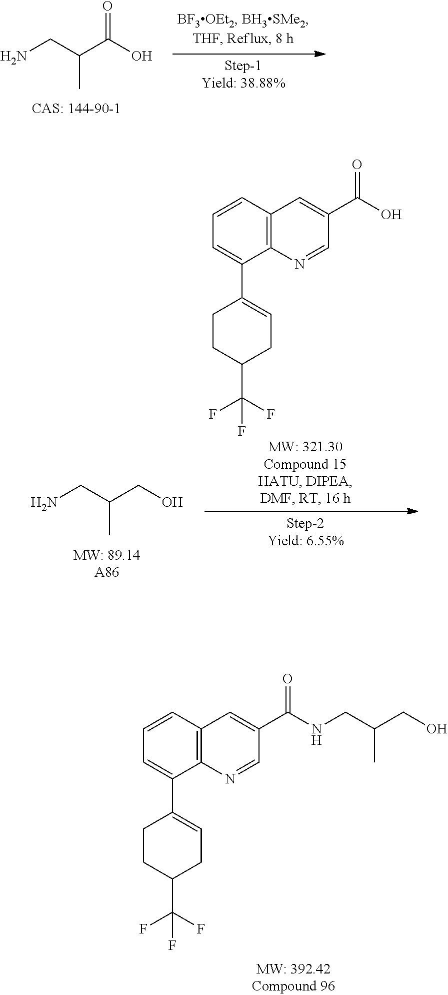

### Step-1

To a stirred solution of 3-amino-2-methylpropanoic acid CAS: 144-90-1 (1.0 g, 9.70 mmol, 1.0 eq.) in THF (10 mL) was added BF₃·OEt₃ (1.3 g, 9.70 mmol, 1.0 eq.) at room temperature. The reaction mixture was heated at reflux temperature for 3 h and then BH₃SMe₂ (0.88 g, 11.64 mmol, 1.2 eq.) was added at RT. Followed by heating the reaction mixture at reflux again for 5 h. TLC indicated the completion of reaction, the resulting reaction mixture was filtered and the filtrate concentrated under vacuum to yield 3-amino-2-methylpropan-1-ol (A86) (1.0 g, 11.21 mmol, Yield: 38.88%).

¹H NMR (DMSO-d₆, 400 MHz): δ ppm 8.04 (br s, 5H), 4.29 (br s, 3H), 3.55 (d, 7.6 Hz, 3H), 3.40-3.38 (m, 3H), 3.36-3.27 (m, 1H), 2.88-2.74 (m, 3H), 2.72-2.70 (m, 1H), 1.16 (d, J=7.2 Hz, 3H), 0.87 (d, J=6.8 Hz, 2H). Note: Proton count is more due to unconsumed SM.

LCMS (Method A): 0.254 min, 43.27%, 254.0 nm, MS: ES+90.01 (M+1)

### Step-2

To a stirred solution of 8-(4-(trifluoromethyl)cyclohex-1-en-1-yl)quinoline-3-carboxylic acid (Compound 15) (0.1 g, 0.321 mmol, 1.0 eq.) in DMF (1 mL) was added HATU (0.177 g, 0.468 mmol, 1.5 eq.) and DIPEA (0.15 mL, 0.936 mmol, 3.0 eq.) at RT under nitrogen atmosphere, followed by addition of (A86) (0.033 g, 0.373 mmol, 1.2 eq.) and the reaction stirred at RT for 16 h. TLC indicated the completion of reaction, the resulting reaction mixture was extracted with cold water (30 mL) and EtOAc (30 mL) three times, organic layer was dried over Na₂SO₃ and evaporated under vacuum to get crude product. The crude was purified by column chromatography using silica gel (230-400 mesh) (eluting with 25% Ethyl Acetate in Hexane) to afford N-(3-hydroxy-2-methylpropyl)-8-(4-(trifluoromethyl)cyclohex-1-en-1-yl)quinoline-3-carboxamide (Compound 96) (0.008 g, 0.204 mmol, Yield: 6.55%)

¹H NMR (DMSO-d₆, 400 MHz): δ ppm 9.24 (s, 1H), 8.79 (m, 2H), 8.01 (t, J=4 Hz, 1H), 7.63 (d, J=4 Hz, 2H), 5.86 (br s, 1H), 4.55 (t, J=5.2 Hz, 1H), 3.21-3.17 (m, 1H), 2.80-2.67 (m, 2H), 2.33-2.20 (m, 1H), 2.10-2.07 (m, 1H), 1.88-1.86 (m, 2H), 1.70-1.68 (m, 2H), 0.90 (d, J=6.8 Hz, 3H). Note: —CH₂ and CF₃CH proton merged in DMSO solvent peak.

¹H NMR (MeOD, 400 MHz): δ ppm 9.24 (d, J=2 Hz, 1H), 8.76 (d, J=2 Hz, 2H), 7.97 (d, J=6 Hz, 1H), 7.69-7.63 (m, 2H), 5.87 (br s, 1H), 3.55 (d, J=5.6 Hz, 2H), 3.51-3.47 (m, 1H), 3.43-3.32 (m, 1H), 2.78-2.75 (m, 1H), 2.66-2.65 (m, 1H), 2.55-2.51 (m, 1H), 2.40-2.37 (m, 1H), 2.17 (d, J=13.2 Hz, 1H), 2.05-2.00 (m, 1H), 1.91-1.85 (m, 1H), 1.04 (d, J=6.8 Hz, 3H). Note: —CONH and —OH protons are exchanged in MeOD NMR.

LCMS (Method A): 2.322 min, 97.12%, 254.0 nm, MS: ES+393.37 (M+1)

HPLC (Method A): 8.22 min, 95.24%, 254.0 nm

### Example 90—Synthesis of (N-(3-hydroxybutyl)-8-(4-(trifluoromethyl)cyclohex-1-en-1-yl)quinoline-3-carboxamide (Compound 97)

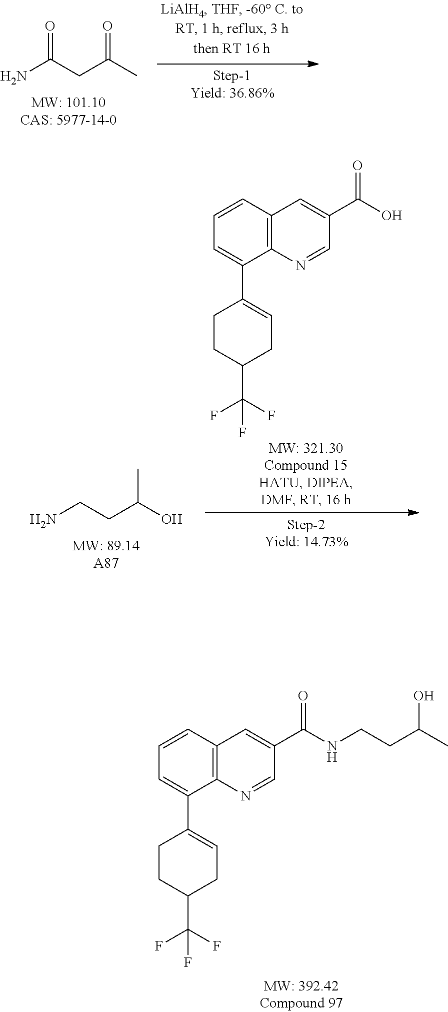

### Step-1

To a stirred solution of 3-oxobutanamide (CAS: 5977-14-0) (2.0 g, 19.78 mmol, 1.0 eq.) in dry THF (20 mL), was added LiAlH₄ solution (2M in THF) (49 mL, 5.0 eq.) slowly at −60° C. under the N₂ atmosphere. The reaction mixture was allowed to warm up to room temperature over the period of 1 h. After 1 h, reaction mixture was refluxed for 3 h. then allowed to cool to room temperature and continued stirring for another 16 h. TLC indicated the completion of reaction, the resulting reaction mixture was quenched with water (20 mL) also added saturated NaHCO₃ solution (40 mL) and extracted with ethyl acetate (5×100 mL). The combined organic layers were dried over Na₂SO₄ and concentrated under reduced pressure to afford 4-aminobutan-2-ol (A87) as yellowish oil. The crude was used for next step without any further purification (0.65 g, 7.29 mmol, Yield 36.86%).

LCMS (Method-A): 0.232 min, 91.53%, 210.0 nm, MS: ES+90.0 (M+1)

### Step-2

To a stirred solution of 8-(4-(trifluoromethyl)cyclohex-1-en-1-yl)quinoline-3-carboxylic acid (Compound 15) (0.15 g, 0.467 mmol, 1.0 eq.) in DMF (1.0 mL), was added HATU (0.26 g, 0.700 mmol, 1.5 eq.) and DIPEA (0.18 g, 1.4 mmol, 3.0 eq.) at 0° C. The reaction mixture was stirred under the N₂ atmosphere. After 15 min of stirring at 0° C. 4-aminobutan-2-ol (A87) (0.045 g, 0.5135 mmol, 1.1 eq.) was added at same temperature and the resulting reaction mixture was stirred at RT for 16 h. TLC indicated the completion of reaction, the resulting reaction mixture was quenched with ice-cold water (3.0 mL) and extracted with ethyl acetate (10 mL×3). The combined organic layers were dried over Na₂SO₄, filtered and concentrated under reduced pressure to afford crude. The crude was purified by column chromatography using silica gel (230-400 Mesh) as stationary phase (40% Ethyl acetate in Hexane) to afford N-(3-hydroxybutyl)-8-(4-(trifluoromethyl)cyclohex-1-en-1-yl)quinoline-3-carboxamide (Compound 97) (0.027 g, 0.069 mmol, Yield 14.73%)

¹H NMR (DMSO, 400 MHz): δ ppm, 9.24 (d, J=2.0 Hz, 1H), 8.78 (m, 2H), 8.0 (dd, J=4.0, 5.6 Hz, 1H), 7.64-7.63 (m, 2H), 5.86 (br s, 1H), 4.50-4.10 (m, 1H), 3.74-3.70 (m, 1H), 3.42-3.33 (m, 2H), 2.72-2.67 (m, 3H), 2.33-2.28 (m, 1H), 2.10-2.07 (m, 1H), 1.71-1.59 (m, 3H), 1.47 (s, 1H), 1.11 (d, J=6.0 Hz, 3H). CF₃—CH proton merged with DMSO solvent peak which is clearly observed in D₂O.

¹H NMR (MeOD, 400 MHz): δ ppm 9.25 (d, J=2.4 Hz, 1H), 8.79 (d, J=2.4 Hz, 1H), 7.98 (dd, J=2.0, 8.0 Hz, 1H), 7.71-7.65 (m, 2H), 5.87 (br s, 1H), 3.93-3.88 (m, 1H), 3.63-3.55 (m, 2H), 2.81-2.65 (m, 3H), 2.65-2.51 (m, 1H), 2.41-2.32 (m, 1H), 2.19-2.13 (m, 1H), 1.90-1.76 (m, 3H), 1.26 (d, J=6.4 Hz, 3H). Note: —CONH and —OH protons are exchanged in MeOD NMR.

LCMS (Method-A): 2.115 min, 99.68%, 254.0 nm, MS: ES+393.4 (M+1)

HPLC (Method-A): 8.09 min, 99.22%, 254 nm.

Chiral HPLC (Method-A): Peak-1: 1.92 min, 22.84%, 242.0 nm; Peak-2: 2.06 min, 26.38%, 242.0 nm; Peak-3: 3.18 min, 25.21%, 242.0 nm; Peak-4: 3.66 min, 24.85%, 242.0 nm.

### Example 91—Synthesis of N—((R)-4-(methylsulfonamido) butan-2-yl)-8-(4-(trifluoromethyl)cyclohex-1-en-1-yl)quinoline-3-carboxamide (Compound 98)

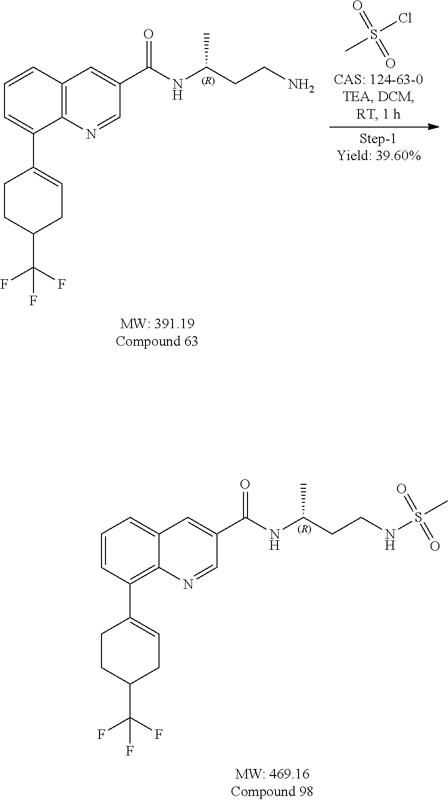

### Step-1

To a stirred solution of N—((R)-4-aminobutan-2-yl)-8-(4-(trifluoromethyl)cyclohex-1-en-1-yl)quinoline-3-carboxamide (Compound 63) (0.04 g, 0.10 mmol, 1.0 eq.) in DCM (1.0 mL) was added TEA (0.031 g, 0.30 mmol, 3.0 eq.) at 0° C. The reaction mixture was stirred at 0° C. for 10 min. After 10 min of stirring at 0° C. methanesulfonyl chloride (CAS: No: 124-63-0) (0.017 g, 0.15 mmol, 1.5 eq.) was added then the resulting reaction mixture was stirred at RT for 1 h. TLC indicated the completion of reaction, the resulting reaction mixture was quenched with ice-cold water (5 mL) and extracted with DCM (10 mL×2). The combined organic layers were dried over Na₂SO₄ and concentrated under reduced pressure to afford crude. The crude material was purified by flash column chromatography using silica (230-400 Mesh) as stationary phase (30-40% Ethyl acetate in Hexane) to afford N—((R)-4-(methylsulfonamido) butan-2-yl)-8-(4-(trifluoromethyl)cyclohex-1-en-1-yl)quinoline-3-carboxamide (Compound 98) (0.019 g, 0.04 mmol, Yield: 39.60%).

¹H NMR (DMSO-d₆, 400 MHz): δ ppm, 9.24 (d, J=2.0 Hz, 1H), 8.79 (d, J=2.0 Hz, 1H), 8.60 (d, J=8.0 Hz, 1H), 8.01 (t, J=4.8 Hz, 1H), 7.64-7.63 (m, 2H), 6.99 (t, J=5.6 Hz, 1H), 5.87 (br s, 1H), 4.17-4.14 (m, 1H), 3.02 (q, J=6.8, 13.2 Hz, 2H), 2.89 (s, 3H), 2.80-2.67 (m, 3H), 2.33-2.28 (m, 1H), 2.10-2.07 (m, 1H), 1.80-1.67 (m, 3H), 1.21 (d, J=6.4 Hz, 3H). Note: CF₃—CH proton merged with DMSO solvent peak which is clearly observed in MeOD.

¹H NMR (MeOD, 400 MHz): δ ppm, 9.23 (d, J=2.0 Hz, 1H), 8.76 (d, J=2.0 Hz, 1H), 7.98-7.96 (m, 1H), 7.69-7.63 (m, 2H), 5.87 (br s, 1H), 4.33 (q, J=6.8, 13.6 Hz, 1H), 3.24-3.18 (m, 2H), 2.95 (s, 3H), 2.78-2.75 (m, 1H), 2.68-2.65 (m, 2H), 2.55-2.51 (m, 1H), 2.40-2.37 (m, 1H), 2.18-2.15 (m, 1H), 1.91-1.83 (m, 2H), 1.35 (d, J=6.4 Hz, 3H). Note: —CONH and —NHSO₂Me proton exchanged in MeOD NMR.

LCMS (Method A): 2.358 min, 100%, 254.0 nm, MS: ES+470.4 (M+1).

HPLC (Method A): 8.13 min, 98.02%, 210 nm.

### Example 92—Synthesis of N-(2-(3-methylureido)ethyl)-8-(4-(trifluoromethylcyclohex-1-en-1-yl)quinoline-3-carboxamide and (Compound 99)

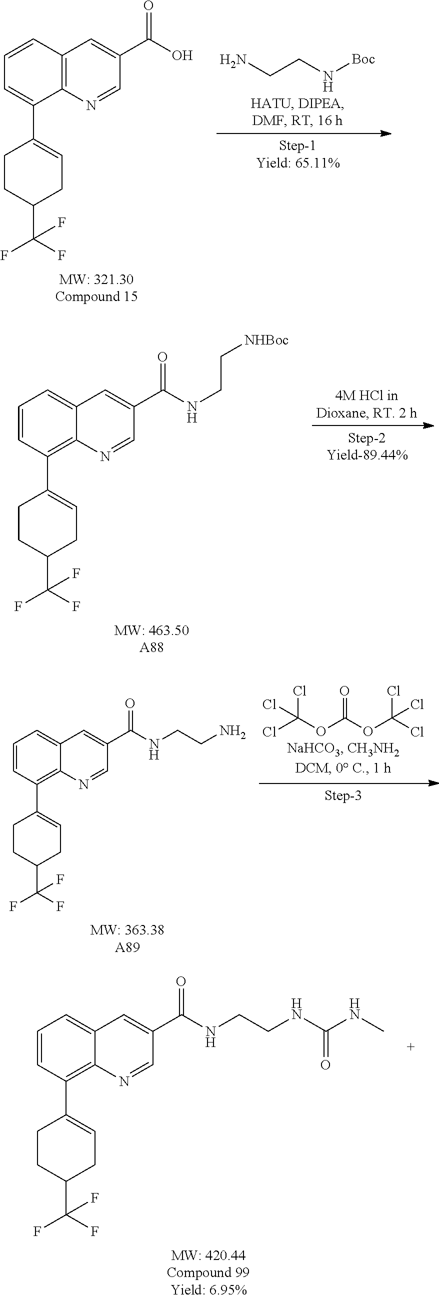 [SMILES: O=C(O)c1cnc2c(C3=CCC(C(F)(F)F)CC3)cccc2c1 ; CC(C)(C)OC(=O)NCCN ; CC(C)(C)OC(=O)NCCNC(=O)c1cnc2c(C3=CCC(C(F)(F)F)CC3)cccc2c1 ; NCCNC(=O)c1cnc2c(C3=CCC(C(F)(F)F)CC3)cccc2c1 ; O=C(OC(Cl)(Cl)Cl)OC(Cl)(Cl)Cl ; CNC(=O)NCCNC(=O)c1cnc2c(C3=CCC(C(F)(F)F)CC3)cccc2c1]

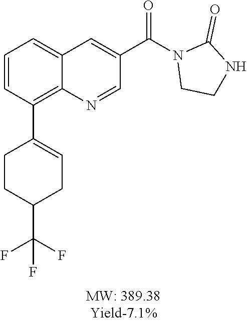 [SMILES: O=C1NCCN1C(=O)c1cnc2c(C3=CCC(C(F)(F)F)CC3)cccc2c1]

### Step-1

To a stirred solution of Compound 15 (0.3 g, 0.93 mmol, 1.0 eq.) and CAS: 57260-73-8 1-Boc-ethylenediamine (0.19 g, 1.23 mmol, 1.3 eq.), in DMF (2 mL) was added HATU (0.53 g, 1.39 mmol, 1.5 eq.), DIPEA (0.48 mL, 2.79 mmol, 3.0 eq.), under the N₂ atmosphere. The resulting mixture was stirred at room temperature for 16 h. TLC indicated the completion of reaction; the resulting reaction mixture was extracted with ethyl acetate (2×30 mL). The combined organic layers were dried over Na₂SO₄ and concentrated under reduced pressure to afford crude. The crude was purified by manual column chromatography using silica (60-120 mesh) as stationary phase (100% EtOAC) to afford tert-butyl (2-(8-(4-(trifluoromethyl)cyclohex-1-en-1-yl)quinoline-3-carboxamido)ethyl) carbamate (A88) (0.28 g, 0.640 mmol, Yield 65.11%)

¹H NMR (DMSO, 400 MHz): δ ppm 9.24 (s, 1H), 8.80 (d, J=13.6 Hz, 2H), 7.97 (d, J=15.2 Hz, 1H), 7.64 (s, 2H), 6.99 (s, 1H), 5.87 (s, 1H), 3.16 (d, J=4.0 Hz, 2H), 2.77-2.69 (m, 4H), 2.32-1.67 (m, 4H), 1.37 (s, 9H), 1.25 (s, 1H).

LCMS (Method A): 2.611 min, 87.31%, 210.0 nm

### Step-2

To a stirred solution of (A88) (0.24 g, 0.51 mmol, 1.0 eq.) in Dioxane (1 mL) was added HCl in Dioxane (1.5 mL) and the reaction mixture was stirred under the N₂ atmosphere for 2 h. TLC indicated the completion of reaction, the reaction mixture was concentrated under reduced pressure to afford N-(2-aminoethyl)-8-(4-(trifluoromethyl)cyclohex-1-en-1-yl)quinoline-3-carboxamide (A89) (0.21, 0.57 mmol, Yield 89.44%)

¹H NMR (DMSO, 400 MHz): δ ppm 9.31 (d, J=2.4 Hz, 1H), 9.11 (t, J=5.2 Hz, 1H), 8.91 (d, J=2.0 Hz, 1H), 8.03-8.01 (m, 4H), 7.66 (t, J=8.0 Hz, 2H), 5.87 (s, 1H), 3.60-3.07 (m, 5H), 2.89-2.69 (m, 6H), 2.29-2.07 (m, 2H), 1.72-1.67 (m, 1H), 1.29-1.23 (m, 2H). Note: Proton count is more due to HCl salt.

### Step-3

To a stirred solution of (A89) (0.210 g, 0.57 mmol, 1.0 eq.) in DCM (2.0 mL) was added NaHCO₃ (0.048 g, 0.57 mmol, 1.0 eq.) and Triphosgene (0.050 g, 0.18 mmol, 0.32 eq.) followed by addition of CH₃NH₂ 2M in THF (2.0 mL, 1.5 eq.). The reaction mixture was stirred at 0° C. for 1 h under N₂ atmosphere. TLC indicated the completion of reaction; the resulting reaction mixture was diluted with water (20 ml) extracted with ethyl acetate (2×30 mL). The combined organic layers were dried over Na₂SO₄ and concentrated under reduced pressure to afford crude. The crude was purified by column chromatography (using 100-200 silica gel), desired product eluted in 50% (DCM:MeOH) to afford 1-(8-(4-(trifluoromethyl)cyclohex-1-en-1-yl)quinoline-3-carbonyl) imidazolidin-2-on (0.016 g, 0.041 mmol, Yield 6.95%); and N-(2-(3-methylureido)ethyl)-8-(4-(trifluoromethyl)cyclohex-1-en-1-yl)quinoline-3-carboxamide (Compound 99) (0.010 g, Yield: 0.025 mmol, 7.1%)

¹H NMR (MeOD, 400 MHz): δ ppm 9.25 (d, J=2.4 Hz, 1H), 8.76 (d, J=2.0 Hz, 1H), 7.97 (dd, J=8.0, 2.0 Hz, 1H), 7.69-7.63 (m, 2H), 5.86 (br s, 1H), 3.56-3.44 (m, 2H), 2.81-2.73 (m, 6H), 2.55-2.51 (m, 2H), 2.16 (d, J=10.4 Hz, 1H), 1.89-1.85 (m, 1H). Note: —CONH protons are exchanged in MeOD NMR.

¹H NMR (DMSO, 400 MHz): δ ppm 9.25 (d, J=2.4 Hz, 1H), 8.90-8.87 (m, 1H), 8.79 (d, J=2.0 Hz, 1H), 8.01-7.98 (m, 1H), 7.65-7.63 (m, 2H), 6.10 (t, J=5.2 Hz, 1H), 5.86 (br s, 2H), 3.28-3.21 (m, 2H), 2.80-2.67 (m, 4H), 2.55-2.54 (m, 3H), 2.32-2.24 (m, 1H), 2.10-2.07 (m, 1H), 1.73-1.67 (m, 1H). Note: —CF₃CH proton merged in DMSO solvent peak.

LCMS (Method A): 2.166 min, 100%, 242.0 nm

HPLC (Method A): 7.37 min, 100%, 210.0 nm

Chiral HPLC-Peak1: 6.51 min, 48.12%: Peak-2: 6.64 min, 51.87%, 240 nm Undesired product:

¹H NMR (MeOD, 400 MHz): δ ppm 8.96 (d, J=2.0 Hz, 1H), 8.54 (d, J=2.0 Hz, 1H), 7.94 (dd, J=7.6, 2.0 Hz, 1H), 7.67-7.59 (m, 2H), 5.85 (br s, 1H), 4.17-4.13 (m, 2H), 3.62-3.58 (m, 2H), 2.79-2.50 (m, 4H), 2.40-2.33 (m, 1H), 2.17-2.14 (m, 1H), 1.93-1.82 (m, 1H). Note: —CONH proton exchanged in MeOD NMR.

¹H NMR (DMSO, 400 MHz): δ ppm 8.91 (d, J=2.4 Hz, 1H), 8.50 (d, J=2.0 Hz, 1H), 7.99 (dd, J=7.2, 6.8 Hz, 1H), 7.83 (s, 1H), 7.63-7.58 (m, 2H), 5.85 (br s, 1H), 4.00 (t, J=7.6 Hz, 2H), 3.45 (t, J=8.0 Hz, 2H), 2.80-2.67 (m, 2H), 2.33-2.28 (m, 1H), 2.10-2.07 (m, 1H), 1.71-1.67 (m, 1H). Note: —CF₃CH proton merged in DMSO solvent peak.

LCMS (Method A): 2.32 min, 96.62%, 254.0 nm, M+H=390.32

HPLC (Method A): 8.19 min, 98.33%, 254.0 nm

### Example 93—Synthesis of N-(3-(3-methylureido) propyl)-8-(4-(trifluoromethyl)cyclohex-1-en-1-yl)quinoline-3-carboxamide (Compound 100)

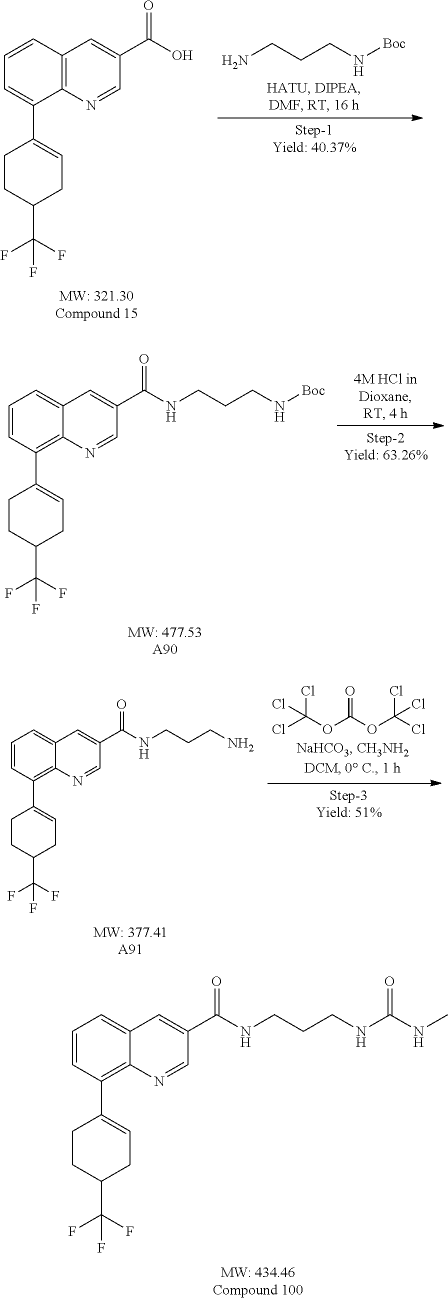 [SMILES: O=C(O)c1cnc2c(C3=CCC(C(F)(F)F)CC3)cccc2c1 ; CC(C)(C)OC(=O)NCCCN ; CC(C)(C)OC(=O)NCCCNC(=O)c1cnc2c(C3=CCC(C(F)(F)F)CC3)cccc2c1 ; NCCCNC(=O)c1cnc2c(C3=CCC(C(F)(F)F)CC3)cccc2c1 ; O=C(OC(Cl)(Cl)Cl)OC(Cl)(Cl)Cl ; CNC(=O)NCCCNC(=O)c1cnc2c(C3=CCC(C(F)(F)F)CC3)cccc2c1]

### Step-1

To a stirred solution of 8-(4-(trifluoromethyl)cyclohex-1-en-1-yl)quinoline-3-carboxylic acid (Compound 15) (0.2 g, 0.62 mmol, 1.0 eq.) in DMF (2.0 mL), was added HATU (0.350 g, 0.93 mmol, 1.5 eq.) and DIPEA (0.31 mL, 1.86 mmol, 3.0 eq.) at 0° C. After 15 min of stirring at 0° C., tert-butyl (3-aminopropyl)carbamate (CAS: No: 75178-96-0) (0.140 g, 0.89 mmol, 1.3 eq.) was added and the resulting reaction mixture was stirred at RT for 16 h. TLC indicated the completion of reaction, the resulting reaction mixture was quenched with ice-cold water (10 mL) and extracted with EtOAc (20 mL×3). The combined organic layers were dried over Na₂SO₄ and concentrated under reduced pressure to afford crude. The crude was purified by manual column chromatography using silica gel (60-120 Mesh) as a stationary phase (100% EtOAC) to afford tert-butyl (3-(8-(4-(trifluoromethyl)cyclohex-1-en-1-yl)quinoline-3-carboxamido)propyl)carbamate (A90) (0.120 g, 0.25 mmol, Yield: 40.37%).

¹H NMR (DMSO, 400 MHz): δ ppm 9.24 (d, J=2.0 Hz, 1H), 8.79-8.76 (m, 2H), 8.01-7.99 (m, 1H), 7.65-7.61 (m, 2H), 6.87 (t, J=5.6 Hz, 1H), 5.86 (br s, 1H), 3.02-3.00 (m, 2H), 2.69-2.60 (m, 5H), 2.33-2.32 (m, 1H), 2.10-2.09 (m, 1H), 1.71-1.66 (m, 4H), 1.37-1.33 (m, 9H).

LCMS (Method A): 2.72 min, 94.87%, 245.0 nm

### Step-2

To a stirred solution of tert-butyl (3-(8-(4-(trifluoromethyl)cyclohex-1-en-1-yl)quinoline-3-carboxamido)propyl)carbamate (A90) (0.120 g, 0.25 mmol, 1.0 eq.) in 1,4-dioxane (2.0 mL), was added 4M HCl in Dioxane (2.0 mL) at 0° C. under N₂ atmosphere. The resulting reaction mixture was stirred at room temperature for 4 h. TLC indicated the completion of reaction, the resulting mixture was concentrated under reduced pressure to afford N-(3-aminopropyl)-8-(4-(trifluoromethyl)cyclohex-1-en-1-yl)quinoline-3-carboxamide (A91) (0.060 g, 0.15 mmol, Yield 63.26%)

¹H NMR (DMSO, 400 MHz): δ ppm 9.29 (d, J=2.4 Hz, 1H), 9.07 (t, J=5.6 Hz, 1H), 8.87 (d, J=2.0 Hz, 1H), 8.03-8.00 (m, 1H), 7.89 (br s, 3H), 7.67-7.63 (m, 2H), 3.44-3.39 (m, 2H), 2.92-2.67 (m, 2H), 2.57-2.50 (m, 3H), 2.42-2.33 (m, 1H), 2.32-2.28 (m, 1H), 1.90-1.69 (m, 2H), 1.67-1.23 (m, 1H). Note: Proton count is more due to HCl Salt.

### Step 3

To a stirred solution of N-(3-aminopropyl)-8-(4-(trifluoromethyl)cyclohex-1-en-1-yl)quinoline-3-carboxamide (A91) (0.250 g, 0.66 mmol, 1.0 eq.) in DCM (2.5 mL), was added NaHCO₃ (0.250 g, 0.66 mmol, 1.0 eq.) and Triphosgene (0.062 g, 0.21 mmol, 0.32 eq.) in DCM (2 mL), followed by addition of CH₃NH₂ 2M in THF (2.0 mL, 1.5 eq.) and the reaction mixture was stirred at 0° C. for 1 h. TLC indicated the completion of reaction; the resulting reaction mixture was extracted with ethyl acetate (2×30 mL). The combined organic layers dried over Na₂SO₄ and concentrated under reduced pressure to afford crude. The crude was purified by reverse phase HPLC to afford N-(3-(3-methylureido) propyl)-8-(4-(trifluoromethyl)cyclohex-1-en-1-yl)quinoline-3-carboxamide (Compound 100) (0.106 g, 0.243 mmol, Yield 51%)

¹H NMR (MeOD, 400 MHz): δ ppm 9.26 (d, J=2.0 Hz, 1H), 8.77 (d, J=2.0 Hz, 1H), 7.98 (dd, J=1.6, 7.6 Hz, 1H), 7.69-7.63 (m, 2H), 5.86 (br s, 1H), 3.52 (t, J=6.8 Hz, 2H), 3.27 (t, J=6.8 Hz, 2H), 2.78-2.76 (m, 1H), 2.72 (s, 3H), 2.69-2.65 (m, 2H), 2.52-2.50 (m, 1H), 2.40-2.37 (m, 1H), 2.17-2.15 (m, 1H), 1.89-1.81 (m, 1H). Note: —CONH protons exchanged in MeOD NMR.

¹H NMR (DMSO, 400 MHz): δ ppm 9.24 (d, J=2.4 Hz, 1H), 8.84-8.78 (m, 2H), 8.02-7.99 (m, 1H), 7.64-7.63 (m, 2H), 5.96 (t, J=5.6 Hz, 1H), 5.87 (br s, 1H), 5.80 (d, J=4.4 Hz, 1H), 3.09-3.07 (d, J=6.4 Hz, 2H), 2.80-2.67 (m, 3H), 2.51-2.59 (m, 6H), 2.33-2.29 (m, 1H), 2.12-2.16 (m, 1H), 1.67-1.64 (m, 3H).

HPLC (Method A)-7.53 min, 95.33%, 215.0 nm

LCMS (Method A)-2.17 min, 100%, 242.0 nm

### Example 94—Synthesis of N-(2-((N-methylsulfamoyl)amino)ethyl)-8-(4-(trifluoromethyl)cyclohex-1-en-1-yl)quinoline-3-carboxamide (Compound 101)

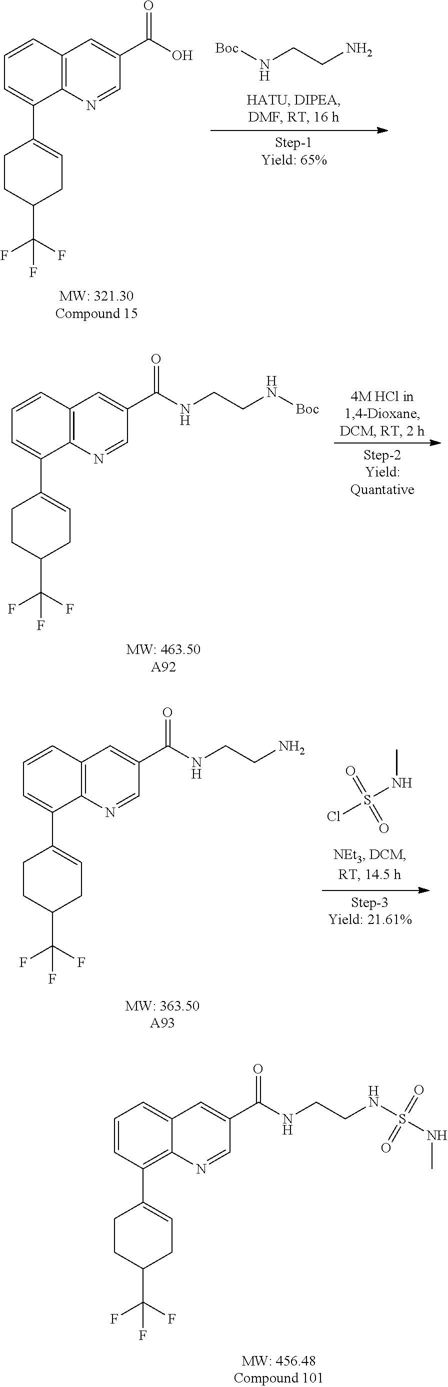

### Step 1

To a stirred solution of 8-(4-(trifluoromethyl)cyclohex-1-en-1-yl)quinoline-3-carboxylic acid (Compound 15) (0.2 g, 0.623 mmol, 1.0 eq.) in DMF (5.0 mL) was added DIPEA (0.31 mL, 1.869 mmol, 3.0 eq.) and HATU (0.16 g, 0.934 mmol, 1.5 eq.) at 0° C. After 15 min of stirring at 0° C., tert-butyl (2-aminoethyl) carbamate (CAS: No: 57260-73-8) (0.119 g, 0.747 mmol, 1.2 eq.) was added and resulting reaction mixture was stirred at RT for 16 h. TLC indicated the completion of reaction; the resulting reaction mixture was quenched with ice-cold water (10 mL) and extracted with EtOAc (20 mL×3). The combined organic layers were dried over Na₂SO₄ and concentrated under reduced pressure to afford crude. The crude material was purified by flash column chromatography using silica (230-400 Mesh) as stationary phase (80% EtOAc in Hexane) to afford tert-butyl (2-(8-(4-(trifluoromethyl)cyclohex-1-en-1-yl)quinoline-3-carboxamido)ethyl) carbamate (A92) (0.190 g, 0.410 mmol, Yield: 65%).

¹H NMR (DMSO-d₆, 400 MHz): δ ppm, 9.24 (d, J=2.4 Hz, 1H), 8.81 (t, J=5.6 Hz, 1H), 8.79 (d, J=2.0 Hz, 1H), 8.0-7.95 (m, 2H), 7.65-7.63 (m, 2H), 7.00-6.97 (t, J=5.6 Hz, 1H), 5.87 (s, 1H), 3.37-3.23 (m, 2H), 2.77-249 (m, 3H), 2.47-1.67 (m, 2H), 2.33-2.28 (m, 1H), 2.10-2.07 (m, 1H), 1.71-1.67 (m, 1H), 1.37 (s, 9H).

LCMS (Method A): 2.639 min, 97.79%, 254.0 nm, MS: ES+464.53 (M+H)

### Step-2

To a solution of tert-butyl (2-(8-(4-(trifluoromethyl)cyclohex-1-en-1-yl)quinoline-3-carboxamido)ethyl) carbamate (A92) (0.190 g, 0.410 mmol 1.0 eq.) in DCM (2.0 mL), was added 4M HCl in 1,4-dioxane (1.9 mL, 10 V) at 0° C. under N₂ atmosphere. The resulting reaction mixture was stirred at room temperature for 2 h. TLC indicated the completion of reaction; the reaction mixture was concentrated under reduced pressure to afford crude which was triturated with ether (5 mL×2) to yield N-(2-aminoethyl)-8-(4-(trifluoromethyl)cyclohex-1-en-1-yl)quinoline-3-carboxamide (A93) (0.180 g, 0.495 mmol, Yield: quantitative).

¹H NMR (DMSO-d₆, 400 MHz): δ ppm, 9.31 (d, J=2.0 Hz, 1H), 9.11 (t, J=5.2 Hz, 1H), 8.91 (d, J=1.6 Hz, 1H), 8.03-8.01 (m, 3H), 7.67-7.64 (m, 2H), 5.87 (br s, 1H), 3.62-3.57 (m, 2H), 3.06-3.03 (m, 2H), 2.80-2.67 (m, 4H), 2.33-2.25 (m, 1H), 2.10-2.07 (m, 1H), 1.75-1.66 (m, 1H).

LCMS (Method A): 1.853 min, 97.39%, 254.0 nm, MS: ES+364 (M+1)

### Step-3

To a solution N-(2-aminoethyl)-8-(4-(trifluoromethyl)cyclohex-1-en-1-yl)quinoline-3-carboxamide (A93) (0.140 g, 0.385 mmol, 1.0 eq.) in DCM (3.0 mL), was added TEA (0.13 mL, 0.77 mmol, 2.0 eq.) at 0° C. The reaction mixture was stirred at 0° C. for 10 min. followed by addition of methyl sulfamoyl chloride (0.490 g, 3.85 mmol, 10 eq.) and the resulting reaction mixture was stirred at 0° C. for 30 min and room temperature for 14.5 h. TLC indicated the completion of reaction; the resulting reaction mixture was quenched with ice-cold water (10 mL) and extracted with EtOAc (20 mL×3). The combined organic layers were dried over Na₂SO₄ and concentrated under reduced pressure to afford crude. The crude material was purified by flash column chromatography using silica (230-400 mesh) as stationary phase (70% EtOAc in Hexane) to afford N-(2-((N-methylsulfamoyl)amino)ethyl)-8-(4-(trifluoromethyl)cyclohex-1-en-1-yl)quinoline-3-carboxamide (Compound 101) (0.038 g, 0.083 mmol, Yield: 21.61%).

¹H NMR (DMSO-d₆, 400 MHz): δ ppm, 9.25 (d, J=2.0 Hz, 1H), 8.86 (t, J=5.6 Hz, 1H), 8.80 (d, J=2.0 Hz, 1H), 8.0 (dd, J=4.0, 6.0 Hz, 1H), 7.65-7.63 (m, 2H), 7.06 (t, J=5.6 Hz, 1H), 6.75 (m, 1H), 5.87 (br s, 1H), 3.47-3.43 (m, 2H), 3.06-3.01 (m, 2H), 2.80.2.62 (s, 3H), 2.45 (d, J=3.6 Hz, 3H), 2.32-2.25 (m, 1H), 2.10-2.07 (m, 1H), 1.74-1.67 (m, 1H). Note: CF₃—CH proton merged with DMSO solvent peak which is clearly observed in MeOD.

¹H NMR (MeOD, 400 MHz): δ ppm, 9.25 (d, J=2.4 Hz, 1H), 8.78 (d, J=2.0 Hz, 1H), 7.96 (dd, J=2.0, 7.6 Hz, 1H), 7.69-7.63 (m, 2H), 5.86 (br s, 1H), 3.62 (t, J=6.4 Hz, 2H), 3.26 (t, J=6.0 Hz, 2H), 2.78-2.68 (m, 2H), 2.63 (s, 3H), 2.55-2.51 (m, 1H), 2.40-2.33 (m, 1H), 2.17-2.15 (m, 1H), 1.92-1.83 (m, 1H). Note: —CONH protons are exchanged in MeOD NMR.

LCMS (Method A): 2.385 min, 100%, 254.0 nm, MS: ES+457.5 (M+1)

HPLC (Method A): 7.92 min, 98.67%, 254.0 nm.

Chiral HPLC: Peak-1: 4.06 min, 50.83.70%, 242.0 nm; Peak-2: 4.71 min, 49.16%, 242.0 nm.

### Example 95—Synthesis of N-(3-((N-methylsulfamoyl)amino)propyl)-8-(4-(trifluoromethyl)cyclohex-1-en-1-yl)quinoline-3-carboxamide (Compound 102)

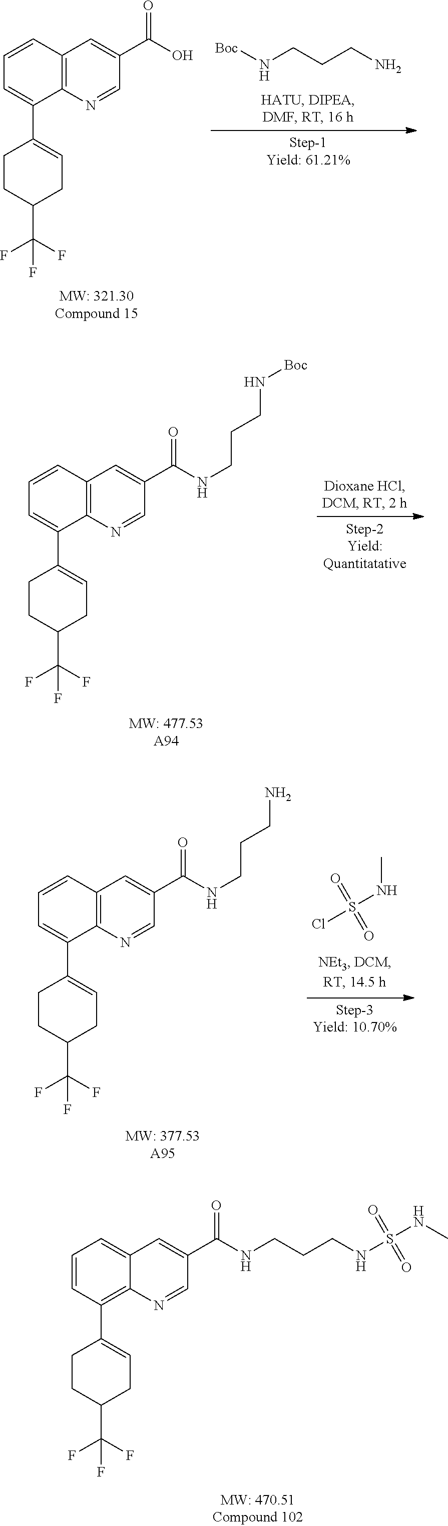

### Step-1

To a stirred solution of 8-(4-(trifluoromethyl)cyclohex-1-en-1-yl)quinoline-3-carboxylic acid (Compound 15) (0.2 g, 0.623 mmol, 1.0 eq.) in DMF (2.0 mL) were added DIPEA (0.3 mL, 1.86 mmol, 3.0 eq.) and HATU (0.350 g, 0.934 mmol, 1.5 eq.) at 0° C. followed by addition of tert-butyl (3-aminopropyl)carbamate (CAS: 75178-96-0) (0.129 g, 0.747 mmol, 1.2 eq.) and the resulting reaction mixture was stirred at RT for 16 h. TLC indicated the completion of reaction; the resulting reaction mixture was quenched with ice-cold water (10 mL) and extracted with EtOAc (20 mL×3). The combined organic layers were dried over Na₂SO₄ and concentrated under reduced pressure to afford crude. The crude material was purified by flash column chromatography using silica (230-400 Mesh) as stationary phase (80% EtOAc in Hexane) to afford tert-butyl (3-(8-(4-(trifluoromethyl)cyclohex-1-en-1-yl)quinoline-3-carboxamido)propyl)carbamate (A94) (0.185 g, 0.387 mmol, Yield: 61.21%).

¹H NMR (DMSO-d₆, 400 MHz): δ ppm, 9.23 (d, J=2.4 Hz, 1H), 8.78-8.76 (m, 2H), 8.02-7.98 (m, 1H), 7.65-7.61 (m, 2H), 6.86 (t, J=5.6 Hz, 1H), 5.86 (br s, 1H), 3.04-2.99 (m, 2H), 2.80-2.67 (m, 3H), 2.33-2.32 (m, 1H), 2.10-2.06 (m, 1H), 1.73-1.65 (m, 3H), 1.37 (s, 9H).

Note: CH₂ proton merged with DMSO moisture peak which is clearly observed in D₂O and CF₃—CH proton merged with DMSO solvent peak which is clearly observed in D₂O.

LCMS (Method A): 2.725 min, 84.36%, 210.0 nm, MS: ES+478.54 (M+1)

### Step-2

To a solution of tert-butyl (3-(8-(4-(trifluoromethyl)cyclohex-1-en-1-yl)quinoline-3-carboxamido)propyl)carbamate (A94) (0.185 g, 0.387 mmol 1.0 eq.) in DCM (1.8 mL) was added 4 M HCl in 1,4-dioxane (1.8 mL, 10 v) at 0° C. under N₂ atmosphere. The resulting reaction mixture was stirred at room temperature for 2 h. TLC indicated the completion of reaction; the reaction mixture was concentrated under reduced pressure to obtain the residue. The crude material was triturated with ether (5.0 mL×2) to afford N-(3-aminopropyl)-8-(4-(trifluoromethyl)cyclohex-1-en-1-yl)quinoline-3-carboxamide hydrochloride (A95) (0.16 g, 0.424 mmol, Yield: Quantitative).

¹H NMR (DMSO-d₆, 400 MHz): δ ppm, 9.29 (d, J=2.4 Hz, 1H), 9.09 (t, J=5.6 Hz, 1H), 8.88 (d, J=2.4 Hz, 1H), 8.04-8.00 (m, 1H), 7.92 (br s, 3H), 7.68-7.64 (m, 2H), 5.87 (br s, 1H), 3.44-3.39 (q, J=7.2, 13.0 Hz, 2H), 2.92-2.87 (m, 2H), 2.79-2.67 (m, 2H) 2.33-2.25 (m, 1H), 2.10-2.07 (m, 1H), 1.91-1.83 (m, 2H), 1.74-1.68 (m, 1H). Note: CF₃—CH proton merged with DMSO solvent peak which is clearly observed in D₂O.

LCMS (Method A): 1.86 min, 97.95%, 210.0 nm, MS: ES+378 (M+1)

### Step-3

To a solution N-(3-aminopropyl)-8-(4-(trifluoromethyl)cyclohex-1-en-1-yl)quinoline-3-carboxamide hydrochloride (A95) (0.120 g, 0.318 mmol, 1.0 eq.) in DCM (3.0 mL), was added TEA (0.01 mL, 0.636 mmol, 2.0 eq.) at 0° C. and stirred for 10 min. Followed by addition of methylsulfamoyl chloride (0.41 g, 3.1 mmol, 10 eq.) and reaction mixture was stirred at RT for 14.5 h. TLC indicated the completion of reaction; the resulting reaction mixture was quenched with ice-cold water (10 mL) and extracted with EtOAc (20 mL×3). The combined organic layers were dried over Na₂SO₄ and concentrated under reduced pressure to afford crude. The crude material was purified by flash column chromatography using silica (230-400 Mesh) as stationary phase (70% EtOAc in Hexane) to afford N-(3-((N-methylsulfamoyl)amino)propyl)-8-(4-(trifluoromethyl)cyclohex-1-en-1-yl)quinoline-3-carboxamide (Compound 102) (0.016 g, 0.034 mmol, Yield: 10.70%).

¹H NMR (MeOD, 400 MHz): δ ppm, 9.24 (d, J=2.0 Hz, 1H), 8.76 (d, J=2.0 Hz, 1H), 7.98-7.96 (d, J=7.6 Hz, 1H), 7.69-7.63 (m, 2H), 5.87 (br s, 1H), 3.57 (t, J=6.8 Hz, 2H), 3.09 (t, J=6.8 Hz, 2H), 2.79-2.68 (m, 3H), 2.63 (s, 3H), 2.55-2.51 (m, 1H), 2.41-2.37 (m, 1H), 2.16-2.15 (m, 1H), 1.96-1.85 (m, 3H). Note: Exchangeable proton are exchanged in MeOD NMR.

¹H NMR (DMSO-d₆, 400 MHz): δ ppm, 9.24 (d, J=2.0 Hz, 1H), 8.81-8.78 (m, 2H), 8.02-7.99 (m, 1H), 7.64-7.61 (m, 2H), 6.88 (t, J=6.0 Hz, 1H), 6.71-6.69 (m, 1H), 5.87 (br s, 1H), 2.92-2.87 (m, 2H), 2.80-2.77 (m, 1H), 2.72-2.67 (m, 1H), 2.46-2.44 (m, 4H), 2.33-2.52 (m, 1H), 2.10-2.06 (m, 1H), 1.81-1.75 (m, 2H), 1.71-1.65 (m, 1H). Note: CF₃—CH proton merged with DMSO solvent peak and CH₂ proton merged with DMSO moisture peak which is clearly observed in MeOD spectrum.

LCMS (Method A): 2.34 min, 100%, 254.0 nm, MS: ES+471.5 (M+1)

HPLC (Method A): 8.00 min, 100%, 254.0 nm

CHIRAL HPLC (Method A): Peak-1: 3.07 min, 49.43%, 240.0 nm; Peak-2: 3.61 min, 50.0%, 240.0 nm.

### Example 96—Chiral separation of N—((S)-1-hydroxy-3-methoxypropan-2-yl)-8-(4-(trifluoromethyl)cyclohex-1-en-1-yl)quinoline-3-carboxamide (Compound 103 and Compound 104)

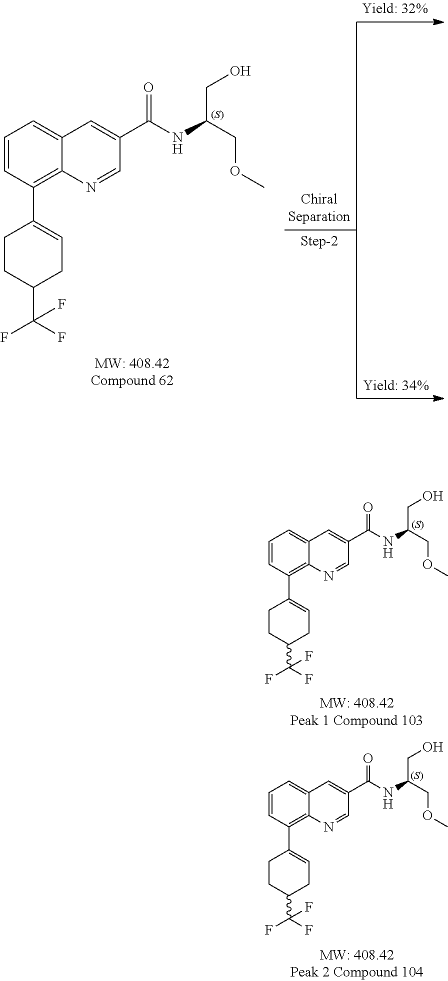

Step-2: Chiral Separation of Compound 103 and Compound 104: N—((S)-1-hydroxy-3-methoxypropan-2-yl)-8-(4-(trifluoromethyl)cyclohex-1-en-1-yl)quinoline-3-carboxamide

COLUMN ID: YMC CELLULOSE, 250×19 mm 5 um

MOBILE PHASE A: 0.1% M·NH3 IN n-HEPTANE

MOBILE PHASE B: 0.1% M·NH3 IN IPA-ACN (50:50)

FLOW RATE (ML/MIN): 32

INSTRUMENT ID: PHP-04 AGILENT 1260 INFINITY-II SERIES WITH UV DETECTOR

METHOD: TIME: FLOW: % A: % B (0.00:32:87:13), (39:32:87:13)

Input Quantity: 0.05 g.

Output Quantity: Compound 103=0.0165 g (% Yield=32%), Compound 104, =0.017 g (% Yield=34%)

### Compound 103:

¹H NMR (DMSO-d₆, 400 MHz): δ ppm, 9.25 (d, J=2.4 Hz, 1H), 8.82 (d, J=2.04 Hz, 1H), 8.83 (d, J=8.0 Hz, 1H), 8.01-7.98 (m, 1H), 7.64-7.61 (m, 2H), 5.87 (br s, 1H), 4.84 (t, J=11.6 Hz, 1H), 4.23-4.18 (m, 1H), 3.57-3.50 (m, 1H), 3.76-2.65 (m, 4H), 2.51 (s, 1H), 2.10-2.07 (m, 1H), 2.46-2.07 (m, 5H), 1.74-1.67 (m, 1H). 1.26-1.23 (m, 1H).

¹H NMR (MeOD, 400 MHz): δ ppm, 9.25 (d, J=1.6 Hz, 1H), 8.79 (d, J=2.04 Hz, 1H), 7.97 (dd, J=7.6 Hz, J=7.6 Hz, 1H), 7.69-7.63 (m, 2H), 5.87 (s, 1H), 4.84 (t, J=11.6 Hz, 1H), 4.40-4.37 (m, 1H), 3.67-3.63 (m, 2H), 3.42-3.32 (m, 2H), 3.21 (s, 1H), 2.78-2.35 (m, 6H), 1.91-1.85 (m, 1H), 1.30 (s, 3H). Note: —CONH and —OH proton exchanged in MeOD NMR.

LCMS (Method A): 2.192 min, 100%, 254.0 nm, MS: ES+409 (M+1)

HPLC (Method A): 7.82 min, 99.74%, 254.0 nm

Chiral HPLC: 5.44 min, %, 240.0 nm

### Compound 104:

¹H NMR (DMSO-d₆, 400 MHz): δ ppm, 9.25 (d, J=2.0 Hz, 1H), 8.79 (d, J=2.4 Hz, 1H), 8.82 (dd, J=8.0 Hz, 1H), 8.0-7.99 (m, 1H), 7.64-7.61 (m, 2H), 5.87 (br s, 1H), 4.84 (t, J=6.0 Hz, 1H), 4.21-4.18 (m, 1H), 3.55-3.28 (m, 4H), 2.51 (s, 3H), 2.10-2.07 (m, 1H), 2.51-2.01 (m, 3H), 1.71-1.67 (m, 1H).

¹H NMR (MeOD, 400 MHz): δ ppm, 9.25 (d, J=2.0 Hz, 1H), 8.27 (d, J=2.0 Hz, 1H), 7.97 (dd, J=7.6 Hz, J=7.6 Hz, 1H), 7.69-7.63 (m, 2H), 5.87 (br s, 1H), 4.40-4.37 (t, J=6.0 Hz, 1H), 3.55-3.28 (m, 4H), 2.51 (s, 3H), 2.76-2.51 (m, 4H), 2.41-2.37 (m, 1H), 2.18-2.15 (m, 1H), 1.93-1.85 (m, 1H). Note: —CONH and —OH proton exchanged in MeOD NMR.

LCMS (Method A): 2.194 min, 100%, 254.0 nm, MS: ES+409 (M+1)

HPLC (Method A): 7.82 min, 100%, 210.0 nm

Chiral HPLC: 6.19 min, 96%, 240.0 nm

### Example 97—Synthesis of (2R)-2-(8-(4-(trifluoromethyl)cyclohex-1-en-1-yl)quinoline-3-carboxamido)propyl dihydrogen phosphate (Compound 105)

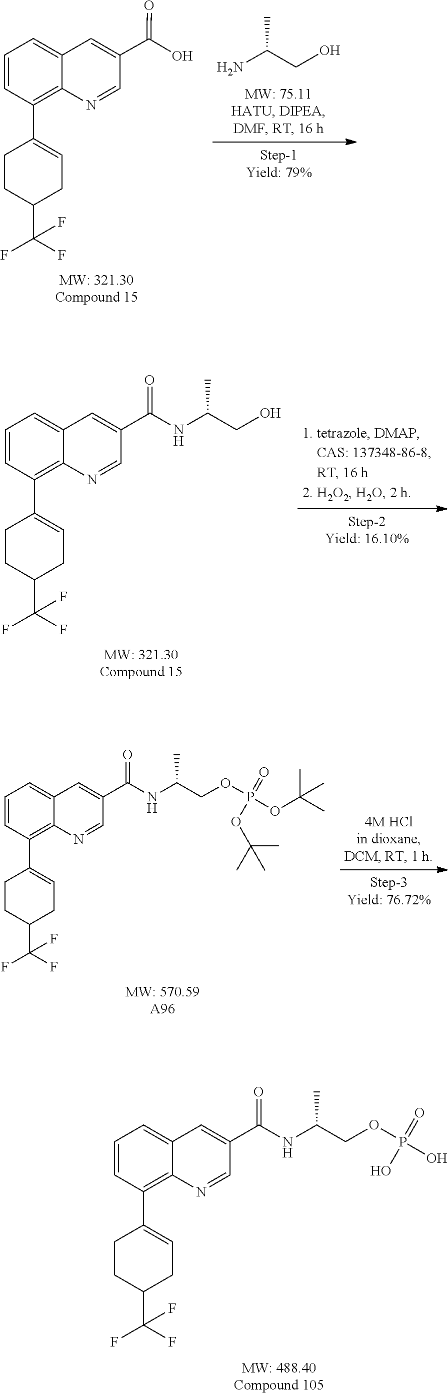

### Step-1

To a stirred solution of 8-(4-(trifluoromethyl)cyclohex-1-en-1-yl)quinoline-3-carboxylic acid (Compound 15) (1.0 g, 3.113 mmol, 1.0 eq.) in DMF (10 mL), was added CAS: 35320-23-1 D-Alaninol (0.234 g, 3.113 mmol, 1.0 eq.), DIPEA (1.2 g, 9.339 mmol, 3.0 eq.) and HATU (1.781 g, 4.687 mmol, 1.5 eq.). The resulting mixture was stirred at room temperature for 16 h. TLC indicated the completion of reaction; the resulting reaction mixture was diluted with water (300 mL) and extracted by EtOAc (2×250 mL). The organic phase was dried over Na₂SO₄ and concentrated under vacuum to afford crude. The obtained crude material was purified by flash column chromatography using silica gel (230-400 mesh) as stationary phase (30% EtOAc in Hexane) to afford N—((R)-1-hydroxypropan-2-yl)-8-(4-(trifluoromethyl)cyclohex-1-en-1-yl)quinoline-3-carboxamide (Compound 7) (1.04 g, 2.74 mmol, 79%)

¹H NMR (DMSO-d₆, 400 MHz): δ ppm, 9.25 (d, J=2.4 Hz, 1H), 8.81 (d, J=2.4 Hz, 1H), 8.49 (d, J=8.0 Hz, 1H), 8.01-7.95 (m, 1H), 7.64-7.61 (m, 2H), 5.87 (br s, 1H), 4.81 (t, J=8.4 Hz, 1H), 4.10-4.05 (m, 1H), 3.54-3.48 (m, 1H), 3.42-3.28 (m, 1H) 2.89 (br s, 1H), 2.80-2.73 (m, 3H), 2.33-2.25 (m, 1H), 2.10-2.07 (m, 1H), 1.73-1.67 (m, 1H), 1.18 (d, J=6.8 Hz, 3H).

LCMS (Method A): 2.219 min, 98.50%, 242.0 nm, MS: ES+379.17 (M+1)

### Step-2

To a solution of N—((R)-1-hydroxypropan-2-yl)-8-(4-(trifluoromethyl)cyclohex-1-en-1-yl)quinoline-3-carboxamide (Compound 7) (0.9 g, 2.380 mmol, 1.0 eq.) and 1H-tetrazole (0.493 g, 7.135 mmol, 3.0 eq.) in DCM (25 mL) was added Di-tert-butyl N,N-Diisopropylphosphoramidite CAS: 137348-86-8 (1.95 g, 7.135 mmol, 3.0 eq.) and DMAP (0.145 g, 1.19 mmol, 0.5 eq.) at room temperature under nitrogen atmosphere. The resulting mixture was stirred at room temperature for 16 h. Then, H₂O₂ aq. (0.750 g, 7.135 mmol, 3.0 eq.) was added and reaction was stirred at room temperature for 2 h. TLC indicated the completion of reaction; the resulting reaction mixture was diluted with water (70 mL) and extracted with ETOAc (2×75 mL). Organic phase was dried over Na₂SO₄ and concentrated under vacuum to afford crude. The obtained crude material was purified by column chromatography using silica gel (60-100 mesh) as stationary phase (40% EtOAc in Hexane) to afford di-tert-butyl ((2R)-2-(8-(4-(trifluoromethyl)cyclohex-1-en-1-yl)quinoline-3-carboxamido)propyl)phosphate (A96) (0.230 g, 0.403 mmol, 16.10%)

¹H NMR (DMSO-d₆, 400 MHz): δ ppm, 9.25 (d, J=2.0 Hz, 1H), 8.81 (d, J=2.4 Hz, 1H), 8.72 ( ) (d, J=7.6 Hz, 1H), 8.01-7.99 (m, 1H), 7.66-7.62 (m, 2H), 5.87 (br s, 1H), 4.32-4.28 (m, 1H), 3.96-3.88 (m, 2H), 2.80-2.67 (m, 3H), 2.33-2.28 (m, 2H), 2.10-2.07 (m, 1H), 1.71-1.67 (m, 1H), 1.40 (s, 9H), 1.37 (s, 9H) Note: CF₃CH proton merged with DMSO solvent peak.

LCMS (Method A): 2.785 min, 100%, 210.0 nm, MS: ES+571.3 (M+1)

### Step-3

To a solution of di-tert-butyl ((2R)-2-(8-(4-(trifluoromethyl)cyclohex-1-en-1-yl)quinoline-3-carboxamido)propyl)phosphate (A96) (0.170 g, 0.298 mmol, 1.0 eq.) in DCM (5 mL) was added 4M HCl in Dioxane (1.5 mL) and stirred reaction mixture for 1 h. TLC indicated the completion of reaction; the reaction mixture was concentrated under reduced pressure to afford (2R)-2-(8-(4-(trifluoromethyl)cyclohex-1-en-1-yl)quinoline-3-carboxamido)propyl dihydrogen phosphate (Compound 105) (0.105 g, 0.215 mmol, Yield: 76.92%)

¹H NMR (DMSO-d₆, 400 MHz): δ ppm, 9.27 (d, J=2.0 Hz, 1H), 8.86 (d, J=1.6 Hz, 1H), 8.79 (d, J=7.6 Hz, 1H), 8.03-8.01 (m, 1H), 7.67-7.66 (m, 2H), 5.88 (br s, 1H), 4.29-4.23 (m, 1H), 3.96-3.84 (m, 2H), 2.80-2.67 (m, 4H), 2.33-2.25 (m, 1H), 2.10-2.07 (m, 1H), 1.74-1.68 (m, 1H), 1.23 (d, J=6.4 Hz, 3H). Note: Phosphate OH protons are exchanging in DMSO solvent.

¹H NMR (MeOD, 400 MHz): δ ppm, 9.61 (d, J=1.6 Hz, 1H), 9.45 (d, J=2.0 Hz, 1H), 8.35 (d, J=8.0 Hz, 1H), 8.12-8.10 (m, 1H), 8.05-8.01 (m, 1H), 6.08 (br s, 1H), 4.50-4.46 (m, 1H), 4.21-4.08 (m, 2H), 2.75-2.58 (m, 3H), 2.50-2.43 (m, 2H), 2.28-2.15 (m, 1H), 2.01-1.91 (m, 1H), 1.42 (d, J=6.4 Hz, 3H). Note: Phosphate OH and —CONH protons are exchanged in MeOD solvent.

HPLC (Method A): 3.649 min, 98.12%, 210.0 nm.

LCMS (Method A): 1.822 min, 100%, 254.0 nm, MS: ES+459.1 (M+1)

### Example 98—Chiral separation of N-((1H-pyrazol-5-yl)methyl)-8-(4-(trifluoromethyl)cyclohex-1-en-1-yl)quinoline-3-carboxamide (Compound 106 and Compound 107)

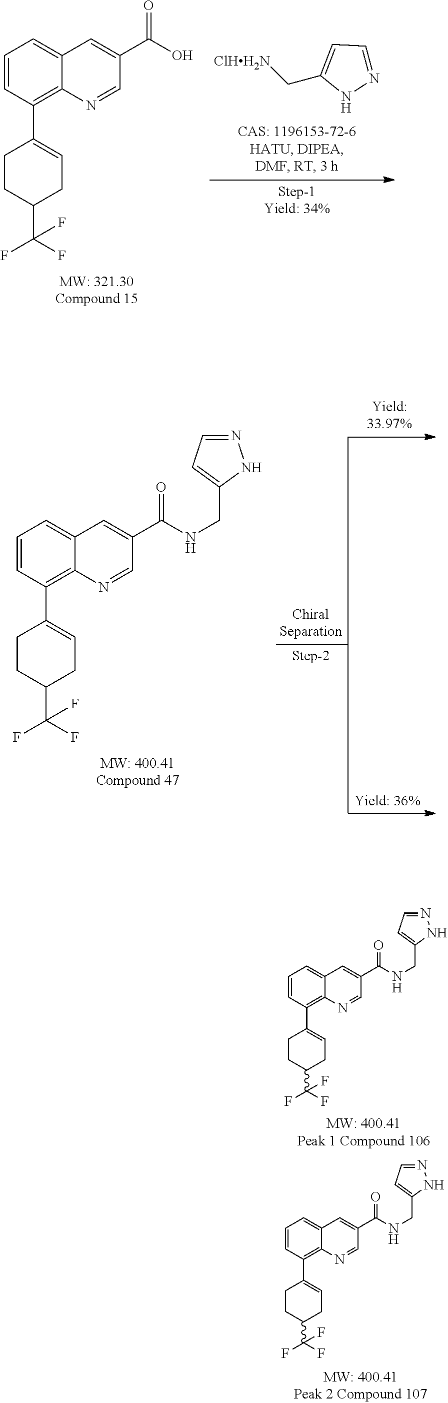

### Step-1

To a solution of 8-(4-(trifluoromethyl)cyclohex-1-en-1-yl)quinoline-3-carboxylic acid (Compound 15) (7.0 g, 21.786 mmol, 1.0 eq.), HATU (12.42 g, 32.710 mmol, 1.5 eq.) and DIPEA (11.25 mL, 65.420 mmol, 3.0 eq.) in DMF (70 mL) at 0° C. under nitrogen atmosphere stirred for 20 min, was added CAS: 1196153-72-6 (1H-Pyrazol-3-yl) methanamine hydrochloride (2.91 g, 21.786 mmol, 1.0 eq.) under Nitrogen. The resulting mixture was stirred for 3 h at room temperature. TLC indicated the completion of reaction; the resulting reaction mixture was quenched with ice-cold water (100 mL) and extracted with EtOAc (3×100 mL). The combined organic layers were dried over Na₂SO₄ and concentrated under reduced pressure to afford crude. The obtained crude material was purified by flash column chromatography using silica gel (230-400 mesh) as stationary phase (90% Ethyl acetate in hexane as gradient) to afford N-((1H-pyrazol-5-yl)methyl)-8-(4-(trifluoromethyl)cyclohex-1-en-1-yl)quinoline-3-carboxamide (Compound 47) (3.0 g, 7.492 mmol, Yield: 34%).

¹H NMR (DMSO-d₆, 400 MHz): δ ppm, 12.47 (s, 1H), 9.27-9.26 (m, 2H), 8.83 (d, J=2.4 Hz, 1H), 8.01-7.97 (m, 1H), 7.65-7.61 (m, 3H), 6.23 (s, 1H), 5.88 (br s, 1H), 4.54 (d, J=5.2 Hz, 2H), 3.40-3.28 (m, 1H), 2.80-2.67 (m, 3H), 2.51-1.88 (m, 2H), 1.74-1.64 (m, 1H).

LCMS (Method A): 2.10 min, 98.39%, 254.0 nm, MS: ES+401 (M+1)

HPLC (Method A): 7.44 min, 97.35%, 254.0 nm

Chiral HPLC: 4.57 min, 47.798%, 5.11 min, 48.72%, 245.0 nm

### Step-2

Chiral Separation of Compound 106 and Compound 107:

COLUMN ID: IG-REPACK, 250×30 mm 5 um

MOBILE PHASE A: 0.1% M NH₃ IN HEPTANE

MOBILE PHASE B: MDC: IPA: CAN (80:10:10)

FLOW RATE (ML/MIN): 40

INSTRUMENT ID: WELCH PREP SAIL 1000 UV Detector

METHOD: TIME: FLOW: % A: % B (INITIAL: 40:50:50), (45:40:50:40)

Input Quantity: 3.0 g.

Output Quantity: Compound 106=1.019 g (% Yield=33.97%) and Compound 107=1.081 g (% Yield=36%)

### Compound 106:

¹H NMR (DMSO-d6, 400 MHz): δ ppm, § 12.47 (s, 1H), 9.28-9.27 (m, 2H), 8.84 (d, J=2.0 Hz, 1H), 8.01-7.97 (m, 1H), 7.65-7.61 (m, 3H), 6.23 (br s, 1H), 5.87 (br s, 1H), 4.54 (br s, 2H), 2.81-2.51 (m, 3H), 2.28-2.07 (m, 3H), 1.74-1.64 (m, 1H).

LCMS (Method A): 2.219 min, 95.91%, 254.0 nm, MS: ES+401.3 (M+1)

HPLC (Method A): 7.38 min, 95.02%, 254.0 nm

Chiral HPLC (Method A): 4.74 min, 98.23%, 245 nm

### Compound 107:

¹H NMR (DMSO-d6, 400 MHz): δ ppm, § 12.47 (s, 1H), 9.28-9.25 (m, 2H), 8.84 (d, J=2.0 Hz, 1H), 8.01-7.98 (m, 1H), 7.65-7.61 (m, 3H), 6.23 (s, 1H), 5.87 (br s, 1H), 4.54 (d, J=5.2 Hz, 2H), 2.81-2.67 (m, 3H), 2.33-2.07 (m, 2H), 1.73-1.67 (m, 1H). Note: —CF₃CH proton merged in DMSO solvent peak.

LCMS (Method A): 2.218 min, 97.03%, 254.0 nm, MS: ES+401.27 (M+1)

HPLC (Method A): 7.39 min, 97.45%, 254.0 nm

Chiral HPLC (Method A): 5.35 min, 96.01%, 245 nm

### Example 99—Synthesis of 6-methoxy-N—((S)-1-(pyridin-2-yl)ethyl)-8-(4-(trifluoromethyl)cyclohex-1-en-1-yl)quinoline-3-carboxamide (Compound 108)

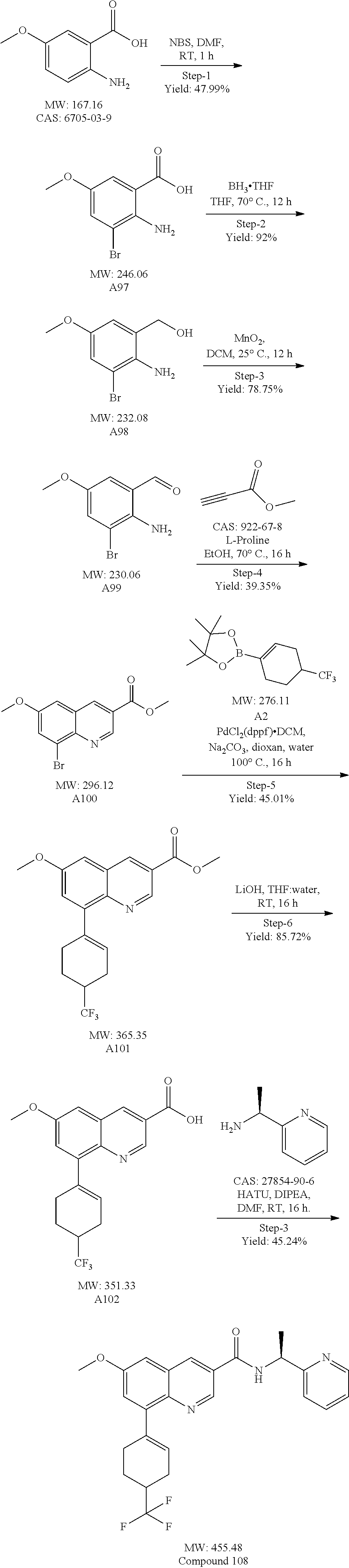

### Step-1

To a stirred solution of 5-Methoxy-2-aminobenzoic Acid (CAS: 6705-03-9) (1.0 g, 5.98 mmol, 1.0 eq.) in DMF (15.0 mL) was added NBS (1.28 g, 7.17 mmol, 1.2 eq.) at 0° C. The mixture was stirred at room temperature for 1 h. TLC indicated the completion of reaction; the reaction mixture was diluted with ice cold water (50.0 mL) and extracted by EtOAc (2×50 mL). The combined organic layer was dried over Na₂SO₄, filtered and concentrated under reduced pressure to obtained the crude which was purified by flash column chromatography on silica gel (100-200 mesh) (desired product eluted in 20% EtOAc in Hexane) to afford 2-amino-3-bromo-5-methoxybenzoic acid (A97) (0.708 g, 2.87 mmol, Yield: 47.99%).

¹H NMR (DMSO-d₆, 400 MHz): δ ppm, 7.38 (d, J=2.8 Hz, 1H), 7.33 (d, J=3.2 Hz, 1H), 3.69 (s, 3H).

Note: NH₂ and COOH protons might be exchange in DMSO solvent.

LCMS (Method A): 1.812 min, 100%, 254.0 nm, MS: ES+248.1 (M+2)

### Step-2

To a stirred solution of 2-amino-3-bromo-5-methoxybenzoic acid (A97) (0.7 g, 2.84 mmol, 1.0 eq.) in THF (5 mL) was added BH₃. THF (10.0 mL, 9.95 mmol, 3.5 eq., 1M in THF) at 0° C. The reaction mixture was stirred at 70° C. for 12 h. TLC indicated the completion of reaction; the reaction mixture was quenched with MeOH (50 mL) to obtain precipitate. The precipitate was filtered and filtrate was concentrated under reduced pressure to obtain crude material. The crude material was stirred with water (30 mL) and filtered. The residue was dissolved in DCM and dried over anhy. Na₂SO₄ and concentrated under reduced pressure to afford (2-bromo-6-(hydroxymethyl)-4-methoxyphenol (A98) (0.613 g, 2.64 mmol, 92.85% Yield) as a pale brown solid.

¹H NMR (DMSO-d₆, 400 MHz): δ ppm, 6.93 (d, J=2.8 Hz, 1H), 6.82 (d, J=2.8 Hz, 1H), 5.27 (t, J=5.6 Hz, 1H), 4.61 (br s, 2H), 4.40 (d, J=5.6 Hz, 2H), 3.66 (s, 3H).

LCMS (Method A): 1.505 min, 100%, 254.0 nm, MS: ES+234.0 (M+2)

### Step-3

To a stirred solution of (2-amino-3-bromo-5-methoxyphenyl) methanol (A98) (0.6 g, 2.58 mmol, 1 eq.) in DCM (10.0 mL) was added MnO₂ (2.28 g, 25.86 mmol, 10.0 eq.). The mixture was stirred at room temperature for 12 h. TLC indicated the completion of reaction; the reaction mixture was filtered on celite bed and filtrate was concentrated under reduced pressure to afford 2-amino-3-bromo-5-methoxybenzaldehyde (A99) (0.51 g, 2.21 mmol, Yield: 85.94%).

¹H NMR (DMSO-d₆, 400 MHz): δ ppm, 9.83 (s, 1H), 7.45 (d, J=2.8 Hz, 1H), 7.33 (d, J=2.8 Hz, 1H), 6.71 (br s, 2H), 3.74 (s, 3H); LCMS (Method A): 2.037 min, 98.79%, 254.0 nm, MS: ES+232 (M+2)

### Step-4

A stirred solution of 2-amino-3-bromo-5-methoxybenzaldehyde (A99) (0.50 g, 2.17 mmol, 1.0 eq.) in EtOH (10.0 mL) was prepared. To this solution were added L-proline (0.124 g, 1.08 mmol, 0.5 eq.) and CAS: 922-67-8 (0.237 g, 2.82 mmol, 1.3 eq.) at RT and resulting reaction mixture was stirred at 70° C. for 16 h. TLC indicated the completion of reaction; the reaction mixture was concentrated under reduced pressure to obtain crude material. The crude material was purified by column chromatography (desired product eluted in 30% Ethyl acetate in hexane) to afford methyl 8-bromo-6-methoxyquinoline-3-carboxylate (A100) (0.253 g, 0.854 mmol, 39.35% Yield).

¹H NMR (DMSO-d₆, 400 MHz): δ ppm, 9.21 (d, J=1.6 Hz, 1H), 8.93 (s, 1H), 7.97 (d, J=2.4 Hz, 1H), 7.71 (d, J=2.0 Hz, 1H), 3.96 (s, 3H), 3.92 (s, 3H).

LCMS (Method A): 2.215 min, 100%, 254.0 nm, MS: ES+298.0 (M+2)

### Step-5

To a stirred solution of methyl 8-bromo-6-methoxyquinoline-3-carboxylate (A100) (1.00 g, 3.37 mmol, 1.0 eq.) in 1,4-dioxane:water (4:1, 50 mL) were added 4, 4, 5,5-tetramethyl-2-(4-(trifluoromethyl)cyclohex-1-en-1-yl)-1, 3,2-dioxaborolane (A2) (1.39 g, 5.067 mmol, 1.5 eq.), and Cs₂CO₃ (3.29 g, 10.134 mmol, 3.0 eq.) at room temperature. The reaction mixture was stirred and degassed with N₂ gas for 15 min. After then PdCl₂(dppf)·DCM (0.413 g, 0.506 mmol, 0.15 eq.) was added at room temperature under N₂ atmosphere. The resulting mixture was stirred at 100° C. for 16 h. TLC indicated the completion of reaction; the resulting reaction mixture was filtered through celite bed and concentrated under reduced pressure to afford crude. The crude was purified by column chromatography using silica gel (230-400 mesh) as stationary phase (32% EtOAc in Hexane) to afford methyl (A101)methyl 6-methoxy-8-(4-(trifluoromethyl)cyclohex-1-en-1-yl)quinoline-3-carboxylate (1.25 g, 3.42 mmol, Yield: 45.01%).

¹H NMR (DMSO-d₆, 400 MHz): δ ppm, 9.12 (d, J=2.4 Hz, 1H), 8.87 (d, J=2.4 Hz, 1H), 7.55 (d, J=2.8 Hz, 1H), 7.29 (d, J=2.8 Hz, 1H), 5.88 (br s, 1H), 3.94 (s, 3H), 3.90 (s, 3H), 2.81-2.74 (m, 1H), 2.67-2.63 (m, 2H), 2.50-2.43 (m, 1H), 2.29-2.22 (m, 1H), 2.08-2.05 (m, 1H), 1.72-1.62 (m, 1H).

LCMS (Method A): 2.923 min, 93.95%, 254.0 nm, MS: ES+366.3 (M+1).

### Step-6

A solution of methyl 6-methoxy-8-(4-(trifluoromethyl)cyclohex-1-en-1-yl)quinoline-3-carboxylate (A101) (1.25 g, 3.42 mmol, 1.0 eq.) and LiOH·H₂O (0.410 g, 10.27 mmol, 3.0 eq.) in MeOH:H₂O (20 mL, 7:3) was prepared at 0° C. under nitrogen atmosphere. The resulting mixture was stirred at 100° C. for 16 h. TLC indicated the completion of reaction; the resulting reaction mixture was concentrated under reduced pressure and obtained residue was poured onto 10% citric acid solution, then filtered off to afford the desired product 6-methoxy-8-(4-(trifluoromethyl)cyclohex-1-en-1-yl)quinoline-3-carboxylic acid (A102) (1.03 g, 2.93 mmol, Yield: 85.72%).

¹H NMR (DMSO-d₆, 400 MHz): δ ppm, 9.12 (d, J=2.0 Hz, 1H), 8.79 (d, J=2.0 Hz, 1H), 7.50 (d, J=2.8 Hz, 1H), 7.25 (d, J=2.8 Hz, 1H), 5.87 (br s, 1H), 3.90 (s, 3H), 2.78-2.75 (m, 1H), 2.68-2.65 (m, 2H), 2.50-2.43 (m, 1H), 2.29-2.22 (m, 1H), 2.07-2.05 (m, 1H), 1.70-1.64 (m, 1H). Note: Acid (COOH) proton exchanged with deuterium

LCMS (Method A): 2.512 min, 94.61%, 254.0 nm, MS: ES+352.3 (M+1)

### Step 7

To a stirred solution of 6-methoxy-8-(4-(trifluoromethyl)cyclohex-1-en-1-yl)quinoline-3-carboxylic acid (A102) (0.900 g, 2.560 mmol, 1.0 eq.) in DMF (10 mL), was added DIPEA (0.998 g, 7.680 mmol, 3.0 eq.) and HATU (1.45 g, 3.840 mmol, 1.5 eq.) at 0° C. The reaction mixture was stirred under the N₂ atmosphere for 10 minutes. After 10 min of stirring at 0° C. (S)-1-(237yridine-2-yl)ethan-1-amine (CAS: 27854-90-6) (0.312 mg, 2.56 mmol, 1.0 eq.) was added and the resulting reaction mixture was stirred at RT for 16 h. TLC indicated the completion of reaction; the resulting reaction mixture was quenched with ice-cold water (100 mL) and extracted with ethyl acetate (125 mL×2). The combined organic layers were dried over Na₂SO₄, filtered and concentrated under reduced pressure to afford crude. The crude was purified by column chromatography using silica gel (230-400 mesh) as stationary phase (32% EtOAc in Hexane) to afford 6-methoxy-N—((S)-1-(237yridine-2-yl)ethyl)-8-(4-(trifluoromethyl)cyclohex-1-en-1-yl)quinoline-3-carboxamide (Compound 108) (0.527 g, 1.15 mmol, 45.24%).

¹H NMR (DMSO-d₆, 400 MHz): δ ppm, 9.17-9.12 (m, 2H), 8.75 (d, J=2.0 Hz, 1H), 8.55 (d, J=4.0 Hz, 1H), 7.78 (d, J=6.8 Hz, 1H), 7.48-7.46 (d, J=8.0 Hz, 1H), 7.40 (d, J=2.4 Hz, 1H), 7.29-7.25 (m, 2H), 5.89 (br s, 1H), 5.29-5.23 (m, 1H), 3.91 (s, 3H), 2.89-2.67 (m, 3H), 2.50-2.44 (m, 1H), 2.32-2.23 (m, 1H), 2.08-2.05 (m, 1H), 1.72-1.66 (m, 1H), 1.64 (d, J=6.8 Hz, 3H).

HPLC (Method A): 9.24 min, 95.12%, 210.0 nm.

LCMS (Method A): 9.915 min, 96.52%, 254.0 nm, MS: ES+456.5 (M+1).

### Example 100—Synthesis of 6-methoxy-N—((R)-1-(oxazol-2-yl)ethyl)-8-(4-(trifluoromethyl)cyclohex-1-en-1-yl)quinoline-3-carboxamide (Compound 109)

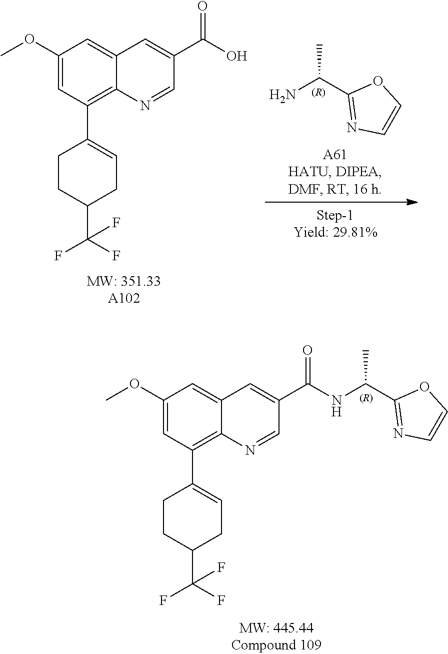

### Step-1

To a stirred solution of 6-methoxy-8-(4-(trifluoromethyl)cyclohex-1-en-1-yl)quinoline-3-carboxylic acid (A102) (0.75 g, 2.13 mmol, 1.0 eq.) in DMF (10 mL) was added DIPEA (0.82 g, 6.40 mmol, 3.0 eq.) and HATU (1.21 g, 3.20 mmol, 1.5 eq.) at 0° C. Followed by addition of (R)-1-(oxazol-2-yl)ethan-1-amine (A61) (0.238 g, 2.13 mmol, 1.0 eq.) and the resulting reaction mixture was stirred at room temperature for 16 h. TLC indicated the completion of reaction; the resulting reaction mixture was quenched with ice-cold water (100 mL) and extracted with ethyl acetate (125 mL×2). The combined organic layers were dried over Na₂SO₄, filtered and concentrated under reduced pressure to afford crude. The crude was purified by column chromatography using silica gel (230-400 mesh) as stationary phase (40% EtOAc in Hexane) to afford 6-methoxy-N—((R)-1-(oxazol-2-yl)ethyl)-8-(4-(trifluoromethyl)cyclohex-1-en-1-yl)quinoline-3-carboxamide (Compound 109) (0.264 g, 0.593 mmol, 29.81%).

¹H NMR (DMSO-d₆, 400 MHz): δ ppm, 9.32 (d, J=8.0 Hz, 1H), 9.10 (d, J=1.6 Hz, 1H), 8.73 (d, J=1.6 Hz, 1H), 8.08 (s, 1H), 7.39 (d, J=2.8 Hz, 1H), 7.25 (d, J=2.8 Hz, 1H), 7.18 (s, 1H), 5.89 (br s, 1H), 5.40-5.33 (m, 1H), 3.90 (s, 3H), 3.78-2.66 (m, 3H), 2.47-2.44 (m, 1H), 2.30-2.26 (m, 1H), 2.23-2.08 (m, 1H), 1.75-1.61 (m, 1H), 1.60 (d, J=6.8 Hz, 3H).

LCMS (Method A): 2.46 min, 98.31%, 254.0 nm, MS: ES+446.3 (M+1).

HPLC (Method A): 8.77 min, 99.62%, 254.0 nm.

Chiral HPLC: Peak-1: 3.11 min, 48.01%, 248.0 nm; Peak-2: 3.83 min, 48.04%, 248.0 nm

### Example 101—Synthesis of N—((R)-1-hydroxypropan-2-yl)-6-methoxy-8-(4-(trifluoromethyl)cyclohex-1-en-1-yl)quinoline-3-carboxamide (Compound 110)

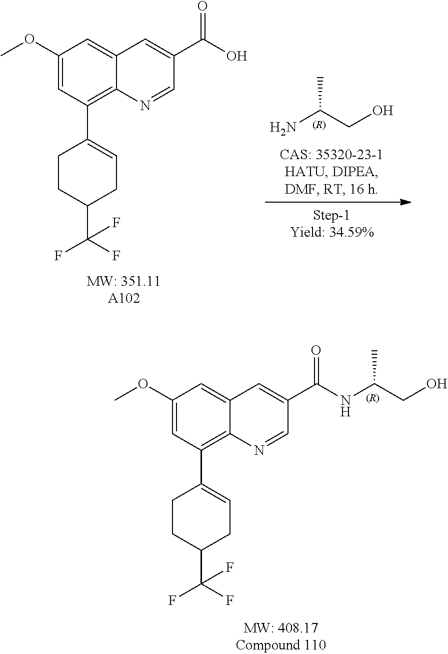

### Step-1

To a stirred solution of 6-methoxy-8-(4-(trifluoromethyl)cyclohex-1-en-1-yl)quinoline-3-carboxylic acid (A102) (0.750 g, 2.13 mmol, 1.0 eq.) in DMF (10 mL) was added DIPEA (0.82 g, 6.40 mmol, 3.0 eq.) and HATU (1.21 g, 3.20 mmol, 1.5 eq.) at 0° C. The reaction mixture was stirred under the N₂ atmosphere. After 15 min of stirring at 0° C. (R)-2-aminopropan-1-ol (CAS: 35320-23-1) (0.159 g, 2.13 mmol, 1.0 eq.), was added at same temperature and the resulting reaction mixture was stirred at 0° C. to room temperature for 16 h. TLC indicated the completion of reaction; the resulting reaction mixture was quenched with ice-cold water (100 mL) and extracted with ethyl acetate (125 mL×2). The combined organic layers were dried over Na₂SO₄, filtered and concentrated under reduced pressure to afford crude. The crude was purified by column chromatography using silica gel (230-400 mesh) as stationary phase (60% EtOAc in Hexane) to afford N—((R)-1-hydroxypropan-2-yl)-6-methoxy-8-(4-(trifluoromethyl)cyclohex-1-en-1-yl)quinoline-3-carboxamide (Compound 110) (0.281 g, 0.688 mmol, 34.59%)

¹H NMR (DMSO-d₆, 400 MHz): δ ppm, 9.07 (d, J=1.6 Hz, 1H), 8.67 (d, J=1.6 Hz, 1H), 8.42 (d, J=8.0 Hz, 1H), 7.38 (d, J=2.8 Hz, 1H), 7.24 (d, J=2.4 Hz, 1H), 5.89 (br s, 1H), 4.78 (t, J=6.0 Hz, 1H), 4.10-4.03 (m, 1H), 3.96 (s, 3H), 3.53-3.47 (m, 2H), 2.77-2.67 (m, 3H), 2.33-2.23 (m, 1H), 2.08-2.05 (m, 1H), 1.72-1.65 (m, 1H), 1.17 (d, J=6.8 Hz, 3H). Note: CF₃—CH proton merged with DMSO solvent peak which is clearly observed in D₂O spectrum.

LCMS (Method A): 2.25 min, 99.03%, 254.0 nm, MS: ES+409.4 (M+1)

HPLC (Method A): 8.02 min, 98.79%, 254.0 nm.

Chiral HPLC: Peak-1: 3.64 min, 45.42%, 246.0 nm. Peak-2: 3.95 min, 52.76%, 246.0 nm.

### Example 102—Synthesis of 6-methoxy-N-(oxazol-2-ylmethyl)-8-(4-(trifluoro methyl)cyclohex-1-en-1-yl)quinoline-3-carboxamide (Compound 111)

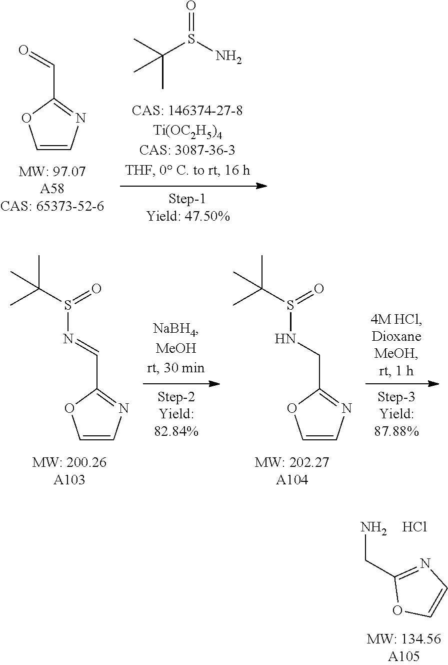

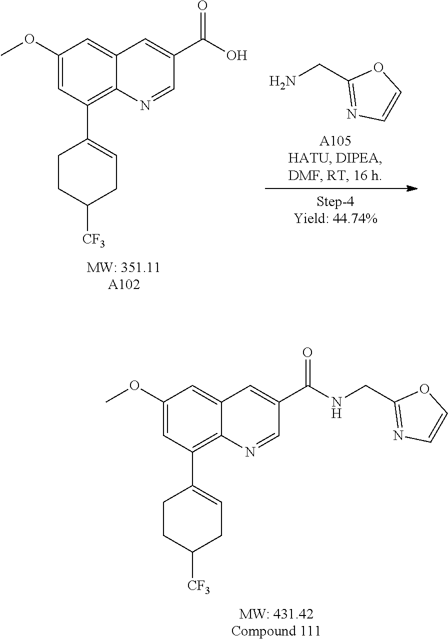 [SMILES: COc1cc(C2=CCC(C(F)(F)F)CC2)c2ncc(C(=O)O)cc2c1 ; NCc1ncco1 ; COc1cc(C2=CCC(C(F)(F)F)CC2)c2ncc(C(=O)NCc3ncco3)cc2c1]

### Step-1

To a stirred solution of oxazole-2-carbaldehyde (A58) (5.0 g, 51.5 mmol, 1.0 eq) in DMF (10 mL) was added 2-Methylpropane-2-sulfinamide CAS: 146374-27-8 (7.4 g, 61.8 mmol, 1.2 eq), and Piperidine-1-carbaldehyde CAS: 3087-36-3 (25.5 ml, 10.3 mmol, 2.0 eq) at 0° C. under Nitrogen atmosphere. Then reaction mixture was stirred at RT for 16 h. TLC (50% EtOAc in Hexane as a mobile phase) indicated the completion of reaction. The resulting reaction mixture was poured onto water (100 mL) and extracted with EtOAc (3×200 mL). The combined organic layer was dried over Na₂SO₄, filtered and concentrated under reduced pressure to obtained the crude. The resulting crude product was purified by column chromatography using silica gel 230-400 mesh size and desired product eluted in 40% EtOAc in hexane to afford (E)-2-methyl-N-(oxazol-2-ylmethylene) propane-2-sulfinamide (A103) (4.9 g, 24.46 mmol, Yield: 47.50%).

¹H NMR (DMSO, 400 MHz): δ ppm 8.45 (s, 1H), 8.28 (s, 1H), 7.62 (s, 1H), 1.27 (s, 9H).

LCMS (Method A): LCMS does not support the desired product mass.

### Step-2

To a stirred solution of (E)-2-methyl-N-(oxazol-2-ylmethylene) propane-2-sulfinamide (A103) (4.9 g, 29.9 mmol, 1.0 eq) in MeOH (20 mL) was added NaBH₄ (1.7 g, 44.9 mmol, 2.0 eq) at 0° C. The reaction mixture was stirred at RT for 30 min and monitored on TLC (5% DCM: MeOH as mobile phase). The resulting reaction mixture was poured into water (400 mL) and extracted with EtOAc (3×200 mL). The combined organic layer was dried over Na₂SO₄, filtered and concentrated under reduced pressure to obtained the crude. The resulting crude product was purified by column chromatography (desired product eluted in 2% MeOH in DCM) to afford 2-methyl-N-(oxazol-2-ylmethyl) propane-2-sulfinamide (A104) (4.1 g, 20.26 mmol, Yield: 82.84%).

LCMS (Method A): 1.303 min, 100.0%, 210.0 nm MS: ES+203.0 (M+1).

¹H NMR (DMSO, 400 MHz): δ ppm 7.64 (s, 1H), 7.09 (s, 1H), 4.49-4.37 (m, 2H), 3.94 (t, J=8.8 Hz 1H), 1.24 (s, 9H).

### Step-3

To a stirred solution of 2-methyl-N-(oxazol-2-ylmethyl) propane-2-sulfinamide (A104) (9.0 g, 44.4 mmol, 1.0 eq) in MeOH (90 mL) was added 4M HCl in Dioxane (11.68 g, 31.1 mmol, 7.0 v) at 0° C. and reaction mixture was stirred at RT for 1 h. The reaction was monitored on TLC (using; 5% DCM:MeOH as mobile phase). After completion, the resulting reaction mixture was directly concentrated under reduced pressure to obtained the crude material which was triturated using pentane to afford oxazol-2-ylmethanamine hydrochloride (A105) (7.0 g, 52.02 mmol, Yield: 87.88%).

LCMS (Method A): LCMS does not support the desired product mass

¹H NMR (DMSO, 400 MHz): δ ppm 8.94 (s, 3H), 8.23 (s, 1H), 7.30 (s, 1H), 4.22 (s, 2H).

### Step-4

To a stirred solution of 6-methoxy-8-(4-(trifluoromethyl)cyclohex-1-en-1-yl)quinoline-3-carboxylic acid (A102) (0.35 g, 0.996 mmol, 1.0 eq.) in DMF (15 mL) were added DIPEA (0.38 g, 2.99 mmol, 3.0 eq.) and HATU (0.568 g, 1.49 mmol, 1.5 eq.) at 0° C. The reaction mixture was stirred under the N₂ atmosphere. After 15 min of stirring at 0° C. Oxazol-2-ylmethanamine (A105) (0.097 g, 0.996 mmol, 1.0 eq.) was added at same temperature and the resulting reaction mixture was stirred at 0° C. to room temperature for 16 h. TLC indicated the completion of reaction; the resulting reaction mixture was quenched with ice-cold water (50 mL) and extracted with ethyl acetate (100 mL×2). The combined organic layers were dried over Na₂SO₄, filtered and concentrated under reduced pressure to afford crude. The crude was purified by column chromatography using silica gel (230-400 mesh) as stationary phase (55% EtOAc in Hexane) to afford 6-methoxy-N-(oxazol-2-ylmethyl)-8-(4-(trifluoromethyl)cyclohex-1-en-1-yl)quinoline-3-carboxamide (Compound 111) (0.165 g, 0.382 mmol, 44.74%).

¹H NMR (DMSO-d₆, 400 MHz): δ ppm, 9.48 (t, J=5.6 Hz, 1H), 9.00 (d, J=2.0 Hz, 1H), 8.72 (d, J=2.0 Hz, 1H), 8.09 (s, 1H), 7.40 (d, J=2.4 Hz, 1H), 7.26 (d, J=2.8 Hz, 1H), 7.19 (s, 1H), 5.89 (br s, 1H), 4.66 (d, J=5.6 Hz, 2H), 3.91 (s, 3H), 2.79-2.67 (m, 3H), 2.33-2.23 (m, 2H), 2.09-2.06 (m, 1H), 1.72-1.63 (m, 1H).

LCMS (Method C): 2.413 min, 96.37%, 254.0 nm, MS: ES+432.3 (M+1)

HPLC (Method B): 6.93 min, 95.22%, 210.0 nm.

Chiral HPLC: Peak-1: 4.81 min, 47.98%, 246.0 nm; Peak-2: 5.33 min, 49.82%, 246.0 nm.

### Example 103—Synthesis of N-((1H-pyrazol-3-yl)methyl)-6-methoxy-8-(4-(trifluoro methyl)cyclohex-1-en-1-yl)quinoline-3-carboxamide (Compound 112)

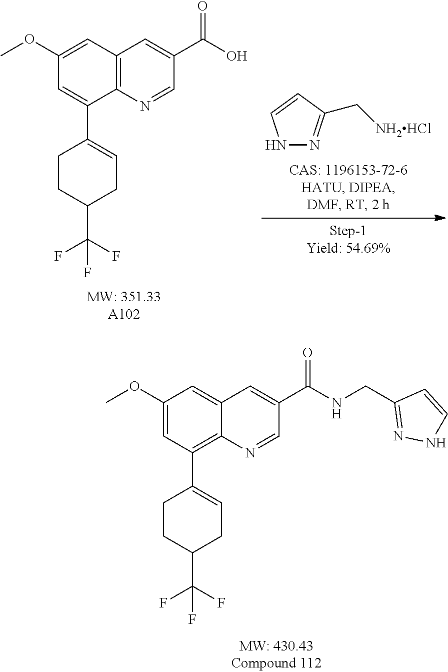

### Step-1

To a stirred solution of 6-methoxy-8-(4-(trifluoromethyl)cyclohex-1-en-1-yl)quinoline-3-carboxylic acid (A102) (0.1 g, 0.28 mmol, 1.0 eq.), in DMF (2.0 mL) was added HATU (0.123 g, 0.56 mmol, 2.0 eq.) and DIPEA (0.110 g, 0.85 mmol, 3.0 eq.) at 0° C. Followed by addition of (1H-pyrazol-3-yl) methanamine hydrochloride (CAS: No: 1196153-72-6) (0.056 g, 0.42 mmol, 1.5 eq.) and the resulting reaction mixture was stirred at room temperature for 2 h. TLC indicated the completion of reaction; the resulting reaction mixture was quenched with ice-cold water (20 mL) and extracted with ethyl acetate (50 mL×3). The combined organic layers were dried over Na₂SO₄, filtered and concentrated under reduced pressure to afford crude. The crude was purified by column chromatography using silica (230-400 Mesh) as stationary phase (45-50% Ethyl Acetate in Hexane) to afford N-((1H-pyrazol-3-yl)methyl)-6-methoxy-8-(4-(trifluoromethyl)cyclohex-1-en-1-yl)quinoline-3-carboxamide (Compound 112) (0.067 g, 0.155 mmol, Yield: 54.69%).

¹H NMR (DMSO-d₆, 400 MHz): δ ppm, 12.63 (br s, 1H), 9.20 (br s, 1H), 9.10 (d, J=2.0 Hz, 1H), 8.70 (d, J=2.0 Hz, 1H), 7.66 (br s, 1H), 7.38 (d, J=2.8 Hz, 1H), 7.24 (d, J=2.8 Hz, 1H), 6.22 (s, 1H), 5.88 (s, 1H), 4.53 (br s, 2H), 3.93 (s, 3H), 2.78-2.67 (m, 3H), 2.33-2.23 (m, 1H), 2.08-2.05 (m, 1H), 1.72-1.62 (m, 1H). Note: CF₃—CH proton merged with DMSO solvent peak which is clearly observed in MeOD.

¹H NMR (MeOD, 400 MHz): δ ppm, 9.09 (d, J=2.0 Hz, 1H), 8.67 (d, J=2.4 Hz, 1H), 7.61 (br s, 1H), 7.30 (d, J=1.2 Hz, 2H), 6.37 (s, 1H), 5.86 (s, 1H), 4.70 (s, 2H), 4.01 (s, 3H), 2.74-2.71 (m, 3H), 2.65-2.49 (m, 1H), 2.39-2.31 (m, 1H), 2.16-2.13 (m, 1H), 1.91-1.80 (m, 1H). Note: —CONH and —NH protons exchanged in MeOD NMR.

LCMS (Method A): 2.30 min, 98.42%, 254 nm, MS: ES+431.7 (M+1)

HPLC (Method A): 8.07 min, 98.25%, 254 nm.

### Example 104—Synthesis of N—((S)-1-hydroxy-3-methoxypropan-2-yl)-6-methoxy-8-(4-(trifluoromethyl)cyclohex-1-en-1-yl)quinoline-3-carboxamide (Compound 113)

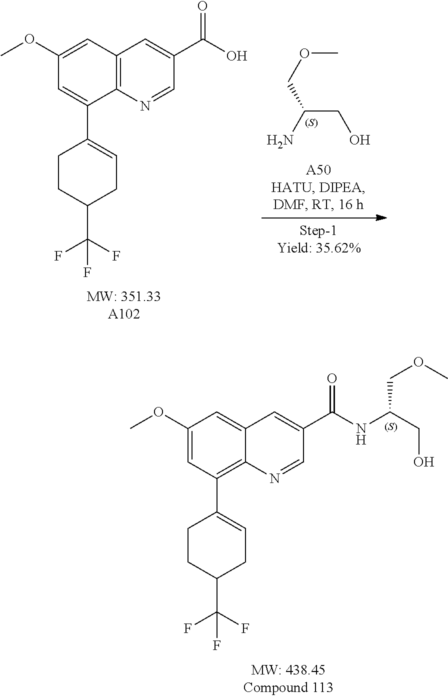

### Step-1

To a solution 6-methoxy-8-(4-(trifluoromethyl)cyclohex-1-en-1-yl)quinoline-3-carboxylic acid (A102) (0.09 g, 0.256 mmol, 1.0 eq.) in DMF (1 mL), was added HATU (0.14 g, 0.38 mmol, 1.5 eq.) and DIPEA (0.13 mL, 0.76 mmol, 3.0 eq.) at 0° C. under the N₂ atmosphere. After 10 min of stirring at 0° C., (S)-2-amino-3-methoxypropan-1-ol (A50) (0.053 g, 0.51 mmol, 2.0 eq.) was added and the resulting reaction mixture was stirred at 0° C. to room temperature for 16 h. TLC indicated the completion of reaction; the resulting reaction mixture was quenched with ice-cold water (10 mL) and extracted with ethyl acetate (20 mL×3). The combined organic layers were dried over Na₂SO₄, filtered and concentrated under reduced pressure to afford crude. The crude was purified by column chromatography using silica gel (230-400 Mesh) as stationary phase (70% EA in Hexane) to afford N—((S)-1-hydroxy-3-methoxypropan-2-yl)-6-methoxy-8-(4-(trifluoromethyl)cyclohex-1-en-1-yl)quinoline-3-carboxamide (Compound 113) (0.04 g, 0.091 mmol, Yield: 35.62%).

¹H NMR (MeOD, 400 MHz): δ ppm, 9.07 (d, J=2.0 Hz, 1H), 8.66 (d, J=2.0 Hz, 1H), 7.30 (dd, J=2.8, 5.2 Hz, 2H), 5.87 (br s, 1H), 4.39-3.36 (m, 1H), 3.97 (s, 3H), 3.78 (dd, J=1.2, 5.6 Hz, 2H), 3.68-3.62 (m, 2H), 3.42 (s, 3H), 2.75-2.62 (m, 3H), 2.54-2.50 (m, 1H), 2.39-2.36 (m, 1H), 2.16-2.14 (m, 1H), 1.88-1.84 (m, 1H). Note: —CONH and —OH protons exchanged in MeOD NMR.

¹H NMR (DMSO-d₆, 400 MHz): δ ppm, 9.08 (d, J=2.0 Hz, 1H), 8.69 (d, J=2.0 Hz, 1H), 8.47 (d, J=8.4 Hz, 1H), 7.38 (d, J=2.8 Hz, 1H), 7.24 (d, J=2.8 Hz, 1H), 5.89 (m, 1H), 4.82 (t, J=5.6 Hz, 1H), 4.21-4.16 (m, 1H), 3.91 (s, 3H), 3.54-3.47 (m, 4H), 3.25 (s, 3H), 2.78-2.67 (m, 3H), 2.46-2.44 (m, 1H), 2.33-2.23 (m, 1H), 2.08-2.05 (m, 1H), 1.72-1.62 (m, 1H).

LCMS (Method A): 2.270 min, 99.48%, 254.0 nm, MS: ES+439.6 (M+1).

HPLC (Method A): 6.89 min, 95.15%, 254.0 nm.

CHIRAL HPLC (Method A): Peak-1: 20.361 min, 48.11%, 246.0 nm; Peak-2: 24.125 min, 48.83%, 246.0 nm.

### Example 105—Synthesis of 6-methoxy-N-(3-(methylsulfonamido)propyl)-8-(4-(trifluoromethyl)cyclohex-1-en-1-yl)quinoline-3-carboxamide (Compound 114)

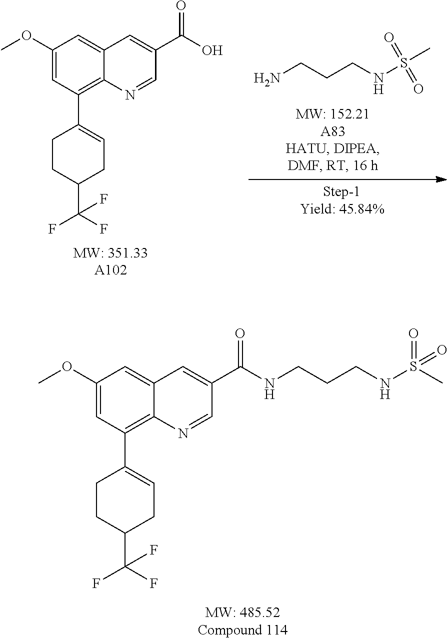

### Step-1

To a stirred solution of 6-methoxy-8-(4-(trifluoromethyl)cyclohex-1-en-1-yl)quinoline-3-carboxylic acid (A102) (0.050 g, 0.142 mmol, 1.0 eq.) in DMF (5 mL), was added N-(3-aminopropyl) methanesulfonamide (A83) (0.026 mg, 0.142 mmol, 1.0 eq.), TEA (0.043 g, 0.426 mmol, 3.0 eq.) and HATU (0.080 g, 0.213 mmol, 1.5 eq.). The resulting reaction mixture was stirred at room temperature for 16 h. TLC indicated the completion of reaction; the resulting reaction mixture was diluted with water (100 mL) and extracted by EtOAc (2×125 mL). The organic phase was dried over Na₂SO₄ and concentrated under vacuum to afford crude. The obtained crude material was purified by flash column chromatography using silica gel (230-400 mesh) as stationary phase (10% MeOH in DCM) to afford 6-methoxy-N-(3-(methylsulfonamido)propyl)-8-(4-(trifluoromethyl)cyclohex-1-en-1-yl)quinoline-3-carboxamide (Compound 114) (0.029 g, 0.060 mmol, 45.84%)

¹H NMR (MeOD, 400 MHz): δ ppm, 9.06 (d, J=2.0 Hz, 1H), 8.82 (br s, 1H), 8.65 (d, J=2.0 Hz, 1H), 7.31 (d, J=2.4 Hz, 1H), 5.86 (br s, 1H), 4.02 (s, 3H), 3.60-3.56 (m, 2H), 3.21 (t, J=6.4 Hz, 2H), 2.98 (s, 3H), 2.75-2.62 (m, 3H), 2.54-2.50 (m, 1H), 2.9-2.36 (m, 1H), 2.17-2.14 (m, 1H), 1.96-1.87 (m, 3H). Note: —CONH and MeSO₂NH-proton exchanged in MeOD NMR.

¹H NMR (DMOS-d₆, 400 MHz): δ ppm, 9.06 (d, J=2.0 Hz, 1H), 8.76 (t, J=5.6 Hz, 1H), 8.66 (d, J=2.0 Hz, 1H), 7.39 (d, J=2.4 Hz, 2H), 7.25 (d, J=2.8 Hz, 2H), 7.04 (t, J=5.6 Hz, 2H), 5.86 (br s, 1H), 3.92 (s, 3H), 3.06-3.01 (m, 2H), 2.91 (s, 3H), 2.88-2.73 (m, 4H), 2.33-2.23 (m, 1H), 2.08-2.06 (m, 1H), 1.80-1.65 (m, 2H).

HPLC (Method B): 7.18 min, 96.09%, 254.0 nm,

LCMS (Method A): 2.357 min, 97.41%, 254.0 nm, MS: ES+486.89

### Example 106—Synthesis of N-isopropyl-6-methoxy-8-(4-(trifluoromethyl)cyclohex-1-en-1-yl)quinoline-3-carboxamide (Compound 115)

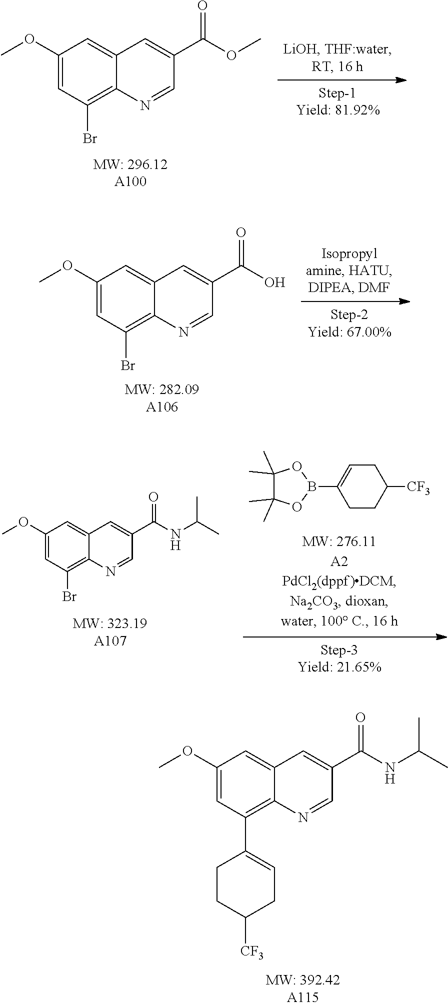

### Step-1

To the stirred solution of methyl 8-bromo-6-methoxyquinoline-3-carboxylate (A100) (0.2 g, 0.675 mmol, 1.0 eq.) in MeOH:H₂O (5.0 mL, 7:3) was added LiOH (0.270 g, 6.75 mmol, 10.0 eq.) and reaction mixture was stirred for 16 h at room temperature. TLC indicated the completion of reaction; the resulting reaction mixture was directly concentrated under reduced pressure to obtained crude product. The crude product was neutralized by using aq. 10% citric acid solution to obtained precipitate. The precipitate was collected by filtration and dried under reduce pressure to afford 8-bromo-6-methoxyquinoline-3-carboxylic acid (A106) (0.156 g, 0.553 mmol, Yield: 81.92%).

¹H NMR (DMSO-d₆, 400 MHz): δ ppm, 13.38 (br s, 1H), 9.21 (d, J=1.6 Hz, 1H), 8.89 (d, J=1.6 Hz, 1H), 7.95 (d, J=2.4 Hz, 1H), 7.68 (d, J=2.4 Hz, 1H), 3.92 (s, 3H).

LCMS (Method A): 1.851 min, 100%, 254.0 nm, MS: ES+284.0 (M+2)

### Step-2

A stirred solution of 8-bromo-6-methoxyquinoline-3-carboxylic acid (A106) (0.15 g, 0.531 mmol, 1.0 eq.) in DMF (2.5 mL) was prepared. To this solution, isopropyl amine CAS: 75-31-0 (0.03 g, 0.531 mmol, 1.0 eq.), DIPEA (0.20 g, 1.593 mmol, 3.0 eq.) and HATU (0.30 g, 0.796 mmol, 1.5 eq.) was added and resulting mixture was stirred at room temperature for 16 h. TLC indicated the completion of reaction; the resulting reaction mixture was diluted with water (75 mL) and again stirred at RT for another 1 h to obtained solid material. The obtained solid material was filtered on Buchner funnel and dried under high vacuum to afford 8-bromo-N-isopropyl-6-methoxyquinoline-3-carboxamide (A107) (0.11 g, 0.340 mmol, 67%).

¹H NMR (DMSO-₆, 400 MHz): δ ppm, 9.15 (d, J=2.0 Hz, 1H), 8.72 (d, J=1.6 Hz, 1H), 8.62 (d, J=7.2 Hz, 1H), 7.92 (d, J=2.4 Hz, 1H), 7.54 (d, J=2.8 Hz, 1H), 4.18-4.13 (m, 1H), 3.93 (s, 3H), 1.22 (d, J=6.8 Hz, 6H).

LCMS (Method A): 1.946 min, 99.51%, 254.0 nm, MS: ES+325.1 (M+2)

### Step-3

A stirred solution of 4, 4, 5,5-tetramethyl-2-(4-(trifluoromethyl)cyclohex-1-en-1-yl)-1, 3,2-dioxaborolane (A2) (0.084 g, 0.306 mmol, 1.1 eq.), 8-bromo-N-isopropyl-6-methoxyquinoline-3-carboxamide (A107) (0.09 g, 0.278 mmol, 1.0 eq.) and Na₂CO₃ (0.058 g, 0.556 mmol, 2.0 eq.) in Dioxane (4.0 mL) and water (1.0 mL) at RT was prepared. The resulting reaction mixture was purged under Nitrogen atmosphere for 10-15 min. Then added Pd(dppf)Cl₂·DCM (0.004 g, 0.042 mmol, 0.15 eq.) and stirred at 100° C. for 16 h. TLC indicated the completion of reaction; the resulting reaction mixture was filtered through celite bed and filtrate was concentrated under reduced pressure to obtain crude product. The obtained crude product was purified by flash column chromatography (desired product eluted in 35% EtOAc in Hexane) to afford N-isopropyl-6-methoxy-8-(4-(trifluoromethyl)cyclohex-1-en-1-yl)quinoline-3-carboxamide (Compound 115) (0.026 g, 0.066 mmol, Yield: 21.65%).

¹H NMR (DMSO-d₆, 400 MHz): δ ppm, 9.06 (d, J=2.0 Hz, 1H), 8.65 (d, J=2.4, Hz, 1H), 8.53 (d, J=7.6 Hz, 1H), 7.38 (d, J=2.8, Hz, 1H), 7.23 (d, J=2.8, Hz, 1H), 5.89 (br s, 1H), 4.19-4.10 (m, 1H), 3.95 (s, 3H), 2.77-2.67 (m, 3H), 2.49-2.44 (m, 1H), 2.33-2.23 (m, 1H), 2.08-2.05 (m, 1H), 1.72-1.62 (m, 1H), 1.16 (d, J=7.2 Hz, 6H).

HPLC (Method A): 9.46 min, 97.20%, 254.0 nm,

LCMS (Method A): 2.576 min, 97.02%, 254.0 nm, MS: ES+393.4 (M+1).

### Example 107—Synthesis of (R)-8-(4,4-difluorocyclohex-1-en-1-yl)-N-(1-hydroxypropan-2-yl)quinoline-3-carboxamide (Compound 116)

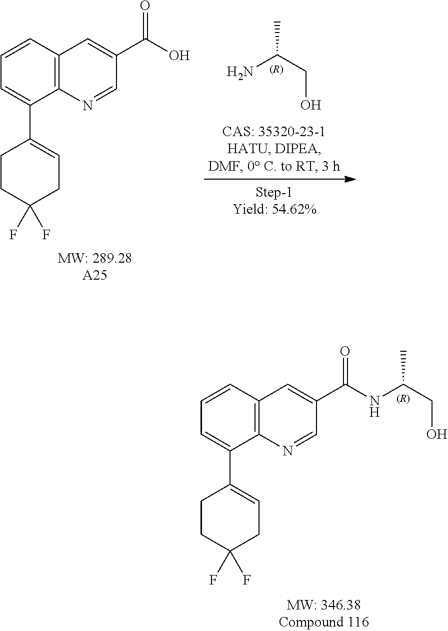

### Step-1

A stirred solution of 8-(4,4-difluorocyclohex-1-en-1-yl)quinoline-3-carboxylic acid (A25) (0.1 g, 0.34 mmol, 1.0 eq.) in DMF (1.0 mL) was prepared in 10 mL of glass vial at room temperature. To this reaction solution, DIPEA (0.18 mL, 1.03 mmol, 3.0 eq.) and HATU (0.19 g, 0.51 mmol, 1.5 eq.) were added at 0° C. under nitrogen atmosphere. After 30 min of stirring at 0° C., (R)-2-aminopropan-1-ol (CAS: 35320-23-1) (0.025 g, 0.34 mmol, 1.0 eq.) was added and the resulting reaction mixture was stirred from 0° C. to RT for 1 h. TLC indicated the completion of reaction; the resulting reaction mixture was diluted with cold water (5 mL) and extracted with EtOAc (3×5 mL). The combined organic layers were dried over Na₂SO₄, filtered and concentrated under reduced pressure to afford crude. The obtained crude material was purified by flash column chromatography using silica (230-400 mesh) as a stationary phase (desired product eluted in 70% EtOAc in Hexane) afforded (R)-8-(4,4-difluorocyclohex-1-en-1-yl)-N-(1-hydroxypropan-2-yl)quinoline-3-carboxamide (Compound 116) (0.065 g, 0.18 mmol, Yield: 54.62%).

¹H NMR (DMSO-d₆, 400 MHz): δ ppm, 9.26 (d, J=2.0 Hz, 1H), 8.81 (d, J=2.0 Hz, 1H), 8.48 (d, J=8.0 Hz, 1H), 8.02 (dd, J=7.2 Hz, 2.0 Hz, 1H), 7.67-7.63 (m, 2H), 5.76 (br s, 1H), 4.80 (t, J=6.0 Hz, 1H), 4.08-4.06 (m, 1H), 3.52-3.49 (m, 1H), 3.41-3.37 (m, 1H), 2.90 (br s, 2H), 2.80-2.75 (m, 2H), 2.25-2.18 (m, 2H), 1.17 (d, J=6.8 Hz, 3H).

¹H NMR (MeOD, 400 MHz): δ ppm, 9.26 (d, J=2.4 Hz, 1H), 8.77 (d, J=2.4 Hz, 1H), 7.98 (dd, J=8.0 Hz, 1.6 Hz, 1H), 7.69-7.62 (m, 2H), 5.75 (br s, 1H), 4.28 (q, J=6.0 Hz, 1H), 3.67 (t, J=5.2 Hz, 2H), 2.89-2.86 (m, 2H), 2.78 (m, 2H), 2.34-2.24 (m, 2H), 1.30 (t, d=6.4 Hz, 3H). Note: —CONH and —OH proton exchanged in MeOD NMR.

LCMS (Method A): 1.948 min, 97.77%, 254 nm, MS: ES+347.1 (M+1) HPLC (Method A): 7.04 min, 96.92%, 254 nm

Chiral HPLC (Method A): 3.27 min, 100%, 242 nm

### Example 108—Synthesis of (R)-8-(4,4-difluorocyclohex-1-en-1-yl)-N-(1-(oxazol-2-yl)ethyl)quinoline-3-carboxamide (Compound 117)

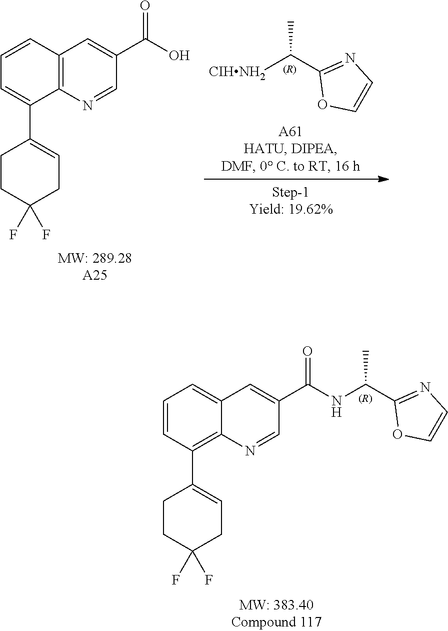

### Step-1

To a stirred solution of 8-(4,4-difluorocyclohex-1-en-1-yl)quinoline-3-carboxylic acid (A25) (0.1 g, 0.345 mmol, 1.0 eq.) in DMF (1.0 mL) was added HATU (0.262 g, 0.691 mmol, 2.0 eq.) and DIPEA (0.16 mL, 1.037 mmol, 3.0 eq.) at 0° C. After 10 min of stirring, (R)-1-(oxazol-2-yl)ethan-1-amine hydrochloride (A61) (0.046 g, 0.414 mmol, 1.2 eq.) was added and the resulting reaction mixture was stirred at RT for 16 h. TLC indicated the completion of reaction; the resulting reaction mixture was poured in cold water (5 mL) to obtain precipitate. The precipitate was filtered through Buchner funnel and washed with water (20 mL) and dried under reduced pressure and purified by column chromatography (desired product eluted in 0-2% MeOH in DCM) to afford (R)-8-(4,4-difluorocyclohex-1-en-1-yl)-N-(1-(oxazol-2-yl)ethyl)quinoline-3-carboxamide (Compound 117) (0.026 g, 0.067 mmol, Yield: 19.62%).

¹H NMR (MeOD, 400 MHz): δ ppm, 9.28 (s, 1H), 8.82 (s, 1H), 8.08 (s, 1H), 7.98 (d, J=8.0 Hz, 1H), 7.92 (s, 1H), 7.70-7.63 (m, 2H), 7.18 (s, 1H), 5.75 (br s, 1H), 5.50-5.48 (m, 1H), 2.88 (br s, 2H), 2.77 (t, J=14.8 Hz, 2H), 2.32-2.25 (m, 2H), 1.72 (d, J=6.8 Hz, 3H).

LCMS (Method A): 2.190 min, 100%, 254 nm, MS: ES+384.17 (M+1)

HPLC (Method A): 7.81 min, 100%, 254 nm

Chiral HPLC: 5.14 min, 100%, 240 nm

### Example 109—Synthesis of(S)-8-(4,4-difluorocyclohex-1-en-1-yl)-N-(1-(oxazol-2-yl)ethyl)quinoline-3-carboxamide (Compound 118)

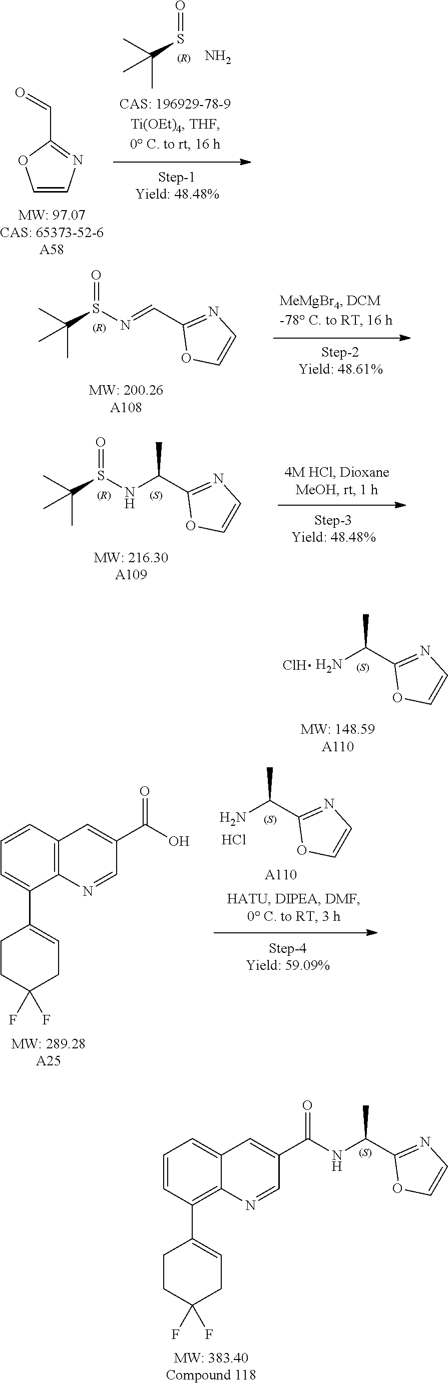

### Step-1

A stirred solution of oxazole-2-carbaldehyde (A58) (CAS: 65373-52-6) (0.4 g, 4.120 mmol, 1.0 eq) in THF (4 mL, 10 v) was prepared in 50 mL three neck round bottom flask at room temperature. To this solution was added (R)-2-methylpropane-2-sulfinamide (CAS: 196929-78-9) (0.599 g, 4.944 mmol, 1.2 eq) at same temperature. Then dropwise addition of Ti(OEt)₄ (1.88 g, 8.240 mmol, 2.0 eq) at 0° C. The resulting reaction mixture was stirred at RT for 16 h. The reaction was monitored by TLC (50% EtOAc in Hexane as a mobile phase), After completion, the resulting reaction mixture was dilute with brine solution (3 mL), EtOAc (20 mL) and stirred for 15 min. After 15 min, solid material was filtered through celite bed and washed with EtOAc (10 mL). The organic layer was separated, dried over anhy. Na₂SO₄, filtered and concentrated under reduced pressure to obtained the crude. The crude material was purified by flash column chromatography using silica 230-400 mesh size and desired product eluted in 22% EtOAc in Hexane to afford (R,E)-2-methyl-N-(oxazol-2-ylmethylene) propane-2-sulfinamide (A108) brown sticky liquid (0.4 g, 1.997 mmol, Yield: 48.48).

¹H NMR (DMSO-d₆, 400 MHz): δ ppm 8.45 (s, 1H), 8.28 (s, 1H), 7.63 (s, 1H), 1.19 (s, 9H).

LCMS (Method A): 1.298 min, 100%, 254.0 nm, MS: ES+201.0 (M+1)

### Step-2

A stirred solution of (R,E)-2-methyl-N-(oxazol-2-ylmethylene) propane-2-sulfinamide (A108) (0.4 g, 1.997 mmol, 1.0 eq) in DCM (8 mL, 20 v) was prepared in 50 mL three neck round bottom flask at −78° C. To this reaction mixture dropwise added MeMgBr (0.8 mL, 2.936 mmol, 1.2 eq) at −78° C. and stirred for 1.5 h. at same temperature. After 1.5 h, reaction mixture was warm to RT and again stirred for another 1.5 h. Then second time dropwise addition of MeMgBr (0.8 mL, 2.936 mmol, 1.2 eq) at −78° C. and stirred 1.5 h. at same temperature. After 1.5 h, again reaction mixture was warm to RT and stirred for another 1.5 h. Then third time dropwise addition of MeMgBr (0.4 mL, 1.468 mmol, 1.2 eq) at −78° C. and resulting reaction mixture was stirred at RT for another 16 h. The TLC indicated (100% EtOAc as a mobile phase) completion of reaction after 16 h of stirring at RT. The resulting reaction mixture was quenched with aq. NH₄Cl solution (10 mL) and extracted with EtOAc (2×20 mL). The combine organic layer was separated, dried over anhy. Na₂SO₄, filtered and concentrated under reduced pressure to obtained crude material. The crude material was purified by trituration with 20% Diethyl ether and n-pentane to afford (R)-2-methyl-N—((S)-1-(oxazol-2-yl)ethyl) propane-2-sulfinamide (A109) Light yellow solid (0.21 g, 0.970 mmol, Yield: 48.61).

¹H NMR (DMSO-d₆, 400 MHz): δ ppm 8.07 (s, 1H), 7.15 (s, 1H), 5.94 (d, J=7.6 Hz, 1H), 4.51-4-48 (m, 1H), 1.49 (d, J=6.8 Hz, 3H), 1.08 (s, 9H).

LCMS (Method A): 1.379 min, 100%, 210 nm, MS: ES+217.0 (M+1)

### Step-3

A stirred solution of (R)-2-methyl-N—((S)-1-(oxazol-2-yl)ethyl) propane-2-sulfinamide (A109) (0.1 g, 0.462 mmol, 1.0 eq) in MeOH (0.8 mL) was prepared in 10 mL glass vail at room temperature. To this reaction mixture was dropwise added 4M HCl in Dioxane (0.24 mL, 0.970 mmol, 2.1 eq) at RT. The resulting reaction mixture was stirred for 16 h. at same temperature. The reaction was monitored by TLC (100% EtOAc as a mobile phase) which confirmed that the reaction got completed after 16 h of stirring at room temperature. The resulting reaction mixture was directly concentrated under reduced pressure to obtained 0.075 g material as a(S)-1-(oxazol-2-yl)ethan-1-amine hydrochloride yellowish sticky liquid (A110) (0.075 g, 0.668 mmol, Yield: 48.48%).

¹H NMR (DMSO-d₆, 400 MHz): δ ppm 8.85 (br s, 3H), 8.24 (s, 1H), 7.32 (s, 1H), 4.66-4.64 (m, 1H), 1.56 (d, J=6.8 Hz, 3H).

LCMS (Method A): 0.185 min, 100.0%, 210 nm, MS: ES+113.1 (M+1)

### Step-4

A stirred solution of 8-(4,4-difluorocyclohex-1-en-1-yl)quinoline-3-carboxylic acid (A25) (0.05 g, 0.172 mmol, 1.0 eq.) in DMF (0.8 mL) was prepared in 10 mL of glass vial at room temperature. To this reaction solution, DIPEA (0.15 mL, 0.864 mmol, 5.0 eq.) and HATU (0.098 g, 0.259 mmol, 1.5 eq.) were added under nitrogen atmosphere. After 10 min of stirring, (S)-1-(oxazol-2-yl)ethan-1-amine hydrochloride (A110) (0.026 g, 0.172 mmol, 1.0 eq.) was added and the resulting reaction mixture was stirred at RT for 3 h. TLC indicated the completion of reaction; the resulting reaction mixture was poured onto cold water (20 mL) and extracted with ethyl acetate (20×2), organic layer was dried over Na₂SO₄ and concentrated under reduce pressure to obtain crude product which was purified by normal phase column chromatography using silica (100-200 mesh) desired product was eluted at 42% ethyl acetate in hexane to afford(S)-8-(4,4-difluorocyclohex-1-en-1-yl)-N-(1-(oxazol-2-yl)ethyl)quinoline-3-carboxamide (Compound 118) (0.039 g, 0.101 mmol, Yield: 59.09%).

¹H NMR (DMSO-d₆, 400 MHz): δ ppm, 9.36 (d, J=7.6 Hz, 1H), 9.28 (d, J=2.0 Hz, 1H), 8.87 (d, J=2.0 Hz, 1H), 8.08 (s, 1H), 8.03 (dd, J=7.6, 2.0 Hz, 1H), 7.69-7.63 (m, 2H), 7.19 (s, 1H), 5.77 (br s, 1H), 5.40-5.36 (m, 1H), 2.90 (br s, 2H), 2.83-2.27 (m, 2H), 2.27-2.17 (m, 2H), 1.61 (d, J=7.2 Hz, 3H).

LCMS (Method A): 2.181 min, 98.92%, 245 nm, MS: ES+384.12 (M+1)

HPLC (Method A): 7.78 min, 99.09%, 210 nm

### Example 110—Synthesis of N-((1H-pyrazol-5-yl)methyl)-8-(4,4-difluorocyclohex-1-en-1-yl)quinoline-3-carboxamide (Compound 119)

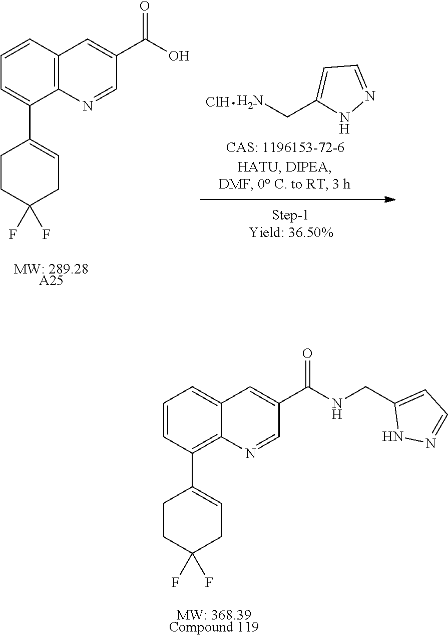

### Step-1

A stirred solution of 8-(4,4-difluorocyclohex-1-en-1-yl)quinoline-3-carboxylic acid (A25) (0.05 g, 0.17 mmol, 1.0 eq.) in DMF (1.0 mL) was prepared in 10 mL of glass vial at room temperature. To this reaction solution, DIPEA (0.15 mL, 0.86 mmol, 5.0 eq.) and HATU (0.098 g, 0.25 mmol, 1.5 eq.) were added at 0° C. under nitrogen atmosphere. After 30 min of stirring at 0° C., (1H-pyrazol-5-yl) methenamine hydrochloride (CAS: 11961153-72-6) (0.023 g, 0.17 mmol, 1.0 eq.) was added at same temperature. Then the resulting reaction mixture was stirred from 0° C. to RT for 3 h. TLC indicated the completion of reaction; the resulting reaction mixture was diluted with cold water (5 mL) and extracted with EtOAc (3×5 mL). The combined organic layers were dried over Na₂SO₄, filtered and concentrated under reduced pressure to afford crude. The obtained crude material was purified by flash column chromatography using silica (230-400 mesh) as a stationary phase (2% MeOH in DCM) to afford as a N-((1H-pyrazol-5-yl)methyl)-8-(4,4-difluorocyclohex-1-en-1-yl)quinoline-3-carboxamide (Compound 119) (0.023 g, 0.062 mmol, Yield: 36.50%).

¹H NMR (DMSO-d₆, 400 MHz): δ ppm, 12.64 (s, 1H), 9.28 (d, J=2.0 Hz, 2H), 8.85 (d, J=2.0 Hz, 1H), 8.01 (d, J=7.6 Hz, 1H), 7.67-7.41 (m, 3H), 6.23 (s, 1H), 5.76 (br s, 1H), 4.54 (s, 2H), 2.89 (s, 2H), 2.83-2.73 (m, 2H), 2.27-2.21 (m, 2H),

LCMS (Method A): 2.020 min, 100%, 254 nm, MS: ES+369.2 (M+1)

HPLC (Method A): 7.02 min, 97.98%, 254 nm

### Example 111—Synthesis of 8-(4,4-difluorocyclohex-1-en-1-yl)-N-((4-methyl-1H-pyrazol-5-yl)methyl)quinoline-3-carboxamide (Compound 120)

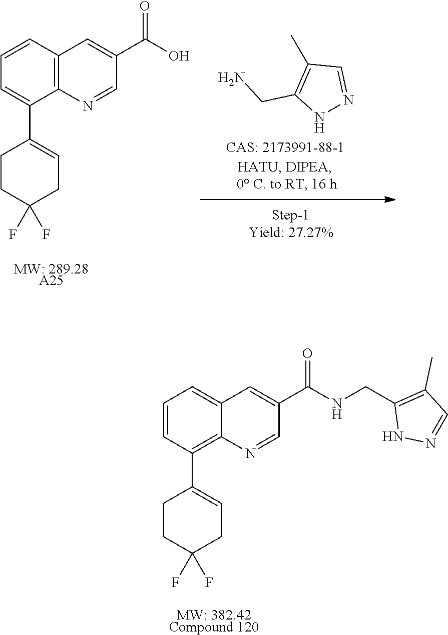

### Step-1

A stirred solution of 8-(4,4-difluorocyclohex-1-en-1-yl)quinoline-3-carboxylic acid (A25) (0.05 g, 0.17 mmol, 1.0 eq.) in DMF (1.0 mL) was prepared in 10 mL of glass vial at room temperature. To this reaction solution, DIPEA (0.09 mL, 0.51 mmol, 3.0 eq.) and HATU (0.098 g, 0.25 mmol, 1.5 eq.) were added at 0° C. under nitrogen atmosphere. After 30 min of stirring at 0° C., (4-methyl-1H-pyrazol-5-yl) methanamine (CAS: 2173991-88-1) (0.031 g, 0.17 mmol, 1.0 eq.) was added at same temperature. Then the resulting reaction mixture was stirred from 0° C. to RT for 16 h. TLC indicated the completion of reaction; the resulting reaction mixture was diluted with cold water (5 mL) and extracted with EtOAc (3×10 mL). The combined organic layers were dried over Na₂SO₄, filtered and concentrated under reduced pressure to afford the crude product. The obtained crude material was purified by RFC using (0-30% water in ACN) to afford as an 8-(4,4-difluorocyclohex-1-en-1-yl)-N-((4-methyl-1H-pyrazol-5-yl)methyl)quinoline-3-carboxamide (Compound 120) (0.018 g, 0.047 mmol, Yield: 27.27%).

¹H NMR (DMSO-d₆, 400 MHz): δ ppm, 12.39 (s, 1H), 9.28 (s, 1H), 9.10 (s, 1H) 8.84 (s, 1H), 8.00 (d, J=6.8 Hz, 1H), 7.67-7.61 (m, 2H), 7.45 (br s, 1H), 5.75 (br s, 1H), 4.52 (d, J=4.8 Hz, 2H), 2.89 (br s, 2H), 2.82-2.75 (m, 2H), 2.25-2.18 (m, 2H), 2.02 (s, 3H).

¹H NMR (MeOD, 400 MHz): δ ppm, 9.27 (d, J=2.0 Hz, 1H), 8.77 (d, J=2.0 Hz, 1H), 7.97 (d, J=6.8 Hz, 1H), 7.69-7.62 (m, 2H), 7.42 (br s, 1H), 5.75 (br s, 1H), 4.68 (s, 2H), 2.87 (br s, 2H), 2.81-2.74 (m, 2H), 2.32-2.25 (m, 2H), 2.15 (s, 3H). Note: —CONH and —NH protons exchanged in MeOD NMR.

LCMS (Method A): 2.101 min, 95.53%, 254 nm, MS: ES+383.2 (M+1) HPLC (Method A): 7.36 min, 98.50%, 254 nm

### Example 112—Synthesis of(S)-8-(4,4-difluorocyclohex-1-en-1-yl)-N-(1-methoxy propan-2-yl)quinoline-3-carboxamide (Compound 121)

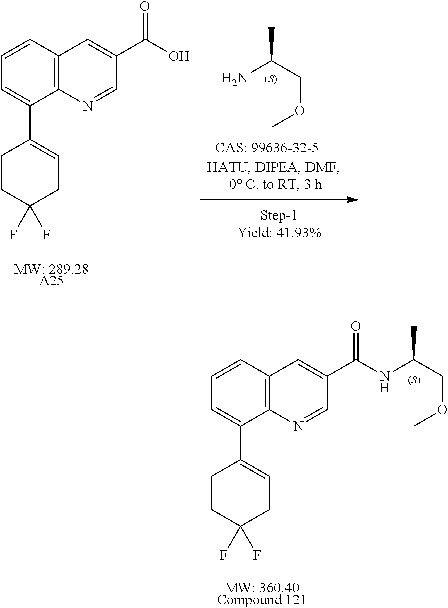

### Step-1

A stirred solution of 8-(4,4-difluorocyclohex-1-en-1-yl)quinoline-3-carboxylic acid (A25) (0.1 g, 0.34 mmol, 1.0 eq.) in DMF (1.0 mL) was prepared in 10 mL of glass vial at room temperature. To this reaction solution, DIPEA (0.18 mL, 1.03 mmol, 3.0 eq.) and HATU (0.19 g, 0.51 mmol, 1.5 eq.) were added at 0° C. under nitrogen atmosphere. After 30 min of stirring at 0° C., (S)-1-methoxypropan-2-amine (CAS: 99636-32-5) (0.025 g, 0.34 mmol, 1.0 eq.) was added at same temperature. The resulting reaction mixture was stirred from 0° C. to RT for 3 h. TLC indicated the completion of reaction; the resulting reaction mixture was diluted with cold water (5 mL) and extracted with EtOAc (3×5 mL). The combined organic layers were dried over Na₂SO₄, filtered and concentrated under reduced pressure to afford crude. The obtained crude material was purified by flash column chromatography using silica (230-400 mesh) as a stationary phase (desired product eluted in 30% EtOAc in Hexane) to afford(S)-8-(4,4-difluorocyclohex-1-en-1-yl)-N-(1-methoxypropan-2-yl)quinoline-3-carboxamide (Compound 121) (0.052 g, 0.14 mmol, Yield: 41.93%).

¹H NMR (DMSO-d₆, 400 MHz): δ ppm, 9.25 (d, J=2.0 Hz, 1H), 8.80 (d, J=2.0 Hz, 1H), 8.61 (d, J=8.0 Hz, 1H), 8.03 (dd, J=7.6, 2.0 Hz, 1H), 7.67-7.62 (m, 2H), 5.76 (s, 1H), 4.27-4.24 (m, 1H), 3.47-3.40 (m, 2H), 3.26 (s, 3H), 2.90 (br s, 2H), 2.83-2.75 (m, 2H), 2.25-2.18 (m, 2H), 1.19 (d, J=6.8 Hz, 3H).

¹H NMR (MeOD, 400 MHz): δ ppm, 9.24 (d, J=2.4 Hz, 1H), 8.75 (d, J=2.0 Hz, 1H), 7.98 (dd, J=8.0 Hz, 1.6 Hz, 1H), 7.69-7.62 (m, 2H), 5.75 (br s, 1H), 4.42-4.40 (m, 1H), 3.57-3.53 (m, 1H), 3.42 (s, 3H), 3.50-3.46 (m, 1H), 2.89-2.86 (m, 2H), 2.81-2.74 (m, 2H), 2.32-2.25 (m, 2H), 1.31 (d, J=6.8 Hz, 3H). Note: —CONH proton exchanged in MeOD NMR.

LCMS (Method A): 2.178 min, 95.62%, 254 nm, MS: ES+361.2 (M+1)

HPLC (Method A): 8.17 min, 100%, 254 nm

Chiral HPLC: 2.96 min, 100% 242 nm

### Example 113—Synthesis of 8-(4,4-difluorocyclohex-1-en-1-yl)-N-((5-oxopyrrolidin-2-yl)methyl)quinoline-3-carboxamide (Compound 122)

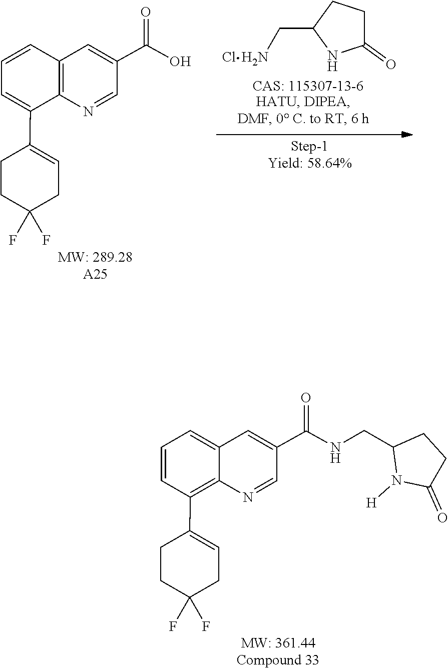

### Step-1

To a stirred solution of 8-(4,4-difluorocyclohex-1-en-1-yl)quinoline-3-carboxylic acid (A25) (0.05 g, 0.172 mmol, 1.0 eq.) in DMF (1.0 mL) was added HATU (0.098 g, 0.259 mmol, 1.5 eq.) and DIPEA (0.066 mL, 0.518 mmol, 3.0 eq.) at 0° C. After 10 min of stirring, 5-(aminomethyl) pyrrolidin-2-one hydrochloride (CAS: 115307-13-6) (0.028 g, 0.190 mmol, 1.1 eq.) was added and the resulting reaction mixture was stirred at RT for 6 h. TLC indicated the completion of reaction; the resulting reaction mixture was poured in cold water (5 mL) to obtain the precipitate. The precipitate was filtered through Buchner funnel and washed with water (20 mL) and dried under reduced pressure then crude obtained was purified by column chromatography (and eluted with 0-2% MeOH in DCM to afford 8-(4,4-difluorocyclohex-1-en-1-yl)-N-((5-oxopyrrolidin-2-yl)methyl)quinoline-3-carboxamide (Compound 122) (0.039 g, 0.101 mmol, Yield: 58.64%).

¹H NMR (DMSO-d₆, 400 MHz): δ ppm, 9.27 (d, J=2.0 Hz, 1H), 8.87-8.82 (m, 2H), 8.03 (dd, J=2.0, 7.2 Hz, 1H), 7.83 (s, 1H), 7.68-7.62 (m, 2H), 5.76 (br s, 1H), 3.76 (t, J=6.0 Hz, 1H), 3.40-3.32 (m, 2H), 2.90-2.83 (m, 2H), 2.79-2.67 (m, 2H), 2.26-2.10 (m, 5H), 1.82-1.79 (m, 1H).

LCMS (Method A): 1.914 min, 98.09%, 254 nm, MS: ES+386.17 (M+1)

HPLC (Method A): 6.52 min, 98.44%, 254 nm

Chiral HPLC: Peak-1 3.67 min, 49.95% 240 nm; Peak-2 4.80 min, 50.04%, 240 nm

### Example 114—Synthesis of 8-(4,4-difluorocyclohex-1-en-1-yl)-N-((6-oxo-1,6-dihydropyridin-2-yl)methyl)quinoline-3-carboxamide (Compound 123)

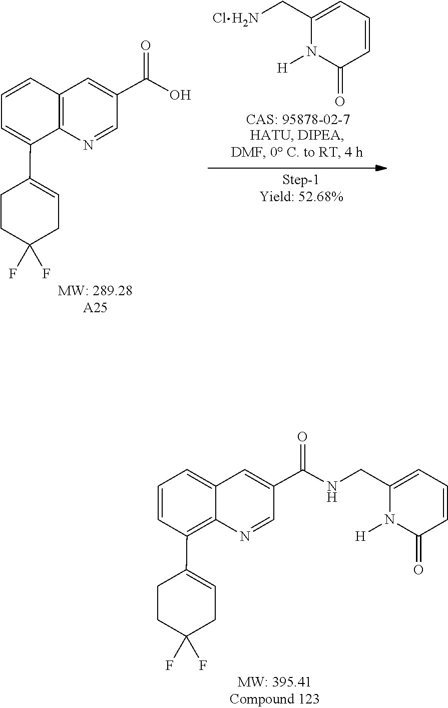

### Step-1

To a stirred solution of 8-(4,4-difluorocyclohex-1-en-1-yl)quinoline-3-carboxylic acid (A25) (0.05 g, 0.172 mmol, 1.0 eq.) in DMF (1.0 mL) was added HATU (0.098 g, 0.259 mmol, 1.5 eq.) and DIPEA (0.066 g, 0.518 mmol, 3.0 eq.) at 0° C. After 10 min of stirring, 6-(aminomethyl)pyridin-2 (1H)-one hydrochloride (CAS: 95878-02-7) (0.030 g, 0.190 mmol, 1.1 eq.) was added and the resulting reaction mixture was stirred at RT for 4 h. TLC indicated the completion of reaction; the resulting reaction mixture was poured into cold water (5 mL) to obtain precipitate. The precipitate was filtered through Buchner funnel and washed with water (20 mL) then concentrated under reduced pressure. The obtained crude was purified by column chromatography on silica gel (230-400 mesh) (desired product eluted in 0-4% MeOH in DCM) to afford 8-(4,4-difluorocyclohex-1-en-1-yl)-N-((6-oxo-1,6-dihydropyridin-2-yl)methyl)quinoline-3-carboxamide (Compound 123) (0.036 g, 0.091 mmol, Yield: 52.68%).

¹H NMR (DMSO-d₆, 400 MHz): δ ppm, 11.63 (br s, 1H), 9.34-9.30 (m, 2H), 8.88 (d, J=2.0 Hz, 1H), 8.05 (dd, J=7.6, 1.6 Hz, 1H), 7.69-7.64 (m, 2H), 7.40-7.38 (m, 1H), 6.22-6.13 (m, 2H), 5.77 (br s, 1H), 4.39 (d, J=5.6 Hz, 2H), 2.95-2.87 (m, 2H), 2.83-2.76 (m, 2H), 2.28-2.17 (m, 2H).

LCMS (Method A): 1.954 min, 98.46%, 254 nm, MS: ES+396.27 (M+1)

HPLC (Method A): 6.53 min, 98.41%, 254 nm

### Example 115—Synthesis of 8-(4,4-difluorocyclohex-1-en-1-yl)-N-((2-oxo-1,2-dihydro pyridin-3-yl)methyl)quinoline-3-carboxamide (Compound 124)

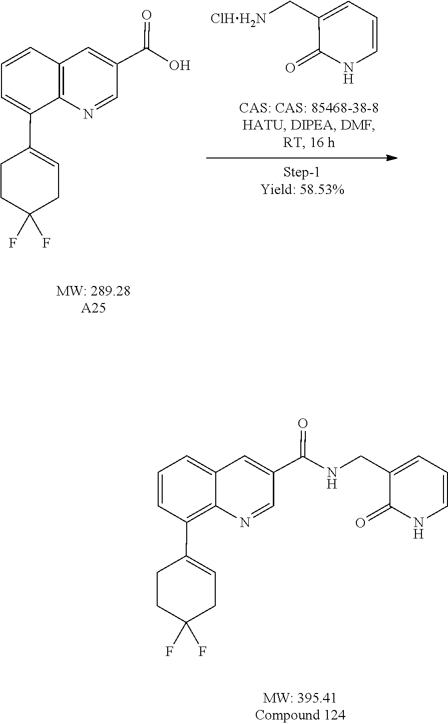

### Step-1

A stirred solution of 8-(4,4-difluorocyclohex-1-en-1-yl)quinoline-3-carboxylic acid (A25) (0.05 g, 0.17 mmol, 1.0 eq.) in DMF (1 mL) was prepared in 10 mL of glass vial at room temperature. To this reaction solution, DIPEA (0.089 mL, 0.51 mmol, 3.0 eq.) and HATU (0.098 g, 0.25 mmol, 1.5 eq.) were added at same temperature under nitrogen atmosphere. After 30 min of stirring at 0° C., CAS: 85468-38-8 (0.033 g, 0.20 mmol, 1.2 eq.) was added and the resulting reaction mixture was stirred from 0° C. to RT for 16 h. TLC indicated the completion of reaction; the resulting reaction mixture was poured into cold water (5 mL) to obtain the precipitate. The obtained precipitate was filtered through Buchner funnel, washed with water (5 mL×2) and concentrated under reduced pressure to afford 8-(4,4-difluorocyclohex-1-en-1-yl)-N-((2-oxo-1,2-dihydropyridin-3-yl)methyl)quinoline-3-carboxamide (Compound 124) (0.040 g, 0.10 mmol, Yield: 58.53%).

¹H NMR (DMSO-d₆, 400 MHz): δ ppm 11.7 (br s, 1H), 9.31 (d, J=2.4 Hz, 1H), 9.15 (t, J=6.8 Hz, 1H), 8.88 (d, J=2.0 Hz, 1H), 8.03 (dd, J=1.6, 6.0 Hz, 1H) 7.67-7.64 (m, 2H), 7.38 (d, J=6.4 Hz, 1H), 7.32 (d, J=5.2 Hz, 1H), 6.43 (t, J=6.4 Hz, 1H), 5.77 (br s, 1H), 4.31 (d, J=5.2 Hz, 2H), 2.90 (br s, 2H), 2.81-2.79 (m, 2H), 2.24-2.21 (m, 2H).

¹H NMR (MeOD, 400 MHz): δ ppm 9.29 (d, J=2.0 Hz, 1H), 8.81 (d, J=2.4 Hz, 1H), 7.99 (dd, J=1.6, 6.4 Hz, 1H), 7.70-7.63 (m, 3H), 7.40 (dd, J=4.8 Hz, 1.6, 1H), 6.43 (t, J=6.4 Hz, 1H), 5.75 (br s, 1H), 4.25 (s, 2H), 2.90-2.87 (m, 2H), 2.81-2.74 (m, 2H), 2.34-2.24 (m, 2H). Note: —CONH proton exchanged in MeOD NMR.

LCMS (Method A): 1.95 min, 100%, 210.0 nm, MS: ES+396.2 (M+1)

HPLC (Method A): 6.63 min, 97.89%, 210.0 nm

### Example 116—Synthesis of 8-(4,4-difluorocyclohex-1-en-1-yl)-N-(1-(hydroxy methyl)cyclopropyl)quinoline-3-carboxamide (Compound 125)

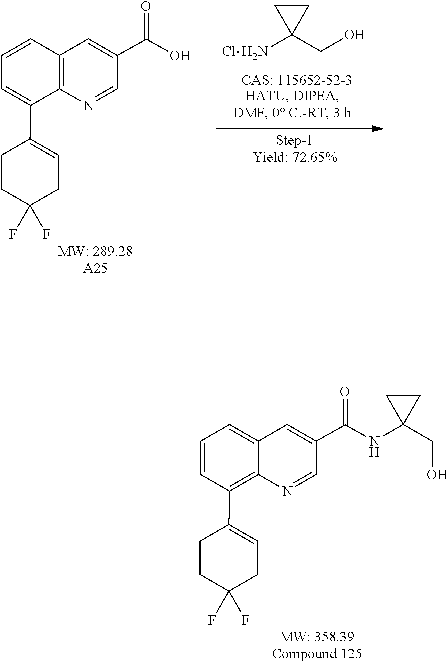

### Step-1

A stirred solution of 8-(4,4-difluorocyclohex-1-en-1-yl)quinoline-3-carboxylic acid (A25) (0.05 g, 0.17 mmol, 1.0 eq.) in DMF (0.5 mL) was prepared in 10 mL of glass vial and cooled to 0° C. To this reaction solution, HATU (0.09 g, 0.26 mmol, 1.5 eq.) and DIPEA (0.07 g, 0.51 mmol, 3.0 eq.) was added followed by addition of 1-amino-cyclopropanemethanol hydrochloride (CAS: 115652-52-3) (0.02 g, 0.17 mmol, 1.0 eq.) and the resulting reaction mixture was stirred at RT for 3 h. TLC indicated the completion of reaction; the resulting reaction mixture was poured onto cold water (20 mL) and precipitated out which were filtered and washed with water (50 mL) and dried under reduced pressure to obtain the crude which was purified by reverse phase column chromatography on silica gel (230-400 mesh), desired product eluted in 45% water in acetonitrile to afford 8-(4,4-difluorocyclohex-1-en-1-yl)-N-(1-(hydroxymethyl)cyclopropyl)quinoline-3-carboxamide (Compound 125) (0.045 g, 0.12 mmol, Yield: 72.65%).

¹H NMR (DMSO-d₆, 400 MHz): δ ppm, 9.25 (d, J=2.0 Hz, 1H), 9.03 (s, 1H), 8.82 (d, J=2.0 Hz, 1H), 7.99 (dd, J=7.2, 1.6 Hz, 1H), 7.67-7.61 (m, 2H), 5.77 (s, 1H), 4.84 (t, J=4.8 Hz, 1H), 3.58 (d, J=4.8 Hz, 2H), 2.89 (m, 2H), 2.79-2.70 (m, 2H), 2.26-2.17 (m, 2H), 0.81-0.76 (m, 4H)

LCMS (Method A): 1.982 min, 98.29%, 254.0 nm, MS: ES+359.12 (M+1)

HPLC (Method B): 7.00 min, 98.60%, 254.0 nm

### Example 117—Synthesis of (R)-8-(4,4-difluorocyclohex-1-en-1-yl)-N-(4-(methylamino)-4-oxobutan-2-yl)quinoline-3-carboxamide (Compound 126)

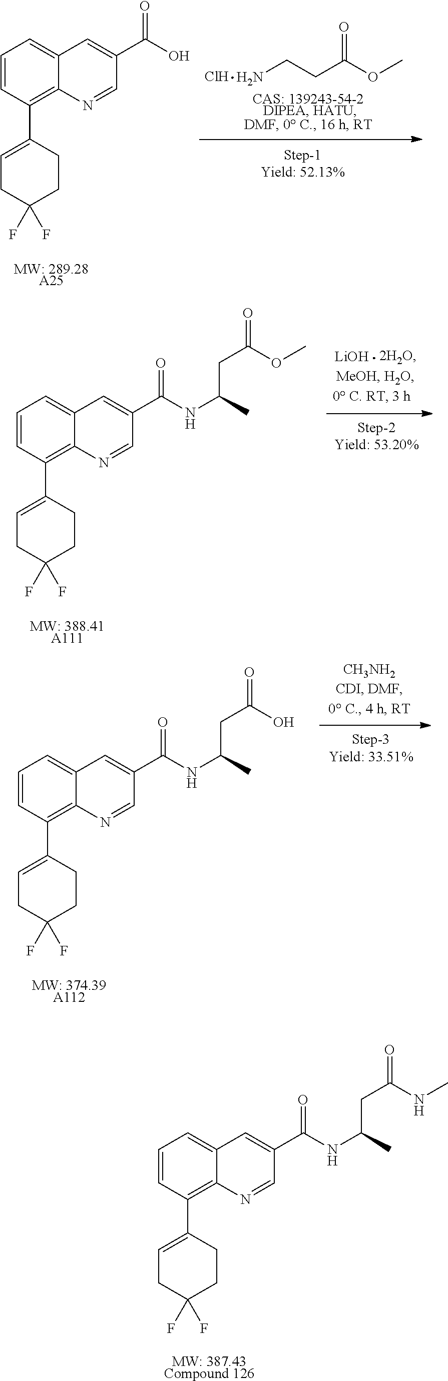

### Step-1

A stirred solution of 8-(4,4-difluorocyclohex-1-en-1-yl)quinoline-3-carboxylic acid (A25) (0.5 g, 1.73 mmol, 1.0 eq.) in DMF (5 mL) was prepared in 50 mL of single neck at room temperature. To this reaction solution, DIPEA (0.8 mL, 5.19 mmol, 3.0 eq.) and HATU (0.98 g, 2.59 mmol, 1.5 eq.) were added at same temperature under nitrogen atmosphere. After 30 min of stirring at 0° C., CAS: 139243-54-2 (R)-Methyl 3-aminobutanoate hydrochloride (0.29 g, 1.90 mmol, 1.1 eq.) was added at same temperature. The resulting reaction mixture was stirred from 0° C. to RT for 16 h. TLC indicated the completion of reaction; the resulting reaction mixture was poured in cold water (5 mL) to obtain precipitate. Precipitate was filtered through Buchner funnel, washed with water and dried over high vacuum to afford as methyl (R)-3-(8-(4,4-difluorocyclohex-1-en-1-yl)quinoline-3-carboxamido) butanoate (A111) (0.35 g, 0.90 mmol, Yield: 52.13%).

¹H NMR (DMSO-d₆, 400 MHz): δ ppm 9.23 (d, J=2.0 Hz, 1H), 8.77 (d, J=2.0 Hz, 1H), 8.70 (d, J=7.6 Hz, 1H), 8.03 (d, J=5.6 Hz, 1H), 7.67-7.62 (m, 2H), 5.76 (br s, 1H), 4.59-4.39 (m, 1H), 3.64 (s, 3H), 2.90 (br s, 2H), 2.82-2.72 (m, 2H), 2.70-2.66 (m, 2H), 2.62-2.54 (m, 2H), 2.25-2.18 (m, 2H), 1.24 (d, J=6.8 Hz, 3H).

LCMS (Method A): 2.21 min, 93%, 254.0 nm, MS: ES+389.3 (M+1)

### Step-2

A stirred solution of methyl (R)-3-(8-(4,4-difluorocyclohex-1-en-1-yl)quinoline-3-carboxamido) butanoate (A111) (0.35 g, 0.90 mmol, 1.0 eq.) in THF (3.5 Ml) was prepared in 25 Ml of single neck at room temperature. To this reaction solution, aq. solution of LiOH in water (3.5 Ml) (0.113 g, 2.70 mmol, 3.0 eq.) was added and resulting reaction mixture was stirred at RT for 3 h. TLC indicated the completion of reaction; the resulting reaction mixture was neutralized with solution of citric acid to obtain precipitate. Precipitate was filtered through Buchner funnel, washed with water and dried over high vacuum to afford as a (R)-3-(8-(4,4-difluorocyclohex-1-en-1-yl)quinoline-3-carboxamido)butanoic acid (A112) (0.25 g, 0.66 mmol, Yield: 53.20%).

¹H NMR (MeOD, 400 MHz): δ ppm 9.24 (d, J=2.0 Hz, 1H), 8.73 (d, J=1.6 Hz, 1H), 7.97 (d, J=8.0 Hz, 1H), 7.68-7.62 (m, 2H), 5.75 (br s, 1H), 4.57 (q, J=6.8 Hz, 1H), 2.87-2.55 (m, 7H), 2.32-2.25 (m, 2H), 1.38 (d, J=6.8 Hz, 3H). Note: —COOH proton might be exchanged in MeOD NMR.

LCMS (Method A): 1.99 min, 93%, 254.0 nm, MS: ES+375.1 (M+1)

### Step-3

A stirred solution of (R)-3-(8-(4,4-difluorocyclohex-1-en-1-yl)quinoline-3-carboxamido) butanoic acid (A112) (0.100 g, 0.26 mmol, 1.0 eq.) in DMF (1 mL) was prepared in 25 mL of single neck at room temperature. To this reaction solution, CDI (0.086 g, 0.53 mmol, 2.0 eq.) were added at RT. The methylamine solution (2M in THF) (10 vol) was added dropwise into the reaction. The resulting reaction mixture stirred at RT for 4 h. TLC indicated the completion of reaction; the resulting reaction mixture was quenched with ice-cold water (10 mL) and extracted with EtOAc (3×10 mL). The combined organic layers were dried over Na₂SO₄ and concentrated under reduced pressure to afford crude. The obtained crude material was purified by flash column chromatography using silica (230-400 Mesh) as stationary phase (90% Ethyl acetate in hexane) to afford (R)-8-(4,4-difluorocyclohex-1-en-1-yl)-N-(4-(methylamino)-4-oxobutan-2-yl)quinoline-3-carboxamide (Compound 126) (0.045 g, 0.11 mmol, Yield: 33.51%).

¹H NMR (MeOD, 400 MHz): δ ppm 9.23 (d, J=2.4 Hz, 1H), 8.73 (d, J=2.4 Hz, 1H), 7.98 (dd, J=1.6, 7.6 Hz, 1H), 7.69-7.62 (m, 2H), 5.75 (br s, 1H), 4.58-4.53 (m, 1H), 2.89-2.86 (m, 2H), 2.81-2.78 (m, 2H), 2.75 (s, 3H), 2.62-2.57 (m, 1H), 2.51-2.46 (m, 1H), 2.32-2.25 (m, 2H), 1.36 (d, J=6.8 Hz, 3H). Note: —CONH proton exchanged in MeOD NMR.

LCMS (Method A): 1.948 min, 100%, 210.0 nm, MS: ES+388.4 (M+1)

HPLC (Method A): 6.83 min, 100%, 254 nm

### Example 118—Synthesis of N-(cyanomethyl)-8-(4,4-difluorocyclohex-1-en-1-yl)quinoline-3-carboxamide (Compound 127)

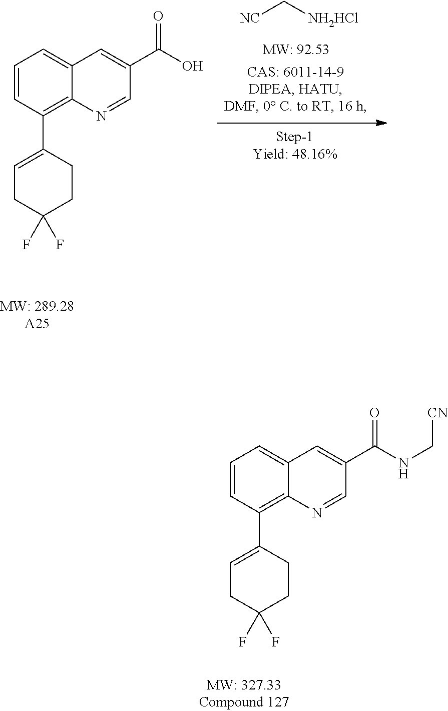

### Step-1

To a stirred solution of A25 (0.20 g, 0.691 mmol, 1.0 eq.) in DMF (2.0 mL) was added HATU (0.394 g, 1.037 mmol, 1.5 eq.) and DIPEA (0.57 mL, 2.074 mmol, 3.0 eq.) at 0° C. After 10 min of stirring, 2-aminoacetonitrile hydrochloride (CAS: 6011-14-9) (0.06397 g, 0.6914 mmol, 1.0 eq.) was added and reaction stirred for 16 h. TLC indicated the completion of reaction; the resulting reaction mixture was diluted with NaHCO₃ (50 mL) and extracted with EtOAc (3×20 mL). The combined organic layers extracted with EtOAc (2×50 mL), dried over Na₂SO₄ and concentrated under reduced pressure to afford crude. The crude was purified by manual column chromatography using silica (230-400 mesh) as stationary phase (gradient elution 35% Ethyl acetate in hexane) to afford N-(cyanomethyl)-8-(4,4-difluorocyclohex-1-en-1-yl)quinoline-3-carboxamide (Compound 127) (0.109 g, 0.333 mmol, Yield 48.16%)

¹H NMR (DMSO, 400 MHz): δ ppm 9.57 (t, J=5.2 Hz, 1H), 9.27 (d, J=2.4 Hz, 1H), 8.87 (d, J=2.4 Hz, 1H), 8.05 (dd, J=1.6, 7.6 Hz, 1H), 7.71-7.65 (m, 2H), 5.76 (d, J=3.6 Hz, 1H), 4.42 (d, J=5.2 Hz, 2H), 2.90 (t, J=5.2 Hz, 2H), 2.80 (t, J=14.0 Hz, 2H), 2.26-2.19 (m, 2H).

LCMS (Method A): 2.122 min, 254 nm, 100%, m/z=328.2 (M+H)+

HPLC (Method A): 7.76 min, 254 nm, 100%

### Example 119—Synthesis of N-(1-(1H-pyrazol-5-yl)ethyl)-8-(4,4-difluorocyclohex-1-en-1-yl)quinoline-3-carboxamide (Compound 128)

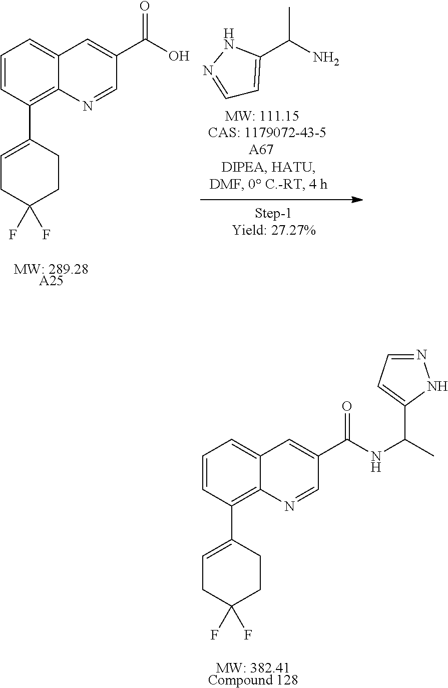

### Step-1

To a stirred solution of 8-(4,4-difluorocyclohex-1-en-1-yl)quinoline-3-carboxylic acid (A25) (0.15 g, 0.519 mmol, 1.0 eq.) in DMF (1.0 mL) was added HATU (0.295 g, 0.778 mmol, 1.5 eq.) and DIPEA (0.27 mL, 0.1.556 mmol, 3.0 eq.) followed by addition of (1H-pyrazol-5-yl)ethan-1-amine (A67) (CAS: 1179072-43-5) (0.063 g, 0.57 mmol, 1.1 eq.) and the resulting reaction mixture was stirred at RT for 4 h. TLC indicated the completion of reaction; the resulting reaction mixture was poured in cold water (5 mL) to obtain precipitate. The precipitate was filtered through Buchner funnel and washed with water (20 mL) and dried over high vacuum and purified by column chromatography and eluted with 50% EtOAC in Hexane to afford N-(1-(1H-pyrazol-5-yl)ethyl)-8-(4,4-dimethylcyclohex-1-en-1-yl)quinoline-3-carboxamide (Compound 128) (0.029 g, 0.076 mmol, Yield: 14.62%).

¹H NMR (DMSO-d₆, 400 MHz): δ ppm, 12.58 (br s, 1H) 9.28 (d, J=2.4, 1H), 9.02 (s, 1H), 8.82 (d, J=2.0 Hz, 1H), 7.97 (t, J=5.2 Hz, 1H), 7.63-7.60 (m, 3H), 6.25 (s, 1H), 5.76 (br s, 1H), 5.34 (br s, 1H), 2.90-2.89 (m, 2H), 2.83-2.69 (m, 2H), 2.32-2.19 (m, 2H), 1.52 (d, J=7.2 Hz, 3H).

LCMS (Method A): 2.053 min, 96.82%, 254 nm, MS: ES+383.3 (M+1)

HPLC (Method A): 7.32 min, 95.76%, 254 nm

### Example 120—Synthesis of (R)—N-(4-amino-4-oxobutan-2-yl)-8-(4,4-difluoro cyclohex-1-en-1-yl)quinoline-3-carboxamide (Compound 129)

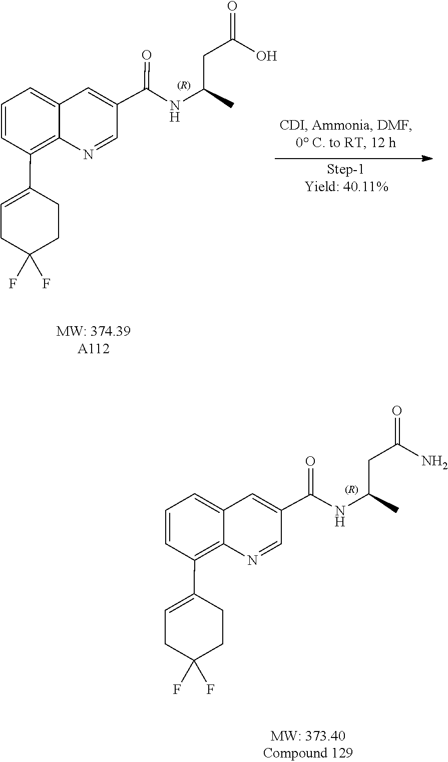

### Step-1

A stirred solution of (R)-3-(8-(4,4-difluorocyclohex-1-en-1-yl)quinoline-3-carboxamido) butanoic acid (A112) (0.10 g, 0.26 mmol, 1.0 eq.) in DMF (1.0 mL) was prepared in 25 mL of single neck RBF at room temperature. To this reaction solution was added CDI (0.086 g, 0.53 mmol, 2.0 eq.) at RT. Then dropwise addition of aq. ammonia solution (10 vol) and reaction mixture was stirred at RT for 16 h. TLC indicated the completion of reaction; the resulting reaction mixture was quenched with ice-cold water (10 mL) and extracted with EtOAc (3×10 mL). The combined organic layers were dried over Na₂SO₄, filtered and concentrated under reduced pressure to afford crude product. The obtained crude material was purified by flash column chromatography (desired product eluted in 90% Ethyl acetate in hexane) to afford (R)—N-(4-amino-4-oxobutan-2-yl)-8-(4,4-difluorocyclohex-1-en-1-yl)quinoline-3-carboxamide (Compound 129) (0.040 g, 0.10 mmol, Yield: 40.11%).

¹H NMR (MeOD, 400 MHz): δ ppm 9.24 (d, J=2.4 Hz, 1H), 8.74 (d, J=2.4 Hz, 1H), 7.98 (dd, J=2.0 Hz & 8.0 Hz, 1H), 7.70-7.62 (m, 2H), 5.75 (br s, 1H), 4.59-4.57 (m, 1H), 2.89-2.86 (m, 2H), 2.81-2.74 (m, 2H), 2.66-2.61 (m, 1H), 2.53-2.48 (m, 1H), 2.32-2.25 (m, 2H), 1.38 (d, J=6.8 Hz, 3H). Note: —CONH and —CONH₂ protons exchanged in MeOD NMR.

LCMS (Method A): 1.895 min, 100%, 210.0 nm, MS: ES+374.4 (M+1)

HPLC (Method A): 6.59 min, 100%, 210.0 nm

### Example 121—Synthesis of (R)-8-(4,4-difluorocyclohex-1-en-1-yl)-N-(1-hydroxy butan-2-yl)quinoline-3-carboxamide (Compound 130)

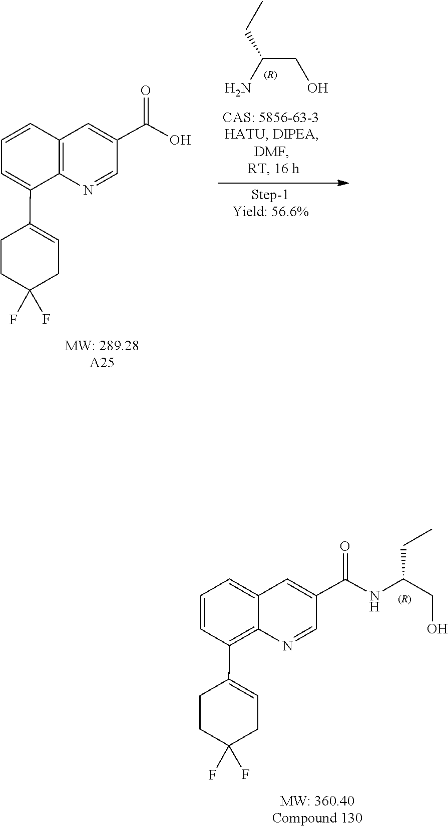

### Step-1

To a stirred solution of A25 (0.1 g, 0.34 mmol, 1.0 eq.) in DMF (2.0 mL) was added DIPEA (0.133 g, 1.03 mmol, 3.0 eq.) and HATU (0.197 g, 0.51 mmol, 1.5 eq.) at RT under N₂ atmosphere and stirred reaction for 10 min. After 10 min, addition of CAS: 5856-63-6 (R)-(−)-2-Amino-1-butanol (0.033 g, 0.38 mmol, 1.1 eq.) and resulting reaction was stirred at RT for 16 h. TLC indicated the completion of reaction; the resulting reaction mixture was diluted with water (10 mL) and extracted with EtOAc (3×20 mL). The combined organic layer was dried over anhy. Na₂SO₄, filtered and concentrated under reduced pressure to afford crude product. The crude product was purified by manual column chromatography (desired product eluted in 100% Ethyl acetate) to afford (R)-8-(4,4-difluorocyclohex-1-en-1-yl)-N-(1-hydroxybutan-2-yl)quinoline-3-carboxamide (Compound 130) (0.068 g, 0.18 mmol, Yield: 56.6%).

¹H NMR (DMSO-d₆, 400 MHz): δ ppm 9.27 (s, 1H), 8.82 (s, 1H), 8.38 (d, J=8.4 Hz, 1H), 8.02 (d, J=7.2 Hz, 1H), 7.63 (t, J=7.6 Hz, 2H), 5.76 (br s, 1H), 4.74 (t, J=5.6 Hz, 1H), 3.93 (d, J=5.2 Hz, 1H), 3.53-3.46 (m, 2H), 2.90 (br s, 2H), 2.79-2.76 (m, 2H), 2.24-2.18 (m, 2H), 1.70-1.68 (m, 1H), 1.50-1.47 (m, 1H), 0.91 (t, J=7.2 Hz, 3H).

¹H NMR (MeOD, 400 MHz): δ ppm 9.27 (d, J=2.0 Hz, 1H), 8.78 (d, J=2.4 Hz, 1H), 7.98 (dd, J=7.6 Hz & 7.6 Hz, 1H), 7.69-7.64 (m, 2H), 5.75 (s, 1H), 4.13-4.10 (m, 1H), 3.70-3.68 (m, 2H), 2.87 (m, 2H), 2.83-2.74 (m, 2H), 2.34-2.24 (m, 2H), 1.83-1.77 (m, 1H), 1.67-1.59 (m, 1H), 1.07 (t, J=7.6 Hz, 3H).

Note: —CONH and —OH protons exchanged in MeOD NMR.

LCMS (Method-A): 2.047 min, 95.99%, 210 nm, MS: ES+361.2 (M+1)

HPLC (Method-A): 7.33, 95.88%, 220 nm

Chiral HPLC (Method): 3.60, 100%, 240 nm

### Example 122—Synthesis of (R)-8-(4,4-difluorocyclohex-1-en-1-yl)-N-(2-hydroxy propyl)quinoline-3-carboxamide (Compound 131)

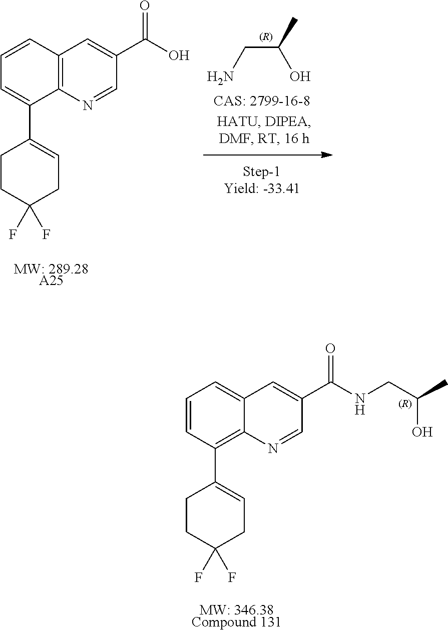

### Step-1

To a stirred solution of A25 (0.1 g, 0.345 mmol, 1.0 eq.) in DMF (1 mL) were added HATU (0.262 g, 0.691 mmol, 2.0 eq.) and DIPEA (0.133 g, 1.03 mmol, 3.0 eq.) at RT under nitrogen atmosphere. Followed by addition of (R)-1-aminopropan-2-ol (CAS: No: 2799-16-8) (0.028 g, 0.380 mmol, 1.1 eq.) at 0° C. and then stirred at RT for 16 h. TLC indicated the completion of reaction; the resulting reaction mixture was diluted with water (10 ml) extracted with EA (20 mL×2), combined organic layers were concentrated under reduced pressure to obtain the crude which was purified by column chromatography using silica gel (230-400 mesh) (50% ethyl acetate in hexane as gradient) to afford (R)-8-(4,4-difluorocyclohex-1-en-1-yl)-N-(2-hydroxypropyl)quinoline-3-carboxamide (Compound 131) (0.040 g, 0.115 mmol, Yield: 33.41%).

¹H NMR (DMSO-d₆, 400 MHz): δ ppm, 9.28 (d, J=2.0 Hz, 1H), 8.85-8.81 (m, 2H), 8.0 (q, J=2.0 Hz, 7.2 Hz 1H), 7.67-7.62 (m, 2H), 5.76 (br s, 1H), 4.85 (br s, 1H), 3.84 (d, J=8.0 Hz, 1H), 3.29-3.21 (m, 2H), 2.90 (br s, 2H), 2.79 (t, J=14.0 Hz, 2H), 2.25-2.17 (m, 2H), 1.11 (d, J=6.4 Hz, 3H).

¹H NMR (MeOD, 400 MHz): δ ppm, 9.27 (d, J=2.0 Hz, 1H), 8.78 (d, J=2.4 Hz, 1H), 7.99 (d, J=8 Hz, 1H), 7.69-7.63 (m, 2H), 5.75 (s, 1H), 4.06-4.02 (m, 1H), 3.54-3.49 (m, 1H), 3.43-3.38 (m, 2H), 2.28 (br s, 2H), 2.78 (t, J=14.4 Hz, 2H), 2.34-2.24 (m, 2H), 1.26 (d, J=6.4 Hz, 3H). Note: —CONH and —OH proton exchanged in MeOD NMR.

LCMS (Method A): 1.96, 100%, 254 nm, MS: ES+347.3 (M), (M+1)

HPLC (Method-A): 7.30 min, 100%, 210 nm

### Example 123—Synthesis of(S)-8-(4,4-difluorocyclohex-1-en-1-yl)-N-(2-hydroxypropyl)quinoline-3-carboxamide (Compound 132)

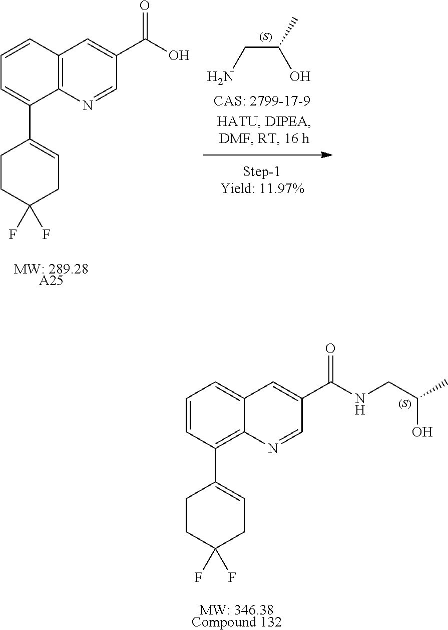

### Step-1

To the stirred solution of A25 (0.1 g, 0.345 mmol, 1.0 eq.) in DMF (1 mL) was added HATU (0.196 g, 0.517 mmol, 1.5 eq.) and DIPEA (0.15 mL, 1.035 mmol, 3.0 eq.) at RT under Nitrogen atmosphere, followed by addition of(S)-1-aminopropan-2-ol CAS: 2799-17-9 (0.028 g, 0.380 mmol, 1.1 eq.) and then stirred at RT for 16 h. The resulting reaction mixture was stirred at RT for 16 h. Water (30 mL) was added and extracted with EtOAc (30 mL) three times, organic layer was dried over Na₂SO₄ and evaporate under vacuum to get crude product. The crude was purified by column chromatography using silica gel (230-400 mesh) (eluting with 45-50% Ethyl Acetate in Hexane) to afford(S)-8-(4,4-difluorocyclohex-1-en-1-yl)-N-(2-hydroxypropyl)quinoline-3-carboxamide (Compound 132) (0.016 g, 0.046 mmol, Yield: 11.97%)

¹H NMR (DMSO-d₆, 400 MHz): δ ppm 9.27 (d, J=2 Hz, 1H), 8.82 (d, J=2 Hz, 1H), 8.77 (t, J=5.6 Hz, 1H), 8.02 (dd, J=2.0 Hz, 7.6 Hz, 1H), 7.67-7.62 (m, 2H), 5.76 (br s, 1H), 4.82 (d, J=4.8 Hz, 1H), 3.85-3.82 (m, 1H), 3.29-3.25 (m, 2H), 2.90 (br s, 2H), 2.79 (t, J=14.8 Hz, 2H), 2.27-2.17 (m, 2H), 1.10 (d, J=2.8 Hz, 3H).

¹H NMR (MeOD, 400 MHz): δ ppm 9.27 (d, J=2 Hz, 1H), 8.79 (d, J=2.4 Hz, 1H), 7.99 (dd, J=1.6 Hz, 7.6 Hz, 1H), 7.70-7.63 (m, 2H), 5.75 (s, 1H), 4.04-4.02 (m, 1H), 3.54-3.49 (m, 1H), 3.42 (d, J=7.2 Hz, 1H), 2.88 (t, J=5.2 Hz, 2H), 2.83-2.74 (m, 2H), 2.32-2.25 (m, 2H), 1.26 (d, J=6.4 Hz, 3H). Note: —CONH and —OH proton exchanged in MeOD NMR.

LCMS (Method A): 1.939 min, 95.12%, 254.0 nm MS: ES+347.21 [M+H]

HPLC (Method-A)-6.95 min, 96.10%, 210 nm

### Example 124—Synthesis of (R)—N-(2-acetamidopropyl)-8-(4,4-difluorocyclohex-1-en-1-yl)quinoline-3-carboxamide (Compound 133)

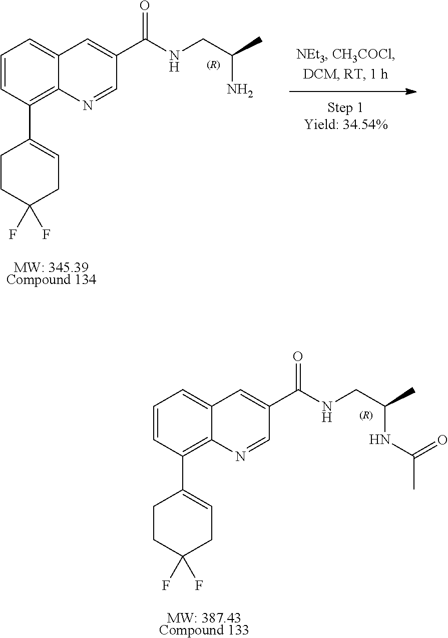

### Step-1

To a stirred solution of (R)—N-(2-aminopropyl)-8-(4,4-difluorocyclohex-1-en-1-yl)quinoline-3-carboxamide (Compound 134) (0.1 g, 0.28 mmol, 1.0 eq.) in DCM (5 mL) was added acetyl chloride (0.02 mL, 0.28 mmol, 1.0 eq.) and NEt₃ (0.12 mL, 0.86 mmol, 3.0 eq.) at RT under Nitrogen atmosphere and stirred for 1 h. TLC indicated the completion of reaction; the resulting reaction mixture was diluted with water (10 ml) and extracted with ethyl acetate (2×30 mL). The combined organic layers dried over Na₂SO₄ and concentrated under reduced pressure to afford crude. The crude was purified by manual column chromatography using silica (60-120 Mesh) as stationary phase (3% MDC in MeOH) to afford (R)—N-(2-acetamidopropyl)-8-(4,4-difluorocyclohex-1-en-1-yl)quinoline-3-carboxamide (Compound 133) (0.03 g, 0.077 mmol, 34.54%).

¹H NMR (MeOD, 400 MHz): δ ppm 9.24 (d, J=2.4 Hz, 1H), 8.72 (d, J=2.0 Hz, 1H), 7.99 (dd, J=8.0 Hz, 8.0 Hz, 1H), 7.70-7.63 (m, 2H), 5.75 (br s, 1H), 4.26-4.21 (m, 1H), 3.6-3.54 (m, 1H), 3.48-3.43 (m, 1H), 2.88 (t, J=6 Hz, 2H), 2.78-2.75 (m, 2H), 2.34-2.23 (m, 2H), 1.93 (s, 3H), 1.24 (d, J=6.8 Hz, 3H). Note: —CONH proton Exchanged in MeOD NMR.

¹H NMR (DMSO, 400 MHz): δ ppm 9.24 (d, J=2.0 Hz, 1H), 8.79 (m, 2H), 8.02 (dd, J=7.6, 1.6 Hz, 1H), 7.81 (d, J=8.0 Hz, 1H), 7.67-7.62 (m, 2H), 5.76 (s, 1H), 4.06-3.99 (m, 1H), 2.90 (br s, 2H), 2.79 (t, J=14.4 Hz, 2H), 2.26-2.17 (m, 2H), 1.80 (s, 3H), 1.08 (d, J=6.8 Hz, 3H).

LCMS (Method A) 1.94 min, 97.18%, 254.0 nm, MS: ES+388.37 (M), (M+1)

HPLC (Method A) 6.87 min, 96.26%, 220 nm

Chiral HPLC-3.73 min, 95.54%, 240.0 nm

### Example 125—Synthesis of (R)—N-(2-aminopropyl)-8-(4,4-difluorocyclohex-1-en-1-yl)quinoline-3-carboxamide (Compound 134)

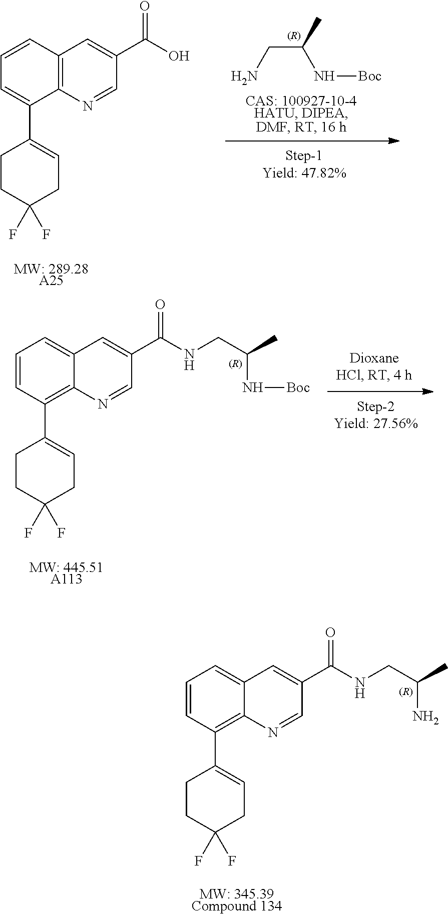

### Step-1

To a stirred solution of A25 (0.3 g, 1.03 mmol, 1.0 eq.) in DMF (2.0 mL) was added HATU (0.59 g, 1.55 mmol, 1.5 eq.) and DIPEA (0.53 mL, 3.11 mmol, 3.0 eq.) at RT under nitrogen atmosphere, followed by addition of(S)-1-aminopropan-2-ol CAS: 100927-10-4 (0.21 g, 1.24 mmol, 1.2 eq.) at 0° C. stirred at RT for 16 h. TLC indicated the completion of reaction; the resulting reaction mixture was diluted with cold water (30 mL) and extracted with EtOAc (30 mL) three times, organic layer was dried over Na₂SO₃ and evaporated under vacuum to get crude product. The crude was purified by column chromatography using silica gel (230-400 mesh) (eluting with 30% Ethyl Acetate in Hexane) to afford tert-butyl (R)-(1-(8-(4,4-difluorocyclohex-1-en-1-yl)quinoline-3-carboxamido) propan-2-yl) carbamate (A113) (0.22 g, 0.49 mmol, Yield 47.82%)

LCMS-(Method A): 2.47 min, 96.11%, 254.0 nm MS: ES+446.38 (M+1)

¹H NMR (DMSO, 400 MHz): δ ppm 9.25 (s, 1H), 8.78 (s, 2H), 8.00 (d, J=6.0 Hz, 1H), 7.64 (t, J=14.4 Hz, 2H), 6.81 (d, J=8.0 Hz, 1H), 5.76 (br s, 1H), 3.77 (br s, 1H), 3.15 (s, 2H), 2.89 (br s, 2H), 2.83-2.67 (m, 2H), 2.25-2.20 (m, 2H), 1.35 (s, 9H), 1.05 (d, J=6.4 Hz, 3H).

### Step-2

To a stirred solution of A113 (0.22 g, 0.49 mmol, 1.0 eq.) in dioxane (2.0 mL) was added 4M HCl in Dioxane (4 mL, 2.0 eq.) to the reaction mixture under the N₂ atmosphere. The resulting mixture was stirred at room temperature for 4 h. TLC indicated the completion of reaction; the reaction mixture was concentrated under reduced pressure to yield (R)—N-(2-aminopropyl)-8-(4,4-difluorocyclohex-1-en-1-yl)quinoline-3-carboxamide (Compound 134) (0.047 g, 0.140 mmol, Yield 27.56%)

¹H NMR (MeOD, 400 MHz): δ ppm 9.47 (s, 2H), 8.30 (d, J=8.0 Hz, 1H), 8.03 (d, J=7.2 Hz, 1H), 7.96 (t, J=8.0 Hz, 1H), 5.93 (s, 1H), 3.75-3.61 (m, 3H), 2.91-2.33 (m, 4H), 2.42-2.33 (m, 2H), 1.42 (d, J=6.8 Hz, 3H).

Note: —CONH and —NH₂ proton exchanged in MeOD NMR.

¹H NMR (DMSO, 400 MHz): δ ppm 9.33 (d, J=2.0 Hz, 1H), 9.15 (t, J=5.2 Hz, 1H), 8.92 (d, J=1.6 Hz, 1H), 8.04 (d, J=7.2 Hz, 4H), 7.70-7.64 (m, 2H), 5.77 (s, 1H), 2.90-2.67 (m, 4H), 2.28-2.17 (m, 2H), 1.25 (d, J=6.4 Hz, 3H). Note: —CH₂ proton merged in DMSO solvent peak.

LCMS—(Method A): 1.66 min, 98.72%, 254.0 nm

HPLC (Method A): 8.13 min, 97.58%, 254.0 nm

Chiral HPLC—5.65 min, 95.87%, 254.0 nm

### Example 126—Synthesis of 8-(4,4-difluorocyclohex-1-en-1-yl)-N-(3-hydroxypropyl)quinoline-3-carboxamide (Compound 135)

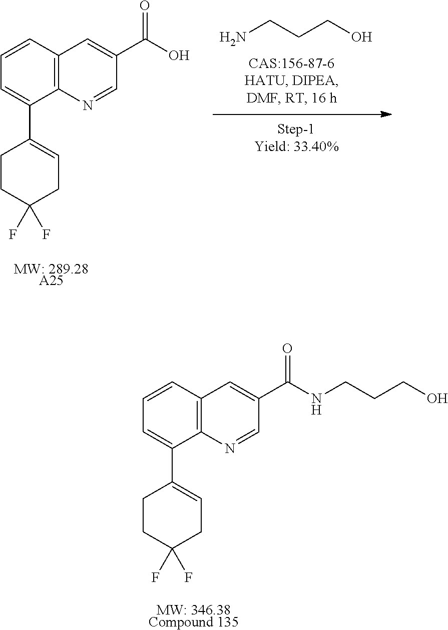

### Step-1

To a stirred solution of 8-(4,4-difluorocyclohex-1-en-1-yl)quinoline-3-carboxylic acid (A25) (0.150 g, 0.51 mmol, 1.0 eq.) in DMF (1.0 mL), was added DIPEA (0.20 g, 1.55 mmol, 3.0 eq.) and HATU (0.295 g, 0.77 mmol, 1.5 eq.) at 0° C. The reaction mixture was stirred under the N₂ atmosphere. After 15 min of stirring at 0° C., 3-aminopropan-1-ol (CAS: No: 156-87-6) (0.042 g, 0.57 mmol, 1.1 eq.) was added at same temperature and the resulting reaction mixture was stirred at room temperature for 16 h. TLC indicated the completion of reaction; the resulting reaction mixture was quenched with ice-cold water (15 mL) and extracted with ethyl acetate (15 mL×3). The combined organic layers were dried over Na₂SO₄, filtered and concentrated under reduced pressure to afford crude. The crude was purified by column chromatography using silica gel (230-400 Mesh) as a stationary phase (50% Ethyl acetate in Hexane) to afford 8-(4,4-difluorocyclohex-1-en-1-yl)-N-(3-hydroxypropyl)quinoline-3-carboxamide (Compound 135) (0.060 g, 0.173 mmol, Yield 33.40%).

¹H NMR (DMSO, 400 MHz): δ ppm 9.25 (s, 1H), 8.79 (s, 2H), 8.02 (d, J=6.0 Hz, 1H), 7.65-7.63 (m, 2H), 5.75 (br s, 1H), 4.52 (br s, 1H), 3.50 (d, J=4.8 Hz, 2H), 3.38 (d, J=5.6 Hz, 2H), 2.90 (s, 2H), 2.79-2.75 (m, 2H), 2.22 (s, 2H), 1.74-173 (m, 2H).

LCMS (Method A): 1.963 min, 95.50%, 254.0 nm, MS: ES+347.4 (M+1)

HPLC (Method B): 7.06 min, 100%, 254 nm.

### Example 127—Synthesis of 8-(4,4-difluorocyclohex-1-en-1-yl)-N-(3-hydroxy-2-methylpropyl)quinoline-3-carboxamide (Compound 136)

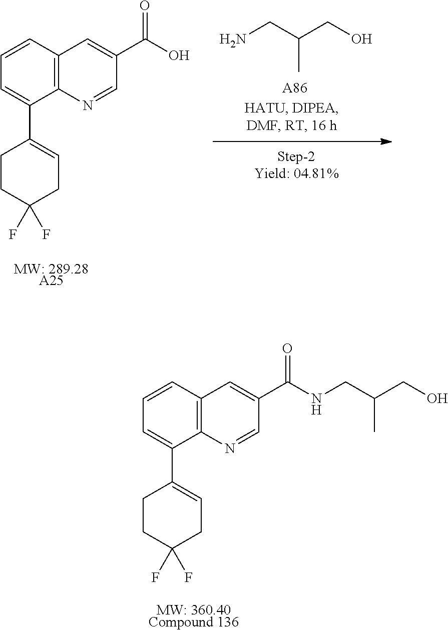

### Step-1

To a stirred solution of 8-(4,4-difluorocyclohex-1-en-1-yl)quinoline-3-carboxylic acid (A25) (0.1 g, 0.34 mmol, 1.0 eq.) in DMF (2.0 mL) was added HATU (0.19 g, 0.51 mmol, 1.5 eq.) and DIPEA (0.13 g, 1.037 mmol, 3.0 eq.) at 0° C. The reaction mixture was stirred under the N₂ atmosphere. After 15 min of stirring at 0° C., 3-amino-2-methylpropan-1-ol (A86) (0.034 g, 0.381 mmol, 1.1 eq.) was added and the resulting reaction mixture was stirred from 0° C. to room temperature for 16 h. TLC indicated the completion of reaction; the resulting reaction mixture was quenched with ice-cold water (3.0 mL) and extracted with ethyl acetate (10 mL×3). The combined organic layers were dried over Na₂SO₄ and concentrated under reduced pressure to afford crude. The crude material was purified by flash column chromatography using silica (230-400 Mesh) as stationary phase (50% Ethyl acetate in Hexane) to afford light brown gummy 8-(4,4-difluorocyclohex-1-en-1-yl)-N-(3-hydroxy-2-methylpropyl)quinoline-3-carboxamide (Compound 136) (0.012 g, 0.033 mmol, Yield: 4.81%).

¹H-NMR (DMSO, 400 MHz): δ ppm, 9.25 (d, J=2.4 Hz, 1H), 8.80 (d, J=2.0 Hz, 1H), 8.76 (t, J=5.6 Hz, 1H), 8.03 (dd, J=2.0, 7.2 Hz, 1H), 7.67-7.62 (m, 2H), 5.76 (s, 1H), 4.55 (t, J=5.2 Hz, 1H), 3.22-3.16 (m, 1H), 2.90 (br s, 2H), 2.83-2.75 (m, 2H), 2.27-2.17 (m, 2H), 1.90-1.85 (m, 1H), 0.90 (d, J=6.4 Hz, 3H). Note: 3H merged in moisture peak which is clearly visible in D₂O NMR.

LCMS (Method A): 2.047 min, 95.84%, 254.0 nm, MS: ES+361.3 (M+1)

HPLC (Method A): 7.31 min, 95.64%, 254 nm.

### Example 128—Synthesis of 8-(4,4-difluorocyclohex-1-en-1-yl)-N-(3-hydroxybutyl)quinoline-3-carboxamide (Compound 137)

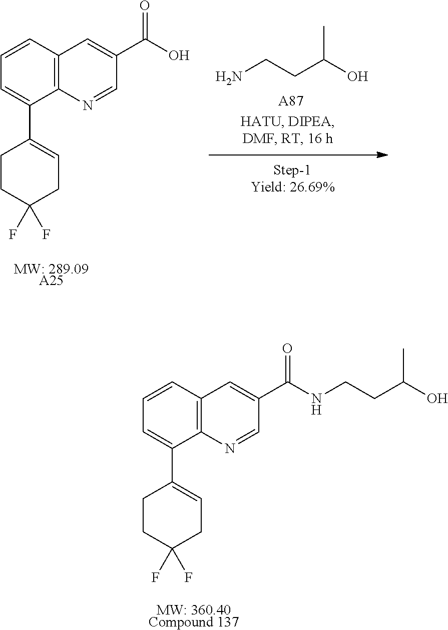

### Step-1

To a stirred solution of 8-(4,4-difluorocyclohex-1-en-1-yl)quinoline-3-carboxylic acid (A25) (0.1 g, 0.34 mmol, 1.0 eq.), in DMF (1.0 mL), was added DIPEA (0.130 g, 1.03 mmol, 3.0 eq.) and HATU (0.19 g, 0.51 mmol, 1.5 eq.), at 0° C. under the N₂ atmosphere. After 15 min of stirring at 0° C., 4-aminobutan-2-ol (A87) (0.046 g, 0.51 mmol, 1.5 eq.) was added at same temperature and the resulting reaction mixture was stirred at room temperature for 16 h. TLC indicated the completion of reaction; the resulting reaction mixture was quenched with ice-cold water (5 mL) and extracted with ethyl acetate (15 mL×3). The combined organic layers were dried over Na₂SO₄, filtered and concentrated under reduced pressure to afford crude. The crude was purified by column chromatography using silica (230-400 mesh) as stationary phase (40% Ethyl acetate in Hexane) to afford light brown 8-(4,4-difluorocyclohex-1-en-1-yl)-N-(3-hydroxy butyl)quinoline-3-carboxamide (Compound 137) (0.037 g, 0.10 mmol, Yield 26.69%).

¹H NMR (DMSO, 400 MHz): δ ppm, 9.25 (d, J=2.0 Hz, 1H), 8.79-8.75 (m, 2H), 8.02 (dd, J=2.0, 7.6 Hz, 1H), 7.67-7.62 (m, 2H), 5.76 (s, 1H), 4.55 (d, J=4.8 Hz, 1H), 3.73-3.70 (m, 1H), 3.42-3.37 (m, 2H), 2.90 (br s, 2H), 2.78 (t, J=14.2 Hz, 2H), 2.25-2.18 (m, 2H), 1.66-1.59 (m, 2H), 1.10 (d, J=6.4 Hz, 3H).

¹H NMR (MeOD, 400 MHz): δ ppm, 9.25 (d, J=2.0 Hz, 1H), 8.74 (d, J=2.0 Hz, 1H), 7.98 (dd, J=1.6, 8.0 Hz, 1H), 7.69-7.62 (m, 2H), 5.76 (br s, 1H), 3.94-3.86 (m, 1H), 3.64-3.50 (m, 2H), 2.89-2.86 (t, J=4.8 Hz, 2H), 2.78 (t, J=14.0 Hz, 2H), 2.34-2.19 (m, 2H), 1.88-1.72 (m, 2H), 1.25 (d, J=6.0 Hz, 3H). Note: —CONH and —OH proton exchanged in MeOD NMR.

LCMS (Method A): 2.033 min, 95.63%, 254.0 nm, MS: ES+361.4 (M+1) HPLC (Method B): 7.19 min, 95.11%, 220 nm Chiral HPLC: Peak-1: 5.68 min, 49.42%, 240 nm; Peak-2:6.38 min, 49.84%, 240 nm.

### Example 129—Synthesis of 8-(4,4-difluorocyclohex-1-en-1-yl)-N-isopropyl-6-methoxyquinoline-3-carboxamide (Compound 138)

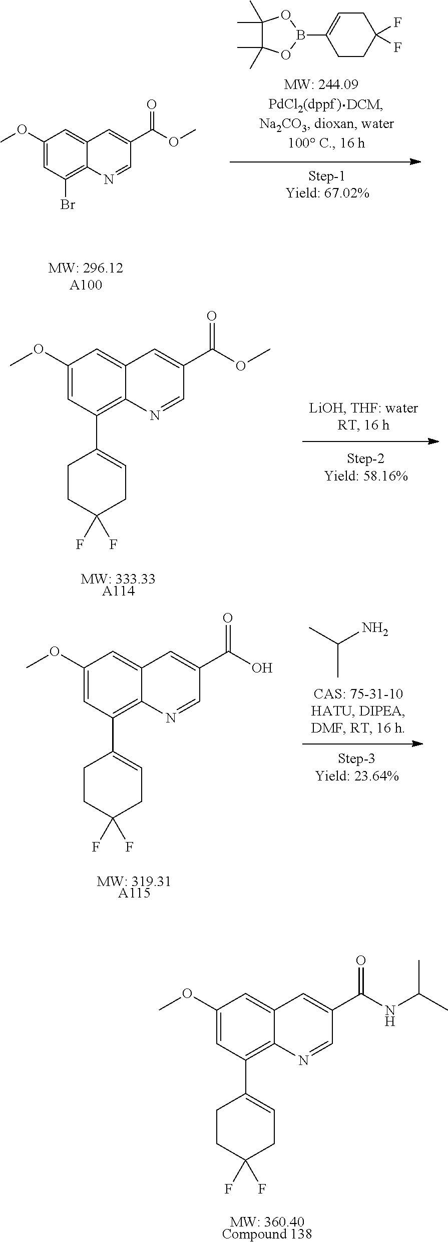

### Step-1

To a stirred solution of 2-(4,4-difluorocyclohex-1-en-1-yl)-4, 4, 5,5-tetramethyl-1, 3, 2-dioxaborolane (2.72 g, 11.14 mmol, 1.5 eq.), methyl 8-bromo-6-methoxyquinoline-3-carboxylate (A100) (2.20 g, 7.43 mmol, 1.0 eq.) and Na₂CO₃ (1.56 g, 14.86 mmol, 2.0 eq.) in Dioxane (40 mL) and water (10 mL) was added Pd(dppf)Cl₂·DCM (0.543 g, 0.743 mmol, 0.1 eq.) under N₂. The resulting mixture was stirred at 100° C. for 16 h. TLC indicated the completion of reaction; the resulting reaction mixture was diluted with water (200 mL) and extracted with EtOAc (2×250 mL). Organic phase was dried over Na₂SO₄ and concentrated under reduced pressure to afford the crude. The obtained crude material was purified by flash column chromatography using silica gel (230-400 mesh) as stationary phase (32% EtOAc in Hexane) afforded methyl 8-(4, 4-difluorocyclohex-1-en-1-yl)-6-methoxyquinoline-3-carboxylate (A114) (1.66 g, 4.98 mmol, Yield: 67.02%).

¹H NMR (DMSO-d₆, 400 MHz): δ ppm, 9.13 (d, J=2.4 Hz, 1H), 8.86 (d, J=2.8 Hz, 1H), 7.56 (d, J=2.8 Hz, 1H), 7.32 (d, J=2.8 Hz, 1H), 5.79 (br s, 1H), 3.94 (s, 3H), 3.92 (s, 3H), 2.88-2.85 (m, 2H), 2.81-2.74 (m, 2H), 2.74-2.59 (m, 2H).

LCMS (Method A): 2.654 min, 97.43%, 254.0 nm, MS: ES+334.3 (M+1)

### Step-2

A solution of methyl 8-(4,4-difluorocyclohex-1-en-1-yl)-6-methoxyquinoline-3-carboxylate (A114) (1.66 g, 4.98 mmol, 1.0 eq.) and LiOH (0.627 g, 14.94 mmol, 3.0 eq.) in THF:H₂O (10 mL, 7:3) was prepared at room temperature. The resulting mixture was stirred at RT for 16 h. TLC indicated the completion of reaction; the resulting reaction mixture was concentrated under reduced pressure and obtained residue was poured onto 1 N HCl solution, then filtered off to afford the desired product (A115) 8-(4, 4-difluorocyclohex-1-en-1-yl)-6-methoxyquinoline-3-carboxylic acid (0.925 g, 2.89 mmol, Yield: 58.16%).

¹H NMR (DMSO-d₆, 400 MHz): δ ppm, 13.46 (s, 1H), 9.13 (d, J=2.0 Hz, 1H), 8.83 (d, J=2.0 Hz, 1H), 7.54 (d, J=2.8 Hz, 1H), 7.30 (d, J=2.8 Hz, 1H), 5.78 (br s, 1H), 3.91 (s, 3H), 2.88-2.86 (m, 2H), 2.81-2.67 (m, 2H), 2.25-2.20 (m, 2H).

LCMS (Method A): 2.066 min, 100%, 254.0 nm, MS: ES+320.3 (M+1)

### Step-3

To a stirred solution of 8-(4, 4-difluorocyclohex-1-en-1-yl)-6-methoxyquinoline-3-carboxylic acid (A115) (0.100 g, 0.313 mmol, 1.0 eq.) in DMF (10 mL), was added isopropyl amine CAS: 75-31-0 (0.023 mg, 0.313 mmol, 1.0 eq.), DIPEA (0.122 g, 0.939 mmol, 3.0 eq.) and HATU (0.178 g, 0.470 mmol, 1.5 eq.). The resulting mixture was stirred at room temperature for 16 h. TLC indicated the completion of reaction; the resulting reaction mixture was diluted with ice cold water (50 mL), and solid obtained was filtered and vacuum dried to afford 8-(4, 4-difluorocyclohex-1-en-1-yl)-N-isopropyl-6-methoxyquinoline-3-carboxamide (Compound 138) (0.027 g, 0.074 mmol, 23.64%)

¹H NMR (DMSO-d₆, 400 MHz): δ ppm, 9.08 (d, J=2.0 Hz, 1H), 8.67 (d, J=2.0 Hz, 1H), 8.54 (d, J=7.4 Hz, 1H), 7.40 (d, J=2.8 Hz, 1H), 7.26 (d, J=2.8 Hz, 1H), 5.78 (br s, 1H), 4.17-4.12 (m, 1H), 3.91 (s, 3H), 2.89-2.88 (m, 2H), 2.78-2.73 (m, 2H), 2.24-2.17 (m, 2H), 1.21 (d, J=6.4 Hz, 6H).

HPLC (Method A): 8.29 min, 99.22%, 254.0 nm.

LCMS (Method A): 2.346 min, 97.75%, 254.0 nm, MS: ES+361.47 (M+1)

### Example 130—Synthesis of (R)-8-(4,4-difluorocyclohex-1-en-1-yl)-N-(1-hydroxypropan-2-yl)-6-methoxyquinoline-3-carboxamide (Compound 139)

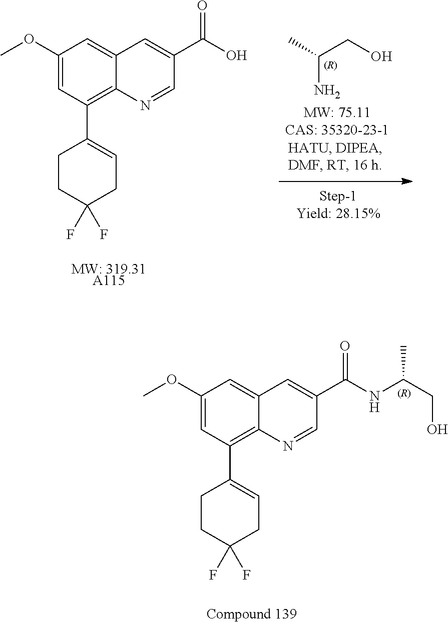

### Step-1

To a stirred solution of 8-(4,4-difluorocyclohex-1-en-1-yl)-6-methoxyquinoline-3-carboxylic acid (A115) (0.10 g, 0.313 mmol, 1.0 eq.) in DMF (15 mL), was added D-alinol CAS: 35320-23-1 (0.023 g, 0.313 mmol, 1.0 eq.), DIPEA (0.122 g, 0.939 mmol, 3.0 eq.) and HATU (0.178 g, 0.470 mmol, 1.5 eq.). The resulting mixture was stirred at room temperature for 16 h. TLC indicated the completion of reaction; the resulting reaction mixture was diluted with water (40 mL) and stirred for 15 mins. The solid obtained was filtered and concentrated under vacuum to afford (R)-8-(4,4-difluorocyclohex-1-en-1-yl)-N-(1-hydroxypropan-2-yl)-6-methoxyquinoline-3-carboxamide (Compound 139) (0.036 g, 0.088 mmol, 28.15%)

¹H NMR (DMSO-d₆ 400 MHz): δ ppm, 9.09 (d, J=2.0 Hz, 1H), 8.68 (d, J=2.0 Hz, 1H), 8.43 (d, J=8.0 Hz, 1H), 7.40 (d, J=2.8 Hz, 1H), 7.26 (d, J=2.8 Hz, 1H), 5.78 (br s, 1H), 4.78 (t, J=5.6 Hz, 1H), 4.10-4.05 (m, 1H), 3.91 (s, 3H), 3.53-3.47 (m, 1H), 3.47-3.33 (m, 1H), 2.88 (br s, 2H), 2.81-2.78 (m, 2H), 2.25-2.19 (m, 2H), 1.22 (d, J=6.8 Hz, 3H). Note: one proton merged in DMSO moisture peak.

HPLC (Method A): 7.104 min, 95.77%, 254.0 nm.

LCMS (Method A): 1.914 min, 97.10%, 254.0 nm, MS: ES+377.4 (M+1)

### Example 131—Synthesis of N-((1H-pyrazol-3-yl)methyl)-8-(4, 4-difluorocyclohex-1-en-1-yl)-6-methoxyquinoline-3-carboxamide (Compound 140)

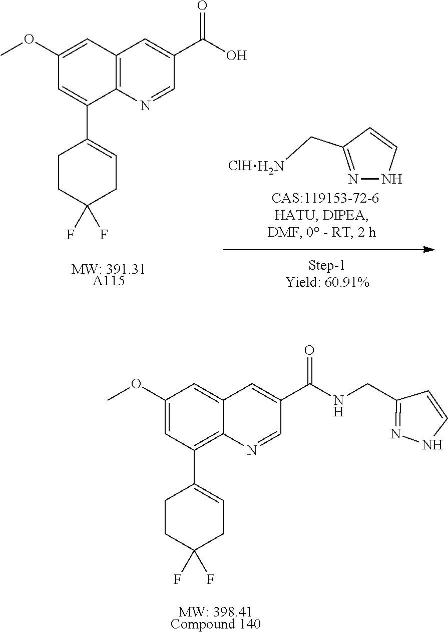

### Step-1

To a stirred solution of A115 (0.15 g, 0.383 mmol, 1.0 eq.) in DMF (2 mL) was added HATU (0.291 g, 0.766 mmol, 2.0 eq.) and DIPEA (0.148 g, 1.14 mmol, 3.0 eq.) respectively at 0° C. Followed by addition of CAS No: 1196153-72-6 (1H-Pyrazol-3-yl) methanamine hydrochloride) (0.076 g, 0.574 mmol, 1.5 eq.) and was maintained for 2 h at RT. TLC indicated the completion of reaction; the resulting reaction mixture was diluted with water (25 mL) and extracted with ethyl acetate (3×50 mL), combined organic layers were dried over Na₂SO₄ and concentrated under reduced pressure to afford the crude. The crude was purified by flash column chromatography by eluting with 50% Ethyl Acetate in Hexane to afford N-((1H-pyrazol-3-yl)methyl)-8-(4, 4-difluorocyclohex-1-en-1-yl)-6-methoxyquinoline-3-carboxamide (Compound 140) (0.114 g, 0.286 mmol, Yield: 60.91%).

¹H NMR (DMSO-d₆, 400 MHz): δ ppm, 12.63 (br s, 1H), 9.21 (br s, 1H), 9.11 (d, J=2.0 Hz, 1H), 8.71 (d, J=2.4 Hz, 1H), 7.65 (d, J=6.8 Hz, 1H), 7.40 (d, J=2.8 Hz, 1H), 7.26 (d, J=3.2 Hz, 1H), 6.22 (d, J=1.6 Hz, 1H), 5.77 (d, J=6.8 Hz, 1H), 4.53 (d, J=3.6 Hz, 2H), 3.91 (s, 3H), 2.86-2.83 (m, 2H) 2.74 (t, J=1.6 Hz, 2H) 2.26-2.15 (m, 2H).

¹H NMR (MeOD, 400 MHz): δ ppm, 9.10 (d, J=2.4 Hz, 1H), 8.67 (d, J=2.4 Hz, 1H), 9.21 (br. s, 1H), 7.32-7.29 (m, 2H), 6.37 (d, J=2.0 Hz, 1H), 5.75 (br. s, 1H), 4.70 (s, 2H), 3.97 (s, 3H), 2.86-2.83 (m, 2H), 2.80-2.73 (m, 2H), 2.33-2.22 (m, 2H). Note: —CONH and —NH proton exchanged in MeOD NMR.

LCMS (Method-A): 2.04 min, 96.98%, 254 nm, MS: ES+399 (M), (M+1)

HPLC (Method-A): 7.16 min, 99.27%, 254 nm,

### Example 132—Synthesis of (R)-8-(4, 4-difluorocyclohex-1-en-1-yl)-6-methoxy-N-(1-(oxazol-2-yl)ethyl)quinoline-3-carboxamide (Compound 141)

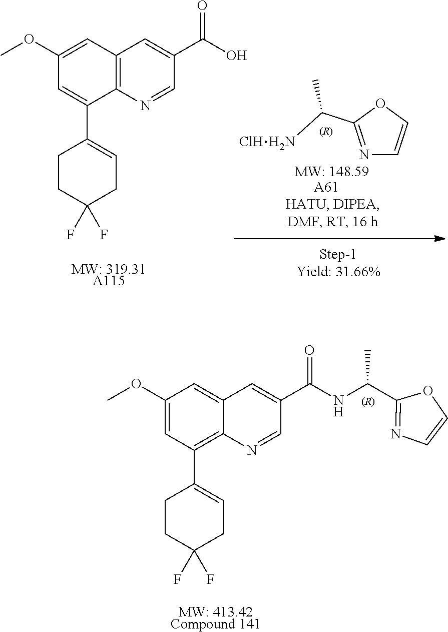

### Step-1

To a stirred solution of A115 (0.1 g, 0.313 mmol, 1.0 eq.), in DMF (1.0 mL) was added HATU (0.178 g, 0.469 mmol, 1.5 eq.), at 0° C. under the N₂ atmosphere. The resulting mixture was stirred at 0° C. for 15 min. then DIPEA (0.121 g, 0.939 mmol, 3.0 eq.), was added and stirred at RT for 15 min. After 15 minutes A61 (0.069 g, 0.469 mmol, 1.5 eq.) was added and resulting mixture was allowed to stirred at RT for 16 h. TLC indicated the completion of reaction; water (3 mL) was added and extracted with ethyl acetate (2×10 mL). The combined organic layers dried over Na₂SO₄ and concentrated under reduced pressure to afford crude. The crude was purified by manual column chromatography using silica (100-200 mesh) as stationary phase (35% Ethyl acetate in Hexane) to afford (R)-8-(4, 4-difluorocyclohex-1-en-1-yl)-6-methoxy-N-(1-(oxazol-2-yl)ethyl)quinoline-3-carboxamide (Compound 141) (0.041 g, 0.099 mmol, Yield 31.66%)

¹H NMR (DMSO, 400 MHz): δ ppm 9.31 (d, J=7.6 Hz, 1H), 9.11 (d, J=1.6 Hz, 1H), 8.73 (d, J=1.6 Hz, 1H), 8.08 (br. s, 1H), 7.42 (d, J=2.4 Hz, 1H), 7.28 (d, J=2.4 Hz, 1H), 7.19 (s, 1H), 5.79 (br. s, 1H), 5.40-5.33 (m, 1H), 3.91 (s, 3H), 2.88 (br. s, 2H), 2.82-2.74 (m, 2H), 2.24-2.17 (m, 2H), 1.60 (d, J=6.8 Hz, 3H).

LCMS (Method A): 2.093 min, 95.44%, 254.0 nm, MS: ES+414.4 (M+1)

HPLC (Method A): 7.94 min, 98.97%, 254 nm

Chiral HPLC: 8.25 min, 98.63%, 254 nm

### Example 133—Synthesis of 8-(4,4-difluorocyclohex-1-en-1-yl)-N-(3-(methyl sulfonamido)propyl)quinoline-3-carboxamide (Compound 142)

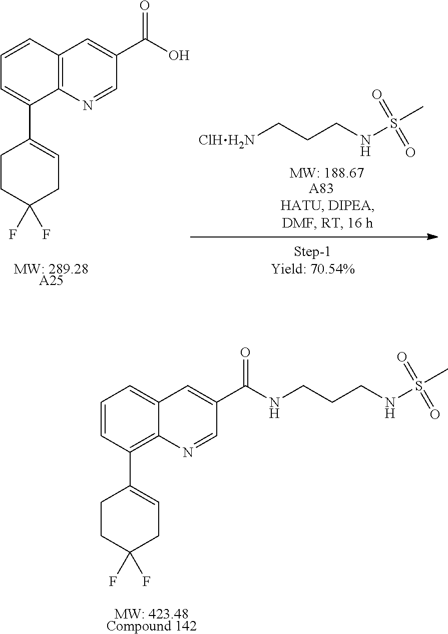

### Step-1

To a stirred solution of, A25 (0.1 g, 0.346 mmol, 1.0 eq.), in DMF in a 10 mL glass vial, was added DIPEA (0.15 mL, 1.035 mmol, 3.0 eq.) and HATU (0.197 g, 0.517 mmol, 1.5 eq.) at 0° C. Followed by addition of (A83) N-(3-aminopropyl) methanesulfonamide hydrochloride (0.078 g, 0.414 mmol, 1.2 equiv). The resulting reaction mixture was stirred at RT for 16 h. TLC indicated the completion of reaction; the resulting reaction mixture was diluted with cold water (30 mL) and extracted with EtOAc (30 mL) three times, organic layer was dried over Na₂SO₄ and evaporated under vacuum. Crude was purified by flash column chromatography on silica gel (100-200 mesh) (desired product eluted in 25% EtOAc in hexane) to afford 8-(4,4-difluorocyclohex-1-en-1-yl)-N-(3-(methylsulfonamido)propyl)quinoline-3-carboxamide (Compound 142) (0.103 g, 0.229 mmol, Yield: 74.54%)

¹H NMR (DMSO-d₆, 400 MHz): δ ppm 9.26 (d, J=2 Hz, 1H), 8.79 (d, J=1.6 Hz, 2H), 8.03 (dd, J=4 Hz, J=7.2 Hz, 1H), 7.67-7.62 (m, 2H), 7.04 (t, J=6 Hz, 1H), 5.76 (br s, 1H), 3.44-3.39 (m, 2H), 3.04 (q, J=6.8 Hz, 2H), 2.91 (s, 3H), 2.83-2.67 (m, 2H), 2.27-2.17 (m, 2H), 1.81-1.74 (m, 2H), 1.81-1.74 (m, 2H).

¹H NMR (MeOD, 400 MHz); δ ppm 9.26 (d, J=2 Hz, 1H), 8.76 (d, J=1.6 Hz, 1H), 7.98 (dd, J=4 Hz, J=7.2 Hz, 1H), 7.70-7.63 (m, 2H), 5.75 (br s, 1H), 3.57 (t, J=6.8 Hz, 2H), 3.21 (t, J=8.5 Hz, 2H), 3.01 (s, 3H), 2.87 (m, J=12.8 Hz, 2H), 2.78 (t, J=14.8 Hz, 2H), 2.34-2.25 (m, 2H). 1.96-1.89 (m, 2H).

LCMS (Method A): 2.065 min, 95.39%, 254.0 nm MS: ES+424.68 [M+H]

HPLC (Method A): 7.09 min, 95.12%, 254.0 nm

### Example 134—Synthesis of (R)-8-(4,4-dimethylcyclohex-1-en-1-yl)-N-(1-hydroxypropan-2-yl)quinoline-3-carboxamide (Compound 143)

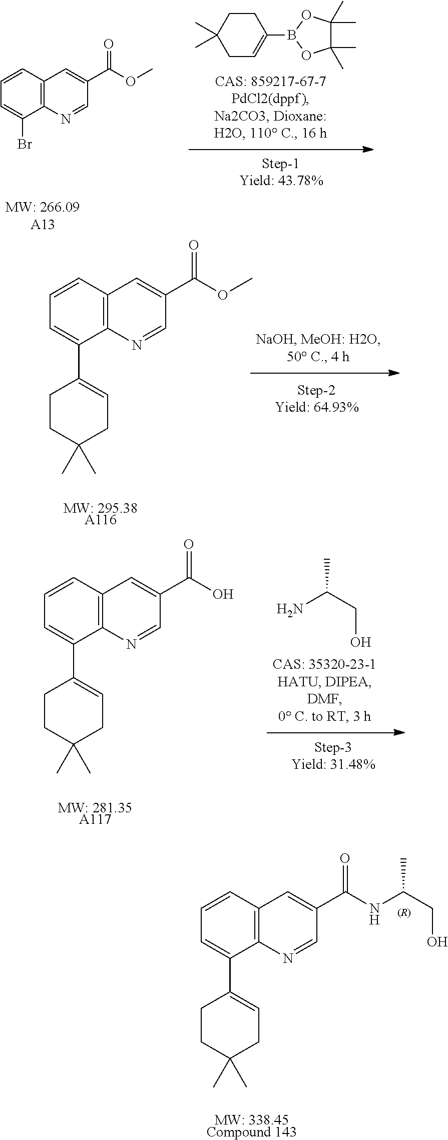

### Step-1

A solution of methyl 8-bromoquinoline-3-carboxylate (A13) (0.5 g, 1.87 mmol, 1.0 eq.), 2-(4, 4-dimethylcyclohex-1-en-1-yl)-4,4, 5, 5-tetramethyl-1, 3, 2-dioxaborolane (CAS: 859217-67-7) (purchased from combi block) (0.44 g, 1.87 mmol, 1.0 eq.) and Na₂CO₃ (0.594 g, 5.61 mmol, 3.0 eq.) in Dioxane:water (4:1) was prepared in 30 mL of glass vial at room temperature under N₂ atmosphere. The reaction solution was degassed by N₂ for 15 minutes at room temperature. To this reaction solution, PdCl₂dppf (0.136 g, 0.187 mmol, 0.1 eq.) was added at same temperature. The glass vial was sealed with cap and heated to 110° C. for 16 h. TLC indicated the completion of reaction; the resulting reaction mixture was cooled to room temperature, diluted with EtOAc (10 mL), filtered it through celite bed, washed it with EtOAc (30 mL) and filtrate was concentrated under reduced pressure to afford crude product. The obtained crude material was purified by flash column chromatography using silica (230-400 Mesh) as a stationary phase (desired product eluted in hexane) to afford methyl 8-(4, 4-dimethylcyclohex-1-en-1-yl)quinoline-3-carboxylate (A116) (0.243 g, 0.822 mmol, Yield: 43.78%).

¹H NMR (DMSO-d₆, 400 MHz): δ ppm, 9.29 (d, J=2.0 Hz, 1H), 8.99 (d, J=2.0 Hz, 1H), 8.10 (dd, J=7.2 Hz, 2.0 Hz, 1H), 7.68-7.62 (m, 2H), 5.74 (br s, 1H), 3.95 (s, 3H), 2.60 (d, J=1.6 Hz, 2H), 2.01 (br s, 2H), 1.52 (t, J=6.4 Hz, 2H), 1.04 (s, 6H).

LCMS (Method A): 2.174 min, 98.68%, 254 nm, MS: ES+296.2 (M+1)

### Step-2

A stirred solution of methyl 8-(4, 4-dimethylcyclohex-1-en-1-yl)quinoline-3-carboxylate (A116) (0.243 g, 0.823 mmol, 1.0 eq.) in MeOH:water (7:3) was prepared in 30 mL of glass vial at room temperature. To this reaction solution, NaOH (0.065 g, 1.647 mmol, 2.0 eq.) was added at same temperature. The glass vial was sealed with cap and heated to 50° C. for 4 h. TLC indicated the completion of reaction; the resulting reaction mixture was directly concentrated under reduced pressure and acidified by using aq. solution of citric acid until pH became acidic to yield precipitate. The precipitate was filtered through Buchner funnel, washed with water (10 mL) and dried under vacuum to afford 8-(4, 4-dimethylcyclohex-1-en-1-yl)quinoline-3-carboxylic acid (A117) (0.150 g, 0.533 mmol, Yield: 64.913).

¹H NMR (DMSO-d₆, 400 MHz): δ ppm 13.49 (br s, 1H), 9.28 (d, J=2.4 Hz, 1H), 8.93 (d, J=2.0 Hz, 1H), 8.07 (dd, J=2.4 Hz & 7.2 Hz, 1H), 7.76-7.61 (m, 2H), 5.74 (br s, 1H), 2.60 (br s, 2H), 2.01 (br s, 2H), 1.52 (t, J=6.4 Hz, 2H), 1.04 (s, 6H).

LCMS (Method A): 2.734 min, 100%, 254 nm, MS: ES+282.2 (M+1)

### Step 3

A stirred solution of 8-(4, 4-dimethylcyclohex-1-en-1-yl)quinoline-3-carboxylic acid (A117) (0.09 g, 0.319 mmol, 1.0 eq.) in DMF (1.0 mL) was prepared in 10 mL of glass vial at room temperature. To this reaction solution, DIPEA (0.16 mL, 0.959 mmol, 3.0 eq.) and HATU (0.182 g, 0.479 mmol, 1.5 eq.) were added at same temperature under nitrogen atmosphere. After 10 min of stirring, (R)-2-Aminopropan-1-ol, CAS: 35320-23-1 (0.024 g, 0.319 mmol, 1.0 eq.) was added at 0° C. Then the resulting reaction mixture was stirred at RT for 3 h. TLC indicated the completion of reaction; the resulting reaction mixture was poured into cold water (15 mL) and extracted with ethyl acetate (20×2), and the combined organic layers dried over Na₂SO₄ and concentrated under reduced pressure to obtain crude product which was purified by normal phase column chromatography using hexane and ethyl acetate (desired product was eluted at 48% ethyl acetate in hexane) further purified by preparative TLC (100% EtOAc as mobile phase) to afford (R)-8-(4, 4-dimethylcyclohex-1-en-1-yl)-N-(1-hydroxypropan-2-yl)quinoline-3-carboxamide (Compound 143) (0.034 g, 0.100 mmol, Yield: 31.48%).

¹H NMR (DMSO-d₆, 400 MHz): δ ppm, 9.26 (d, J=2.0 Hz, 1H), 8.78 (d, J=2.0 Hz, 1H), 8.46 (d, J=7.6 Hz, 1H), 7.98-7.95 (m, 1H) 7.61-760 (m, 2H), 5.74 (br s, 1H), 4.79 (t, J=5.6 Hz, 1H), 4.09-4.06 (m, 1H), 3.53-3.47 (m, 1H), 3.41-3.35 (m, 1H), 2.61 (br s, 2H), 2.01 (br s, 2H), 1.52 (t, J=6.0 Hz, 2H), 1.17 (d, J=6.8 Hz, 3H), 1.05 (s, 6H).

LCMS (Method A): 2.411 min, 100%, 254 nm, MS: ES+339.16 (M+1)

HPLC (Method A): 8.81 min, 98.61%, 210 nm

### Example 135—Synthesis of (R)-8-(4,4-dimethylcyclohex-1-en-1-yl)-N-(1-(oxazol-2-yl)ethyl)quinoline-3-carboxamide (Compound 144)

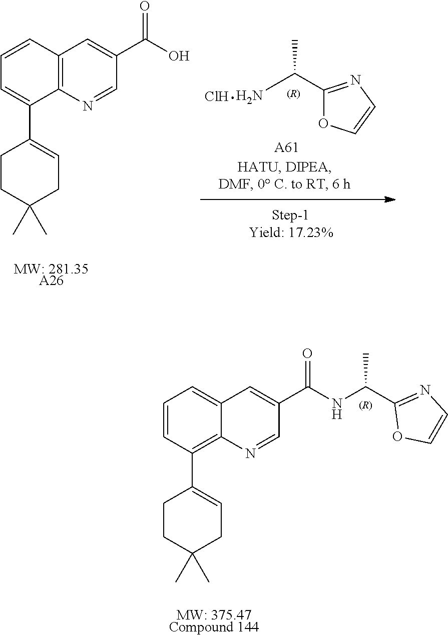

### Step-1

To a stirred solution of 8-(4, 4-dimethylcyclohex-1-en-1-yl)quinoline-3-carboxylic acid (A26) (0.1 g, 0.355 mmol, 1.0 eq.) in DMF (1.0 mL) was added HATU (0.270 g, 0.710 mmol, 2.0 eq.) and DIPEA (0.18 mL, 1.066 mmol, 3.0 eq.) at 0° C. After 10 min of stirring, (R)-1-(oxazol-2-yl)ethan-1-amine hydrochloride (A61) (0.048 g, 0.426 mmol, 1.2 eq.) was added at 0° C. and the resulting reaction mixture was stirred at RT for 6 h. TLC indicated the completion of reaction; the resulting reaction mixture was poured in cold water (5 mL) to obtain precipitate. The precipitate was filtered through Buchner funnel and washed with water (20 mL) and dried under reduced pressure then purified by column chromatography and eluted with 0-2% MeOH in DCM to afford (R)-8-(4, 4-dimethylcyclohex-1-en-1-yl)-N-(1-(oxazol-2-yl)ethyl)quinoline-3-carboxamide (Compound 144) (0.023 g, 0.061 mmol, Yield: 17.23%).

¹H NMR (DMSO-d₆, 400 MHz): δ ppm, 9.33 (d, J=8.0 Hz, 1H), 9.28 (d, J=2.4 Hz, 1H), 8.84 (d, J=2.0 Hz, 1H), 8.08 (s, 1H), 7.98 (t, J=4.8 Hz, 1H), 7.62 (d, J=4.8 Hz, 2H), 7.18 (s, 1H), 5.75 (br s, 1H), 5.39-5.36 (m, 1H), 2.43-2.40 (m, 2H), 2.016 (br s, 2H), 1.60 (d, J=6.8 Hz, 3H), 1.54 (t, J=6.4 Hz, 2H), 1.05 (s, 6H).

LCMS (Method A): 2.694 min, 95.96%, 254 nm, MS: ES+376.22 (M+1)

HPLC (Method A): 9.80 min, 96.80%, 254 nm

Chiral HPLC: 7.72 min, 97.48%, 240 nm

### Example 136—Synthesis of(S)-8-(4,4-dimethylcyclohex-1-en-1-yl)-N-(1-(oxazol-2-yl)ethyl)quinoline-3-carboxamide (Compound 145)

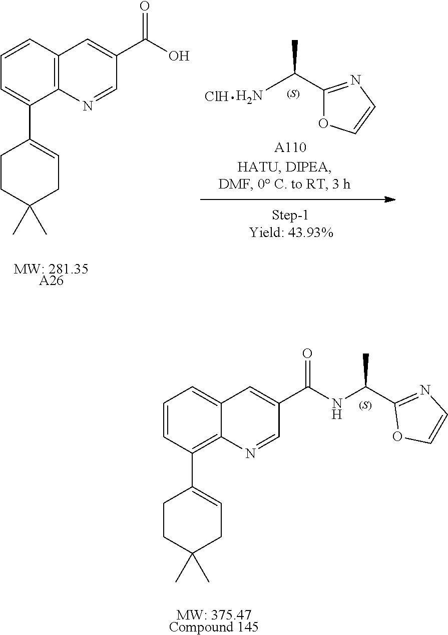

### Step-1

A stirred solution of 8-(4, 4-dimethylcyclohex-1-en-1-yl)quinoline-3-carboxylic acid (A26) (0.05 g, 0.177 mmol, 1.0 eq.) in DMF (1.0 mL) was prepared in 10 mL of glass vial at room temperature. To this reaction solution, DIPEA (0.15 mL, 0.884 mmol, 5.0 eq.) and HATU (0.101 g, 0.266 mmol, 1.5 eq.) were added at same temperature under nitrogen atmosphere. After 10 min of stirring, A110 (0.026 g, 0.177 mmol, 1.0 eq.) was added at 0° C. Then the resulting reaction mixture was stirred at RT for 3 h. TLC indicated the completion of reaction; the resulting reaction mixture was poured into cold water (25 mL) and extracted with ethyl acetate (30×2), and combined organics were dried over Na₂SO₄ and filtered, then filtrate was concentrated under reduce pressure to obtain crude product which was purified by normal phase column chromatography (Desired product was eluted at 40% ethyl acetate in hexane) to afford(S)-8-(4,4-dimethylcyclohex-1-en-1-yl)-N-(1-(oxazol-2-yl)ethyl)quinoline-3-carboxamide (Compound 145) (0.029 g, 0.077 mmol, Yield: 43.93%).

¹H NMR (DMSO-d₆, 400 MHz): δ ppm, 9.34 (d, J=7.6 Hz, 1H), 9.28 (d, J=2.0 Hz, 1H), 8.84 (d, J=2.0 Hz, 1H), 8.08 (s, 1H) 7.98 (t, J=4.8 Hz, 1H), 7.62 (d, J=4.8 Hz, 2H), 7.19 (s, 1H), 5.74 (s, 1H), 5.37-5.34 (m, 1H), 2.61-2.50 (m, 2H), 2.08 (br s, 2H), 1.70 (d, J=6.8 Hz, 3H), 1.52 (t, J=6.0 Hz, 2H), 1.04 (s, 6H).

LCMS (Method A): 2.679 min, 100%, 254 nm, MS: ES+376.17 (M+1)

HPLC (Method A): 9.780 min, 98.73%, 210 nm

### Example 137—Synthesis of N-((1H-pyrazol-5-yl)methyl)-8-(4,4-dimethylcyclohex-1-en-1-yl)quinoline-3-carboxamide (Compound 146)

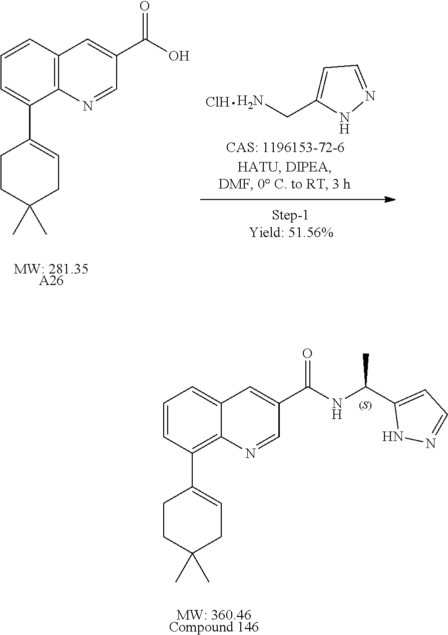

### Step-1

A stirred solution of 8-(4, 4-dimethylcyclohex-1-en-1-yl)quinoline-3-carboxylic acid (A26) (0.05 g, 0.17 mmol, 1.0 eq.) in DMF (1.0 mL) was prepared in 10 mL of glass vial at room temperature. To this reaction solution, DIPEA (0.15 mL, 0.88 mmol, 5.0 eq.) and HATU (0.10 g, 0.26 mmol, 1.5 eq.) were added under nitrogen atmosphere. After 30 min of stirring at 0° C., (1H-pyrazol-5-yl) methenamine hydrochloride (CAS: 1196153-72-6) (0.023 g, 0.17 mmol, 1.0 eq.) was added at same temperature. Then the resulting reaction mixture was stirred from 0° C. to RT for 3 h. TLC indicated the completion of reaction; the resulting reaction mixture was diluted with cold water (5 mL) and extracted with EtOAc (3×5 mL). The combined organic layers were dried over Na₂SO₄, filtered and concentrated under reduced pressure to afford crude. The obtained crude material was purified by flash column chromatography using silica (230-400 mesh) as a stationary phase (desired product eluted in 2% MeOH in DCM) to afford N-((1H-pyrazol-5-yl)methyl)-8-(4,4-dimethylcyclohex-1-en-1-yl)quinoline-3-carboxamide (Compound 146) (0.033 g, 0.09 mmol, Yield: 51.56%).

¹H NMR (DMSO-d₆, 400 MHz): δ ppm, 12.63 (s, 1H), 9.28-9.23 (m, 2H two signals merged together), 8.82 (d, J=2.4 Hz, 1H), 7.96 (t, J=4.8 Hz, 1H), 7.66-7.60 (m, 3H), 6.23 (s, 1H), 5.74 (br s, 1H), 4.54 (br s, 1H), 2.61 (br s, 2H), 2.01 (br s, 2H), 1.52 (t, J=6.4 Hz, 2H), 1.04 (s, 6H).

VT ¹H NMR (DMSO-d₆, 400 MHz): δ ppm, 12.45 (s, 1H), 9.28 (d, J=1.6 Hz, 1H), 8.97 (br s, 1H), 8.78 (d, J=1.6 Hz, 1H), 7.94-7.92 (m, 1H), 7.61-7.58 (m, 3H), 6.24 (s, 1H), 5.77 (s, 1H), 4.57 (d, J=4.4 Hz, 2H), 2.64-2.59 (br s, 2H), 2.04 (br s, 1H), 1.56 (t, J=6.4 Hz, 2H), 1.07 (s, 6H).

LCMS (Method A): 2.450 min, 100%, 254 nm, MS: ES+361.2 (M+1)

HPLC (Method A): 8.80 min, 100%, 254 nm

### Example 138—Synthesis of 8-(4,4-dimethylcyclohex-1-en-1-yl)-N-((4-methyl-1H-pyrazol-5-yl)methyl)quinoline-3-carboxamide (Compound 147)

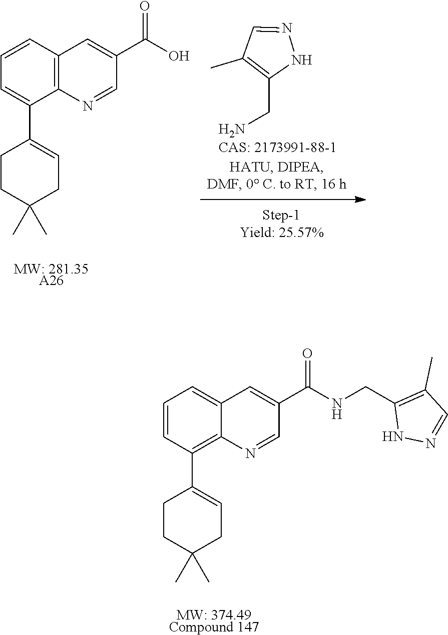

### Step-1

A stirred solution of 8-(4, 4-dimethylcyclohex-1-en-1-yl)quinoline-3-carboxylic acid (A26) (0.05 g, 0.177 mmol, 1.0 eq.) in DMF (0.5 mL) was prepared in 10 mL of glass vial at room temperature. To this reaction solution, DIPEA (0.09 mL, 0.533 mmol, 3.0 eq.) and HATU (0.101 g, 0.266 mmol, 1.5 eq.) were added at same temperature under nitrogen atmosphere. After 10 min of stirring, CAS: 2173991-88-1 (4-Methyl-1H-pyrazol-3-yl) methanamine dihydrochloride, (0.032 g, 0.177 mmol, 1.0 eq.) was added at 0° C. The resulting reaction mixture was stirred at RT for 16 h. TLC indicated the completion of reaction; the resulting reaction mixture was poured into cold water (15 mL) to obtain precipitate. The precipitate was filtered through Büchner funnel and washed with water (20 mL) and dried over high vacuum then crude obtained was purified by preparative TLC (100% EtOAc as mobile phase) to afford 8-(4, 4-dimethylcyclohex-1-en-1-yl)-N-((4-methyl-1H-pyrazol-5-yl)methyl)quinoline-3-carboxamide (Compound 147) (0.017 g, 0.045 mmol, Yield: 25.54%)

¹H NMR (DMSO-d₆, 400 MHz): δ ppm, 12.38 (s, 1H), 9.27 (d, J=2.0 Hz, 1H), 9.09 (br s, 1H), 8.81 (d, J=2.0 Hz, 1H), 7.95 (t, J=4.8 Hz, 1H) 7.60 (d, J=7.6 Hz, 2H), 7.44 (br s, 1H), 5.92-5.73 (m, 1H), 4.60-4.51 (m, 2H), 2.02 (s, 5H), 1.52 (t, J=6.0 Hz, 2H), 1.04 (s, 6H). (Note: 2H merged with DMSO solvent peak).

¹H NMR (MeOD, 400 MHz): δ ppm, 9.25 (d, J=2.0 Hz, 1H), 8.74 (s, 1H), 7.92-7.90 (m, 1H), 7.62-7.59 (m, 2H) 7.43 (br s, 1H), 5.77 (br s, 1H), 4.67 (br s, 2H), 2.61 (br s, 2H), 2.14 (s, 3H), 2.08 (br s, 2H), 1.63 (t, J=6.4 Hz, 2H), 1.09 (s, 6H). Note: —CONH and —NH proton exchanged in MeOD NMR.

LCMS (Method A): 2.540 min, 99.36%, 254 nm, MS: ES+375.17 (M+1)

HPLC (Method A): 9.20 min, 98.70%, 210 nm

### Example 139—Synthesis of(S)-8-(4,4-dimethylcyclohex-1-en-1-yl)-N-(1-methoxypropan-2-yl)quinoline-3-carboxamide (Compound 148)

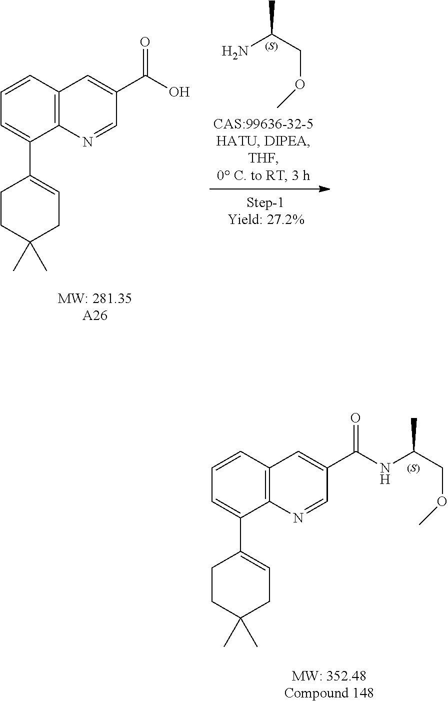

### Step-1

A stirred solution of 8-(4, 4-dimethylcyclohex-1-en-1-yl)quinoline-3-carboxylic acid (A26) (0.1 g, 0.35 mmol, 1 eq.) in THF (1 mL) were prepared in 10 mL glass vial at room temperature. To this reaction solution, DIPEA (0.13 g, 1.06 mmol, 3 eq.) was added at same temperature followed by addition of HATU (0.19 g, 0.52 mmol, 1.5 eq.) at 0° C. and reaction mixture stirred for 10 min. Then (CAS: 99636-32-5) (S)-(+)-1-methoxy-2-propylamine (0.03 g, 0.35 mmol, 1 eq.) was added and resulting reaction mixture was stirred at RT for 16 h. TLC indicated the completion of reaction; the resulting reaction mixture was extracted by EtOAc (20 mL×3), combined organic layer was dried over Na₂SO₄, concentrated under reduced pressure to afford the crude. The obtained crude was purified by flash column chromatography using silica gel (230-400 mesh). (Eluent gradient 10% EtOAc:Hexane) to afford(S)-8-(4, 4-dimethylcyclohex-1-en-1-yl)-N-(1-methoxypropan-2-yl)quinoline-3-carboxamide (Compound 148) (0.034, 0.096 mmol, Yield: 27.2%).

¹H NMR (DMSO-d₆, 400 MHz): δ ppm, 9.25 (d, J=2 Hz, 1H), 8.77 (d, J=2 Hz, 1H), 8.59 (d, J=4 Hz, 1H), 7.99-7.96 (m, 1H), 7.63-7.60 (m, 2H), 5.74 (br s, 1H), 4.29-4.22 (m, 1H), 3.47-3.43 (m, 1H), 3.35-3.30 (m, 1H), 3.29 (s, 3H), 2.68-2.33 (m, 2H), 2.01 (br s, 2H), 1.52 (t, J=6.4 Hz, 2H), 1.18 (d, J=2.8 Hz, 3H), 1.10 (s, 6H).

¹H NMR (MeOD, 400 MHz): δ ppm, 9.22 (s, 1H), 8.72 (s, 1H), 7.93-7.91 (m, 1H), 7.63-7.59 (m, 2H), 5.78 (br s, 1H), 4.43-4.38 (m, 1H), 3.57-3.53 (m, 1H), 3.50-3.46 (m, 1H), 3.36 (s, 3H), 2.63-2.61 (m, 2H), 2.08 (br s, 2H), 1.64 (t, J=6.4 Hz, 2H), 1.30 (d, J=6.8 Hz, 3H), 0.92 (s, 6H). Note: —CONH proton exchanged in MeOD NMR.

LCMS (Method A): 2.735 min, 100%, 254 nm, MS: ES+275M+1)

HPLC (Method A): 10.22 min, 100%, 254 nm

### Example 140—Synthesis of (R)-8-(4,4-dimethylcyclohex-1-en-1-yl)-N-(4-(methylamino)-4-oxobutan-2-yl)quinoline-3-carboxamide (Compound 149)

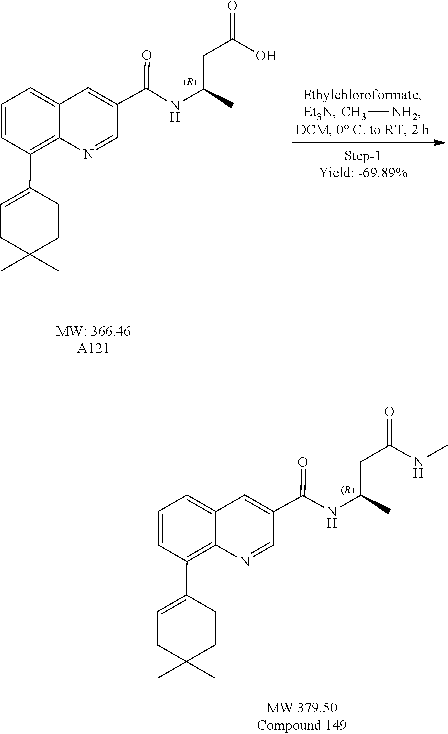

### Step-1

A stirred solution of A121 (0.09 g, 0.245 mmol, 1.0 eq.) in DCM (1.0 mL) was prepared in 10 mL glass vial at 0° C. To this reaction solution was added TEA (0.1 mL, 0.368 mmol, 1.5 eq.), Ethylchloroformate (0.040 g, 0.368 mmol, 1.5 eq.) at 0° C. and stirred for 1 h. followed by addition of methyl amine (0.18 mL, 0.368 mmol, 1.5 eq.) at same temperature and resulting reaction mixture was again stirred at RT for 2 h. TLC indicated the completion of reaction. The resulting reaction mixture was diluted with water (30 mL) and extracted with EtOAc (2×20 mL). The combined organic layer was dried over Na₂SO₄, filtered and concentrated under high vacuum to obtain crude product. The crude product was purified by combi-flash column chromatography using 230-400 mesh size silica and product was eluted at 25% EtOAc:Hexane to afford as a (R)-8-(4, 4-dimethylcyclohex-1-en-1-yl)-N-(4-(methylamino)-4-oxobutan-2-yl)quinoline-3-carboxamide (Compound 149) (0.065 g, 0.171 mmol, Yield: 69.89%).

¹H NMR (DMSO-d₆, 400 MHz): δ ppm, 9.23 (d, J=1.6 Hz, 1H), 8.74 (d, J=2.0 Hz, 1H), 8.61 (d, J=7.6 Hz, 1H), 7.98-7.96 (m, 1H), 7.86-7.85 (d, J=4.4 Hz, 1H), 7.61-7.60 (m, 1H), 5.74 (br s, 1H), 4.40-4.37 (m, 1H), 2.67-2.57 (m, 5H), 2.46-2.42 (m, 1H), 2.32-2.28 (m, 1H), 2.01 (br s, 2H), 1.52 (t, J=6.0 Hz, 2H), 1.19 (d, J=6.4 Hz, 3H), 1.05 (s, 6H).

¹H NMR (MeOD-d₃, 400 MHz): δ ppm, 9.18 (d, J=2.0 Hz, 1H), 8.67 (d, J=2.0 Hz, 1H), 7.99 (br s, 1H), 7.90-7.88 (m, 1H), 7.61-7.58 (m, 2H), 5.76-5.74 (m, 1H), 4.56-4.50 (m, 1H), 3.23-3.17 (m, 1H), 2.72 (d, J=4.4 Hz, 3H), 2.60-2.54 (m, 3H), 2.48-2.43 (m, 1H), 2.05-2.05 (m, 2H), 1.61 (t, J=6.4 Hz, 2H), 1.33-1.28 (m, 3H), 1.05 (s, 6H). Note: —CONH proton exchanged in MeOD NMR.

LCMS (Method A): 2.336 min, 100.00%, 254 nm, MS: ES+380.27 (M+1)

HPLC (Method B): 8.60 min, 100.00%, 254.0 nm

Chiral HPLC (Method B): 9.06 min, 100.00%, 240.0 nm

### Example 141—Synthesis of N-(cyanomethyl)-8-(4,4-dimethylcyclohex-1-en-1-yl)quinoline-3-carboxamide (Compound 150)

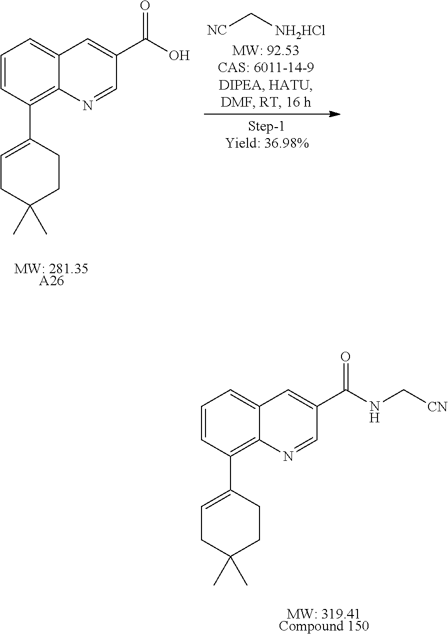

### Step-1

A stirred solution of A26 (0.05 g, 0.177 mmol, 1.0 eq.) in DMF (1 mL) was prepared in 10 mL glass vial at room temperature. To this reaction solution was added DIPEA (0.15 mL, 0.888 mmol, 5.0 eq.) and HATU (0.067 g, 0.177 mmol, 1.0 eq.) at RT and stirred for 10 min. Then addition of 2-aminoacetonitrile hydrochloride (CAS: 6011-14-9) (0.019 g, 0.213 mmol, 1.2 eq.) at same temperature and resulting reaction mixture was stirred at RT for 16 h, TLC indicated the completion of reaction; the resulting reaction mixture was diluted with water (30 mL) and extracted with EtOAc (2×30 mL). The combined organic layer was dried over Na₂SO₄, filtered and concentrated under high vacuum to obtain crude product. The crude product was purified by combi-flash column chromatography using 230-400 mesh size silica and product was eluted at 0-30% EtOAc:Hexane to afford as a N-(cyanomethyl)-8-(4, 4-dimethylcyclohex-1-en-1-yl)quinoline-3-carboxamide (Compound 150) (0.021 g, 0.065 mmol, Yield: 36.98%).

¹H NMR (DMSO-d₆, 400 MHz): δ ppm, 9.55 (t, J=5.2 Hz, 1H), 9.25 (d, J=2.0 Hz, 1H), 8.84 (d, J=2.0 Hz, 1H), 8.00 (t, J=4.8 Hz, 1H), 7.64-7.61 (m, 2H), 5.74 (s, 1H), 4.41 (d, J=5.2 Hz, 2H), 2.61 (br s, 2H), 2.01 (br s, 2H), 1.53 (t, J=6.4 Hz, 2H), 1.05 (s, 6H).

¹H NMR (MeOD, 400 MHz): δ ppm, 9.25 (d, J=2.4 Hz, 1H), 8.77 (d, J=2.4 Hz, 1H), 7.94 (dd, J=2.0 Hz and 5.2 Hz, 2H), 5.78 (s, 1H), 4.44 (s, 2H), 2.62 (d, J=2.0 Hz, 2H), 2.08 (d, J=2.8 Hz, 2H), 1.64 (d, J=6.4 Hz, 2H), 1.10 (s, 6H). Note: —CONH proton exchanged in MeOD NMR.

LCMS (Method A): 2.651 min, 100.0%, 254 nm, MS: ES+320.16 (M+1)

HPLC (Method A): 9.66 min, 97.73%, 254 nm

### Example 142—Synthesis of N-(1-(1H-pyrazol-5-yl)ethyl)-8-(4,4-dimethylcyclohex-1-en-1-yl)quinoline-3-carboxamide (Compound 151)

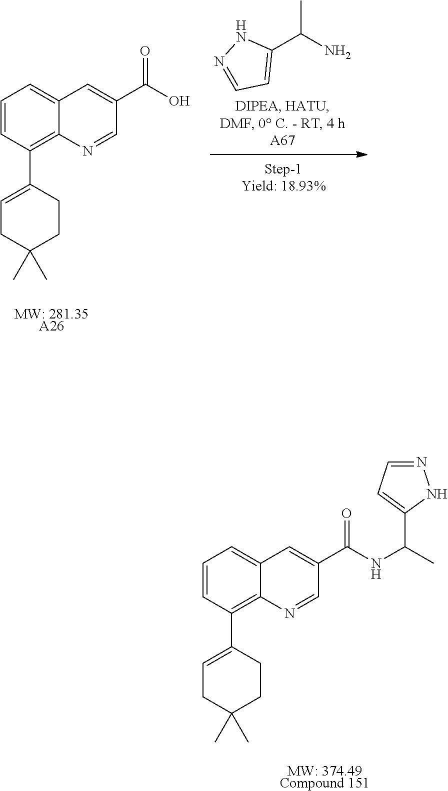

### Step-1

To a stirred solution of 8-(4, 4-dimethylcyclohex-1-en-1-yl)quinoline-3-carboxylic acid (A26) (0.1 g, 0.35 mmol, 1.0 eq.) in DMF (1.0 mL) was added HATU (0.20 g, 0.533 mmol, 1.5 eq.) at 0° C. Then after 10 min at same temperature DIPEA (0.18 mL, 0.106 mmol, 3.0 eq.) was added and stirred for another 10 min. After 10 min of stirring, 1-(1H-pyrazol-5-yl)ethan-1-amine (A67) (CAS: 1179072-43-5) (0.043 g, 0.390 mmol, 1.1 eq.) was added at 0° C. The resulting reaction mixture was stirred at RT for 4 h. TLC indicated the completion of reaction; the resulting reaction mixture was poured in cold water (5 mL) to obtain precipitate. The precipitate was filtered through Buchner funnel and washed with water (20 mL) and dried over high vacuum and purified by column chromatography and eluted with 50% EtOAC in Hexane to afford N-(1-(1H-pyrazol-5-yl)ethyl)-8-(4, 4-dimethylcyclohex-1-en-1-yl)quinoline-3-carboxamide (Compound 151) (0.025 g, 0.066 mmol, Yield: 18.93%).

¹H NMR (DMSO-d₆, 400 MHz): δ ppm, 12.58 (br s, 1H) 9.28 (d, J=2.4, 1H), 9.02 (br s, 1H), 8.82 (d, J=2.0 Hz, 1H), 7.97 (t, J=5.2 Hz, 1H), 7.63-7.60 (m, 3H), 6.25 (s, 1H), 5.74 (br s, 1H), 5.34-5.32 (m, 1H), 2.65-2.60 (m, 2H), 2.02-2.01 (m, 2H), 1.52 (t, J=6.8 Hz, 5H), 1.05 (s, 6H).

LCMS (Method A): 2.486 min, 98.95%, 254 nm, MS: ES+375.27 (M+1)

HPLC (Method A): 9.20 min, 98.06%, 254 nm

### Example 143—Synthesis of (R)—N-(1-cyano-3-(methylamino)-3-oxopropyl)-8-(4, 4-dimethylcyclohex-1-en-1-yl)quinoline-3-carboxamide (Compound 152)

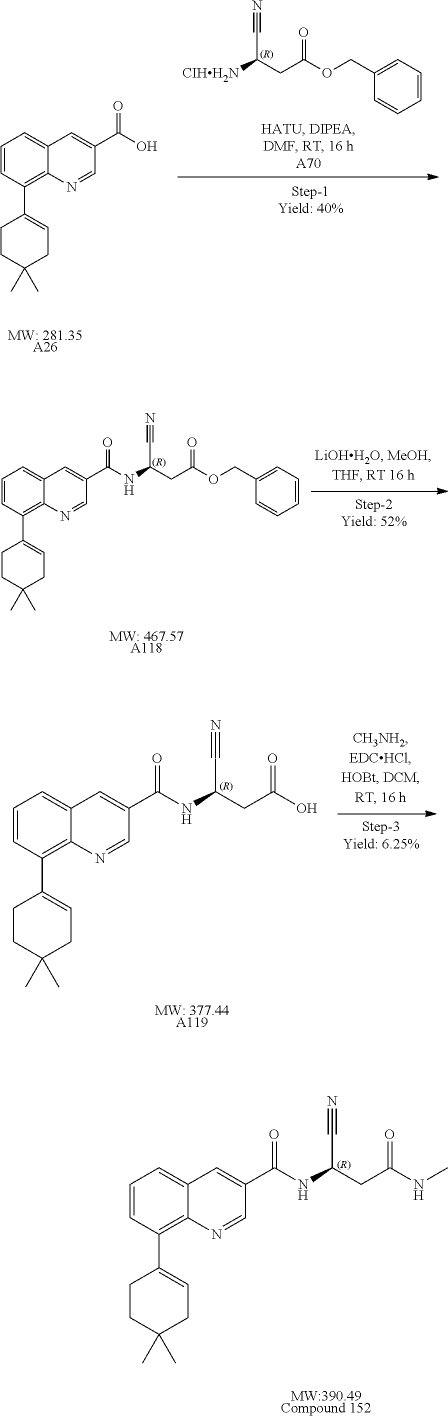

### Step-1

To a stirred solution of A26 (0.49 g, 1.75 mmol, 1.0 eq.) in DMF (5.0 mL) was added HATU (1.0 g, 2.63 mmol, 1.5 eq.) and DIPEA (0.98 g, 5.28 mmol, 3.0 eq.) at RT and stirred for 15 min. After 15 min, addition of A70 (0.55 g, 2.28 mmol, 1.3 eq.) at same temperature. Then the resulting mixture was stirred at room temperature for 16 h. TLC indicated the completion of reaction; the resulting reaction mixture was diluted with water (10 mL) and extracted with EtOAc (3×20 mL). The combined organic layer was dried over anhy. Na₂SO₄, filtered, and concentrated under reduced pressure to afford crude product. The crude product was purified by manual column chromatography (desired product eluted in 3% MDC in MeOH) to afford benzyl(R)-3-cyano-3-(8-(4, 4-dimethylcyclohex-1-en-1-yl)quinoline-3-carboxamido) propanoate (A118) (0.32 g, 0.68 mmol, Yield: 40%)

LCMS: 3.033 min, 40.35%, 220 nm, MS: ES+468.5 (M+1)

### Step-2

To a stirred solution of A118 (0.32 g, 0.68 mmol, 1.0 eq.) in THF:MeOH (3.0 mL, 2:1 vol) was added aq. LiOH (1.0 mL, 1 vol of 1N solution). Then the resulting mixture was stirred at room temperature for 16 h. TLC indicated the completion of reaction; the resulting reaction mixture was diluted with water (10 mL) acidified with 1N HCl and extracted with EtOAc (3×20 mL). The combined organic layer was dried over anhy. Na₂SO₄, filtered, and concentrated under reduced pressure to afford crude product (R)-3-cyano-3-(8-(4, 4-dimethylcyclohex-1-en-1-yl)quinoline-3-carboxamido) propanoic acid (A119) (0.13 g, 0.34 mmol, Yield: 52%)

LCMS: 2.43 min, 23.73%, 254 nm, MS: ES+378.4 (M+1)

### Step-3

To a stirred solution of (A119) (0.12 g, 0.31 mmol, 1.0 eq.) in DCM (2.0 mL) were added EDC·HCl (0.12 g, 4.75 mmol, 2.0 eq.) and HOBt (0.085 g, 6.34 mmol, 2.0 eq.) at RT and stirred for 10 min. After 10 min, addition of CH₃—NH₂ (2 M solution in THF) (0.37 mL, 6.34 mmol, 2 eq.)) at RT and resulting mixture was again stirred at same temperature for 16 h. TLC indicated the completion of reaction; the resulting reaction mixture was diluted with water (10 mL) and extracted with EtOAc (3×20 mL). The combined organic layer was dried over anhy. Na₂SO₄, filtered and concentrated under reduced pressure to afford crude product. The crude product was purified by Prep HPLC purification (Method-A) to afford (R)—N-(1-cyano-3-(methylamino)-3-oxopropyl)-8-(4,4-dimethylcyclohex-1-en-1-yl)quinoline-3-carboxamide (Compound 152) (0.0075 g, 0.019 mmol, Yield: 6.25%). 1H NMR (MeOD, 400 MHz): δ ppm 9.24 (d, J=2.0 Hz, 1H), 8.75 (d, J=2.4 Hz, 1H), 7.94 (dd, J=2.4 Hz & 7.6 Hz, 1H), 7.67-7.63 (m, 2H), 5.78 (s, 1H), 5.14 (t, J=7.2 Hz, 1H), 2.98 (t, J=6.0 Hz, 2H), 2.78 (s, 3H), 2.62 (d, J=1.6 Hz, 2H), 2.08 (d, J=3.2 Hz, 2H), 1.64 (t, J=6.4 Hz, 2H), 1.10 (s, 6H). Note: —CONH proton exchanged in MeOD NMR.

HPLC (Method A): 8.81 min, 97.45%, 254 nm.

LCMS (Method A): 2.511 min, 100%, 254 nm, MS: ES+391.3 (M+1)

### Example 144—Synthesis of (R)—N-(4-amino-4-oxobutan-2-yl)-8-(4, 4-dimethyl cyclohex-1-en-1-yl)quinoline-3-carboxamide (Compound 153)

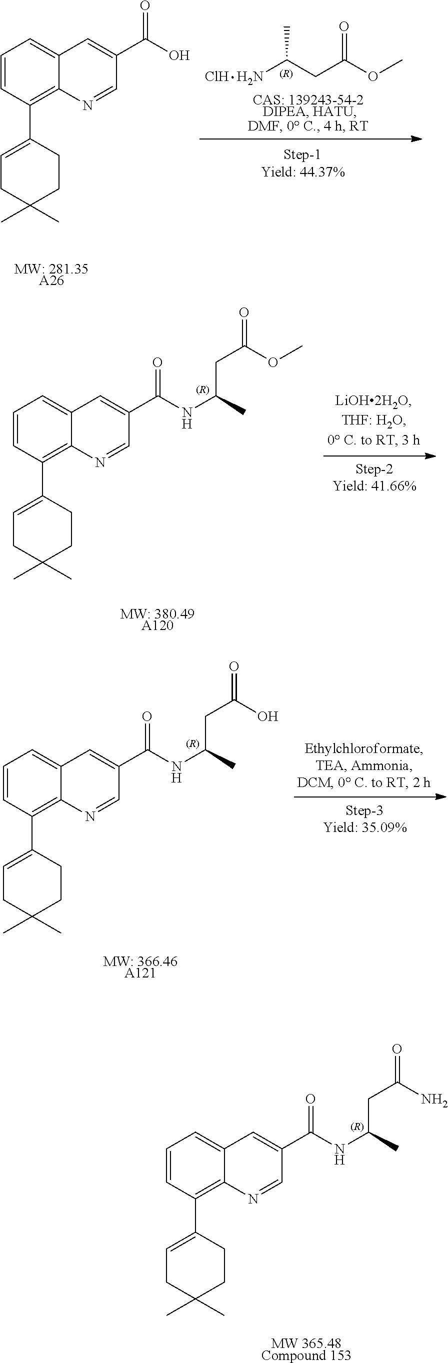

### Step-1

A stirred solution of A26 (0.25 g, 0.888 mmol, 1.0 eq.) in DMF (4.0 mL) was prepared in 30 mL glass vial at room temperature. To this reaction solution were added DIPEA (0.45 mL, 2.665 mmol, 3.0 eq.), HATU (0.36 g, 0.888 mmol, 1.0 eq.) at RT and stirred for 10 min. Then addition of methyl (R)-3-aminobutanoate hydrochloride (CAS: 139243-54-2) (0.20 g, 1.332 mmol, 1.5 eq.) at same temperature and resulting reaction mixture was stirred at 0° C. to RT 4 h. TLC indicated the completion of reaction; the resulting reaction mixture was diluted with water (30 mL) and extracted with EtOAc (2×30 mL). The combined organic layer was dried on Na₂SO₄, filtered and concentrated under high vacuum to obtain crude product. The crude product was purified by combi-flash column chromatography (desired product eluted in 25% EtOAc:Hexane) to afford methyl (R)-3-(8-(4, 4-dimethylcyclohex-1-en-1-yl)quinoline-3-carboxamido) butanoate (A120) (0.15 g, 0.394 mmol, Yield: 44.37%).

¹H NMR (DMSO-d₆, 400 MHz): δ ppm, 9.24 (d, J=2.0 Hz, 1H), 8.74 (d, J=2.0 Hz, 1H), 8.67 (d, J=7.6 Hz, 1H), 8.00-7.97 (m, 1H), 7.63-7.67 (m, 2H), 5.74 (br s, 1H), 4.46-4.39 (m, 1H), 3.60 (s, 3H), 2.71-2.66 (m, 3H), 2.61 (br s, 3H), 2.58-2.56 (m, 3H), 2.01 (br s, 2H), 1.52 (t, J=6.4 Hz, 2H), 1.30-1.15 (m, 3H), 1.05 (s, 6H).

LCMS (Method A): 2.713 min, 94.72%, 254 nm, MS: ES+381.27 (M+1)

### Step-2

A stirred solution of (A120) (0.15 g, 0.394 mmol, 1.0 eq.) in THF:H₂O (1:1, 3.0 Ml, 2 vol) was prepared in 10 Ml glass vial at 0° C. To this reaction solution was added LiOH·2H₂O (0.05 g, 1.182 mmol, 3.0 eq.) and stirred the reaction mixture at RT for 3 h. TLC indicated the completion of reaction; the resulting reaction mixture was diluted with water (10 Ml) and extracted with diethyl ether (2×30 Ml). Then aqueous layer was acidified by aq. citric acid up to Ph ˜4 and again extracted with EtOAc (3×10 Ml). Then combined organic layer (after acidified) was dried over anhy. Na₂SO₄, filtered and concentrated under reduced pressure to afford (R)-3-(8-(4, 4-dimethylcyclohex-1-en-1-yl)quinoline-3-carboxamido)butanoic acid (A121) (0.07 g, 0.191 mmol, Yield: 41.66%).

¹H NMR (DMSO-d₆, 400 MHz): δ ppm, 12.25 (br s, 1H), 9.24 (d, J=2.4 Hz, 1H), 8.74 (d, J=2.0 Hz, 1H), 8.66 (d, J=7.6 Hz, 1H), 7.98-7.96 (m, 1H), 7.65-7.60 (m, 2H), 5.74 (s, 1H), 4.43-4.36 (m, 1H), 3.60 (s, 3H), 2.73-2.61 (m, 2H), 2.01 (br s, 2H), 1.52 (t, J=6.4 Hz, 2H), 1.30-1.15 (m, 3H), 1.24 (s, 6H).

LCMS (Method A): 2.427 min, 99.45%, 254 nm, MS: ES+367.32 (M+1)

### Step-3

A stirred solution of (A121) (0.06 g, 0.163 mmol, 1.0 eq.) in DCM (0.6 mL) was prepared in 10 mL glass vial at 0° C. To this reaction solution were added TEA (0.025 g, 0.245 mmol, 1.5 eq.), Ethylchloroformate (0.027 g, 0.245 mmol, 1.5 eq.) at 0° C. and stirred for 1 h. Then addition of aq. NH₄OH (0.6 g, 10 vol) at same temperature and resulting reaction mixture was again stirred at 0° C. to RT 2 h. TLC indicated the completion of reaction; the resulting reaction mixture was diluted with water (30 mL) and extracted with EtOAc (2×10 mL). The combined organic layer was dried on Na₂SO₄, filtered and concentrated under high vacuum to obtained crude product. The crude product was purified by combi-flash column chromatography (desired product eluted in 25% EtOAc:Hexane) to afford (R)—N-(4-amino-4-oxobutan-2-yl)-8-(4, 4-dimethylcyclohex-1-en-1-yl)quinoline-3-carboxamide (Compound 153) (0.021 g, 0.057 mmol, Yield: 35.09%).

¹H NMR (DMSO-d₆, 400 MHz): δ ppm, 9.24 (d, J=2.4 Hz, 1H), 8.74 (d, J=2.4 Hz, 1H), 8.61 (d, J=8.0 Hz, 1H), 7.97 (dd, J=4.0 Hz & 6.0 Hz, 1H), 7.61-7.60 (m, 2H), 7.38 (br s, 1H), 6.87 (br s, 1H), 5.74 (s, 1H), 4.39 (m, 1H), 2.62-2.61 (m, 2H), 2.47-2.42 (m, 1H), 2.22-2.26 (m, 1H), 2.01 (d, J=3.2 Hz, 2H), 1.52 (t, J=6.8 Hz, 2H), 1.23-1.20 (m, 3H), 1.05 (s, 6H).

¹H NMR (MeOD-d₃, 400 MHz): δ ppm, 9.22 (d, J=2.0 Hz, 1H), 8.71 (d, J=2.4 Hz, 1H), 7.92 (dd, J=2.8 Hz & 6.8 Hz, 1H), 7.64-7.61 (m, 2H), 6.87 (br s, 1H), 5.79-5.77 (m, 1H), 4.60-4.55 (m, 1H), 2.66-2.59 (m, 3H), 2.53-2.48 (m, 1H), 2.08 (d, J=3.6 Hz, 2H), 1.64 (t, J=6.4 Hz, 2H), 1.39-1.37 (m, 3H), 1.10 (s, 6H). Note: —CONH and —CONH2 protons exchanged in MeOD NMR.

LCMS (Method A): 2.273 min, 99.73%, 254 nm, MS: ES+366.4 (M+1)

HPLC (Method A): 8.25 min, 98.94%, 254.0 nm

### Example 145—Synthesis of(S)-8-(4, 4-dimethylcyclohex-1-en-1-yl)-N-(1-hydroxy-3-methoxypropan-2-yl)quinoline-3-carboxamide (Compound 154)

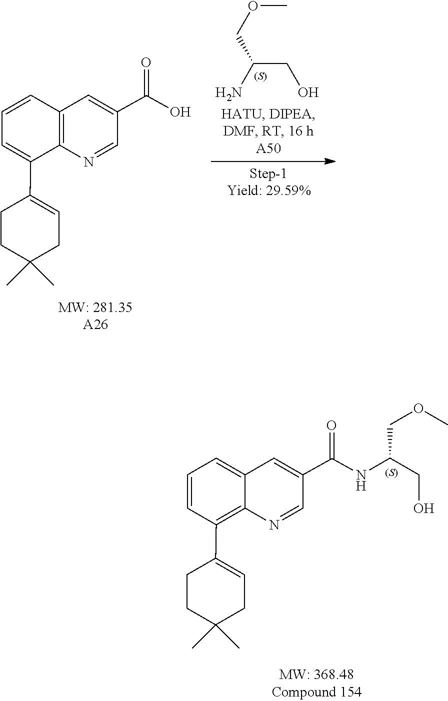

### Step-1

To a solution of 8-(4, 4-dimethylcyclohex-1-en-1-yl)quinoline-3-carboxylic acid (A26) (0.08 g, 0.284 mmol, 1.0 eq.) in DMF (2.0 mL), were added HATU (0.162 g, 0.42 mmol, 1.5 eq.) and DIPEA (0.15 mL, 0.85 mmol, 3.0 eq.) at 0° C. The reaction mixture was stirred under N₂ atmosphere. After 15 min of stirring at 0° C., (S)-2-amino-3-methoxypropan-1-ol (A50) (0.060 g, 0.568 mmol, 2.0 eq.) was added at same temperature and the resulting reaction mixture was stirred at 0° C. to room temperature for 16 h. TLC indicated the completion of reaction; the resulting reaction mixture was quenched with ice-cold water (10 mL) and extracted with ethyl acetate (10 mL×3). The combined organic layers were dried over Na₂SO₄, filtered and concentrated under reduced pressure to afford crude. The crude was purified by column chromatography using silica gel (230-400 mesh) as stationary phase (60% Ethyl acetate in hexane as gradient) to afford(S)-8-(4, 4-dimethylcyclohex-1-en-1-yl)-N-(1-hydroxy-3-methoxypropan-2-yl)quinoline-3-carboxamide (Compound 154) (0.031 g, 0.084 mmol, Yield: 29.59%).

¹H NMR (DMSO-d₆, 400 MHz): δ ppm 9.27 (s, 1H), 8.80 (s, 1H), 8.51 (d, J=7.6 Hz, 1H), 7.98 (s, 1H), 7.62-7.61 (d, J=3.6 Hz, 2H), 5.75 (br s, 1H), 4.83 (s, 1H), 4.21-4.20 (m, 1H), 3.54-3.50 (m, 4H), 3.29 (s, 3H), 2.75-2.62 (m, 2H), 2.02 (s, 2H), 1.53 (s, 2H), 1.05 (s, 6H).

LCMS (Method A): 2.40 min, 96.55%, 254.0 nm, MS: ES+369.4 (M+1)

HPLC (Method A): 8.73 min, 96.49%, 254.0 nm

Chiral HPLC (Method A): 8.73 min, 95.03%, 242.0 nm.

### Example 146—Synthesis of 8-(4,4-dimethylcyclohex-1-en-1-yl)-N-isopropyl-6-methoxyquinoline-3-carboxamide (Compound 155)

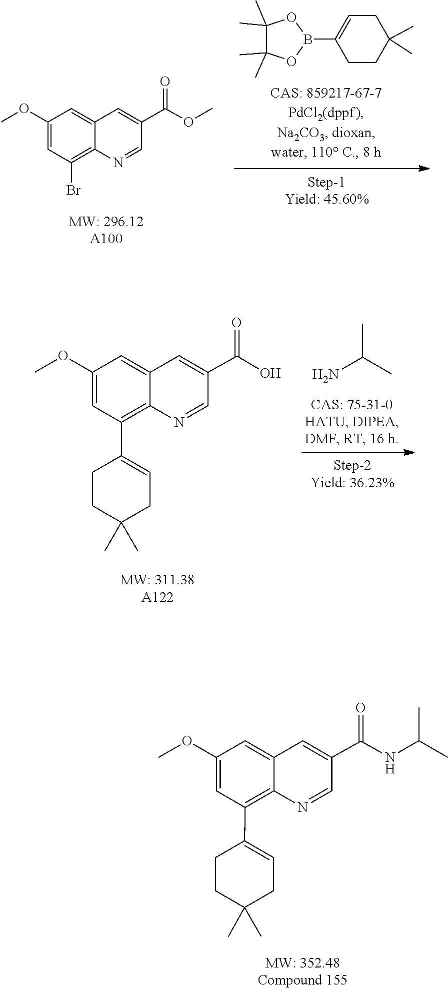

### Step-1

To a stirred solution of 2-(4, 4-dimethylcyclohex-1-en-1-yl)-4,4, 5, 5-tetramethyl-1, 3, 2-dioxaborolane CAS: 859217-67-7 (1.84 g, 8.442 mmol, 1.0 eq.), methyl 8-bromo-6-methoxyquinoline-3-carboxylate (A100) (2.50 g, 8.442 mmol, 1.0 eq.) and Na₂CO₃ (1.77 g, 16.88 mmol, 2.0 eq.) in Dioxane (40 mL) and water (10 mL) was added Pd(dppf)Cl₂·DCM (1.21 g, 1.688 mmol, 0.2 eq.) under N₂. The resulting mixture was stirred at 110° C. for 8 h. TLC indicated the completion of reaction; the resulting reaction mixture was filtered through celite bed and concentrated under reduced pressure to afford crude. The obtained crude material was purified by flash column chromatography using silica gel (230-400 mesh) as stationary phase (85% EtOAc in Hexane) to afford (A122) 8-(4, 4-dimethylcyclohex-1-en-1-yl)-6-methoxyquinoline-3-carboxylic acid (1.2 g, 3.85 mmol, Yield: 45.60%). (Note: -observed one pot coupling and hydrolysis) ¹H NMR (DMSO-d₆, 400 MHz): δ ppm, 13.42 (br s, 1H), 9.11 (d, J=2.4 Hz, 1H), 8.80 (d, J=2.0 Hz, 1H), 7.48 (d, J=7.6 Hz, 1H), 7.23 (d, J=2.8 Hz, 1H), 5.76 (br s, 1H), 3.91 (s, 3H), 2.57 (br s, 2H), 1.98 (br s, 2H), 1.50 (t, J=6.0 Hz, 2H), 1.02 (s, 6H).

LCMS (Method A): 2.57 min, 78%, 254.0 nm, MS: ES+312

### Step-2

To a stirred solution of 8-(4, 4-dimethylcyclohex-1-en-1-yl)-6-methoxyquinoline-3-carboxylic acid (A122) (0.100 g, 0.321 mmol, 1.0 eq.) in DMF (10 mL), was added isopropylamine CAS: 75-31-0 (0.024 g, 0.321 mmol, 1.0 eq.), DIPEA (0.125 g, 0.963 mmol, 3.0 eq.) and HATU (0.182 g, 0.481 mmol, 1.5 eq.) then stirring continued at room temperature for 16 h. TLC indicated the completion of reaction; the resulting reaction mixture was diluted with water (50 mL) and extracted by EtOAc (2×75 mL). Organic phase was dried over Na₂SO₄ and concentrated under vacuum to afford crude. The obtained crude material was purified by flash column chromatography using silica gel (230-400 mesh) as stationary phase (26% EtOAc in Hexane) to afford 8-(4, 4-dimethylcyclohex-1-en-1-yl)-N-isopropyl-6-methoxyquinoline-3-carboxamide (Compound 155) (0.041 g, 0.116 mmol, 36.23%)

¹H NMR (DMSO-d₆, 400 MHz): δ ppm, 9.08 (d, J=2.4 Hz, 1H), 8.64 (d, J=2.0 Hz, 1H), 8.51 (d, J=7.6 Hz, 1H), 7.36 (d, J=2.8 Hz, 1H), 7.19 (d, J=2.8 Hz, 1H), 5.75 (br s, 1H), 4.17-4.12 (m, 1H), 3.89 (s, 3H), 2.59 (br s, 2H), 1.99 (br s, 2H), 1.50 (t, J=6.0 Hz, 2H), 1.18 (d, J=9.2 Hz, 6H), 1.04 (s, 6H).

HPLC (Method A): 8.50 min, 95.33%, 254.0 nm.

LCMS (Method A): 2.796 min, 99.71%, 254.0 nm, MS: ES+353.4.

### Example 147—Synthesis of N-isopropyl-6-methoxy-8-(spiro[2.5]oct-5-en-6-yl)quinoline-3-carboxamide (Compound 156)

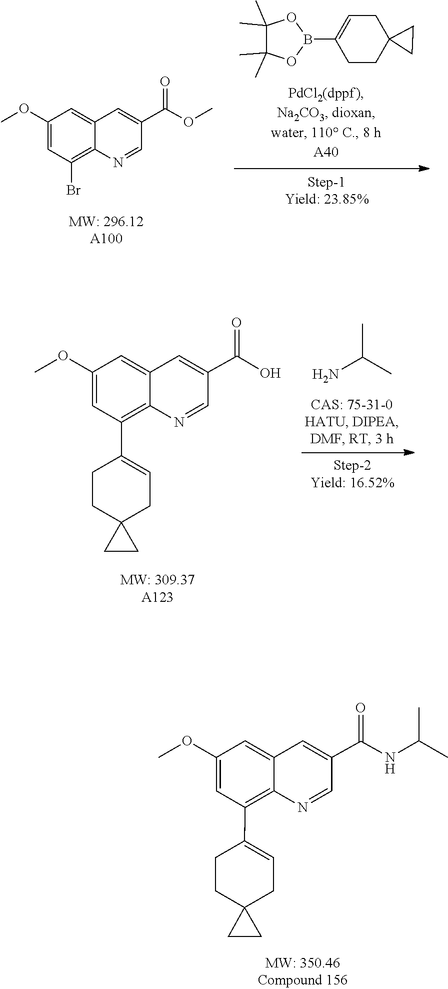

### Step-1

To a stirred solution of 4,4, 5, 5-tetramethyl-2-(spiro[2.5]oct-5-en-6-yl)-1, 3, 2-dioxaborolane (A40) (1.97 g, 8.442 mmol, 1.0 eq.), methyl 8-bromo-6-methoxyquinoline-3-carboxylate (A100) (2.50 g, 8.442 mmol, 1.0 eq.) and Na₂CO₃ (1.77 g, 16.884 mmol, 2.0 eq.) in Dioxane (40 mL) and water (10 mL) was added Pd(dppf)Cl₂·DCM (1.23 g, 1.688 mmol, 0.2 eq.) under N₂ Atmosphere. The resulting mixture was stirred at 110° C. for 8 h. TLC indicated the completion of reaction; the resulting reaction mixture was filtered through celite bed and concentrated under reduced pressure to afford crude. The obtained crude material was purified by flash column chromatography using silica gel (100-200 mesh) as stationary phase (desired product eluted in 78% EtOAc in Hexane) to afford 6-methoxy-8-(spiro[2.5]oct-5-en-6-yl)quinoline-3-carboxylic acid (A123) (0.67 g, 2.16 mmol, Yield: 25.65%).

¹H NMR (DMSO-d₆, 400 MHz): δ ppm, 13.42 (br s, 1H), 9.14 (d, J=2.4 Hz, 1H), 8.82 (d, J=2.4 Hz, 1H), 7.50 (d, J=2.8 Hz, 1H), 7.28 (d, J=2.8 Hz, 1H), 5.90 (br s, 1H), 3.91 (s, 3H), 2.66 (br s, 2H), 2.11 (br s, 2H), 1.54 (t, J=6.0 Hz, 2H), 0.39 (br s, 4H).

LCMS (Method A): 2.496 min, 97.37%, 254.0 nm, MS: ES+310.4.

### Step 2

To a stirred solution of 6-methoxy-8-(spiro[2.5]oct-5-en-6-yl)quinoline-3-carboxylic acid (A123) (0.075 g, 0.242 mmol, 1.0 eq.) in DMF (10 mL), was added isopropyl amine (0.018 g, 0.242 mmol, 1.0 eq.), DIPEA (0.095 g, 0.726 mmol, 3.0 eq.) and HATU (0.138 g, 0.363 mmol, 1.5 eq.). The resulting mixture was stirred at room temperature for 3 h. TLC indicated the completion of reaction; the resulting reaction mixture was diluted with water (50 mL) and extracted by EtOAc (2×75 mL). Organic phase was dried over Na₂SO₄ and concentrated under reduced pressure to afford crude. The obtained crude material was purified by flash column chromatography using silica gel (100-200 mesh) as stationary phase (desired product eluted in 26% EtOAc in Hexane) to afford N-isopropyl-6-methoxy-8-(spiro[2.5]oct-5-en-6-yl)quinoline-3-carboxamide (Compound 156) (0.014 g, 0.04 mmol, 16.52%)

¹H NMR (DMSO-d₆, 400 MHz): δ ppm, 9.08 (d, J=2.0 Hz, 1H), 8.64 (d, J=2.0 Hz, 1H), 8.51 (d, J=7.6 Hz, 1H), 7.36 (d, J=2.8 Hz, 1H), 7.22 (d, J=2.8 Hz, 1H), 5.90 (br s, 1H), 4.17-4.12 (m, 1H), 3.90 (s, 3H), 2.67-2.61 (m, 2H), 2.12 (br s, 2H), 1.54 (t, J=6.0 Hz, 2H), 1.21 (d, J=6.4 Hz, 6H), 0.39 (br s, 4H).

HPLC (Method A): 9.874 min, 96.18%, 254.0 nm.

LCMS (Method A): 2.618 min, 97.24%, 254.0 nm, MS: ES+351.4.

### Example 148—Synthesis of (R)-8-(4,4-dimethylcyclohex-1-en-1-yl)-N-(1-hydroxypropan-2-yl)-6-methoxyquinoline-3-carboxamide (Compound 157)

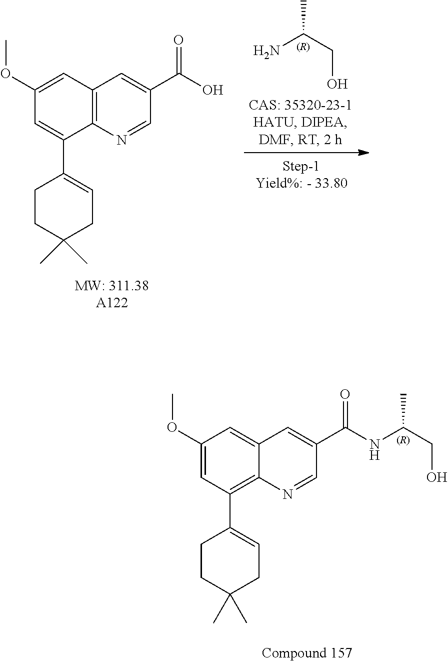

### Step-1

To a stirred solution of A122 (0.1 g, 0.321 mmol, 1.0 eq.), in DMF (2 mL), was added HATU (0.244 g, 0.643 mmol, 2.0 eq.) and DIPEA (0.124 g, 0.963 mmol, 3.0 eq.) respectively at 0° C., followed by addition of CAS No: 35320-23-1 (R)-2- Aminopropan-1-ol (0.036 g, 0.481 mmol, 1.5 eq.) and allow the reaction mixture to stir at RT for 2 h. TLC indicated the completion of reaction; the resulting reaction mixture was diluted with ice water (50 mL) and extracted with EtOAc (3×50 mL). The combined organic layers were dried over Na₂SO₄ and concentrated under reduced pressure to afford crude. The crude was purified by flash column chromatography on silica gel (100-200 mesh) (Desired product eluted in 45% Ethyl Acetate in Hexane) to afford (R)-8-(4, 4-dimethylcyclohex-1-en-1-yl)-N-(1-hydroxypropan-2-yl)-6-methoxyquinoline-3-carboxamide (Compound 157) (0.040 g, 0.108 mmol, Yield: 33.80%).

¹H NMR (DMSO-d₆, 400 MHz): δ ppm, 9.09 (d, J=1.6 Hz, 1H), 8.65 (d, J=1.6 Hz, 1H), 8.39 (d, J=7.6 Hz, 1H), 7.35 (d, J=2.4 Hz, 1H), 7.19 (d, J=2.8 Hz, 1H), 5.75 (br s, 1H), 4.77 (t, J=5.6 Hz, 1H), 4.09-4.05 (m, 1H), 3.90 (s, 3H), 3.51-3.47 (m, 1H), 2.60 (br s, 2H), 2.00 (br s, 2H), 1.50 (t, J=6.0 Hz, 1H), 1.23 (s, 2H), 1.16 (d, J=6.4 Hz, 3H), 1.04 (s, 6H).

¹H NMR (MeOD, 400 MHz): δ ppm, 9.06 (d, J=2.4 Hz, 1H), 8.62 (d, J=2.0 Hz, 1H), 7.27-7.23 (m, 2H), 5.78 (br. s, 1H), 4.31-4.24 (m, 1H), 3.96 (s, 3H), 3.67-3.65 (m, 2H), 2.59 (br. s, 1H), 2.07 (br. s, 2H), 1.62 (t, J=6.4 Hz, 1H), 1.32-1.30 (d, J=6.4 Hz, 4H), 1.09 (s, 6H). Note: —CONH and —OH proton exchanged in MeOD NMR.

LCMS (Method A): 2.40, 97.75%, 254 nm, MS: ES+369 (M), (M+1)

HPLC (Method A): 8.96, 100%, 254 nm,

### Example 149—Synthesis of N-((1H-pyrazol-4-yl)methyl)-8-(4, 4-dimethylcyclohex-1-en-1-yl)-6-methoxyquinoline-3-carboxamide (Compound 158)

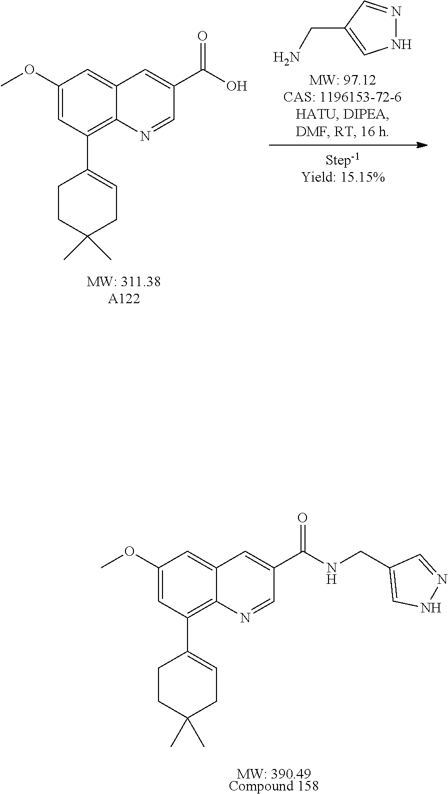 [SMILES: COc1cc(C2=CCC(C)(C)CC2)c2ncc(C(=O)O)cc2c1 ; NCc1cn[nH]c1 ; COc1cc(C2=CCC(C)(C)CC2)c2ncc(C(=O)NCc3cn[nH]c3)cc2c1 ; "MW: 390.49Compound 158"]

### Step-1

To a stirred solution of 8-(4, 4-dimethylcyclohex-1-en-1-yl)-6-methoxyquinoline-3-carboxylic acid (A122) (0.100 g, 0.321 mmol, 1.0 eq.) in DMF (10 mL), was added (1H-pyrazol-4-yl) methanamine CAS: 1196153-72-6 (0.042 g, 0.321 mmol, 1.0 eq.), DIPEA (0.125 g, 0.963 mmol, 3.0 eq.) and HATU (0.182 g, 0.481 mmol, 1.5 eq.). The resulting mixture was stirred at room temperature for 16 h. TLC indicated the completion of reaction; the resulting reaction mixture was diluted with water (50 mL) and extracted by EtOAc (2×75 mL). Organic phase was dried over Na₂SO₄ and concentrated under vacuum to afford crude. The obtained crude material was purified by flash column chromatography using silica gel (100-200 mesh) as stationary phase (desired product eluted in 26% EtOAc in Hexane) to afford N-((1H-pyrazol-4-yl)methyl)-8-(4,4-dimethylcyclohex-1-en-1-yl)-6-methoxyquinoline-3-carboxamide (Compound 158) (0.019 g, 0.048 mmol, Yield: 15.15%) 1H NMR (MeOD, 400 MHz): δ ppm, 9.09 (d, J=2.4 Hz, 1H), 8.64 (d, J=2.0 Hz, 1H), 7.64 (m, 1H), 7.27 (dd, J=9.6, 2.8 Hz, 2H), 6.37 (br s, 1H), 5.79 (br s, 1H), 4.70 (br s, 2H), 3.99 (s, 3H), 2.59 (br s, 2H), 2.07 (br s, 2H), 1.63 (t, J=6.4 Hz, 2H), 1.08 (s, 6H). Note: —CONH and —NH proton exchanged in MeOD NMR.

¹H NMR (DMSO-d₆ 400 MHz): δ ppm, 12.62 (br s, 1H), 9.18 (br s, 1H), 9.10 (d, J=1.6 Hz, 1H), 8.69 (d, J=2.0 Hz, 1H), 7.66 (br s, 1H), 7.35 (d, J=2.4 Hz, 1H), 7.20 (d, J=2.8 Hz, 1H), 6.22 (br s, 1H), 5.77 (br. s, 1H), 4.53 (br s, 2H), 3.90 (s, 3H), 2.67-2.59 (m, 2H), 1.99 (br s, 2H), 1.51 (t, J=6.4 Hz, 2H), 1.04 (s, 6H).

HPLC (Method A): 8.862 min, 95.24%, 254.0 nm.

LCMS (Method A): 2.462 min, 95.89%, 254.0 nm, MS: ES+391.5 (M+1)

### Example 150—Synthesis of (R)-8-(4, 4-dimethylcyclohex-1-en-1-yl)-6-methoxy-N-(1-(oxazol-2-yl)ethyl)quinoline-3-carboxamide (Compound 159)

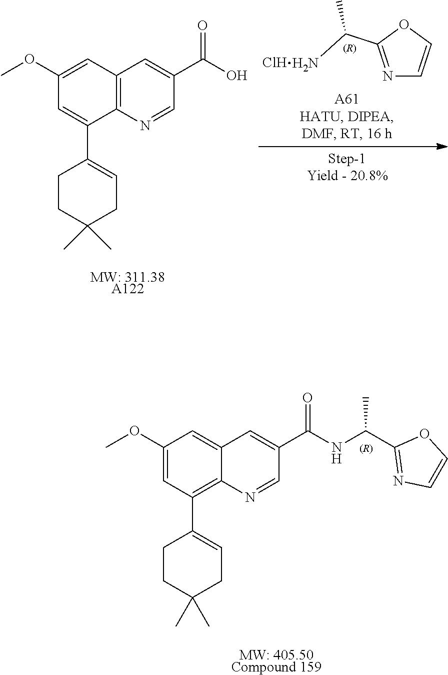

### Step-1

To a stirred solution of A122 (0.07 g, 0.22 mmol, 1.0 eq.), (A61) (0.03 g, 0.27 mmol, 1.2 eq.) in DMF (4 mL), was added HATU (0.128 g, 0.337 mmol, 1.5 eq.) and DIPEA (0.11 mL, 0.67 mmol, 3.0 eq.), under the N₂ atmosphere. Then the resulting mixture was stirred at room temperature for 16 h. TLC indicated the completion of reaction; After 16 h. water (20 mL) was added and resulting reaction mixture was extracted with ethyl acetate (3×30 mL). The combined organic layers were dried over Na₂SO₄ and concentrated under reduced pressure to afford crude. The crude was purified by manual column chromatography using silica (100-200 mesh) as stationary phase (10% MDC:MeOH) to afford (R)-8-(4, 4-dimethylcyclohex-1-en-1-yl)-6-methoxy-N-(1-(oxazol-2-yl)ethyl)quinoline-3-carboxamide (Compound 159) (0.019 g, 0.04 mmol, Yield 20.6%)

¹H NMR (MeOD, 400 MHz): δ ppm 9.08 (d, J=2.0 Hz, 2H), 8.67 (d, J=2.0 Hz, 2H), (7.92 (s, 1H), 7.26 (d, J=2.8 Hz, 2H), 7.24 (d, J=2.8 Hz, 2H) 7.18 (s, 1H), 5.79 (br. s, 1H), 5.51-5.45 (m, 1H), 3.96 (s, 3H), 2.59 (br. d, J=2.0 Hz, 2H), 2.80 (br. s, 2H), 1.72 (d, J=7.2 Hz, 3H), 1.63 (t, J=2.8 Hz, 2H), 1.09 (s, 6H).

Note: —CONH proton exchanged in MeOD NMR.

¹H NMR (DMSO, 400 MHz): δ ppm 9.28 (d, J=8.0 Hz, 1H), 9.10 (d, J=2.0 Hz, 1H), 8.70 (d, J=2.0 Hz, 1H), 8.08 (s, 1H), 7.37 (d, J=2.8 Hz, 1H), 7.20 (d, J=2.8 Hz, 1H), 7.18 (s, 1H), 5.76 (br s, 1H), 5.38-5.34 (m, 1H), 3.90 (s, 3H), 2.00 (s, 2H), 1.59 (d, J=7.2 Hz, 3H), 1.51 (t, J=6.4 Hz, 2H), 1.04 (s, 6H). Note: —CH₂ proton merged in DMSO Solvent peak.

LCMS (Method A): 2.68 min, 99.83%, 210 nm M+H=406.37

HPLC (Method A): 9.88 min, 99.19%, 254 nm

Chiral HPLC: 3.90 min, 98.73%, 248 nm

### Example 151—Synthesis of(S)-8-(4,4-dimethylcyclohex-1-en-1-yl)-N-(1-hydroxy-3-methoxypropan-2-yl)-6-methoxyquinoline-3-carboxamide (Compound 160)

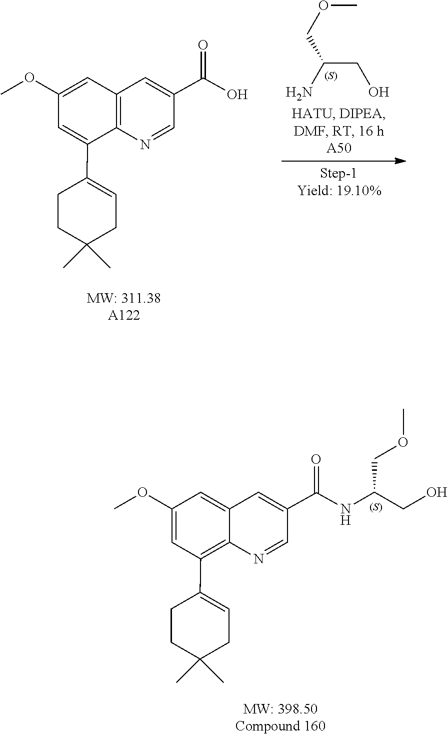

### Step-1

To a stirred solution of 8-(4, 4-dimethylcyclohex-1-en-1-yl)-6-methoxyquinoline-3-carboxylic acid (A122) (0.09 g, 0.289 mmol, 1.0 eq.) and HATU (0.16 g, 0.433 mmol, 1.5 eq.) along with DIPEA (0.15 mL, 0.867 mmol, 3.0 eq.) in DMF (1 mL) at 0° C. under nitrogen atmosphere was added (A50) (S)-2-amino-3-methoxypropan-1-ol (0.060 g, 0.578 mmol, 2.0 eq.) and resulting mixture was stirred for 16 h at room temperature. TLC indicated the completion of reaction; the resulting reaction mixture was diluted with ice-cold water (10 mL) and extracted with EA (3×20 mL). The combined organic layers were dried over Na₂SO₄ and concentrated under reduced pressure to afford crude. The obtained crude material was purified by flash column chromatography using silica (100-200 mesh) as stationary phase (desired product eluted in 80% EA in Hex) to afford(S)-8-(4,4-dimethylcyclohex-1-en-1-yl)-N-(1-hydroxy-3-methoxypropan-2-yl)-6-methoxyquinoline-3-carboxamide (Compound 160) (0.022 g, 0.055 mmol, Yield: 19%).

¹H NMR (MeOD, 400 MHz): δ ppm, 9.06 (d, J=2.4 Hz, 1H), 8.63 (d, J=2.0 Hz, 1H), 8.44 (d, J=8.0 Hz, 1H), 7.23 (d, J=2.8 Hz, 1H), 5.76 (br s, 1H), 4.39-4.36 (m, 1H), 3.90 (s, 3H), 3.54-3.34 (m, 4H), 3.28 (s, 3H), 2.59-2.58 (m, 2H). 2.07 (s, 2H), 1.62 (t, J=12.4 Hz, 3H), 1.09 (s, 6H). Note: —CONH and —OH proton exchanged in MeOD NMR.

¹H NMR (DMSO-d₆, 400 MHz): δ ppm, 9.10 (d, J=2.0 Hz, 1H), 8.67 (d, J=1.6 Hz, 1H), 8.44 (d, J=8.0 Hz, 1H), 7.36 (d, J=2.8 Hz, 1H), 7.20 (d, J=2.8 Hz, 2H), 5.76 (br s, 1H), 4.81 (t, J=11.2 Hz, 1H), 4.21-4.16 (m, 1H), 3.90 (s, 3H), 3.54-3.46 (m, 4H), 3.28 (s, 3H), 2.67-2.57 (m, 1H), 2.00 (br s, 2H), 1.50 (t, J=12.8 Hz, 2H), 1.10 (s, 6H).

LCMS (Method A): 2.417 min, 100%, 254.0 nm, MS: ES+399 (M+1)

HPLC (Method A): 8.90 min, 100%, 254.0 nm

CHIRAL HPLC (Method A): 4.29 min, 99.17%, 248.0 nm

### Example 152—Synthesis of 8-(4,4-dimethylcyclohex-1-en-1-yl)-N-(3-(methyl sulfonamido)propyl)quinoline-3-carboxamide (Compound 161)

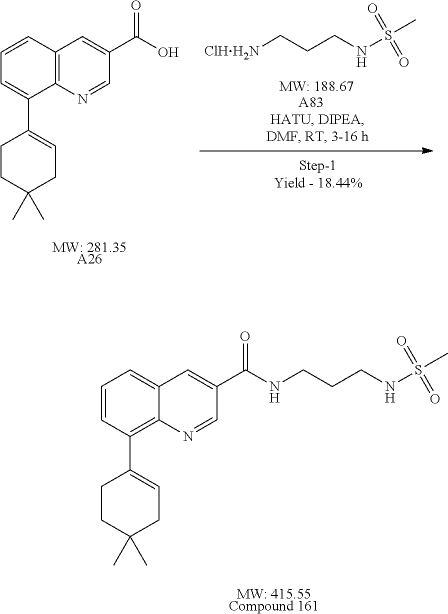

### Step-1

To a stirred solution of A26 (0.07 g, 0.24 mmol, 1.0 eq.), A83 N-(3-aminopropyl) methanesulfonamide hydrochloride (0.056 g, 0.29 mmol, 1.2 eq.), in DMF (2 mL), HATU (0.12 g, 0.36 mmol, 1.5 eq.), DIPEA (0.16 mL, 0.72 mmol, 3.0 eq.), was added to the reaction mixture under the N₂ atmosphere. The resulting mixture was stirred at room temperature for 16 h. TLC indicated the completion of reaction; the resulting reaction mixture was extracted with ethyl acetate (2×30 mL). The combined organic layers dried over Na₂SO₄ and concentrated under reduced pressure to afford crude. The crude was purified by manual column chromatography using silica (60-120 Mesh) as stationary phase (30% EtOAc:Hexane) to afford 8-(4,4-dimethylcyclohex-1-en-1-yl)-N-(3-(methylsulfonamido)propyl)quinoline-3-carboxamide (Compound 161) (0.019 g, 0.045 mmol, Yield 18.44%)

¹H NMR (MeOD, 400 MHz): δ ppm 9.24 (d, J=2.0 Hz, 1H), 8.73 (d, J=2.4 Hz, 1H), 7.93 (dd, J=6.8, 6.8 Hz, 1H), 7.64-7.62 (m, 2H), 5.78 (br s, 1H), 3.57 (t, J=13.6 Hz, 2H), 3.21 (t, J=13.6 Hz, 2H), 2.61 (d, J=1.6 Hz, 2H), 2.09 (s, 2H), 1.94-1.91 (m, 2H), 1.64 (t, J=12.4 Hz, 2H), 1.10 (s, 6H). Note: —CONH proton exchanged in MeOD NMR.

¹H NMR (DMSO, 400 MHz): δ ppm 9.25 (d, J=2.4 Hz, 1H), 8.80-8.77 (m, 2H), 7.99-7.96 (m, 1H), 7.61 (t, J=5.2 Hz, 2H), 7.03 (t, J=12.0 Hz, 1H), 5.74 (br s, 1H), 3.41-3.34 (m, 2H), 3.06-3.01 (m, 2H), 2.91 (s, 3H), 2.67-2.60 (m, 2H), 2.01 (br s, 2H), 1.79-1.74 (m, 2H), 1.52-1.49 (m, 2H), 1.05 (s, 6H).

LCMS (Method A)-2.504 min, 98.69%, 254.0 nm

HPLC (Method A)-8.96 min, 99.49%, 210 nm

### Example 153—Synthesis of 8-(2,2-difluorospiro[3.5]non-6-en-7-yl)-N-isopropyl quinoline-3-carboxamide (Compound 162)

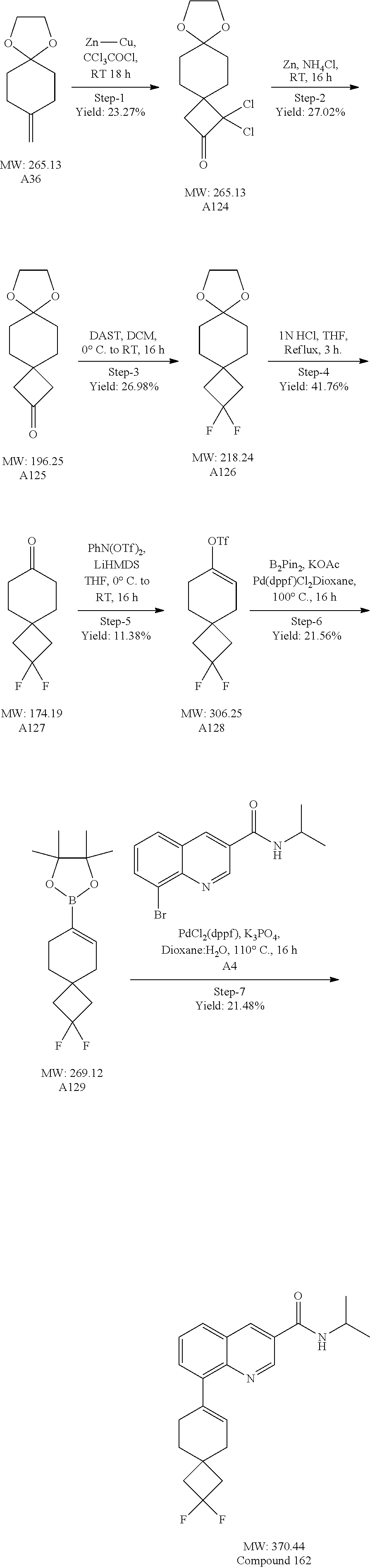

### Step 1

To a stirred solution of A36 (0.3 g, 1.945 mmol, 1.0 eq.) & Zn—Cu couple (1.254 g, 9.727 mmol, 5.0 eq.), in DME (5.0 mL) was added Cl₃CCOCl (0.56 mL g, 5.058 mmol, 2.6 eq.) dropwise to the reaction mixture under the N₂ atmosphere at RT. Then the resulting mixture was continue stirred at RT for 18 h. TLC indicated the completion of reaction; the resulting reaction mixture was quenched using aq. NH₄Cl then extracted with EtOAc (30 mL×4). The combined organic layers were washed with brine (40 mL) dried over Na₂SO₄ and concentrated under reduced pressure to afford crude. The crude was purified by manual column chromatography using silica (230-400 mesh) as stationary phase (15% Ethyl acetate in hexane) to afford 1, 1-dichloro-8, 11-dioxadispiro[3.2.4⁷.2⁴]tridecan-2-one (A124) (0.12 g, 0.453 mmol, Yield: 23.27%)

¹H NMR (CDCl₃, 400 MHz): δ ppm 3.99-3.96 (m, 4H), 2.88-2.81 (m, 2H), 1.97-1.69 (m, 8H).

### Step 2

To a stirred solution of 1, 1-dichloro-8, 11-dioxadispiro[3.2.4⁷.2⁴]tridecan-2-one (A124) (0.1 g, 0.377 mmol, 1.0 eq.), in Methanol (1.0 mL) and Saturated aq. NH₄Cl (1.0 mL) was added Zn Dust (0.247 g, 3.773 mmol, 10.0 eq.) at RT and resulting reaction mixture was continue stirred for 16 h. TLC indicated the completion of reaction; After 16 h. reaction mixture was filtered through celite bed washed with EtOAc (20 mL). Combined ethyl acetate layer washed with water (20 mL) dried over Na₂SO₄ and concentrated under reduced pressure to afford crude. The obtained crude material was purified by flash column chromatography using silica (230-400 mesh) as stationary phase (desired product eluted in 15% EtOAc in Hexane) to afford 8, 11-dioxadispiro[3.2.4⁷.2⁴]tridecan-2-one (A125) (0.04 g, 0.204 mmol, Yield 54.02%).

¹H NMR (CDCl₃, 400 MHz): δ ppm, 3.87 (s, 4H), 2.78 (s, 4H), 1.72-1.69 (m, 4H), 1.57-1.54 (m, 4H).

### Step 3

To a stirred solution of 8, 11-dioxadispiro[3.2.4⁷.2⁴]tridecan-2-one (A125) (0.2 g, 1.019 mmol, 1.0 eq.) in DCM (5 Ml) was added DAST (0.34 MI, 2.548 mmol, 2.5 eq.) at 0° C. Then the resulting mixture was stirred at RT for 16 h. TLC indicated the completion of reaction; the resulting reaction mixture was quenched using ice cold water (20 Ml) and extracted with ethyl acetate (30 Ml×4). The combined organic layers were dried over Na₂SO₄ and concentrated under reduced pressure to afford crude. The obtained crude material was purified by flash column chromatography using silica gel (230-400 mesh) as stationary phase (desired product eluted in 18% EtOAc in Hexane) to afford 2, 2-difluoro-8, 11-dioxadispiro[3.2.4⁷.2⁴]tridecane (A126) (0.06 g, 0.275 mmol, Yield: 26.98%).

¹H NMR (CDCl₃, 400 MHz): δ ppm 3.96 (m, 4H), 2.36 (t, J=5.6 Hz, 4H), 1.76-1.73 (m, 4H), 1.63-1.59 (m, 4H).

### Step 4

To a stirred solution of 2, 2-difluoro-8, 11-dioxadispiro[3.2.4⁷.2⁴]tridecane (A126) (0.06 g, 0.275 mmol, 1.0 eq.) in THF (1.0 mL) was added IN HCl solution (1.0 mL) to the reaction mixture at room temperature. Then reaction mixture stirred at 70° C. for 3 h. TLC indicated the completion of reaction; the resulting reaction mixture was diluted with water (50 mL) and extracted with ethyl acetate (3×10 mL). The combined organic layers were dried over Na₂SO₄ and concentrated under reduced pressure to afford crude. The obtained crude material was purified by flash column chromatography using silica (230-400 Mesh) as stationary phase (desired product eluted in 10% EtOAc in Hexane) to afford 2,2-difluorospiro[3.5]nonan-7-one (A127) (0.020 g, 0.114 mmol, Yield: 41.76%)

¹H NMR (CDCl₃, 400 MHz): δ ppm 2.52 (t, J=6 Hz, 4H), 2.36 (t, J=6.8 Hz, 4H), 2.00 (t, J=6.8 Hz, 4H).

### Step 5

To a stirred solution of 2, 2-difluorospiro[3.5]nonan-7-one (A127) (0.025 g, 0.144 mmol, 1.0 eq.) in THF (3 mL) was added LiHMDS (0.158 mL, 0.158 mmol, 1.1 eq.) under the N₂ atmosphere at 0° C. and stirred for 1 h. Followed by addition of CAS: 37595-74-7 (0.056 g, 0.158 mmol, 1.1 eq.), was added to the reaction mixture. Then reaction mixture stirred at RT for 16 h. TLC indicated the completion of reaction; the resulting reaction mixture was quenched using H₂O extracted with ethyl acetate (4×5 mL). The combined organic layers were dried over Na₂SO₄ and concentrated under reduced pressure to afford crude. The crude was purified by manual column chromatography using silica (230-400 mesh) as stationary phase (desired product eluted in 20% Ethyl acetate in hexane) to afford 2, 2-difluorospiro[3.5]non-6-en-7-yl trifluoromethanesulfonate (A128) (0.005 g, 0.0163 mmol, Yield: 11.38%).

¹H NMR (CDCl₃, 400 MHz): δ ppm 5.74-5.72 (m, 1H), 2.46-2.35 (m, 8H), 1.90 (t, J=6.4 Hz, 2H).

### Step 6

To a stirred solution of 2, 2-difluorospiro[3.5]non-6-en-7-yl trifluoromethanesulfonate (A128) (0.1 g, 0.327 mmol, 1.0 eq.) in Dioxane (2 mL) was added B₂Pin₂ (0.91 g, 0.359 mmol, 1.1 eq.) and KOAc (0.096 g, 0.980 mmol, 3.0 eq.) under N₂ atmosphere at room temperature. Then the reaction mixture was purged with N₂ gas for 5 min. followed by addition of PdCl₂ (dppf). DCM (0.014 g, 0.0195 mmol, 0.06 eq.) and the reaction was allowed to run overnight at 100° C. TLC indicated the completion of reaction; the resulting reaction mixture was diluted with water (10 mL) and extracted with ethyl acetate (3×10 mL). The combined organic layers were dried over Na₂SO₄ and concentrated under reduced pressure to afford crude. The crude was purified by flash column chromatography using silica gel (230-400 mesh) as stationary phase (desired product eluted in 5% Ethyl acetate in hexane) to afford 2-(2, 2-difluorospiro[3.5]non-6-en-7-yl)-4,4, 5, 5-tetramethyl-1, 3, 2-dioxaborolane (A129) (0.02 g, 0.074 mmol, Yield: 21.56%)

¹H NMR (CDCl₃, 400 MHz): δ ppm 6.48 (s, 1H), 2.37-2.28 (m, 4H), 2.25-2.24 (m, 2H), 2.22-2.18 (m, 2H), 1.67 (t, J=6.0 Hz, 2H). 1.25 (s, 12H).

### Step 7

To a stirred solution of 2-(2, 2-difluorospiro[3.5]non-6-en-7-yl)-4,4, 5, 5-tetramethyl-1, 3, 2-dioxaborolane (A129) (0.302 g, 1.126 mmol, 1.1 eq.), A4 (0.3 g, 1.023 mmol, 1.0 eq.) in Dioxane (2 Ml) & H₂O (0.5 Ml), was added K₃PO₄ (0.652 g, 3.069 mmol, 3.0 eq.) and purged reaction mixture with N₂ gas for 5 min. Then PdCl₂ (dppf). DCM (0.037 g, 0.051 mmol, 0.05 eq.) was added to the reaction mixture under the N₂ atmosphere at room temperature and the reaction was allowed to run overnight at 110° C. TLC indicated the completion of reaction; the resulting reaction mixture was diluted with water (20 Ml) & extracted with ethyl acetate (3×20 Ml). The combined organic layers were dried over Na₂SO₄ and concentrated under reduced pressure to afford crude. The crude was purified by flash column chromatography using silica (230-400 mesh) as stationary phase (desired product eluted in 30% Ethyl acetate in hexane) to afford 8-(2, 2-difluorospiro[3.5]non-6-en-7-yl)-N-isopropylquinoline-3-carboxamide (Compound 162) (0.084 g, 0.227 mmol, Yield: 21.48%).

¹H NMR (DMSO-d₆, 400 MHz): δ ppm 9.25 (s, 1H), 8.77 (s, 1H), 8.57 (d, J=7.6 Hz, 1H), 7.98 (t, J=4.8 Hz, 1H), 7.61 (d, J=4.8 Hz, 2H), 5.79 (br s, 1H), 4.19-4.14 (m, 1H), 2.69 (br s, 2H), 2.46-2.38 (m, 6H), 1.85 (t, J=6.0 Hz, 2H), 1.22 (d, J=6.8 Hz, 6H).

¹H NMR (MeOD, 400 MHz): δ ppm 9.23 (d, J=2.4 Hz, 1H), 8.72 (d, J=2.0 Hz, 1H), 7.94 (dd, J=2.8, 6.4 Hz, 1H), 7.65-7.62 (m, 2H), 5.81 (br s, 1H), 4.33-4.26 (m, 1H), 2.69-2.68 (m, 2H), 2.53-2.45 (m, 6H), 1.97 (t, J=6.0 Hz, 2H), 1.33 (d, J=6.4 Hz, 6H).

LCMS (Method A): 2.637 min, 99.25%, 254.0 nm, MS: ES+371.2 (M+1).

HPLC (Method-A): 9.45 min, 99.04%, 254.0 nm

### Example 154—Synthesis of(S)-8-(2,2-difluorospiro[3.5]non-6-en-7-yl)-N-(1-(pyridin-2-yl)ethyl)quinoline-3-carboxamide (Compound 163)

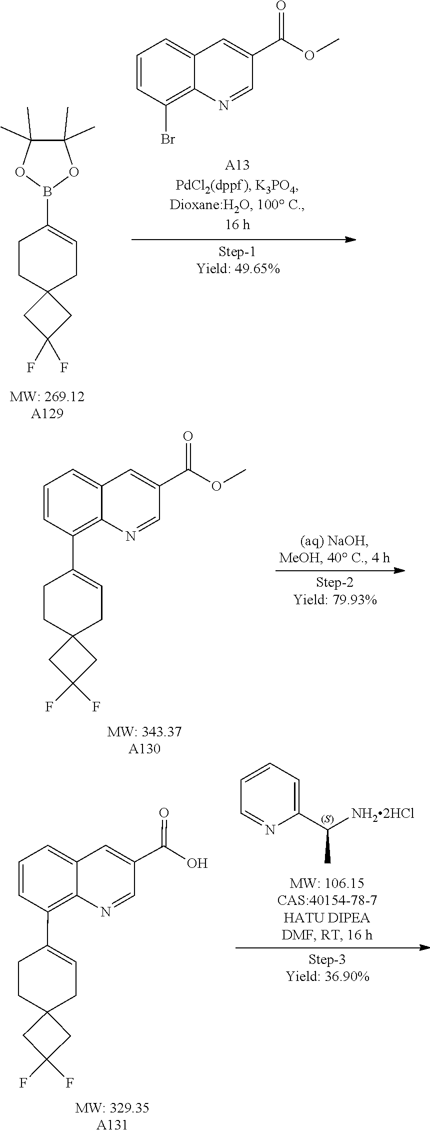

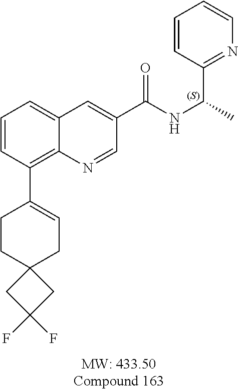

### Step-1

To a stirred solution of methyl 8-bromoquinoline-3-carboxylate (A13) (0.45 g, 1.691 mmol, 1.0 eq.), A129 (0.5 g, 1.860 mmol, 1.1 eq.) in Dioxane (4 mL) and H₂O (2 mL) was added K₃PO₄ (1.074 g, 5.062 mmol, 3.0 eq.) and then reaction mixture purged with N₂ gas for 5 min, followed by addition of PdCl₂ (dppf).DCM complex (0.619 g, 0.844 mmol, 0.5 eq.) and resulting mixture was stirred at 110° C. for 16 h. TLC indicated the completion of reaction; the resulting reaction mixture was diluted with water (30 mL) and extracted with ethyl acetate (3×30 mL). The combined organic layers were dried over Na₂SO₄ and concentrated under reduced pressure to afford crude. The crude was purified by flash column chromatography using silica gel (230-400 mesh) as stationary phase (30% Ethyl acetate in hexane) to afford methyl 8-(2, 2-difluorospiro[3.5]non-6-en-7-yl)quinoline-3-carboxylate (A130) (0.3 g, 0.874 mmol, Yield: 49.65%)

¹H NMR (MeOD, 400 MHz): δ ppm 9.35 (d, J=2.0 Hz, 1H), 8.95 (d, J=2.4 Hz, 1H), 7.99 (dd, J=1.6, 8.0 Hz, 1H), 7.70-7.62 (m, 2H), 5.80 (br s, 1H), 4.03 (s, 3H), 2.70-2.67 (m, 2H), 2.53-2.45 (m, 6H), 1.97 (t, J=6.0 Hz, 2H).

LCMS (Method A): 2.900 min, 91.28%, 254.0 nm, MS: ES+344.2 (M+1).

### Step-2

To a stirred solution of methyl 8-(2, 2-difluorospiro[3.5]non-6-en-7-yl)quinoline-3-carboxylate (A130) (0.3 g, 0.874 mmol, 1.0 eq.) in MeOH (2.4 mL) and H₂O (0.3 mL) was added NaOH (0.175 g, 4.368 mmol, 5.0 eq.). Then the resulting mixture was stirred at 40° C. for 4 h. TLC indicated the completion of reaction; the resulting reaction mixture was concentrated under reduced pressure then obtained residue was acidified by 1N HCl (10 mL) and extracted with EtOAc (4×30 mL). The combined organic layers were dried over Na₂SO₄ and concentrated under reduced pressure to afford 8-(2, 2-difluorospiro[3.5]non-6-en-7-yl)quinoline-3-carboxylic acid (A131) (0.23 g, 0.699 mmol, Yield 79.93%)

¹H NMR (DMSO-d₆, 400 MHz): δ ppm 13.51 (br s, 1H), 9.29 (d, J=2.0 Hz, 1H), 8.95 (d, J=2.0 Hz, 1H), 8.10-8.08 (m, 1H), 7.67-7.62 (m, 2H), 5.79 (br s, 1H), 2.68 (s, 2H), 2.51-2.38 (m, 4H), 1.84 (t, J=6.0 Hz, 2H).

¹H NMR (MeOD, 400 MHz): δ ppm 9.36 (d, J=2.0 Hz, 1H), 8.96 (d, J=1.6 Hz, 1H), 7.99 (d, J=6.8 Hz, 1H), 7.69-7.62 (m, 2H), 5.81 (br s, 1H), 2.69-2.68 (m, 2H), 2.53-2.45 (m, 6H), 1.97 (t, J=6.4 Hz, 2H

LCMS (Method A): 2.476 min, 91.40%, 254.0 nm, MS: ES+330.3 (M+1).

### Step-3

To a solution of 8-(2, 2-difluorospiro[3.5]non-6-en-7-yl)quinoline-3-carboxylic acid (A131) (0.07 g, 0.213 mmol, 1.0 eq.), HATU (0.242 g, 0.638 mmol, 3.0 eq.) and DIPEA (0.22 mL, 1.275 mmol, 6.0 eq.) in DMF (2 mL) at 0° C. under nitrogen atmosphere stirred for 5 min, was added CAS: 40154-78-7 (0.049 g, 0.468 mmol, 2.2 eq.) under Nitrogen. The resulting mixture was stirred for 16 h at room temperature. TLC indicated the completion of reaction; the resulting reaction mixture was quenched with ice-cold water (10 mL) and extracted with EtOAc (3×10 mL). The combined organic layers were dried over Na₂SO₄ and concentrated under reduced pressure to afford crude. The obtained crude material was purified by flash column chromatography using silica gel (230-400 mesh) as stationary phase (desired product eluted in 80% Ethyl acetate in hexane) to afford(S)-8-(2, 2-difluorospiro[3.5]non-6-en-7-yl)-N-(1-(pyridin-2-yl)ethyl)quinoline-3-carboxamide (Compound 163) (0.034 g, 0.078 mmol, Yield: 36.90%).

¹H NMR (DMSO-d₆, 400 MHz): δ ppm 9.31 (d, J=2.0 Hz, 1H), 9.19 (d, J=7.6 Hz, 1H), 8.87 (d, J=2.0 Hz, 1H), 8.54 (d, J=4.4 Hz, 1H), 8.00 (t, J=4.8 Hz, 1H), 7.78 (t, J=6.0 Hz, 1H), 7.63 (d, J=4.8 Hz, 2H), 7.48 (d, J=7.6 Hz, 1H), 7.29-7.26 (m, 1H), 5.79 (br s, 1H), 5.30-5.22 (m, 1H), 2.70-2.68 (m, 4H), 2.43-2.33 (m, 4H), 1.86 (t, J=6.0 Hz, 2H), 1.56 (d, J=7.2 Hz, 3H).

LCMS (Method A): 2.284 min, 95.81%, 254.0 nm, MS: ES+434.4 (M+1)

HPLC (Method A): 9.19 min, 98.56%, 254.0 nm

### Example 155—Synthesis of 8-(2, 2-difluorospiro[3.5]non-6-en-7-yl)-N-(oxazol-2-ylmethyl)quinoline-3-carboxamide (Compound 164)

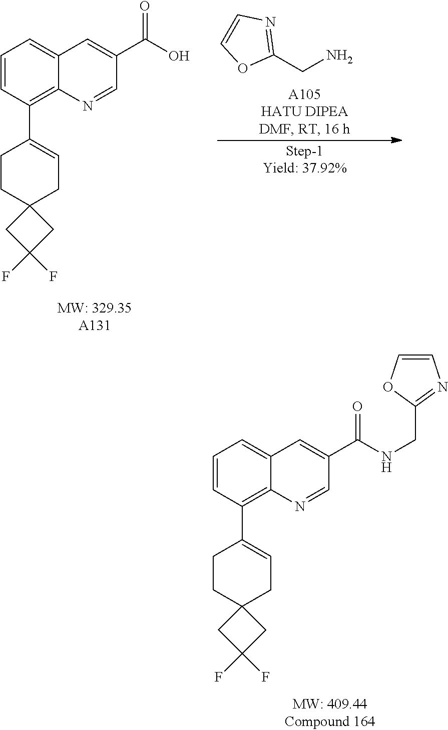 [SMILES: O=C(O)c1cnc2c(C3=CCC4(CC3)CC(F)(F)C4)cccc2c1 ; NCc1ncco1 ; O=C(NCc1ncco1)c1cnc2c(C3=CCC4(CC3)CC(F)(F)C4)cccc2c1]

### Step-1

To a solution of 8-(2, 2-difluorospiro[3.5]non-6-en-7-yl)quinoline-3-carboxylic acid (A131) (0.07 g, 0.213 mmol, 1.0 eq.), HATU (0.242 g, 0.638 mmol, 3.0 eq.) and DIPEA (0.22 mL, 1.275 mmol, 6.0 eq.) in DMF (2 mL) at 0° C. under nitrogen atmosphere stirred for 5 min, was added (A105) oxazol-2-ylmethanamine (0.063 g, 0.468 mmol, 2.2 eq.) and stirred for another 16 h at room temperature. TLC indicated the completion of reaction; the resulting reaction mixture was quenched with ice-cold water (10 mL) and extracted with EtOAc (3×10 mL). The combined organic layers were dried over Na₂SO₄ and concentrated under reduced pressure to afford crude. The obtained crude material was purified by flash column chromatography using silica gel (230-400 mesh) as stationary phase (80% Ethyl acetate in hexane as gradient) to afford 8-(2, 2-difluorospiro[3.5]non-6-en-7-yl)-N-(oxazol-2-ylmethyl)quinoline-3-carboxamide (Compound 164) (0.033 g, 0.078 mmol, Yield: 37.92%).

¹H NMR (DMSO-d₆, 400 MHz): δ ppm 9.52 (t, J=6.0 Hz, 1H), 9.28 (d, J=2.4 Hz, 1H), 8.85 (d, J=2.4 Hz, 1H), 8.09 (d, J=0.4 Hz, 1H), 8.01-7.99 (m, 1H), 7.65-7.62 (m, 2H), 7.19 (d, J=0.8 Hz, 1H), 5.79 (br s, 1H), 4.67 (d, J=6.0 Hz, 2H), 2.68-2.67 (m, 4H), 2.43-2.33 (m, 4H), 1.85 (t, J=6.0 Hz, 2H).

¹H NMR (MeOD, 400 MHz): δ ppm 9.29 (d, J=2.4 Hz, 1H), 8.81 (d, J=2.4 Hz, 1H), 7.97-7.93 (m, 2H), 7.67-7.62 (m, 2H), 7.18 (d, J=0.4 Hz 1H), 5.82-5.80 (m, 1H), 4.80 (s, 2H), 2.71-2.67 (m, 2H), 2.53-2.45 (m, 6H), 1.97 (t, J=6.0 Hz, 2H).

LCMS (Method A): 2.385 min, 99.10%, 254.0 nm, MS: ES+410.4 (M+1)

HPLC (Method A): 8.39 min, 97.95%, 254.0 nm

### Example 156—Synthesis of (R)-8-(2,2-difluorospiro[3.5]non-6-en-7-yl)-N-(1-hydroxypropan-2-yl)quinoline-3-carboxamide (Compound 165)

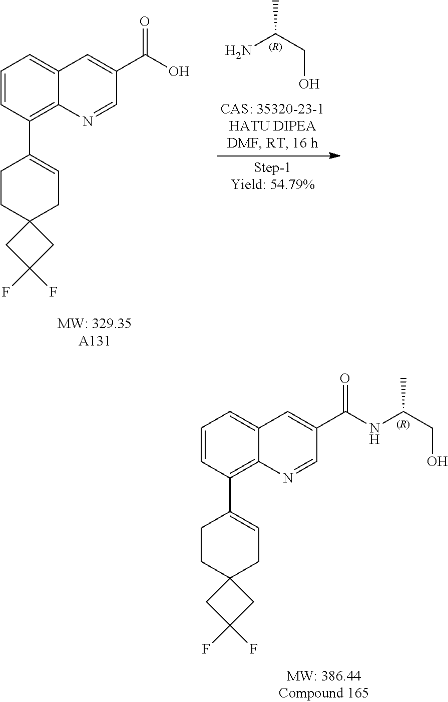

### Step-1

To a solution of 8-(2, 2-difluorospiro[3.5]non-6-en-7-yl)quinoline-3-carboxylic acid (A131) (0.07 g, 0.213 mmol, 1.0 eq.), HATU (0.242 g, 0.638 mmol, 3.0 eq.) and DIPEA (0.22 mL, 1.275 mmol, 6.0 eq.) in DMF (2 mL) at 0° C. under nitrogen atmosphere stirred for 5 min, was added CAS: 35320-23-1 (R)-2-Aminopropan-1-ol (0.035 g, 0.468 mmol, 2.2 eq.) and resulting mixture was stirred for 16 h at room temperature. TLC indicated the completion of reaction; after completion water (10 mL) was added and extracted with EtOAc (3×10 mL). The combined organic layers were dried over Na₂SO₄ and concentrated under reduced pressure to afford crude. The obtained crude material was purified by flash column chromatography using silica gel (230-400 mesh) as stationary phase (desired product eluted in 70% Ethyl acetate in hexane) to afford (R)-8-(2,2-difluorospiro[3.5]non-6-en-7-yl)-N-(1-hydroxypropan-2-yl)quinoline-3-carboxamide (Compound 165) (0.045 g, 0.116 mmol, Yield: 54.79%).

¹H NMR (DMSO-d₆, 400 MHz): δ ppm 9.26 (br s, 1H), 8.79 (br s, 1H), 8.46 (d, J=6.8 Hz, 1H), 7.98 (br s, 1H), 7.62 (br s, 2H), 5.79 (br s, 1H), 4.79 (br s, 1H), 4.08 (br s, 1H), 3.51-3.49 (m, 1H), 2.69 (br s, 3H), 2.43-2.38 (m, 3H), 1.85 (br s, 2H), 1.23-1.16 (m, 4H)

¹H NMR (MeOD, 400 MHz): δ ppm 9.26 (d, J=2.0 Hz, 1H), 8.76 (d, J=2.0 Hz, 1H), 7.94 (dd, J=2.8, 6.8 Hz, 1H), 7.64-7.62 (m, 2H), 5.80 (br s, 1H), 4.30-4.27 (m, 1H), 3.67 (d, J=4.8 Hz, 2H), 2.69-2.67 (m, 2H), 2.53-2.45 (m, 6H), 1.97 (t, J=6.0 Hz, 2H), 1.31 (d, J=6.8 Hz, 4H);

LCMS (Method A): 2.214 min, 95.77%, 254.0 nm, MS: ES+387.4 (M+1)

HPLC (Method A): 6.99 min, 96%, 254.0 nm

### Example 157—Synthesis of (R)—N-(1-(oxazol-2-yl)ethyl)-8-(spiro[2.5]oct-5-en-6-yl)quinoline-3-carboxamide (Compound 166)

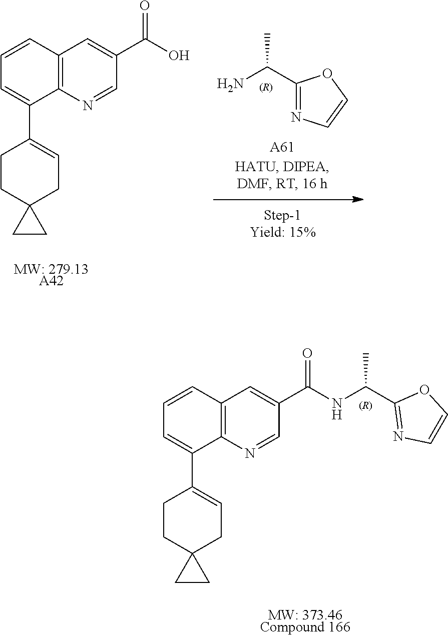

### Step-1

To a stirred solution of 8-(spiro[2.5]oct-5-en-6-yl)quinoline-3-carboxylic acid (A42) (0.090 g, 0.32 mmol, 1.0 eq.) in DMF, were added DIPEA (0.120 g, 0.96 mmol, 3.0 eq.) and HATU (0.180 g, 0.48 mmol, 1.5 eq.) at 0° C. The reaction mixture was stirred under the N₂ atmosphere. After 15 min of stirring at 0° C., (R)-1-(oxazol-2-yl)ethan-1-amine (A61) (0.050 g, 0.38 mmol, 1.2 eq.), was added at same temperature and the resulting reaction mixture was stirred at 0° C. to room temperature for 16 h. TLC indicated the completion of reaction; the resulting reaction mixture was quenched with ice-cold water (10 mL) and extracted with ethyl acetate (30 mL×2). The combined organic layers were dried over Na₂SO₄, filtered and concentrated under reduced pressure to afford crude. The crude was purified by column chromatography using silica gel (60-120 Mesh) as stationary phase (100% Ethyl acetate) to afford (R)—N-(1-(oxazol-2-yl)ethyl)-8-(spiro[2.5]oct-5-en-6-yl)quinoline-3-carboxamide (Compound 166) (0.018 g, 0.048 mmol, Yield 15%)

¹H NMR (DMSO, 400 MHz): δ ppm 9.34 (d, J=8.0 Hz, 1H), 9.28 (d, J=2.4 Hz, 1H), 8.84 (d, J=2.0 Hz, 1H), 8.08 (s, 1H), 7.99-7.97 (m, 1H), 7.67-7.61 (m, 2H), 7.19 (s, 1H), 5.89 (s, 1H), 5.38 (t, J=7.2 Hz, 1H), 2.68 (br s, 2H), 2.13 (br s, 2H), 1.61 (d, J=7.2 Hz, 3H), 1.55 (t, J=6.0 Hz, 2H), 0.40 (s, 4H).

LCMS (Method A): 2.52 min, 99.45%, 244.0 nm, MS: ES+374.3 (M+1)

HPLC (Method B): 9.17 min, 99.52%, 210 nm

Chiral HPLC-4.37 min, 97.70%, 242 nm

### Example 158—Synthesis of (R)—N-(1-hydroxypropan-2-yl)-8-(spiro[2.5]oct-5-en-6-yl)quinoline-3-carboxamide (Compound 167)

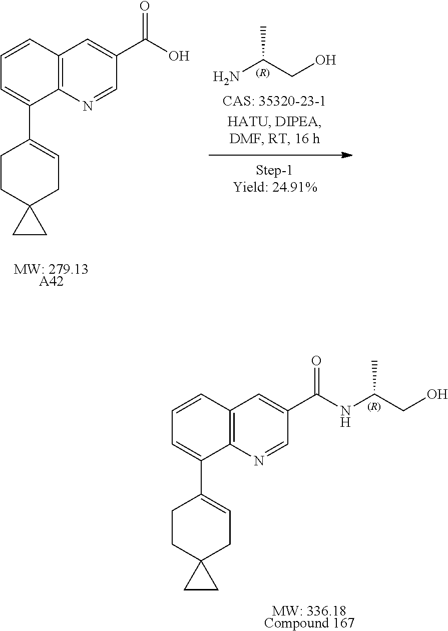

### Step-1

To a stirred solution of 8-(spiro[2.5]oct-5-en-6-yl)quinoline-3-carboxylic acid (A42) (0.090 g, 0.32 mmol, 1.0 eq.) in DMF (4.0 mL), were added DIPEA (0.120 g, 0.96 mmol, 3.0 eq.) and HATU (0.180 g, 0.48 mmol, 1.5 eq.) at 0° C. The reaction mixture was stirred under the N₂ atmosphere. After 15 min of stirring at 0° C., 2-aminoethan-1-ol (CAS No: 35320-23-1) (0.020 g, 0.38 mmol, 1.2 eq.), was added at same temperature and the resulting reaction mixture was stirred at 0° C. to room temperature for 16 h. TLC indicated the completion of reaction; the resulting reaction mixture was quenched with ice-cold water (10.0 mL) and extracted with ethyl acetate (10 mL×2). The combined organic layers were dried over Na₂SO₄, filtered and concentrated under reduced pressure to afford crude. The crude was purified by column chromatography using silica gel (60-120 Mesh) as stationary phase (100% Ethyl acetate) to afford (R)—N-(1-hydroxypropan-2-yl)-8-(spiro[2.5]oct-5-en-6-yl)quinoline-3-carboxamide (Compound 167) (0.027 g, 0.080 mmol, Yield 24.91%)

¹H NMR (MeOD, 400 MHz): δ ppm 9.25 (d, J=2.4 Hz, 1H), 8.76 (d, J=2.0 Hz, 1H), 7.93 (d, J=6.4 Hz, 1H), 7.68-7.63 (m, 2H), 5.89 (br s, 1H), 4.29-4.27 (m, 1H), 3.67 (dd, J=2.0, 6.0 Hz, 2H), 2.67 (br s, 2H), 2.19 (br s, 2H), 1.67 (t, J=6.0 Hz, 2H), 1.31 (d, J=6.8 Hz, 3H), 0.44 (s, 4H).

¹H NMR (DMSO, 400 MHz): δ ppm 9.27 (d, J=2.0 Hz, 1H), 8.79 (d, J=2.0 Hz, 1H), 8.45 (d, J=7.6 Hz, 1H), 7.97 (dd, J=2.0, 7.6 Hz, 1H), 7.64-7.59 (m, 2H), 5.88 (s, 1H), 4.79 (t, J=6.0 Hz, 1H), 4.10-4.06 (m, 1H), 3.52-3.48 (m, 1H), 3.41-3.38 (m, 1H), 2.68 (br s, 2H), 2.13 (br s, 2H), 1.55 (t, J=6.0 Hz, 2H), 1.18 (d, J=6.8 Hz, 3H) 0.40 (s, 4H).

LCMS (Method A): 2.25 min, 98.19%, 239.0 nm. MS: ES+337.4 (M+1)

HPLC (Method B): 8.14 min, 99.09%, 210.0 nm.

Chiral HPLC: 5.53 min, 97.09%, 242.0 nm

### Example 159—Synthesis of (R)—N-(4-(methylamino)-4-oxobutan-2-yl)-8-(spiro[2.5]oct-5-en-6-yl)quinoline-3-carboxamide (Compound 168)

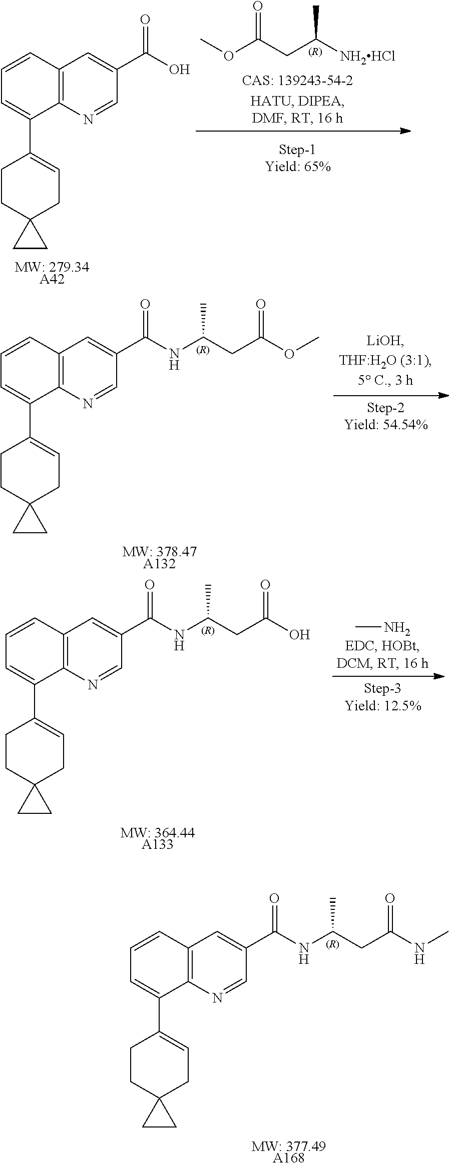

### Step-1

To a stirred solution of 8-(spiro[2.5]oct-5-en-6-yl)quinoline-3-carboxylic acid (A42) (0.3 g, 1.07 mmol, 1.0 eq.) in DMF (4.0 mL), were added HATU (0.16 g, 1.60 mmol, 1.5 eq.) and DIPEA (0.120 g, 0.32 mmol, 3.0 eq.) at 0° C. The reaction mixture was stirred under the N₂ atmosphere. After 15 min of stirring at 0° C., methyl (R)-3-aminobutanoate hydrochloride (CAS: No: 139243-54-2) (0.190 g, 1.2 mmol, 1.2 eq.) was added at same temperature and the resulting reaction mixture was stirred at 0° C. to room temperature for 16 h. TLC indicated the completion of reaction; the resulting reaction mixture was quenched with ice-cold water (30 mL) and extracted with ethyl acetate (30 mL×2). The combined organic layers were dried over Na₂SO₄, filtered and concentrated under reduced pressure to afford crude. The crude was purified by column chromatography using silica gel (60-120 Mesh) as stationary phase (100% Ethyl acetate) to afford methyl (R)-3-(8-(spiro[2.5]oct-5-en-6-yl)quinoline-3-carboxamido) butanoate (A132) (0.260 g, 0.68 mmol, Yield 65%).

¹H NMR (DMSO, 400 MHz): δ ppm, 9.24 (d, J=2.0 Hz, 1H), 8.75 (d, J=2.4 Hz, 1H), 8.68 (d, J=8.0 Hz, 1H), 7.98 (dd, J=2.0, 7.6 Hz, 1H), 7.65-7.60 (m, 2H), 5.88 (s, 1H), 4.44-4.40 (m, 1H), 3.60 (s, 3H), 2.72-2.66 (m, 3H), 2.13 (s, 2H), 1.55 (t, J=5.2 Hz, 2H), 1.24 (d, J=6.8 Hz, 3H), 0.40 (s, 4H). Note: one proton merged with DMSO solvent peak.

LCMS (Method A): 2.589 min, 100%, 254.0 nm.

### Step 2

To a stirred solution of methyl (R)-3-(8-(spiro[2.5]oct-5-en-6-yl)quinoline-3-carboxamido) butanoate (A132) (0.02 g, 0.0524 mmol, 1.0 eq.), LiOH (0.0006 g, 0.158 mmol, 3.0 eq.), in THF:H₂O (3:1) (0.3 mL), was added to the reaction mixture and stirred at room temperature for 3 h. The reaction was monitored by TLC, which indicated the completion of reaction; the resulting reaction mixture was extracted with ethyl acetate (2×30 m L). The combined organic layers dried over Na₂SO₄ and concentrated under reduced pressure to afford 0.03 g crude: (R)-3-(8-(spiro[2.5]oct-5-en-6-yl)quinoline-3-carboxamido)butanoic acid (A133) Note: crude used as such for next step without further purification.

LCMS (Method A): 2.253 min, 96.82%, 254.0 nm.

### Step 3

To a stirred solution of (R)-3-(8-(spiro[2.5]oct-5-en-6-yl)quinoline-3-carboxamido)butanoic acid (A133) (0.120 g, 0.32 mmol, 1.0 eq.) in DCM (2.0 mL), were added EDC·HCl (0.090 g, 0.49 mmol, 1.5 eq.) and HOBt (0.08 g, 0.65 mmol, 2.0 eq.) at 0° C. followed by addition of CH₃NH₂ (0.020 g, 0.65 mmol, 2.0 eq.) in DCM (2.0 mL), was added to the reaction mixture under the N₂ atmosphere. Then the resulting mixture was stirred at room temperature for 16 h. TLC indicated the completion of reaction; the resulting reaction mixture was extracted with DCM (30 mL×2). The combined organic layers dried over Na₂SO₄ and concentrated under reduced pressure to afford crude. The crude was purified by manual column chromatography using silica (60-120 Mesh) as stationary phase (30% MDC in methanol) to afford (R)—N-(4-(methylamino)-4-oxobutan-2-yl)-8-(spiro[2.5]oct-5-en-6-yl)quinoline-3-carboxamide (Compound 168) (0.015 g, 0.04 mmol, Yield: 12.5%).

¹H-NMR (MeOD, 400 MHz): δ ppm 9.22 (d, J=2.4 Hz, 1H), 8.76 (d, J=1.6 Hz, 1H), 7.94 (dd, J=1.6, 8.0 Hz, 1H), 7.69-7.61 (m, 2H), 5.89 (s, 1H), 4.65-4.53 (m, 1H), 2.75 (s, 3H), 2.67 (br d, J=1.6 Hz, 2H), 2.62-2.57 (m, 1H), 2.51-2.46 (m, 1H) 2.19 (d, J=3.2 Hz, 2H), 1.67 (t, J=6.0 Hz, 2H), 1.36 (d, J=6.8 Hz, 3H), 1.31 (s, 1H), 0.44 (s, 4H).

1H-NMR (DMSO-d₆, 400 MHz): δ ppm, 9.24 (d, J=2.0 Hz, 1H), 8.76 (d, J=2.0 Hz, 1H), 8.63 (d, J=8.0 Hz, 1H), 7.98 (d, J=5.6, 1H), 7.87 (br s, 1H), 7.65-7.59 (m, 2H), 5.88 (s, 1H), 4.41-4.39 (m, 1H), 2.67 (br s, 2H), 2.62-2.57 (m, 4H), 2.13 (s, 2H), 1.55 (t, J=6.0 Hz, 2H), 1.19 (d, J=6.8 Hz, 3H), 0.40 (s, 4H).

LCMS (Method A): 2.205 min, 98.46%, 254.0 nm

HPLC (Method A): 8.00 min, 100%, 254.0 nm

Chiral HPLC (Method): 4.53 min, 98.27%, 242.0 nm

### Example 160—Synthesis of N-((1H-pyrazol-3-yl)methyl)-8-(spiro[2.5]oct-5-en-6-yl)quinoline-3-carboxamide (Compound 169)

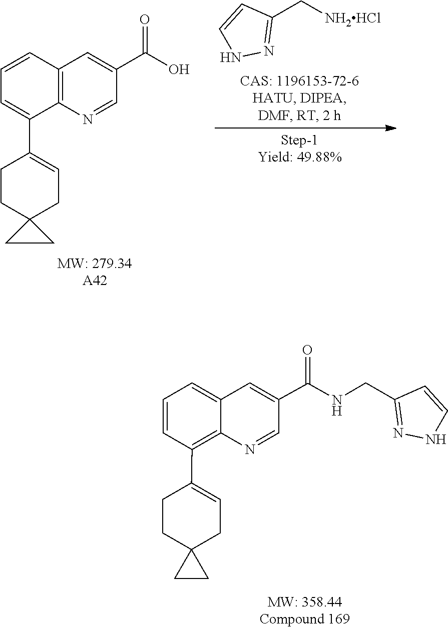

### Step-1

To a stirred solution of 8-(spiro[2.5]oct-5-en-6-yl)quinoline-3-carboxylic acid (A42) (0.1 g, 0.35 mmol, 1.0 eq.) in DMF (2.0 mL) was added DIPEA (0.138 g, 1.07 mmol, 3.0 eq.) and HATU (0.271 g, 0.71 mmol, 2.0 eq.) at 0° C. The reaction mixture was stirred under the N₂ atmosphere. After 15 min of stirring at 0° C., (1H-pyrazol-3-yl) methenamine hydrochloride (CAS: No: 1196153-72-6) (0.071 g, 0.53 mmol, 1.5 eq.) was added at same temperature and the resulting reaction mixture was stirred at 0° C. to room temperature for 2 h. TLC indicated the completion of reaction; the resulting reaction mixture was quenched with ice-cold water (20 mL) and extracted with ethyl acetate (30 mL×2). The combined organic layers were dried over Na₂SO₄, filtered and concentrated under reduced pressure to afford crude. The crude was purified by column chromatography using silica gel (45-50% Ethyl acetate in Hexane) to afford N-((1H-pyrazol-3-yl)methyl)-8-(spiro[2.5]oct-5-en-6-yl)quinoline-3-carboxamide (Compound 169) (0.064 g, 0.17 mmol, Yield: 49.88%).

¹H NMR (DMSO-d₆, 400 MHz): δ ppm, 12.65 (br s, 1H), 9.29 (d, J=1.6 Hz, 1H), 9.24 (s, 1H), 8.82 (d, J=1.6 Hz, 1H), 7.97 (d, J=6.4 Hz, 1H), 7.65-7.59 (m, 3H), 6.23 (s, 1H), 5.87 (s, 1H), 4.55 (d, J=4.8 Hz, 2H), 2.68 (s, 2H), 2.13 (s, 2H), 1.56-1.53 (t, J=6.0 Hz, 2H), 0.39 (s, 4H).

¹H NMR (MeOD, 400 MHz): δ ppm, 9.27 (d, J=2.0 Hz, 1H), 8.76 (d, J=2.4 Hz, 1H), 7.93-7.91 (dd, J=1.6, 8.0 Hz, 1H), 7.68-7.60 (m, 3H), 6.37 (d, J=2.4 Hz, 1H), 5.88 (s, 1H), 4.70 (s, 2H), 2.66 (d, J=1.6 Hz, 2H), 2.18 (d, J=2.8 Hz, 2H), 1.66 (t, J=6.0 Hz, 2H), 0.44 (s, 4H).

LCMS (UC02_FAR1): 2.178 min, 98.61%, 254.0 nm, MS: ES+359.3 (M+1).

HPLC (HP04-BR1): 8.22 min, 98.47%, 210.0 nm.

### Example 161—Synthesis of(S)—N-(1-hydroxy-3-methoxypropan-2-yl)-8-(spiro[2.5]oct-5-en-6-yl)quinoline-3-carboxamide (Compound 170)

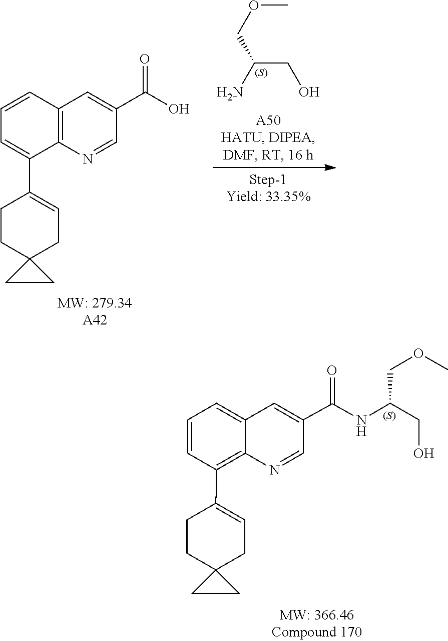

### Step-1

To a solution of 8-(spiro[2.5]oct-5-en-6-yl)quinoline-3-carboxylic acid (A42) (0.08 g, 0.28 mmol, 1.0 eq.) in DMF (2.0 mL) were added HATU (0.163 g, 0.42 mmol, 1.5 eq.) and DIPEA (0.15 mL, 0.859 mmol, 3.0 eq.) at 0° C. The reaction mixture was stirred under the N₂ atmosphere. After 15 min of stirring at 0° C., (S)-2-amino-3-methoxypropan-1-ol (A50) (0.060 g, 0.573 mmol, 2.0 eq.) was added at same temperature and the resulting reaction mixture was stirred at 0° C. to room temperature for 16 h. TLC indicated the completion of reaction; the resulting reaction mixture was quenched with ice-cold water (10 mL) and extracted with ethyl acetate (10 mL×3). The combined organic layers were dried over Na₂SO₄, filtered and concentrated under reduced pressure to afford crude. The crude was purified by column chromatography using silica gel (230-400 mesh) as stationary phase (70% Ethyl acetate in hexane as gradient) to afford(S)—N-(1-hydroxy-3-methoxypropan-2-yl)-8-(spiro[2.5]oct-5-en-6-yl)quinoline-3-carboxamide (Compound 170) (0.035 g, 0.096 mmol, Yield: 33.35%).

¹H NMR (DMSO-d₆, 400 MHz): δ ppm, 9.28 (d, J=1.6 Hz, 1H), 8.81 (d, J=1.6 Hz, 1H), 8.50 (d, J=8.0 Hz, 1H), 7.98 (d, J=5.6 Hz, 1H), 7.64-7.60 (m, 2H), 5.89 (s, 1H), 4.83 (t, J=5.6 Hz, 1H), 4.21-4.20 (m, 1H), 3.56-3.50 (m, 4H), 3.29 (s, 3H), 2.69 (s, 2H), 2.14 (s, 2H), 1.56 (t, J=5.6 Hz, 2H), 0.40 (s, 4H).

¹H NMR (MeOD, 400 MHz): δ ppm, 9.26 (d, J=2.4 Hz, 1H), 8.78 (d, J=2.4 Hz, 1H), 7.94 (dd, J=1.6, 8.0 Hz, 1H), 7.69-7.62 (m, 2H), 5.90 (s, 1H), 4.39 (t, J=5.6 Hz, 1H), 3.79-3.77 (m, 2H), 3.69-3.63 (m, 2H), 3.43 (s, 3H), 2.68 (s, 2H), 2.19 (d, J=2.8 Hz, 2H), 1.67 (t, J=6.0 Hz, 2H), 0.45 (s, 4H).

LCMS (Method A): 1.241 min, 98.90%, 244.0 nm, MS: ES+367.3 (M+1)

HPLC (Method A): 8.11 min, 99.09%, 254.0 nm

Chiral HPLC (Method A): 3.95 min, 99.08%, 242.0 nm.

### Example 162—Synthesis of (R)—N-(1-hydroxypropan-2-yl)-6-methoxy-8-(spiro[2.5]oct-5-en-6-yl)quinoline-3-carboxamide (Compound 171)

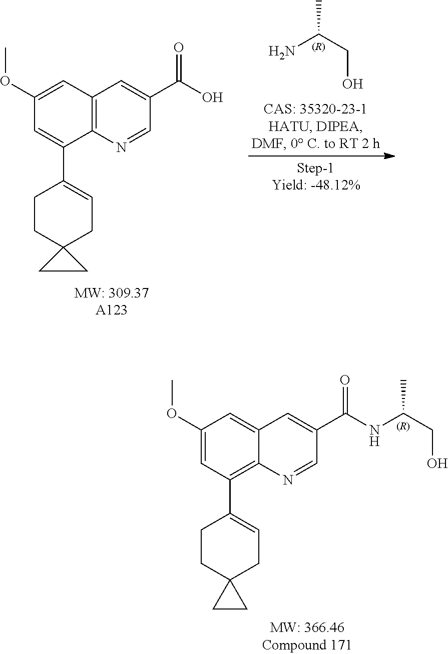

### Step-1

To a stirred solution of A123 (0.10 g, 0.323 mmol, 1.0 eq.), in DMF (2 mL), was added HATU (0.246 g, 0.647 mmol, 2.0 eq.) and DIPEA (0.125 g, 969 mmol, 3.0 eq.) respectively at 0° C. followed by addition of CAS No: 35320-23-1 (R)-2-Aminopropan-1-ol (0.036 g, 0.484 mmol, 1.5 eq.) and reaction mixture was maintained at RT for 2 h. TLC indicated the completion of reaction; the resulting reaction mixture was diluted with ice water (20 mL) and extracted with EtOAc (3×50 mL). The combined organic layers were dried over Na₂SO₄ and concentrated under reduced pressure to afford crude. The crude was purified by flash column chromatography (desired product was eluted in 50% Ethyl acetate in Hexane) to afford (R)—N-(1-hydroxypropan-2-yl)-6-methoxy-8-(spiro[2.5]oct-5-en-6-yl)quinoline-3-carboxamide (Compound 171) (0.057 g, 0.155 mmol, Yield: 48.12%).

¹H NMR (DMSO-d₆, 400 MHz): δ ppm, 9.10 (br s, 1H), 8.66 (br s, 1H), 8.40 (d, J=7.6 Hz, 1H), 7.36 (br s, 1H), 7.23 (br s, 1H), 5.90 (br s, 1H), 4.78 (t, J=5.6 Hz, 1H), 4.08-4.04 (m, 1H), 3.90 (s, 3H), 3.51-3.47 (m, 1H), 2.67 (br s, 2H), 2.12 (br s, 2H), 1.55 (br s, 2H), 1.17 (d, J=6.4 Hz, 3H), 0.39 (s, 4H),

¹H NMR (MeOD, 400 MHz): δ ppm, 9.07 (br s, 1H), 8.64 (br s, 1H), 7.27 (br s, 2H), 5.89 (br s, 1H), 4.29-4.25 (m, 1H), 3.97 (br s, 3H), 3.66 (br s, 2H), 2.64 (s, 2H), 2.17 (br s, 2H), 1.65 (t, J=6 Hz, 2H), 1.31 (d, J=6.4 Hz, 3H), 0.44 (s, 4H)

LCMS (Method-A): 2.26, 96.58%, 254 nm, MS: ES+367 (M), (M+1)

HPLC (Method-A): 8.17, 98.62%, 254 nm.

### Example 163—Synthesis of N-((1H-pyrazol-4-yl)methyl)-6-methoxy-8-(spiro[2.5]oct-5-en-6-yl)quinoline-3-carboxamide (Compound 172)

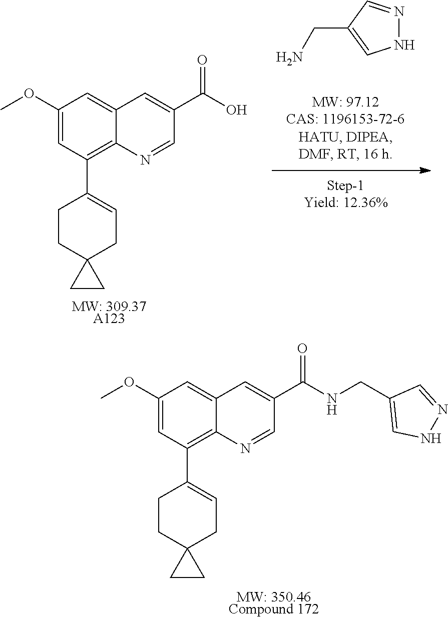

### Step-1

To a stirred solution of 6-methoxy-8-(spiro[2.5]oct-5-en-6-yl)quinoline-3-carboxylic acid (A123) (0.100 g, 0.323 mmol, 1.0 eq.) in DMF (10 mL), was added (1H-pyrazol-4-yl) methanamine CAS: 1196153-72-6 (0.047 g, 0.323 mmol, 1.0 eq.), DIPEA (0.125 g, 0.969 mmol, 3.0 eq.) and HATU (0.184 g, 0.484 mmol, 1.5 eq.). The resulting mixture was stirred at room temperature for 16 h. TLC indicated the completion of reaction; the resulting reaction mixture was diluted with water (50 mL) and extracted by EtOAc (2×50 mL). Combined organic phase was dried over Na₂SO₄ and concentrated under reduced pressure to afford crude. The obtained crude material was purified by flash column chromatography using silica gel (100-200 mesh) as stationary phase (desired product eluted in 26% EtOAc in Hexane) to afford N-((1H-pyrazol-4-yl)methyl)-6-methoxy-8-(spiro[2.5]oct-5-en-6-yl)quinoline-3-carboxamide (Compound 172) (0.014 g, 0.039 mmol, 12.36%)

¹H NMR (DMSO-d₆, 400 MHz): δ ppm, 12.62 (br s, 1H), 9.18 (br s, 1H), 9.12 (d, J=2.4 Hz, 1H), 8.69 (d, J=2.4 Hz, 1H), 7.66 (br s, 1H), 7.36 (d, J=2.8 Hz, 1H), 7.23 (d, J=2.8 Hz, 1H), 6.23 (br s, 1H), 5.90 (s, 1H), 4.53 (br s, 2H), 3.90 (s, 3H), 2.67 (br s, 2H), 2.15 (br s, 2H), 1.54 (t, J=6.0 Hz, 2H), 0.39 (s, 4H).

HPLC (Method A): 8.374 min, 96.51%, 254.0 nm.

LCMS (Method A): 2.342 min, 97.30%, 254.0 nm, MS: ES+389.4 (M+1)

### Example 164—Synthesis of (R)-6-methoxy-N-(1-(oxazol-2-yl)ethyl)-8-(spiro[2.5]oct-5-en-6-yl)quinoline-3-carboxamide (Compound 173)

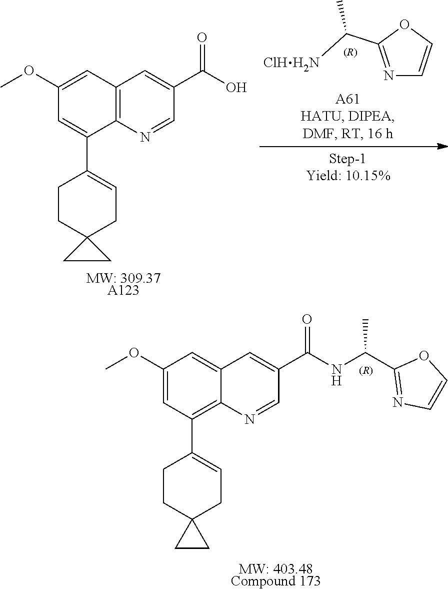

### Step-1

To a stirred solution of (A123) 6-methoxy-8-(spiro[2.5]oct-5-en-6-yl)quinoline-3-carboxylic acid (0.1 g, 3.21 mmol, 1.0 eq.) and (A61) (0.05 g, 0.38 mmol, 1.2 eq.), in DMF (4 mL), HATU (0.18 g, 4.8 mmol, 1.5 eq.) and DIPEA (0.16 mL, 0.96 mmol, 3.0 eq.), was added to the reaction mixture under N₂ atmosphere. The resulting mixture was stirred at room temperature for 16 h. TLC indicated the completion of reaction; water (20 mL) was added and extracted with ethyl acetate (2×30 mL). The combined organic layers were dried over Na₂SO₄ and concentrated under reduced pressure to afford crude. The crude was purified by manual column chromatography using silica (100-200 mesh) as stationary phase (desired product was eluted in 50% EtOAc:Hexane) to afford (R)-6-methoxy-N-(1-(oxazol-2-yl)ethyl)-8-(spiro[2.5]oct-5-en-6-yl)quinoline-3-carboxamide (Compound 173) (0.013 g, 0.032 mmol, Yield: 10.15%) 1H NMR (MeOD, 400 MHz): δ ppm 9.09 (d, J=2.0 Hz, 1H), 8.68 (d, J=2.4 Hz, 1H), 7.92 (s, 1H), 7.29 (s, 2H), 7.18 (s, 1H), 5.90 (br s, 1H), 5.49-5.47 (m, 1H), 3.97 (s, 3H), 2.65 (d, J=1.6 Hz, 2H), 2.18 (d, J=3.2 Hz, 2H), 1.72 (d, J=7.2 Hz, 3H), 1.66 (t, J=6.0 Hz, 2H), 0.40 (s, 4H).

¹H NMR (DMSO, 400 MHz): δ ppm 9.29 (d, J=7.6 Hz, 1H), 9.11 (d, J=2.0 Hz, 1H), 8.71 (d, J=2.0 Hz, 1H), 8.08 (s, 1H), 7.37 (d, J=2.8 Hz, 1H), 7.24 (d, J=2.8 Hz, 1H), 7.18 (s, 1H), 5.91 (br s, 1H), 5.38-5.35 (m, 1H), 3.91 (s, 2H), 2.67-2.50 (m, 2H), 2.12 (br s, 2H), 1.61-1.52 (m, 4H), 0.39 (s, 3H).

LCMS—2.50 min, 98.37%, 254 nm

HPLC—9.25 min, 99.23%, 254 nm

### Example 165-Synthesis of(S)—N-(1-hydroxy-3-methoxypropan-2-yl)-6-methoxy-8-(spiro[2.5]oct-5-en-6-yl)quinoline-3-carboxamide (Compound 174)

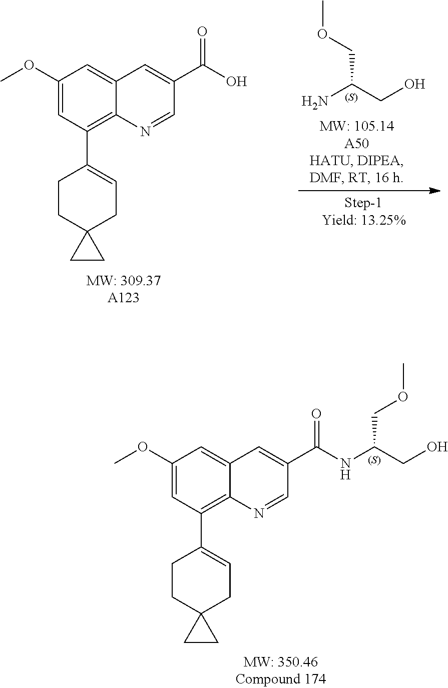

### Step-1

To a stirred solution of 6-methoxy-8-(spiro[2.5]oct-5-en-6-yl)quinoline-3-carboxylic acid (A123) (0.100 g, 0.323 mmol, 1.0 eq.) in DMF (10 mL), was added(S)-2-amino-3-methoxypropan-1-ol (A50) (0.047 g, 0.323 mmol, 1.0 eq.), DIPEA (0.125 g, 0.969 mmol, 3.0 eq.) and HATU (0.176 g, 0.465 mmol, 1.5 eq.). The resulting mixture was stirred at room temperature for 16 h. TLC indicated the completion of reaction; the resulting reaction mixture was diluted with water (50 mL) and extracted by EtOAc (2×50 mL). Organic phase was dried over Na₂SO₄ and concentrated under vacuum to afford crude. The obtained crude material was purified by flash column chromatography using silica gel (100-200 mesh) as stationary phase (desired product eluted in 7% MeOH in DCM) to afford(S)—N-(1-hydroxy-3-methoxypropan-2-yl)-6-methoxy-8-(spiro[2.5]oct-5-en-6-yl)quinoline-3-carboxamide (Compound 174) (0.015 g, 0.042 mmol, 13.25%)

¹H NMR (DMSO-d₆, 400 MHz): δ ppm, 9.10 (d, J=2.0 Hz, 1H), 8.70 (d, J=2.0 Hz, 1H), 8.47 (d, J=8.0 Hz, 1H), 7.36 (d, J=2.8 Hz, 1H), 7.24 (d, J=2.8 Hz, 1H), 5.91 (br s, 1H), 4.21-4.16 (m, 1H), 3.94 (s, 3H), 3.56-3.47 (m, 4H), 3.29 (s, 3H), 2.67 (br s, 2H), 2.12 (br s, 2H), 1.54 (t, J=6.0 Hz, 2H), 0.39 (s, 4H), 1H is merging in solvent

¹H NMR (MeOD, 400 MHz): δ ppm, 9.10 (d, J=2.4 Hz, 1H), 8.83 (br s, 1H), 7.38-7.36 (m, 2H), 5.95 (br s, 1H), 4.45-4.35 (m, 1H), 3.99 (s, 3H), 3.80-3.76 (m, 2H), 3.75-3.61 (m, 2H), 3.44 (s, 3H), 2.63 (br s, 2H), 2.20 (br s, 2H), 1.68 (t, J=6.0 Hz, 2H), 0.46 (s, 4H).

HPLC (Method A): 8.263 min, 98.34%, 254.0 nm.

LCMS (Method A): 2.249 min, 96.60%, 254.0 nm, MS: 397.2 (M+1)

### Example 166—Synthesis of N-(3-(methylsulfonamido)propyl)-8-(spiro[2.5]oct-5-en-6-yl)quinoline-3-carboxamide (Compound 175)

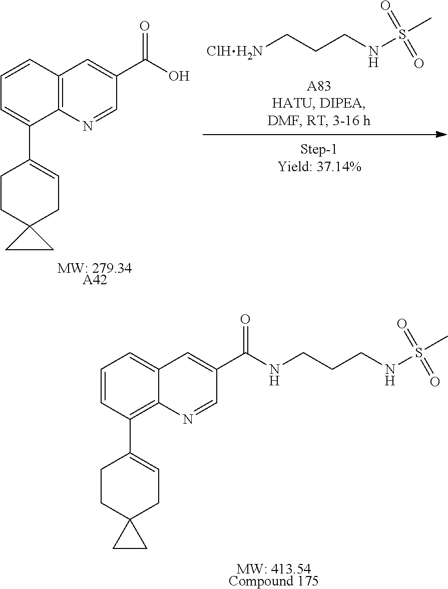

### Step-1

To a stirred solution of 8-(spiro[2.5]oct-5-en-6-yl)quinoline-3-carboxylic acid (A42) (0.100 g, 0.35 mmol, 1.0 eq.) in DMF (2.0 mL), were added HATU (0.190 g, 0.52 mmol, 1.5 eq.) and DIPEA (0.18 mL, 1.05 mmol, 3.0 eq.) at 0° C. The reaction mixture was stirred under the N₂ atmosphere. After 15 min of stirring at 0° C., N-(3-aminopropyl) methane sulfonamide hydrochloride (A83) (0.088 g, 0.46 mmol, 1.3 eq.) was added at same temperature. The resulting reaction mixture was stirred from 0° C. to room temperature for 16 h. TLC indicated the completion of reaction; the resulting reaction mixture was quenched with ice-cold water (10 mL) and extracted with EtOAc (20 mL×3). The combined organic layers were dried over Na₂SO₄ and concentrated under reduced pressure to afford crude. The crude was purified by manual column chromatography using silica gel (60-120 Mesh) as a stationary phase (10% MDC: Methanol) to afford N-(3-(methylsulfonamido)propyl)-8-(spiro[2.5]oct-5-en-6-yl)quinoline-3-carboxamide (Compound 175) (0.052 g, 0.125 mmol, Yield 37.14%)

¹H NMR (MeOD, 400 MHz): δ ppm 9.25 (d, J=2.4 Hz, 1H), 8.74 (d, J=2.0 Hz, 1H), 7.94 (dd, J=1.6, 8.0 Hz, 1H), 7.69-7.61 (m, 2H), 5.89 (s, 1H), 3.57 (t, J=6.8 Hz, 2H), 3.21 (t, J=6.8 Hz, 2H), 2.98 (s, 3H), 2.67 (br s, 2H), 2.19 (d, J=2.8 Hz, 2H), 1.96-1.89 (m, 2H), 1.67 (t, J=6.0 Hz, 2H), 0.44 (s, 4H).

¹H NMR (DMSO, 400 MHz): δ ppm 9.26 (d, J=2.4 Hz, 1H), 8.81-8.77 (m, 2H), 7.98 (dd, J=2.0, 7.6 Hz, 1H), 7.65-7.59 (m, 2H), 7.04 (t, J=5.8 Hz, 1H), 3.41-3.34 (m, 3H), 3.04 (q, J=6.4 Hz, 13.2 Hz, 2H), 2.91 (s, 3H), 2.68 (br s, 2H), 2.13 (br s, 2H), 1.78 (t, J=6.8 Hz, 2H), 1.55 (t, J=6.0 Hz, 2H), 0.40 (s, 4H).

LCMS (Method A)-2.31 min, 98.88%, 254.0 nm, MS: ES+414.3 (M+1)

HPLC (Method A)-8.28 min, 99.24%, 254.0 nm

### Example 167—Synthesis of (R)-8-(2,2-difluorospiro[3.5]non-6-en-7-yl)-N-(1-(oxazol-2-yl)ethyl)quinoline-3-carboxamide (Compound 176)

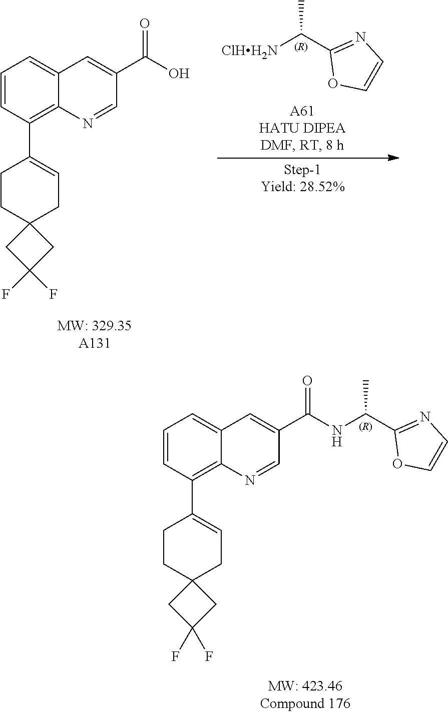

### Step-1

To a solution of 8-(2, 2-difluorospiro[3.5]non-6-en-7-yl)quinoline-3-carboxylic acid (A131) (0.060 g, 0.182 mmol, 1.0 eq.) in DMF (2.0 mL), were added DIPEA (0.19 mL, 1.093 mmol, 6.0 eq.) and HATU (0.208 g, 0.547 mmol, 3.0 eq.) at 0° C. The reaction mixture was stirred under the N₂ atmosphere. After 15 min of stirring at 0° C., (R)-1-(oxazol-2-yl)ethan-1-amine hydrochloride (A61) (0.059 g, 0.401 mmol, 2.2 eq.) was added at same temperature and the resulting reaction mixture was stirred at 0° C. to room temperature for 8 h. TLC indicated the completion of reaction; the resulting reaction mixture was quenched with ice-cold water (10 mL) and extracted with EtOAc (10 mL×3). The combined organic layers were dried over Na₂SO₄, filtered and concentrated under reduced pressure to afford crude. The crude was purified by column chromatography using silica gel (230-400 Mesh) as a stationary phase (70% Ethyl acetate in hexane as gradient) to afford (R)-8-(2,2-difluorospiro[3.5]non-6-en-7-yl)-N-(1-(oxazol-2-yl)ethyl)quinoline-3-carboxamide (Compound 176) (0.022 g, 0.052 mmol, Yield: 28.52%).

¹H NMR (DMSO-d₆, 400 MHz): δ ppm 9.34 (d, J=7.6 Hz, 1H), 9.28 (d, J=2.0 Hz, 1H), 8.84 (d, J=2.0 Hz, 1H), 8.08 (s, 1H), 7.99 (t, J=5.6 Hz, 1H), 7.63-7.60 (m, 2H), 7.19 (s, 1H), 5.79 (br s, 1H), 5.37 (t, J=7.2 Hz, 1H), 2.67 (br s, 3H), 2.38-2.33 (m, 5H), 1.85 (t, J=6.0 Hz, 2H), 1.61 (d, J=7.2 Hz, 3H).

HPLC (Method A): 8.82 min, 98.79%, 254.0 nm.

LCMS (Method A): 2.447 min, 98.19%, 254.0 nm, MS: ES+424.4 (M+1).

Chiral HPLC: 3.96 min, 97.99%, 242.0 nm.

### Example 168—Synthesis of(S)-8-(2, 2-difluorospiro[3.5]non-6-en-7-yl)-N-(1-hydroxy-3-methoxypropan-2-yl)quinoline-3-carboxamide (Compound 177)

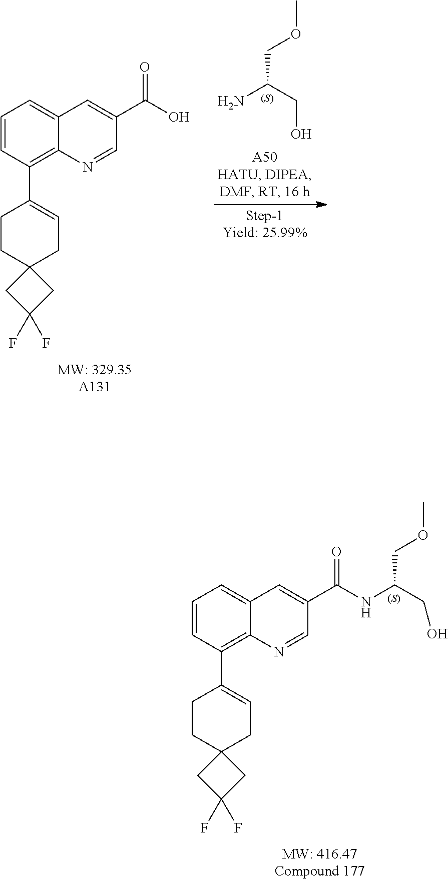

### Step-1

To a solution 8-(2, 2-difluorospiro[3.5]non-6-en-7-yl)quinoline-3-carboxylic acid (A131) (0.070 g, 0.212 mmol, 1.0 eq.) in DMF (1.0 mL), were added HATU (0.121 g, 0.319 mmol, 1.5 eq.) and DIPEA (0.10 mL, 0.636 mmol, 3.0 eq.) at 0° C. The reaction mixture was stirred under the N₂ atmosphere. After 15 min of stirring at 0° C., (S)-2-amino-3-methoxypropan-1-ol (A50) (0.044 g, 0.424 mmol, 2.0 eq.) was added at same temperature. Then the reaction mixture was stirred at room temperature for 16 h. TLC indicated the completion of reaction; the resulting reaction mixture was quenched with ice-cold water (10 mL) and extracted with EtOAc (20 mL×3). The combined organic layers were dried over Na₂SO₄ and concentrated under reduced pressure to afford crude. The obtained crude material was purified by flash column chromatography using silica gel (230-400 Mesh) as stationary phase (5% MDC in MeOH) to afford(S)-8-(2, 2-difluorospiro[3.5]non-6-en-7-yl)-N-(1-hydroxy-3-methoxypropan-2-yl)quinoline-3-carboxamide (Compound 177) (0.025 g, 0.06 mmol, Yield: 25.99%).

¹H-NMR (MeOD, 400 MHz): δ ppm, 9.25 (d, J=2.4 Hz, 1H), 8.76 (d, J=2.0 Hz, 1H), 7.94 (dd, J=2.8 Hz, 7.2 Hz, 1H), 7.65-7.62 (m, 2H), 5.80 (s, 1H), 4.40-4.37 (m, 1H), 3.77 (dd, J=1.6 Hz, 5.6 Hz, 2H), 3.66-3.64 (m, 2H), 3.42 (s, 3H), 2.68 (br s, 2H), 2.52-2.44 (m, 6H), 1.96 (t, J=6.0 Hz, 2H).

¹H-NMR (DMSO-d₆, 400 MHz): δ ppm, 9.27 (d, J=2.4 Hz, 1H), 8.81 (d, J=2.4 Hz, 1H), 8.51 (d, J=8.4 Hz, 1H), 7.98 (t, J=5.0 Hz, 1H), 7.61 (d, J=4.4 Hz, 2H), 5.79 (br s, 1H), 4.82 (t, J=5.6 Hz, 1H), 4.23-4.18 (m, 1H), 3.55-3.50 (m, 4H), 3.28 (s, 3H), 2.69 (br s, 2H), 2.46-2.38 (m, 4H), 2.33 (br s, 2H), 1.85 (t, J=6 Hz, 2H).

LCMS (Method A): 2.240 min, 100%, 254.0 nm, MS: ES+417.5 (M+1)

HPLC (Method A): 7.91 min, 100%, 254.0 nm

CHIRAL HPLC (Method A): 3.88 min, 98.79%, 260.0 nm

### BIOLOGICAL EXAMPLES

Prior art TEAD inhibitors 1-1, 1-2, 1-3, and 1-4 (structures provided in Table 3) are used as reference compounds throughout Biological Examples 169-179.

TABLE 3
Prior art TEAD inhibitors

| Compound Number | Structure |
|---|---|
| 1-1 | 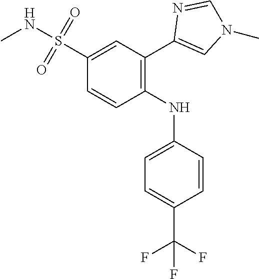 |
| 1-2 | 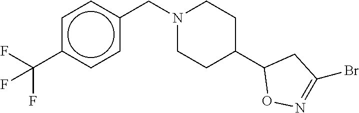 |
| 1-3 | 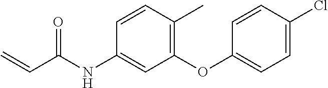 [SMILES: C=CC(=O)Nc1ccc(C)c(Oc2ccc(Cl)cc2)c1] |
| 1-4 | 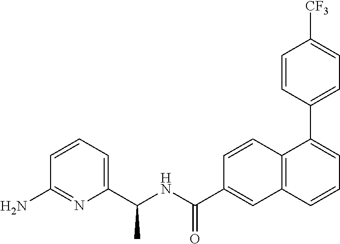 [SMILES: C[C@H](NC(=O)c1ccc2c(-c3ccc(C(F)(F)F)cc3)cccc2c1)c1cccc(N)n1] |

### Example 169—NCI-H226 and H28 Proliferation and Cell Viability Assay

Cell proliferation of a TEAD dependent NF2-null mesothelioma cell line, NCI-H226, and a TEAD-non-dependent mesothelioma cell line, NCI-H28 was assayed using two different protocols:
a) CellTiter-Glo 2.0 (Promega #G9243). The effect of the TEAD inhibitors disclosed herein on cell proliferation was determined in TEAD1-genetically dependent cell line NCI-H226 (responder cell line) and TEAD-independent cell lines (NCI-H23, HEK293T), as non-responder, negative control. Cells were seeded in 96-well plates (Thermo Scientific #167008) in growth media (RPMI-1640, Gibco #61870-010) for NCI-H226 and NCI-H23 or DMEM (Gibco #31966-021, HEK293T) with 10% Heat Inactivated Fetal Bovine Serum (Gibco 10500-064) at 800 (NCI-H226 and HEK-293T) or 2000 (NCI-H23) cells per well at 37° C., 5% CO₂, humidified incubator; 24 hours later, fresh growth media containing test compound or DMSO vehicle was added and incubated with the cells for 144 hrs. TEAD inhibitor stocks were made at a 1000× fold excess in DMSO, and a dose response test compound dose range of 10,000 to 0.1 nM was used. Each treatment was carried out in triplicate. After 144 hrs of treatment, relative cell viability numbers were determined using the CellTiter-Glo 2.0 assay following the manufacturer's instructions. Test plates and CellTiter-Glo 2.0 reagent were allowed to equilibrate to room temperature. CellTiter-Glo 2.0 reagent was added at 1:1 reagent to media ratio to the 96-well cell plates, plates were placed on a plate shaker for 2 minutes and then incubated at room temperature for a further 10 minutes to allow complete cell lysis to occur. The mix was then transferred to white, 96-well luminometer assay plates (Greiner Bio One #655075). Luminescence was quantified using a CLARIOStar Plus multi-mode plate reader (BMG Labtech) in luminescence detection mode with the recommended settings (ultra-Glo pre-set: emission 545-50 nm with autofocus and enhanced dynamic range on). Relative cell density was expressed as a function of inhibitor concentration, with respect to DMSO-only control wells. Data where plotted using GraphPad Prism and IC₅₀ values for the biological effect were calculated using the EC₅₀ non-linear fit function using a sigmoidal, 4-parameter logistic, least squares fit with no constraints.
b) Cell Titer One (Promega, #G3582). The effect of the TEAD inhibitors disclosed herein on cell proliferation was also determined using the Promega Substrate Cell Titer Aqueous One Solution Reagent. The TEAD inhibitors were evaluated for the effects on cell proliferation responder (H226) and non-responder negative control cancer cell lines (CAMA1, H28), as well as non-transformed normal human cells (HUVEC, NHDF). Dissociated cells were resuspend in media (depending on cell line, either in DMEM, RPMI, or EMEM all with 10% FBS, with gentamicin as an antibiotic). Cells were seeded at 100 μL/well at 750 cells per well in 96-well plates and allowed to settle and attach. After an incubation of 24 hrs (37° C., 5% CO₂, humidified incubator), 100 μL of test compound-containing media was added. A dilution series of 2000× concentrated stock of test compounds was prepared in DMSO, for final concentrations from 0.1 to 10,000 nM. After addition of test compound-containing media, cells were incubated for 6 days at 37° C., 5% CO₂, humidified incubators). To measure cell density, 20 μL of Promega Substrate Cell Titer 96 Aqueous One Solution Reagent (Promega, #G3582) to each well was added according to the manufacturer's instructions. Plates were incubated at 37° C. for usually 3-8 hrs, to reach in the control cells (without compound) an OD of 1.5 at 490 nm. The OD was measured using Vmax [Molecular Devices] spectrophotometer plate reader. Media only-wells were used as background controls and cell densities normalized to DMSO vehicle control. ECs were taken as the concentration achieving 50% of maximal growth inhibition.

For IC₅₀ and EC₅₀ data “A” indicates a value of less than 0.2 μM; the symbol “B” indicates a value in the range of from 0.2 μM up to 1 μM; and the symbol “C” indicates a value of greater than 1 μM. ND: no data.

TABLE 4
Cytotoxic potency measured by CTG assay for the
H226-responder and H23-non-responder cell lines

| Cmpd No. | NCI-H226 IC₅₀ | NCI-H23 IC₅₀ |
|---|---|---|
| 1 | A | C |
| 2 | A | C |
| 5 | A | C |
| 6 | A | C |
| 7 | A | C |
| 11 | A | C |
| 16 | A | ND |
| 19 | A | C |
| 20 | A | C |
| 21 | B | C |
| 22 | A | C |
| 26 | A | ND |
| 27 | A | ND |
| 29 | B | ND |
| 30 | A | ND |
| 31 | A | ND |
| 12 | A | C |
| 13 | A | C |
| 38 | A | ND |
| 39 | A | ND |
| 40 | A | ND |

TABLE 5
Cytotoxic potency measured by CTG assay
for the H226-responder cell line

| Compound | EC50 |
|---|---|
| Number | (nM) |
| 1-2 | A |
| 1-3 | A |
| 7 | A |
| 11 | A |
| 19 | A |
| 47 | A |
| 62 | A |
| 165 | A |
| 166 | A |
| 109 | A |
| 110 | A |

TABLE 6
Cytotoxic potency measured by Cell Titer
One assay for the H226 and H28 cell lines

| Cmpd No. | NCI-H226 IC₅₀ | NCI-H28 IC₅₀ |
|---|---|---|
| 1 | A | C |
| 2 | A | C |
| 5 | A | ND |
| 6 | A | C |
| 7 | A | C |
| 11 | A | C |
| 16 | A | C |
| 19 | A | C |
| 20 | A | C |
| 21 | B | C |
| 22 | B | C |
| 26 | A | ND |
| 27 | A | ND |
| 29 | C | ND |
| 30 | A | ND |
| 31 | C | ND |
| 35 | B | ND |
| 36 | C | ND |
| 37 | C | ND |
| 12 | B | ND |
| 13 | A | ND |
| 38 | B | ND |
| 39 | A | ND |
| 40 | B | ND |

### Example 170—Mero14 Proliferation and Cell Viability Assay

Mero14 cells were trypsinised, counted and plated at 400 cells/well in 200 μl of growth media in 96 well flat bottom tissue culture treated plates. The plated cells were incubated overnight at 37° C., 5% CO₂ before being treated with compounds in triplicate for each assay point. 50 μl of the serially diluted compound was added to each well giving a final DMSO concentration of 0.5% per well and a total volume of 250 μl per well. The treated cells were incubated for 144 hours (6 days) at 37° C., 5% CO₂. Following compound treatment the remaining viable cells were detected using the Promega CellTiter-Glo 2.0 assay as follows: 150 μl of media was removed from each of the wells to be assayed and 100 μl of room temperature CellTiter-Glo 2.0 reagent was added. The plate was placed on an orbital shaker at 500 rpm at room temperature for 2 minutes, and then incubated without shaking for a further 10 minutes at room temperature. 150 μl of cell lysate was removed from each well and dispensed into a single well of a white opaque 96 well plate.

Luminescence at 545 nm was read using a BMG CLARIOstar plate reader. The luminescent signal was directly proportional to number of viable cells, the percent of viable cells relative to the DMSO control was then calculated for each assay point. Percent viability relative to DMSO control is plotted against compound concentration and a curve fitted with non-linear regression in GraphPad Prism software. IC₅₀ values were calculated using GraphPad Prism software as follows: For Relative IC₅₀ values, a non-constrained four-parameter logistic curve fit was used (Sigmoidal, 4PL, X is concentration). For Absolute IC₅₀ values the baseline was constrained to Y=0 (Absolute IC₅₀, X is concentration). Area Under the Curve (AUC): Prism computed area-under-the-curve by the trapezoidal method. The analysis sees a fitted curve as simply a series of connected XY points. Prism simply computes the area under the “curve” defined by connecting the points by straight lines. The units of the AUC are the units of the Y axis times units of the X axis.

For IC₅₀ and EC₅₀ data “A” indicates a value of less than 0.2 μM; the symbol “B” indicates a value in the range of from 0.2 μM up to 1 μM; and the symbol “C” indicates a value of greater than 1 μM. ND: no data.

TABLE 7
Cytotoxic potency measured by CTG assay for Mero14 cell line

| Compound | EC50 |
|---|---|
| Number | (nM) |
| 1-2 | B |
| 1-3 | A |
| 7 | A |
| 11 | A |
| 19 | A |
| 47 | A |
| 62 | A |
| 165 | A |
| 166 | A |
| 109 | A |
| 110 | A |

### Example 171—Mero82 Proliferation and Cell Viability Assay

Mero 82 cells were trypsinised, counted and plated at 400 cells/well in 200 μl of growth media in 96 well flat bottom tissue culture treated plates. The plated cells were incubated overnight at 37° C., 5% CO₂ before being treated with compounds in triplicate for each assay point. 50 μl of the serially diluted compound was added to each well giving a final DMSO concentration of 0.5% per well and a total volume of 250 μl per well. The treated cells were incubated for 144 hours (6 days) at 37° C., 5% CO₂. Following compound treatment, the remaining viable cells were detected using the Promega CellTiter-Glo 2.0 assay as follows: 150 μl of media was removed from each of the wells to be assayed and 100 μl of room temperature CellTiter-Glo 2.0 reagent was added. The plate was placed on an orbital shaker at 500 rpm at room temperature for 2 minutes, and then incubated without shaking for a further 10 minutes at room temperature. 150 μl of cell lysate was removed from each well and dispensed into a single well of a white opaque 96 well plate. Luminescence at 545 nm was read using a BMG CLARIOstar plate reader. The luminescent signal was directly proportional to number of viable cells, the percent of viable cells relative to the DMSO control was then calculated for each assay point. Percent viability relative to DMSO control is plotted against compound concentration and a curve fitted with non-linear regression in GraphPad Prism software. IC₅₀ values were calculated using GraphPad Prism software as follows: For Relative IC₅₀ values a non-constrained four-parameter logistic curve fit was used (Sigmoidal, 4PL, X is concentration). For Absolute IC₅₀ values the baseline was constrained to Y=0 (Absolute IC₅₀, X is concentration). Area Under the Curve (AUC): Prism computes area-under-the-curve by the trapezoidal method. The analysis sees a fitted curve as simply a series of connected XY points. Prism simply computes the area under the “curve” defined by connecting the points by straight lines. The units of the AUC are the units of the Y axis times units of the X axis.

For IC₅₀ and EC₅₀ data “A” indicates a value of less than 0.2 μM; the symbol “B” indicates a value in the range of from 0.2 μM up to 1 μM; and the symbol “C” indicates a value of greater than 1 μM. ND: no data.

TABLE 8
Cytotoxic potency measured by CTG assay for Mero82 cell line

| Compound | EC50 |
|---|---|
| Number | (nM) |
| 1-2 | C |
| 1-3 | A |
| 7 | A |
| 11 | A |
| 19 | A |
| 47 | A |
| 62 | A |
| 165 | A |
| 166 | A |
| 109 | A |
| 110 | A |

### Example 172—BHY Proliferation and Cell Viability Assay

BHY cells were trypsinised, counted and plated at the specified cell density in 200 μl of growth media in 96 well flat bottom tissue culture treated plates. The plated cells were incubated overnight at 37° C., 5% CO₂ before being treated with compounds in triplicate for each assay point. 50 μl of the serially diluted compound was added to each well giving a final DMSO concentration of 0.5% per well and a total volume of 250 μl per well. The treated cells were incubated for 144 hours (6 days) at 37° C., 5% CO₂. Following compound treatment, the remaining viable cells were detected using the Promega CellTiter-Glo 2.0 assay as follows: 150 μl of media was removed from each of the wells to be assayed and 100 μl of room temperature CellTiter-Glo 2.0 reagent was added. The plate was placed on an orbital shaker at 500 rpm at room temperature for 2 minutes, and then incubated without shaking for a further 10 minutes at room temperature. 150 μl of cell lysate was removed from each well and dispensed into a single well of a white opaque 96 well plate. Luminescence at 545 nm was read using a BMG CLARIOstar plate reader. The luminescent signal was directly proportional to number of viable cells, the percent of viable cells relative to the DMSO control was then calculated for each assay point. Percent viability relative to DMSO control was plotted against compound concentration and a curve fitted with non-linear regression in GraphPad

Prism software. EC₅₀ and IC₅₀ values are calculated using GraphPad Prism software as follows: For EC₅₀ values, a non-constrained four-parameter logistic curve fit was used (Sigmoidal, 4PL, X is concentration). For Absolute IC₅₀ values the baseline was constrained to Y=0 (Absolute IC₅₀, X is concentration). Area Under the Curve (AUC): Prism computes area-under-the-curve by the trapezoidal method. The analysis sees a fitted curve as simply a series of connected XY points. Prism simply computes the area under the “curve” defined by connecting the points by straight lines. The units of the AUC are the units of the Y axis times units of the X axis.

For IC₅₀ and EC₅₀ data “A” indicates a value of less than 0.2 μM; the symbol “B” indicates a value in the range of from 0.2 μM up to 1 M; and the symbol “C” indicates a value of greater than 1 μM. ND: no data.

TABLE 9
Cytotoxic potency measured by CTG assay for BHY cell line

| Compound | EC50 |
|---|---|
| Number | (nM) |
| 1-2 | C |
| 1-3 | A |
| 7 | A |
| 19 | A |
| 20 | A |
| 47 | A |
| 62 | A |
| 81 | A |
| 82 | B |
| 109 | A |
| 103 | A |
| 104 | A |

### Example 173—LOU-NH91 Proliferation and Cell Viability Assay

LOU-NH91 cells were trypsinised, counted and plated at the specified cell density in 200 μl of growth media in 96 well flat bottom tissue culture treated plates. The plated cells were incubated overnight at 37° C., 5% CO₂ before being treated with compounds in triplicate for each assay point. 50 μl of the serially diluted compound was added to each well giving a final DMSO concentration of 0.5% per well and a total volume of 250 μl per well. The treated cells were incubated for 216 hours (9 days) at 37° C., 5% CO₂. Following compound treatment, the remaining viable cells were detected using the Promega CellTiter-Glo 2.0 assay as follows: 150 μl of media was removed from each of the wells to be assayed and 100 μl of room temperature CellTiter-Glo 2.0 reagent was added. The plate was placed on an orbital shaker at 500 rpm at room temperature for 2 minutes, and then incubated without shaking for a further 10 minutes at room temperature. 150 μl of cell lysate was removed from each well and dispensed into a single well of a white opaque 96 well plate. Luminescence at 545 nm was read using a BMG CLARIOstar plate reader. The luminescent signal was directly proportional to number of viable cells, the percent of viable cells relative to the DMSO control was then calculated for each assay point. Percent viability relative to DMSO control was plotted against compound concentration and a curve fitted with non-linear regression in GraphPad Prism software. EC₅₀ and IC₅₀ values were calculated using GraphPad Prism software as follows: For EC₅₀ values a non-constrained four-parameter logistic curve fit was used (Sigmoidal, 4PL, X is concentration). For Absolute IC₅₀ values the baseline was constrained to Y=0 (Absolute IC₅₀, X is concentration). Area Under the Curve (AUC): Prism computes area-under-the-curve by the trapezoidal method. The analysis sees a fitted curve as simply a series of connected XY points. Prism simply computes the area under the “curve” defined by connecting the points by straight lines. The units of the AUC are the units of the Y axis times units of the X axis.

For IC₅₀ and EC₅₀ data “A” indicates a value of less than 0.2 μM; the symbol “B” indicates a value in the range of from 0.2 μM up to 1 M; and the symbol “C” indicates a value of greater than 1 μM. ND: no data.

TABLE 10
Cytotoxic potency measured by CTG
assay for LOU-NH91 cell line

| Compound | EC50 |
|---|---|
| Number | (nM) |
| 1-2 | A |
| 1-3 | A |
| 7 | A |
| 19 | A |
| 20 | A |
| 47 | A |
| 62 | A |
| 81 | A |
| 82 | A |
| 109 | A |
| 103 | A |
| 104 | A |

### Example 174—MSTO-211H Proliferation and Cell Viability Assay

MSTO-211H cells were trypsinised, counted and plated at the specified cell density in 200 μl of growth media in 96 well flat bottom tissue culture treated plates. The plated cells were incubated overnight at 37° C., 5% CO₂ before being treated with compounds in triplicate for each assay point. 50 μl of the serially diluted compound was added to each well giving a final DMSO concentration of 0.5% per well and a total volume of 250 μl per well. The treated cells were incubated for 144 hours (6 days) at 37° C., 5% CO₂. Following compound treatment, the remaining viable cells were detected using the Promega CellTiter-Glo 2.0 assay as follows: 150 μl of media was removed from each of the wells to be assayed and 100 μl of room temperature CellTiter-Glo 2.0 reagent was added. The plate was placed on an orbital shaker at 500 rpm at room temperature for 2 minutes, and then incubated without shaking for a further 10 minutes at room temperature. 150 μl of cell lysate was removed from each well and dispensed into a single well of a white opaque 96 well plate. Luminescence at 545 nm is read using a BMG CLARIOstar plate reader. The luminescent signal was directly proportional to number of viable cells, the percent of viable cells relative to the DMSO control was then calculated for each assay point. Percent viability relative to DMSO control was plotted against compound concentration and a curve fitted with non-linear regression in GraphPad Prism software. IC₅₀ values were calculated using GraphPad Prism software as follows: For Relative IC₅₀ values a non-constrained four-parameter logistic curve fit was used (Sigmoidal, 4PL, X is concentration). For Absolute IC₅₀ values the baseline was constrained to Y=0 (Absolute IC₅₀, X is concentration). Area Under the Curve (AUC): Prism computes area-under-the-curve by the trapezoidal method. The analysis sees a fitted curve as simply a series of connected XY points. Prism simply computes the area under the “curve” defined by connecting the points by straight lines. The units of the AUC are the units of the Y axis times units of the X axis.

For IC₅₀ and EC₅₀ data “A” indicates a value of less than 0.2 μM; the symbol “B” indicates a value in the range of from 0.2 M up to 1 μM; and the symbol “C” indicates a value of greater than 1 μM. ND: no data.

TABLE 11
Cytotoxic potency measured by CTG
assay for MSTO-211H cell line

| Compound | EC50 |
|---|---|
| Number | (nM) |
| 1-2 | A |
| 1-3 | A |
| 7 | A |
| 11 | A |
| 19 | A |
| 47 | A |
| 62 | A |
| 165 | A |
| 166 | A |
| 109 | A |
| 110 | A |

### Example 175—SDM103T2 Proliferation and Cell Viability Assay

SDM103T2 cells were trypsinised, counted and plated at the specified cell density in 200 μl of growth media in 96 well flat bottom tissue culture treated plates. The plated cells were incubated overnight at 37° C., 5% CO₂ before being treated with compounds in triplicate for each assay point. 50 μl of the serially diluted compound was added to each well giving a final DMSO concentration of 0.5% per well and a total volume of 250 μl per well. The treated cells were incubated for 144 hours (6 days) at 37° C., 5% CO₂. Following compound treatment the remaining viable cells were detected using the Promega CellTiter-Glo 2.0 assay as follows: 150 μl of media was removed from each of the wells to be assayed and 100 μl of room temperature CellTiter-Glo 2.0 reagent was added. The plate was placed on an orbital shaker at 500 rpm at room temperature for 2 minutes, and then incubated without shaking for a further 10 minutes at room temperature. 150 μl of cell lysate was removed from each well and dispensed into a single well of a white opaque 96 well plate. Luminescence at 545 nm was read using a BMG CLARIOstar plate reader. The luminescent signal was directly proportional to number of viable cells, the percent of viable cells relative to the DMSO control was then calculated for each assay point. Percent viability relative to DMSO control was plotted against compound concentration and a curve fitted with non-linear regression in GraphPad Prism software. EC₅₀ and IC₅₀ values were calculated using GraphPad Prism software as follows: For EC₅₀ values a non-constrained four-parameter logistic curve fit was used (Sigmoidal, 4PL, X is concentration). For Absolute IC₅₀ values the baseline was constrained to Y=0 (Absolute IC₅₀, X is concentration). Area Under the Curve (AUC): Prism computes area-under-the-curve by the trapezoidal method. The analysis sees a fitted curve as simply a series of connected XY points. Prism simply computes the area under the “curve” defined by connecting the points by straight lines. The units of the AUC are the units of the Y axis times units of the X axis.

For IC₅₀ and EC₅₀ data “A” indicates a value of less than 0.2 μM; the symbol “B” indicates a value in the range of from 0.2 M up to 1 μM; and the symbol “C” indicates a value of greater than 1 μM. ND: no data.

TABLE 12
Cytotoxic potency measured by CTG
assay for SDM103T2 cell line

| Compound | EC50 |
|---|---|
| Number | (nM) |
| 1-1 | A |
| 1-2 | C |
| 1-3 | A |
| 7 | A |
| 11 | A |
| 19 | A |
| 47 | A |
| 62 | A |
| 165 | A |
| 166 | A |
| 109 | A |
| 110 | A |

### Example 176—Cell-Based TEAD-Promoter Transcriptional Activity Assay

Luciferase based TEAD-transcriptional cell-based reporter assay: To measure the effect of the TEAD inhibitors disclosed herein on TEAD transcriptional activity in cell-based systems, we employed a stable MCF7 cell line expressing luciferase under a consensus synthetic TEAD/YAP promoter element (MCF7-Luc, BPS Bioscience, Catalog #60618). This MCF7-TEAD-Luc cell line has been extensively validated with both genetic and pharmacological intervention, showing inhibition of luciferase expression and luminescence in response to ablation of the TEAD/YAP pathway. MCF7-Luc cells were plated at 10,000 cells per well in white 384-well plates and settled by a quick spin. The media was MEM (Gibco) supplemented with 10% fetal bovine serum (ExCell), selenium-transferrin-insulin mix (Gibco), 1× non-essential amino acids (Gibco) and sodium pyruvate (Gibco). Cells were allowed to attach and grow overnight (37° C., 5% CO₂ in humidified incubator), and test compounds were added the next day. Compounds were dissolved at 1000× fold excess in DMSO and made into 2-fold serial dilution, and added to media at 30 μL of media per well. A DMSO-only well serves a vehicle control. The highest concentration of drug in media was 1,000 nM. Positive control compounds include K975 (pan-TEAD inhibitor) and negative control (Actinomycin). Cells were incubated for 24 hrs with test compounds and positive controls, after which time, the luminescence was measured by the One-Glo luciferase kit (Promega). 30 μL of One-Glo reagent was added to each well and shaken for 8 minutes to lyse the cells. The plates were centrifugated at 1000 RPM for 1 minute to settle all liquid in the well and eliminate bubbles. Luminescence was quantified using an ENVision plate reader (Perkin Elmer). Percent inhibition of luminescence by test compound was expressed as a function of test compound concentration, with the no-test compound DMSO only wells serving as baseline. EC₅₀ values for each compound were calculated with a non-linear asymptotic line fit.

For EC₅₀ data “A” indicates a value of less than 0.2 μM; the symbol “B” indicates a value in the range of from 0.2 μM up to 1 μM; and the symbol “C” indicates a value of greater than 1 μM.

TABLE 13
MCF-7 TEAD Luciferase Assay

| Cmpd No. | MCF-7 Luc EC₅₀ |
|---|---|
| 1 | A |
| 2 | A |
| 11 | A |
| 19 | A |
| 20 | A |

### Example 177—TEAD Thermal Shift Assay (TSA)

Thermal shift assay (TSA) is a biophysical method to evaluate stability of proteins with change in temperature. The assay measures thermal denaturation temperature in varying conditions such as different drug concentration. For this example, fluorogenic dye was used that bind to unfolded proteins. SYPRO Orange binds non-specifically to hydrophobic surfaces, and water strongly quenches its fluorescence. When the protein unfolds, the exposed hydrophobic surfaces bind the dye, resulting in an increase in fluorescence by excluding water. Detergent micelles also bind the dye and increase background noise dramatically. Binding of small molecules to proteins leads to a stabilization against to thermal denaturation with the degree of thermal stabilization proportional to the strength of binding.

The study described in this example investigated the change in melting temperature (ΔTm) of TEAD isoforms 1˜4 when treated with TEAD inhibitors. Compound 1-1-a known strong TEAD1 and weak TEAD2 inhibitor; as well as Compound 1-4-a pan-TEAD inhibitor were used as controls. As expected, Compound 1-1 showed pronounced thermal shift only for TEAD1 whilst Compound 1-4 showed thermal shifts for all four TEAD isoforms. As shown in Table 14, compounds of the disclosure uniformly displayed TEAD1 binding and significantly divergent binding to TEAD2-4 Experimental Parameters/Conditions

| Parameters | Conditions |
|---|---|
| Assay Format | Biophysical, 96-well Bio-Rad plate |
| Protein | Human TEADs |
| Sypro Orange | 5X Conc. |
| DMSO | 1% |
| Ramp rate | 1° C./min |
| Assay Buffer | 20 mM Tris-HCL (pH: 8), 200 mM NaCl, 2 mM |
| | TCEP, 10% Glycerol, 0.01% Tween 20 |
| Assay Volume | 25 μL |
| Assay Readout | Melt Curve 20-95° C. |
| Instrument | Bio-Rad family of CFX Real-time PCR systems |

### Assay Buffer Preparation

| Component | | | Final | | Weighed | | Total |
|---|---|---|---|---|---|---|---|
| Name | Source | Cat # | Conc. | | quantity | | Volume |
| Trizma | Sigma | T5941- | 20 | mM | 31.52 | mg | 10 mL |
| hydrochloride | | 500G | | | | | |
| NaCl | Sd fine- | 20241 | 200 | mM | 116.88 | mg | |
| | chem ltd | | | | | | |
| TCEP | Sigma | C4706-2G | 2 | mM | 5.73 | mg | |
| Glycerol | Sigma | G5516- | 10% | | 1 | mL | |
| | | 100ML | | | | | |
| Tween 20 | Sigma | P9416- | 0.01% | | 1 | μL | |
| | | 100ML | | | | | |
| Cell culture | Hi-media | TCL019- | — | | — | | |
| grade water | | 500ML | | | | | |

Tris-HCL was dissolved in 5 mL of Cell culture grade water and allowed to dissolve completely. The pH was adjusted to 8 using NaOH. Additional assay buffer components were added in the order listed above. The buffer was mixed slowly.

### Assay Flow

Assay buffer was prepared according to the procedures outlined above and dispensed into test wells. Spyro orange was added to a concentration of 5× in the assay buffer, followed by test compound stock solution which was added to a concentration of 10 μM or 30 μM in the assay buffer.

The melt cycle was performed in Bio-RAD PCR instrument from 20-95° C. with ramp rate of 1° C./min.

Thermal shift data are provided in Table 14.

TABLE 14
Thermal shift assay data

| | Ratio of Protein to | TEAD1 | TEAD2 | TEAD3 | TEAD4 |
|---|---|---|---|---|---|
| Compound | Compound | ΔTm(° C.) | | | |
| 1-1 | 1:1 | 2 | 1 | 0 | 0 |
| 1-1 | 1:3 | 8 | 1 | 0 | 1 |
| 1-4 | 1:1 | 10 | 5 | 5 | 5 |
| 1-4 | 1:3 | 12 | 7 | 9 | 9 |
| 2 | 1:1 | 10 | 1 | 2 | 2 |
| 2 | 1:3 | 12 | 1.5 | 3 | 4 |
| 7 | 1:1 | 9 | 2 | 2 | 1.5 |
| 7 | 1:3 | 11 | 3 | 2 | 3 |
| 11 | 1:1 | 11 | 3 | 3.5 | 1.5 |
| 11 | 1:3 | 11.5 | 3 | 3 | 2.5 |
| 19 | 1:1 | 12 | 2 | 2 | 2 |
| 19 | 1:3 | 12 | 3 | 4 | 3 |
| 20 | 1:1 | 10 | 1 | 2 | 1.5 |
| 20 | 1:3 | 13 | 3 | 4.5 | 3 |
| 47 | 1:1 | 9 | 2 | 2 | 3 |
| 47 | 1:3 | 11 | 2 | 3 | 3 |
| 62 | 1:1 | 9 | 3 | 1 | 2 |
| 62 | 1:3 | 11 | 3 | 2 | 3 |
| 81 | 1:1 | 9 | 2 | 4 | 5 |
| 81 | 1:3 | 10 | 3 | 5 | 7 |
| 109 | 1:1 | 13 | 3 | 2 | 1 |
| 109 | 1:3 | 14 | 2 | 3 | 2 |
| 110 | 1:1 | 14 | 2 | 2 | 2 |
| 110 | 1:3 | 16 | 3 | 3 | 3 |

### Example 178—Inhibition of mouse xenograft MSTO-211H tumors with TEAD inhibitors

This study evaluated the in vivo anti-tumor efficacy of test compounds in BALB/c nude mice for treatment of MSTO-211H xenograft of lung cancer.

### Abbreviations

AAALAC Association for Assessment and Accreditation of Laboratory Animal Care
BW Body Weight
g gram
IACUC Institutional Animal Care and Use Committee
kg kilogram
mg milligram
mL milliliter
mm millimeter
PO Per Os
R&D Research and Development
RT Room Temperature
RTV Relative Tumor Volume
SEM Standard Error of the Mean
TGI Tumor Growth Inhibition
TV Tumor Volume
TW Tumor Weight
UL microliter
dd H₂O double distilled water
T/C Relative tumor proliferation rate
AA Antibiotic-Antimycotic
CMC-Na Carbokyl Methyl Cellulose-Na
BID Bis in Die
Tween 80 Tween 80

### Animals and Housing Conditions

Species: Mus Musculus
Strain: BALB/c nude
Age: 6-8 weeks
Sex: female
Body weight: 18-22 g
Number of animals inoculated with tumor: 80 mice
Number of animals enrolled in the treatment: 36 mice
Number of unenrolled animals: 44 mice
Animal supplier: Beijing Vital River Biotechnology Experimental Animal Co. Ltd
   Certification number: 20170011010591

### Study Design

| | | | | Dosing | | | |
|---|---|---|---|---|---|---|---|
| | | | Dosage | Volume^{b} | Dosing | Dosing | Dosing |
| Group | N^{a} | Compound | (mg/kg) | (μL/g) | route | Frequency^{c} | Schedule^{c} |
| 1 | 4 | Vehicle | — | 10 | PO | BID | 27 |
| 3 | 4 | 7 | 12.5 | 10 | PO | BID | 27 |
| 5 | 4 | 110 | 12.5 | 10 | PO | BID | 27 |
| 7 | 4 | 166 | 12.5 | 10 | PO | BID | 27 |
| 8 | 4 | 109 | 12.5 | 10 | PO | BID | 27 |
| 9 | 4 | 62 | 12.5 | 10 | PO | BID | 27 |

Note:
^{a}N, animal number in each group.
^{b}Dose volume: adjust dose volume based on body weight 10 μL/g.
^{c}Treatment schedule was adjusted if body weight loss >15%.

### Cell Culture

The MSTO-211H tumor cells were maintained in RPMI 1640 medium supplemented with 10% Fetal bovine serum and 1% Antibiotic-Antimycotic at 37° C. in an atmosphere of 5% CO₂ in air. The tumor cells were routinely subcultured twice weekly by trypsin-EDTA. The cells growing in an exponential growth phase with 95% cell survival or above were harvested, and counted for tumor inoculation.

### Tumor Cell Information

| Cell lines | Cancer Type | Species | Vendor | Cat. No. |
|---|---|---|---|---|
| MSTO-211H | Lung cancer | human | ATCC | CRL-2081 |

### Tumor Inoculation

Each mouse was inoculated subcutaneously at the right flank with MSTO-211H tumor cells (5×10⁶+50% matrigel/0.2 mL) for tumor development. Animals were marked with earrings with unique 6 significant figure number for identification. The treatments were started when the average tumor volume reached an average size of 181 mm³.

### Tumor Measurement

The major endpoint was to see if the tumor growth could be delayed or mice could be cured. Tumor volume was measured twice a week in two dimensions using a caliper, and the volume was expressed in mm³ using the formula: V=0.5 a×b² where a and b are the long and short diameters of the tumor, respectively. The tumor size was then used for calculations of T/C value. The TGI value (in percent) or the T/C value (in percent) is an indication of antitumor effectiveness; T and C are the mean tumor volumes or tumor weights of the treated and control groups, respectively, on a given day.

TGI was calculated for each group using the formula: TGI (%)=[1−(Tᵢ−T₀)/(Vᵢ−V₀)]×100; Ti is the average tumor volume of a treatment group on a given day, To is the average tumor volume of the treatment group on the day of treatment start, Vᵢ is the average tumor volume of the vehicle control group on the same day with Tᵢ, and V₀ is the average tumor volume of the vehicle group on the day of treatment start.

Relative T/C (%)=T_{RTV}/C_{RTV}×100 (RTV(%)=Vₜ/V₀×100, Vₜ is the average tumor volume on a given day, V₀ is the average tumor volume on the first day of treatment. T_{RTV} is the RTV of a treatment group, and the C_{RTV} is the RTV of the vehicle control group.)

### Statistical Analysis

Summary statistics, including mean and the standard error of the mean (SEM), were provided for the tumor volume of each group at each time point.

Statistical analysis of difference in tumor volume among the groups was conducted on the data obtained at the end of the study.

One-tailed T test was performed to compare tumor volumes between two groups. All data are analyzed using GraphPad Prism 6.0. p<0.05 is considered to be statistically significant.

The antitumor effectiveness of the study was analyzed on PG-D27 when vehicle group reached 588 mm³.

### Tumor Volume Trace

The change in tumor volume over the course of the study is provided in Table 15.

TABLE 15
Tumor size over course of study.

| | | Days after the start of treatment^{a} | | | | | | | | |
|---|---|---|---|---|---|---|---|---|---|---|
| Gr. | Treatment | 0 | 3 | 6 | 10 | 13 | 17 | 20 | 23 | 27 |
| 1 | Vehicle, PO, BID | 181 ± | 249 ± | 287 ± | 327 ± | 363 ± | 392 ± | 431 ± | 489 ± | 588 ± |
| | | 5 | 23 | 32 | 48 | 43 | 51 | 51 | 54 | 73 |
| 3 | Compound 7, 12.5 | 181 ± | 245 ± | 219 ± | 124 ± | 99 ± | 86 ± | 109 ± | 117 ± | 126 ± |
| | mg/kg, PO, BID | 12 | 31 | 29 | 8 | 7 | 6 | 12 | 20 | 16 |
| 5 | Compound 110, 12.5 | 181 ± | 207 ± | 179 ± | 107 ± | 75 ± | 51 ± | 42 ± | 53 ± | 50 ± |
| | mg/kg, PO, BID | 9 | 11 | 24 | 16 | 9 | 5 | 4 | 8 | 2 |
| 7 | Compound 166, 12.5 | 181 ± | 192 ± | 177 ± | 153 ± | 145 ± | 139 ± | 148 ± | 184 ± | 219 ± |
| | mg/kg, PO, BID | 7 | 11 | 36 | 18 | 8 | 25 | 21 | 26 | 25 |
| 8 | Compound 109, 12.5 | 181 ± | 216 ± | 174 ± | 89 ± | 59 ± | 44 ± | 44 ± | 46 ± | 58 ± |
| | mg/kg, PO, BID | 11 | 21 | 18 | 5 | 3 | 3 | 4 | 4 | 8 |
| 9 | Compound 62, 12.5 | 181 ± | 224 ± | 228 ± | 149 ± | 111 ± | 82 ± | 88 ± | 101 ± | 104 ± |
| | mg/kg, PO, BID | 10 | 14 | 30 | 14 | 2 | 9 | 9 | 7 | 3 |

Note:
^{a}Mean ± SEM

### Tumor Growth Inhibition Analysis

Tumor growth inhibition rate for the test compounds was calculated based on tumor volume measurement at day 27 after the start of treatment. The T/C values calculated according to tumor volume are close to each other, and the trend is shown in Table 16.

TABLE 16
Tumor size on day 27.

| | | Tumor | | | P value |
|---|---|---|---|---|---|
| | | Volume | T/CD^{b} | TGI^{b} | compared |
| Gr. | Treatment | (mm³)^{a} | (%) | (%) | With vehicle^{c} |
| 1 | Vehicle , | 588 ± | — | — | — |
| | PO, BID | 73 | | | |
| 3 | Compound | 126 ± | 21.4 | 113.6 | p < 0.001 |
| | 7, 12.5 | 16 | | | |
| | mg/kg, PO, | | | | |
| | BID | | | | |
| 5 | Compound | 50 ± | 8.5 | 132.4 | p < 0.001 |
| | 110, 12.5 | 2 | | | |
| | mg/kg, PO, | | | | |
| | BID | | | | |
| | Compound | 219 ± | 38.1 | 90.7 | p < 0.01 |
| 7 | 166, 12.5 | 25 | | | |
| | mg/kg, PO, | | | | |
| | BID | | | | |
| 8 | Compound | 58 ± | 10.1 | 130.2 | p < 0.001 |
| | 109, 12.5 | 8 | | | |
| | mg/kg, PO, | | | | |
| | BID | | | | |
| 9 | Compound | 104 ± | 17.9 | 119.1 | p < 0.001 |
| | 62, 12.5 | 3 | | | |
| | mg/kg, PO, | | | | |
| | BID | | | | |

^{a}Mean + SEM.
^{b}T/C(%) = TRTV / CRTV × 100, TGI = [1 − (Tᵢ − T₀)/ (Vᵢ − V₀)] × 100.
^{c}One-tailed T test analysis was performed to compare tumor volumes between two groups.

### Example 179—Inhibition of Mouse Xenograft NCI-H226 Tumors with TEAD Inhibitors

This study evaluated the in vivo therapeutic efficacy of TEAD inhibitors in the treatment of the subcutaneous lung cancer NCI-H226 xenograft model in female BALB/c nude mice.

### Abbreviations

ANOVA Analysis of variance
BW Body weight
BWL Body weight loss
FFPE Formalin fixed paraffin embedded
IACUC Institutional animal care and use committee
MTV Mean tumor volume
N/A Not available or not applicable
p.o. Oral (ly)
QD Once a day
REG REG=[(T0−Ti)/T0]×100%;
   Ti as the mean tumor volume of the treatment group on the measurement day;
   T0 as the mean tumor volume of the treatment group on Day 0.
ROA Route of administration
RT Room temperature
RTV Relative tumor volume
SD Standard deviation
SEM Standard error of mean
SOC Standard of care used in clinical setting for a specific disease
TBD To be determined
T/C The Ti/Ci value (%): T/C %=Ti/Ci×100;
   Ti and Ci are the mean tumor volumes of the treatment and control groups, respectively, on a given day.
TGI Tumor growth inhibition; TGI %=(1−Ti/Ci)×100;
   Ti and Ci are the mean tumor volumes of the treatment and control groups, respectively, on a given day.
TV Tumor volume
PK/PD Pharmacokinetics/Pharmacodynamics
ΔInhibition Mean % ΔInhibition=((Ci−C0)−(Ti−T0))/(Ci−C0)*100%
   Ti and Ci are the mean tumor volumes of the treatment and control groups, respectively, on a given day. T0 and C0 as the mean tumor volumes of the treatment and vehicle groups on Day 0.

### Animal and Model

Species Mus musculus
Strain Balb/c nude
Supplier GemPharmatech Co., Ltd
Gender Female
No. of Animals Ordered 68 (45 plus 23 spare)
No. of Animals Assigned 45
to Study
Average Age of Animals 6-8 weeks
at Study Initiation

### Tumor Cell Information

| Cell line | Cancer Type |
|---|---|
| NCI-H226 | Lung cancer |

### Study Design

| | | | Dose | Dosing | Dosing | | Dosing |
|---|---|---|---|---|---|---|---|
| | No. of | | level | Solution | Volume | | Frequency |
| Group | mice | Compound | (mg/kg) | (mg/ml) | (mL/kg) | ROA | & Duration |
| 1 | 5 | Vehicle | — | — | 10 | p.o. | QD × 28 days |
| 2 | 5 | No treatment | | | | | QD × 28 days |
| 3 | 5 | 1-1 | 3 | 0.3 | 10 | p.o. | QD × 28 days |
| 4 | 5 | 2 | 25 | 2.5 | 10 | p.o. | QD × 28 days |
| 5 | 5 | 2 | 10 | 1 | 10 | p.o. | QD × 28 days |
| 6 | 5 | 2 | 3 | 0.3 | 10 | p.o. | QD × 28 days |
| 7 | 5 | 11 | 25 | 2.5 | 10 | p.o. | QD × 28 days |
| 8 | 5 | 11 | 10 | 1 | 10 | p.o. | QD × 28 days |
| 9 | 5 | 11 | 3 | 0.3 | 10 | p.o. | QD × 28 days |

### Cell Culture

The NCI-H226 cells (CL-00364) cancer cells were maintained in vitro with RPMI1640 medium supplemented with 10% fetal bovine serum at 37° C. in an atmosphere of 5% CO₂ in the air. The cells in exponential growth phase were harvested and quantitated by cell counter before tumor inoculation.

### Tumor Inoculation

Each mouse was inoculated subcutaneously in the right front flank region with NCI-H226 tumor cells (1×10⁷) in 0.2 ml of PBS mixed with Matrigel (1:1) for tumor development.

### Randomization

The randomization was performed when the mean tumor size reached 138.81 mm³. 45 mice were enrolled in the study. All animals were randomly allocated to 9 study groups, 5 mice in each group. Randomization was performed based on “Matched distribution” method/“Stratified” method (StudyDirector™ software, version 3.1.399.19)/randomized block design. The date of randomization was denoted as day 0.

### Test Compound Administration

The treatment was initiated on the same day of randomization (day 0) per study design.

### Observation and Data Collection

After tumor cells inoculation, the animals were checked daily for morbidity and mortality. During routine monitoring, the animals were checked for any effects of tumor growth and treatments on behavior such as mobility, food and water consumption, body weight gain/loss (Body weights were measured twice per week after randomization), eye/hair matting and any other abnormalities. Mortality and observed clinical signs were recorded for individual animals in detail.

Tumor volumes were measured twice per week after randomization in two dimensions using a caliper, and the volume was expressed in mm³ using the formula: “V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet.

The body weights and tumor volumes were measured by using StudyDirector™ software (version 3.1.399.19).

### Statistical Analysis

To compare tumor volumes of different groups at a pre-specified day, Bartlett's test was first used to check the assumption of homogeneity of variance across all groups. When the p-value of Bartlett's test was >=0.05, one-way ANOVA was performed to test overall equality of means across all groups. If the p-value of the one-way ANOVA was <0.05, post hoc testing was performed by running Tukey's HSD (honest significant difference) tests for all pairwise comparisons, and Dunnett's tests for comparing each treatment group with the vehicle group. When the p-value of Bartlett's test was <0.05, Kruskal-Wallis test was performed to test overall equality of medians among all groups. If the p-value the Kruskal-Wallis test was <0.05, post hoc testing was performed by running Conover's non-parametric test for all pairwise comparisons or for comparing each treatment group with the vehicle group, both with single-step p-value adjustment.

In addition, pairwise comparisons were performed without multiple comparison correction and report nominal/uncorrected p-values directly from Welch's t-test or Mann-Whitney U test. Specifically, Bartlett's test was first used to check the assumption of homogeneity of variance for a pair of groups. When the p-value of Bartlett's test was ≥0.05, Welch's t-test was performed; otherwise Mann-Whitney U test was performed, to obtain nominal p-values.

All statistical analyses were performed in R-a language and environment for statistical computing and graphics (version 3.3.1). All tests were two-sided unless otherwise specified, and p-values of <0.05 were regarded as statistically significant.

### Results—Tumor Growth Inhibition

TABLE 17
TGI and T/C calculation on Day 27

| | | Dose | Day 27 | | | |
|---|---|---|---|---|---|---|
| | | Level | Tumor Volume | T/C | TGI | |
| Group | Compound | (mg/kg) | (mm³, mean ± SEM) | (%) | (%) | P Value |
| 1 | Vehicle | — | 534.18 ± 57.34 | — | — | — |
| 2 | No | — | 636.87 ± 179.23 | 119.22 | −19.22 | 1 |
| | treatment | | | | | |
| 3 | 1-1 | 3 | 66.85 ± 12.17 | 12.51 | 87.49 | 5.49e−07 |
| 4 | 2 | 25 | 105.03 ± 13.17 | 19.66 | 80.34 | 3.12e−05 |
| 5 | 2 | 10 | 243.78 ± 74.70 | 45.64 | 54.36 | 0.1 |
| 6 | 2 | 3 | 345.21 ± 85.33 | 64.62 | 35.38 | 0.605 |
| 7 | 11 | 25 | 112.73 ± 17.20 | 21.10 | 78.90 | 6.14e−05 |
| 8 | 11 | 10 | 194.94 ± 42.44 | 36.49 | 63.51 | 0.0201 |
| 9 | 11 | 3 | 479.84 ± 96.60 | 89.83 | 10.17 | 1 |

Note:
Bartlett's test was performed to test homogeneity of variance and normality, P = 4.09e−06, significant, non-parametric test was used to compare groups. P < 0.05 was considered to be statistically significant.

What is claimed is:

1. A compound having the chemical formula (I): 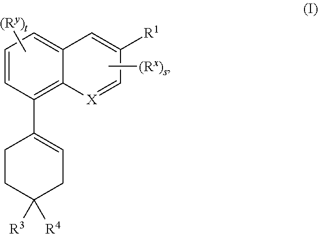
or a pharmaceutically acceptable salt thereof, wherein:
X is N or CH;
R¹ is selected from the group consisting of —C(O)OR⁵, —C(O)—NR⁶R², —S(O)₂—N(R⁶)₂, —S(O)ₘ—(C₁₋₆alkyl), and —S(O)N(R⁶)₂;
R² is selected from the group consisting of —C₁-C₆ alkyl, —(C₁-C₆ alkyl substituted with one or two —OR⁵), —(C₁-C₆ alkylene)-CN, —(C₁-C₆ alkylene)-S(O)ₙ—(C₁-C₆ alkyl), —(C₁-C₄ alkylene optionally substituted with OR⁵)—C(O)OR⁵, —(C₁-C₄ alkylene optionally substituted with OR⁵)—C(O)N(R⁶)₂, —(C₀-C₄ alkylene)-phenyl, —(C₁-C₆ alkylene)-N(R⁶)₂, 5-6 membered heteroaryl having one, two, or three heteroatoms each independently selected from N, O, and S, —(C₁-C₄ alkylene optionally substituted with OR⁵)-(5-6 membered heteroaryl having one, two, or three heteroatoms each independently selected from N, O, and S), —(C₀-C₄ alkylene)-C₃-C₁₀cycloalkyl, and —(C₀-C₄ alkylene)-(3-10 membered heterocyclyl having one, two, three, or four heteroatoms each independently selected from N, O, and S), wherein any aforementioned phenyl, 5-6 membered heteroaryl, C₃-C₁₀cycloalkyl, and 3-10 membered heterocyclyl are optionally substituted;
m is 1 or 2;
n is 0, 1, or 2;
R³ is selected from the group consisting of hydrogen, halogen, —C₁-C₆ alkyl, —(C₁-C₆ haloalkyl), —O—(C₁-C₆ alkyl), and —O—(C₁-C₆ haloalkyl);
R⁴ is selected from the group consisting of hydrogen, halogen, and —C₁-C₆ alkyl; or
R³ and R⁴ are taken together to form a 3-7 membered carbocyclic ring with the carbon to which R³ and R⁴ are attached, wherein the carbocyclic ring is optionally substituted with one or more halogens;
each R⁵ is independently hydrogen or —C₁-C₆ alkyl;
each R⁶ is independently hydrogen or —C₁-C₆ alkyl;
each R^{x} is independently selected from the group consisting of —C₁-C₆ alkyl, halogen, —OR⁵, —CN, and —N(R⁶)²;
each R^{y} is independently selected from the group consisting of —C₁-C₆ alkyl, halogen, —OR⁵, —CN, and —N(R⁶)₂;
s is 0, 1, or 2; and
t is 0, 1, 2, or 3.

2. A compound having the chemical formula (I′): 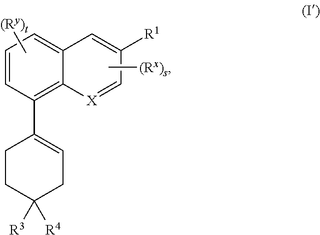
or a pharmaceutically acceptable salt thereof, wherein:
X is N or CH;
R¹ is selected from the group consisting of —C(O)OR⁵, —C(O)—NR⁶R², —S(O)₂—N(R⁶)₂, —S(O)ₘ—(C₁₋₆alkyl), and —S(O)N(R⁶)₂;
R² is selected from the group consisting of —C₁-C₆ alkyl, —(C₁-C₆ alkyl substituted with one or two —OR⁵), —(C₁-C₆ alkylene)-CN, —(C₁-C₆ alkylene)-S(O)ₙ—(C₁-C₆ alkyl), —(C₁-C₄ alkylene optionally substituted with OR⁵)—C(O)OR⁵, —(C₁-C₄ alkylene optionally substituted with CN or OR⁵)—C(O)N(R⁶)₂, —(C₀-C₄ alkylene)-phenyl, —(C₁-C₆ alkylene)-N(R⁶R⁷), —(C₁-C₆ alkylene)-OP(O)(OR⁵)₂, 5-6 membered heteroaryl having one, two, or three heteroatoms each independently selected from N, O, and S, —(C₁-C₄ alkylene optionally substituted with OR⁵)-(5-6 membered heteroaryl having one, two, or three heteroatoms each independently selected from N, O, and S), —(C₀-C₄ alkylene)-C₃-C₁₀cycloalkyl, and —(C₀-C₄ alkylene)-(3-10 membered heterocyclyl having one, two, three, or four heteroatoms each independently selected from N, O, and S), wherein any aforementioned phenyl, 5-6 membered heteroaryl, C₃-C₁₀cycloalkyl, and 3-10 membered heterocyclyl are optionally substituted with one or more R^{w};
m is 1 or 2;
n is 0, 1, or 2;
R³ is selected from the group consisting of hydrogen, halogen, —C₁-C₆ alkyl, —(C₁-C₆ haloalkyl), —O—(C₁-C₆ alkyl), and —O—(C₁-C₆ haloalkyl);
R⁴ is selected from the group consisting of hydrogen, halogen, and —C₁-C₆ alkyl; or
R³ and R⁴ are taken together to form a 3-7 membered carbocyclic ring with the carbon to which R³ and R⁴ are attached, wherein the carbocyclic ring is optionally substituted with one or more halogens;
each R⁵ is independently hydrogen or —C₁-C₆ alkyl;
each R⁶ is independently hydrogen or —C₁-C₆ alkyl;
R⁷ is selected from the group consisting of hydrogen, —C₁-C₆ alkyl, —C(O)—(C₁₋₆ alkyl), —C(O)N(R⁶)₂, —C(O)₂—(C₁₋₆ alkyl), —S(O)ₙ—(C₁-C₆ alkyl), and —S(O)ₙNR⁶—(C₁-C₆ alkyl);
each R^{w} is independently selected from the group consisting of —C₁-C₆ alkyl, halogen, —N(R⁶)₂, and oxo, wherein the —C₁-C₆ alkyl is optionally substituted with —OH;
each R^{x} is independently selected from the group consisting of —C₁-C₆ alkyl, halogen, —OR⁵, and —CN;
each R^{y} is independently selected from the group consisting of —C₁-C₆ alkyl, halogen, —OR⁵, and —CN;
s is 0, 1, or 2; and
t is 0, 1, 2, or 3.

3. The compound of claim 1 or 2, wherein X is N.

4. The compound of any one of claims 1-3, wherein R¹ is —C(O)—NHR₂.

5. The compound of any one of claims 1-3, wherein R¹ is —C(O)OH.

6. The compound of any one of claims 1-3, wherein R¹ is —S(O)CH₃.

7. The compound of any one of claims 1-3, wherein R¹ is —S(O)₂CH₃.

8. The compound of any one of claims 1-3, wherein R¹ is —S(O)₂NHCH₃.

9. The compound of any one of claims 1-8, wherein R² is selected from the group consisting of —C₁-C₆ alkyl, —(C₁-C₆ alkyl substituted with one or two —OR⁵), —(C₁-C₆ alkylene)-CN, —(C₁-C₆ alkylene)-S(O)ₙ—(C₁-C₆ alkyl), —(C₁-C₄ alkylene optionally substituted with OR⁵)—C(O)OR⁵, —(C₁-C₄ alkylene optionally substituted with OR⁵)—C(O)N(R⁶)₂, —(C₁-C₆ alkylene)-N(R⁶)₂, and —(C₁-C₄ alkylene optionally substituted with OR⁵)-(5-6 membered heteroaryl having one, two, or three heteroatoms each independently selected from N, O, and S).

10. The compound of any one of claims 1-9, wherein R² is —(C₁-C₄ alkylene)-5-6 membered heteroaryl.

11. The compound of any one of claims 1-10, wherein R² is —CH(CH₃)-5-6 membered heteroaryl.

12. The compound of any one of claims 1-10, wherein R² is —CH₂-5-6 membered heteroaryl.

13. The compound of any one of claims 1-12, wherein the 5-6 membered heteroaryl is optionally substituted with C₁-C₆alkyl or N(R^{a})₂, wherein each R^{a} is independently hydrogen or C₁-C₆alkyl.

14. The compound of any one of claims 1-13, wherein the 5-6 membered heteroaryl is pyridyl.

15. The compound of any one of claims 1-14, wherein the 5-6 membered heteroaryl is

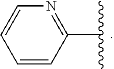 [SMILES: *c1ccccn1]

16. The compound of any one of claims 1-13, wherein the 5-6 membered heteroaryl is oxazolyl.

17. The compound of any one of claims 1-13 and 16, wherein the 5-6 membered heteroaryl is

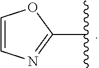 [SMILES: *c1ncco1]

18. The compound of any one of claims 1-9, wherein R² is —C₁-C₆ alkyl.

19. The compound of any one of claims 1-9 and 18, wherein R² is isopropyl.

20. The compound of any one of claims 1-9, wherein R² is —(C₁-C₆ alkyl substituted with one or two —OR⁵).

21. The compound of any one of claims 1-9, wherein R² is —(C₁-C₆ alkyl substituted with —OR⁵).

22. The compound of any one of claims 1-9, 20, and 21, wherein R² is —(C₁-C₆ alkyl substituted with —OH).

23. The compound of any one of claims 1-9 and 20, wherein R² is —(C₁-C₆ alkyl substituted with —OH and —OCH₃).

24. The compound of any one of claims 1-9, wherein R² is —(C₁-C₆ alkylene)-S(O)ₙ—(C₁-C₆ alkyl).

25. The compound of any one of claims 1-9, wherein R² is —(C₁-C₆ alkylene)-CN.

26. The compound of any one of claims 1-9, wherein R² is —(C₁-C₄ alkylene optionally substituted with OR⁵)—C(O)OR⁵.

27. The compound of any one of claims 1-9, wherein R² is —(C₁-C₄ alkylene optionally substituted with OR⁵)—C(O)N(R⁶)₂.

28. The compound of any one of claims 1-9, wherein R² is —(C₁-C₆ alkylene)-N(R⁶)₂.

29. The compound of any one of claims 1-28, wherein R³ is —(C₁-C₆ haloalkyl).

30. The compound of any one of claims 1-29, wherein R³ is trifluoromethyl.

31. The compound of any one of claims 1-28, wherein R³ is halogen (e.g., —F).

32. The compound of any one of claims 1-31, wherein R⁴ is hydrogen.

33. The compound of any one of claims 1-31, wherein R⁴ is halogen (e.g., —F).

34. The compound of any one of claims 1-28, wherein R³ and R⁴ are taken together to form a 3-7 membered carbocyclic ring with the carbon to which R³ and R⁴ are attached, wherein the carbocyclic ring is optionally substituted with one or more halogens (e.g., —F).

35. The compound of any one of claims 1-34, wherein s and t are both 0.

36. A compound of formula (Ia): 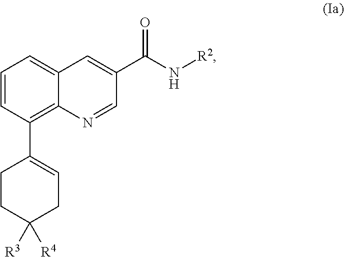
or a pharmaceutically acceptable salt thereof, wherein:
R² is selected from the group consisting of —C₁-C₆ alkyl, —(C₁-C₆ alkyl substituted with one or two —OR⁵), —(C₁-C₆ alkylene)-CN, —(C₁-C₆ alkylene)-S(O)ₙ—(C₁-C₆ alkyl), —(C₁-C₄ alkylene optionally substituted with OR⁵)—C(O)OR⁵, —(C₁-C₄ alkylene optionally substituted with OR⁵)—C(O)N(R⁶)₂, —(C₁-C₆ alkylene)-N(R⁶)₂, and —(C₁-C₄ alkylene optionally substituted with OR⁵)-(5-6 membered heteroaryl having one, two, or three heteroatoms each independently selected from N, O, and S), wherein the 5-6 membered heteroaryl is optionally substituted;
n is 0, 1, or 2;
R³ is selected from the group consisting of halogen, —C₁-C₆ alkyl, —C₁-C₆ haloalkyl, and —O—(C₁-C₆ alkyl);
R⁴ is selected from the group consisting of hydrogen, halogen, and —C₁-C₆ alkyl; or
R³ and R⁴ are taken together to form a 3-7 membered carbocyclic ring with the carbon to which R³ and R⁴ are attached, wherein the 3-7 membered carbocyclic ring is optionally substituted with one or more halogens;
each R⁵ is independently for each occurrence, hydrogen or —C₁-C₆ alkyl; and
each R⁶ is independently for each occurrence, hydrogen or —C₁-C₆ alkyl.

37. A compound of formula (Ia′): 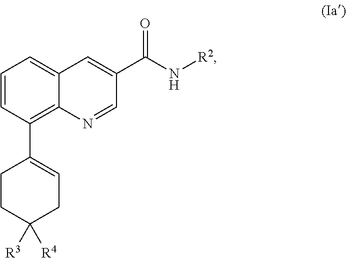
or a pharmaceutically acceptable salt thereof, wherein:
R² is selected from the group consisting of —C₁-C₆ alkyl, —(C₁-C₆ alkyl substituted with one or two —OR⁵), —(C₁-C₆ alkylene)-CN, —(C₁-C₆ alkylene)-S(O)ₙ—(C₁-C₆ alkyl), —(C₁-C₄ alkylene optionally substituted with CN or OR⁵)—C(O)OR⁵, —(C₁-C₄ alkylene optionally substituted with OR⁵)—C(O)N(R⁶)₂, —(C₁-C₆ alkylene)-N(R⁶R⁷), —(C₁-C₆ alkylene)-OP(O)(OR⁵)₂, and —(C₁-C₄ alkylene optionally substituted with OR⁵)-(5-6 membered heteroaryl having one, two, or three heteroatoms each independently selected from N, O, and S), wherein the 5-6 membered heteroaryl is optionally substituted with one or more R^{w};
n is 0, 1, or 2;
R³ is selected from the group consisting of halogen, —C₁-C₆ alkyl, —C₁-C₆ haloalkyl, and —O—(C₁-C₆ alkyl);
R⁴ is selected from the group consisting of hydrogen, halogen, and —C₁-C₆ alkyl; or
R³ and R⁴ are taken together to form a 3-7 membered carbocyclic ring with the carbon to which R³ and R⁴ are attached, wherein the 3-7 membered carbocyclic ring is optionally substituted with one or more halogens;
each R⁵ is independently for each occurrence, hydrogen or —C₁-C₆ alkyl; and
each R⁶ is independently for each occurrence, hydrogen or —C₁-C₆ alkyl
R⁷ is selected from the group consisting of hydrogen, —C₁-C₆ alkyl, —C(O)—(C₁₋₆ alkyl), —C(O)N(R⁶)₂, —C(O)₂—(C₁₋₆ alkyl), —S(O)ₙ—(C₁-C₆ alkyl), and —S(O)ₙNR⁶—(C₁-C₆ alkyl);
each R⁷ is independently selected from the group consisting of —C₁-C₆ alkyl, —N(R⁶)₂, and oxo, wherein the —C₁-C₆ alkyl is optionally substituted with —OH.

38. The compound of claim 36 or 37, wherein the compound is a compound of formula (Ib): 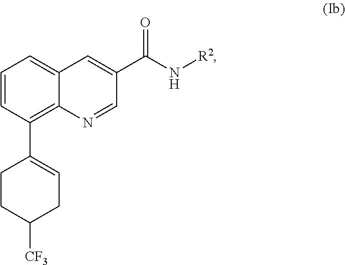
or a pharmaceutically acceptable salt thereof.

39. The compound of claim 36 or 37, wherein the compound is a compound of formula (Ic): 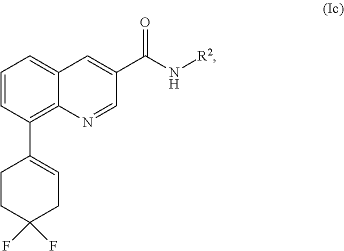
or a pharmaceutically acceptable salt thereof.

40. The compound of any one of claims 36-39, wherein R² is —(C₁-C₄ alkylene)-5-6 membered heteroaryl.

41. The compound of any one of claims 36-39, wherein R² is —CH(CH₃)-5-6 membered heteroaryl.

42. The compound of any one of claims 36-39, wherein R² is —CH₂-5-6 membered heteroaryl.

43. The compound of any one of claims 36-42, wherein the 5-6 membered heteroaryl is optionally substituted with —C₁-C₆alkyl or —N(R^{a})², wherein each R^{a} is independently hydrogen or C₁-C₆alkyl.

44. The compound of any one of claims 36-43, wherein the 5-6 membered heteroaryl is pyridyl.

45. The compound of any one of claims 36-44, wherein the 5-6 membered heteroaryl is

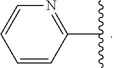 [SMILES: *c1ccccn1]

46. The compound of any one of claims 36-43, wherein the 5-6 membered heteroaryl is oxazolyl.

47. The compound of any one of claims 36-43 and 46, wherein the 5-6 membered heteroaryl is

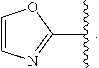 [SMILES: *c1ncco1]

48. The compound of any one of claims 37-39, wherein R² is selected from the group consisting of —(C₁-C₆ alkyl substituted with one or two —OR⁵), —(C₁-C₆ alkylene)-S(O)ₙ—(C₁-C₆ alkyl), —(C₁-C₄ alkylene optionally substituted with OR⁵)—C(O)OR⁵, —(C₁-C₄ alkylene optionally substituted with OR⁵)—C(O)N(R⁶)₂, —(C₁-C₆ alkylene)-N(R⁶R⁷)₂, —(C₁-C₆ alkylene)-OP(O)(OR⁵)₂, (C₁-C₄ alkylene optionally substituted with OR⁵)-(5-6 membered heteroaryl having one, two, or three heteroatoms each independently selected from N, O, and S), and —(C₀-C₄ alkylene)-(3-10 membered heterocyclyl having one, two, three, or four heteroatoms each independently selected from N, O, and S), wherein any aforementioned 5-6 membered heteroaryl and 3-10 membered heterocyclyl are optionally substituted with 1, 2, 3, or 4 substituents each independently selected from the group consisting of methyl, —NH₂, and oxo.

49. The compound of any one of claims 37-39, wherein R² is selected from the group consisting of —(C₁-C₆ alkyl substituted with one or two —OR⁵), —(C₁-C₆ alkylene)-N(R⁶R⁷)₂, and —(C₁-C₄ alkylene optionally substituted with OR⁵)-(5-6 membered heteroaryl having one, two, or three heteroatoms each independently selected from N, O, and S), wherein any aforementioned 5-6 membered heteroaryl and 3-10 membered heterocyclyl are optionally substituted with 1, 2, 3, or 4 substituents each independently selected from the group consisting of methyl, —NH₂, and OXO.

50. The compound of any one of claims 36-39, wherein R² is —C₁-C₆ alkyl.

51. The compound of any one of claims 36-39 and 50, wherein R² is isopropyl.

52. The compound of any one of claims 36-39, wherein R² is —(C₁-C₆ alkyl substituted with one or two —OR⁵).

53. The compound of any one of claims 36-39 and 52, wherein R² is —(C₁-C₆ alkyl substituted with —OR⁵).

54. The compound of any one of claims 36-39, 52, and 53, wherein R² is —(C₁-C₆ alkyl substituted with —OH).

55. The compound of any one of claims 36-39, wherein R² is —(C₁-C₆ alkyl substituted with —OH and —OCH₃).

56. The compound of any one of claims 36-39, wherein R² is —(C₁-C₆ alkylene)-S(O)ₙ—(C₁-C₆ alkyl).

57. The compound of any one of claims 36-39, wherein R² is —(C₁-C₆ alkylene)-CN.

58. The compound of any one of claims 36-39, wherein R² is —(C₁-C₄ alkylene optionally substituted with OR⁵)—C(O)OR⁵.

59. The compound of any one of claims 36-39, wherein R² is —(C₁-C₄ alkylene optionally substituted with OR⁵)—C(O)N(R⁶)₂.

60. The compound of any one of claims 36-39, wherein R² is —(C₁-C₆ alkylene)-N(R⁶)₂.

61. The compound of any one of claims 36, 37, and 40-60, wherein R³ is —C₁-C₆ haloalkyl.

62. The compound of any one of claims 36, 37, and 40-61, wherein R³ is trifluoromethyl.

63. The compound of any one of claims 36, 37, and 40-60, wherein R³ is halogen.

64. The compound of any one of claims 36, 37, 40-60, and 63, wherein R³ is —F.

65. The compound of any one of claims 36, 37, and 40-64, wherein R⁴ is hydrogen.

66. The compound of any one of claims 36, 37 and 40-64, wherein R⁴ is halogen.

67. The compound of any one of claims 36, 37, 40-64, and 66, wherein R⁴ is —F.

68. The compound of any one of claims 36, 37 and 40-60, wherein R³ and R⁴ are taken together to form a 3-7 membered carbocyclic ring with the carbon to which R³ and R⁴ are attached, wherein the carbocyclic ring is optionally substituted with one or more halogens (e.g., —F).

69. The compound of claim any one of claims 1-68, wherein the compound is selected from Table 1.

70. A pharmaceutical composition comprising a compound or a pharmaceutically acceptable salt thereof of any one of claims 1-69; and a pharmaceutically acceptable excipient.

71. A method of treating a disease or condition mediated by hyperactivation of a TEAD isoform selected from TEAD1 and TEAD 4 in a subject in need thereof comprising administering to the subject, a compound or pharmaceutically acceptable salt of any one of claims 1-69 or a pharmaceutical composition of claim 70.

72. The method of claim 71, wherein the disease or condition is a cancer characterized by hyperactivation of a TEAD isoform selected from TEAD1 and TEAD4.

73. The method of claim 72, wherein the cancer is selected from the group consisting of breast cancer, lung cancer, gastric cancer, colorectal cancer, pancreatic cancer including pancreatic adenocarcinoma, mesothelioma including malignant mesothelioma, hepatocellular cancer, prostate cancer, head and neck cancer, renal cell carcinoma, and medulloblastoma.

74. The method of claim 72 or 73, wherein the cancer is selected from the group consisting of hepatocellular cancer, breast cancer, pancreatic adenocarcinoma, and malignant mesothelioma.

75. The method of any one of claims 72-74, wherein the cancer is malignant mesothelioma.

76. The method of any one of claims 72-75, wherein the cancer is metastatic.
